(19) United States
(12) Patent Application Publication
(10) Pub. No.: US 2020/0288710 A1
(43) Pub. Date: Sep. 17, 2020

(54) SUBSTITUTED SULPHONAMIDES FOR CONTROLLING ANIMAL PESTS

(71) Applicant: BAYER CROPSCIENCE AKTIENGESELLSCHAFT

(72) Inventors: Martin FÜßLEIN, Düsseldorf (DE); Heinz-Juergen WROBLOWSKY, Langenfeld (DE); Susanne KÜBBELER, Düsseldorf (DE); Dominik HAGER, Monheim (DE); Nina KAUSCH-BUSIES, Bergisch Gladbach (DE); Klaus-Helmut MÜLLER, Düsseldorf (DE); Daniela PORTZ, Vettweiß (DE); Kerstin ILG, Köln (DE); Olga MALSAM, Rösrath (DE); Sascha EILMUS, Leichlingen (DE); Peter LÖSEL, Leverkusen (DE); Ulrich GÖRGENS, Ratingen (DE); Stefan HERRMANN, Langenfeld (DE); Angela BECKER, Opio (FR)

(21) Appl. No.: 16/084,159

(22) PCT Filed: Mar. 8, 2017
(86) PCT No.: PCT/EP2017/055405

§ 371 Date: Sep. 11, 2018

(30) Foreign Application Priority Data
Mar. 15, 2016 (EP) 16160293.3

(51) Int. Cl. A01N 43/50; C07D 233/90; C07D 249/10; A01N 43/653; C07D 231/14; A01N 43/56; C07D 403/12; C07D 409/12; C07D 401/12; C07D 401/04; C07D 413/12; A01N 43/80

(52) U.S. Cl. CPC A01N 43/50; C07D 233/90; C07D 249/10; A01N 43/653; C07D 231/14; A01N 43/80; C07D 403/12; C07D 409/12; C07D 401/12; C07D 401/04; C07D 413/12; A01N 43/56

(57) ABSTRACT

The present invention relates to the use of a compound of the general formula (I)

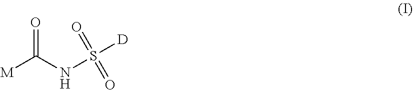

in which M and D have the meanings given in the description for controlling animal pests.

The present application relates to the use of substituted sulphonamides for controlling animal pests, to a composition comprising substituted sulphonamides for controlling animal pests, to a method for controlling animal pests, to an agrochemical formulation comprising the substituted sulphonamides, to novel substituted sulphonamides and to a process and intermediates for preparing the substituted sulphonamides.

In the literature, sulphonamides and their suitability as active compounds are described, for example, in the patent applications WO 2005/099705, WO 2003/040107, WO 2014/077285 and WO 2014/023367.

In addition, it is known, for example from the documents WO 2010/129500, WO 2012/054233, WO 2013/055584, WO 2014/109933, WO 2015/007668, WO 2015/011082 and WO 2015/169776, that certain sulphonamides can be used as nematicides.

In addition, sulphonamides which can be used as insecticides are also known from EP 2092824.

Modern insecticides have to meet many demands, for example in relation to extent, persistence and spectrum of their action and possible use. Questions of toxicity, sparing of beneficial species and pollinators, environmental properties, application rates, combinability with other active compounds or formulation auxiliaries play a role, as does the question of the effort required for the synthesis of an active compound; furthermore, resistances may occur, to mention only some parameters. For all these reasons alone, the search for novel crop protection compositions cannot be considered complete, and there is a constant need for novel compounds having improved properties compared to the known compounds, at least in relation to individual aspects.

It was an object of the present invention to provide compounds for use for controlling animal pests, which compounds widen the spectrum of the pesticides in various aspects.

This object, and further objects which are not stated explicitly but can be discerned or derived from the connections discussed herein, are achieved by the use of a compound of the formula (I)

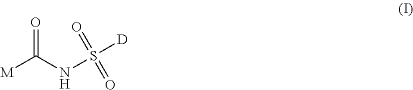

in which (configuration 0-1)
M represents a radical selected from the formulae (IIa-IIf):

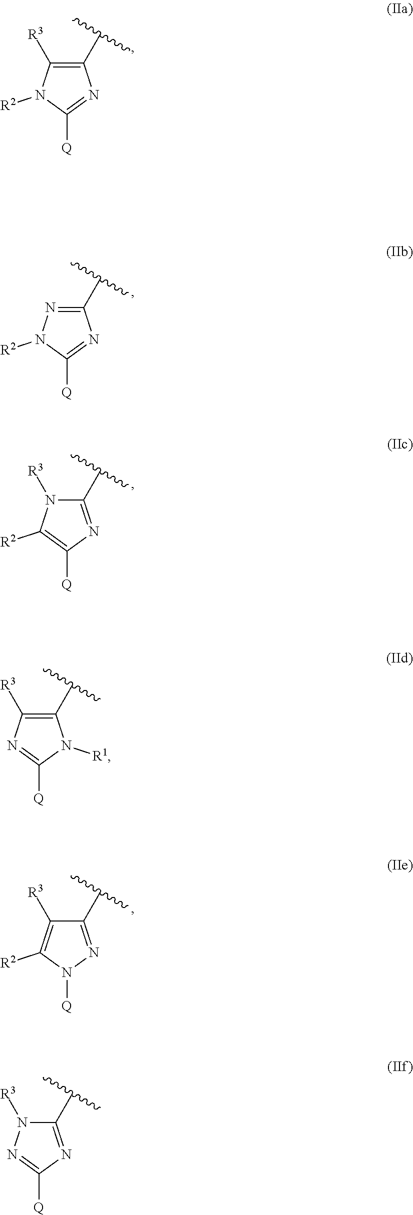

where
R¹, R², R³ each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
for controlling animal pests.

Preference is given to an embodiment of formula (I) where (range of preference 1-1)

M represents a radical selected from formulae (IIa-IIf), where R¹, R², R³ each independently of one another represent H or a substituted or unsubstituted (C₁-C₄)-alkyl, (C₃-C₆)-cycloalkyl, (C₂-C₄)alkenyl, pyridyl or phenyl radical, where in cases (IIc) and (IIe) R² may additionally represent a halogen radical or an alkoxy radical and where in cases (IIa), (IId) and (IIe) R³ may additionally represent a halogen radical;
Q represents a substituted or unsubstituted phenyl, naphthyl or heteroaryl radical which may contain one to three heteroatoms from the group consisting of oxygen, sulphur, nitrogen; but, in case (IIe), does not represent 2-pyrimidinyl; D represents a substituted or unsubstituted alkyl, heteroalkyl, cycloalkyl, heteroaryl, aryl or phenyl(C₁-C₈)-alkyl radical or a substituted or unsubstituted nitrogen radical.

More preference is given to an embodiment of formula (I) where (range of preference 2-1)

M represents a radical selected from the formulae (IIa-IIf), where R¹, R², R³ are defined as for embodiment (0-1) or range of preference (1-1) and
Q represents a phenyl, naphthyl, pyridyl, pyrimidinyl, thiophene, benzothiophene, isoquinoline, benzodioxole or pyrazole radical which is unsubstituted or substituted by one or more radicals R⁴, but, in case (IIe), does not represent 2-pyrimidinyl,
where the substituent(s) R⁴ is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino) and/or
aryl, aryloxy or hetaryl, optionally mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino and (C₁-C₆)-alkylcarbonylamino and
D represents a radical, unsubstituted or substituted by one or more radicals R⁵, from the group consisting of (C₁-C₆)-alkyl, phenyl, phenyl-(C₁-C₂)-alkyl and heteroaryl which may contain one to three heteroatoms from the group consisting of oxygen, sulphur, nitrogen, or represents an NR⁶R⁷ radical, where the substituent(s) R⁵ is/are each independently of one another selected from: cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino) and (1-pyrazolyl)-(C₁-C₃)-alkyl
and/or
aryl or hetaryl, mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)alkylsilyl, (C₃-C₈)-cycloalkyl, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino and (C₁-C₆)-alkylcarbonylamino
and where R⁶ and R⁷ each independently of one another represent H, (C₁-C₆)-alkyl or a substituted or unsubstituted phenyl radical or R⁶ and R⁷ together may form an unsubstituted or substituted 4- to 8-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R⁵.

Even more preferably (range of preference 3-1)

M represents a radical selected from the formulae (IIa-IIf), where R¹, R², R³ are defined as for embodiment (0-1) or range of preference (1-1) and
Q represents a phenyl, naphthyl, pyridyl, pyrimidinyl, thiophene, benzothiophene, isoquinoline, benzodioxole or pyrazole radical which is unsubstituted or substituted by one or more radicals R⁴, but, in case (IIe), does not represent 2-pyrimidinyl,
where the substituent(s) R⁴ is/are each independently of one another selected from:
cyano, halogen, nitro, acetyl, hydroxy, carboxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)alkylsulphonyl-(C₁-C₆)-alky, (C₁-C₆)-alkylsulphonyloxy, (C₁-C₆)-alkylcarbonyl, (C₁-C₆)alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)alkylaminothiocarbonyl, di-(C₁-C₆)-alkylaminocarbonyl, di-(C₁-C₆)-alkylaminothiocarbonyl, (C₂-C₆)alkenylaminocarbonyl, di-(C₂-C₆)-alkenylaminocarbonyl, (C₃-C₈)-cycloalkylaminocarbonyl, (C₁-C₆)alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, phenyl, halophenyl, phenoxy or halophenoxy and
D represents a radical, unsubstituted or substituted by one or more radicals R⁵, from the group consisting of (C₁-C₆)-alkyl, phenyl, phenyl-(C₁-C₂)-alkyl and heteroaryl which may contain one to three heteroatoms from the group consisting of oxygen, sulphur and nitrogen, or represents an NR⁶R⁷ radical,
where the substituent(s) R⁵ is/are each independently of one another selected from:
cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)-alkylhydroxyimino, (C₁-C₆)alkoxyimino, (C₁-C₆)-alkyl-(C₁-C₆)-alkoxyimino, (C₁-C₆)-haloalkyl-(C₁-C₆)-alkoxyimino, (C₁-C₆)alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl) (C₁-C₃)-alkyl and
R⁶ and R⁷ each independently of one another represent H, a (C₁-C₆)-alkyl, (C₁-C₆)-haloalkyl, (C₁-C₆)alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl, (C₁-C₆)-alkoxy-(C₁-C₆)-alkyl,
or
R⁶ and R⁷ together may form an unsubstituted or substituted 5- to 6-membered saturated or optionally fully or partially unsaturated ring which may be interrupted by 1 to 3 heteroatoms from the group consisting of oxygen, sulphur and nitrogen and which may be provided by one or more substitutions corresponding to one of the definitions of R⁵.

Special preference is given to an embodiment of formula (I) where (range of preference 4-1)

M represents a radical selected from the formulae (IIa-IIf), where
in case (IIa) R², R³ each independently of one another represent H or a substituted or unsubstituted (C₁-C₄)-alkyl, (C₃-C₆)-cycloalkyl or phenyl radical, where R³ may additionally represent a halogen radical,
in case (IIb) R² represents H or a substituted or unsubstituted (C₁-C₄)-alkyl or (C₃-C₆)-cycloalkyl radical,
in case (IIc) R², R³ each independently of one another represent H or a substituted or unsubstituted (C₁-C₄)-alkyl, (C₃-C₆)-cycloalkyl or phenyl radical, and R² may additionally represent a halogen or (C₁-C₄)-alkoxy radical,
in case (IId) R¹, R³ each independently of one another represent H or a substituted or unsubstituted (C₁-C₄)-alkyl or (C₃-C₆)-cycloalkyl radical, where R³ may additionally represent a halogen radical,
in case (IIe) R², R³ each independently of one another represent H, halogen or a substituted or unsubstituted (C₁-C₄)-alkyl, (C₃-C₆)-cycloalkyl, pyridyl or phenyl radical, and R² may additionally represent a (C₁-C₄)-alkoxy radical and
in case (IIf) R³ represents H or a substituted or unsubstituted (C₁-C₄)-alkyl or (C₃-C₆)-cycloalkyl radical
and Q is defined as in embodiment (0-1), range of preference (1-1), range of preference (2-1) or range of preference (3-1) and
D represents a (C₁-C₆)-alkyl, phenyl, naphth-2-yl, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R⁵ or represents an NR⁶R⁷ radical,
where the substituent(s) R⁵ is/are each independently of one another selected from:
cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)-alkylhydroxyimino, (C₁-C₆)alkoxyimino, (C₁-C₆)-alkyl-(C₁-C₆)-alkoxyimino, (C₁-C₆)-haloalkyl-(C₁-C₆)-alkoxyimino, (C₁-C₆)alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, (1-pyrazolyl)-(C₁-C₃)-alkyl and
R⁶ and R⁷ each independently of one another represent H, a (C₁-C₆)-alkyl, (C₁-C₆)-haloalkyl, (C₁-C₆)alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl or (C₁-C₆)-alkoxy-(C₁-C₆)-alkyl,
or form a ring from the group pyrrolidine, morpholine, piperidine.

Even more preference is given to an embodiment of formula (I) where (range of preference 5-1)

M represents a radical selected from the radicals of the formulae (IIa) to (IIf) where
in case (IIa) R² represents H, methyl or ethyl or optionally halogen-substituted phenyl and R³ represents H, methyl, ethyl, isopropyl or halogen,
in case (IIb) R² represents H, methyl or ethyl,
in case (IIc) R² represents H or methyl and R³ represents H, methyl or ethyl,
in case (IId) R¹ represents H or methyl and R³ represents H or halogen,
in case (IIe) R² represents H, methyl, methoxy, halogen-substituted phenyl or halogen-substituted pyridyl and R³ represents H,
in case (IIf) R³ represents H, methyl or ethyl
and Q is defined as in embodiment (0-1), range of preference (1-1), range of preference (2-1) or range of preference (3-1).

Very particular preference is given to an embodiment of formula (I) where (range of preference 6-1)

M is selected from one of the formulae (IIa) to (IIf) or (IVa) to (IVf), where R¹ to R³ are as defined in embodiment (0-1), range of preference (1-1), range of preference (4-1) or range of preference (5-1) and
Q represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R⁴, but, in case (IVe), does not represent 2-pyrimidinyl,
where the substituent(s) R⁴ independently of one another represent(s):
cyano, halogen, nitro, acetyl, (C₃-C₆)-cycloalkyl, (C₃-C₆)-cycloalkyloxy, (C₃-C₆)-cycloalkyl-(C₃-C₆)cycloalkyl, (C₁-C₆)-alkyl-(C₃-C₈)-cycloalkyl, halo-(C₃-C₆)-cycloalkyl, (C₁-C₆)-alkyl, (C₁-C₆)haloalkyl, (C₁-C₆)-cyanoalkyl, (C₁-C₆)-hydroxyalkyl, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)haloalkoxy, (C₁-C₆)-alkylthio, (C₁-C₆)-haloalkylthio, (C₁-C₆)-alkylthio-(C₁-C₆)-alkyl, (C₁-C₆)alkylsulphinyl, (C₁-C₆)-haloalkylsulphinyl, (C₁-C₆)-alkylsulphonyl, (C₁-C₆)-haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)alkylcarbonylamino, phenyl, halophenyl, phenoxy or halophenoxy and
D represents a (C₁-C₆)-alkyl, phenyl, naphth-2-yl, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R⁵ or represents an NR⁶R⁷ radical,
where the substituent(s) R⁵ independently of one another represent(s):
cyano, halogen, nitro, acetyl, hydroxy, carboxy, amino, (C₃-C₆)-cycloalkyl, (C₃-C₆)-cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)-hydroxyalkyl, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)alkyl, (C₁-C₆)-alkylsulphinyl, (C₁-C₆)-haloalkylsulphinyl, (C₁-C₆)-alkylsulphonyl, (C₁-C₆)haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino, (1-pyrazolyl)-(C₁-C₃)-alkyl,
R⁶ and R⁷ each independently of one another represent H, (C₁-C₆)-alkyl, phenyl, alkoxyphenyl or halophenyl or form a ring from the group
pyrrolidine, morpholine, piperidine.

Even more preference is given to a configuration of formula (I) where (range of preference 7-1)

M represents a radical selected from the radicals of the formulae (Va-Vz):

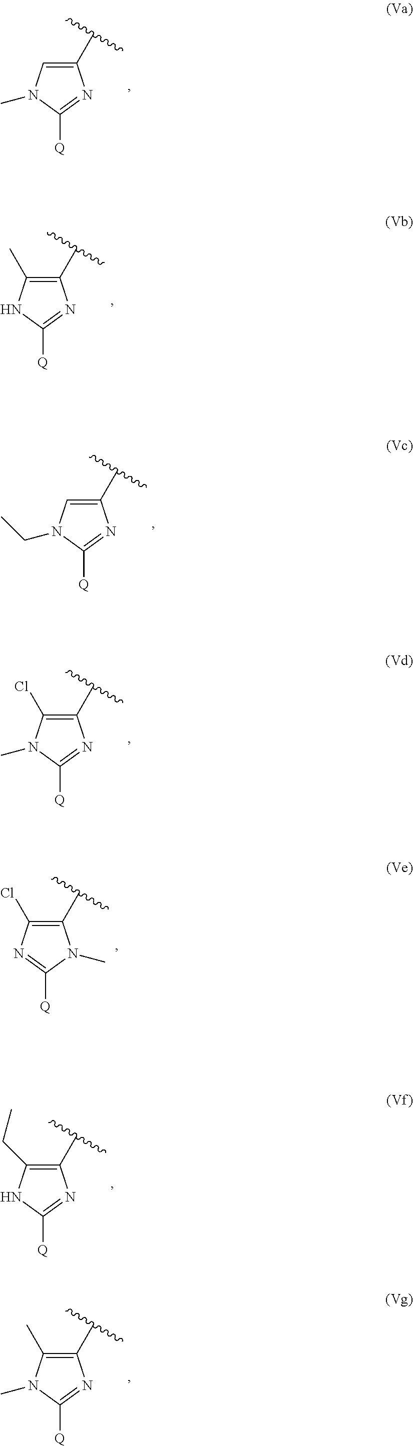

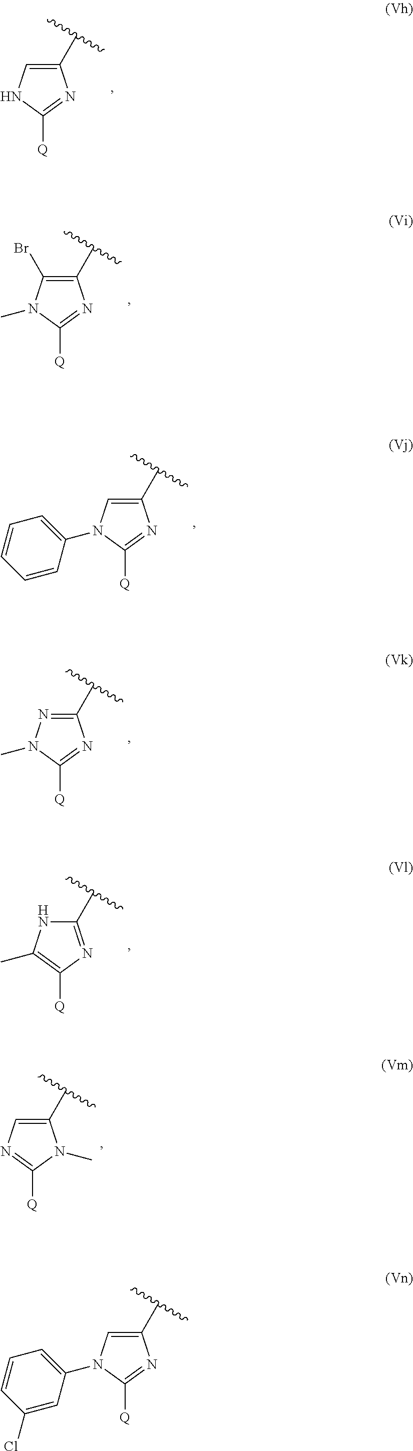

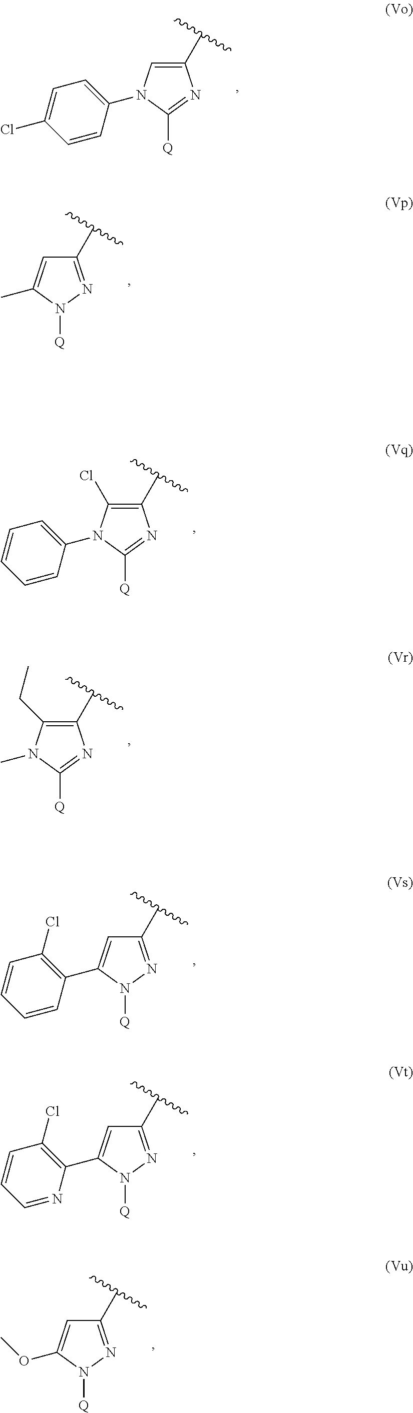

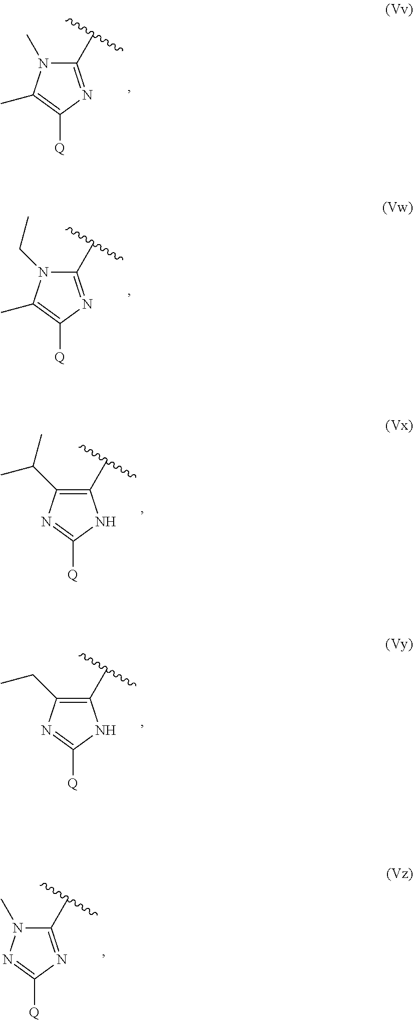

Q represents a radical selected from the radicals of the formulae (VIa-VIz and VIa1-VIa30):

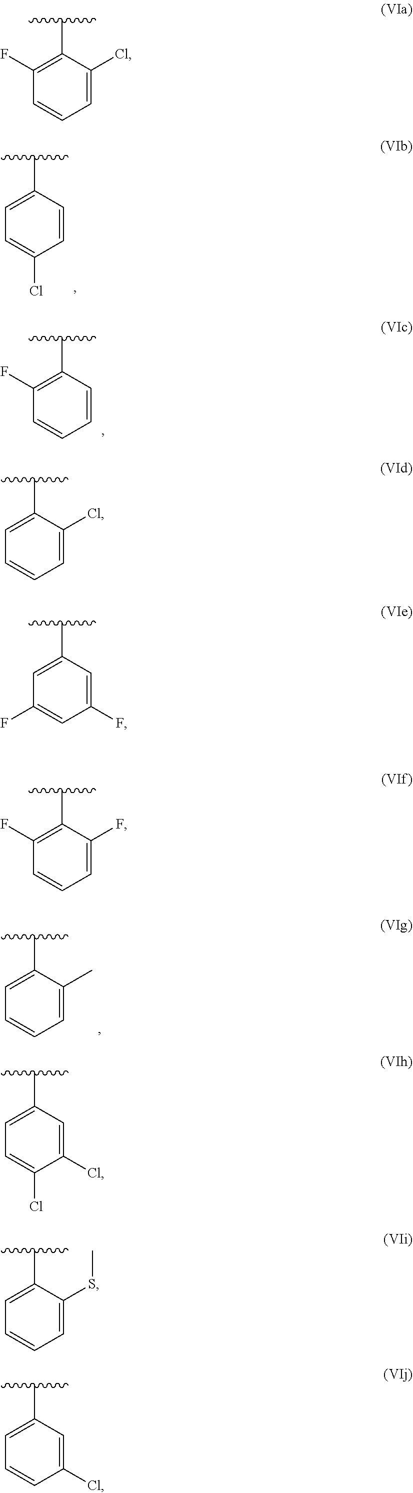

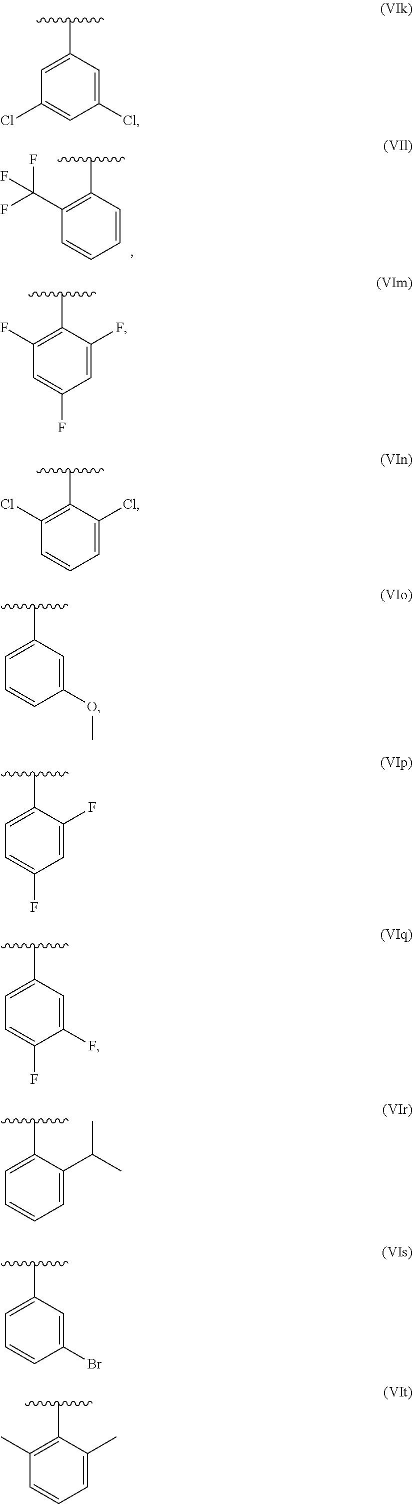

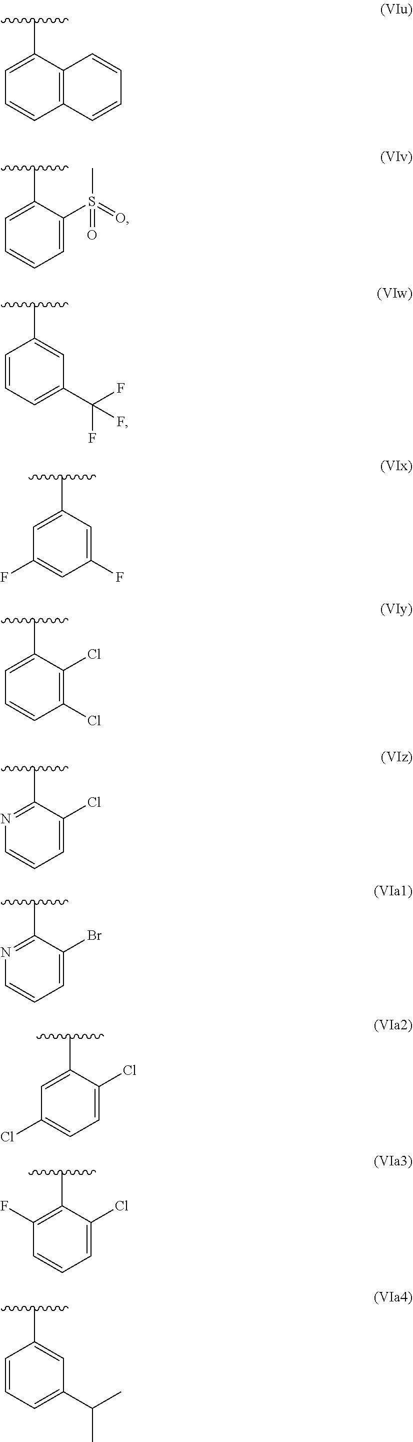

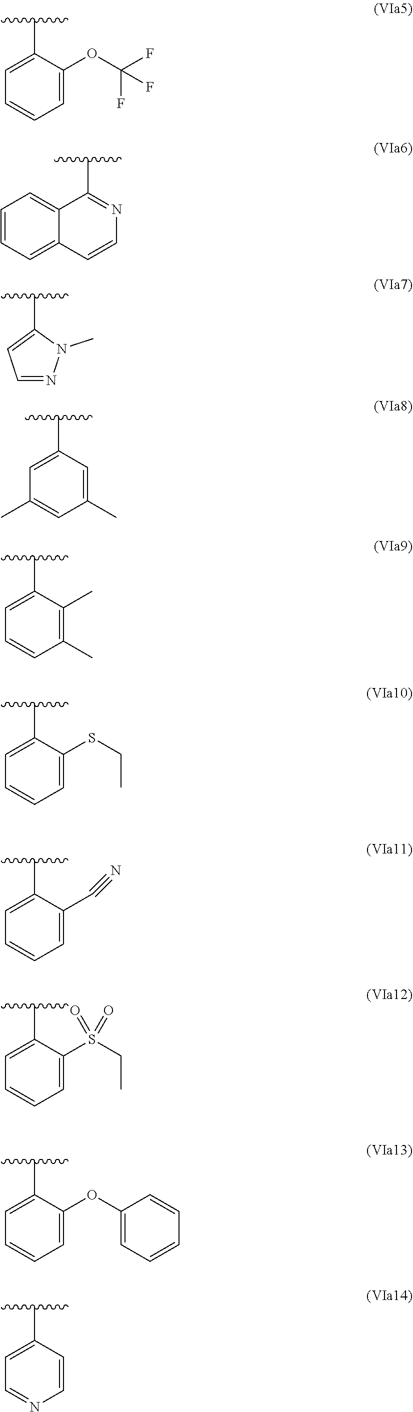

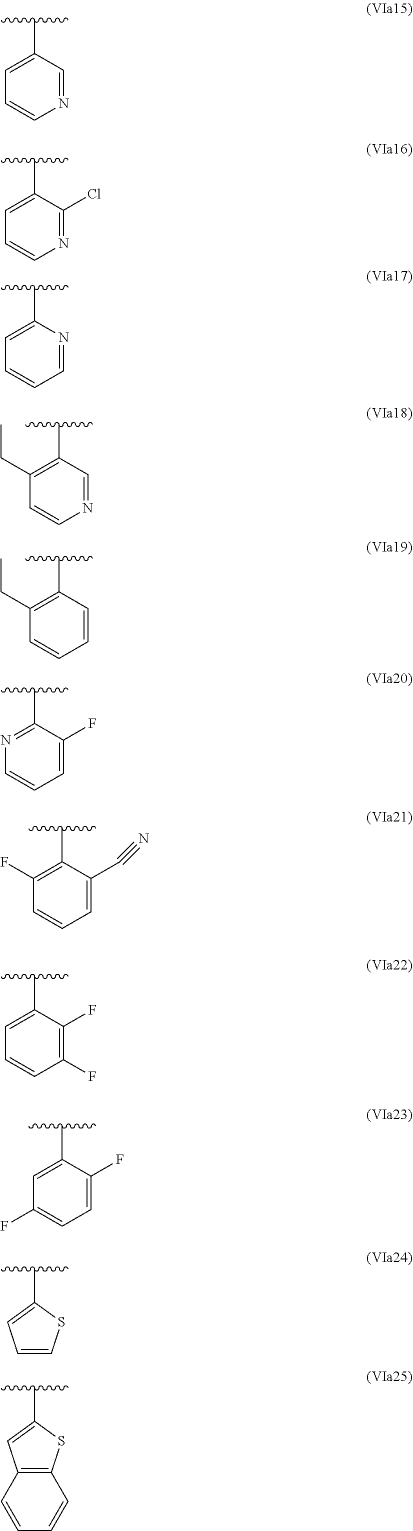

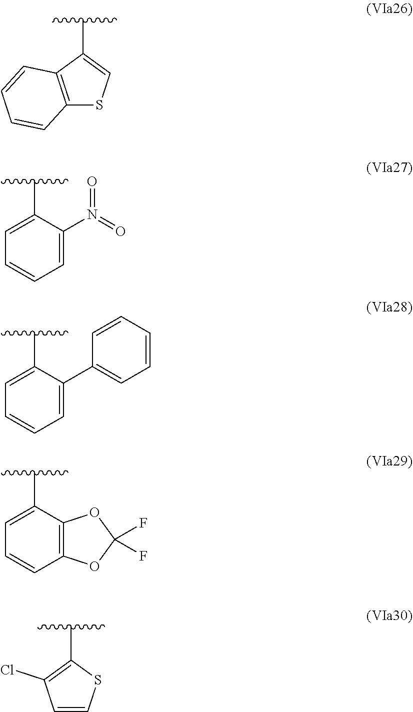

and
D represents a radical selected from the radicals of the formulae (VII1-VII192)

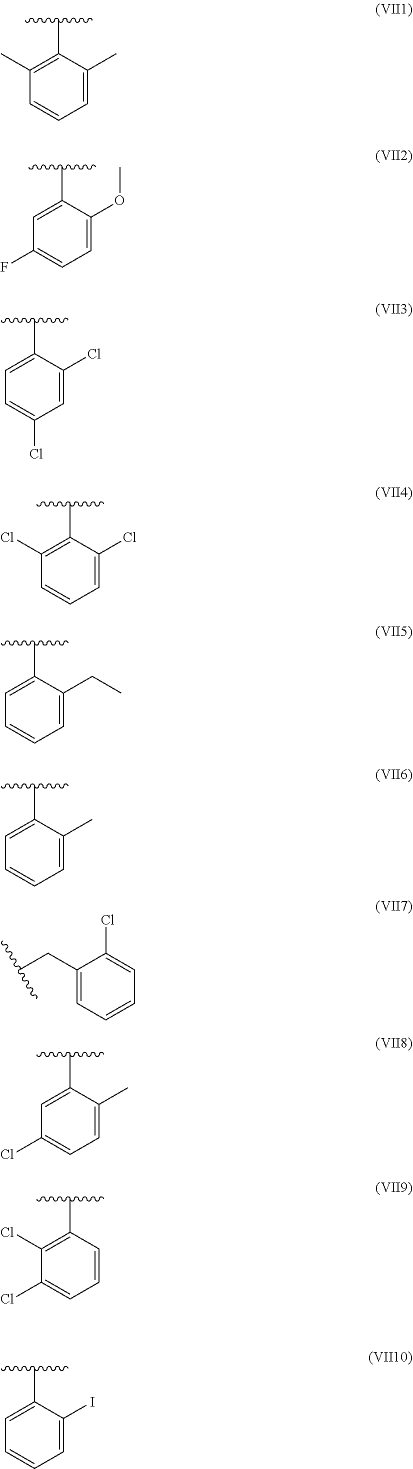

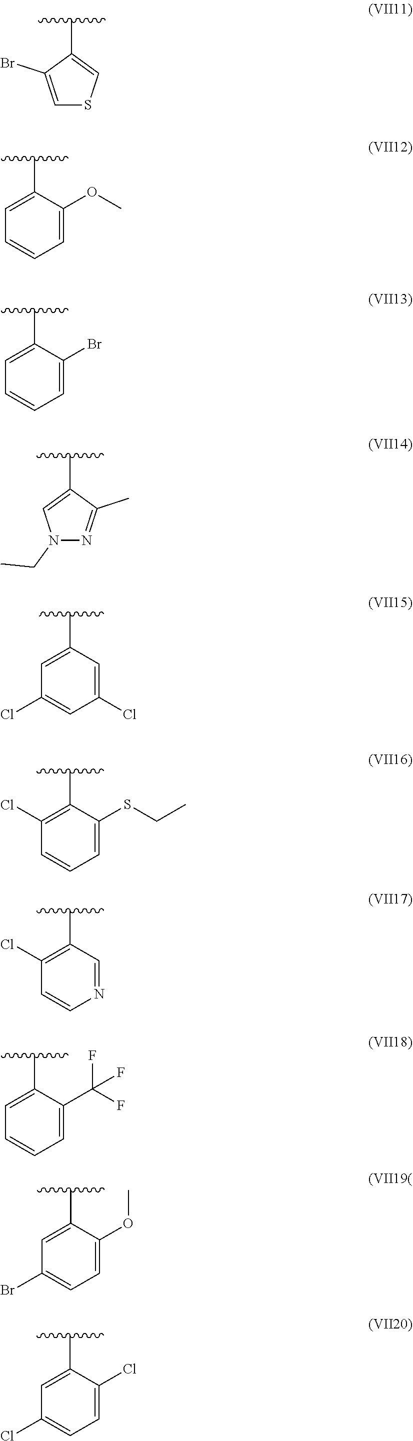

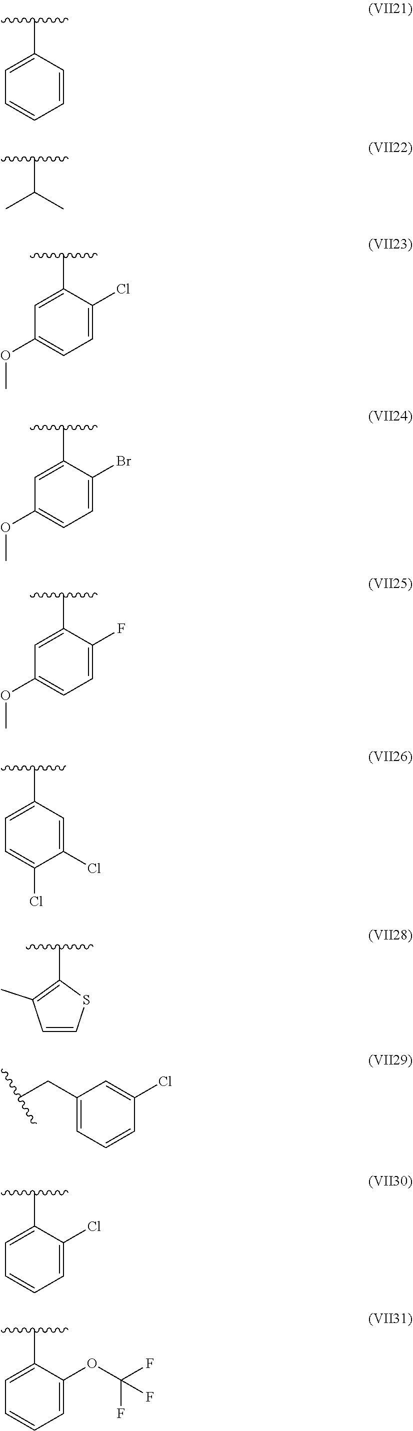

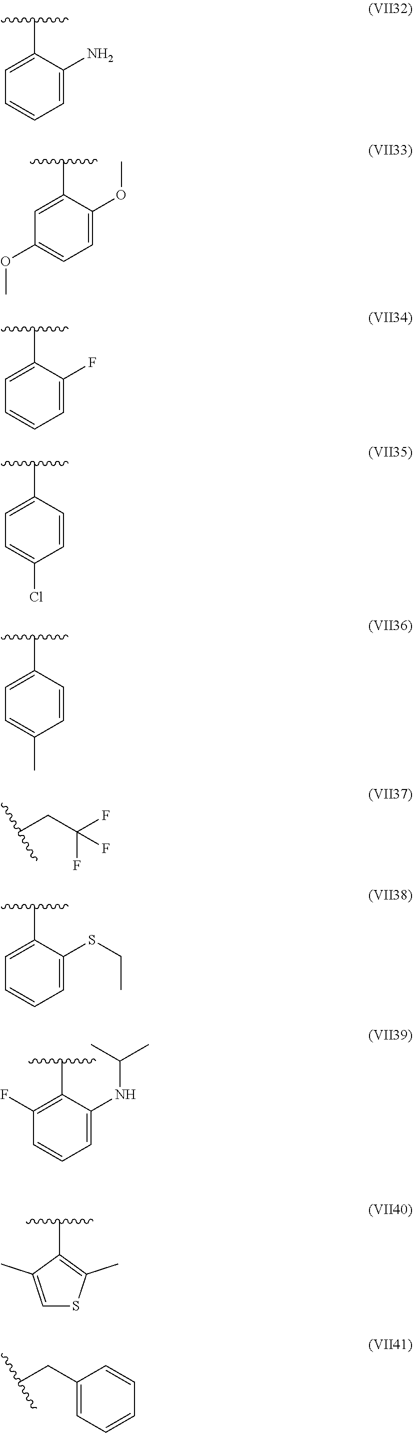

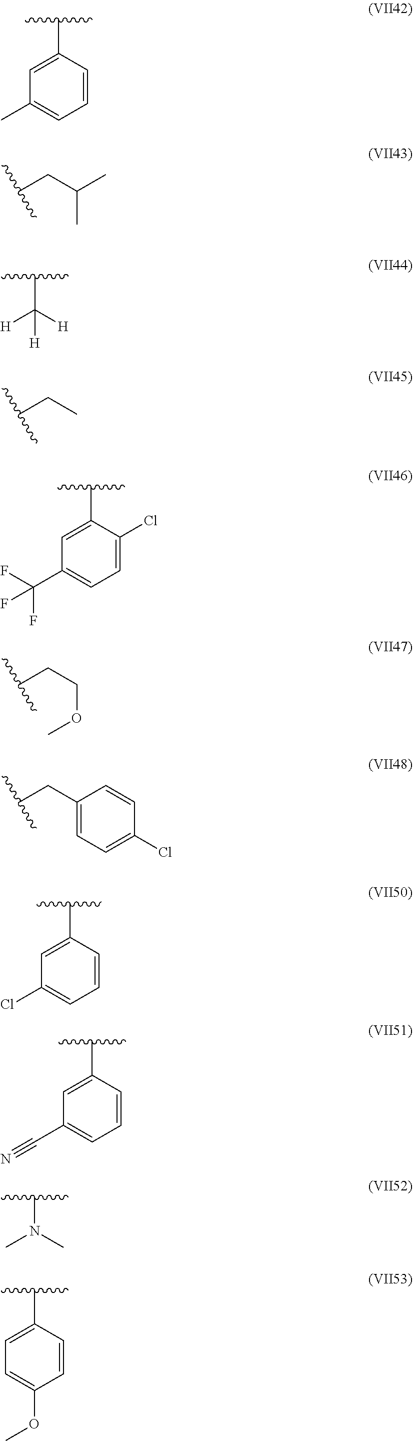

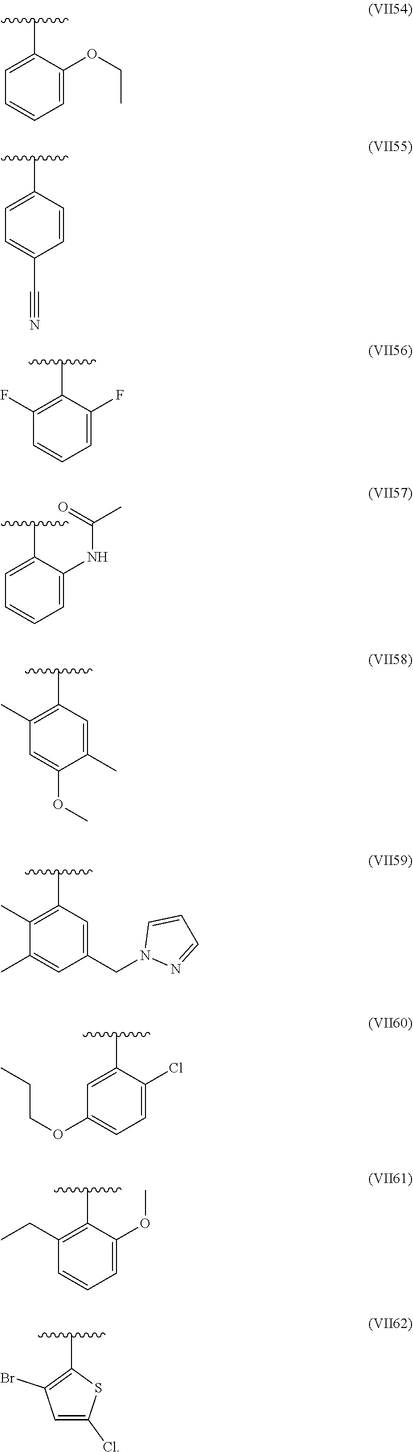

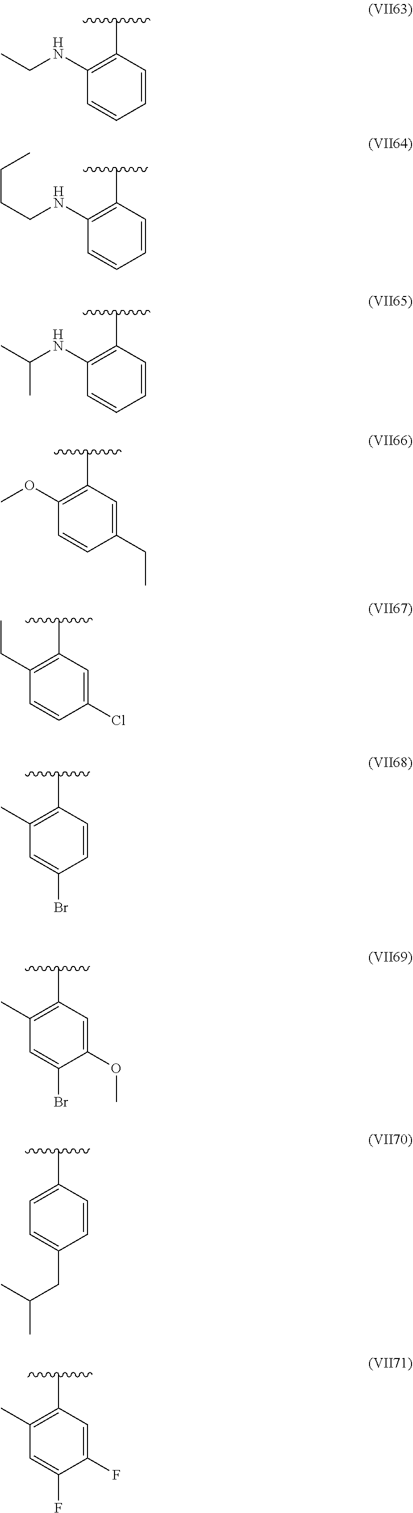

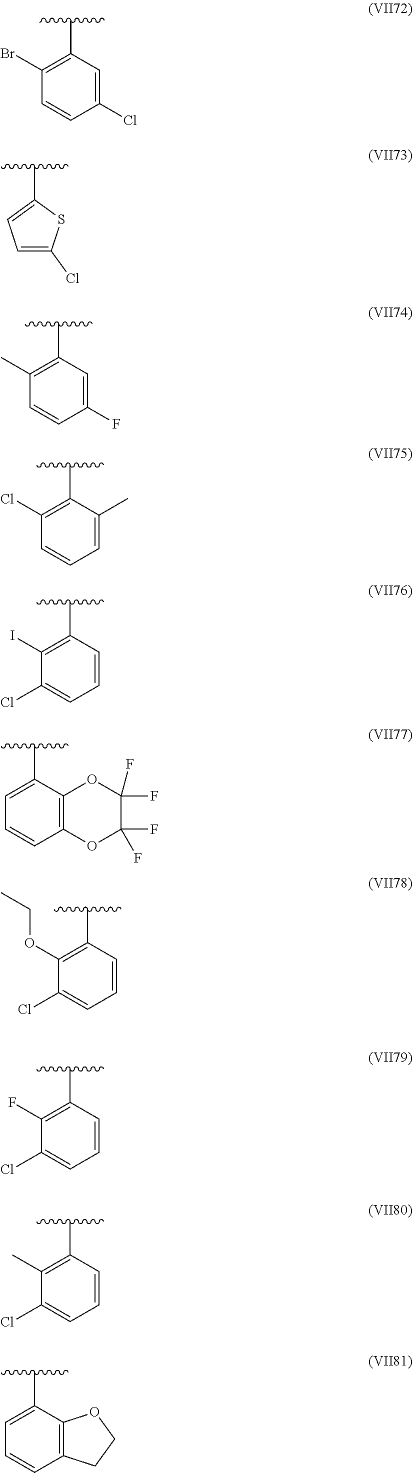

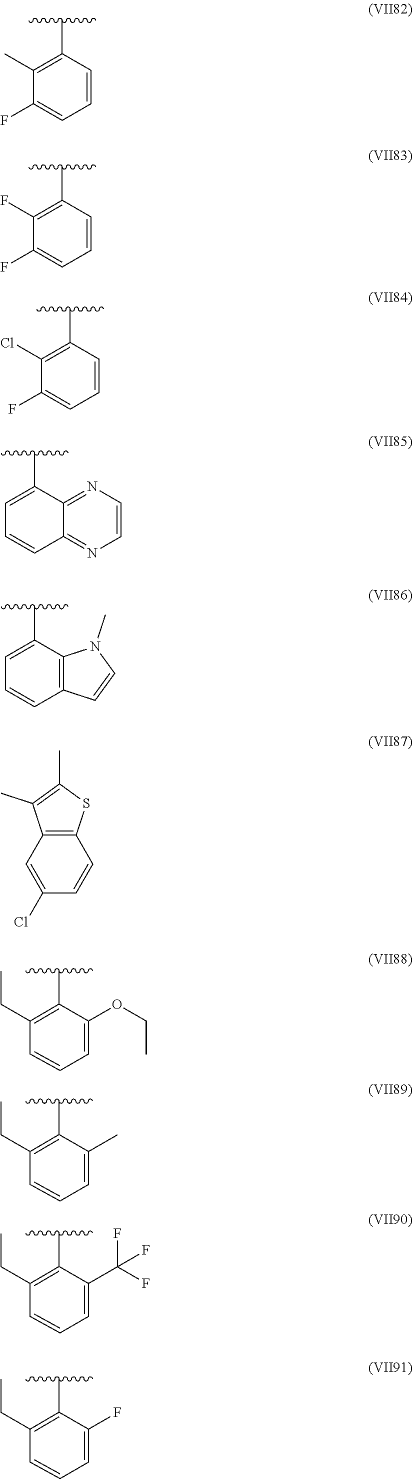

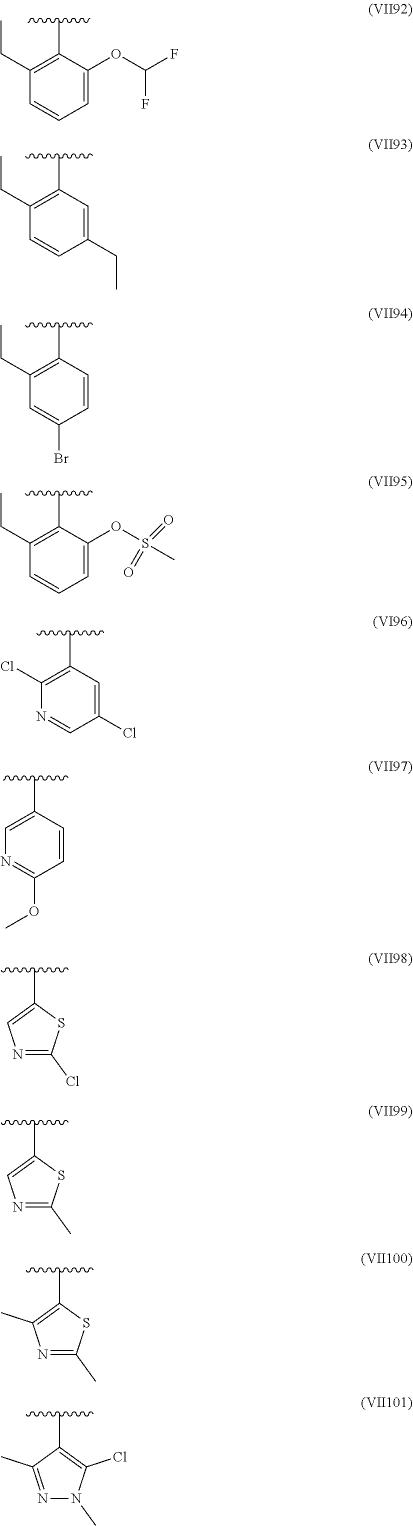

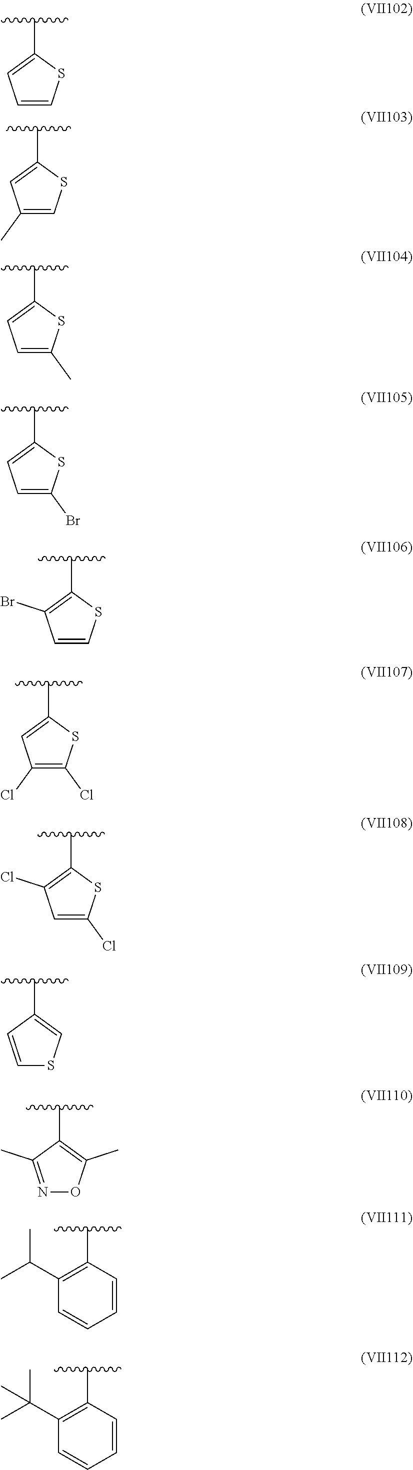

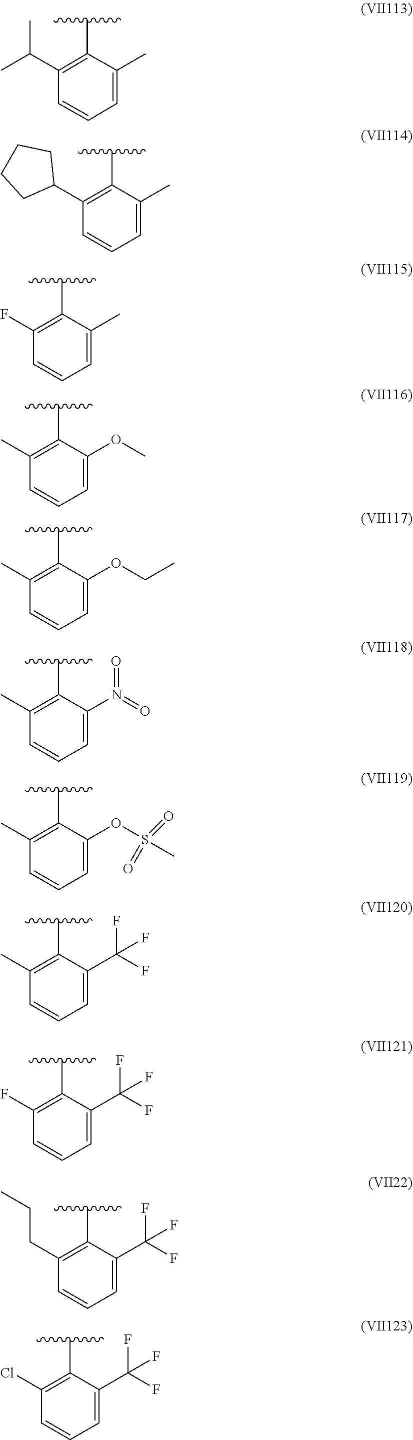

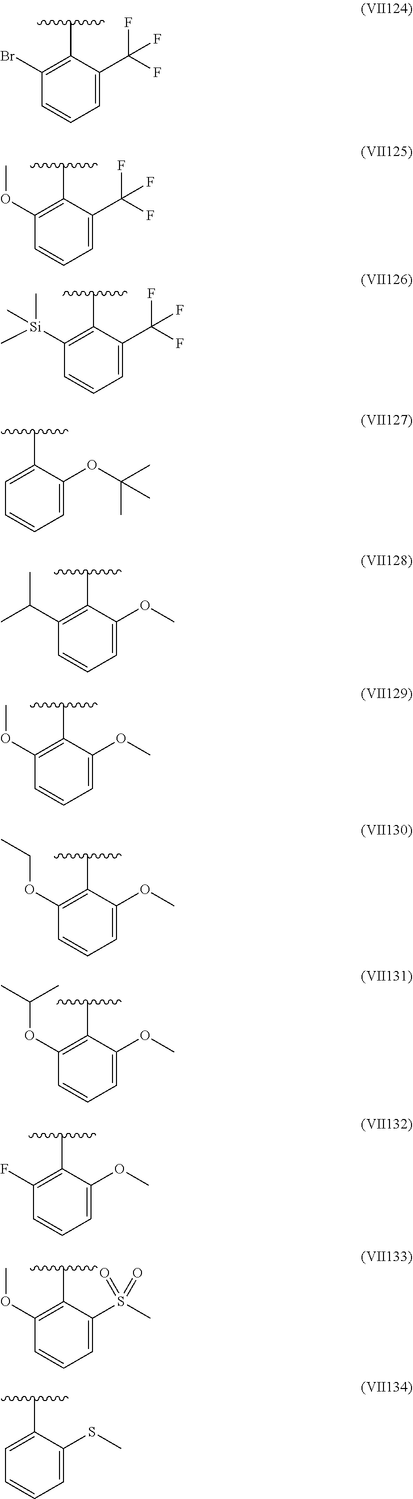

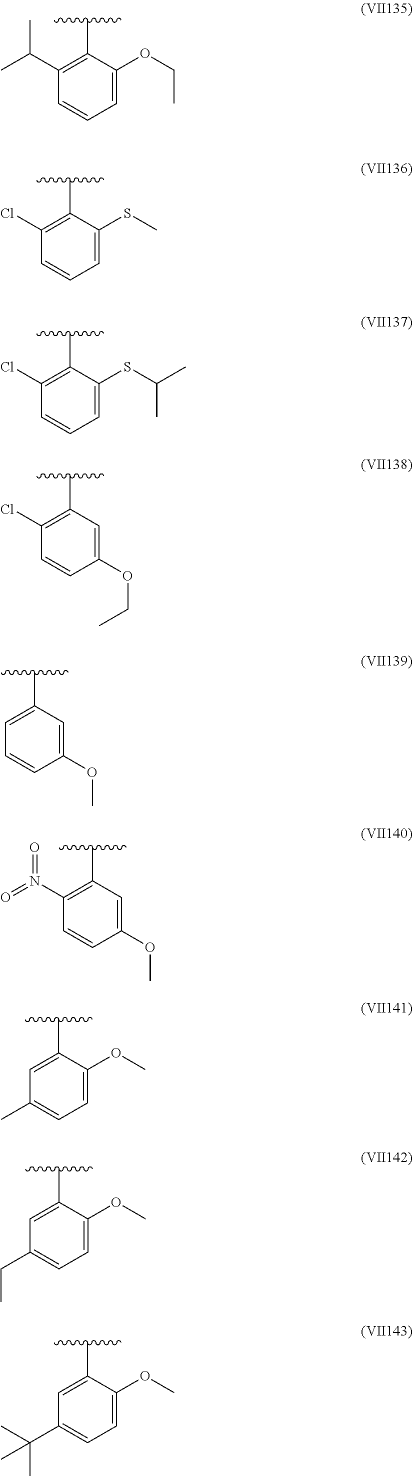

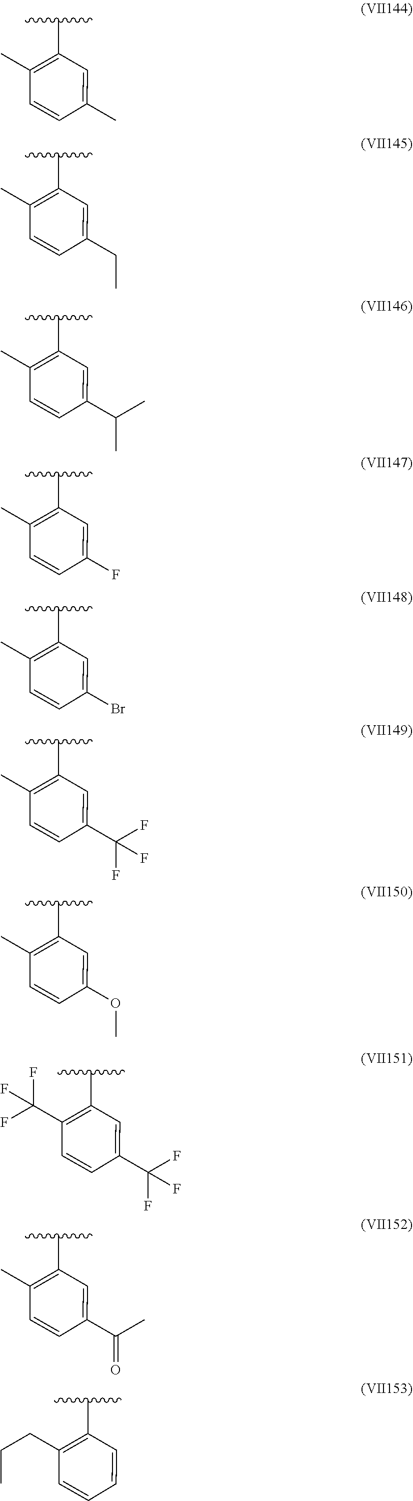

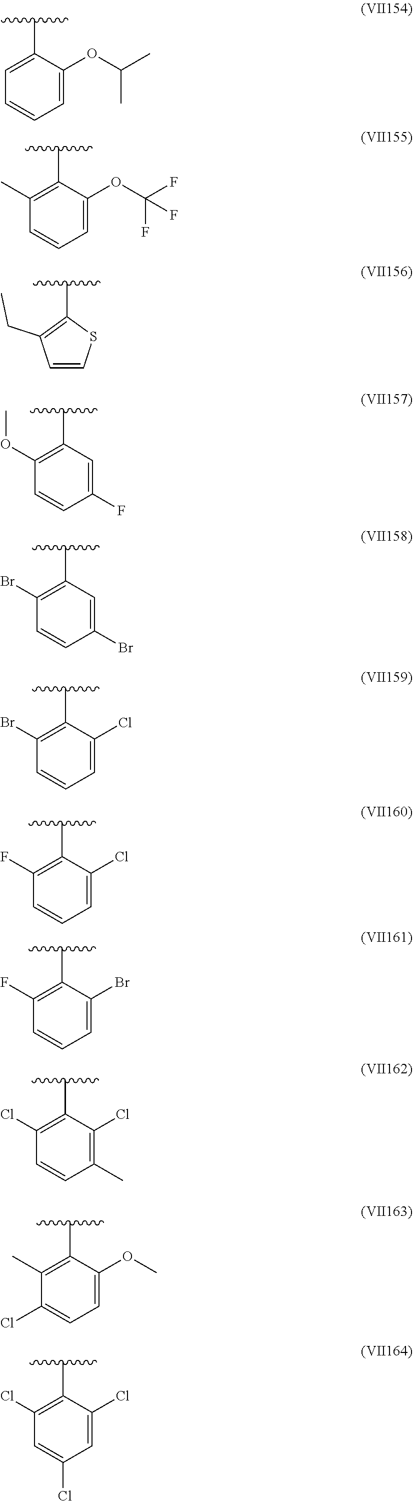

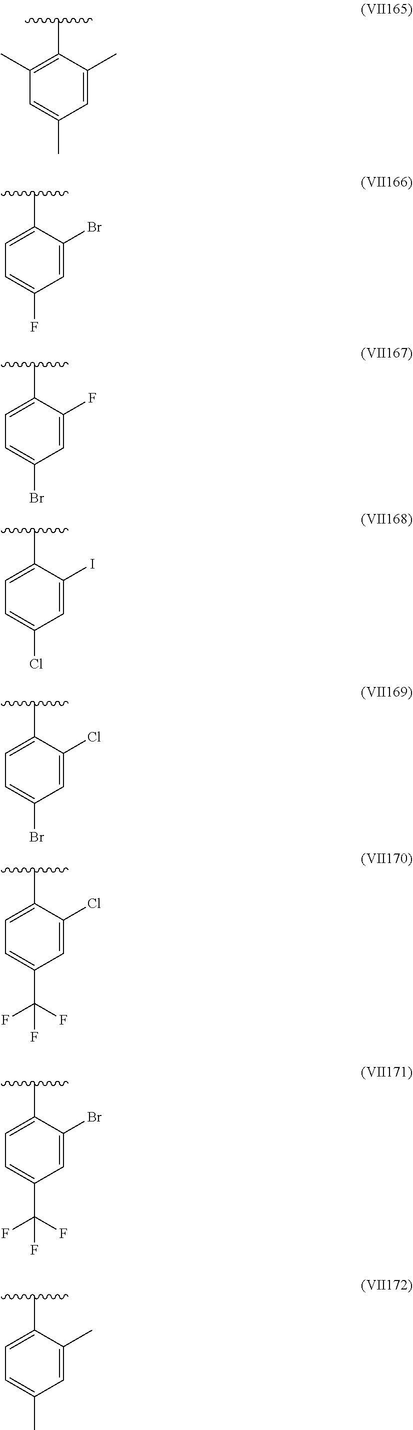

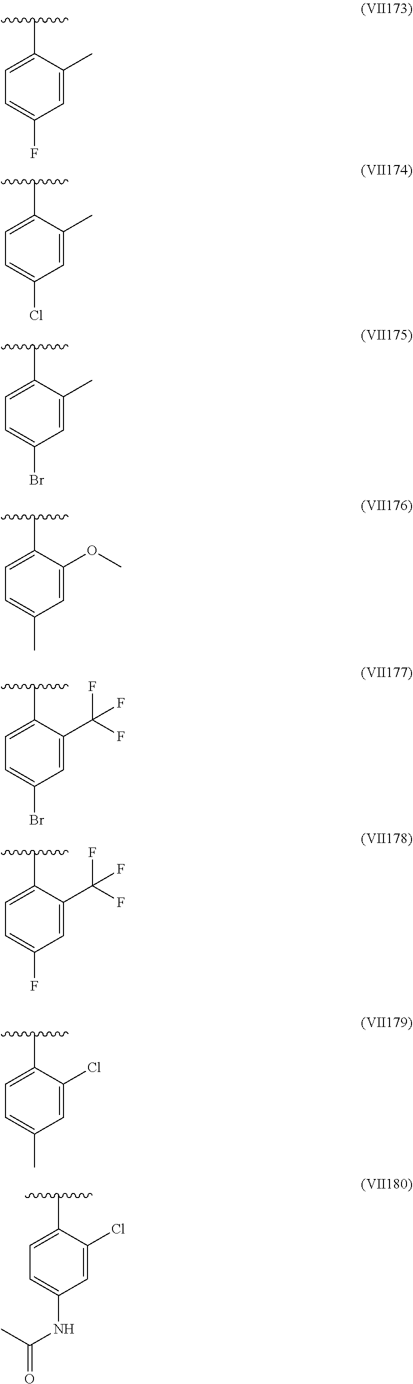

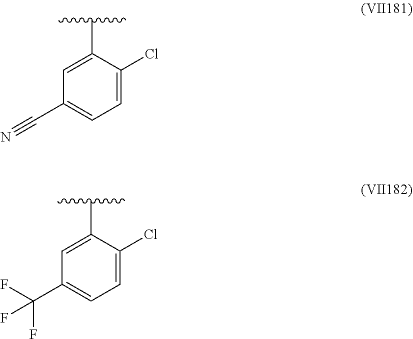 [SMILES: *c1cc(C#N)ccc1Cl ; *c1cc(C(F)(F)F)ccc1Cl]

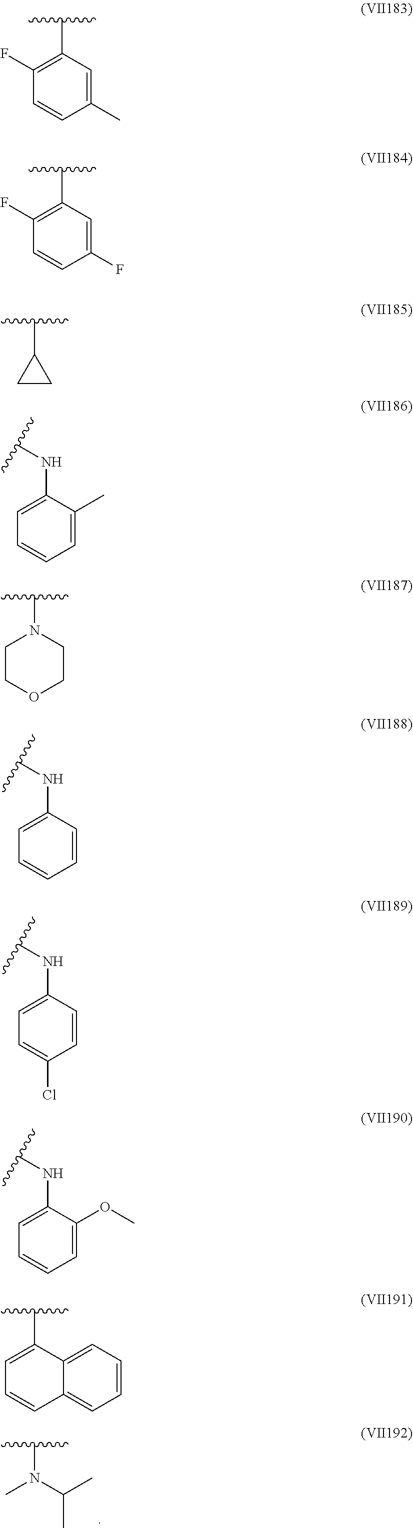

According to a further preferred embodiment of the invention, the use of a compound of the formula (I) for protecting the propagation material of plants is provided.

The invention also provides a composition comprising at least one compound of the formula (I) and customary extenders and/or surfactants, in particular for controlling animal pests.

The invention furthermore provides a method for controlling animal pests, in which at least one compound of the formula (I) or a composition according to the invention is allowed to act on the animal pests and/or their habitat.

According to a preferred embodiment of the method, the surgical, therapeutic and diagnostic treatment of the human or animal body is excluded.

The invention still furthermore provides an agrochemical formulation comprising at least one compound of the formula (I) according to the invention in biologically effective amounts of from 0.00000001 to 98% by weight, based on the weight of the agrochemical formulation, and also extenders and/or surfactants.

A preferred embodiment of the formulation according to the invention additionally comprises a further agrochemically active compound.

The invention likewise provides compounds of the formula (VIII)

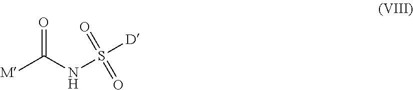

in which
M′ represents a radical of the formula (II) selected from:

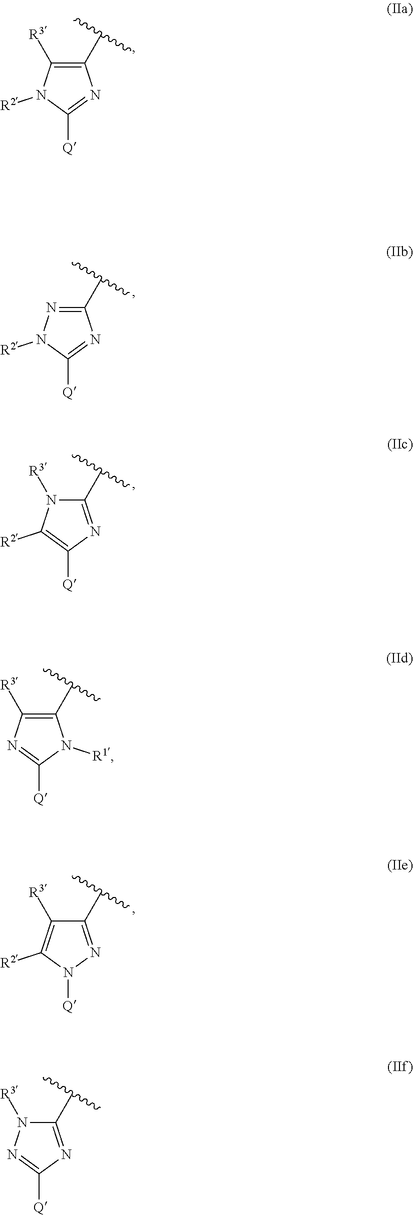

in which (configuration 0-2)
R^{1′}, R^{2′}, R^{3′} each independently of one another represent H or a substituted or unsubstituted alkyl, cycloalkyl, alkenyl, cycloalkenyl, cycloheteroalkyl, aryl or heteroaryl radical, where in cases (IIc) and (IIe) R^{2′} may additionally represent a halogen radical or an alkoxy radical, in cases (IIa) and (IIb) R^{2′} may only represent H or a substituted or unsubstituted alkyl or cycloalkyl radical and in cases (IIa), (IId) and (IIe) R^{3′} may additionally represent a halogen radical,
Q′ represents a substituted or unsubstituted aryl or heteroaryl radical, but in cases (IIa), (IId) in case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl, nor 3,5-bis-tert-butyl;
D′ represents a substituted or unsubstituted alkyl, heteroalkyl, optionally partially unsaturated cycloalkyl, cycloheteroalkyl, heteroaryl, aryl or phenylalkyl radical or in the case that Q′ carries at least one substituent in the 2-position represents a substituted or unsubstituted nitrogen radical.

According to a first preferred embodiment (range of preference 1-2) of the compounds according to the invention,

M represents a radical of one of the formulae (IIa) to (IIf) and
in case (IIa) R^{2′}, R^{3′} each independently of one another represent H or a substituted or unsubstituted (C₁-C₄)-alkyl, (C₃-C₆)-cycloalkyl or phenyl radical, where R^{3′} may additionally represent a halogen radical;
in case (IIb) R^{2′} represents H or a substituted or unsubstituted (C₁-C₄)-alkyl or (C₃-C₆)-cycloalkyl radical,
in case (IIc) R^{2′}, R^{3′} each independently of one another represent H or a substituted or unsubstituted (C₁-C₄)-alkyl, (C₃-C₆)-cycloalkyl or phenyl radical, where R^{2′} may additionally represent a halogen radical or a (C₁-C₄)-alkoxy radical,
in case (IId) R^{2′}, R^{3′} each independently of one another represent H or a substituted or unsubstituted (C₁-C₄)-alkyl or (C₃-C₆)-cycloalkyl radical und R^{3′} may additionally represent a halogen radical,
in case (IIe) R^{2′}, R^{3′} each independently represent H, halogen or a substituted or unsubstituted (C₁-C₄)alkyl, (C₃-C₆)-cycloalkyl, pyridyl or phenyl radical, and R^{2′} may additionally represent a (C₁-C₄)alkoxy radical and
in case (IIf) R^{3′} represents H or a substituted or unsubstituted (C₁-C₄)-alkyl or (C₃-C₆)-cycloalkyl radical,
Q′ represents a substituted or unsubstituted phenyl, naphthyl or heteroaryl radical which may contain one to three heteroatoms from the group of oxygen, sulphur and nitrogen, but in cases (IIa), (IId) in case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl,
D′ represents a substituted or unsubstituted alkyl, heteroalkyl, cycloalkyl, heteroaryl, aryl or phenyl(C₁-C₈)-alkyl radical or, in the case that Q′ carries at least one substituent in the 2-position, represents a substituted or unsubstituted nitrogen radical.

More preferably, for the compounds of the formula (VIII) according to the invention (range of preference 2-2)

M′ represents a radical selected from the formulae (IIa-IIf), where R^{1′}, R^{2′}, R^{3′} are defined as for embodiment (0-2) or range of preference (1-2) and
Q′ represents a phenyl, naphthyl, pyridyl, pyrimidinyl, thiophene, benzothiophene, isoquinoline, benzodioxole or pyrazole radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituent(s) R^{4′} is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and/or
aryl, aryloxy or hetaryl, optionally mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino and (C₁-C₆)-alkylcarbonylamino and
D′ represents a radical, unsubstituted or substituted by one or more radicals R^{5′}, from the group consisting of (C₁-C₆)-alkyl, phenyl and heteroaryl which may contain one to three heteroatoms from the group consisting of oxygen, sulphur and nitrogen, or, in the case that Q′ carries at least one substituent in the 2-position, represents an NR^{6′}R^{7′} radical,
where the substituent(s) R^{5′} is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl
and/or
aryl or hetaryl, mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)alkylsilyl, (C₃-C₈)-cycloalkyl, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino and (C₁-C₆)-alkylcarbonylamino,
and where R^{6′} and R^{7′} each independently of one another represent H, a (C₁-C₆)-alkyl, (C₁-C₆)haloalkyl, (C₁-C₆)-alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl, (C₁-C₆)-alkoxy-(C₁-C₆)alkyl, or
R^{6′} and R^{7′} together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R^{5′}.

Even more preferably (range of preference 3-2)

M′ represents a radical selected from the formulae (IIa-IIf), where R¹, R^{2′}, R^{3′} are defined as for embodiment (0-2) or range of preference (1-2) and
Q′ represents a phenyl, naphthyl, pyridyl, pyrimidinyl, thiophene, benzothiophene, isoquinoline, benzodioxole or pyrazole radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl,
and the substituent(s) R^{4′} are each independently of one another selected from: cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)alkylsulphonyl-(C₁-C₆)-alky, (C₁-C₆)-alkylsulphonyloxy, (C₁-C₆)-alkylcarbonyl, (C₁-C₆)alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)alkylaminothiocarbonyl, di-(C₁-C₆)-alkylaminocarbonyl, di-(C₁-C₆)-alkylaminothiocarbonyl, (C₂-C₆)alkenylaminocarbonyl, di-(C₂-C₆)-alkenylaminocarbonyl, (C₃-C₈)-cycloalkylaminocarbonyl, (C₁-C₆)alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, phenyl, halophenyl, phenoxy or halophenoxy and
D′ represents a phenyl, pyridine, pyrimidine, pyrazole, triazole, thiazole, oxazole, thiadiazole, oxadiazole, thiophene, pyrrole, furan, tetrahydrofuran, dioxane or (C₁-C₆)-alkyl radical which is unsubstituted or substituted by one or more radicals R^{5′}, or represents an NR^{6′}R⁷ radical,
where the substituent(s) R^{5′} is/are each independently of one another selected from:
cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)-alkylhydroxyimino, (C₁-C₆)alkoxyimino, (C₁-C₆)-alkyl-(C₁-C₆)-alkoxyimino, (C₁-C₆)-haloalkyl-(C₁-C₆)-alkoxyimino, (C₁-C₆)alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl,
and where R^{6′} and R^{7′} each independently of one another represent H, a (C₁-C₄)-alkyl, (C₁-C₄)haloalkyl, (C₁-C₄)-alkoxy-(C₁-C₄)-alkyl radical or an unsubstituted phenyl radical or a phenyl radical substituted by halogen, (C₁-C₄)-alkyl, (C₁-C₄)-alkoxy, (C₁-C₄)-haloalkyl, (C₁-C₄)-alkoxy-(C₁-C₄)alkyl, or
R^{6′} and R^{7′} together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R^{5′}.

Special preference is given to an embodiment of formula (VIII) where (range of preference 4-2)

D′ represents a (C₁-C₆)-alkyl radical, phenyl radical, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R^{5′} or represents an NR^{6′}R^{7′} radical,
where the substituent(s) R^{5′} is/are each independently of one another selected from:
cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)-alkylhydroxyimino, (C₁-C₆)alkoxyimino, (C₁-C₆)-alkyl-(C₁-C₆)-alkoxyimino, (C₁-C₆)-haloalkyl-(C₁-C₆)-alkoxyimino, (C₁-C₆)alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl and
R^{6′} and R^{7′} each independently of one another represent H, a (C₁-C₆)-alkyl, (C₁-C₆)-haloalkyl, (C₁-C₆)alkoxy-(C₁-C₆)-alkyl radical or unsubstituted phenyl radical or halogen-, (C₁-C₆)-alkyl-, (C₁-C₆)haloalkyl- or (C₁-C₆)-alkoxy-(C₁-C₆)-alkyl-substituted phenyl radical
or form a ring from the group pyrrolidine, morpholine, piperidine.

Very particular preference is given to an embodiment of formula (VIII) where (range of preference 5-2)

M′ represents a radical selected from the radicals of the formulae (IIa) to (IIf) where
in case (IIa) R^{2′} represents H, methyl or ethyl or optionally halogen-substituted phenyl and R^{3′} represents H, methyl, ethyl, isopropyl or halogen,
in case (IIb) R^{2′} represents H, methyl or ethyl,
in case (IIc) R^{2′} represents H or methyl and R^{3′} represents H, methyl or ethyl,
in case (IId) R¹ represents H or methyl and R^{3′} represents H or halogen,
in case (IIe) R^{2′} represents H, methyl, methoxy, halogen-substituted phenyl or halogen-substituted pyridyl and R³ represents H,
in case (IIf) R^{3′} represents H, methyl or ethyl and
Q′ represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituents R^{4′} are as defined in range of preference (2-2) or (3-2).

Even more preference is given to a configuration of formula (VIII) where (range of preference 6-2)

Q′ represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituent(s) R^{4′} is/are independently of one another selected from:
cyano, halogen, nitro, acetyl, (C₃-C₆)-cycloalkyl, (C₃-C₆)-cycloalkyloxy, (C₃-C₆)-cycloalkyl-(C₃-C₆)cycloalkyl, (C₁-C₆)-alkyl-(C₃-C₈)-cycloalkyl, halo-(C₃-C₆)-cycloalkyl, (C₁-C₆)-alkyl, (C₁-C₆)haloalkyl, (C₁-C₆)-cyanoalkyl, (C₁-C₆)-hydroxyalkyl, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)haloalkoxy, (C₁-C₆)-alkylthio, (C₁-C₆)-haloalkylthio, (C₁-C₆)-alkylthio-(C₁-C₆)-alkyl, (C₁-C₆)alkylsulphinyl, (C₁-C₆)-haloalkylsulphinyl, (C₁-C₆)-alkylsulphonyl, (C₁-C₆)-haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)alkylcarbonylamino, phenyl, halophenyl, phenoxy or halophenoxy;
D′ represents a (C₁-C₆)-alkyl, phenyl, pyridine, pyrimidine, pyrazole, triazole, thiazole, oxazole, thiadiazole, oxadiazole, thiophene, pyrrole, furan, tetrahydrofuran or dioxane radical which is unsubstituted or substituted by one or more radicals R^{5′},
where the substituent(s) R^{5′} is/are independently of one another selected from:
cyano, halogen, nitro, acetyl, hydroxy, carboxy, amino, (C₃-C₆)-cycloalkyl, (C₃-C₆)-cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)-hydroxyalkyl, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)alkyl, (C₁-C₆)-alkylsulphinyl, (C₁-C₆)-haloalkylsulphinyl, (C₁-C₆)-alkylsulphonyl, (C₁-C₆)haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino or (1-pyrazolyl) (C₁-C₃)-alkyl and
R^{6′} and R^{7′} each independently of one another represent H, (C₁-C₆)-alkyl, phenyl, alkoxyphenyl or halophenyl or form a ring from the group pyrrolidine, morpholine, piperidine.

Finally particular preference is given to a configuration of formula (VIII) where (range of preference 7-2)

M′ represents a radical selected from the radicals of the formulae (Va-Vz):

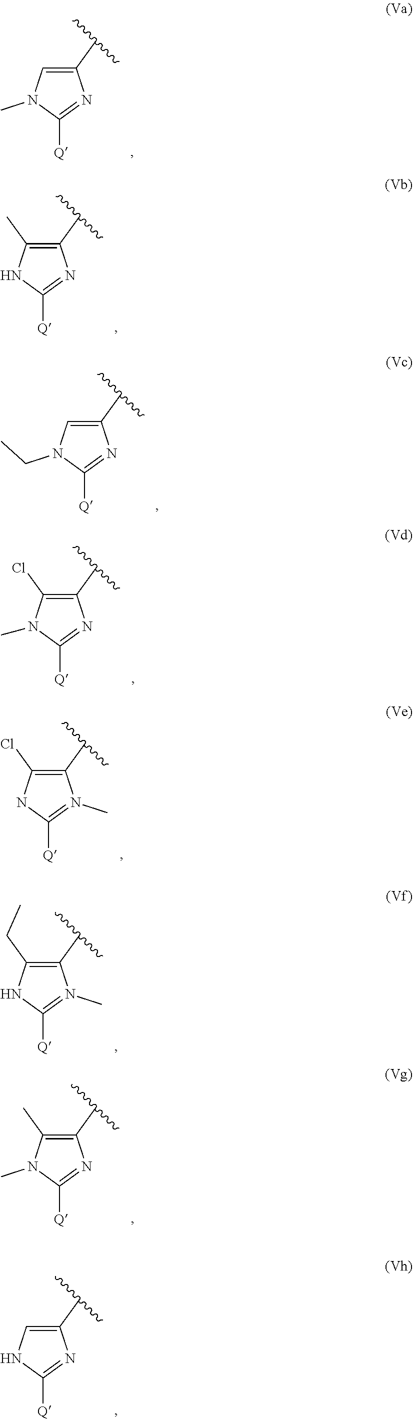

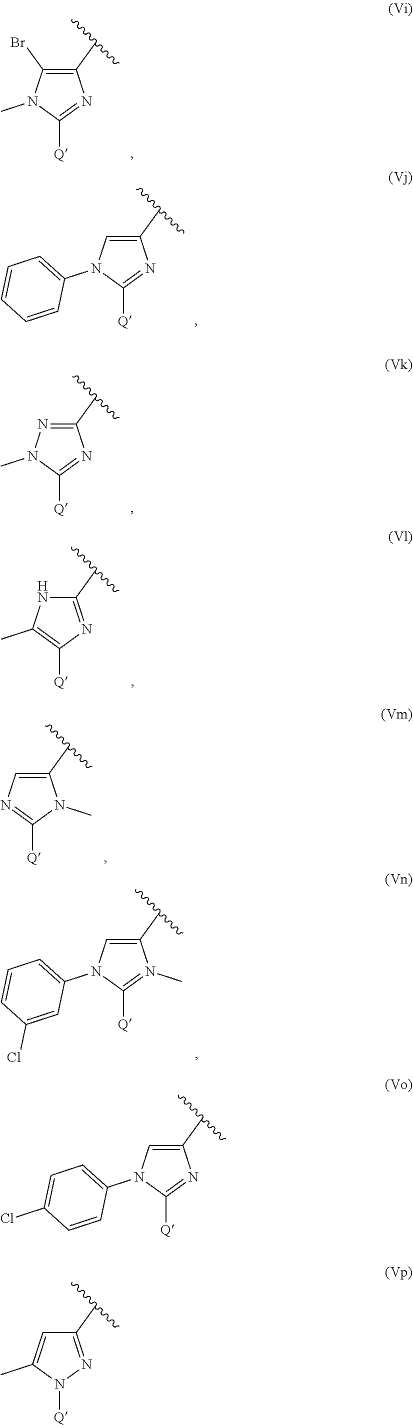

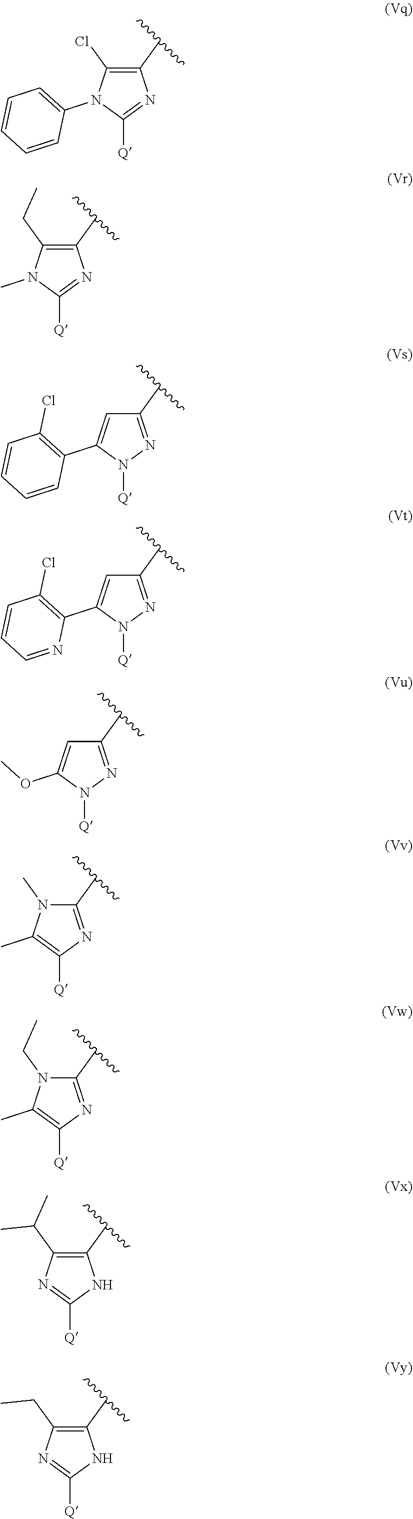

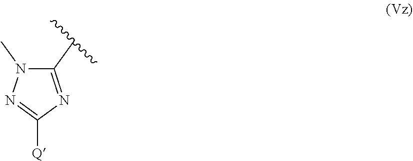

Q′ represents a radical selected from the radicals of the formulae (VIa-VIz and VIa1-VIa30):

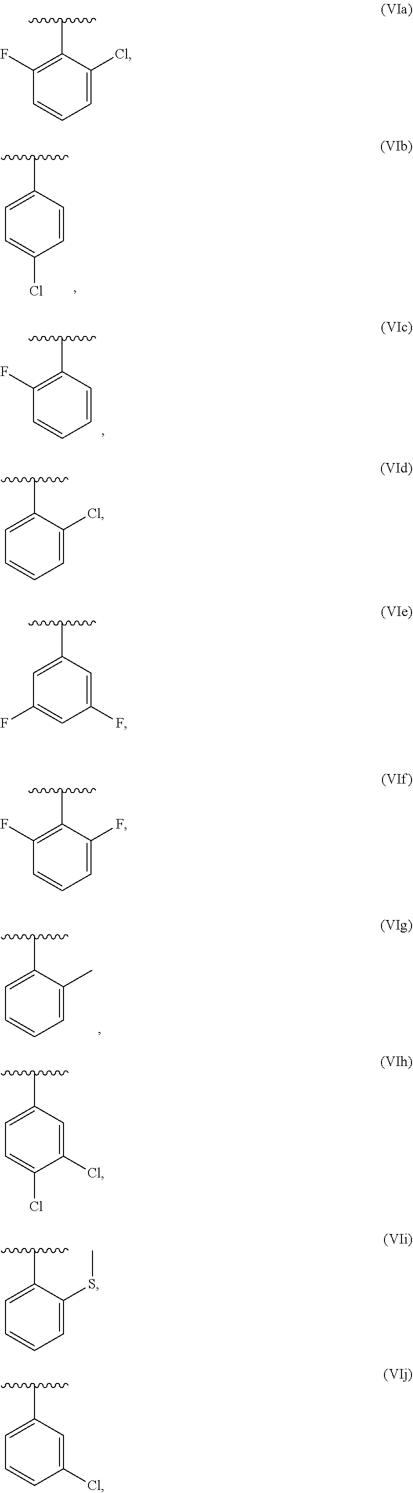

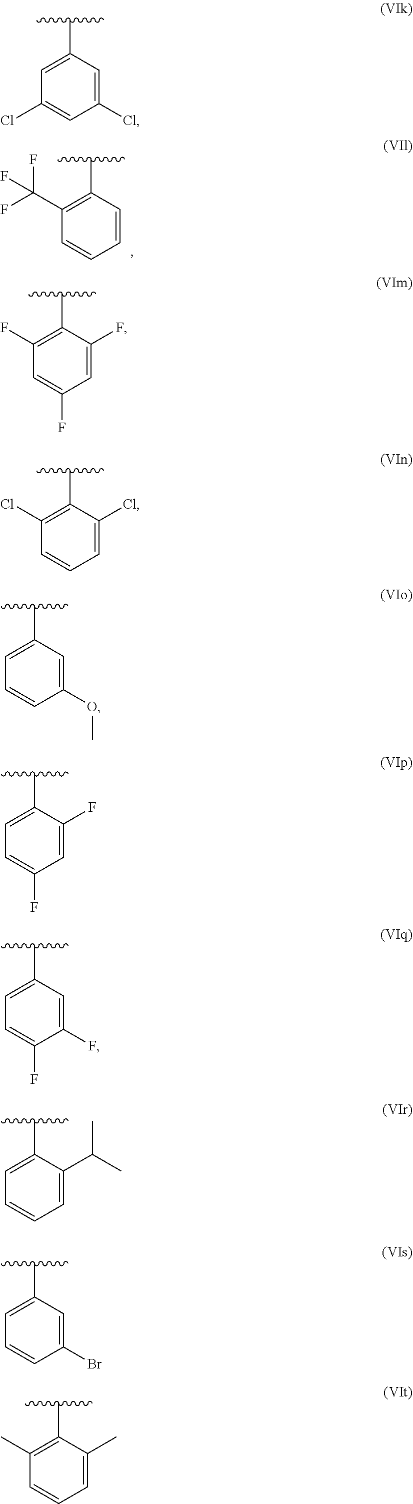

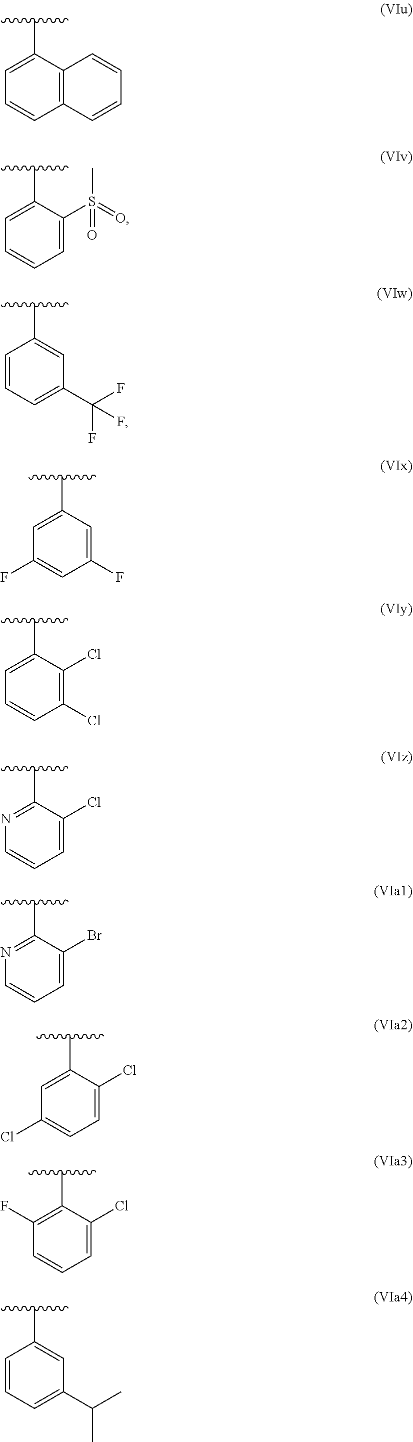

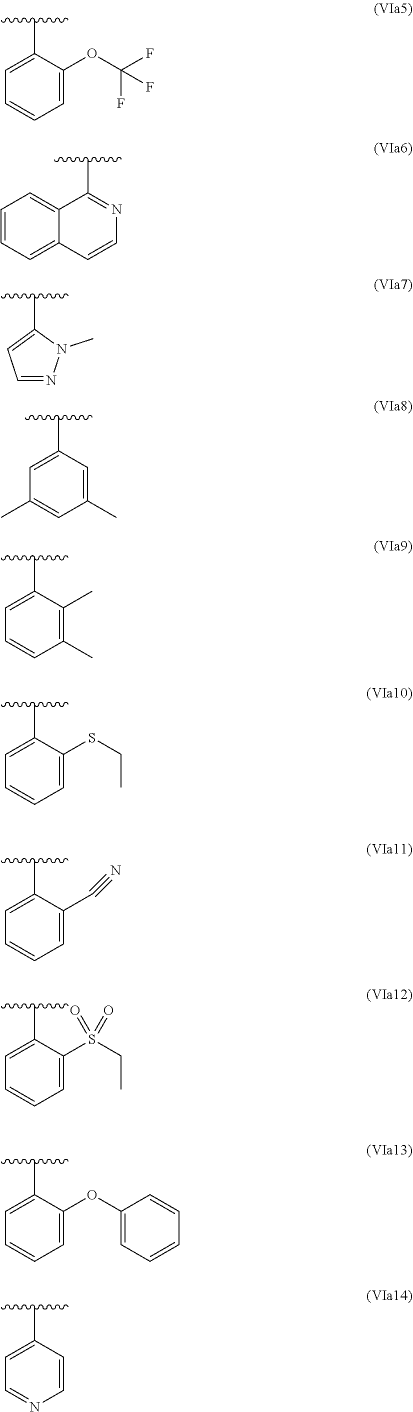

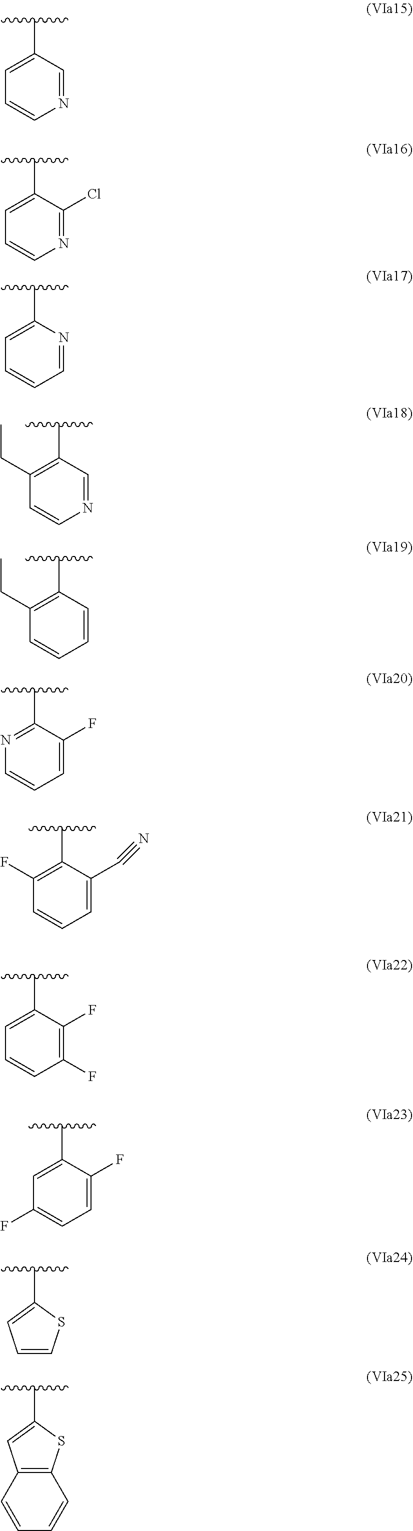

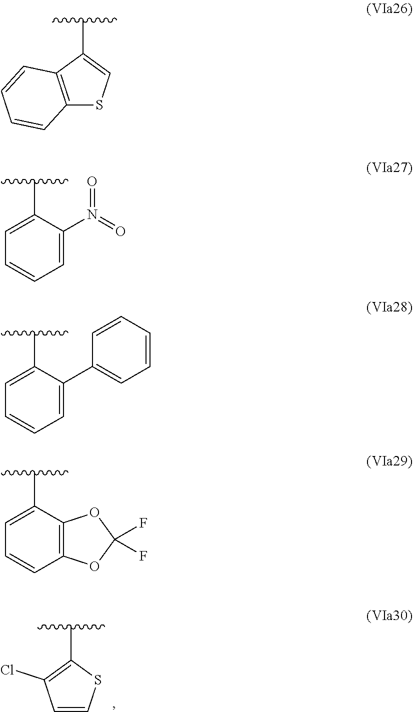

D′ represents a radical selected from the radicals of the formulae (VII1-VII192)

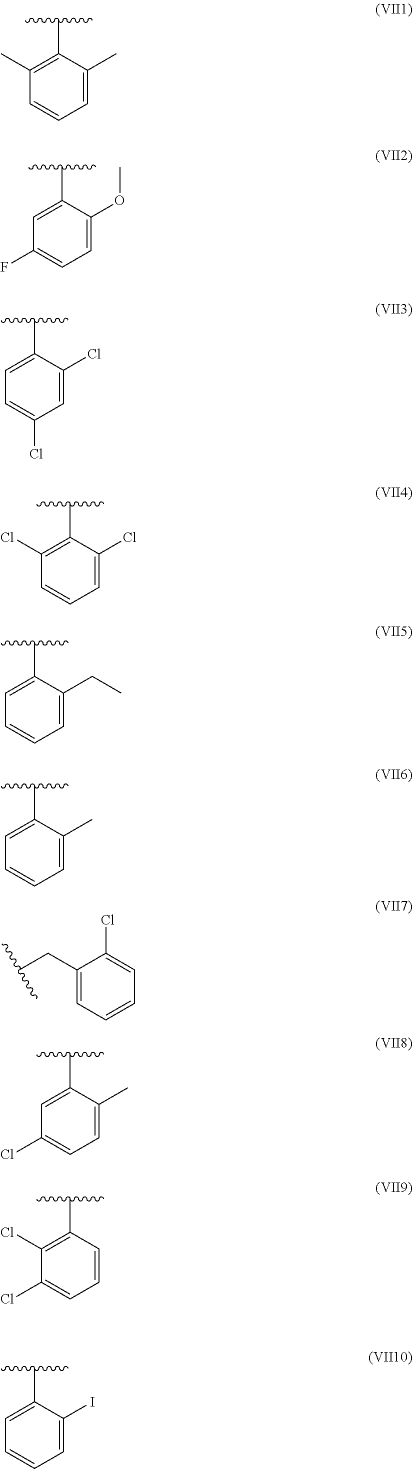

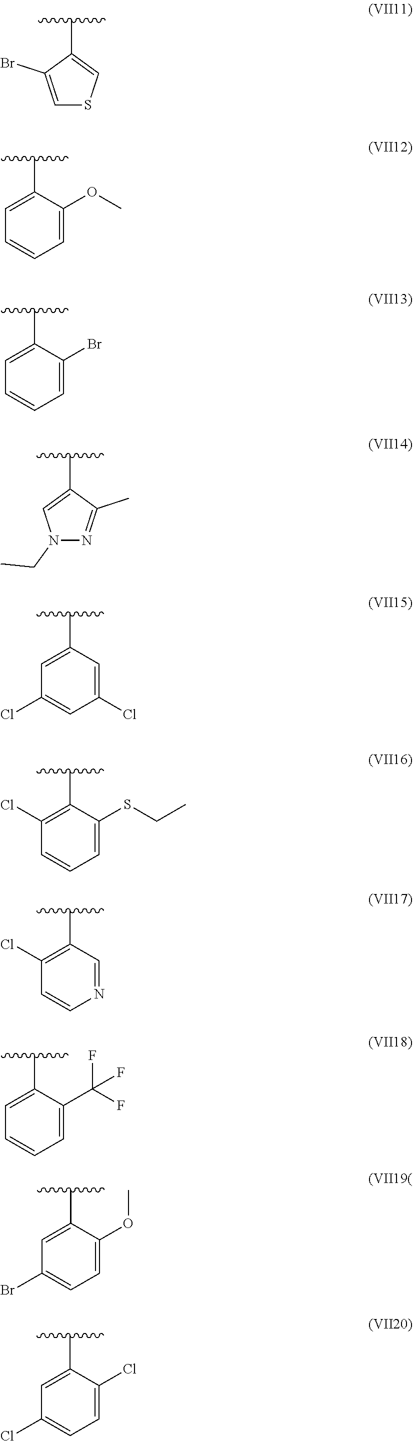

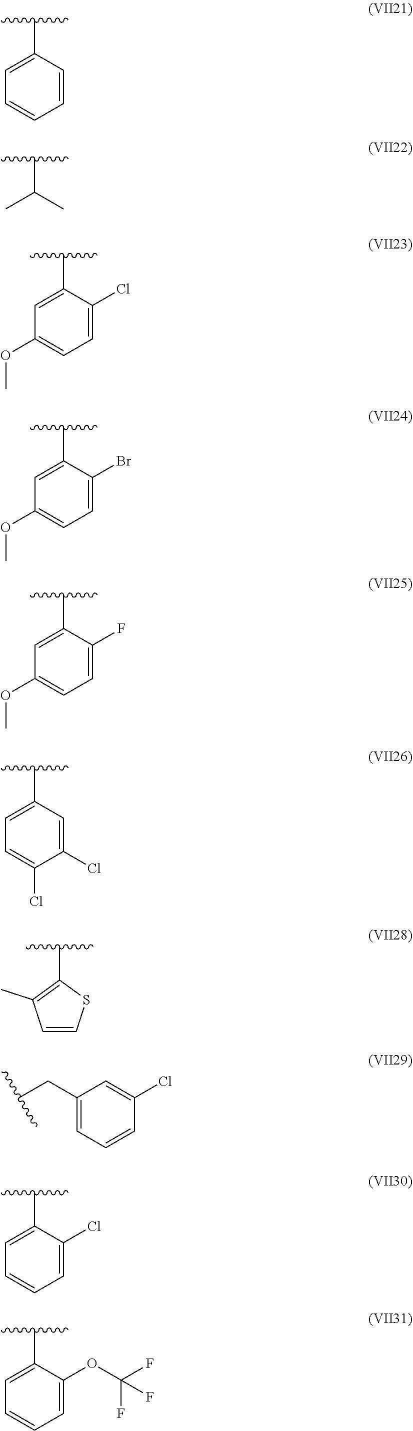

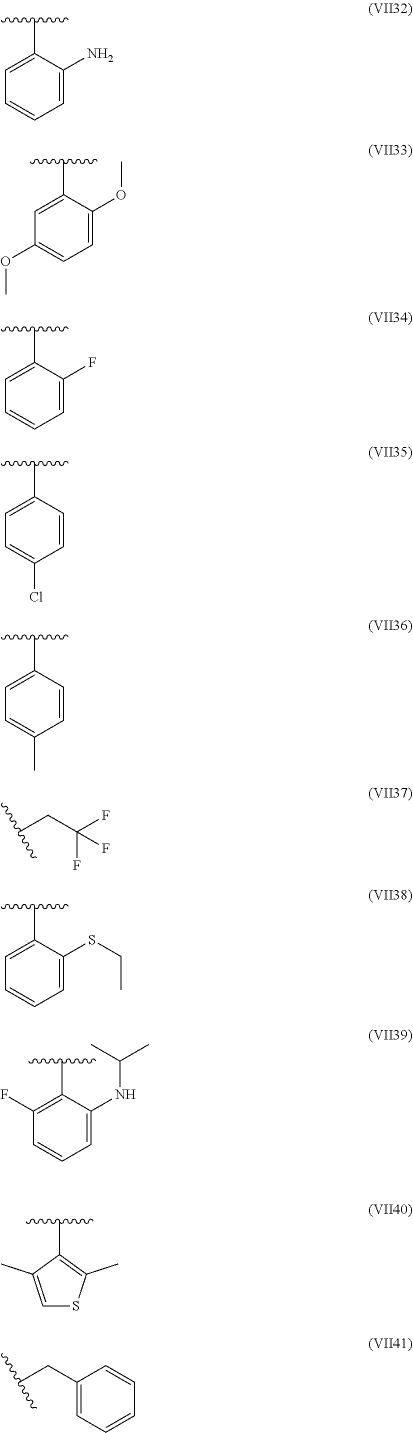

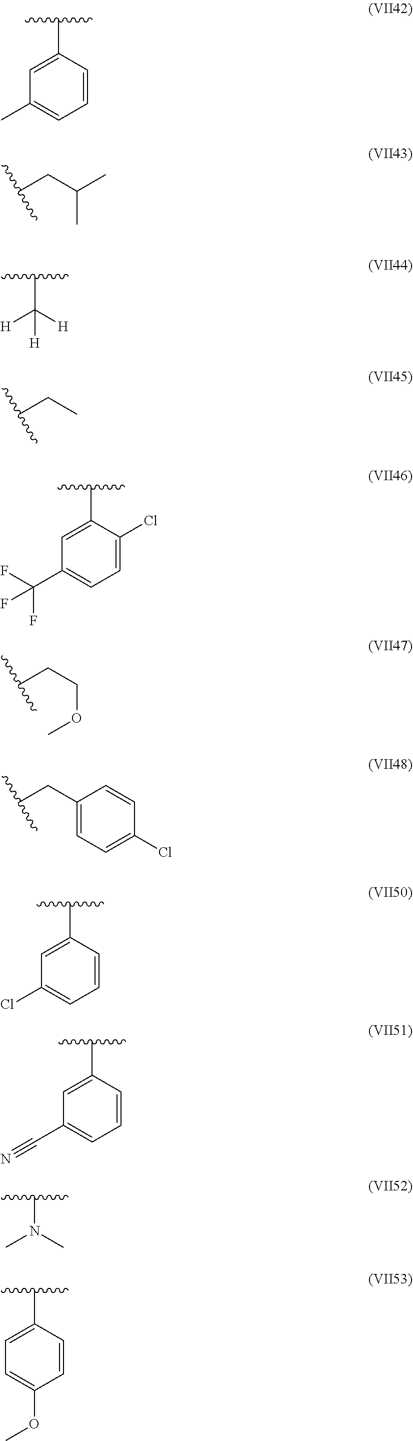

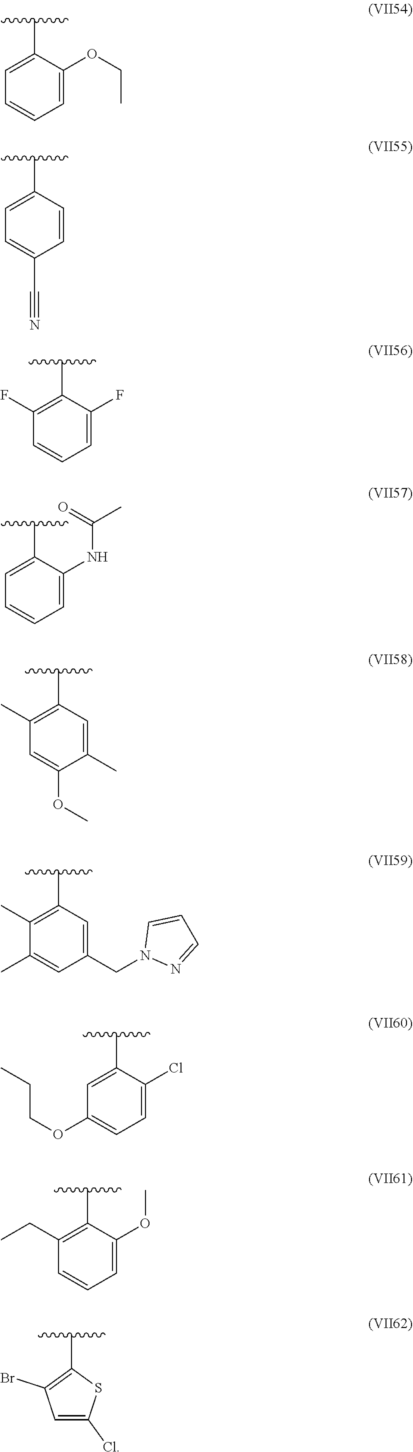

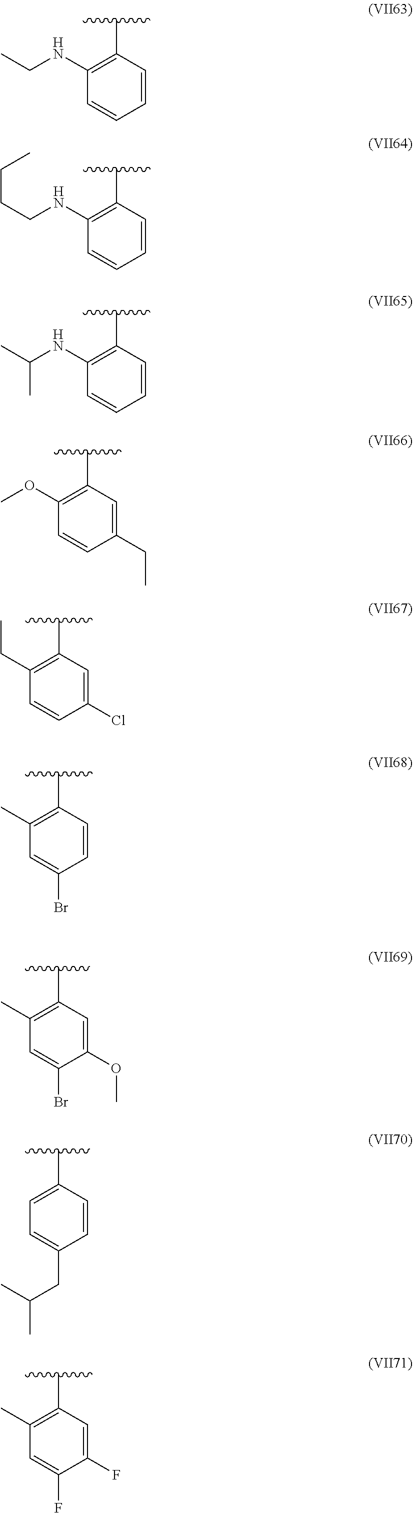

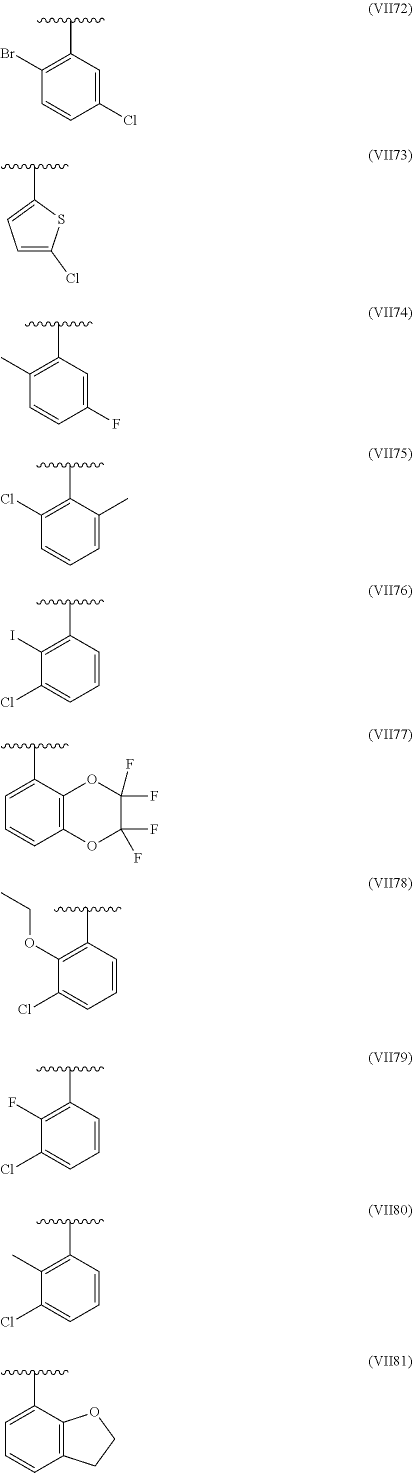

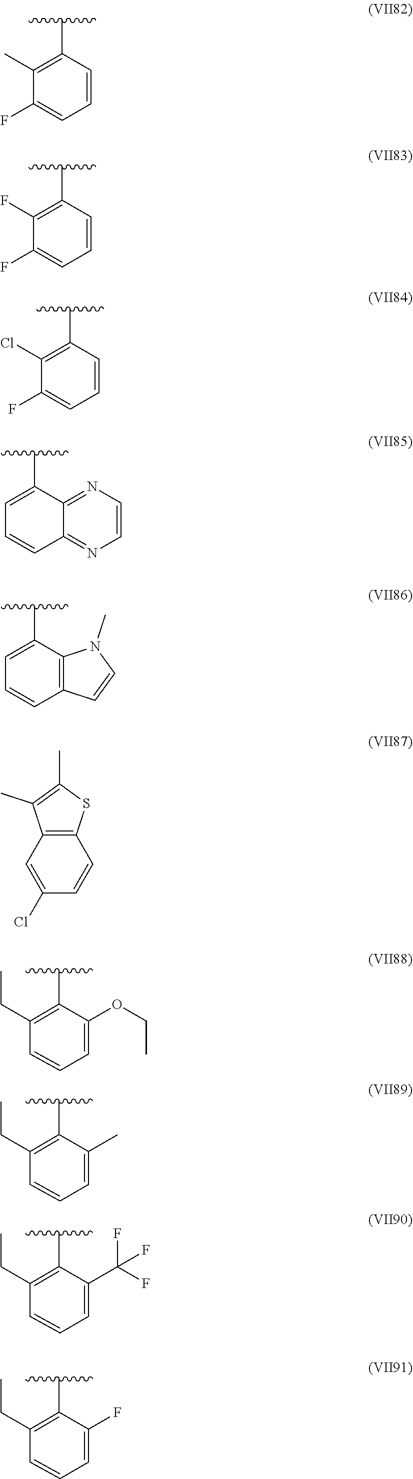

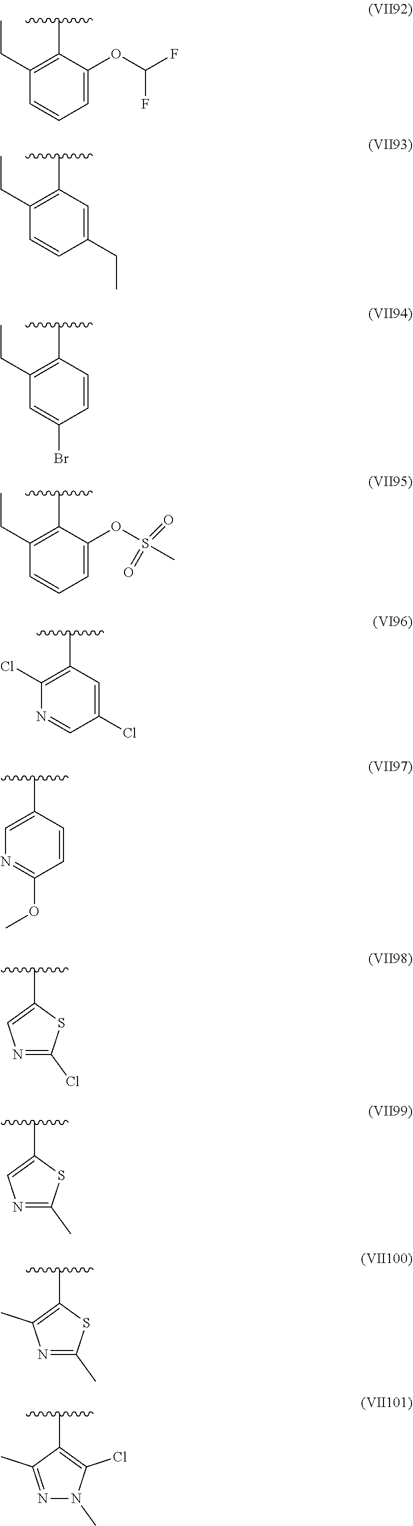

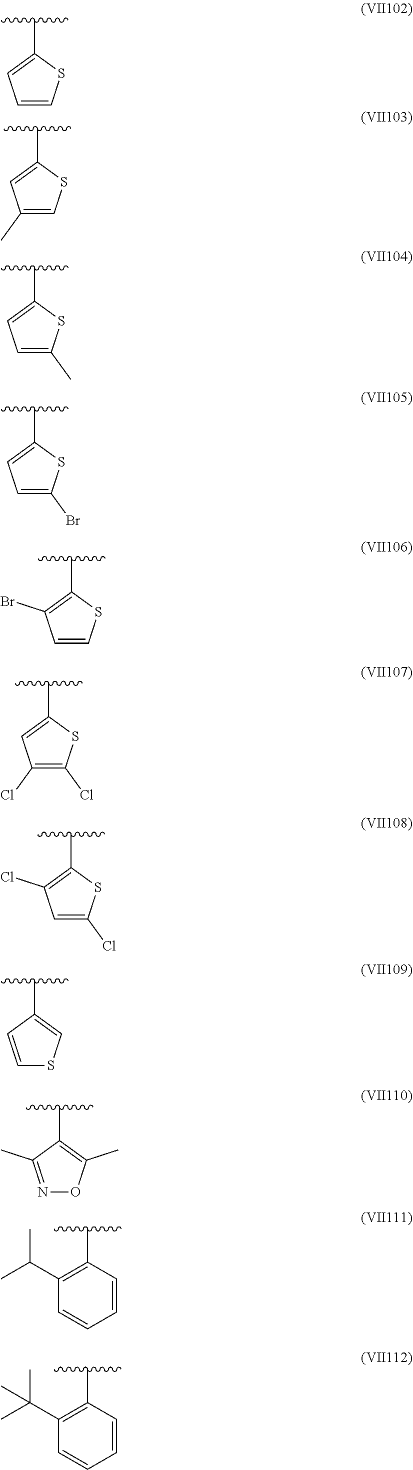

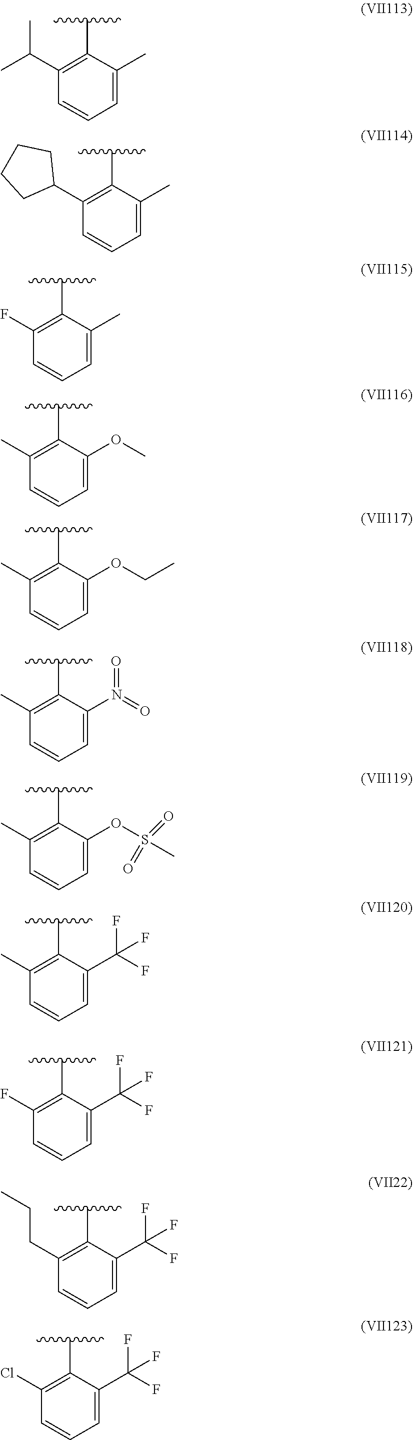

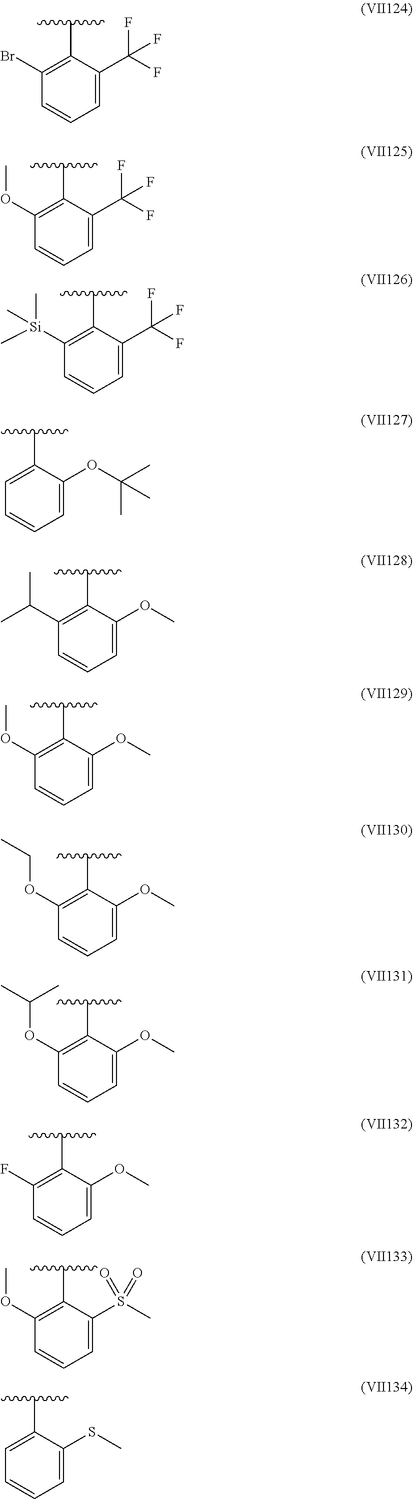

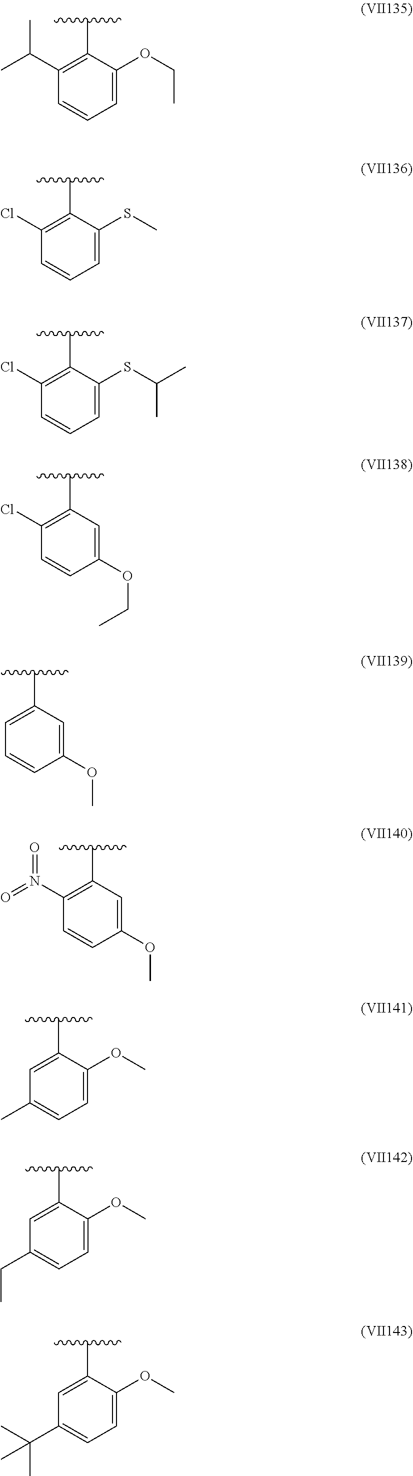

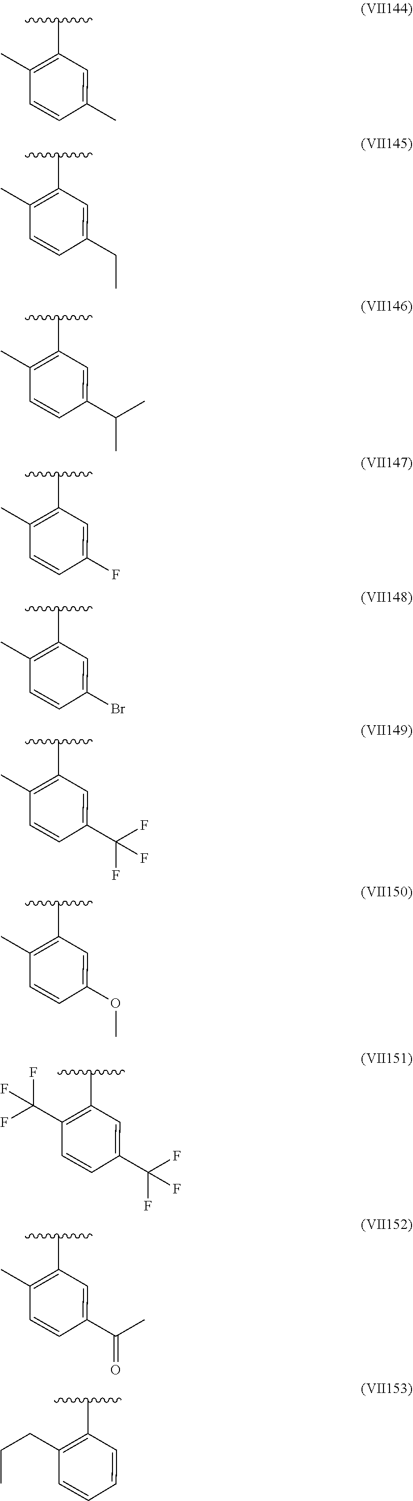

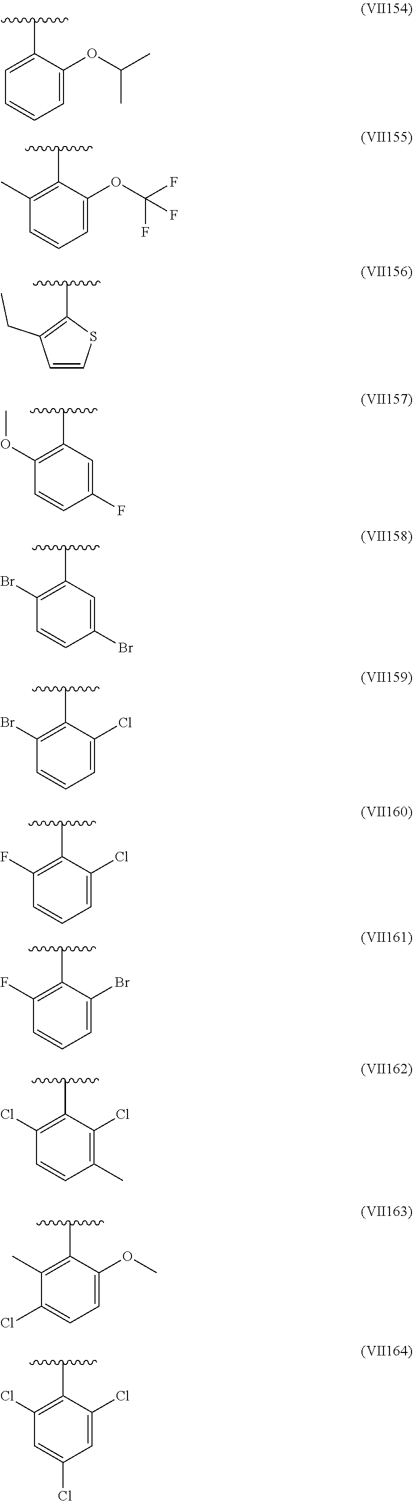

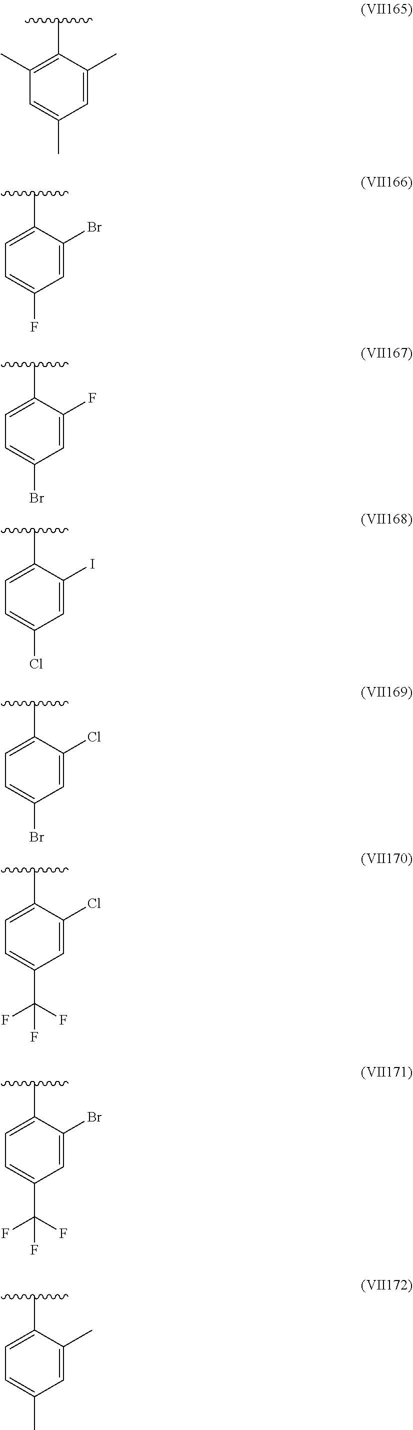

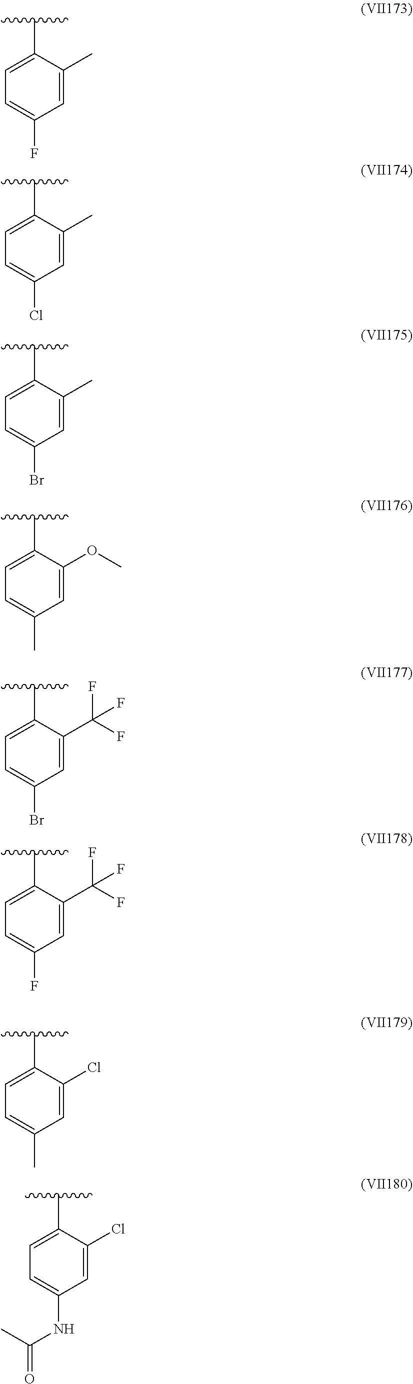

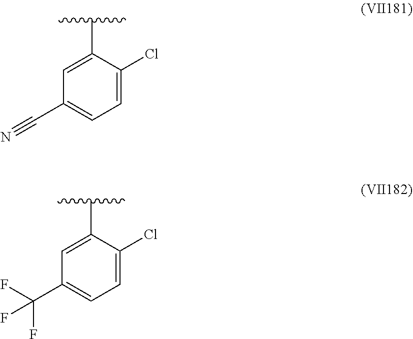 [SMILES: *c1cc(C#N)ccc1Cl ; *c1cc(C(F)(F)F)ccc1Cl]

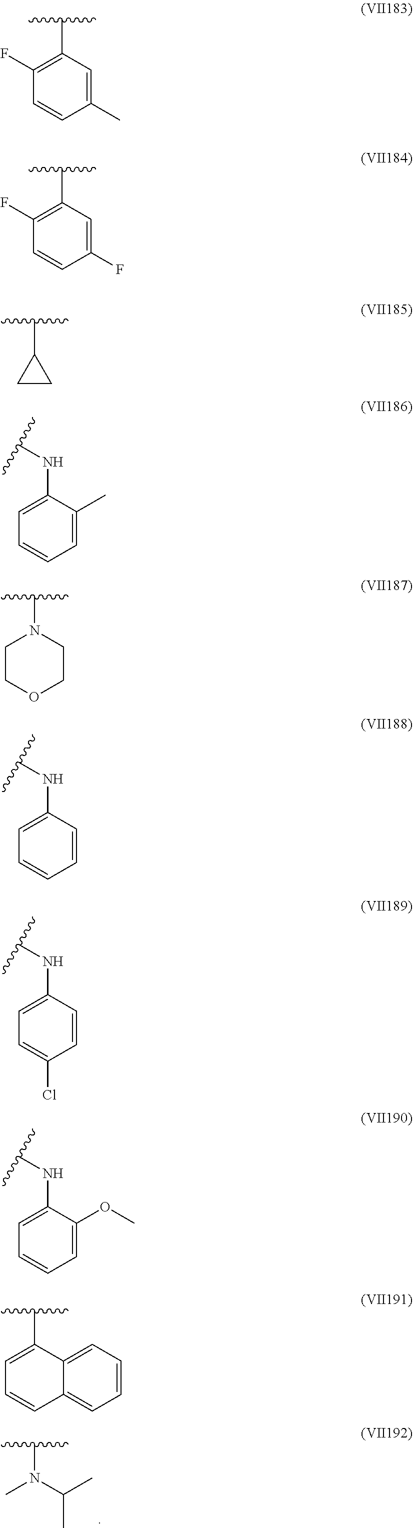

The invention furthermore provides intermediates of the formulae XIa-XIq:

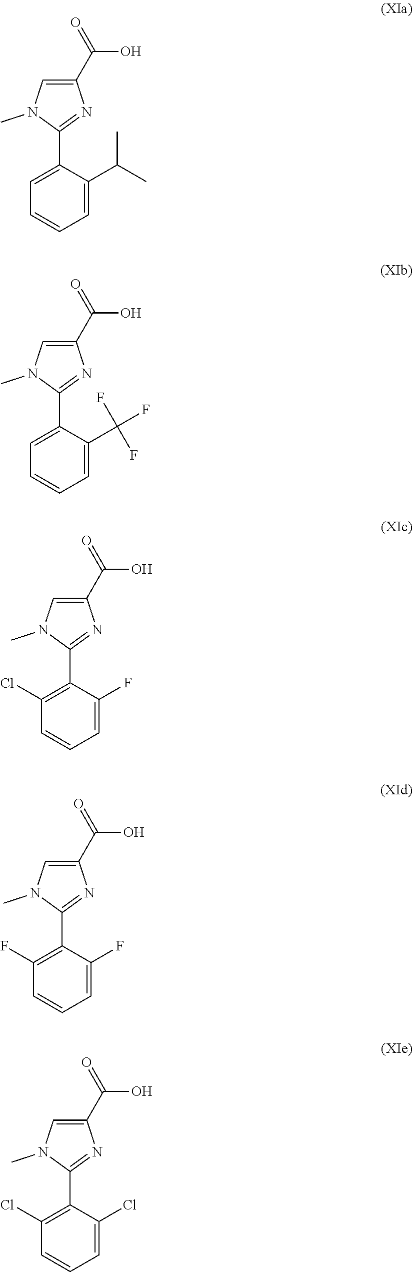

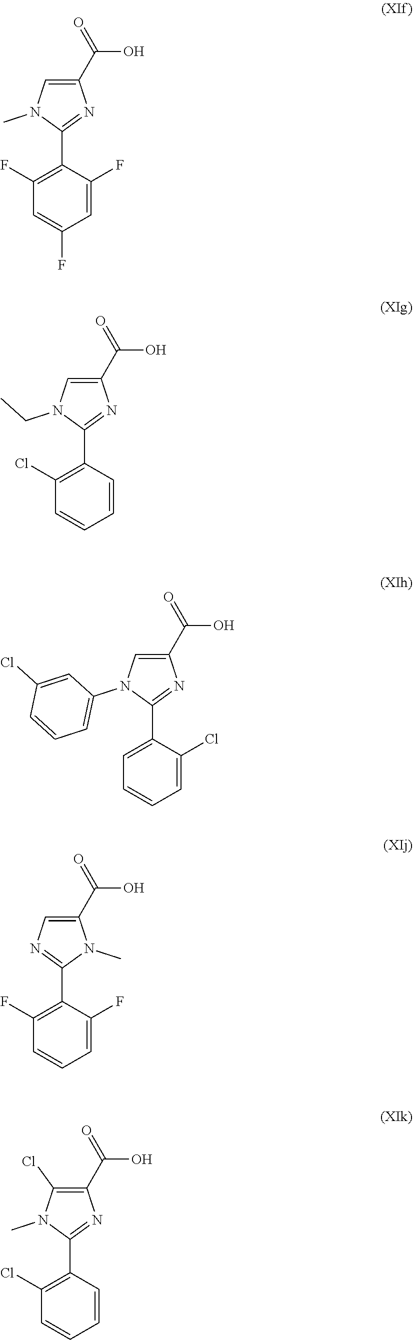 [SMILES: Cn1cc(C(=O)O)nc1-c1c(F)cc(F)cc1F ; CCn1cc(C(=O)O)nc1-c1ccccc1Cl ; O=C(O)c1cn(-c2cccc(Cl)c2)c(-c2ccccc2Cl)n1 ; Cn1c(C(=O)O)cnc1-c1c(F)cccc1F ; Cn1c(-c2ccccc2Cl)nc(C(=O)O)c1Cl]

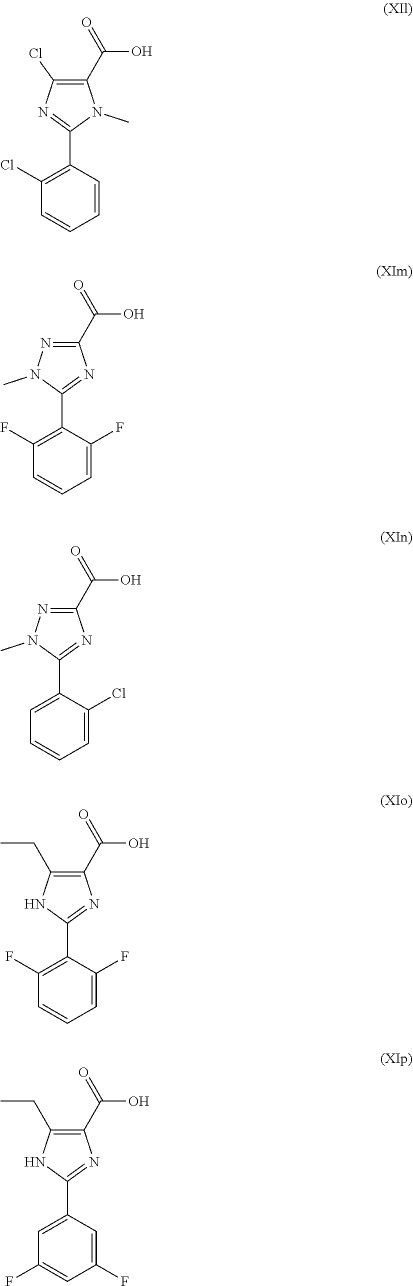 [SMILES: Cn1c(-c2ccccc2Cl)nc(Cl)c1C(=O)O ; Cn1nc(C(=O)O)nc1-c1c(F)cccc1F ; Cn1nc(C(=O)O)nc1-c1ccccc1Cl ; CCc1[nH]c(-c2c(F)cccc2F)nc1C(=O)O ; CCc1[nH]c(-c2cc(F)cc(F)c2)nc1C(=O)O]

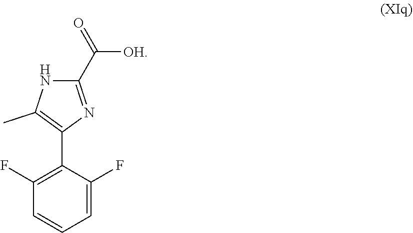 [SMILES: Cc1[nH]c(C(=O)O)nc1-c1c(F)cccc1F]

Further ranges of preference of the invention are listed below:

Use of a compound of the formula (I)

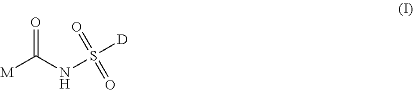

in which
M represents a radical selected from the formulae (IIa-IIe):

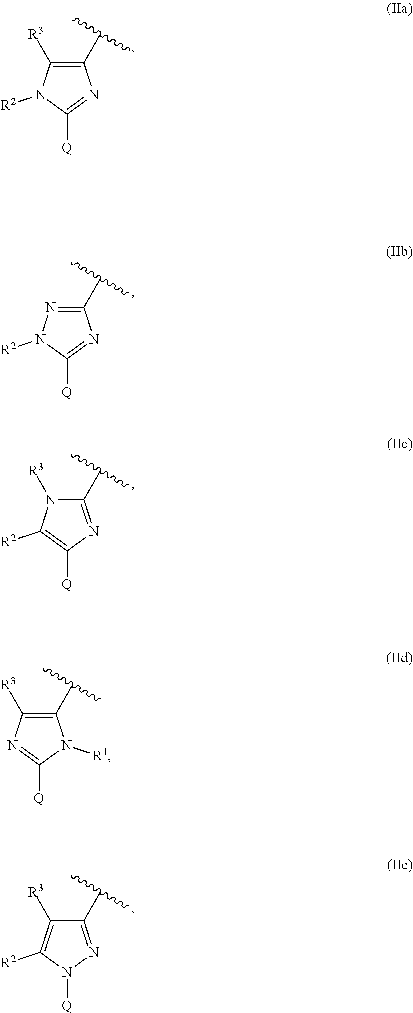

where
R¹, R², R³ each independently of one another represent H or a substituted or unsubstituted alkyl, cycloalkyl, alkenyl, cycloalkenyl, cycloheteroalkyl, aryl, heteroaryl radical, where in cases (IIc) and (IIe) R² may additionally represent a halogen radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
for controlling animal pests.

Preference is given to an embodiment where

R¹, R², R³ each independently of one another represent H or a substituted or unsubstituted C₁-C₄-alkyl, C₃-C₆-cycloalkyl, C₂-C₄-alkenyl or phenyl radical, where in cases (IIc) and (IIe) R² may additionally represent a halogen radical and where in cases (IIa), (IId) and (IIe) R³ may additionally represent a halogen radical;
Q represents a substituted or unsubstituted phenyl, naphthyl or heteroaryl radical which may contain one to three heteroatoms from the group consisting of oxygen, sulphur, nitrogen; but, in case (IIe), does not represent 2-pyrimidinyl;
D represents a substituted or unsubstituted alkyl, heteroalkyl, cycloalkyl, heteroaryl, aryl or phenyl(C₁-C₈)-alkyl radical or a substituted or unsubstituted nitrogen radical.

More preference is given to an embodiment where

Q represents a phenyl, naphthyl, pyridyl, pyrimidinyl, thiophene or pyrazole radical which is unsubstituted or substituted by one or more radicals R⁴, but, in case (IIe), does not represent 2-pyrimidinyl;
where the substituent(s) R⁴ each independently of one another represent(s):
hydrogen, cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino); and/or
aryl or hetaryl, optionally mono- or polysubstituted by identical or different substituents, where (in the case of hetaryl) optionally at least one carbonyl group may be present and/or where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino;
D represents a radical, unsubstituted or substituted by one or more radicals R⁵, from the group consisting of C₁-C₆-alkyl, phenyl, phenyl-(C₁-C₂)-alkyl and heteroaryl which may contain one to three heteroatoms from the group consisting of oxygen, sulphur, nitrogen, or represents an NR⁶R⁷ radical,
where the substituent(s) R⁵ each independently of one another represent(s):
hydrogen, cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino), (1-pyrazolyl)-(C₁-C₃)-alkyl,
and/or
aryl or hetaryl, mono- or polysubstituted by identical or different substituents, where (in the case of hetaryl) optionally at least one carbonyl group may be present and/or where possible substituents in each case are as follows: cyano, carboxyl, halogen, nitro, acetyl, hydroxy, amino, SCN, tri-(C₁-C₆)alkylsilyl, (C₃-C₈)-cycloalkyl, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino;
and where R⁶ and R⁷ each independently of one another represent H, C₁-C₆-alkyl or a substituted or unsubstituted phenyl radical or R⁶ and R⁷ together may form an unsubstituted or substituted 4- to 8-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R⁵.

Even more preferably

the substituent(s) R⁴ each independently of one another represent(s):
hydrogen, cyano, halogen, nitro, acetyl, hydroxy, carboxy, amino, tri-(C₁-C₆)-alkylsilyl, (C₃-C₈)cycloalkyl, (C₃-C₈)-cycloalkyl-(C₃-C₈)-cycloalkyl, (C₁-C₆)-alkyl-(C₃-C₈)-cycloalkyl, halo-(C₃-C₈)cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)alkenyl, (C₂-C₆)-haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)haloalkylthio, (C₁-C₆)-alkoxy-(C₁-C₆)-alkylthio, (C₁-C₆)-alkylthio-(C₁-C₆)-alkyl, (C₁-C₆)alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino;
the substituent(s) R⁵ each independently of one another represent(s):
hydrogen, cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, (1-pyrazolyl)-(C₁-C₃)-alkyl;
R⁶ and R⁷ each independently of one another represent H, a C₁-C₆-alkyl, (C₁-C₆)-haloalkyl, (C₁-C₆)alkoxy-(C₁-C₆)-alkyl radical or an unsubstituted phenyl radical or a phenyl radical substituted by halogen, C₁-C₆-alkyl, (C₁-C₆)-haloalkyl, (C₁-C₆)-alkoxy-(C₁-C₆)-alkyl,
or
R⁶ und R⁷ together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R⁵.

Special preference is given to an embodiment where

in case (IIa) R², R³ each independently of one another represent H or a substituted or unsubstituted C₁-C₄-alkyl, C₃-C₆-cycloalkyl or phenyl radical, where R³ may additionally represent a halogen radical,
in case (IIb) R² represents H or a substituted or unsubstituted C₁-C₄-alkyl or C₃-C₆-cycloalkyl radical,
in case (IIc) R², R³ each independently of one another represent H or a substituted or unsubstituted C₁-C₄-alkyl, C₃-C₆-cycloalkyl or phenyl radical,
in case (IId) R¹, R³ each independently of one another represent H or a substituted or unsubstituted C₁-C₄-alkyl, C₃-C₆-cycloalkyl radical,
in case (IIe) R², R³ each independently of one another represent H or halogen or a substituted or unsubstituted C₁-C₄-alkyl, C₃-C₆-cycloalkyl or phenyl radical,
D represents a C₁-C₆-alkyl radical or phenyl radical or pyridine, pyrimidine, pyrazole, triazole, thiazole, oxazole, thiadiazole, oxadiazole, thiophene, pyrrole, furan, tetrahydrofuran, dioxane radical which is unsubstituted or substituted by one or more radicals R^{5′}, or represents an NR^{6′}R^{7′} radical, where
R^{6′} and R^{7′} each independently of one another represent H, a C₁-C₆-alkyl, (C₁-C₆)-haloalkyl, (C₁-C₆)alkoxy-(C₁-C₆)-alkyl radical or an unsubstituted phenyl radical or a phenyl radical substituted by halogen, C₁-C₆-alkyl, (C₁-C₆)-haloalkyl, (C₁-C₆)-alkoxy-(C₁-C₆)-alkyl,
or form a ring from the group pyrrolidine, morpholine, piperidine.

More preference still is given to an embodiment where

M represents a radical selected from the radicals of the formulae (IVa), (IVb), (IVc) (IVd), (IVe)

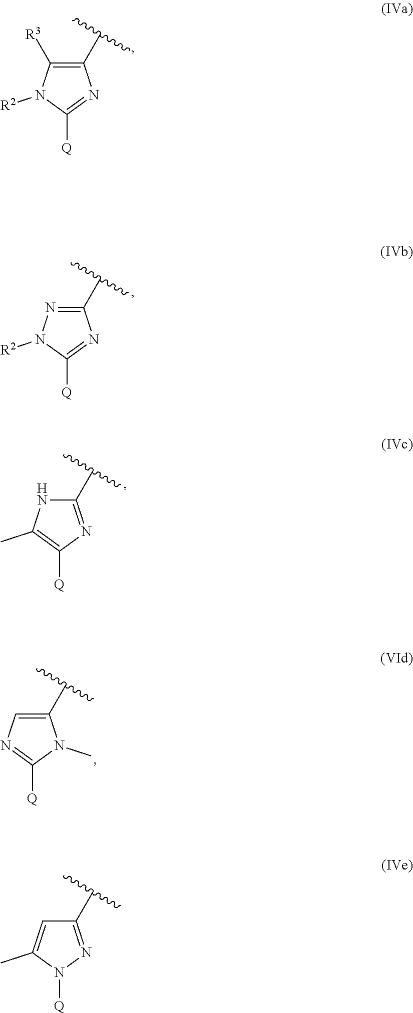

where
in case (IVa) R² represents H, methyl or ethyl or halogen-substituted phenyl and R³ represents H, methyl, ethyl or halogen, and
in case (IVb) R² represents H, methyl or ethyl.

Very particular preference is given to an embodiment where

Q represents a phenyl, naphthyl, pyridyl, pyrimidinyl, thiophene or pyrazole radical which is unsubstituted or substituted by one or more radicals R⁴, but, in case (IVe), does not represent 2-pyrimidinyl,
where the substituent(s) R⁴ independently of one another represent(s):
hydrogen, cyano, halogen, nitro, acetyl, (C₃-C₆)-cycloalkyl, (C₃-C₆)-cycloalkyloxy, (C₃-C₆)cycloalkyl-(C₃-C₆)-cycloalkyl, (C₁-C₆)-alkyl-(C₃-C₈)-cycloalkyl, halo-(C₃-C₆)-cycloalkyl, (C₁-C₆)alkyl, (C₁-C₆)-haloalkyl, (C₁-C₆)-cyanoalkyl, (C₁-C₆)-hydroxyalkyl, (C₁-C₆)-alkoxycarbonyl-(C₁-C₆)alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkylsulfonyl, (C₁-C₆)-haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino,
where the substituent(s) R⁵ independently of one another represent(s):
hydrogen, cyano, halogen, nitro, acetyl, hydroxy, carboxy, amino, (C₃-C₆)-cycloalkyl, (C₃-C₆)cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)cycloalkyl, (C₁-C₆)-alkyl, (C₁-C₆)-haloalkyl, (C₁-C₆)-cyanoalkyl, (C₁-C₆)-hydroxyalkyl, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)-haloalkylsulphinyl, (C₁-C₆)alkylsulphonyl, (C₁-C₆)-haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino), (1-pyrazolyl)-(C₁-C₃)-alkyl,
R⁶ and R⁷ each independently of one another represent H, C₁-C₆-alkyl or form a ring from the group pyrrolidine, morpholine, piperidine.

Even more preferably

M represents a radical selected from the radicals of the formulae (Va-Vr):

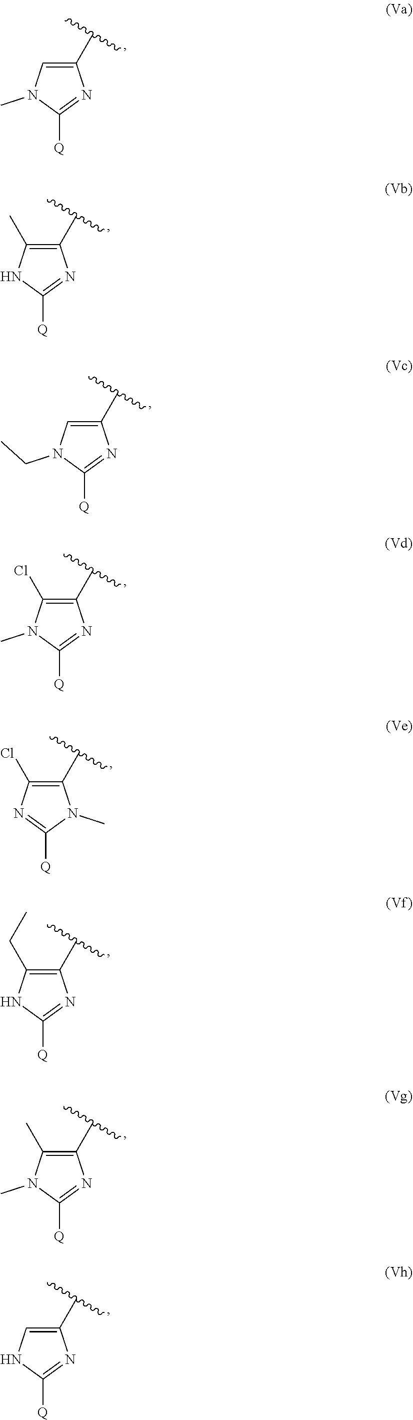

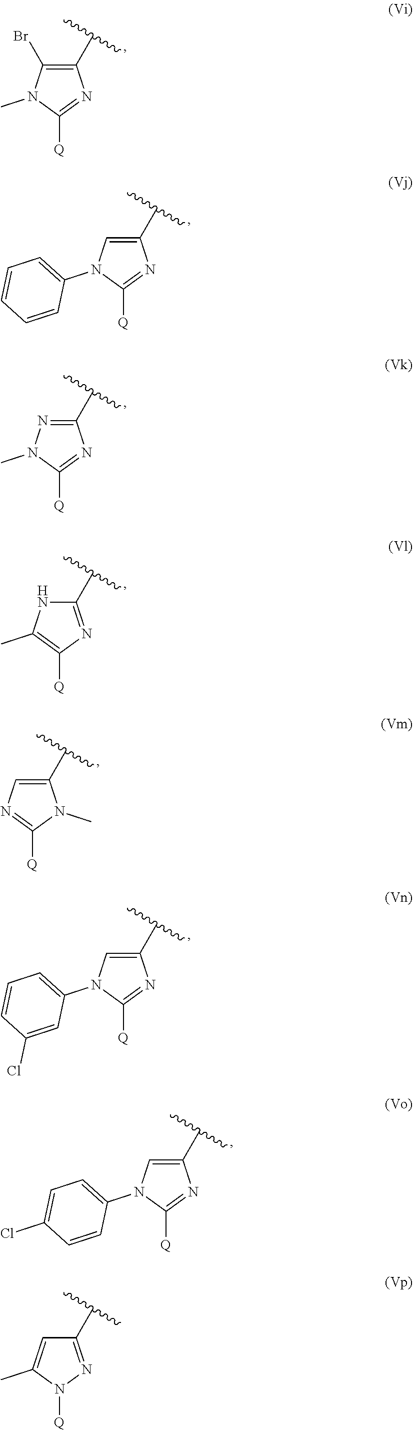

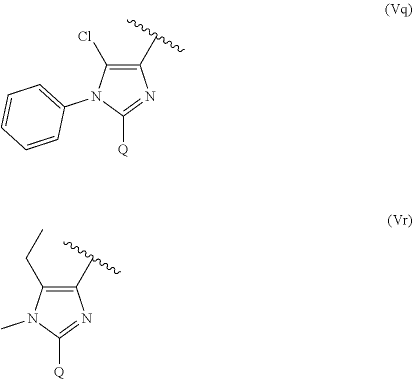

Q represents a radical selected from the radicals of the formulae (VIa-VIv):

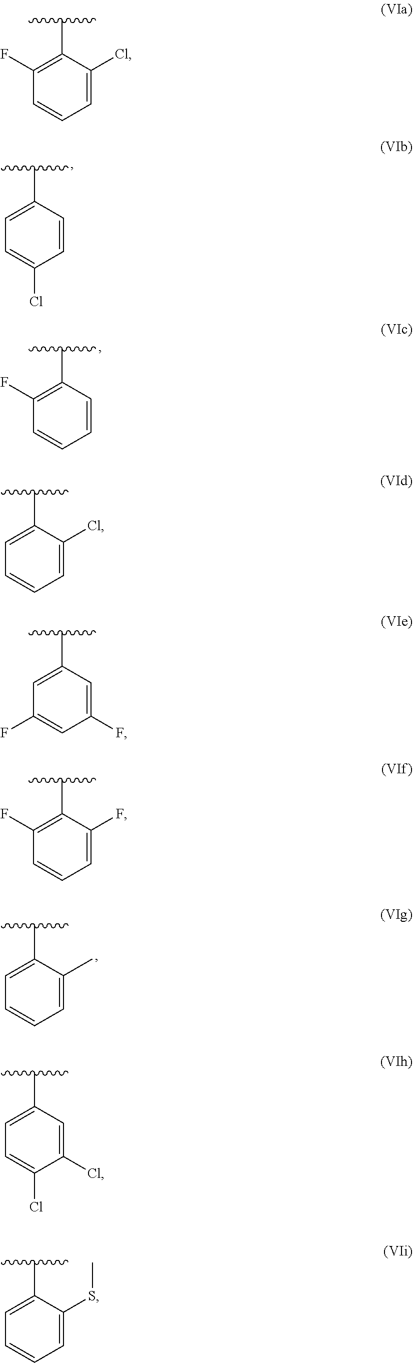

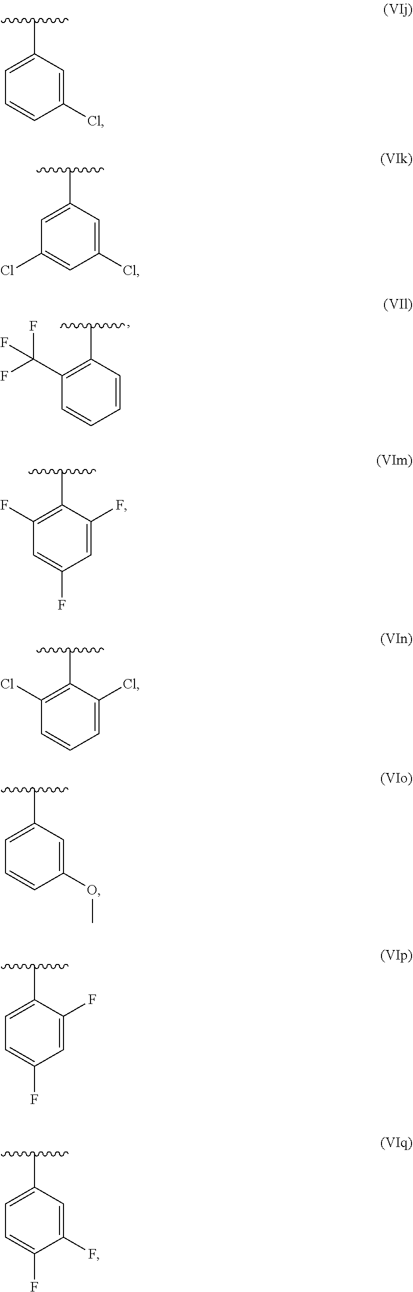

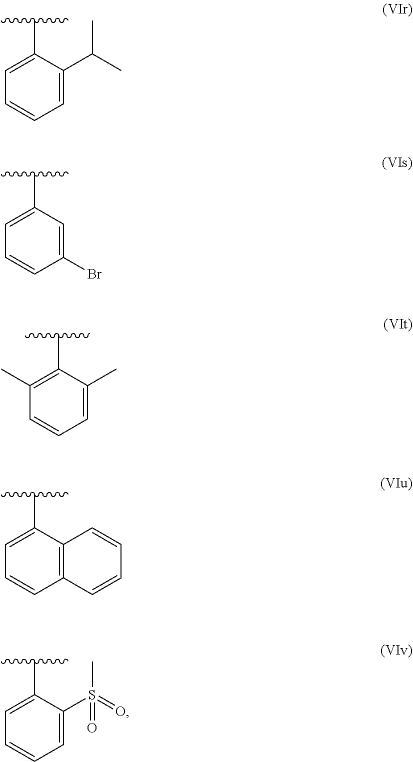

D represents a radical selected from the radicals of the formulae (VII1-VII62)

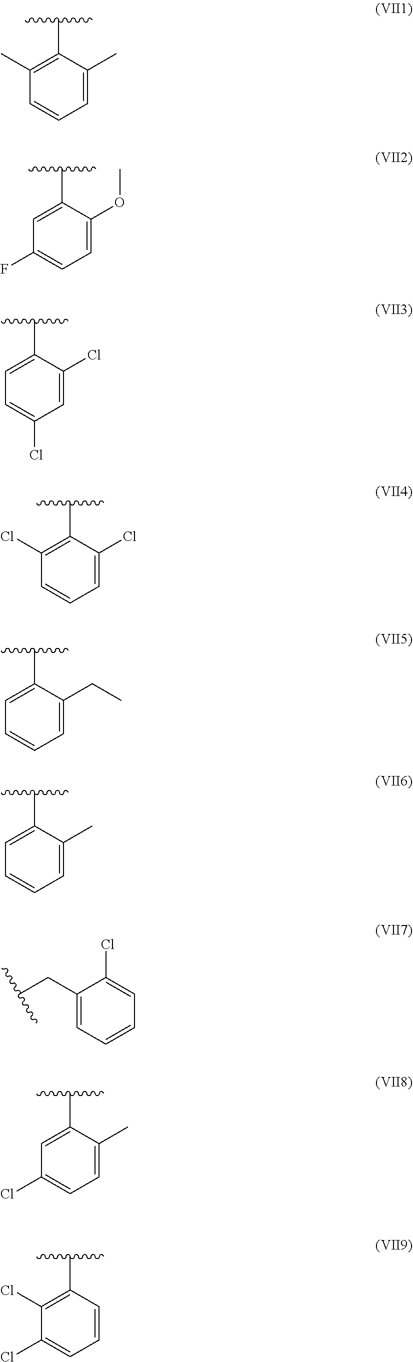

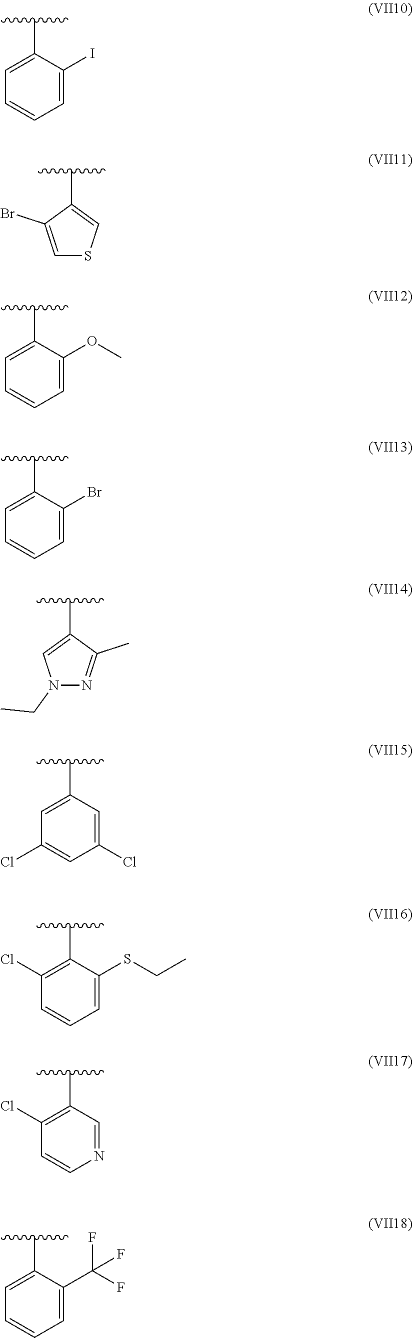

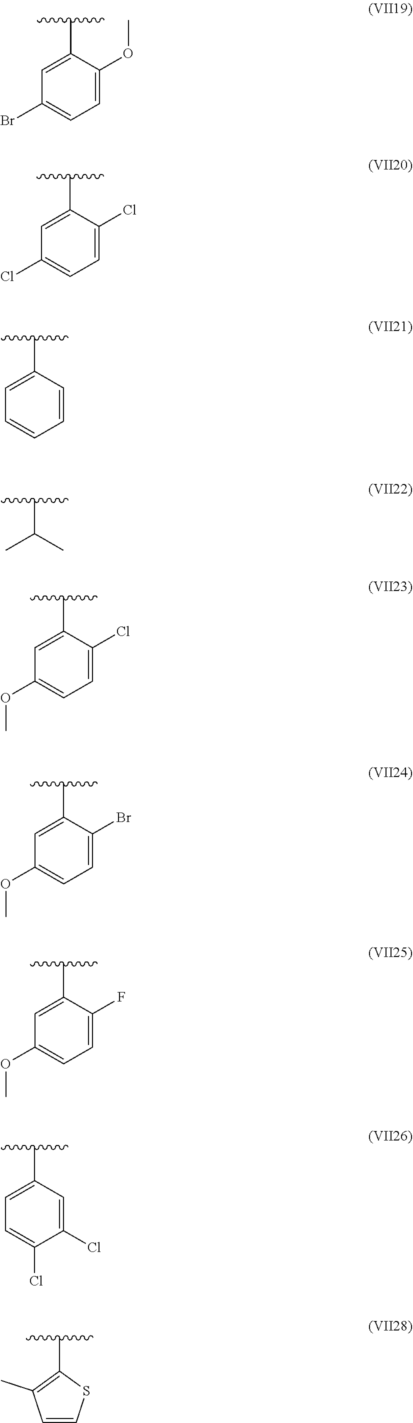

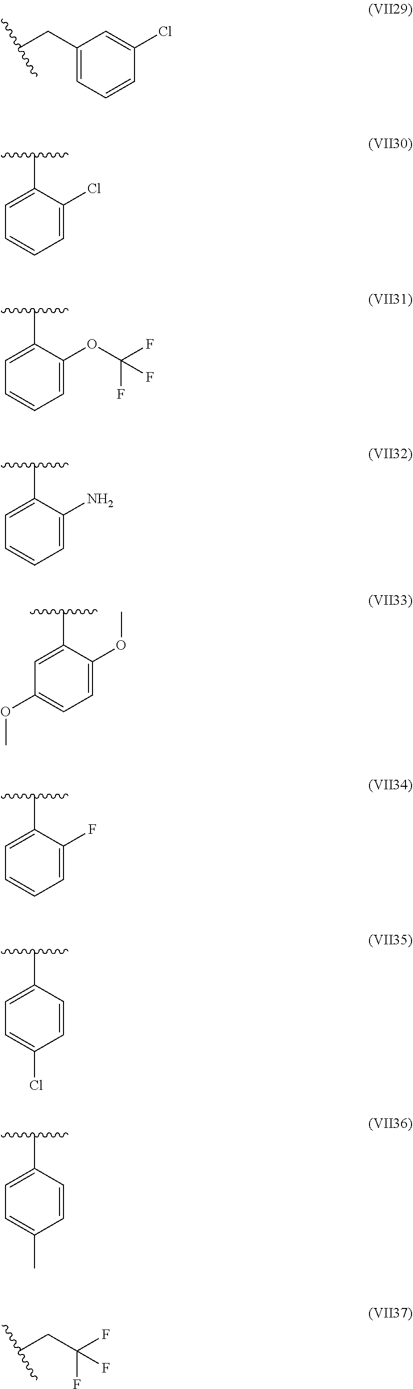

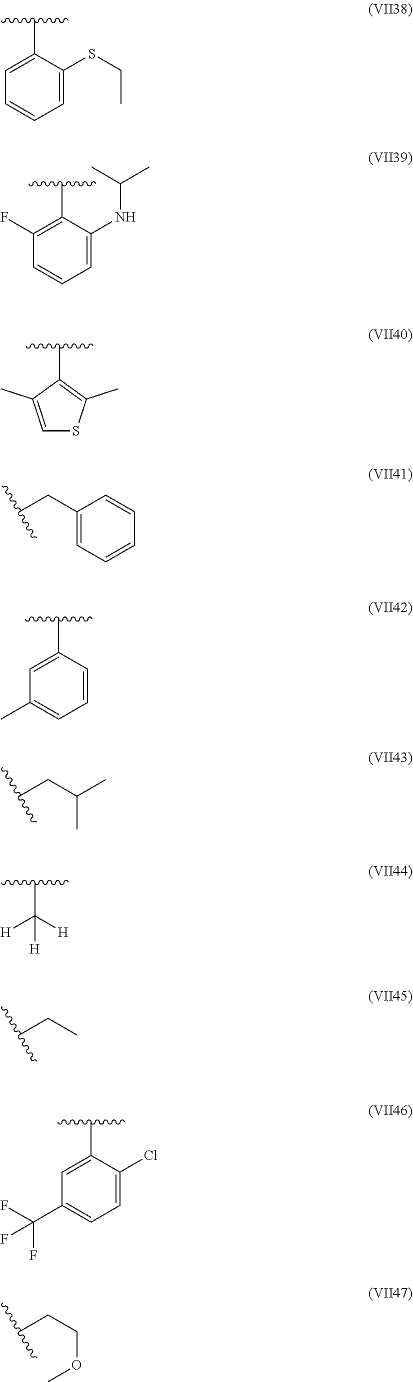

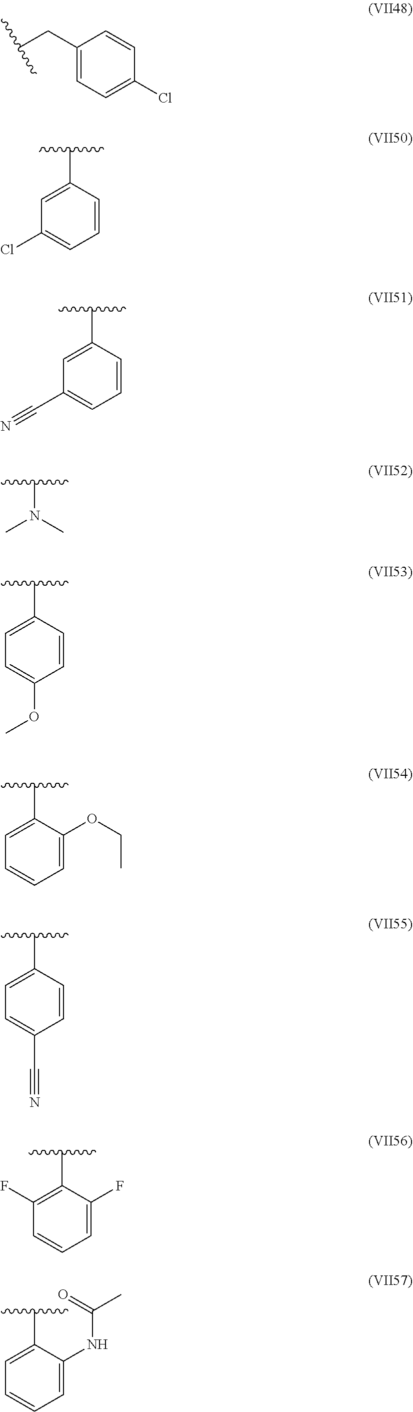

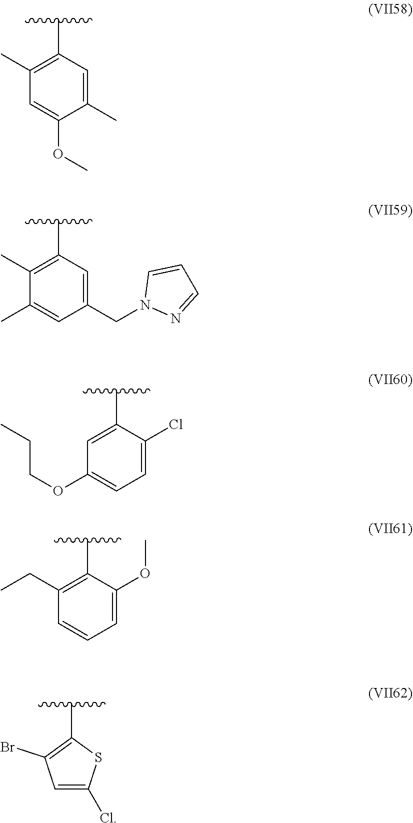

According to a further preferred embodiment of the invention, the use of a compound of the formula (I) for protecting the propagation material of plants is provided.

The invention also provides a composition comprising at least one compound of the formula (I) and customary extenders and/or surfactants, in particular for controlling animal pests.

The invention furthermore provides a method for controlling animal pests, in which at least one compound of the formula (I) or a composition according to the invention is allowed to act on the animal pests and/or their habitat.

According to a preferred embodiment of the method, the surgical, therapeutic and diagnostic treatment of the human or animal body is excluded.

The invention still furthermore provides an agrochemical formulation comprising at least one compound of the formula (I) according to any of Claims 1 to 8 in biologically effective amounts of from 0.00000001 to 98% by weight, based on the weight of the agrochemical formulation, and also extenders and/or surfactants.

A preferred embodiment of the formulation according to the invention additionally comprises a further agrochemically active compound.

The invention likewise provides compounds of the formula (VIII)

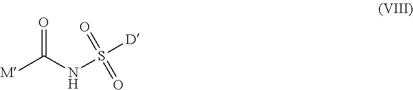

in which
M′ represents a radical of the formula (II) selected from:

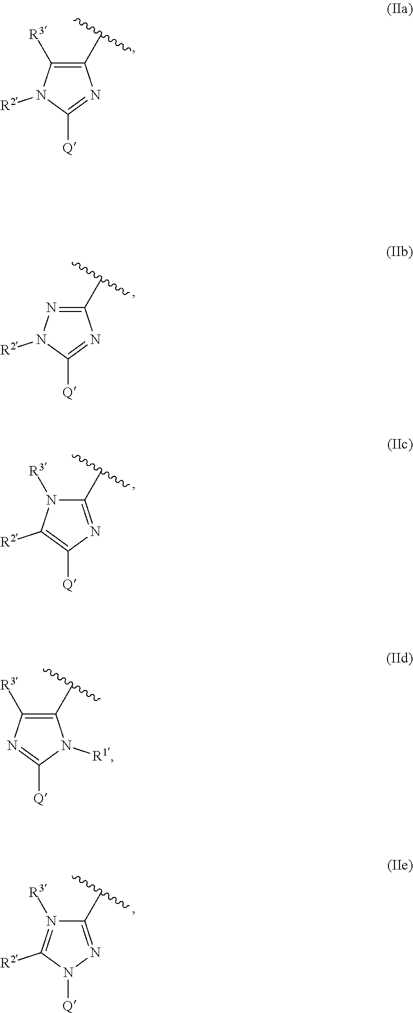

in which
R¹, R^{2′}, R^{3′} each independently of one another represent H or a substituted or unsubstituted alkyl, cycloalkyl, alkenyl, cycloalkenyl, cycloheteroalkyl, aryl, heteroaryl radical, where in cases (IIc) and (IIe) R² may additionally represent a halogen radical, in cases (IIa) and (IIb) R^{2′} may only represent H or a substituted or unsubstituted alkyl, cycloalkyl radical and in cases (IIa), (IId) and (IIe) R^{3′} may additionally represent a halogen radical;
Q′ represents a substituted or unsubstituted aryl or heteroaryl radical, but in cases (IIa), (IId) in case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl, nor 3,5-bis-tert-butyl;
D′ represents a substituted or unsubstituted alkyl, heteroalkyl, optionally partially unsaturated cycloalkyl, cycloheteroalkyl, heteroaryl, aryl or phenylalkyl radical or in the case that Q′ carries at least one substituent in the 2-position represents a substituted or unsubstituted nitrogen radical.

According to a first preferred embodiment of the compounds according to the invention,

Q′ represents a substituted or unsubstituted phenyl, naphthyl, heteroaryl radical which may contain one to three heteroatoms from the group consisting of oxygen, sulphur, nitrogen;
D′ represents a substituted or unsubstituted alkyl, heteroalkyl, cycloalkyl, heteroaryl, aryl or phenyl(C₁-C₈)-alkyl radical or, in the case that Q′ carries at least one substituent in the 2-position, represents a substituted or unsubstituted nitrogen radical;
in case (IIa) R^{2′}, R^{3′} each independently of one another represent H or a substituted or unsubstituted C₁-C₄-alkyl, C₃-C₆-cycloalkyl radical, where R^{3′} may additionally represent a halogen radical,
in case (IIb) R^{2′} represents H or a substituted or unsubstituted C₁-C₄-alkyl or C₃-C₆-cycloalkyl radical,
in case (IIc) R^{2′}, R^{3′} each independently of one another represent H or a substituted or unsubstituted C₁-C₄-alkyl, C₃-C₆-cycloalkyl or phenyl radical, in case (IId) R^{1′}, R^{3′} each independently of one another represent H or a substituted or unsubstituted C₁-C₄-alkyl, C₃-C₆-cycloalkyl radical and R^{3′} may additionally represent a halogen radical;
in case (IIe) R^{2′}, R^{3′} each independently of one another represent H or halogen or a substituted or unsubstituted C₁-C₄-alkyl, C₃-C₆-cycloalkyl radical.

More preferably

Q′ represents a phenyl, naphthyl, pyridyl, pyrimidinyl, thiophene or pyrazole radical; where the substituent(s) R^{4′} each independently of one another represent:
hydrogen, cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino; and/or
aryl or hetaryl, optionally mono- or polysubstituted by identical or different substituents, where (in the case of hetaryl) optionally at least one carbonyl group may be present and/or where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino;
D′ represents a radical, unsubstituted or substituted by one or more radicals R^{5′}, from the group consisting of C₁-C₆-alkyl, phenyl and heteroaryl which may contain one to three heteroatoms from the group consisting of oxygen, sulphur, nitrogen, or, in the case that Q′ carries at least one substituent in the 2-position, represents an NR^{6′}R^{7′} radical,
where the substituent(s) R^{5′} each independently of one another represent(s):
hydrogen, cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, (1-pyrazolyl)-(C₁-C₃)-alkyl,
and/or
aryl or hetaryl, mono- or polysubstituted by identical or different substituents, where (in the case of hetaryl) optionally at least one carbonyl group may be present and/or where possible substituents in each case are as follows: cyano, carboxyl, halogen, nitro, acetyl, hydroxy, amino, SCN, tri-(C₁-C₆)alkylsilyl, (C₃-C₈)cycloalkyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkyl, (C₁-C₆)haloalkyl, (C₁-C₆)cyanoalkyl, (C₁-C₆)hydroxyalkyl, hydroxycarbonyl-(C₁-C₆)-alkoxy, (C₁-C₆)alkoxycarbonyl-(C₁-C₆)alkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkoxycarbonyl-(C₁-C₆)alkoxy, (C₁-C₆)alkoxy-(C₁-C₆)alkoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylthio, (C₁-C₆)haloalkylthio, (C₁-C₆)alkoxy-(C₁-C₆)alkylthio, (C₁-C₆)alkylthio-(C₁-C₆)alkyl, (C₁-C₆)alkylsulphinyl, (C₁-C₆)haloalkylsulphinyl, (C₁-C₆)alkoxy-(C₁-C₆)alkylsulphinyl, (C₁-C₆)alkylsulphinyl-(C₁-C₆)alkyl, (C₁-C₆)alkylsulphonyl, (C₁-C₆)haloalkylsulphonyl, (C₁-C₆)alkoxy-(C₁-C₆)alkylsulphonyl, (C₁-C₆)alkylsulphonyl-(C₁-C₆)alkyl, (C₁-C₆)alkylsulphonyloxy, (C₁-C₆)alkylcarbonyl, (C₁-C₆)haloalkylcarbonyl, (C₁-C₆)alkylcarbonyloxy, (C₁-C₆)alkoxycarbonyl, (C₁-C₆)haloalkoxycarbonyl, aminocarbonyl, (C₁-C₆)alkylaminocarbonyl, di-(C₁-C₆)alkylaminocarbonyl, (C₂-C₆)alkenylaminocarbonyl, di-(C₂-C₆)-alkenylaminocarbonyl, (C₃-C₈)cycloalkylaminocarbonyl, (C₁-C₆)alkylsulphonylamino, (C₁-C₆)alkylamino, di-(C₁-C₆)alkylamino, aminosulphonyl, (C₁-C₆)alkylaminosulphonyl, di-(C₁-C₆)alkylaminosulphonyl, (C₁-C₆)alkylsulphoximino, aminothiocarbonyl, (C₁-C₆)alkylaminothiocarbonyl, di-(C₁-C₆)alkylaminothiocarbonyl, (C₃-C₈)cycloalkylamino, (C₁-C₆)alkylcarbonylamino,
and where R^{6′} and R^{7′} each independently of one another represent H, C₁-C₆-alkyl or a substituted or an unsubstituted phenyl radical or R^{6′} and R^{7′} together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R^{5′}.

Even more preferably

the substituent(s) R^{4′} each independently of one another represent(s):
hydrogen, cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino;
D′ represents a phenyl, pyridine, pyrimidine, pyrazole, triazole, thiazole, oxazole, thiadiazole, oxadiazole, thiophene, pyrrole, furan, tetrahydrofuran, dioxane or C₁-C₆-alkyl radical which is unsubstituted or substituted by one or more radicals R^{5′}, or represents an NR^{6′}R^{7′} radical,
where the substituent(s) R^{5′} each independently of one another represent(s):
hydrogen, cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy(C₁-C₆)-alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulphonylamino, (C₁-C₆)-alkylamino, di-(C₁-C₆)-alkylamino, aminosulphonyl, (C₁-C₆)alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino, (1-pyrazolyl)-(C₁-C₃)-alkyl,
and where R^{6′} and R^{7′} each independently of one another represent H, a C₁-C₆-alkyl, (C₁-C₆)haloalkyl, (C₁-C₆)-alkoxy-(C₁-C₆)-alkyl radical or an unsubstituted phenyl radical or a phenyl radical substituted by halogen, C₁-C₆-alkyl, (C₁-C₆)-haloalkyl, (C₁-C₆)-alkoxy-(C₁-C₆)-alkyl.

Special preference is given to an embodiment where

M′ represents a radical selected from the radicals of the formulae (IIa), (IIb), (IIc) (IId), (IIe)

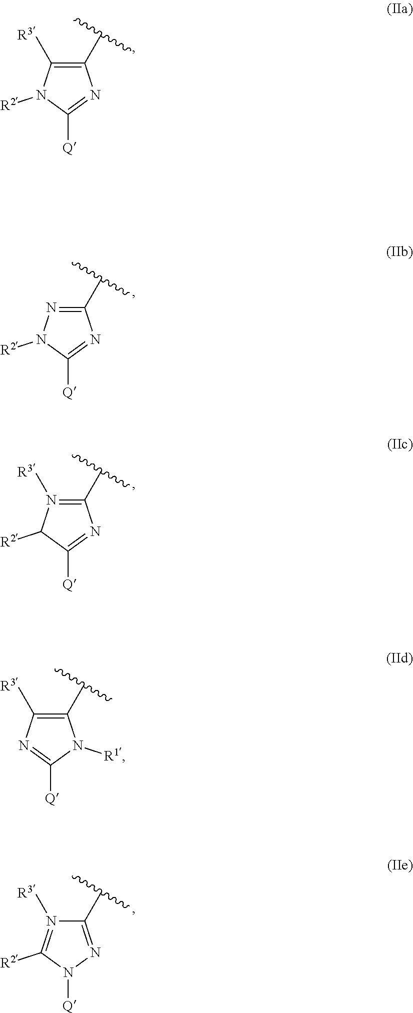

where
in case (IIa) R^{2′}, R^{3′} each independently of one another represent H or a substituted or unsubstituted C₁-C₄-alkyl or C₃-C₆-cycloalkyl radical, where R^{3′} may additionally represent a halogen radical,
in case (IIb) R^{2′} represents H or a substituted or unsubstituted C₁-C₄-alkyl or C₃-C₆-cycloalkyl radical,
in case (IIc) R^{2′}, R^{3′} each independently of one another represent H or a substituted or unsubstituted C₁-C₄-alkyl, C₃-C₆-cycloalkyl or phenyl radical,
in case (IId) R^{2′}, R^{3′} each independently of one another represent H or a substituted or unsubstituted C₁-C₄-alkyl, C₃-C₆-cycloalkyl radical and R^{3′} may additionally represent a halogen radical,
in case (IIe) R^{2′}, R^{3′} each independently of one another represent H or halogen or a substituted or unsubstituted C₁-C₄-alkyl, C₃-C₆-cycloalkyl radical;
D′ represents a C₁-C₆-alkyl radical or phenyl radical or pyridine, pyrimidine, pyrazole, triazole, thiazole, oxazole, thiadiazole, oxadiazole, thiophene, pyrrole, furan, tetrahydrofuran, dioxane radical which is unsubstituted or substituted by one or more radicals R^{5′}, or represents an NR^{6′}R^{7′} radical, where
R^{6′} and R^{7′} each independently of one another represent H, a C₁-C₆-alkyl, (C₁-C₆)-haloalkyl, (C₁-C₆)alkoxy-(C₁-C₆)-alkyl radical or unsubstituted phenyl or halogen-, C₁-C₆-alkyl-, (C₁-C₆)-haloalkyl-, (C₁-C₆)-alkoxy-(C₁-C₆)-alkyl-substituted phenyl radical
or form a ring from the group pyrrolidine, morpholine, piperidine.

Very particularly preferably,

M′ represents a radical selected from the radicals of the formulae (IVa), (IVb), (IVc) (IVd), (IVe),

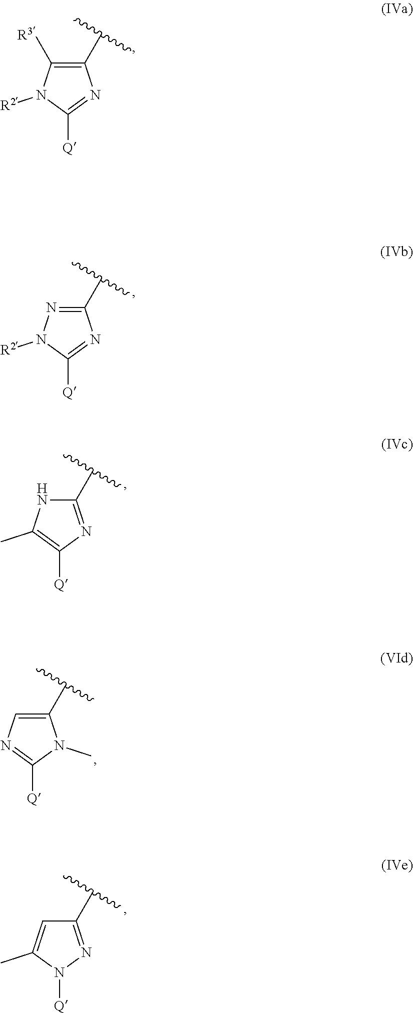

where
in case (IVa) R^{2′} represents H, methyl or ethyl, R^{3′} represents H, methyl, ethyl or halogen;
in case (IVb) R^{2′} represents H, methyl or ethyl;
Q′ represents a phenyl, naphthyl, pyridyl, pyrimidinyl, thiophene or pyrazole radical which is unsubstituted or substituted by one or more radicals R^{4′};
D′ represents a phenyl, pyridine, pyrimidine, pyrazole, triazole, thiazole, oxazole, thiadiazole, oxadiazole, thiophene, pyrrole, furan, tetrahydrofuran, dioxane or C₁-C₆-alkyl radical which is unsubstituted or substituted by one or more radicals R^{5′}, or represents an NR^{6′}R^{7′} radical.

Even more preferably

Q′ represents a phenyl, naphthyl, pyridyl, pyrimidinyl, thiophene or pyrazole radical which is unsubstituted or substituted by one or more radicals R^{4′};
where the substituent(s) R^{4′} independently of one another represent(s):
hydrogen, cyano, halogen, nitro, acetyl, (C₃-C₆)-cycloalkyl, (C₃-C₆)-cycloalkyloxy, (C₃-C₆)cycloalkyl-(C₃-C₆)-cycloalkyl, (C₁-C₆)-alkyl-(C₃-C₈)-cycloalkyl, halo-(C₃-C₆)-cycloalkyl, (C₁-C₆)alkyl, (C₁-C₆)-haloalkyl, (C₁-C₆)-cyanoalkyl, (C₁-C₆)-hydroxyalkyl, (C₁-C₆)-alkoxycarbonyl-(C₁-C₆)alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkylsulphonyl, (C₁-C₆)haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino);
D′ represents a C₁-C₆-alkyl radical or phenyl radical or pyridine, pyrimidine, pyrazole, triazole, thiazole, oxazole, thiadiazole, oxadiazole, thiophene, pyrrole, furan, tetrahydrofuran, dioxane radical which is unsubstituted or substituted by one or more radicals R^{5′},
where the substituent(s) R^{5′} independently of one another represent(s):
hydrogen, cyano, halogen, nitro, acetyl, hydroxy, carboxy, amino, (C₃-C₆)-cycloalkyl, (C₃-C₆)cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)cycloalkyl, (C₁-C₆)-alkyl, (C₁-C₆)-haloalkyl, (C₁-C₆)-cyanoalkyl, (C₁-C₆)-hydroxyalkyl, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)-haloalkylsulphinyl, (C₁-C₆)alkylsulphonyl, (C₁-C₆)-haloalkylsulphonyl, (C₁-C₆)-alkylsulphonyloxy, (C₁-C₆)-alkylcarbonyl, (C₁-C₆)-alkylamino, di-(C₁-C₆)-alkylamino, (C₁-C₆)-alkylcarbonylamino, (1-pyrazolyl)-(C₁-C₃)-alkyl,
R^{6′} and R^{7′} each independently of one another
represent H, a C₁-C₆-alkyl, (C₁-C₆)-haloalkyl, (C₁-C₆)-alkoxy-(C₁-C₆)-alkyl radical or unsubstituted phenyl or halogen-, C₁-C₆-alkyl-, (C₁-C₆)-haloalkyl-, (C₁-C₆)-alkoxy-(C₁-C₆)-alkyl-substituted phenyl radical.

Finally, also particularly preferably

M′ represents a radical selected from the radicals of the formulae (Va-Vr):

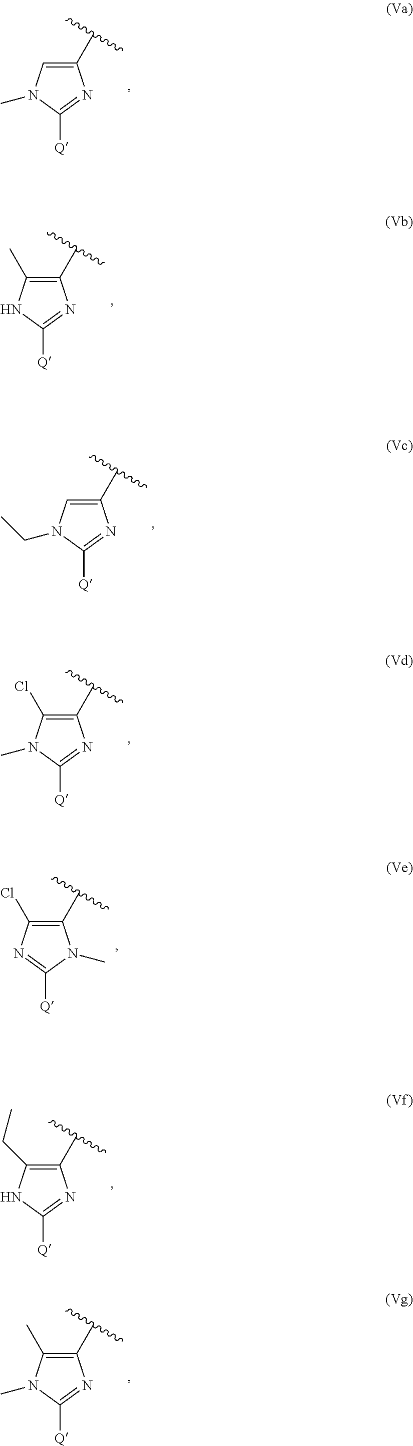

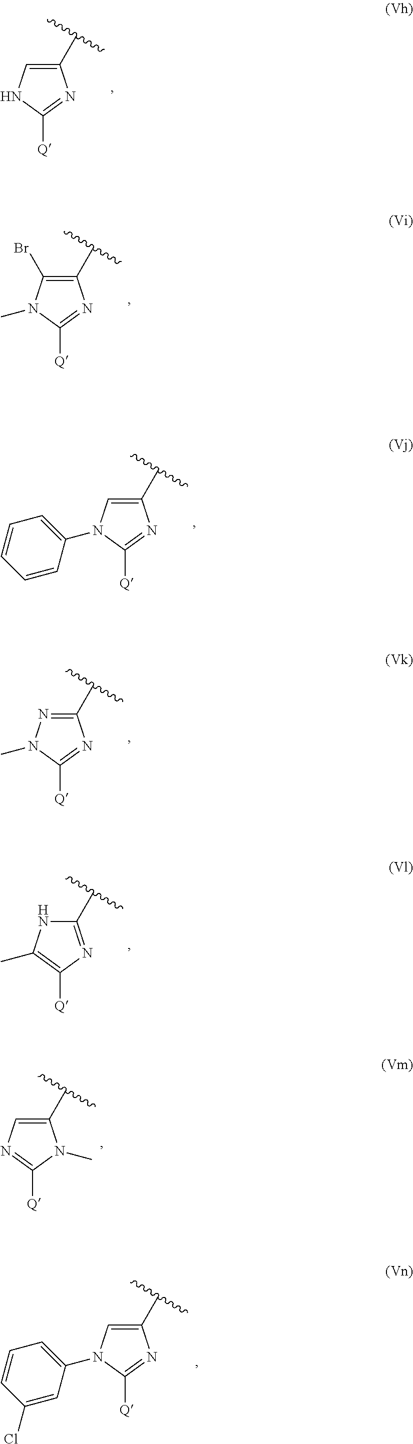

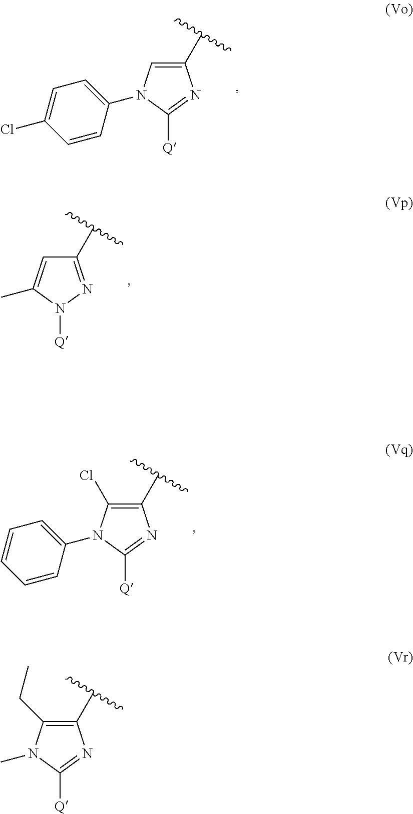

Q′ represents a radical selected from the radicals of the formulae (VIa-VIv):

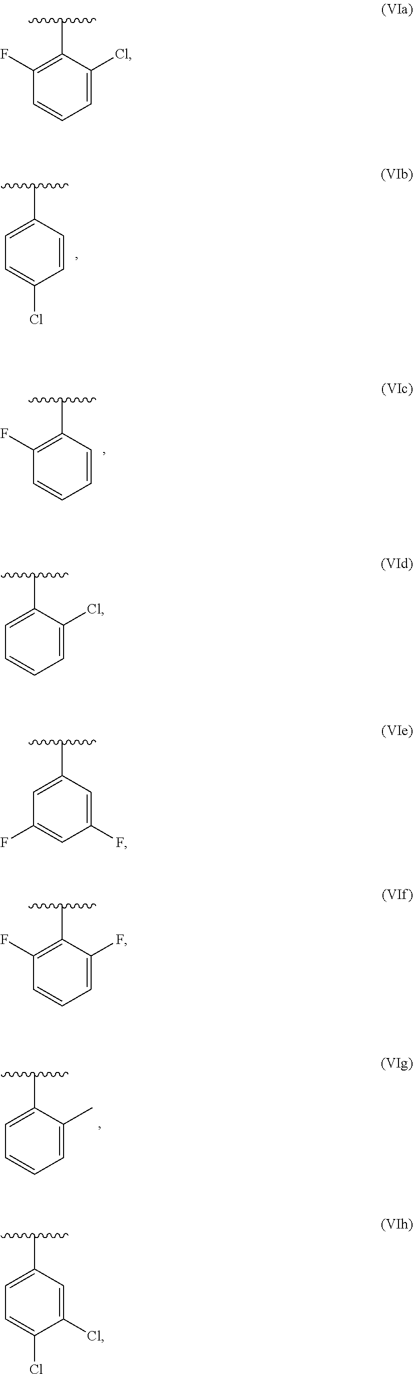

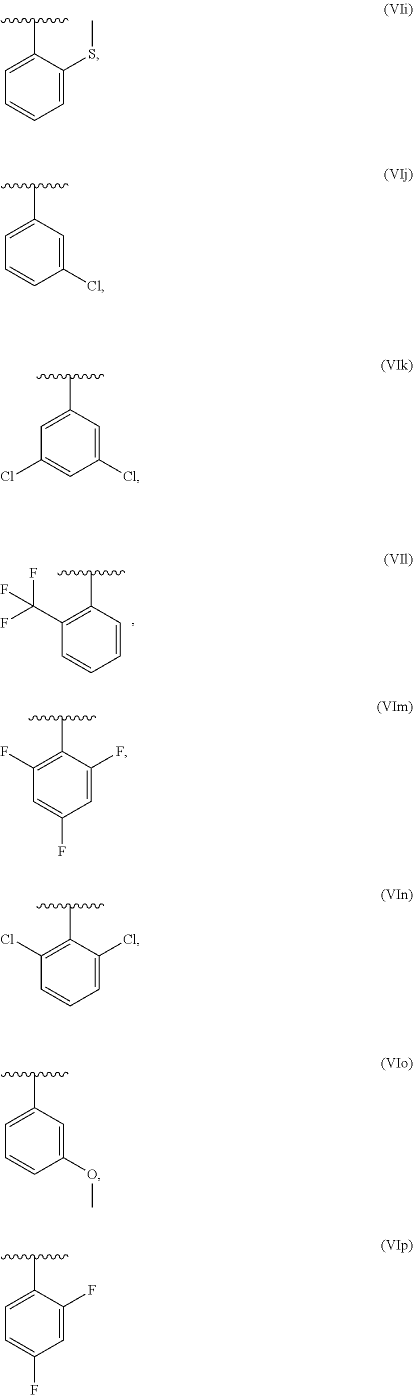

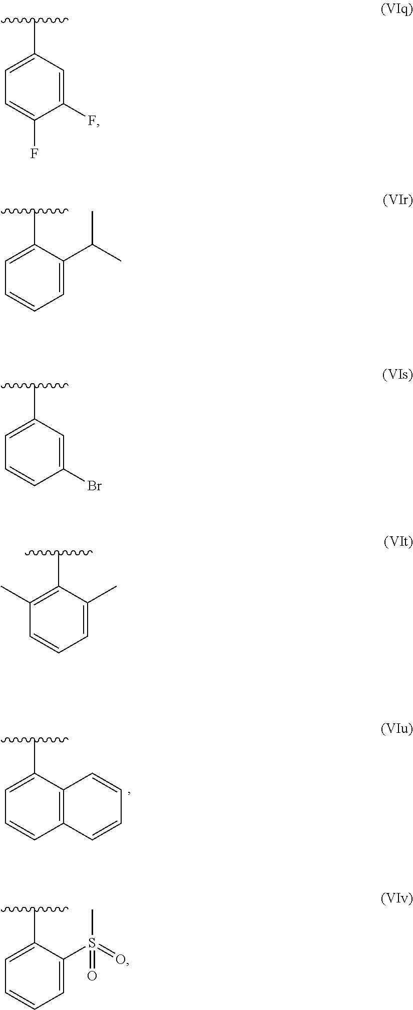

D′ represents a radical selected from the radicals of the formulae (VII1-VII62)

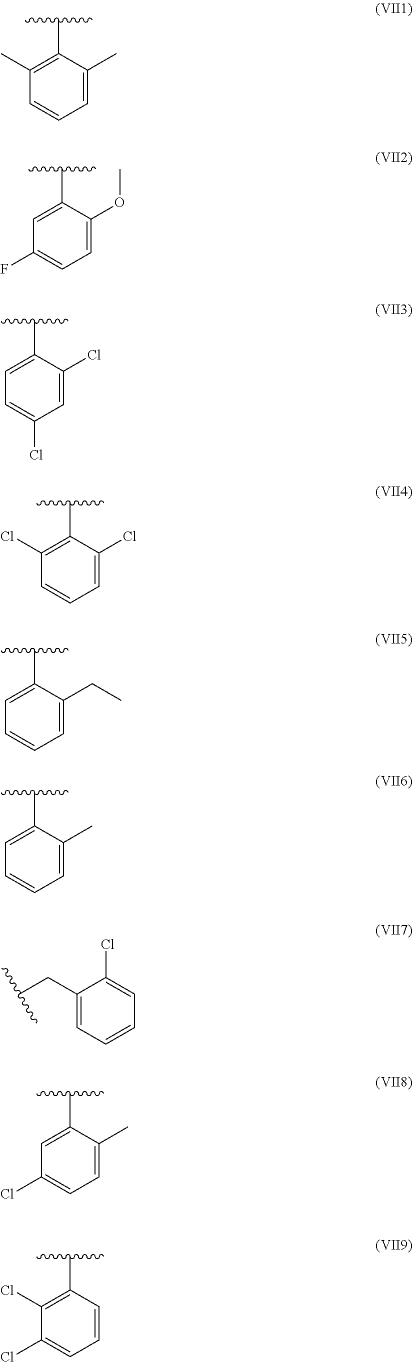

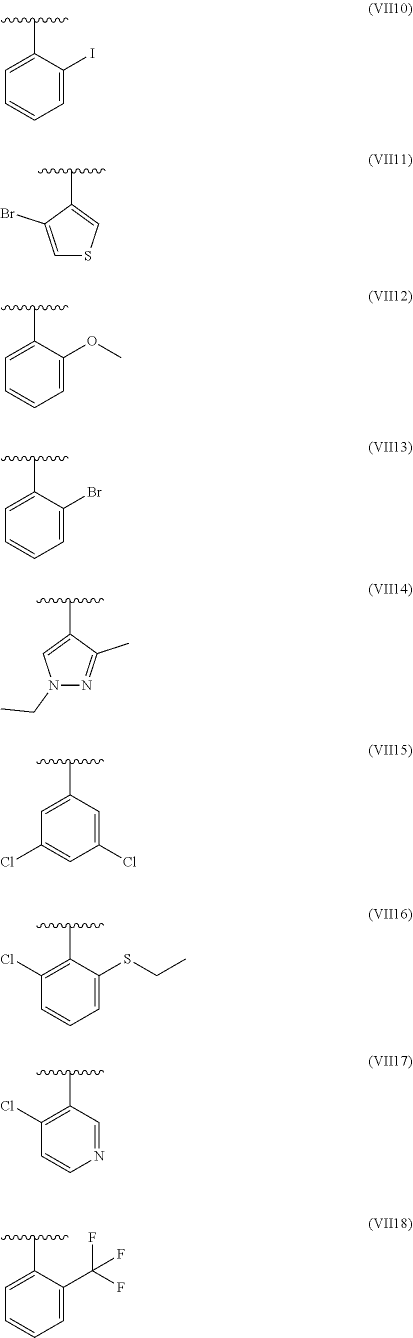

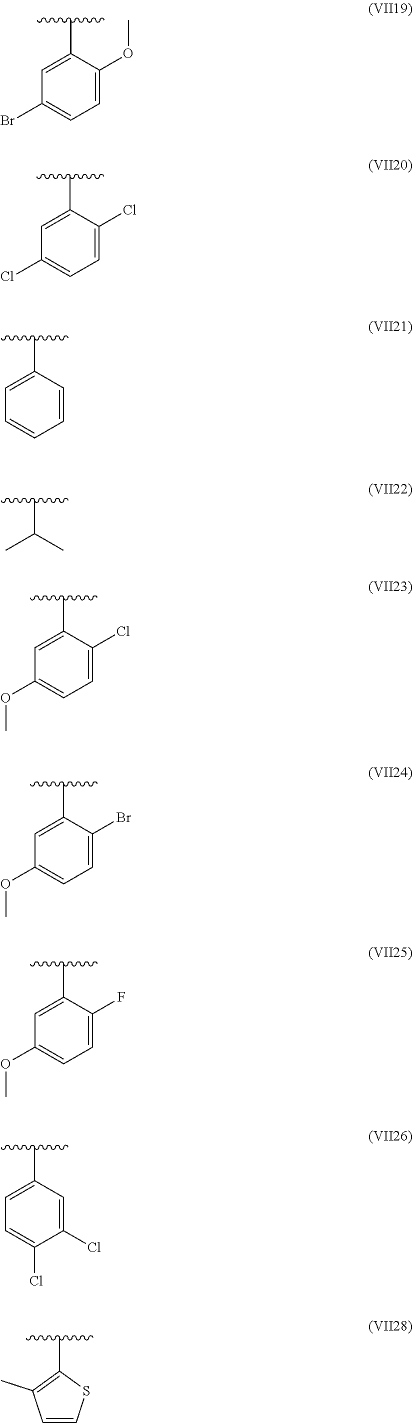

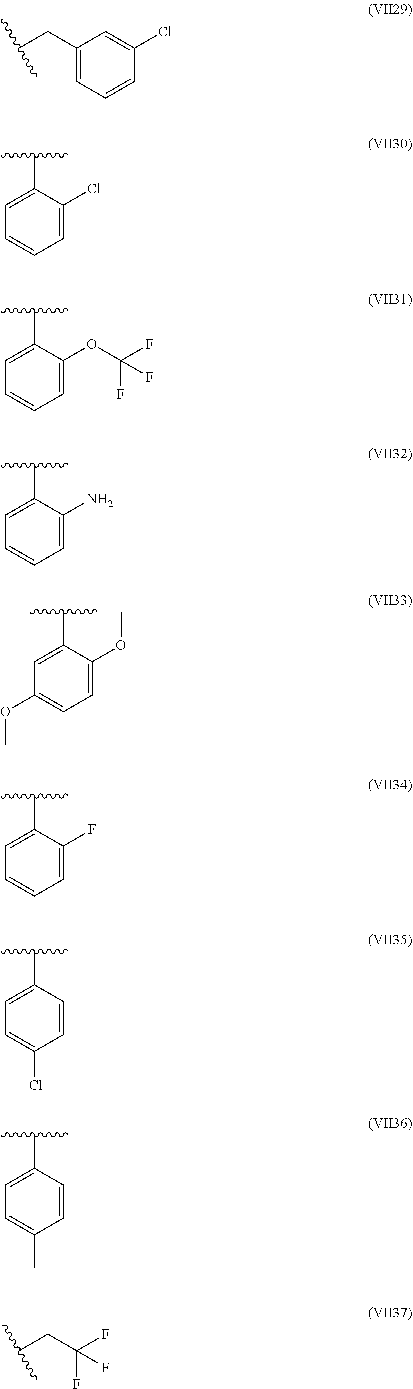

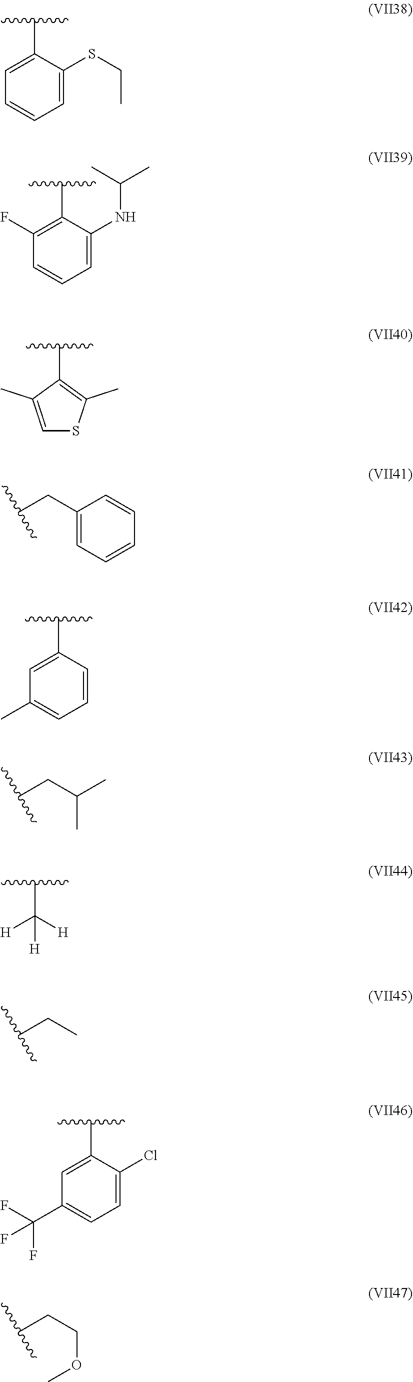

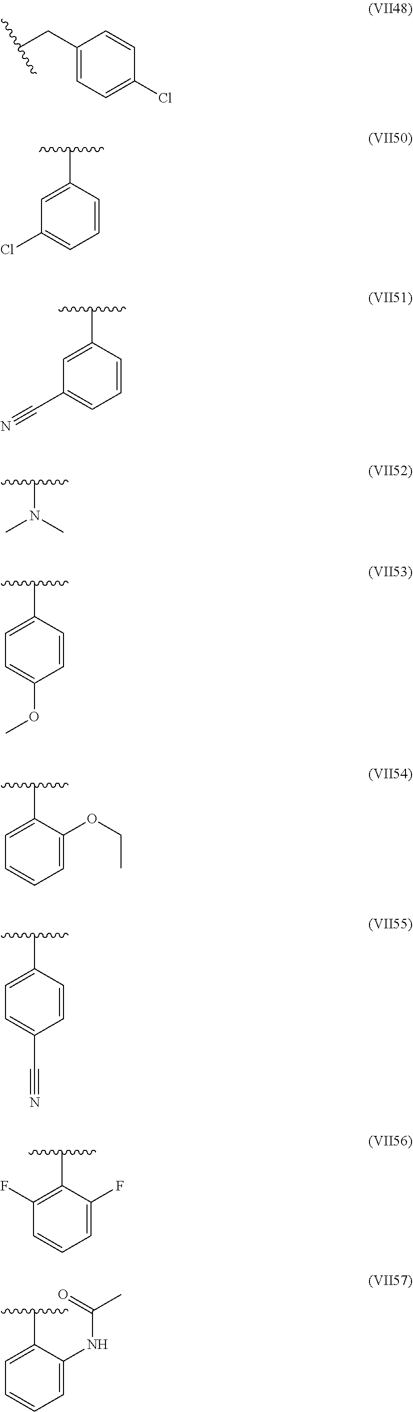

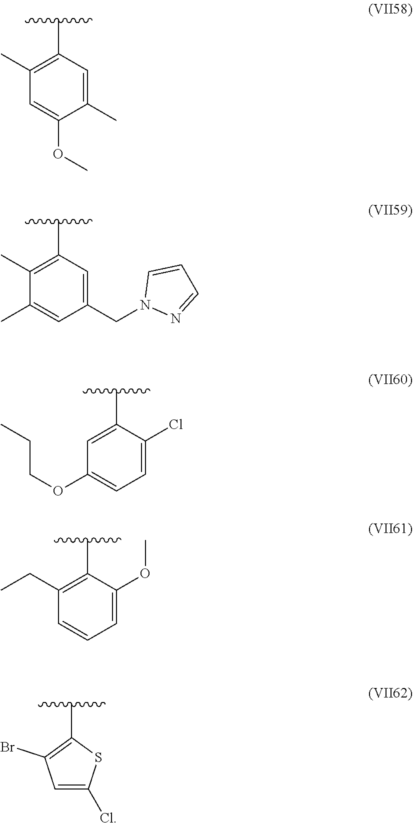

Hereinbelow, the expression formula (I) has the same meaning as formula (I) or formula (VIII), i.e. all statements analogously also apply to compounds of the formula (VIII).

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention therefore encompasses both pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, in particular crop protection agents.

In the context of the present application, the term “pesticide” in each case also always encompasses the term “crop protection agent”.

The compounds of the formula (I), given good plant tolerance, favourable endotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, especially nematodes and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” should be understood to mean any and all measures, provisions and procedures which have the aim of preventing diseases, especially infection diseases, and which serve to protect the health of humans and animals and/or protect the environment and/or maintain cleanliness. According to the invention, this especially includes measures for cleaning, disinfection and sterilization, for example of textiles or hard surfaces, especially surfaces made of glass, wood, cement, porcelain, ceramic, plastic or else metal(s), in order to ensure that these are free of hygiene pests and/or their secretions. The scope of protection of the invention in this regard preferably excludes surgical or therapeutic treatment procedures to be applied to the human body or the bodies of animals, and diagnostic procedures which are conducted on the human body or the bodies of animals.

The term “hygiene sector” covers all areas, technical fields and industrial applications in which these hygiene measures, provisions and procedures are important, for example with regard to hygiene in kitchens, bakeries, airports, bathrooms, swimming pools, department stores, hotels, hospitals, stables, animal keeping, etc.

The term “hygiene pest” should therefore be understood to mean one or more animal pests whose presence in the hygiene sector is problematic, especially for reasons of health. A main aim is therefore that of avoiding, or limiting to a minimum degree, the presence of hygiene pests and/or the exposure to these in the hygiene sector. This can especially be achieved through the use of a pesticide which can be used both for prevention of infestation and for prevention of an existing infestation. It is also possible to use formulations which prevent or reduce exposure to pests. Hygiene pests include, for example, the organisms mentioned below.

The term “hygiene protection” thus covers all acts by which these hygiene measures, provisions and procedures are maintained and/or improved.

The compounds of the formula (I) can preferably be used as pesticides. They are active against normally sensitive and resistant species and also against all or specific stages of development. The abovementioned pests include:

pests from the phylum of the Arthropoda, in particular from the class of the Arachnida, for example Acarus spp., e.g. Acarus siro, Aceria kuko, Aceria sheldoni, Aculops spp., Aculus spp., e.g. Aculus fockeui, Aculus schlechtendali, Amblyomma spp., Amphitetranychus viennensis, Argas spp., Boophilus spp., Brevipalpus spp., e.g. Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., e.g. Eotetranychus hicoriae, Epitrimerus pyri, Eutetranychus spp., e.g. Eutetranychus banksi, Eriophyes spp., e.g. Eriophyes pyri, Glycyphagus domesticus, Halotydeus destructor, Hemitarsonemus spp., e.g. Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., e.g. Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., e.g. Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., e.g. Tarsonemus confusus, Tarsonemus pallidus, Tetranychus spp., e.g. Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea e.g. Blatta orientalis, Blattella asahinai, Blattella germanica, Leucophaea maderae, Loboptera decipiens, Neostylopyga rhombifolia, Panchlora spp., Parcoblatta spp., Periplaneta spp., e.g. Periplaneta americana, Periplaneta australasiae, Pycnoscelus surinamensis, Supella longipalpa;
from the order of the Coleoptera, for example Acalymma vittatum, Acanthoscelides obtectus, Adoretus spp., Aethina tumida, Agelastica alni, Agriotes spp., e.g. Agriotes linneatus, Agriotes mancus, Alphitobius diaperinus, Amphimallon solstitialis, Anobium punctatum, Anoplophora spp., Anthonomus spp., e.g. Anthonomus grandis, Anthrenus spp., Apion spp., Apogonia spp., Atomaria spp., e.g. Atomaria linearis, Attagenus spp., Baris caerulescens, Bruchidius obtectus, Bruchus spp., e.g. Bruchus pisorum, Bruchus rufimanus, Cassida spp., Cerotoma trifurcata, Ceutorrhynchus spp., e.g. Ceutorrhynchus assimilis, Ceutorrhynchus quadridens, Ceutorrhynchus rapae, Chaetocnema spp., e.g. Chaetocnema confinis, Chaetocnema denticulata, Chaetocnema ectypa, Cleonus mendicus, Conoderus spp., Cosmopolites spp., e.g. Cosmopolites sordidus, Costelytra zealandica, Ctenicera spp., Curculio spp., e.g. Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., e.g. Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., e.g. Epilachna borealis, Epilachna varivestis, Epitrix spp., e.g. Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., e.g. Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., e.g. Leucoptera coffeella, Lissorhoptrus oryzophilus, Listronotus (=Hyperodes) spp., Lixus spp., Luperodes spp., Luperomorpha xanthodera, Lyctus spp., Megascelis spp., Melanotus spp., e.g. Melanotus longulus oregonensis, Meligethes aeneus, Melolontha spp., e.g. Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., e.g. Otiorhynchus cribricollis, Otiorhynchus ligustici, Otiorhynchus ovatus, Otiorhynchus rugosostriarus, Otiorhynchus sulcatus, Oulema spp., e.g. Oulema melanopus, Oulema oryzae, Oxycetonia jucunda, Phaedon cochleariae, Phyllophaga spp., Phyllophaga helleri, Phyllotreta spp., e.g. Phyllotreta armoraciae, Phyllotreta pusilla, Phyllotreta ramosa, Phyllotreta striolata, Popillia japonica, Premnotrypes spp., Prostephanus truncatus, Psylliodes spp., e.g. Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Sinoxylon perforans, Sitophilus spp., e.g. Sitophilus granarius, Sitophilus linearis, Sitophilus oryzae, Sitophilus zeamais, Sphenophorus spp., Stegobium paniceum, Sternechus spp., e.g. Sternechus paludatus, Symphyletes spp., Tanymecus spp., e.g. Tanymecus dilaticollis, Tanymecus indicus, Tanymecus palliatus, Tenebrio molitor, Tenebrioides mauretanicus, Tribolium spp., e.g. Tribolium audax, Tribolium castaneum, Tribolium confusum, Trogoderma spp., Tychius spp., Xylotrechus spp., Zabrus spp., e.g. Zabrus tenebrioides;
from the order of the Dermaptera, for example Anisolabis maritime, Forficula auricularia, Labidura riparia;
from the order of the Diptera, for example Aedes spp., e.g. Aedes aegypti, Aedes albopictus, Aedes sticticus, Aedes vexans, Agromyza spp., e.g. Agromyza frontella, Agromyza parvicornis, Anastrepha spp., Anopheles spp., e.g. Anopheles quadrimaculatus, Anopheles gambiae, Asphondylia spp., Bactrocera spp., e.g. Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., e.g. Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., e.g. Culex pipiens, Culex quinquefasciatus, Culicoides spp., Culiseta spp., Cuterebra spp., Dacus oleae, Dasineura spp., e.g. Dasineura brassicae, Delia spp., e.g. Delia antiqua, Delia coarctata, Delia florilega, Delia platura, Delia radicum, Dermatobia hominis, Drosophila spp., e.g. Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., e.g. Liriomyza brassicae, Liriomyza huidobrensis, Liriomyza sativae, Lucilia spp., e.g. Lucilia cuprina, Lutzomyia spp., Mansonia spp., Musca spp., e.g. Musca domestica, Musca domestica vicina, Oestrus spp., Oscinella frit, Paratanytarsus spp., Paralauterborniella subcincta, Pegomya oder Pegomyia spp., e.g., Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., e.g. Rhagoletis cingulata, Rhagoletis completa, Rhagoletis fausta, Rhagoletis indifferens, Rhagoletis mendax, Rhagoletis pomonella, Sarcophaga spp., Simulium spp., e.g. Simulium meridionale, Stomoxys spp., Tabanus spp., Tetanops spp., Tipula spp., e.g. Tipula paludosa, Tipula simplex, Toxotrypana curvicauda;
from the order of the Hemiptera, for example Acizzia acaciaebaileyanae, Acizzia dodonaeae, Acizzia uncatoides, Acrida turrita, Acyrthosipon spp., e.g. Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., e.g. Amrasca bigutulla, Amrasca devastans, Anuraphis cardui, Aonidiella spp., e.g. Aonidiella aurantii, Aonidiella citrina, Aonidiella inornata, Aphanostigma piri, Aphis spp., e.g. Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., e.g. Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., e.g. Cacopsylla pyricola, Calligypona marginata, Capulinia spp., Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., e.g. Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., e.g. Dysaphis apiifolia, Dysaphis plantaginea, Dysaphis tulipae, Dysmicoccus spp., Empoasca spp., e.g. Empoasca abrupta, Empoasca fabae, Empoasca maligna, Empoasca solana, Empoasca stevensi, Eriosoma spp., e.g. Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., e.g. Icerya purchasi, Idiocerus spp., Idioscopus spp., Laodelphax striatellus, Lecanium spp., e.g. Lecanium comi (=Parthenolecanium comi), Lepidosaphes spp., e.g. Lepidosaphes ulmi, Lipaphis erysimi, Lopholeucaspis japonica, Lycorma delicatula, Macrosiphum spp., e.g. Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., e.g. Myzus ascalonicus, Myzus cerasi, Myzus ligustri, Myzus ornatus, Myzus persicae, Myzus nicotianae, Nasonovia ribisnigri, Neomaskellia spp., Nephotettix spp., e.g. Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., e.g. Paratrioza cockerelli, Parlatoria spp., Pemphigus spp., e.g. Pemphigus bursarius, Pemphigus populivenae, Peregrinus maidis, Perkinsiella spp., Phenacoccus spp., e.g. Phenacoccus madeirensis, Phloeomyzus passerinii, Phorodon humuli, Phylloxera spp., e.g. Phylloxera devastatrix, Phylloxera notabilis, Pinnaspis aspidistrae, Planococcus spp., e.g. Planococcus citri, Prosopidopsylla flava, Protopulvinaria pyriformis, Pseudaulacaspis pentagona, Pseudococcus spp., e.g. Pseudococcus calceolariae, Pseudococcus comstocki, Pseudococcus longispinus, Pseudococcus maritimus, Pseudococcus vibumi, Psyllopsis spp., Psylla spp., e.g. Psylla buxi, Psylla mali, Psylla pyri, Pteromalus spp., Pulvinaria spp., Pyrilla spp., Quadraspidiotus spp., e.g. Quadraspidiotus juglansregiae, Quadraspidiotus ostreaeformis, Quadraspidiotus perniciosus, Quesada gigas, Rastrococcus spp., Rhopalosiphum spp., e.g. Rhopalosiphum maidis, Rhopalosiphum oxyacanthae, Rhopalosiphum padi, Rhopalosiphum rufiabdominale, Saissetia spp., e.g. Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., e.g. Toxoptera aurantii, Toxoptera citricidus, Trialeurodes vaporariorum, Trioza spp., e.g. Trioza diospyri, Typhlocyba spp., Unaspis spp., Viteus vitifolii, Zygina spp.;
from the suborder of the Heteroptera, for example Aelia spp., Anasa tristis, Antestiopsis spp., Boisea spp., Blissus spp., Calocoris spp., Campylomma livida, Cavelerius spp., Cimex spp., e.g. Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., e.g. Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., e.g. Lygocoris pabulinus, Lygus spp., e.g. Lygus elisus, Lygus hesperus, Lygus lineolaris, Macropes excavatus, Megacopta cribraria, Miridae, Monalonion atratum, Nezara spp., e.g. Nezara viridula, Nysius spp., Oebalus spp., Pentomidae, Piesma quadrata, Piezodorus spp., e.g. Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., e.g. Athalia rosae, Atta spp., Camponotus spp., Dolichovespula spp., Diprion spp., e.g. Diprion similis, Hoplocampa spp., e.g. Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., Solenopsis invicta, Tapinoma spp., Technomyrmex albipes, Urocerus spp., Vespa spp., e.g. Vespa crabro, Wasmannia auropunctata, Xeris spp.;
from the order of the Isopoda, for example Armadillidium vulgare, Oniscus asellus, Porcellio scaber;
from the order of the Isoptera, for example Coptotermes spp., e.g. Coptotermes formosanus, Cornitermes cumulans, Cryptotermes spp., Incisitermes spp., Kalotermes spp., Microtermes obesi, Nasutitermis spp., Odontotermes spp., Porotermes spp., Reticulitermes spp., e.g. Reticulitermes flavipes, Reticulitermes hesperus;
from the order of the Lepidoptera, for example Achroia grisella, Acronicta major, Adoxophyes spp., e.g. Adoxophyes orana, Aedia leucomelas, Agrotis spp., e.g. Agrotis segetum, Agrotis ipsilon, Alabama spp., e.g. Alabama argillacea, Amyelois transitella, Anarsia spp., Anticarsia spp., e.g. Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., e.g. Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., e.g. Cydia nigricana, Cydia pomonella, Dalaca noctuides, Diaphania spp., Diparopsis spp., Diatraea saccharalis, Earias spp., Ecdytolopha aurantium, Elasmopalpus lignosellus, Eldana saccharina, Ephestia spp., e.g. Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., e.g. Euproctis chrysorrhoea, Euxoa spp., Feltia spp., Galleria mellonella, Gracillaria spp., Grapholitha spp., e.g. Grapholita molesta, Grapholita prunivora, Hedylepta spp., Helicoverpa spp., e.g. Helicoverpa armigera, Helicoverpa zea, Heliothis spp., e.g. Heliothis virescens Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., e.g. Leucoptera coffeella, Lithocolletis spp., e.g. Lithocolletis blancardella, Lithophane antennata, Lobesia spp., e.g. Lobesia botrana, Loxagrotis albicosta, Lymantria spp., e.g. Lymantria dispar, Lyonetia spp., e.g. Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., e.g. Ostrinia nubilalis, Panolis flammea, Parnara spp., Pectinophora spp., e.g. Pectinophora gossypiella, Perileucoptera spp., Phthorimaea spp., e.g. Phthorimaea operculella, Phyllocnistis citrella, Phyllonorycter spp., e.g. Phyllonorycter blancardella, Phyllonorycter crataegella, Pieris spp., e.g. Pieris rapae, Platynota stultana, Plodia interpunctella, Plusia spp., Plutella xylostella (=Plutella maculipennis), Prays spp., Prodenia spp., Protoparce spp., Pseudaletia spp., e.g. Pseudaletia unipuncta, Pseudoplusia includens, Pyrausta nubilalis, Rachiplusia nu, Schoenobius spp., e.g. Schoenobius bipunctifer, Scirpophaga spp., e.g. Scirpophaga innotata, Scotia segetum, Sesamia spp., e.g. Sesamia inferens, Sparganothis spp., Spodoptera spp., e.g. Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., e.g. Trichoplusia ni, Tryporyza incertulas, Tuta absoluta, Virachola spp.;
from the order of the Orthoptera or Saltatoria, for example Acheta domesticus, Dichroplus spp., Gryllotalpa spp., e.g. Gryllotalpa gryllotalpa, Hieroglyphus spp., Locusta spp., e.g. Locusta migratoria, Melanoplus spp., e.g. Melanoplus devastator, Paratlanticus ussuriensis, Schistocerca gregaria;
from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example Ceratophyllus spp., Ctenocephalides spp., e.g. Ctenocephalides canis, Ctenocephalides felis, Pulex irritans, Tunga penetrans, Xenopsylla cheopis;
from the order of the Thysanoptera, e.g. Anaphothrips obscurus, Baliothrips biformis, Chaetanaphothrips leeuweni, Drepanothrips reuteri, Enneothrips flavens, Frankliniella spp., e.g. Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., e.g. Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., e.g. Scutigerella immaculata;
pests from the phylum of the Mollusca, for example from the class of the Bivalvia, e.g. Dreissena spp.;
from the class of the Gastropoda, for example Arion spp., e.g. Arion ater rufus, Biomphalaria spp., Bulinus spp., Deroceras spp., e.g. Deroceras laeve, Galba spp., Lymnaea spp., Oncomelania spp., Pomacea spp., Succinea spp.;
plant pests from the phylum of the Nematoda, i.e. plant-parasitic nematodes, in particular Aglenchus spp., e.g. Aglenchus agricola, Anguina spp., e.g. Anguina tritici, Aphelenchoides spp., e.g. Aphelenchoides arachidis, Aphelenchoides fragariae, Belonolaimus spp., e.g. Belonolaimus gracilis, Belonolaimus longicaudatus, Belonolaimus nortoni, Bursaphelenchus spp., e.g. Bursaphelenchus cocophilus, Bursaphelenchus eremus, Bursaphelenchus xylophilus, Cacopaurus spp., e.g. Cacopaurus pestis, Criconemella spp., e.g. Criconemella curvata, Criconemella onoensis, Criconemella ornata, Criconemella rusium, Criconemella xenoplax (=Mesocriconema xenoplax), Criconemoides spp., e.g. Criconemoides ferniae, Criconemoides onoense, Criconemoides ornatum, Ditylenchus spp., e.g. Ditylenchus dipsaci, Dolichodorus spp., Globodera spp., e.g. Globodera pallida, Globodera rostochiensis, Helicotylenchus spp., e.g. Helicotylenchus dihystera, Hemicriconemoides spp., Hemicycliophora spp., Heterodera spp., e.g. Heterodera avenae, Heterodera glycines, Heterodera schachtii, Hirschmaniella spp., Hoplolaimus spp., Longidorus spp., e.g. Longidorus africanus, Meloidogyne spp., e.g. Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., e.g. Paratrichodorus minor, Paratylenchus spp., Pratylenchus spp., e.g. Pratylenchus penetrans, Pseudohalenchus spp., Psilenchus spp., Punctodera spp., Quinisulcius spp., Radopholus spp., e.g. Radopholus citrophilus, Radopholus similis, Rotylenchulus spp., Rotylenchus spp., Scutellonema spp., Subanguina spp., Trichodorus spp., e.g. Trichodorus obtusus, Trichodorus primitivus, Tylenchorhynchus spp., e.g. Tylenchorhynchus annulatus, Tylenchulus spp., e.g. Tylenchulus semipenetrans, Xiphinema spp., e.g. Xiphinema index.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active compounds.

### Formulations

The present invention further relates to formulations and use forms prepared therefrom as pesticides, for example drench, drip and spray liquors, comprising at least one compound of the formula (I). Optionally, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemically active compounds.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compounds of the formula (I) with auxiliaries, for example extenders, solvents and/or solid carriers and/or other auxiliaries, for example surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed-dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as Nalkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and their ethers and esters, ketones, for example acetone, methyl ethyl ketone, methyl isobutyl ketone or cyclohexanone, strongly polar solvents, for example dimethyl sulphoxide, and water.

In principle, it is possible to use all suitable carriers. Useful carriers especially include, for example, ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable extenders or carriers are those which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and if the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulphosuccinate, or increase viscoelasticity, for example hydroxypropylguar polymers.

Useful penetrants in the present context are all those substances which are typically used to improve the penetration of active agrochemical ingredients into plants. Penetrants are defined in this context by their ability to penetrate from the (generally aqueous) application liquor and/or from the spray coating into the cuticle of the plant and hence to increase the mobility of the active ingredients in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active compounds or semiochemicals such as attractants and/or bird repellents and/or plant activators and/or growth regulators and/or fertilizers. Likewise, the compounds of the formula (I) can be used to improve plant properties, for example growth, yield and quality of the harvested material.

In a particular embodiment according to the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides/Acaricides/Nematicides

The active compounds specified here with their common names are known and are described for example in “The Pesticide Manual”, 16th ed., British Crop Protection Council 2012, or can be searched for on the Internet (e.g. http://www.alanwood.net/pesticides). The classification is based on the IRAC Mode of Action Classification Scheme applicable at the time of filing of this patent application.

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphosmethyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel blockers, for example cyclodiene-organochlorines, e.g. chlordane and endosulfan or phenylpyrazoles (fiproles), e.g. ethiprole and fipronil.

(3) Sodium channel modulators, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.

(4) Nicotinic acetylcholine receptor (nAChR) competitive modulators, for example neonicotinoids, e.g. acetamiprid, clothianidin, dinotefuran, imidacloprid, nitenpyram, thiacloprid and thiamethoxam or nicotine or sulfoxaflor or flupyradifurone.

(5) Nicotinic acetylcholine receptor (nAChR) allosteric modulators, for example spinosyns, e.g. spinetoram and spinosad.

(6) Glutamate-gated chloride channel (GluCl) allosteric modulators, for example avermectins/milbemycins, e.g. abamectin, emamectin benzoate, lepimectin and milbemectin.

(7) Juvenile hormone mimetics, for example juvenile hormone analogues, e.g. hydroprene, kinoprene and methoprene or fenoxycarb or pyriproxyfen.

(8) Miscellaneous non-specific (multisite) inhibitors, for example alkyl halides, e.g. methyl bromide and other alkyl halides; or chloropicrin or sulphuryl fluoride or borax or tartar emetic or methyl isocyanate generator, e.g. diazomet and metam.

(9) Chordotonal organ modulators, e.g. pymetrozine or flonicamide.

(10) Mite growth inhibitors, for example clofentezine, hexythiazox and diflovidazin or etoxazole.

(11) Microbial disruptors of the insect midgut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, VIP3A, mCry3A, Cry3Ab, Cry3Bb, Cry34Ab1/35Ab1.

(12) Inhibitors of mitochondrial ATP synthase, such as ATP disruptors, for example diafenthiuron or organotin compounds, e.g. azocyclotin, cyhexatin and fenbutatin oxide or propargite or tetradifon.

(13) Uncouplers of oxidative phosphorylation via disruption of the proton gradient, for example chlorfenapyr, DNOC and sulfluramid.

(14) Nicotinic acetylcholine receptor channel blockers, for example bensultap, cartap hydrochloride, thiocyclam, and thiosultap-sodium.

(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, for example buprofezin.

(17) Moulting disruptors (especially in the case of Diptera), for example cyromazine.

(18) Ecdysone receptor agonists, for example chromafenozide, halofenozide, methoxyfenozide and tebufenozide.

(19) Octopamine receptor agonists, for example amitraz.

(20) Mitochondrial complex III electron transport inhibitors, for example hydramethylnon or acequinocyl or fluacrypyrim.

(21) Mitochondrial complex I electron transport inhibitors, for example METI acaricides, e.g. fenazaquin, fenpyroximate, pyrimidifen, pyridaben, tebufenpyrad and tolfenpyrad or rotenone (Derris).

(22) Voltage-dependent sodium channel blockers, for example indoxacarb or metaflumizone.

(23) Inhibitors of acetyl CoA carboxylase, for example tetronic and tetramic acid derivatives, e.g. spirodiclofen, spiromesifen and spirotetramat.

(24) Mitochondrial complex IV electron transport inhibitors, for example phosphines, e.g. aluminium phosphide, calcium phosphide, phosphine and zinc phosphide, or cyanides, calcium cyanide, potassium cyanide and sodium cyanide.

(25) Mitochondrial complex II electron transport inhibitors, for example beta-keto nitrile derivatives, e.g. cyenopyrafen and cyflumetofen and carboxanilides, for example pyflubumide.

(28) Ryanodine receptor modulators, for example diamides, e.g. chlorantraniliprole, cyantraniliprole and flubendiamide,

further active ingredients, for example afidopyropen, afoxolaner, azadirachtin, benclothiaz, benzoximate, bifenazate, broflanilide, bromopropylate, chinomethionat, chloroprallethrin, cryolite, cyclaniliprole, cycloxaprid, cyhalodiamide, dicloromezotiaz, dicofol, epsilon metofluthrin, epsilon momfluthrin, flometoquin, fluazaindolizine, fluensulfone, flufenerim, flufenoxystrobin, flufiprole, fluhexafon, fluopyram, fluralaner, fluxametamide, fufenozide, guadipyr, heptafluthrin, imidaclothiz, iprodione, kappa bifenthrin, kappa tefluthrin, lotilaner, meperfluthrin, paichongding, pyridalyl, pyrifluquinazon, pyriminostrobin, spirobudiclofen, tetramethylfluthrin, tetraniliprole, tetrachlorantraniliprole, tioxazafen, thiofluoximate, triflumezopyrim and iodomethane; additionally preparations based on Bacillus firmus (1-1582, BioNeem, Votivo), and the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indole-3,4′-piperidine]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS-1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS Reg. No. 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5 S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulphinyl]propanamide, (+)-N-[3-chloro-1-(3-pyridinyl)-1H-pyrazol-4-yl]-N-ethyl-3-[(3,3,3-trifluoropropyl)sulphinyl]propanamide and (−)-N-[3-chloro-1-(3-pyridinyl)-1H-pyrazol-4-yl]-N-ethyl-3-[(3,3,3-trifluoropropyl)sulphinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulphinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1Hpyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); cyclopropanecarboxylic acid 3-(2,2-dichloroethenyl)-2,2-dimethyl-4-(1H-benzimidazol-2-yl)phenyl ester (known from CN 103524422 A) (CAS 1542271-46-4); (4aS)-7-chloro-2,5-dihydro-2-[[(methoxycarbonyl) [4-[(trifluoromethyl)thio]phenyl]amino]carbony]indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-Omethyl-1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8) and N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9).

### Fungicides

The active compounds specified herein by their common name are known and described, for example, in “Pesticide Manual” (16th Ed. British Crop Protection Council) or searchable on the internet (for example: http://www.alanwood.net/pesticides).

All the mixing components mentioned in classes (1) to (15), as the case may be, may form salts with suitable bases or acids if they are capable of doing so on the basis of their functional groups. All the fungicidal mixing components mentioned in classes (1) to (15), as the case may be, may include tautomeric forms.

1) Ergosterol biosynthesis inhibitors, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulphate, (1.012) ipconazole, (1.013) metconazole, (1.014) myclobutanil, (1.015) paclobutrazol, (1.016) prochloraz, (1.017) propiconazole, (1.018) prothioconazole, (1.019) pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1 S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5 S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulphanyl)-1-{[3-(2-chlorophenyl)-2-(2,4-difluorophenyl)oxiran-2-yl]methyl}-1H-1,2,4-triazole, (1.061) 5-(allylsulphanyl)-1-{[rel(2R,3R)-3-(2-chlorophenyl)-2-(2,4-difluorophenyl)oxiran-2-yl]methyl}-11H-1,2,4-triazole, (1.062) 5-(allylsulphanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulphanyl}phenyl)-N-ethyl-N-methylimidoformamide, (1.064) N′-(2,5-dimethyl-4-{[3-(2,2,2-trifluoroethoxy)phenyl]sulphanyl}phenyl)-N-ethyl-N-methylimidoformamide, (1.065) N′-(2,5-dimethyl-4-{[3-(2,2,3,3-tetrafluoropropoxy)phenyl]sulphanyl}phenyl)-N-ethyl-N-methylimidoformamide, (1.066) N′-(2,5-dimethyl-4-{[3-(pentafluoroethoxy)phenyl]sulphanyl}phenyl)-N-ethyl-N-methylimidoformamide, (1.067) N′-(2,5-dimethyl-4-{3-[(1,1,2,2-tetrafluoroethyl)sulphanyl]phenoxy}phenyl)-N-ethyl-N-methylimidoformamide, (1.068) N′-(2,5-dimethyl-4-{3-[(2,2,2-trifluoroethyl)sulphanyl]phenoxy}phenyl)-N-ethyl-N-methylimidoformamide, (1.069) N′-(2,5-dimethyl-4-{3-[(2,2,3,3-tetrafluoropropy 1)sulphanyl]phenoxy}phenyl)-N-ethyl-N-methylimidoformamide, (1.070) N′-(2,5-dimethyl-4-{3-[(pentafluoroethyl)sulphanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulphanyl}-2,5-dimethylphenyl)-N-ethyl-N-methylimidoformamide, (1.073) N′-(4-{3-[(difluoromethyl)sulphanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1 S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl-N}-ethyl-N-methylimidoformamide, (1.080) N′-{5-bromo-6-[1-(3,5-difluorophenyl)ethoxy]-2-methylpyridin-3-yl}-N-ethyl-N-methylimidoformamide.

2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1Hpyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1Hpyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)N-cyclopropyl-3-(difluoromethyl)-5-fluoro-1-methyl-1H-pyrazole-4-carboxamide, (2.038)N-(5-chloro-2-isopropylbenzyl)-N-cyclopropyl-3-(difluoromethyl)-5-fluoro-1-methyl-1H-pyrazole-4-carboxamide, (2.039)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041)N-[1-(2,4-dichlorophenyl)-1-methoxypropan-2-yl]-3-(difluoromethyl)-1-methyl-1H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044)N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-1H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1Hpyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide.

3) Inhibitors of the respiratory chain in complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)N,3-dimethylpent-3-enamide, (3.023) (2R)-2-{2-[(2,5-dimethylphenoxy)methyl]phenyl}-2-methoxyN-methylacetamide, (3.024) (2S)-2-{2-[(2,5-dimethylphenoxy)methyl]phenyl}-2-methoxy-N-methylacetamide, (3.025) (3 S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide.

4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.

5) Compounds having capacity for multisite activity, for example (5.001) Bordeaux mixture, (5.002) captafol, (5.003) captan, (5.004) chlorthalonil, (5.005) copper hydroxide, (5.006) copper naphthenate, (5.007) copper oxide, (5.008) copper oxychloride, (5.009) copper(2+) sulphate, (5.010) dithianon, (5.011) dodin, (5.012) folpet, (5.013) mancozeb, (5.014) maneb, (5.015) metiram, (5.016) zinc metiram, (5.017) copper oxine, (5.018) propineb, (5.019) sulphur and sulphur preparations including calcium polysulphide, (5.020) thiram, (5.021) zineb, (5.022) ziram.

6) Compounds capable of triggering host defence, for example (6.001) acibenzolar-S-methyl, (6.002) isotianil, (6.003) probenazole, (6.004) tiadinil.

7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

(8) ATP production inhibitors, for example (8.001) silthiofam.

9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Lipid and membrane synthesis inhibitors, for example (10.001) propamocarb, (10.002) propamocarb hydrochloride, (10.003) tolclofos-methyl.

11) Melanin biosynthesis inhibitors, for example (11.001) tricyclazole, (11.002) 2,2,2-trifluoroethyl {3-methyl-1-[(4-methylbenzoyl)amino]butan-2-yl}carbamate.

12) Nucleic acid synthesis inhibitors, for example (12.001) benalaxyl, (12.002) benalaxyl-M (kiralaxyl), (12.003) metalaxyl, (12.004) metalaxyl-M (mefenoxam).

13) Signal transduction inhibitors, for example (13.001) fludioxonil, (13.002) iprodione, (13.003) procymidone, (13.004) proquinazid, (13.005) quinoxyfen, (13.006) vinclozolin.

14) Compounds that can act as uncouplers, for example (14.001) fluazinam, (14.002) meptyldinocap.

15) Further compounds, for example (15.001) abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetylaluminium, (15.013) fosetyl-calcium, (15.014) fosetyl-sodium, (15.015) methyl isothiocyanate, (15.016) metrafenon, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphonic acid and salts thereof, (15.026) propamocarb-fosetylate, (15.027) pyriofenone (chlazafenone) (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5 S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulphonate, (15.045) 2-phenylphenol and salts thereof, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′-phenyl-N′-(prop-2-yn-1-yl)thiophene 2-sulphonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulphate (2:1), (15.061) tert-butyl {6-[({[(1-methyl-1H-tetrazol-5-yl)(phenyl)methylene]amino}oxy)methyl]pyridin-2-yl}carbamate.

### Biological Pesticides as Mixing Components

The compounds of the formula (I) can be combined with biological pesticides.

Biological pesticides especially include bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (new: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, bell peppers, cucumbers, melons, carrots, water melons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The treatment according to the invention of the plants and parts of plants with the compounds of the formula (I) is effected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better capability for storage and/or processability of the harvested products. Further and particularly emphasized examples of such properties are increased resistance of the plants to animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants to phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain herbicidally active compounds, for example imidazolinones, sulphonylureas, glyphosate or phosphinothricin (for example the “PAT” gene). The genes which impart the desired properties (“traits”) in question may also be present in combinations with one another in the transgenic plants. Examples of transgenic plants mentioned include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (the fruits being apples, pears, citrus fruits and grapevines), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection—Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, meaning that the compounds of the formula (I) are applied to the foliage, in which case the treatment frequency and the application rate should be adjusted according to the level of infestation with the pest in question.

In the case of systemically active compounds, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) according to the invention are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and also the germinating plant with a minimum expenditure on pesticides.

The present invention therefore in particular also relates to a method for the protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) according to the invention for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occur when a compound of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for the protection of seed of any plant variety which is used in agriculture, in greenhouses, in forests or in horticulture. More particularly, this is the seed of cereals (for example wheat, barley, rye, millet and oats), maize, cotton, soya beans, rice, potatoes, sunflowers, coffee, tobacco, canola, oilseed rape, beets (for example sugar beets and fodder beets), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape, vegetables and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water, until it reaches a certain stage of the rice embryo (“pigeon breast stage”) which results in stimulation of germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active ingredients which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing the compounds of the formula (I) with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical ingredients. Usable with preference are alkyl naphthalenesulphonates, such as diisopropyl or diisobutyl naphthalenesulphonates.

Suitable dispersants and/or emulsifiers which may be present in the seed-dressing formulations usable in accordance with the invention are all nonionic, anionic and cationic dispersants customary for the formulation of active agrochemical ingredients. Nonionic or anionic dispersants or mixtures of nonionic or anionic dispersants can be used with preference. Suitable nonionic dispersants especially include ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate-formaldehyde condensates.

Antifoams which may be present in the seed-dressing formulations usable in accordance with the invention are all foam-inhibiting substances customary for the formulation of active agrochemical ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz- and Schadlingsbekaimpfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For the treatment of seed with the seed-dressing formulations usable in accordance with the invention, or the use forms prepared therefrom through the addition of water, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasite” includes especially helminths and protozoa, such as coccidia. Ectoparasites are typically and preferably arthropods, especially insects or acarids.

In the field of veterinary medicine, the compounds of the formula (I) having favourable endotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds; reptiles, amphibians or aquarium fish.

In a specific embodiment, the compounds of the formula (I) are administered to mammals.

In another specific embodiment, the compounds of the formula (I) are administered to birds, namely caged birds or particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” in the present context means that the compounds of the formula (I) are effective in reducing the incidence of the particular parasite in an animal infected with such parasites to an innocuous degree. More specifically, “controlling” in the present context means that the compounds of the formula (I) kill the respective parasite, inhibit its growth, or inhibit its proliferation.

The arthropods include, for example, but are not limited to,

from the order of Anoplurida, for example, Haematopinus spp., Linognathus spp., Pediculus spp., Phtirus spp. and Solenopotes spp.;
from the order of Mallophagida and the suborders Amblycerina and Ischnocerina, for example, Bovicola spp., Damalina spp., Felicola spp.; Lepikentron spp., Menopon spp., Trichodectes spp., Trimenopon spp., Trinoton spp., Werneckiella spp;
from the order of Diptera and the suborders Nematocerina and Brachycerina, for example, Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.;
from the order of Siphonapterida, for example, Ceratophyllus spp., Ctenocephalides spp., Pulex spp., Tunga spp., Xenopsylla spp.;
from the order of Heteropterida, for example, Cimex spp., Panstrongylus spp., Rhodnius spp., Triatoma spp.; and also nuisance and hygiene pests from the order Blattarida.

In addition, in the case of the arthropods, mention should be made by way of example, without limitation, of the following Acari:

from the subclass of Acari (Acarina) and the order of Metastigmata, for example from the family of Argasidae such as Argas spp., Ornithodorus spp., Otobius spp., from the family of Ixodidae such as Amblyomma spp., Dermacentor spp., Haemaphysalis spp., Hyalomma spp., Ixodes spp., Rhipicephalus (Boophilus) spp., Rhipicephalus spp. (the original genus of multi-host ticks); from the order of Mesostigmata such as Dermanyssus spp., Ornithonyssus spp., Pneumonyssus spp., Raillietia spp., Sternostoma spp., Tropilaelaps spp., Varroa spp.; from the order of the Actinedida (Prostigmata), for example, Acarapis spp., Cheyletiella spp., Demodex spp., Listrophorus spp., Myobia spp., Neotrombicula spp., Ornithocheyletia spp., Psorergates spp., Trombicula spp.; and from the order of the Acaridida (Astigmata), for example, Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Examples of parasitic protozoa include, but are not limited to:

Mastigophora (Flagellata), such as:
Metamonada: from the order of Diplomonadida, for example, Giardia spp., Spironucleus spp.
Parabasala: from the order of Trichomonadida, for example, Histomonas spp., Pentatrichomonas spp., Tetratrichomonas spp., Trichomonas spp., Tritrichomonas spp.
Euglenozoa: from the order of Trypanosomatida, for example, Leishmania spp., Trypanosoma spp.
Sarcomastigophora (Rhizopoda) such as Entamoebidae, for example Entamoeba spp., Centramoebidae, for example Acanthamoeba sp., Euamoebidae, e.g. Hartmanella sp.
Alveolata such as Apicomplexa (Sporozoa): e.g. Cryptosporidium spp.; from the order of Eimeriida, for example, Besnoitia spp., Cystoisospora spp., Eimeria spp., Hammondia spp., Isospora spp., Neospora spp., Sarcocystis spp., Toxoplasma spp.; from the order of Adeleida, for example, Hepatozoon spp., Klossiella spp.; from the order of Haemosporida, for example, Leucocytozoon spp., Plasmodium spp.; from the order of Piroplasmida, for example, Babesia spp., Ciliophora spp., Echinozoon spp., Theileria spp.; from the order of Vesibuliferida, for example, Balantidium spp., Buxtonella spp.
Microspora such as Encephalitozoon spp., Enterocytozoon spp., Globidium spp., Nosema spp., and also, for example, Myxozoa spp.

The helminths that are pathogenic to humans or animals include, for example, Acanthocephala, nematodes, Pentastoma and Platyhelminthes (e.g. Monogenea, cestodes and trematodes).

Exemplary helminths include, but are not limited to:

Monogenea: e.g. Dactylogyrus spp., Gyrodactylus spp., Microbothrium spp., Polystoma spp., Troglecephalus spp.;
Cestodes: from the order of Pseudophyllidea, for example: Bothridium spp., Diphyllobothrium spp., Diplogonoporus spp., Ichthyobothrium spp., Ligula spp., Schistocephalus spp., Spirometra spp.

From the order of Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.

Trematodes: from the class of Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Ornithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes: from the order of Trichinellida, for example: Capillaria spp., Trichinella spp., Trichomosoides spp., Trichuris spp.

From the order of Tylenchida, for example: Micronema spp., Parastrangyloides spp., Strongyloides spp.

From the order of Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Ornithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.

From the order of Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dra-cunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.

Acanthocephala: from the order of Oligacanthorhynchida, for example: Macracanthorhynchus spp., Prosthenorchis spp.; from the order of Moniliformida, for example: Moniliformis spp.

From the order of Polymorphida, for example: Filicollis spp.; from the order of Echinorhynchida, for example Acanthocephalus spp., Echinorhynchus spp., Leptorhynchoides spp.

Pentastoma: from the order of Porocephalida, for example, Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic, metaphylactic or therapeutic.

Thus, one embodiment of the present invention refers to the compounds of the formula (I) for use as a medicament.

A further aspect relates to the compounds of the formula (I) for use as an antiendoparasitic agent.

A further specific aspect of the invention relates to the compounds of the formula (I) for use as an antithelminthic agent, especially for use as a nematicide, platyhelminthicide, acanthocephalicide or pentastomicide.

A further specific aspect of the invention relates to the compounds of the formula (I) for use as an antiprotozoic agent.

A further aspect relates to the compounds of the formula (I) for use as an antiectoparasitic agent, especially an arthropodicide, very particularly an insecticide or an acaricide.

Further aspects of the invention are veterinary medicine formulations comprising an effective amount of at least one compound of the formula (I) and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a veterinary medicine formulation as described here, which comprises the step of mixing at least one compound of the formula (I) with pharmaceutically acceptable excipients and/or auxiliaries, especially with pharmaceutically acceptable excipients used conventionally in veterinary medicine formulations and/or auxiliaries used conventionally in veterinary medicine formulations.

Another specific aspect of the invention is veterinary medicine formulations selected from the group of ectoparasiticidal and endoparasiticidal formulations, especially selected from the group of anthelmintic, antiprotozoic and arthropodicidal formulations, very particularly selected from the group of nematicidal, platyhelminthicidal, acanthocephalicidal, pentastomicidal, insecticidal and acaricidal formulations, according to the aspects mentioned, and methods for production thereof.

Another aspect relates to a method for treatment of a parasitic infection, especially an infection caused by a parasite selected from the group of the ectoparasites and endoparasites mentioned here, by use of an effective amount of a compound of the formula (I) in an animal, especially a nonhuman animal, having a need therefor.

Another aspect relates to a method for treatment of a parasitic infection, especially an infection caused by a parasite selected from the group of the ectoparasites and endoparasites mentioned here, by use of a veterinary medicine formulation as defined here in an animal, especially a nonhuman animal, having a need therefor.

Another aspect relates to the use of the compounds of the formula (I) in the treatment of a parasite infection, especially an infection caused by a parasite selected from the group of the ectoparasites and endoparasites mentioned here, in an animal, especially a nonhuman animal.

In the present context of animal health or veterinary medicine, the term “treatment” includes prophylactic, metaphylactic and therapeutic treatment.

In a particular embodiment, in this way, mixtures of at least one compound of the formula (I) with other active compounds, especially with endo- and ectoparasiticides, are provided for the field of veterinary medicine.

In the field of animal health, “mixture” means not just that two (or more) different active compounds are formulated in a common formulation and are correspondingly employed together, but also relates to products comprising formulations separated for each active compound. Accordingly, when more than two active ingredients are to be employed, all active ingredients can be formulated in a common formulation or all active ingredients can be formulated in separate formulations; likewise conceivable are mixed forms in which some of the active ingredients are formulated together and some of the active ingredients are formulated separately. Separate formulations allow the separate or successive application of the active ingredients in question.

The active compounds specified here by their “common names” are known and are described, for example, in the “Pesticide Manual” (see above) or can be searched for on the Internet (e.g.: http://www.alanwood.net/pesticides).

Illustrative active ingredients from the group of the ectoparasiticides as mixing components, without any intention that this should constitute a restriction, include the insecticides and acaricides listed in detail above. Further usable active ingredients are listed below in accordance with the abovementioned classification based on the current IRAC Mode of Action Classification Scheme: (1) acetylcholinesterase (AChE) inhibitors; (2) GABA-gated chloride channel blockers; (3) sodium channel modulators; (4) nicotinic acetylcholine receptor (nAChR) competitive modulators; (5) nicotinic acetylcholine receptor (nAChR) allosteric modulators; (6) glutamate-gated chloride channel (GluCl) allosteric modulators; (7) juvenile hormone mimetics; (8) miscellaneous non-specific (multi-site) inhibitors; (9) chordotonal organ modulators; (10) mite growth inhibitors; (12) inhibitors of mitochondrial ATP synthase, such as ATP disruptors; (13) uncouplers of oxidative phosphorylation via disruption of the proton gradient; (14) nicotinic acetylcholine receptor channel blockers; (15) inhibitors of chitin biosynthesis, type 0; (16) inhibitors of chitin biosynthesis, type 1; (17) moulting disruptors (especially in Diptera); (18) ecdysone receptor agonists; (19) octopamine receptor agonists; (21) mitochondrial complex I electron transport inhibitors; (25) mitochondrial complex II electron transport inhibitors; (20) mitochondrial complex III electron transport inhibitors; (22) voltage-dependent sodium channel blockers; (23) inhibitors of acetyl CoA carboxylase; (28) ryanodine receptor modulators;

active ingredients having unknown or non-specific mechanisms of action, e.g. fentrifanil, fenoxacrim, cycloprene, chlorobenzilate, chlordimeform, flubenzimin, dicyclanil, amidoflumet, quinomethionat, triarathene, clothiazoben, tetrasul, potassium oleate, petroleum, metoxadiazone, gossyplur, flutenzine, brompropylate, cryolite;
compounds from other classes, for example butacarb, dimetilan, cloethocarb, phosphocarb, pirimiphos(-ethyl), parathion(-ethyl), methacrifos, isopropyl o-salicylate, trichlorfon, sulprofos, propaphos, sebufos, pyridathion, prothoate, dichlofenthion, demeton-S-methyl sulphone, isazofos, cyanofenphos, dialifos, carbophenothion, autathiofos, aromfenvinfos(-methyl), azinphos(-ethyl), chlorpyrifos(-ethyl), fosmethilan, iodofenphos, dioxabenzofos, formothion, fonofos, flupyrazofos, fensulfothion, etrimfos;
organochlorine compounds, for example camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-)metofluthrin, profluthrin, flufenprox, flubrocythrinate, fubfenprox, fenfluthrin, protrifenbut, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated hydrocarbon compounds (HCHs),
neonicotinoids, e.g. nithiazine
dicloromezotiaz, triflumezopyrim
macrocyclic lactones, e.g. nemadectin, ivermectin, latidectin, moxidectin, selamectin, eprinomectin, doramectin, emamectin benzoate; milbemycin oxime
triprene, epofenonane, diofenolan;
biologicals, hormones or pheromones, for example natural products, e.g. thuringiensin, codlemone or neem components
dinitrophenols, e.g. dinocap, dinobuton, binapacryl;
benzoylureas, e.g. fluazuron, penfluron,
amidine derivatives, e.g. chlormebuform, cymiazole, demiditraz
beehive varroa acaricides, for example organic acids, e.g. formic acid, oxalic acid.

Illustrative active ingredients from the group of the endoparasiticides, as mixing components, include, but are not limited to, active anthelmintic ingredients and active antiprotozoic ingredients.

The anthelmintically active compounds include but are not limited to the following nematicidally, trematicidally and/or cestocidally active compounds:

from the class of the macrocyclic lactones, for example: eprinomectin, abamectin, nemadectin, moxidectin, doramectin, selamectin, lepimectin, latidectin, milbemectin, ivermectin, emamectin, milbemycin;
from the class of the benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole sulphoxide, albendazole, flubendazole;
from the class of the depsipeptides, preferably cyclic depsipeptides, especially 24-membered cyclic depsipeptides, for example: emodepside, PF1022A;
from the class of the tetrahydropyrimidines, for example: morantel, pyrantel, oxantel;
from the class of the imidazothiazoles, for example: butamisole, levamisole, tetramisole;
from the class of the aminophenylamidines, for example: amidantel, deacylated amidantel (dAMD), tribendimidine;
from the class of the aminoacetonitriles, for example: monepantel;
from the class of the paraherquamides, for example: paraherquamide, derquantel;
from the class of the salicylanilides, for example: tribromsalan, bromoxanide, brotianide, clioxanide, closantel, niclosamide, oxyclozanide, rafoxanide;
from the class of the substituted phenols, for example: nitroxynil, bithionol, disophenol, hexachlorophen, niclofolan, meniclopholan;
from the class of the organophosphates, for example: trichlorfon, naphthalofos, dichlorvos/DDVP, crufomate, coumaphos, haloxon;
from the class of the piperazinones/quinolines, for example: praziquantel, epsiprantel;
from the class of the piperazines, for example: piperazine, hydroxyzine;
from the class of the tetracyclines, for example: tetracycline, chlorotetracycline, doxycycline, oxytetracycline, rolitetracycline;
from various other classes, for example: bunamidine, niridazole, resorantel, omphalotin, oltipraz, nitroscanate, nitroxynil, oxamniquin, mirasan, miracil, lucanthon, hycanthon, hetolin, emetin, diethylcarbamazine, dichlorophen, diamfenetide, clonazepam, bephenium, amoscanate, clorsulon.

Active antiprotozoic ingredients include, but are not limited to, the following active ingredients:

from the class of the triazines, for example: diclazuril, ponazuril, letrazuril, toltrazuril;
from the class of polyether ionophores, for example: monensin, salinomycin, maduramicin, narasin;
from the class of the macrocyclic lactones, for example: milbemycin, erythromycin;
from the class of the quinolones, for example: enrofloxacin, pradofloxacin;
from the class of the quinines, for example: chloroquin;
from the class of the pyrimidines, for example: pyrimethamine;
from the class of the sulphonamides, for example: sulfaquinoxaline, trimethoprim, sulfaclozin;
from the class of the thiamines, for example: amprolium;
from the class of the lincosamides, for example: clindamycin;
from the class of the carbanilides, for example: imidocarb;
from the class of the nitrofurans, for example: nifurtimox;
from the class of the quinazolinone alkaloids, for example: halofuginone;
from various other classes, for example: oxamniquin, paromomycin;
from the class of the vaccines or antigens from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All the mixing components mentioned, as the case may be, may also form salts with suitable bases or acids if they are capable of doing so on the basis of their functional groups.

### Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) onto a host or after injection into a host (for example malaria parasites by mosquitoes).

Examples of vectors and the diseases or pathogens they transmit are:

1) mosquitoes
Anopheles: malaria, filariasis;
Culex: Japanese encephalitis, filariasis, other viral diseases, transmission of other worms;
Aedes: yellow fever, dengue fever, further viral disorders, filariasis;
Simuliidae: transmission of worms, especially Onchocerca volvulus;
Psychodidae: transmission of leishmaniasis
   2) Lice: skin infections, epidemic typhus;
   3) Fleas: plague, endemic typhus, tapeworms;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;
   6) Ticks: borellioses such as Borrelia bungdorferi sensu lato., Borrelia duttoni, tick-borne encephalitis, Q fever (Coxiella burnetii), babesioses (Babesia canis canis), ehrlichiosis.

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, Psychodidae such as Phlebotomus, Lutzomyia, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

### Protection of Industrial Materials

The compounds of the formula (I) are suitable for protecting industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) take the form of a ready-to-use pesticide, meaning that they can be applied to the material in question without further modifications. Useful further insecticides or fungicides especially include those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active compounds, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids, ticks and mites encountered in enclosed spaces, for example dwellings, factory halls, offices, vehicle cabins, animal breeding facilities. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propellerdriven evaporators, energy-free, or passive, evaporation systems, moth papers, moth bags and moth gels, as granules or dusts, in baits for spreading or bait stations.

### Description of the Processes and Intermediates

By way of example and supplementarily, the preparation of compounds of the formula (I) is illustrated in the formula schemes below. Here, reference is also made to the preparation examples.

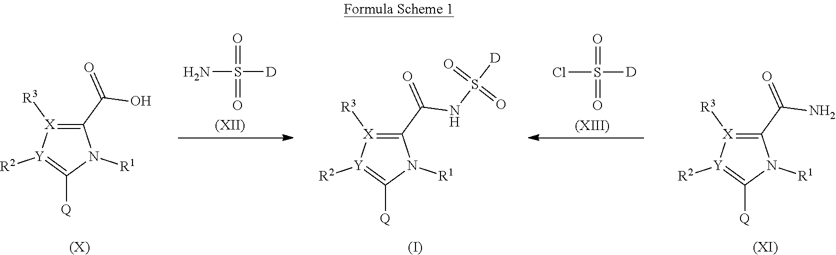

According to formula scheme 1, the compounds of the formula (I) according to the invention are prepared from carboxylic acids of the formula (X) by reaction with a coupling agent and sulphonamides of the formula (XII), see, for example, WO2012/80447, WO2006/114313, WO2015/11082, WO2010/129500, US2008/227769 and WO2009/67108. Alternatively, the compounds of the formula (I) can also be prepared by reacting a carboxamide of the formula (XI) with a sulphonyl chloride of the formula (XIII) in the presence of a base such as, for example, sodium hydride, see, for example, US2004/6143. The required amides of the formula (XI) can be obtained from the acids of the formula (X), for example by reaction with a coupling agent and ammonium acetate, see, for example, U.S. Pat. No. 5,300,498.

The required sulphonamides and sulphonyl chlorides of the formulae (XII) and (XIII) are known or can be prepared by generally known methods. Here, the sulphonamides can be obtained from the sulphonyl chlorides by reaction with ammonia, see WO2014/146490, Eur. J. Med. Chem. 2013, 62, 597-604; Bioorg. Med. Chem. 2005, 13, 7, 2459-2468.

Further examples are:

3-Chlorobenzenesulphonamide: Coll. Czech. Chem. Comm. 1984, 49, 5, 1182-1192
2-Chlorobenzenesulphonyl chloride: U.S. Pat. No. 5,099,025
2-Chloro-5-methoxybenzenesulphonamide: WO2010/129500

### Isopropylsulphonamide: US542803

The required carboxylic acids of the formula (X) are known or can be prepared analogously to generally known processes or by known processes or according to Processes A to G described below.

Examples of known acids of the formula (X):

For 5-methyl-1-[2-(trifluoromethyl)phenyl]-1H-pyrazole-3-carboxylic acid, see Bioorg. Med. Chem. Lett. 2001, Vol 11, #17, 287-2290

For 2-(2-chlorophenyl)-5-methyl-1H-imidazole-4-carboxylic acid, see WO2004/60870 A1 page 50

For 2-(2-chlorophenyl)-1-(4-chlorophenyl)-1H-imidazole-4-carboxylic acid, see Bioorg. Med. Chem. Lett. 2007, Vol 17, #10, 2706-2711

### Process A

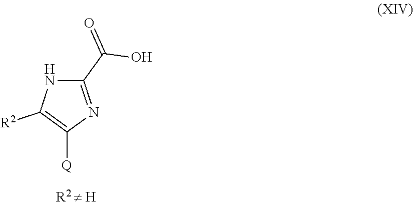

Compounds of the formula (XIV) are prepared according to Formula Scheme 1 from bromides of the formula (XV) by reaction with a boronic acid or a boronic ester of the formula (XVI), a palladium catalyst such as, for example, tetrakis(triphenylphosphine)palladium or (1,1′-bis(diphenylphosphino)ferrocene)palladium dichloride and a base such as, for example, potassium carbonate, in a high-boiling solvent such as, for example, DMF. See, for example, WO2011/149874 A2 or EP2518054 A1.

In Formula Scheme 1, R^{x} can be, for example, H or alkyl (including cyclic). R^{y} can represent, for example, methyl, ethyl, propyl or tert-butyl.

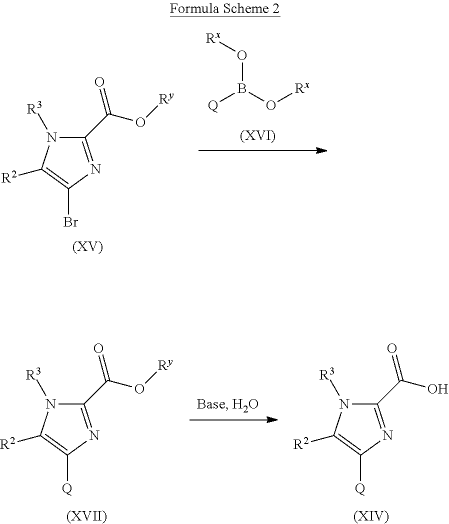

The hydrolysis of the ester to give the target compound XIV is carried out according to generally known conditions (LiOH, H₂O, THF or NaOH, EtOH). In the case of R^{y}=tert-butyl, the ester is hydrolyzed under acidic conditions using, for example, trifluoroacetic acid in dichloromethane.

Boronic acids or boronic esters of the formula (XVI) are either commercially available or can be prepared by known methods.

The bromides of the formula XV required are prepared according to Scheme 2. The starting material of the general formula XVIII is either commercially available or can be prepared by known methods.

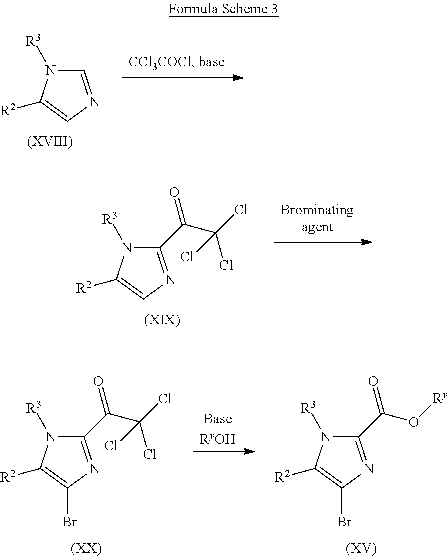

Reaction of an imidazole XVIII with trichloroacetyl chloride, as described in Bioorganic and Medicinal Chemistry Letters, 2008, 18, 4325 or EP2518054 A1, yields intermediate (XIX). This is brominated by reaction with a brominating agent (inter alia N-bromosuccinimide, Br₂/AcOH), as published, for example, in WO2008/85302 A1 or WO2015/25025 A1. The target compound (XV) is obtained by alcoholysis in the presence of a base (see, for example, WO2007/45096 A1, Chemistry—A European Journal, 2003, 9, 3353).

### Process B

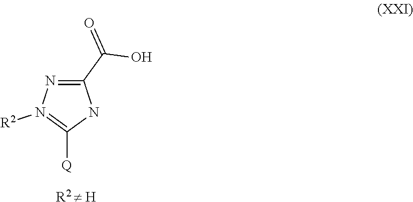

According to Formula Scheme 4, the preparation of compounds of the formula (XXI) is carried out as described in general terms in Tetrahedron Letters, 2012, 53, 6078. In Formula Scheme 4, R^{y} can represent, for example, methyl, ethyl, propyl or tert-butyl.

The starting material of the formula (XXII) is either commercially available or can be prepared by known methods.

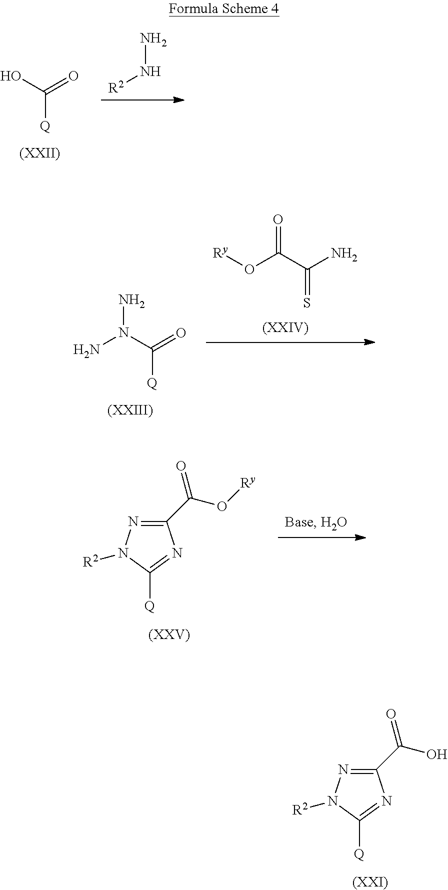

Reaction of the acid XXII with an alkylhydrazine and a suitable coupling agent/catalyst system (e.g. 2,3,4,5,6-pentafluorophenol/1-ethyl-(3-(3-dimethylamino)propyl)carbodiimide hydrochloride) affords intermediate (XXIII), which is cyclized with ethyl amino(thioxo)acetate in the presence of acetic acid in toluene.

The hydrolysis of the ester to give the target compound XXI is carried out according to generally known conditions (LiOH, H₂O, THF or NaOH, EtOH). In the case of R^{y}=tert-butyl, the ester is hydrolyzed under acidic conditions using, for example, trifluoroacetic acid in dichloromethane.

### Process C:

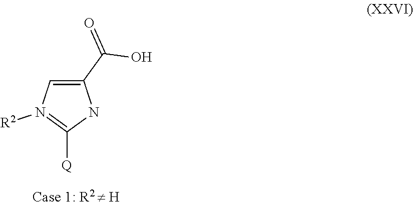

Compounds of the formula (XXVI) are prepared according to Formula Scheme 5. In Formula Scheme 5, R^{x} can be, for example, H or alkyl (including cyclic). R^{y} can represent, for example, methyl, ethyl, propyl or tert-butyl.

The esters XXVII, required as starting material, are either commercially available or can be prepared by esterification of the corresponding acid by known methods.

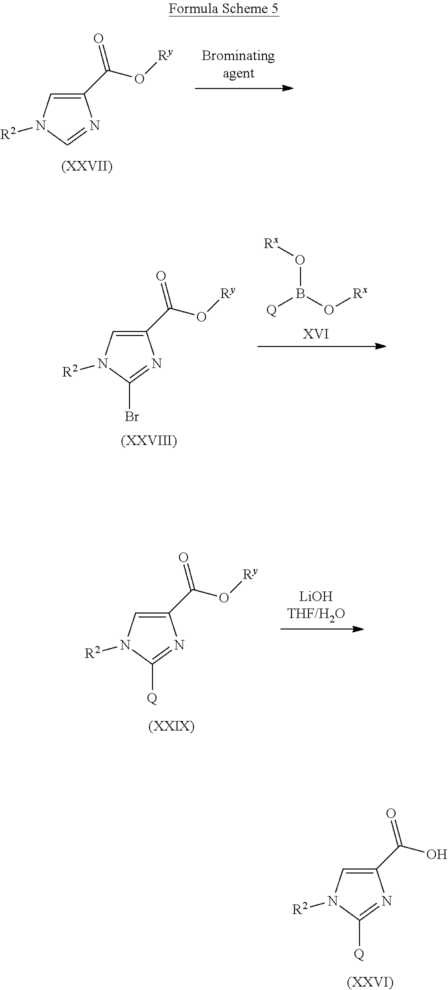

The bromination is carried out using a brominating agent such as, for example, N-bromosuccinimide, analogously to WO2014/191894 A1, 2014.

Bromides of the formula (XXVIII) can then be converted into compounds of the formula XXIX by reaction with a boronic acid or a boronic ester of the formula (XVI), a palladium catalyst such as, for example, tetrakis(triphenylphosphine)palladium or chloro(2-dicyclohexylphosphino-2′,4′,6′-triisopropyl-1,1′-biphenyl)[2-(2′-amino-1,1′-biphenyl)]palladium(II) (XPhos Pd G2) and a base such as, for example, potassium phosphate, in a solvent such as, for example, 1,4-dioxane or THF, under reflux. See, for example, WO2014/115077 A1, 2014 or J. Am. Chem. Soc., 2010, 132, 14073.

Boronic acids or boronic esters of the formula (XVI) are either commercially available or can be prepared by known methods.

The hydrolysis of the ester to give the target compound XXVI is carried out according to generally known conditions (LiOH, H₂O, THF or NaOH, EtOH). In the case of R^{Y}=tert-butyl, the ester is hydrolyzed under acidic conditions using, for example, trifluoroacetic acid in dichloromethane.

### Process D

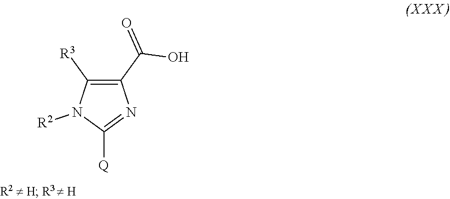

Compounds of the formula (XXX) according to the invention are prepared according to Formula Scheme 6 from bromides of the formula (XXXI) by reaction with a boronic acid, which is commercially available or can be prepared under known conditions, or a boronic ester of the formula (XVI), a palladium catalyst such as, for example, [1,1′-bis(diphenylphosphino)ferrocene]palladium(II) chloride/dichloromethane complex and a base such as, for example, caesium carbonate, in a high-boiling solvent such as, for example, toluene under reflux. See, for example, Journal of Organic Chemistry, 2004, 69, 8829. In Formula Scheme 6, R^{x} can be, for example, H or alkyl (including cyclic). R^{y} can represent, for example, methyl, ethyl, propyl or tert-butyl.

The hydrolysis of the ester to give the target compound XXX is carried out according to generally known conditions (LiOH, H₂O, THF or NaOH, EtOH). In the case of R^{Y}=tert-butyl, the ester is hydrolyzed under acidic conditions using, for example, trifluoroacetic acid in dichloromethane.

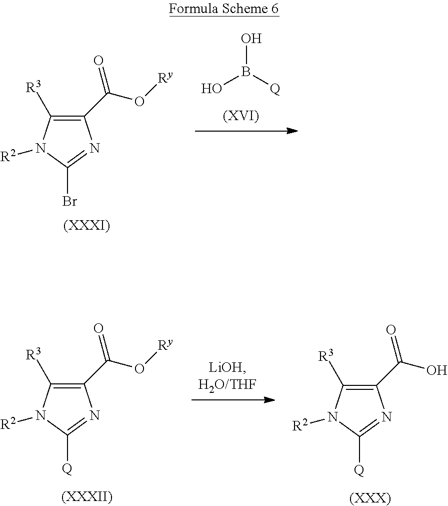

Boronic acids of the formula (XVI) are either commercially available or can be prepared by known methods.

The brominated building block XXXI required is prepared according to Scheme 7.

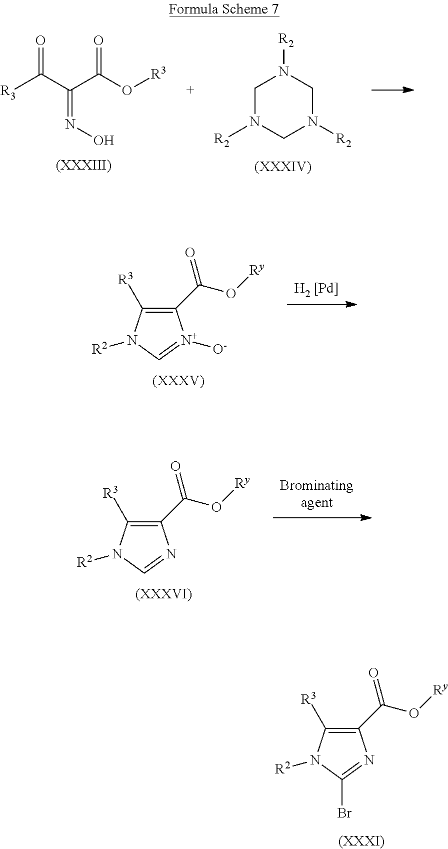

The cyclization of a 1,3,5-trialkyl-1,3,5-triazinane XXXIV with a 2-(hydroxyimino)-3-oxocarboxylic ester XXXIII is described, for example, in Helvetica Chimica Acta, 2008, 91, 1916. Subsequent reduction can be carried out by addition of a suitable heterogenous catalyst such as, for example, Raney nickel in a suitable solvent such as, for example, ethanol. The reaction is preferably carried out at room temperature under elevated pressure (for example 2 bar). Analogous reactions are described, for example, in Tetrahedron Asymmetry, 2013, 24, 958.

The imidazole of the formula XXXVI can be converted into the required building block XXXI for example analogously to EP2518054 A1, 2012 by bromination with N-bromosuccinimide in acetonitrile.

### Process E

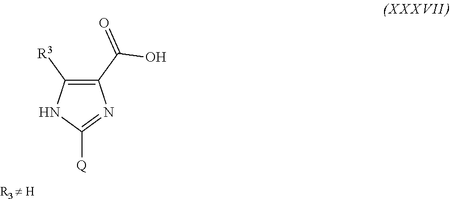

Compounds of the formula (XXXVII) according to the invention are prepared according to Formula Scheme 8. In Formula Scheme 8, R^{y} can represent, for example, methyl, ethyl, propyl or tert-butyl.

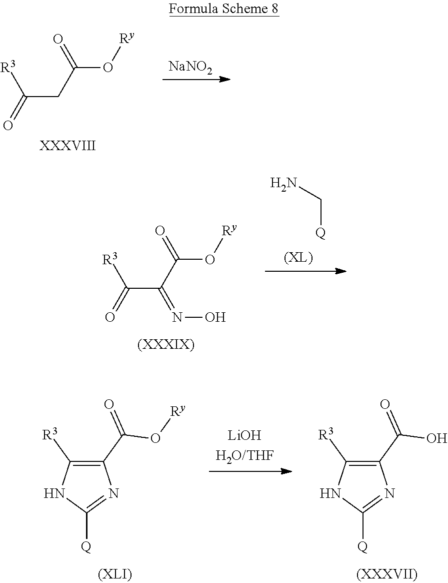

According to WO2011/119777 A2, 2011 or US2012/238599 A1, 2012, an α-keto ester XXXVIII, which is commercially available or can be prepared according to generally known conditions, is converted into an oxime of the general formula XXXIX by reaction with sodium nitrite and acetic acid in water. This can be cyclized by heating with an amine in a suitable solvent (for example acetonitrile or toluene) analogously to WO2005/99705 A2, 2005 or U.S. Pat. No. 6,288,061 B1, 2001, to give an imidazole of the formula XLI.

The hydrolysis of the ester to give the target compound XXXVII is carried out according to generally known conditions (LiOH, H₂O, THF or NaOH, EtOH). In the case of R^{y}=tert-butyl, the ester is hydrolyzed under acidic conditions using, for example, trifluoroacetic acid in dichloromethane. Process F:

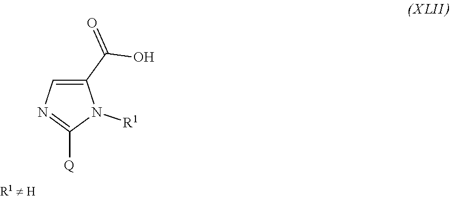

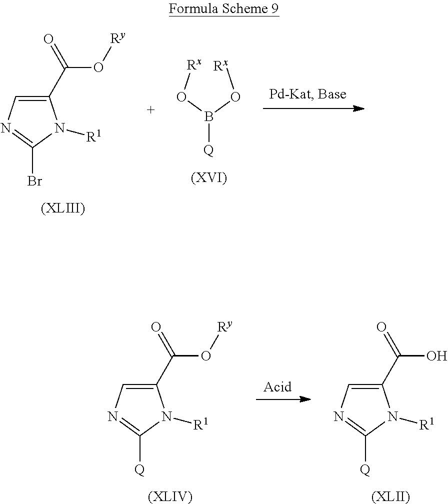

Compounds of the formula (XLII) according to the invention are prepared starting with 2-haloimidazoles of the formula (XLIII) by reaction with a boronic acid or boronic ester of the formula (XVI). In Formula Scheme 9, R^{x} can be, for example, H or alkyl (including cyclic). R^{y} can represent, for example, methyl, ethyl, propyl or tert-butyl.

For example, reaction of a bromide of the formula (XLIII) with a boronic ester of the formula (XVI) in the presence of a palladium catalyst such as, for example, tetrakis(triphenylphosphine)palladium or [1,1′-bis(diphenylphosphino)ferrocene]dichloropalladium and an auxiliary base such as potassium carbonate or caesium carbonate gives the compounds of the formula (XLIV). For methyl 2-bromo-3-methyl-3H-imidazole-4-carboxylate, for example, see: US2009/23707 A1. In the case that R^{y}=tbutyl, the compounds of the formula (XLII) can be released from these esters of the formula (XLIV) by reaction with an acid such as, for example, trifluoroacetic acid. For tert-butyl 2-(2-chlorophenyl)-5-methyl-1-(4-nitrophenyl)-1H-imidazole-4-carboxylate, for example, see: WO2005/99705 A2.

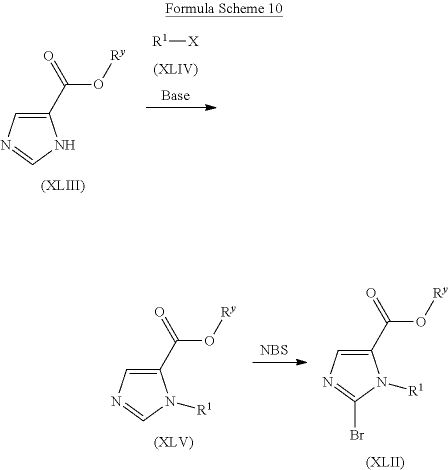

The compounds of the formula (XLII) according to the invention are prepared from imidazoles of the formula (XLIII) by reaction with halides of the formula (XLIV) and a strong or weak base such as, for example, sodium hydride or potassium carbonate. For ethyl imidazole-5-carboxylate, for example, see: Green Chemistry 2013, 15, 2740-2746. Imidazoles of the formula (XLV) can be converted with N-bromosuccinimide into compounds of the formula (XLII). For tert-butyl 1-methyl-1H-imidazole-5-carboxylate, for example, see: Journal of Chemical Research—Part S, 2000, 5, 230-231.

### Process G

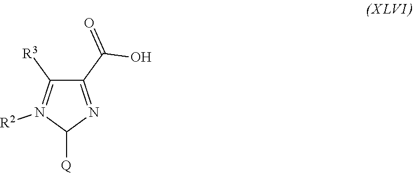

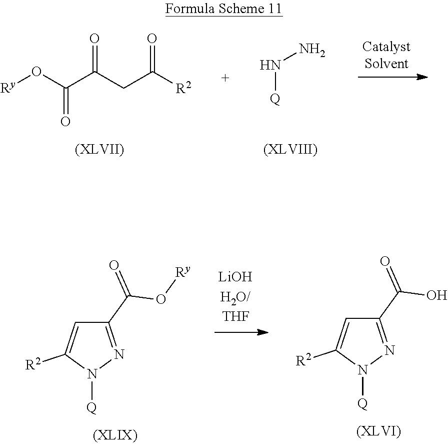

Compounds of the formula (XLVI) according to the invention are prepared from a corresponding arylhydrazine XLVII and a diketone XLVII. The cyclization can be catalyzed by base or acid, as described, for example, in US2007/287734 A1, 2007 (base: sodium hydroxide in ethanol) or U.S. Pat. No. 6,020,357 A1, 2000 (acid: para-toluenesulphonic acid, in ethanol).

The hydrolysis of the ester to give the target compound XLVI is carried out according to generally known conditions (LiOH, H₂O, THF or NaOH, EtOH).

The compounds of the formula (XLVI) according to the invention where R²=alkoxy are prepared from the esters (XLIXb) which are obtained according to Formula Scheme 11a. To this end, an arylhydrazine of the formula (XLVIII) is reacted with a diazodicarboxylate, see, for example, US2004/248881 page 25-26. The ester (XLIXa) obtained is then alkylated with an alkylating agent such as methyl iodide in the presence of a base such as potassium carbonate to give the ester of the formula (XLIXb); see, for example, US2014/315934 § 0919, which can then be reacted further in accordance with the route described above.

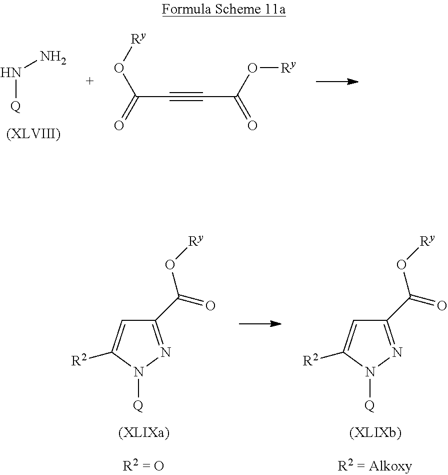

### Process H

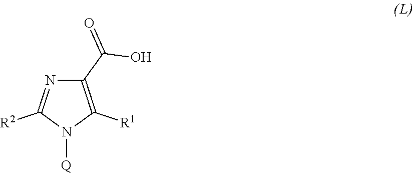

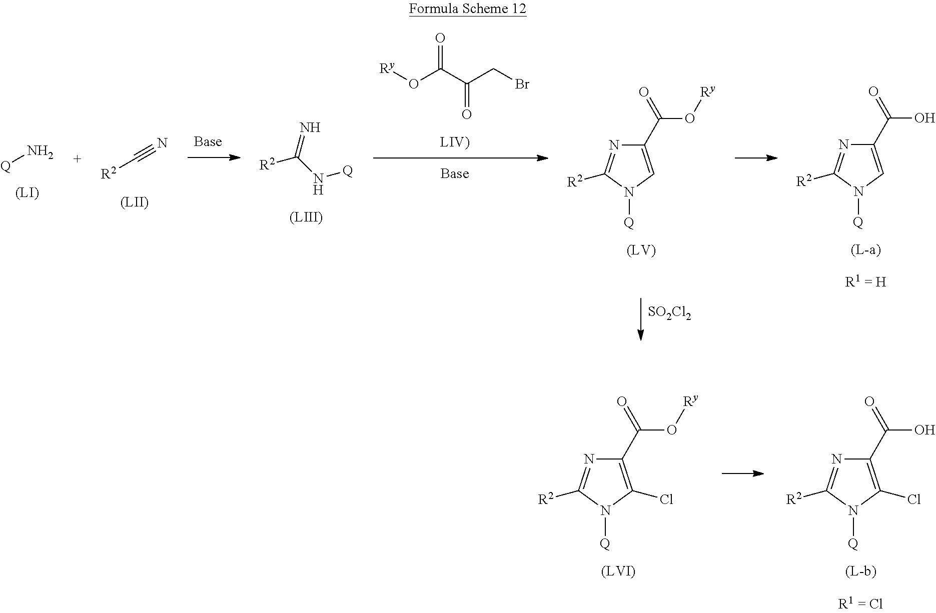

Compounds of the formula (L) are prepared from a corresponding nitrile LII and an amine LI which are linked with the aid of a base such as, for example, NaHMDS to give an amidine LIII. See, for example, Journal of Medicinal Chemistry, 2005, (48), 1823. The amidine can be cyclized, for example with base catalysis (e.g. using NaHCO₃) in a polar-protic solvent such as isopropanol using an abromoketone LIV, to give the imidazole LV. See US2004/122074 A1, 2004.

The resulting imidazole of type LV can either be hydrolyzed directly using a base to give L-a (as already described repeatedly above), or it is first chlorinated by reaction with SO₂Cl₂ or oxalyl chloride to give a precursor of type LVI, which can then for its part be hydrolysed to L-b. For the chlorination, see, for example, WO2005/99705 A2, 2005 or EP2196459 A1, 2010.

### Process I

If Q is a pyridine derivative such as, for example, in the case Q=(VIz), (VIa1), (VIa6), (VIa17), the preparation of compounds of the formula (XLII, R¹≠H) according to the invention is carried out in accordance with Formula Scheme 13 and Formula Scheme 14 by two possible processes.

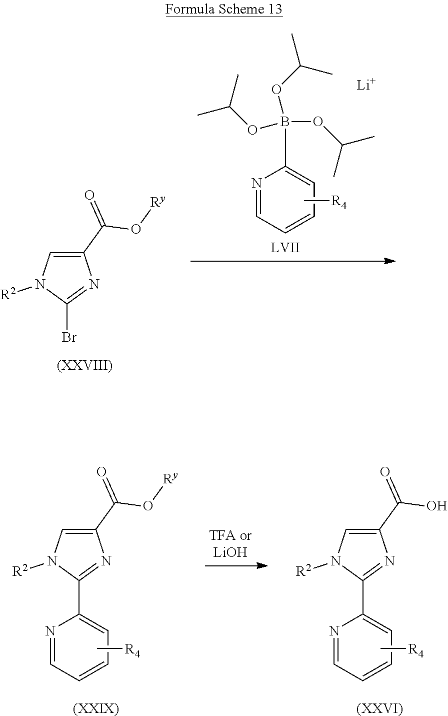

Similar to Process C, compounds of the formula (XXVIII) can, instead of a boronic acid, be reacted with a triisopropoxy borate of the formula (LVII) in the presence of a palladium catalyst (consisting of palladium salt and optionally a further ligand), a copper salt and a base. A suitable reaction system is, for example, the combination of palladium acetate, 1,1′-bis(diphenylphosphino)ferrocene, copper(I) iodide and caesium carbonate, as described in Org. Lett., 2009, (11), 345. The reaction can be carried out in a solvent that is inert under the reaction conditions such as, for example, dimethylformamide. The reaction is typically carried out within a temperature range of 50° C.-150° C.

The general preparation of triisopropoxy borates of the formula (LVII) is described in Tet. Lett. 2012 (53), 4873 or WO2011103435A2.

The hydrolysis of the ester to give the target compound XXVI is carried out according to generally known conditions (LiOH, water/THF or NaOH, EtOH). In the case of Ry=tert-butyl, the ester is hydrolyzed under acidic conditions using, for example, trifluoroacetic acid in dichloromethane.

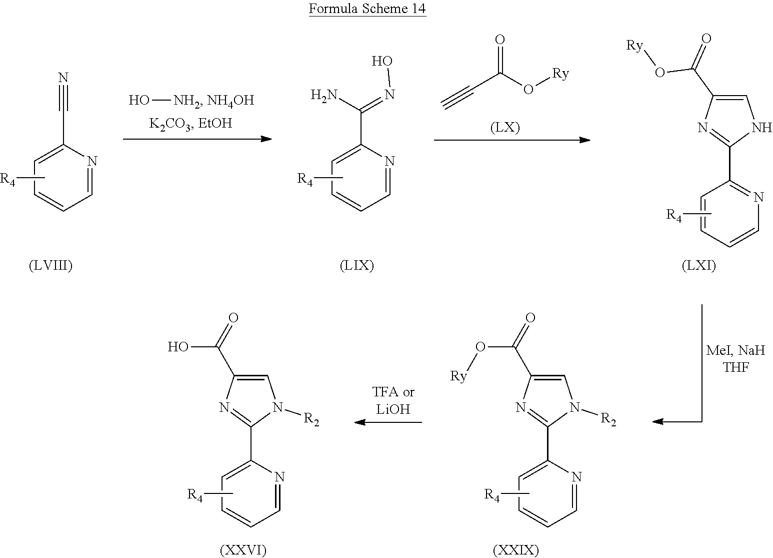

Alternatively to the process described above, compounds of the formula (XLII, R¹≠H) can be prepared from nitriles of the formula (LVIII).

In the first step, according to Bioorganic and Medicinal Chemistry Letters, 2012, vol. 22, #22 p. 6974-6979, 6 or WO2007/75749 A2, 2007, compound (LVIII) is converted by reaction with hydroxylamine in the presence of a base into the amidoxime (LIX). Suitable bases are, for example, K2CO3 or NaOH. The reaction can be carried out in a solvent such as, for example, ethanol, methanol or water.

In a second step, (LIX) is cyclized with a propanoate (LX) to give the imidazole (LXI), see, for example, U.S. Pat. No. 4,853,383 A1, 1989, U.S. Pat. No. 6,492,516 B1, 2002. The reaction is usually carried out at 50-200° C. Suitable solvents are alcohols such as methanol or high-boiling solvents such as diphenyl ether.

In the third step, with the aid of an alkylating agent and in the presence of a base, compound (LXI) is converted into an imidazole (XXIX). Suitable alkylating agents are, for example, alkyl halides such as methyl iodide. Suitable bases are sodium hydride, potassium carbonate, potassium tert-butoxide. See WO2008/84218 A1, 2008, WO2007/113276 A1, 2007 WO2016/46230 A1, 2016. The reaction can be carried out in a solvent that is inert under the reaction conditions such as, for example, THF.

The hydrolysis of the ester (XXIX) to give the target compound (XXVI) is carried out according to generally known conditions (LiOH, water/THF or NaOH, EtOH). In the case of Ry=tert-butyl, the ester is hydrolyzed under acidic conditions using, for example, trifluoroacetic acid in dichloromethane.

### PREPARATION EXAMPLES

The preparation and use examples which follow illustrate the invention without limiting it.

### Methods

The log P values were determined according to EEC Directive 79/831 Annex V.A8 by HPLC (highperformance liquid chromatography) on a reversed-phase column (C18). Temperature 43° C. The calibration is effected with unbranched alkan-2-ones (having 3 to 16 carbon atoms), for which the log P values are known.

The determination of the M⁺ by LC-MS in the acidic range was carried out at pH 2.7 using the mobile phases 0.1% aqueous formic acid and acetonitrile (contains 0.1% formic acid); linear gradient from 10% acetonitrile to 95% acetonitrile, instrument: Agilent 1100 LC system, Agilent MSD system, HTS PAL.

The determination of the M⁺ by LC-MS in the neutral range was carried out at pH 7.8 using the mobile phases 0.001 molar aqueous ammonium bicarbonate solution and acetonitrile; linear gradient from 10% acetonitrile to 95% acetonitrile.

The log P values for the acidic range (as log P [a]) and/or for the neutral range (as log P [n]) were stated in the tables and preparation examples.

b) The determination of the ¹H NMR data was effected with a Bruker Avance 400 equipped with a sample flow head (capacity 60 μl), with tetramethylsilane as reference (0.0) and the solvents CD₃CN, CDCl₃ or D₆-DMSO, or with a Bruker Avance III HD 300 MHz Digital NMR with a 5 mm sample head.

The NMR data of selected examples are listed either in conventional form (6 values, multiplet splitting, number of hydrogen atoms) or as NMR peak lists.

### NMR Peak List Method

The ¹H NMR data of selected examples are stated in the form of ¹H NMR peak lists. For each signal peak, first the δ value in ppm and then the signal intensity in round brackets are listed. The δ value-signal intensity number pairs for different signal peaks are listed with separation from one another by semicolons.

The peak list for one example therefore has the form:

δ₁ (intensity₁); δ₂ (intensity₂); . . . ; δᵢ (intensityᵢ); . . . δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

Further details of ¹H NMR peak lists can be found in the Research Disclosure Database Number 564025.

### Preparation Example for Process C

### Preparation of tert-butyl 1-methyl-1H-imidazole-4-carboxylate

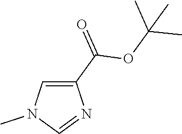

Under an atmosphere of argon, 1-methyl-1H-imidazolecarboxylic acid (100.0 g, 792 mmol) was dissolved in 1.1 l of dichloromethane, and 0.5 ml of DMF was added. At room temperature, oxalyl chloride (120.8 g, 951 mmol) was added and the mixture was stirred at room temperature for 12 h. The solvent was removed and the residue was dissolved in 795 ml of THF and cooled to −30° C. Slowly, lithium tert-butoxide solution (2.2 M in THF, 896.4 ml, 1972 mmol) was added dropwise to the mixture. After the addition, the reaction mixture was slowly warmed to room temperature. The mixture was stirred at room temperature for 12 h, and saturated NaHCO₃ solution was then added. The twophase mixture was stirred for 1 h, and most of the solvent was then removed on a rotary evaporator. The residue was taken up in ethyl acetate and the organic phase was washed with water. The aqueous phase was extracted with ethyl acetate. The combined organic extracts were washed with saturated NaCl solution and dried over Na₂SO₄ and the solvent was removed on a rotary evaporator. The crude product (86.7 g, 72%) was reacted in the next reaction without any further purification.

log P (acidic): 0.63; MH+: 183; 1H-NMR (400 MHz, D6-DMSO) δ ppm: 7.71 (s, 1H), 7.64 (s, 1H), 3.67 (s, 3H), 1.49 (s, 9H)

### Preparation of tert-butyl 2-bromo-1-methyl-1H-imidazole-4-carboxylate

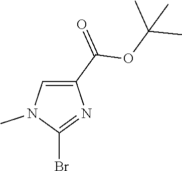

Under an atmosphere of argon, tert-butyl 1-methyl-1H-imidazole-4-carboxylate (64.0 g, 351 mmol, 1 eq.) and 1,2-dibromo-1,1,2,2-tetrachloroethane (114.4 g, 351 mmol, 1 eq.) were dissolved in 1200 ml of THF, and lithium tert-butoxide (84.3 g, 1.05 mmol, 3 eq.) was added a little at a time at −10-0° C. The reaction mixture was stirred for 12 h at room temperature. After removal of the solvent, the residue was taken up in ethyl acetate. The organic solution was washed with water, dried over Na₂SO₄ and freed of the solvent on a rotary evaporator. The crude product was purified chromatographically (mobile phase: cyclohexane/ethyl acetate). This gave 45.8 g (50%) of the desired product.

log P (acidic): 2.41; MH+: 261; 1H-NMR (400 MHz, D6-DMSO) δ ppm: 7.94 (s, 1H), 3.63 (s, 3H), 1.49 (s, 9H).

### Preparation of tert-butyl 2-(2,6-difluorophenyl)-1-methyl-1H-imidazole-4-carboxylate

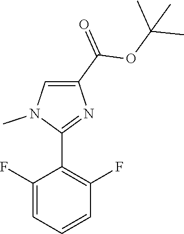 [SMILES: Cn1cc(C(=O)OC(C)(C)C)nc1-c1c(F)cccc1F]

A solution of potassium phosphate (152 mg, 0.71 mmol) in water (2.5 ml), X-phos Pd G2 catalyst (15 mg, 0.01 mmol) and 2-(2,6-difluorophenyl)-4,4,5,5-tetramethyl-1,3,2-dioxaborolane (276 mg, 1.14 mmol) were added to a solution of tert-butyl 2-bromo-1-methyl-1H-imidazole-4-carboxylate (100 mg, 0.38 mmol) in THF (15 ml). The reaction mixture was stirred at 80° C. for 6 h. A second portion of 2-(2,6-difluorophenyl)-4,4,5,5-tetramethyl-1,3,2-dioxaborolane (183 mg) was then added, and the mixture was stirred at 65° C. for a further 15 h. The reaction mixture was diluted with water (50 ml) and extracted with ethyl acetate (3×50 ml). The combined organic phases were dried over Na₂SO₄ and the solvent was removed under reduced pressure. Purification of the crude product by HPLC gave the desired product (58.1 mg, 52%).

log P (neutral): 2.3; MH+: 295.1; 1H-NMR (400 MHz, D6-DMSO) δ ppm: 8.00 (s, 1H), 7.67 (m, 1H), 7.32 (m, 2H), 3.54 (s, 3H), 1.51 (s, 9H).

### Preparation of 2-(2,6-difluorophenyl)-1-methyl-1H-imidazole-4-carboxylic acid

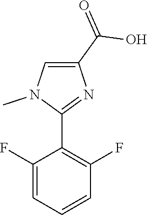 [SMILES: Cn1cc(C(=O)O)nc1-c1c(F)cccc1F]

tert-Butyl 2-(2,6-difluorophenyl)-1-methyl-1H-imidazole-4-carboxylate (58.0 mg, 0.19 mmol) was initially charged in dichloromethane (0.5 ml), and trifluoroacetic acid (0.11 ml) was added. The reaction mixture was stirred at room temperature for 15 h. The solvent was removed under reduced pressure and the residue was co-distilled with dichloromethane.

log P (neutral): 2.3; MH+: 239.1; 1H-NMR (400 MHz, D6-DMSO) δ ppm: 8.01 (s, 1H), 7.70 (m, 1H), 7.33 (m, 2H), 3.57 (s, 3H).

### Preparation Example Process D

### Preparation of ethyl 1,5-dimethyl-1H-imidazole-4-carboxylate

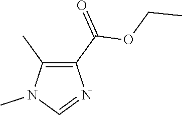

Ethyl 1,5-dimethyl-1H-imidazole-4-carboxylate 3-oxide (19.0 g, 103.1 mmol, 1 eq.; preparation analogously to European Journal of Organic Chemistry (2011), (13), 2542-2547,S2542/1-S2542/8. Helvetica Chimica Acta (2011), 94(10), 1764-1777. Helvetica Chimica Acta (2008), 91(10), 1916-1933) was dissolved in 93 ml of ethanol. Raney nickel (Ra-Ni 400, 4 g) was added and the mixture was stirred under a 5 bar hydrogen atmosphere at room temperature for 1 h. After removal of the catalyst by filtration over silica gel, the solution was concentrated and the crude product (17.8 g, 91%) was used for the next reaction without further purification.

log P (neutral): 0.85; MH+: 169;

### Preparation of Ethyl 2-Bromo-1,5-Dimethyl-1H-Imidazole-4-Carboxylate

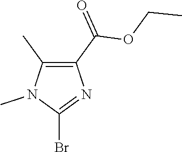

Ethyl 1,5-dimethyl-1H-imidazole-4-carboxylate (1.00 g; 5.49 mmol) and N-bromosuccinimide (0.98 g; 5.49 mmol) were dissolved in 100 ml of acetonitrile and stirred at room temperature for 4 h. The solvent was evaporated on a rotary evaporator and the residue was taken up in ethyl acetate. The organic phase was washed with NaHCO₃ solution and NaCl solution, dried over MgSO4 and freed of the solvent. The residue was purified chromatographically. This gave 616 mg (43%) of the desired product.

log P (neutral): 1.69; MH+: 263;

### Preparation of ethyl 2-(2,6-difluorophenyl)-1,5-dimethyl-1H-imidazole-4-carboxylate

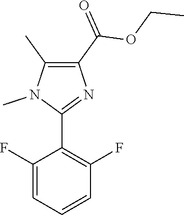 [SMILES: CCOC(=O)c1nc(-c2c(F)cccc2F)n(C)c1C]

Ethyl 2-(2,6-difluorophenyl)-1,5-dimethyl-1H-imidazole-4-carboxylate can be prepared analogously to tert-butyl 2-(2,6-difluorophenyl)-1-methyl-1H-imidazole-4-carboxylate.

log P (neutral): 2.0; MH+: 281.1; 1H-NMR (400 MHz, D6-DMSO) δ ppm: 7.68 (m, 1H), 7.32 (m, 2H), 4.22 (m, 2H), 3.41 (s, 3H), 2.49 (s, 3H), 1.28 (m, 3H).

### Preparation of 2-(2,6-difluorophenyl)-1,5-dimethyl-1H-imidazole-4-carboxylic acid

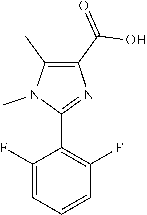 [SMILES: Cc1c(C(=O)O)nc(-c2c(F)cccc2F)n1C]

A solution of ethyl 2-(2,6-difluorophenyl)-1,5-dimethyl-1H-imidazole-4-carboxylate (779 mg, 2.77 mmol) and lithium hydroxide (79.9 mg, 3.33 mmol) in THF/water (3:1, 15 ml) was stirred at 60° C. for 15 h. The solvent was removed under reduced pressure.

log P (neutral): 2.0; MH+: 253.1; 1H-NMR (400 MHz, D6-DMSO) δ ppm: 8.32 (s, 1H), 7.63 (m, 1H), 7.29 (m, 2H), 3.45 (s, 3H), 2.46 (s, 3H).

### Preparation Example Process E

### Preparation of ethyl 2-(2,6-difluorophenyl)-5-ethyl-1H-imidazole-4-carboxylate

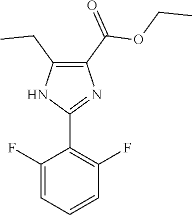 [SMILES: CCOC(=O)c1nc(-c2c(F)cccc2F)[nH]c1CC]

Ethyl (2Z)-2-(hydroxyimino)-3-oxopentanoate (4.33 g, 25 mmol, 1 eq.: preparation see Chemical & Pharmaceutical Bulletin, 2013, 61 (12), 1248) and 2,6-difluorobenzylamine (3.94 g, 27.5 mmol, 1.1 eq.) were dissolved in 30 ml of acetonitrile and heated at reflux for 12 h. The reaction mixture was concentrated and the residue was purified chromatographically. This gave 1.82 g (24%) of the desired product.

log P (acidic): 1.73; MH+: 281; 1H-NMR (400 MHz, D6-DMSO) δ ppm: 13.10 (2 s, 1H), 7.58 (m, 1H), 7.26 (m, 2H), 4.26 (m, 2H), 2.98 (m, 2H), 1.29 (m, 3H), 1.21 (m, 3H).

### Preparation of 2-(2,6-difluorophenyl)-5-ethyl-1H-imidazole-4-carboxylic acid

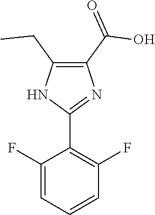 [SMILES: CCc1[nH]c(-c2c(F)cccc2F)nc1C(=O)O]

Analogously to the preparation of 2-(2,6-difluorophenyl)-1,5-dimethyl-1H-imidazole-4-carboxylic acid (xx), ethyl 2-(2,6-difluorophenyl)-5-ethyl-1H-imidazole-4-carboxylate (1.7 g, 6.07 mmol) was hydrolysed with aqueous sodium hydroxide solution in EtOH to give the free acid 2-(2,6-difluorophenyl)-5-ethyl-1H-imidazole-4-carboxylic acid (1.3 g, 68%).

log P (neutral): 0.45; MH+: 253;

### Preparation Example Process F

### Preparation of tert-butyl 1-methyl-1H-imidazole-5-carboxylate

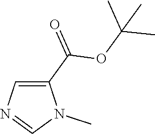

1-Methyl-1H-imidazole-5-carboxylic acid (18.0 g; 142.7 mmol; 1 eq.) was dissolved in 106 g of tertbutanol, and pyridine (79.0 g, 999 mmol, 7 eq.) and tosyl chloride (54.4 g, 285.4 mmol, 2 eq.) were added. The reaction mixture was stirred at room temperature for 12 h and then concentrated. The residue was taken up in dichloromethane, and NaHCO₃ solution was added until a pH of 8-9 had been reached. The aqueous phase was extracted repeatedly with dichloromethane and the combined organic extracts were dried over MgSO₄. After removal of the solvent the residue was purified chromatographically (mobile phase: cyclohexane/ethyl acetate). This gave 14.7 g (56%) of the desired product.

log P (neutral): 1.71; MH+: 183; 1H-NMR (400 MHz, D6-DMSO) δ ppm: 7.84 (s, 1H), 7.51 (s, 1H), 3.80 (s, 3H), 1.51 (s, 9H).

### Preparation of tert-butyl 2-bromo-1-methyl-1H-imidazole-5-carboxylate

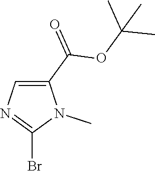

In a Schlenk flask which had been dried by heating und under an atmosphere of argon, tert-butyl 1-methyl-1H-imidazole-5-carboxylate (1.00 g, 5.38 mmol, 1 eq.) was dissolved in 30 ml of dry THF. The solution was cooled to −90° C. Over 20 min, 1.05 eq. of n-butyllithium (in hexane) were added dropwise. After 0.5 h of stirring, 1,2-dibromo-1,1,2,2-tetrachloroethane (1.75 g, 5.38 mmol, 1 eq.; dissolved in 15 ml THF) was added dropwise over a period of 20 min. The mixture was stirred at −80° C. for 2.5 h and then warmed to room temperature over 30 min. After addition of kieselguhr, the mixture was concentrated and repeatedly purified by chromatography (mobile phase: dichloromethane/methanol). This gave 438 mg (30%) of the desired product.

log P (neutral): 2.54; MH+: 263; 1H-NMR (400 MHz, D6-DMSO) δ ppm: 7.56 (m, 1H), 3.79 (m, 3H), 1.52 (s, 9H).

### Preparation of tert-butyl 2-(2,6-difluorophenyl)-1-methyl-1H-imidazole-5-carboxylate

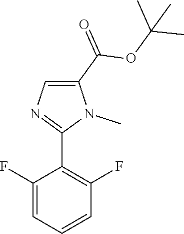 [SMILES: Cn1c(C(=O)OC(C)(C)C)cnc1-c1c(F)cccc1F]

tert-Butyl 2-(2,6-difluorophenyl)-1-methyl-1H-imidazole-5-carboxylate can be prepared analogously to tert-butyl 2-(2,6-difluorophenyl)-1-methyl-1H-imidazole-4-carboxylate.

log P (neutral): 3.0; MH+: 295.1; 1H-NMR (400 MHz, D6-DMSO) δ ppm: 7.74 (s, 1H), 7.71 (m, 1H), 7.34 (m, 2H), 3.66 (s, 3H), 1.55 (s, 9H).

### Preparation of 2-(2,6-difluorophenyl)-1-methyl-1H-imidazole-5-carboxylic acid

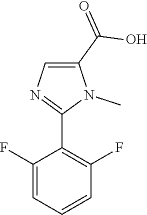 [SMILES: Cn1c(C(=O)O)cnc1-c1c(F)cccc1F]

2-(2,6-Difluorophenyl)-1-methyl-1H-imidazole-5-carboxylic acid can be prepared analogously to 2-(2,6-difluorophenyl)-1-methyl-1H-imidazole-4-carboxylic acid.

log P (acidic): 0.46; MH+: 239.1; 1H-NMR (400 MHz, D6-DMSO) δ ppm: 7.83 (s, 1H), 7.72 (m, 1H), 7.35 (m, 2H), 3.70 (s, 3H).

### Preparation of ethyl 1-(2-chlorophenyl)-5-hydroxy-1H-pyrazole-3-carboxylate

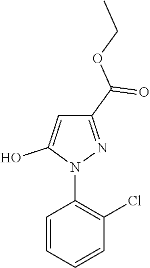 [SMILES: CCOC(=O)c1cc(O)n(-c2ccccc2Cl)n1]

16.9 g (94.3 mmol) of 2-chlorophenylhydrazine hydrochloride and 16.15 g (95 mmol) of diethyl acetylenedicarboxylate in about 250 ml of absolute ethanol and 26.5 g (191 mmol) of potassium carbonate were heated under reflux overnight. After evaporation of the solvent, water was added, the mixture was filtered off with suction through sand/Celite, aqueous citric acid was added to pH=4, the mixture 15 was stirred for 1 h and filtered off with suction and the residue was dried. Yield 20.68 g.

log P (acidic): 1.75; MH+: 267.1; 1H-NMR (400 MHz, D6-DMSO) δ ppm: 1.3 (t, 3H), 4.25 (q, 2H), 5.9 (s, 1H), 7.5-7.6 (m, 3H), 7.7 (m, 1H)

### Preparation of ethyl 1-(2-chlorophenyl)-5-methoxy-1H-pyrazole-3-carboxylate

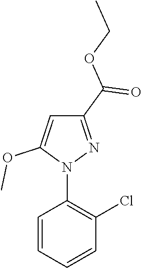 [SMILES: CCOC(=O)c1cc(OC)n(-c2ccccc2Cl)n1]

4.75 g (17.8 mmol) of ethyl 1-(2-chlorophenyl)-5-hydroxy-1H-pyrazole-3-carboxylate in about 200 ml of acetone were stirred successively in 2 batches with a total of 16.5 g (120.5 mmol) of methyl iodide and 22.5 g (162 mmol) of potassium carbonate. The mixture was filtered off with suction, the solvent was evaporated, the residue was dissolved in aqueous sodium chloride, ethyl acetate, Na-EDTA buffer at pH=9, the mixture was extracted twice with ethyl acetate, the combined organic phases were dried with sodium sulphate and the solvent was evaporated. The residue was purified by chromatography on silica gel (petroleum ether/acetone).

Yield 2.2 g.

log P (acidic): 2.61; MH+: 281.1; 1H-NMR (400 MHz, D6-DMSO) δ ppm: 1.3 (t, 3H), 3.9 (s, 3H), 4.3 (q, 2H), 6.3 (s, 1H), 7.5-7.6 (m, 3H), 7.7 (m, 1H)

### Preparation of 1-(2-chlorophenyl)-5-methoxy-1H-pyrazole-3-carboxylic acid

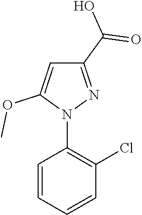 [SMILES: COc1cc(C(=O)O)nn1-c1ccccc1Cl]

Aqueous sodium hydroxide solution was added to 2.1 g (7.5 mmol) of ethyl 1-(2-chlorophenyl)-5-methoxy-1H-pyrazole-3-carboxylate in ethanol/water, and the mixture was stirred overnight at 70° C. The solvent was evaporated, water was added to the residue, dilute hydrochloric acid was added with ice bath cooling, the mixture was stirred overnight and filtered off with suction and the product was washed with water and dried.

Yield 2 g.

log P (acidic): 1.63; MH+: 253.0; 1H-NMR (400 MHz, D6-DMSO) δ ppm: 3.9 (s, 3H), 6.3 (s, 1H), 7.5-7.6 (m, 3H), 7.7 (m, 1H), 13 (s, 1H)

### Preparation of lithium (3-chloropyridin-2-yl)[tris(propan-2-olato)]borate(1-)

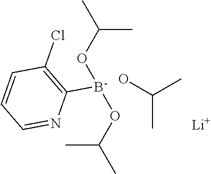

Under argon, 2-bromo-3-chloropyridine (50.0 g, 260 mmol) was dissolved in dry THF (200 ml) and dry toluene (800 ml) and the solution was cooled to −78° C. A 2.5 M solution of n-BuLi in hexane (100 ml, 250 mmol) was slowly added dropwise. The reaction mixture was stirred at −78° C. for 2 h, and triisopropyl borate (244 g, 1300 mmol) was then added. After a further 2 h at −78° C., the mixture was warmed to room temperature. Isopropanol (100 ml) was added and the resulting solution was concentrated at 40° C. Acetone (300 ml) was added to the residue and the mixture was stirred for 30 min. The precipitated solid was isolated by filtration, washed with acetone (2×100 ml) and dried.

1H-NMR (400 MHz, D20) 6 ppm: 8.30 (b-s, 1H), 7.80 (b-s, 1H), 7.30 (b-s, 1H), 3.85 (b-s, 3H), 1.00 (b-s, 18H).

### Preparation of tert-butyl 2-(3-chloropyridin-2-yl)-1-methyl-1H-imidazole-4-carboxylate

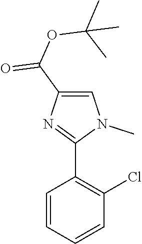 [SMILES: Cn1cc(C(=O)OC(C)(C)C)nc1-c1ccccc1Cl]

Under argon, lithium (3-chloropyridin-2-yl)[tris(propan-2-olato)]borate(1-) (487 mg, 1.50 mmol, 1 eq), tert-butyl 2-bromo-1-methyl-1H-imidazole-4-carboxylate (196 mg, 0.75 mmol, 0.5 eq), copper(I) chloride (74 mg, 0.75 mmol, 0.5 eq.), caesium carbonate (677 mg, 3 mmol, 2 eq.), 1,1′-bis(diphenylphosphino)ferrocene (42 mg, 0.07 mmol, 0.05 eq.) were dissolved in DMF (15 ml). The reaction solution was flushed with argon for a number of minutes, and palladium acetate (8 mg, 0.03 mmol, 0.025 eq) was then added. The reaction mixture was heated at 100° C. for 12 h. After cooling, the solvent was distilled off and the residue was taken up in ethyl acetate. The organic phase was washed first with a saturated ammonium chloride solution and then with water, dried over sodium sulphate and freed of the solvent on a rotary evaporator. The residue was purified chromatographically (SiO₂, mobile phase: cyclohexane/ethyl acetate 1:1).

MH+: 294; 1H-NMR (400 MHz, D6-DMSO) δ ppm: 8.68-8.67 (m, 1H); 8.16-8.14 (m, 1H), 7.95 (s, 1H), 7.61-7.58 (m, 1H), 3.64 (s, 3H), 1.52 (s, 9H).

### Preparation of 2-(3-chloropyridin-2-yl)-1-methyl-1H-imidazole-4-carboxylic acid

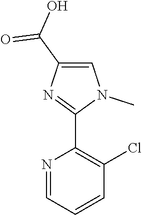 [SMILES: Cn1cc(C(=O)O)nc1-c1ncccc1Cl]

tert-Butyl 2-(3-chloropyridin-2-yl)-1-methyl-1H-imidazole-4-carboxylate (1.53 g, 5.07 mmol, 1 eq.) was dissolved in dichloromethane (15 ml), and trifluoroacetic acid (5.8 g, 50.7 mmol, 10 eq.) was added. The reaction solution was stirred at room temperature for 48 h, the solvent was then removed and the residue was purified chromatographically (SiO₂, mobile phase: ethyl acetate).

MH+: 238; 1H-NMR (400 MHz, D6-DMSO) δ ppm: DMSO 8.68-8.67 (m, 1H), 8.16-8.14 (m, 1H), 8.01 (s, 1H), 7.60-7.57 (m, 1H), 3.66 (s, 3H).

### Preparation of 2-(3-bromopyridin-2-yl)-1-methyl-1H-imidazole-4-carboxylic acid

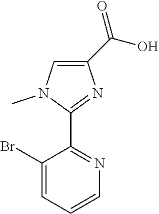 [SMILES: Cn1cc(C(=O)O)nc1-c1ncccc1Br]

Ethyl 2-(3-bromopyridin-2-yl)-1-methyl-1H-imidazole-4-carboxylate (2.11 g, 6.8 mmol, 1 eq.) and lithium hydroxide (0.244 g, 10.2 mmol, 1.5 eq.) were dissolved in a mixture of THF (31 ml) and water (31 ml). The reaction mixture was heated at reflux overnight. After concentration, dilute hydrochloric acid was added to pH 4 and the mixture was stirred at room temperature for 1 h. The resulting precipitate was isolated by filtration, washed with a little water and dried.

MH+: 284; 1H-NMR (400 MHz, D6-DMSO) δ ppm: 8.71-8.70 (m, 1H), 8.30-8.28 (m, 1H), 8.00 (s, 1H), 7.52-7.48 (m, 1H), 3.05 (s, 3H).

### Preparation of ethyl 2-(3-bromopyridin-2-yl)-1-methyl-1H-imidazole-4-carboxylate

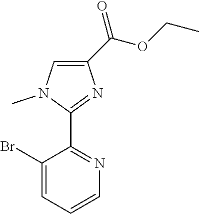 [SMILES: CCOC(=O)c1cn(C)c(-c2ncccc2Br)n1]

Under an atmosphere of argon, ethyl 2-(3-bromopyridin-2-yl)-1H-imidazole-4-carboxylate (3.25 g, 10.3 mmol, 1 eq.) was dissolved in dry THF (50 ml). The solution was cooled to −5° C. and sodium hydride (0.289 g, 11.4 mmol, 1.1 eq.) was added. After 30 min of stirring at −5° C., iodomethane (1.61 g, 11.4 mmol, 1.1 eq.) dissolved in THF (10 ml) was added dropwise and the mixture was stirred at 0° C. for a further 3 h. The mixture was warmed to room temperature and stirred for a further 12 h. After addition of dilute hydrochloric acid, the solution was concentrated. The residue was taken up in water, and triethylamine was added. The aqueous phase was extracted with ethyl acetate. The combined organic extracts were dried over sodium sulphate and freed of the solvent. The residue was purifled chromatographically (SiO₂, mobile phase cyclohexane ethyl acetate).

MH+: 312; 1H-NMR (400 MHz, D6-DMSO) δ ppm: 8.72-8.70 (m, 1H), 8.31-8.29 (m, 1H), 8.07 (s, 1H), 7.53-7.50 (m, 1H), 4.27-4.22 (q, 2H), 3.62 (s, 3H), 1.30-1.27 (t, 3H).

### Preparation of ethyl 2-(3-bromopyridin-2-yl)-1H-imidazole-4-carboxylate

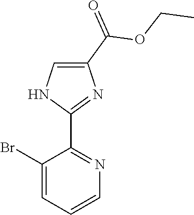 [SMILES: CCOC(=O)c1c[nH]c(-c2ncccc2Br)n1]

3-Bromo-N′-hydroxypyridine-2-carboximidamide (9.81 g, 45.4 mmol, 1 eq.) and ethyl propionate (4.46 g, 45.4 mmol, 1 eq.) were dissolved in ethanol (44 ml) and heated to reflux for 12 h. Ethanol was distilled off, toluene was added to the residue and the solvent was distilled off again. The residue was taken up in diphenyl ether (27 ml) and heated at 195° C. for 2.5 h. After cooling to 70° C., hexane (200 ml) was added and the mixture was stirred for 3 h. The solution was decanted off and the solid obtained was purified chromatographically (reversed phase; solvent: acetonitrile, water). The supernatant hexane phase was concentrated and likewise purified chromatographically (SiO2, mobile phase: cyclohexane ethyl acetate; then reversed phase, mobile phase acetonitrile/water).

MH+: 298; 1H-NMR (400 MHz, D6-DMSO) δ ppm: 8.68-8.66 (m, 1H), 8.26-8.24 (m, 1H), 7.93-7.92 (m, 1H), 7.42-7.39 (m, 1H), 4.29-4.24 (q, 2H), 1.31-1.27 (t, 3H).

### Preparation of 3-bromo-N′-hydroxypyridine-2-carboximidamide

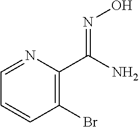 [SMILES: N/C(=N\O)c1ncccc1Br]

3-Bromopyridine-2-carbonitrile (13.2 g, 72.1 mmol, 1 eq.), hydroxylammonium chloride (11.0 g, 158 mmol, 2.2 eq.) and potassium carbonate (21.9 g, 158 mmol, 2.2 eq.) were dissolved in ethanol (100 ml) and heated at reflux for 12 h. The reaction mixture was concentrated and dissolved in water. The pH was adjusted to 4 by addition of dilute hydrochloric acid and the mixture was stirred for 1 h. The resulting solid was isolated by filtration, washed and dried.

MH+: 217; 1H-NMR (400 MHz, D6-DMSO) δ ppm: 9.72 (s, 1H), 8.58-8.57 (s, 1H), 8.14-8.12 (s, 1H), 7.38-7.35 (m, 1H), 5.81 (s, 2H).

### Preparation Example for the Preparation of a Sulphonamide

### Preparation of 5-chloro-2-(2-chlorophenyl)-1-methyl-N-[(2-methylphenyl)sulphonyl]-1 Himidazole-4-carboxamide

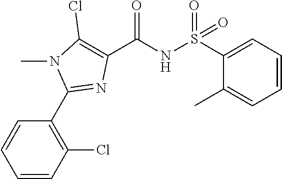 [SMILES: Cc1ccccc1S(=O)(=O)NC(=O)c1nc(-c2ccccc2Cl)n(C)c1Cl]

5-Chloro-2-(2-chlorophenyl)-1-methyl-1H-imidazole-4-carboxylic acid (163 mg; 0.60 mmol; 1 eq.) was dissolved in 10 ml of dichloromethane. After addition of 1-ethyl-3-(3-dimethylaminopropyl)carbodiimide hydrochloride (345 mg, 1.8 mmol, 3 eq.) and DMAP (220 mg, 1.8 mmol, 3 eq.), the mixture was stirred at room temperature for 1 h. 2-Methylbenzenesulphonamide (103 mg; 0.6 mmol, 1 eq.) was then added, and the mixture was subsequently stirred at room temperature for 48 h. The solvent was removed on a rotary evaporator and the residue was purified chromatographically (reversed phase; mobile phase: acetonitrile, H₂O). This gave 134 mg (52%) of the desired product.

log P (acidic): 3.17; MH+: 424; 1H-NMR (400 MHz, D6-DMSO) δ ppm: 8.04 (d, 1H); 7.65-7.52 (m, 5H), 7.46-7.38 (m, 2H), 3.41 (s, 3H); 2.61 (s, 3H).

Table 1 lists further compounds of the formula (I) which were prepared analogously to the examples given above. The synthesis of the acid precursors was either as described above, or the acids were commercially available.

TABLE 1

| | Structure | NMR peak list |
|---|---|---|
| 1 | 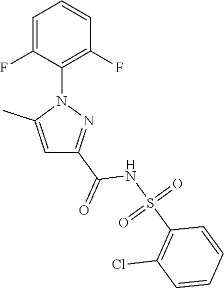 [SMILES: Cc1cc(C(=O)NS(=O)(=O)c2ccccc2Cl)nn1-c1c(F)cccc1F] | Example 1: ¹H-NMR (300 MHz, CD₃OH): 2.4 (s, 3H), 6.75 (s, 1H), 7.25 (m, 2H), 7.5 (m, 3H), 7.6 (m, 1H), 8.25 (m, 1H) |
| 2 | 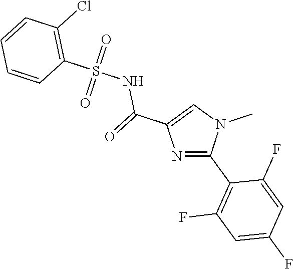 [SMILES: Cn1cc(C(=O)NS(=O)(=O)c2ccccc2Cl)nc1-c1c(F)cc(F)cc1F] | Example 2: ¹H-NMR(400.0 MHz, d₆-DMSO): d = 8.236(4.4); 8.159(2.0); 8.155(2.3); 8.139(2.3); 8.136(2.3); 7.720(0.5); 7.716(0.6); 7.700(1.6); 7.682 (1.9); 7.679(1.9); 7.665(2.7); 7.662(3.4); 7.646(1.4); 7.642(1.1); 7.626(1.5); 7.622(1.3); 7.606(2.0); 7.589(1.0); 7.585(0.9); 7.487(2.3); 7.467(3.4); 7.464 (3.5); 7.444(2.3); 3.580(16.0); 3.511(0.4); 2.675(0.4); 2.671(0.5); 2.666(0.4); 2.524(1.7); 2.510(29.2); 2.506(56.9); 2.502(74.4); 2.497(55.5); 2.493(28.3); 2.333(0.4); 2.328(0.5); 2.324(0.4); 2.073(6.5); 0.000(4.8) |
| 3 | 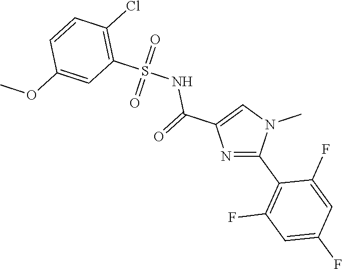 [SMILES: COc1ccc(Cl)c(S(=O)(=O)NC(=O)c2cn(C)c(-c3c(F)cc(F)cc3F)n2)c1] | Example 1: ¹H-NMR: (400.0 MHz, d₆-DMSO): d = 8.235(3.0); 7.604(2.8); 7.596(2.9); 7.569(2.3); 7.547(2.7); 7.489(1.5); 7.469(2.3); 7.466(2.3); 7.446 (1.5); 7.291(1.3); 7.283(1.3); 7.269(1.2); 7.261(1.1); 3.853(16.0); 3.583 (10.3); 2.675(0.4); 2.671(0.5); 2.666(0.4); 2.524(1.5); 2.510(25.2); 2.506 (49.7); 2.501(65.2); 2.497(48.3); 2.493(24.1); 2.328(0.4); 0.000(4.6) |
| 4 | 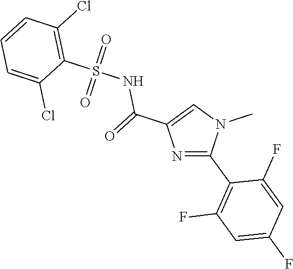 [SMILES: Cn1cc(C(=O)NS(=O)(=O)c2c(Cl)cccc2Cl)nc1-c1c(F)cc(F)cc1F] | Example 4: ¹H-NMR(400.0 MHz, d₆-DMSO): d = 8.222(1.2); 7.655(0.9); 7.651(1.1); 7.633(2.5); 7.596(0.9); 7.579(0.6); 7.573(0.5); 7.556(0.3); 7.495(0.9); 7.475(1.3); 7.472(1.4); 7.452(0.9); 3.589(5.8); 3.556(0.3); 2.675(0.3); 2.670(0.4); 2.666(0.3); 2.506(54.3); 2.501(70.4); 2.497(54.4); 2.332(0.4); 2.328(0.5); 2.324(0.4); 2.073(16.0); 0.000(4.1) |
| 5 | 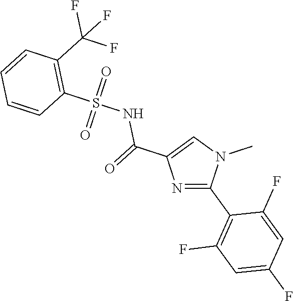 [SMILES: Cn1cc(C(=O)NS(=O)(=O)c2ccccc2C(F)(F)F)nc1-c1c(F)cc(F)cc1F] | Example 5: ¹H-NMR(400.0 MHz, d₆-DMSO): d = 8.346(2.0); 8.327(2.1); 8.226(6.1); 8.006(1.5); 7.990(2.1); 7.987(2.3); 7.966(0.8); 7.951(2.0); 7.948 (1.7); 7.932(1.7); 7.928(1.4); 7.919(1.7); 7.900(1.7); 7.882(0.6); 7.495(2.3); 7.485(0.5); 7.475(3.3); 7.472(3.4); 7.452(2.3); 3.585(16.0); 3.535(0.4); 2.675 (0.4); 2.671(0.6); 2.666(0.4); 2.524(1.9); 2.510(31.8); 2.506(64.2); 2.502 (86.0); 2.497(64.5); 2.493(33.3); 2.333(0.5); 2.328(0.6); 2.324(0.5); 2.073 (1.2); 0.146(0.6); 0.008(4.8); 0.000(126.6); −0.009(6.4); −0.030(0.5); −0.150 (0.6) |
| 6 | 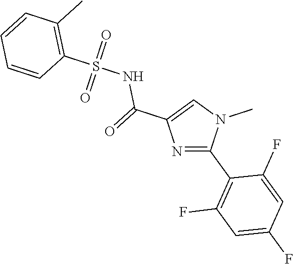 [SMILES: Cc1ccccc1S(=O)(=O)NC(=O)c1cn(C)c(-c2c(F)cc(F)cc2F)n1] | Example 6: ¹H-NMR(400.0 MHz, d₆-DMSO): d = 8.174(6.5); 8.028(2.1); 8.026(2.2); 8.009(2.3); 8.006(2.3); 7.588(0.9); 7.585(0.9); 7.569(2.2); 7.566 (2.2); 7.550(1.5); 7.547(1.4); 7.498(0.3); 7.485(2.4); 7.476(0.7); 7.465(3.6); 7.462(3.7); 7.452(2.0); 7.442(2.7); 7.435(2.8); 7.414(1.1); 7.397(2.4); 7.378 (2.0); 3.568(15.7); 3.480(0.3); 3.388(0.4); 3.354(0.4); 3.344(0.4); 3.327(0.4); 3.305(0.4); 3.294(0.4); 3.289(0.4); 3.277(0.3); 2.675(0.3); 2.671(0.4); 2.666 (0.3); 2.603(16.0); 2.510(23.5); 2.506(45.5); 2.502(59.6); 2.497(45.2); 2.493 (24.1); 2.328(0.4); 2.073(5.1); 0.146(0.4); 0.008(4.1); 0.000(86.6); −0.008 (5.2); −0.030(0.7); −0.150(0.4) |
| 7 | 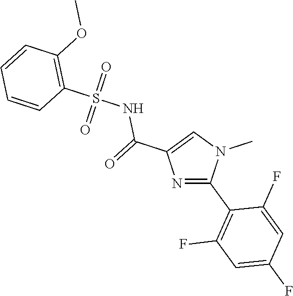 [SMILES: COc1ccccc1S(=O)(=O)NC(=O)c1cn(C)c(-c2c(F)cc(F)cc2F)n1] | Example 7: ¹H-NMR(400.0 MHz, d₆-DMSO): d = 8.214(3.7); 7.901(1.6); 7.897(1.7); 7.881(1.8); 7.877(1.8); 7.685(0.7); 7.680(0.7); 7.663(1.3); 7.645 (0.9); 7.641(0.8); 7.490(1.7); 7.481(0.4); 7.470(2.5); 7.467(2.5); 7.447(1.7); 7.235(2.0); 7.215(1.8); 7.163(1.1); 7.161(1.1); 7.143(1.9); 7.125(1.0); 3.843 (16.0); 3.576(11.4); 3.318(2.6); 2.670(0.4); 2.524(1.6); 2.510(24.1); 2.506 (47.4); 2.501(62.1); 2.497(46.1); 2.492(23.4); 2.328(0.4); 0.146(0.4); 0.030 (0.3); 0.008(3.9); 0.000(90.7); −0.008(4.5); −0.021(0.5); −0.024(0.4); −0.150 (0.4) |
| 8 | 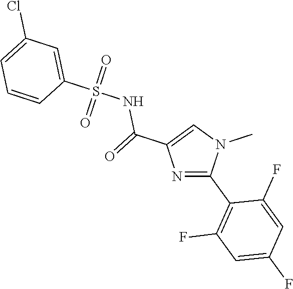 [SMILES: Cn1cc(C(=O)NS(=O)(=O)c2cccc(Cl)c2)nc1-c1c(F)cc(F)cc1F] | Example 8: ¹H-NMR(400.0 MHz, d₆-DMSO): d = 8.184(6.9); 7.994(1.9); 7.990(3.6); 7.985(2.4); 7.954(1.9); 7.934(2.2); 7.810(1.4); 7.807(1.4); 7.805 (1.3); 7.792(1.7); 7.790(1.9); 7.787(1.9); 7.784(1.7); 7.691(2.5); 7.671(3.8); 7.651(1.7); 7.509(0.3); 7.495(2.4); 7.485(0.6); 7.475(3.4); 7.472(3.5); 7.452 (2.4); 3.606(0.4); 3.571(16.0); 3.548(0.7); 3.503(0.6); 3.478(0.4); 3.415(0.4); 3.391(0.5); 3.353(0.4); 3.318(0.4); 3.309(0.4); 3.293(0.4); 2.891(0.6); 2.732 (0.5); 2.675(0.6); 2.670(0.8); 2.666(0.6); 2.645(1.3); 2.524(2.5); 2.510(41.2); 2.506(82.4); 2.502(109.7); 2.497(82.3); 2.493(42.7); 2.333(0.6); 2.328(0.8); 2.324(0.6); 2.073(1.1); 0.146(0.7); 0.031(0.4); 0.008(5.9); 0.000(151.9); −0.008(8.2); −0.150(0.8) |
| 9 | 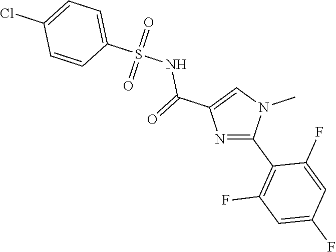 [SMILES: Cn1cc(C(=O)NS(=O)(=O)c2ccc(Cl)cc2)nc1-c1c(F)cc(F)cc1F] | Example 9: ¹H-NMR(400.0 MHz, d₆-DMSO): d = 8.170(6.6); 8.000(5.3); 7.979(6.1); 7.722(6.1); 7.701(5.3); 7.490(2.2); 7.469(3.8); 7.447(2.2); 3.566 (16.0); 3.540(0.6); 3.488(0.6); 2.670(0.5); 2.505(55.9); 2.501(67.6); 2.328 (0.5); 2.074(1.4); 2.072(1.4); 0.145(0.4); 0.000(73.1); −0.002(73.3); −0.151 (0.4) |
| 10 | 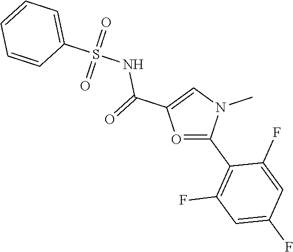 | Example 10: ¹H-NMR(400.0 MHz, d₆-DMSO): d = 8.160(7.6); 8.004(4.2); 7.985(4.9); 7.982(3.6); 7.724(0.8); 7.711(0.7); 7.706(2.5); 7.701(0.9); 7.687 (1.9); 7.643(3.6); 7.624(4.9); 7.605(1.9); 7.500(0.4); 7.486(2.4); 7.467(3.5); 7.463(3.5); 7.444(2.4); 3.562(16.0); 3.533(0.4); 3.474(0.5); 3.419(0.3); 3.382 (0.4); 3.343(0.4); 3.320(0.4); 3.299(0.4); 3.271(0.3); 2.676(0.3); 2.671(0.4); 2.666(0.3); 2.510(23.7); 2.506(45.9); 2.502(60.1); 2.497(44.6); 2.493(22.9); 2.328(0.4); 2.073(3.0); 0.146(0.4); 0.008(3.7); 0.000(81.8); −0.008(4.2); −0.150(0.4) |
| 11 | 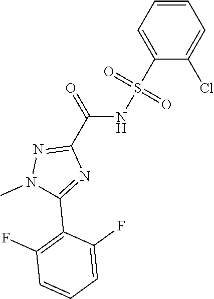 [SMILES: Cn1nc(C(=O)NS(=O)(=O)c2ccccc2Cl)nc1-c1c(F)cccc1F] | Example 11: ¹H-NMR(400.0 MHz, d₆-DMSO): d = 8.187(2.1); 8.184(2.2); 8.168(2.3); 8.164(2.3); 7.829(0.4); 7.813(0.9); 7.808(0.9); 7.791(1.8); 7.774 (0.9); 7.770(1.1); 7.753(0.5); 7.748(0.6); 7.744(0.6); 7.728(1.7); 7.724(1.6); 7.710(2.0); 7.707(2.0); 7.693(2.8); 7.689(3.6); 7.673(1.4); 7.669(1.1); 7.648 (1.5); 7.644(1.4); 7.628(2.0); 7.611(1.0); 7.607(0.9); 7.427(3.1); 7.406(5.3); 7.385(2.6); 3.879(16.0); 2.671(0.4); 2.524(0.9); 2.506(44.0); 2.502(58.1); 2.498(43.1); 2.329(0.4); 0.008(1.0); 0.000(30.6); −0.008(1.4) |
| 12 | 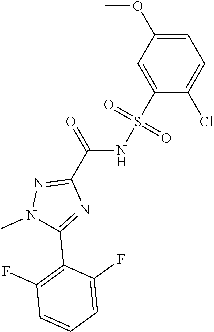 [SMILES: COc1ccc(Cl)c(S(=O)(=O)NC(=O)c2nc(-c3c(F)cccc3F)n(C)n2)c1] | Example 12: ¹H-NMR(400.0 MHz, d₆-DMSO): d = 7.812(0.6); 7.808(0.7); 7.791(1.2); 7.774(0.7); 7.770(0.7); 7.753(0.3); 7.625(2.7); 7.617(2.9); 7.595 (2.6); 7.573(3.0); 7.427(2.2); 7.406(3.8); 7.385(1.9); 7.317(1.5); 7.309(1.5); 7.295(1.3); 7.287(1.3); 3.878(11.4); 3.861(16.0); 2.671(0.3); 2.506(37.9); 2.502(50.0); 2.497(37.9); 2.329(0.3); 2.074(0.4); 0.007(0.9); 0.000(22.7); −0.008(1.2) |
| 13 | 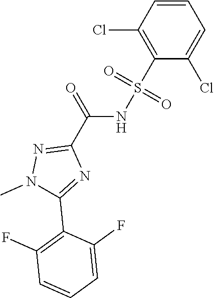 [SMILES: Cn1nc(C(=O)NS(=O)(=O)c2c(Cl)cccc2Cl)nc1-c1c(F)cccc1F] | Example 13: ¹H-NMR(400.0 MHz, d₆-DMSO): d = 7.828(0.4); 7.812(1.0); 7.807(1.0); 7.795(0.7); 7.790(1.8); 7.774(0.9); 7.769(1.1); 7.752(0.5); 7.688 (2.8); 7.683(3.7); 7.665(9.0); 7.632(3.9); 7.615(2.3); 7.609(1.7); 7.592(1.2); 7.425(3.2); 7.404(5.4); 7.383(2.6); 3.879(16.0); 2.524(0.5); 2.511(16.5); 2.506(35.5); 2.502(48.8); 2.497(37.1); 2.493(19.4); 2.328(0.4); 0.000(3.5) |
| 14 | 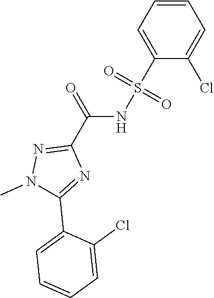 [SMILES: Cn1nc(C(=O)NS(=O)(=O)c2ccccc2Cl)nc1-c1ccccc1Cl] | Example 14: ¹H-NMR(400.0 MHz, d₆-DMSO): d = 8.186(1.5); 8.182(1.7); 8.166(1.6); 8.163(1.7); 7.745(0.4); 7.741(0.5); 7.726(2.4); 7.722(1.4); 7.708 (3.6); 7.704(3.7); 7.691(2.0); 7.687(2.7); 7.682(1.1); 7.677(1.5); 7.671(2.4); 7.666(2.1); 7.660(2.2); 7.652(2.3); 7.647(3.1); 7.643(2.3); 7.627(1.5); 7.610 (0.7); 7.606(0.7); 7.579(1.2); 7.576(1.3); 7.558(1.7); 7.542(0.6); 7.539(0.7); 3.791(16.0); 2.670(0.4); 2.523(1.0); 2.510(21.5); 2.505(44.4); 2.501(59.9); 2.496(45.2); 2.492(23.4); 2.328(0.4); 0.000(4.1) |
| 15 | 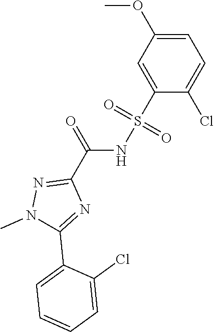 [SMILES: COc1ccc(Cl)c(S(=O)(=O)NC(=O)c2nc(-c3ccccc3Cl)n(C)n2)c1] | Example 15: ¹H-NMR(400.0 MHz, d₆-DMSO): d = 7.728(1.1); 7.717(0.4); 7.710(2.1); 7.706(2.1); 7.683(0.8); 7.678(1.5); 7.675(1.6); 7.670(1.3); 7.661 (2.1); 7.655(2.4); 7.651(1.8); 7.644(1.2); 7.639(0.5); 7.625(2.8); 7.618(2.9); 7.597(2.6); 7.580(1.4); 7.575(3.5); 7.559(1.7); 7.543(0.6); 7.540(0.6); 7.317 (1.5); 7.309(1.5); 7.295(1.3); 7.287(1.3); 3.862(16.0); 3.793(14.7); 2.524(0.7); 2.510(17.2); 2.506(34.6); 2.502(45.7); 2.497(34.5); 2.074(0.8); 0.000(0.9) |
| 16 | 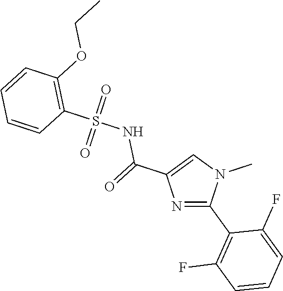 [SMILES: CCOc1ccccc1S(=O)(=O)NC(=O)c1cn(C)c(-c2c(F)cccc2F)n1] | Example 16: ¹H-NMR(400.0 MHz, d₆-DMSO): d = 8.199(4.1); 7.900(2.0); 7.896(2.2); 7.880(2.1); 7.877(2.2); 7.750(0.4); 7.733(0.9); 7.728(1.0); 7.712 (1.7); 7.694(1.0); 7.691(1.1); 7.674(0.5); 7.658(1.0); 7.640(1.9); 7.622(1.1); 7.619(1.0); 7.364(2.9); 7.343(4.7); 7.323(2.5); 7.217(2.6); 7.196(2.4); 7.147 (1.5); 7.128(2.7); 7.109(1.3); 4.150(1.5); 4.133(4.6); 4.115(4.6); 4.098(1.5); 3.569(16.0); 3.390(0.3); 3.318(4.4); 2.671(0.6); 2.506(78.0); 2.501(99.9); 2.497(77.0); 2.328(0.6); 2.073(1.8); 1.265(5.0); 1.248(10.3); 1.230(4.9); 0.000(7.0) |
| 17 | 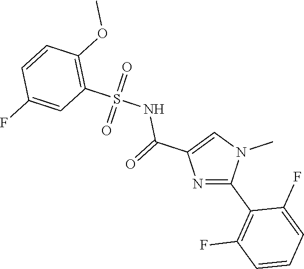 [SMILES: COc1ccc(F)cc1S(=O)(=O)NC(=O)c1cn(C)c(-c2c(F)cccc2F)n1] | Example 17: ¹H-NMR(400.0 MHz, d₆-DMSO): d = 8.227(2.3); 7.726(0.8); 7.722(0.7); 7.705(1.4); 7.688(0.8); 7.684(0.9); 7.666(1.6); 7.657(1.6); 7.645 (1.4); 7.637(1.5); 7.587(0.5); 7.580(0.5); 7.565(1.0); 7.558(0.9); 7.545(0.7); 7.537(0.5); 7.356(2.4); 7.336(3.8); 7.315(2.1); 7.289(1.2); 7.279(1.3); 7.266 (1.1); 7.256(1.0); 3.839(16.0); 3.572(12.6); 3.392(0.6); 3.330(0.9); 2.675(0.5); 2.671(0.6); 2.666(0.5); 2.506(74.5); 2.501(94.7); 2.497(70.6); 2.328(0.6); 0.008(0.4); 0.000(7.6) |
| 18 | 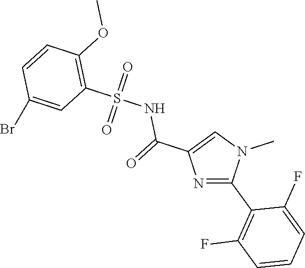 [SMILES: COc1ccc(Br)cc1S(=O)(=O)NC(=O)c1cn(C)c(-c2c(F)cccc2F)n1] | Example 18: ¹H-NMR(400.0 MHz, d₆-DMSO): d = 8.249(3.4); 7.745(0.4); 7.732(2.8); 7.710(3.7); 7.689(1.0); 7.671(0.4); 7.651(2.8); 7.643(3.0); 7.356(2.3); 7.336(4.0); 7.316(2.0); 7.208(1.4); 7.201(1.4); 7.186(1.3); 7.178(1.3); 3.849(16.0); 3.665(0.4); 3.618(0.3); 3.580(13.4); 3.523(0.4); 3.516(0.4); 3.487(0.4); 3.463(0.4); 3.400(0.5); 3.369(0.4); 2.671(0.7); 2.501(126.7); 2.328(0.9); 2.073(1.3); 0.000(4.7) |
| 19 | 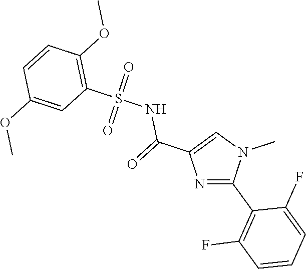 [SMILES: COc1ccc(OC)c(S(=O)(=O)NC(=O)c2cn(C)c(-c3c(F)cccc3F)n2)c1] | Example 19: ¹H-NMR(400.0 MHz, d₆-DMSO): d = 8.218(2.3); 7.726(0.6); 7.705(0.9); 7.687(0.6); 7.381(1.9); 7.374(2.1); 7.357(1.5); 7.337(2.6); 7.317 (1.3); 7.267(0.7); 7.260(0.6); 7.245(1.2); 7.237(1.1); 7.189(2.0); 7.166(1.1); 3.787(16.0); 3.572(8.5); 3.390(0.4); 3.318(1.3); 2.671(0.3); 2.501(51.7); 2.328(0.3); 0.000(1.9) |
| 20 | 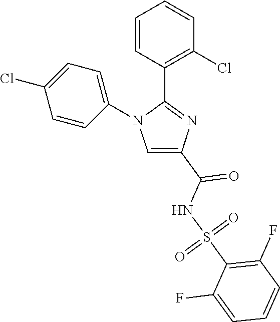 [SMILES: O=C(NS(=O)(=O)c1c(F)cccc1F)c1cn(-c2ccc(Cl)cc2)c(-c2ccccc2Cl)n1] | Example 20: ¹H-NMR(400.0 MHz, d₆-DMSO): d = 8.463(6.5); 7.810(0.4); 7.794(1.0); 7.788(1.0); 7.773(1.7); 7.758(1.0); 7.752(1.1); 7.737(0.5); 7.682 (2.6); 7.667(2.4); 7.663(2.8); 7.532(0.7); 7.528(0.8); 7.520(0.5); 7.515(0.9); 7.508(2.8); 7.498(7.9); 7.490(4.0); 7.481(3.1); 7.476(13.1); 7.456(2.6); 7.453 (3.7); 7.449(2.9); 7.435(1.2); 7.431(1.1); 7.350(3.0); 7.327(4.7); 7.301(10.6); 7.296(3.0); 7.284(2.4); 7.279(7.5); 7.272(0.7); 3.857(0.4); 3.482(16.0); 2.996 (0.5); 2.712(0.9); 2.676(0.7); 2.672(1.0); 2.667(0.8); 2.663(0.4); 2.562(0.6); 2.542(209.4); 2.525(2.5); 2.520(3.4); 2.511(51.7); 2.507(106.1); 2.503 (141.6); 2.498(105.3); 2.494(52.9); 2.368(1.0); 2.334(0.7); 2.329(1.0); 2.325(0.8); 1.235(0.7); 0.000(1.3) |
| 21 | 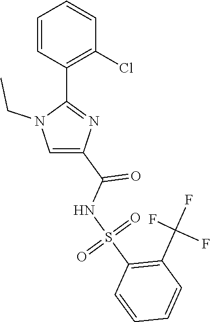 [SMILES: CCn1cc(C(=O)NS(=O)(=O)c2ccccc2C(F)(F)F)nc1-c1ccccc1Cl] | Example 21: ¹H-NMR(400.0 MHz, d₆-DMSO): d = 8.349(3.2); 8.329(3.5); 8.261(10.8); 7.973(2.7); 7.954(3.8); 7.936(1.4); 7.919(3.1); 7.900(2.3); 7.876 (2.5); 7.857(2.8); 7.839(1.0); 7.695(2.2); 7.677(5.3); 7.674(3.7); 7.651(1.9); 7.646(2.4); 7.628(6.7); 7.610(7.1); 7.551(2.6); 7.548(2.8); 7.532(3.7); 7.514 (1.3); 7.511(1.3); 3.848(2.0); 3.830(6.3); 3.812(6.4); 3.794(2.1); 2.714(0.4); 2.544(80.6); 2.523(0.8); 2.509(16.9); 2.505(22.6); 2.500(17.8); 2.370(0.4); 1.253(7.5); 1.235(16.0); 1.217(7.4); 0.000(0.6) |
| 22 | 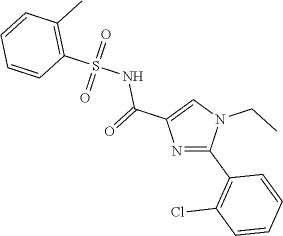 [SMILES: CCn1cc(C(=O)NS(=O)(=O)c2ccccc2C)nc1-c1ccccc1Cl] | Example 22: ¹H-NMR(400.0 MHz, d₆-DMSO): d = 8.201(7.8); 8.033(2.0); 8.031(2.2); 8.014(2.2); 8.011(2.3); 7.671(1.5); 7.669(1.6); 7.651(3.0); 7.649 (3.1); 7.620(1.2); 7.615(1.4); 7.602(1.6); 7.598(2.1); 7.581(2.5); 7.577(2.5); 7.566(2.3); 7.561(4.3); 7.556(2.3); 7.548(1.5); 7.545(1.5); 7.526(1.9); 7.524 (2.0); 7.507(2.1); 7.490(0.7); 7.487(0.8); 7.452(1.3); 7.433(2.1); 7.414(1.0); 7.399(2.3); 7.380(1.9); 3.812(1.5); 3.794(4.5); 3.776(4.6); 3.757(1.6); 2.611 (16.0); 2.542(57.0); 2.525(0.5); 2.520(0.6); 2.511(10.1); 2.507(21.2); 2.503 (28.6); 2.498(21.6); 2.494(11.2); 2.368(0.4); 1.231(5.2); 1.213(11.1); 1.194 (5.1); 0.000(0.9) |
| 23 | 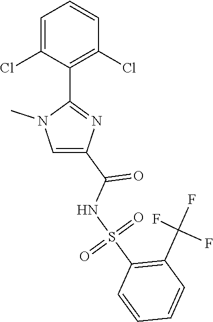 [SMILES: Cn1cc(C(=O)NS(=O)(=O)c2ccccc2C(F)(F)F)nc1-c1c(Cl)cccc1Cl] | Example 23: ¹H-NMR(400.0 MHz, d₆-DMSO): d = 8.364(1.7); 8.344(1.9); 8.238(6.3); 8.008(1.4); 7.989(2.1); 7.977(0.8); 7.961(1.7); 7.941(1.3); 7.920 (1.4); 7.902(1.5); 7.883(0.5); 7.711(2.0); 7.704(2.8); 7.688(7.8); 7.665(3.7); 7.650(2.0); 7.641(1.2); 7.626(0.9); 3.494(16.0); 2.715(0.3); 2.546(52.0); 2.515(4.0); 2.510(8.2); 2.506(11.0); 2.501(8.1); 2.497(4.1); 0.000(0.3) |
| 24 | 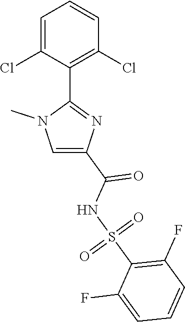 [SMILES: Cn1cc(C(=O)NS(=O)(=O)c2c(F)cccc2F)nc1-c1c(Cl)cccc1Cl] | Example 24: ¹H-NMR(400.0 MHz, d₆-DMSO): d = 8.243(6.8); 7.780(0.4); 7.765(0.8); 7.759(0.8); 7.744(1.5); 7.728(0.9); 7.720(2.8); 7.713(3.5); 7.696 (9.1); 7.675(4.3); 7.660(2.3); 7.651(1.4); 7.636(1.0); 7.321(2.5); 7.298(3.9); 7.275(2.2); 3.507(16.0); 2.543(11.0); 2.508(5.3); 2.504(11.6); 2.499(12.0); 2.495(9.1); 2.369(0.5); 2.330(0.4) |
| 25 | 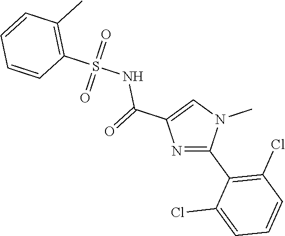 [SMILES: Cc1ccccc1S(=O)(=O)NC(=O)c1cn(C)c(-c2c(Cl)cccc2Cl)n1] | Example 25: ¹H-NMR(400.0 MHz, d₆-DMSO): d = 8.176(6.4); 8.037(1.9); 8.019(1.9); 8.017(2.0); 7.699(2.1); 7.693(3.0); 7.677(7.4); 7.676(7.4); 7.651 (3.6); 7.636(2.0); 7.627(1.3); 7.612(0.9); 7.593(0.7); 7.590(0.8); 7.574(1.8); 7.571(1.9); 7.556(1.2); 7.553(1.2); 7.461(1.2); 7.442(1.8); 7.423(0.8); 7.405 (2.0); 7.386(1.6); 3.468(16.0); 2.608(13.3); 2.543(35.0); 2.512(5.7); 2.508 (12.3); 2.503(16.8); 2.499(13.1); 2.495(7.1); 0.000(0.4) |
| 26 | 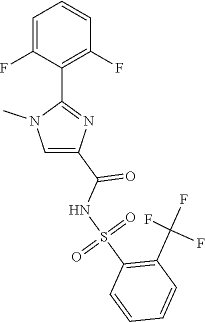 [SMILES: Cn1cc(C(=O)NS(=O)(=O)c2ccccc2C(F)(F)F)nc1-c1c(F)cccc1F] | Example 26: ¹H-NMR(400.0 MHz, d₆-DMSO): d = 8.355(2.1); 8.336(2.3); 8.242(7.1); 8.012(1.7); 7.993(2.5); 7.972(0.8); 7.956(2.1); 7.937(1.7); 7.934 (1.4); 7.922(1.8); 7.904(1.8); 7.885(0.6); 7.756(0.4); 7.739(1.0); 7.735(0.9); 7.718(1.8); 7.701(1.0); 7.697(1.1); 7.680(0.5); 7.366(3.1); 7.346(4.8); 7.326 (2.6); 4.155(0.5); 4.098(0.5); 4.041(0.5); 3.999(0.5); 3.587(16.0); 2.546(52.5); 2.529(0.5); 2.524(0.4); 2.511(11.4); 2.507(15.0); 2.502(11.4) |
| 27 | 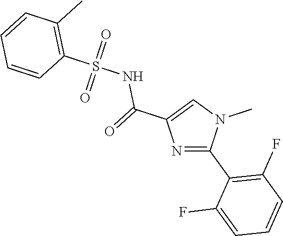 [SMILES: Cc1ccccc1S(=O)(=O)NC(=O)c1cn(C)c(-c2c(F)cccc2F)n1] | Example 27: ¹H-NMR(400.0 MHz, d₆-DMSO): d = 8.176(5.7); 8.028(2.2); 8.008(2.3); 7.740(0.4); 7.724(0.9); 7.719(0.9); 7.702(1.7); 7.685(0.9); 7.681 (1.1); 7.664(0.5); 7.585(0.9); 7.567(2.1); 7.548(1.4); 7.453(1.3); 7.434(2.1); 7.415(1.0); 7.398(2.3); 7.379(1.9); 7.358(0.6); 7.351(3.0); 7.330(4.6); 7.310 (2.6); 7.303(0.6); 3.561(16.0); 3.382(0.9); 3.329(1.1); 3.215(0.5); 3.186(0.4); 2.675(0.4); 2.671(0.6); 2.666(0.5); 2.605(16.0); 2.506(62.3); 2.501(85.4); 2.497(67.1); 2.332(0.4); 2.328(0.6); 2.324(0.5); 0.000(7.4) |
| 28 | 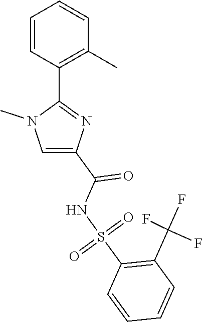 [SMILES: Cc1ccccc1-c1nc(C(=O)NS(=O)(=O)c2ccccc2C(F)(F)F)cn1C] | Example 28: ¹H-NMR(400.0 MHz, d₆-DMSO): d = 8.320(1.7); 8.300(1.8); 8.095(4.2); 7.938(0.3); 7.894(1.7); 7.875(2.2); 7.860(0.7); 7.842(1.7); 7.823 (1.2); 7.787(1.3); 7.768(1.5); 7.749(0.6); 7.727(0.8); 7.520(0.6); 7.501(1.7); 7.482(2.2); 7.460(2.1); 7.427(2.2); 7.408(1.4); 7.382(1.2); 7.363(1.8); 7.344 (0.7); 3.531(16.0); 3.391(233.0); 2.712(0.6); 2.676(1.8); 2.672(2.5); 2.667 (1.8); 2.542(124.8); 2.525(6.4); 2.507(272.5); 2.502(356.6); 2.498(265.4); 2.368(0.6); 2.334(1.7); 2.329(2.3); 2.325(1.8); 2.167(13.8); 1.259(0.4); 1.235 (0.9); 0.000(7.2) |
| 29 | 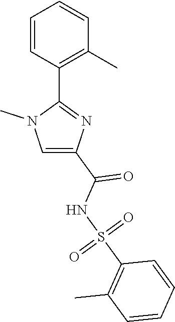 [SMILES: Cc1ccccc1-c1nc(C(=O)NS(=O)(=O)c2ccccc2C)cn1C] | Example 29: ¹H-NMR(400.0 MHz, d₆-DMSO): d = 8.058(5.7); 8.017(1.5); 8.014(1.7); 7.997(1.7); 7.995(1.7); 7.563(0.6); 7.560(0.7); 7.544(1.6); 7.541 (1.7); 7.525(1.1); 7.522(1.1); 7.462(0.5); 7.458(0.6); 7.439(1.7); 7.425(1.3); 7.421(1.7); 7.415(1.7); 7.391(3.3); 7.380(2.1); 7.372(3.3); 7.362(1.6); 7.339 (1.3); 7.320(1.4); 7.301(0.5); 3.839(0.6); 3.657(2.2); 3.483(16.0); 2.602(12.5); 2.542(61.7); 2.525(0.7); 2.520(0.9); 2.511(15.5); 2.507(32.3); 2.502(43.5); 2.498(32.7); 2.493(16.7); 2.329(0.3); 2.165(13.1); 0.000(1.0) |
| 30 | 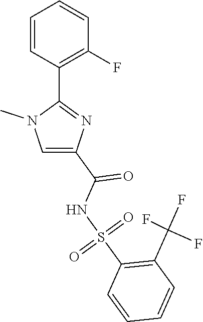 [SMILES: Cn1cc(C(=O)NS(=O)(=O)c2ccccc2C(F)(F)F)nc1-c1ccccc1F] | Example 30: ¹H-NMR(400.0 MHz, d₆-DMSO): d = 8.343(2.8); 8.323(3.0); 8.169(9.8); 7.979(2.3); 7.960(3.3); 7.939(1.1); 7.923(2.8); 7.904(2.1); 7.883 (2.2); 7.865(2.5); 7.846(0.8); 7.680(0.5); 7.676(0.8); 7.659(3.2); 7.643(4.9); 7.624(3.2); 7.464(1.8); 7.440(2.4); 7.417(3.6); 7.398(3.7); 7.379(1.7); 7.377 (1.6); 3.622(16.0); 3.620(15.9); 2.714(0.4); 2.561(0.3); 2.544(70.5); 2.509 (17.3); 2.505(22.8); 2.500(17.2); 2.370(0.4); 0.000(0.6) |
| 31 | 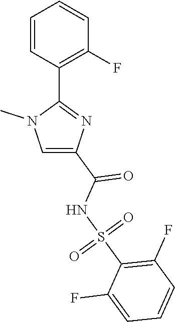 [SMILES: Cn1cc(C(=O)NS(=O)(=O)c2c(F)cccc2F)nc1-c1ccccc1F] | Example 31: ¹H-NMR(400.0 MHz, d₆-DMSO): d = 8.166(4.4); 7.693(1.1); 7.687(1.4); 7.682(1.7); 7.679(1.7); 7.675(2.6); 7.668(2.5); 7.663(3.0); 7.653 (1.5); 7.649(1.4); 7.645(1.6); 7.477(1.2); 7.453(1.7); 7.431(1.0); 7.425(1.6); 7.423(1.5); 7.404(2.4); 7.387(1.1); 7.385(1.1); 7.255(2.1); 7.233(3.4); 7.211 (1.8); 3.631(13.2); 3.547(16.0); 2.711(0.4); 2.676(0.5); 2.671(0.7); 2.667(0.5); 2.542(94.2); 2.525(1.7); 2.511(36.8); 2.507(74.0); 2.502(98.1); 2.498(73.5); 2.368(0.4); 2.333(0.5); 2.329(0.7); 2.325(0.5); 0.000(1.6) |
| 32 | 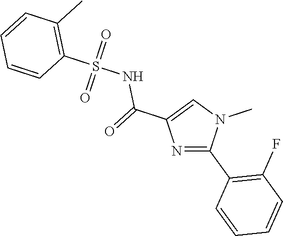 [SMILES: Cc1ccccc1S(=O)(=O)NC(=O)c1cn(C)c(-c2ccccc2F)n1] | Example 32: ¹H-NMR(400.0 MHz, d₆-DMSO): d = 8.116(7.6); 8.028(1.9); 8.025(2.2); 8.008(2.1); 8.005(2.3); 7.651(0.4); 7.647(0.6); 7.636(1.2); 7.632 (2.1); 7.614(3.8); 7.595(2.5); 7.586(0.9); 7.583(1.0); 7.567(2.1); 7.564(2.2); 7.549(1.4); 7.545(1.4); 7.452(1.3); 7.439(1.6); 7.433(2.4); 7.416(2.5); 7.414 (2.4); 7.397(4.3); 7.395(4.1); 7.379(4.5); 7.360(1.2); 7.358(1.2); 3.818(0.3); 3.776(0.4); 3.759(0.5); 3.736(0.5); 3.587(11.5); 3.584(12.0); 3.523(0.6); 3.483(0.5); 3.408(0.5); 2.608(16.0); 2.542(67.4); 2.525(0.7); 2.520(0.8); 2.511(15.4); 2.507(32.7); 2.502(44.2); 2.498(33.4); 2.494(17.3); 2.368(0.4); 2.329(0.4); 0.000(1.1) |
| 33 | 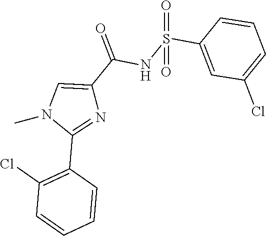 [SMILES: Cn1cc(C(=O)NS(=O)(=O)c2cccc(Cl)c2)nc1-c1ccccc1Cl] | Example 33: ¹H-NMR(400.0 MHz, d₆-DMSO): d = 8.122(5.8); 7.989(1.6); 7.985(3.0); 7.980(1.9); 7.940(1.5); 7.936(1.1); 7.923(1.3); 7.920(1.7); 7.917 (1.3); 7.783(1.1); 7.781(1.0); 7.778(1.0); 7.765(1.4); 7.763(1.5); 7.760(1.5); 7.758(1.3); 7.681(1.2); 7.678(1.4); 7.673(2.2); 7.660(2.7); 7.658(2.7); 7.653 (3.4); 7.633(2.3); 7.628(1.4); 7.615(1.4); 7.610(1.8); 7.596(1.4); 7.591(1.8); 7.577(2.6); 7.573(1.6); 7.538(1.6); 7.535(1.6); 7.519(1.7); 7.501(0.7); 7.498 (0.7); 3.513(16.0); 2.671(0.4); 2.525(1.0); 2.511(18.5); 2.507(37.4); 2.502 (50.2); 2.498(37.9); 2.494(19.3); 1.509(0.3); 0.008(1.4); 0.000(40.7); −0.008 (1.6) |
| 34 | 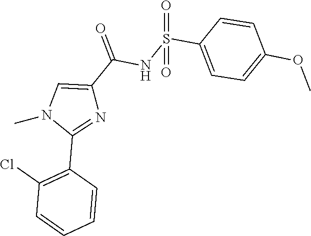 [SMILES: COc1ccc(S(=O)(=O)NC(=O)c2cn(C)c(-c3ccccc3Cl)n2)cc1] | Example 34: ¹H-NMR(400.0 MHz, d₆-DMSO): d = 8.077(5.2); 7.948(0.4); 7.940(4.1); 7.935(1.4); 7.929(0.5); 7.923(1.3); 7.918(4.5); 7.910(0.5); 7.662 (1.1); 7.660(1.2); 7.642(2.2); 7.640(2.2); 7.611(0.9); 7.606(1.1); 7.594(1.2); 7.589(1.5); 7.574(0.7); 7.569(1.0); 7.561(0.8); 7.556(1.0); 7.542(2.3); 7.537 (1.7); 7.521(1.5); 7.517(1.7); 7.500(1.4); 7.484(0.5); 7.481(0.5); 7.152(0.4); 7.144(4.3); 7.139(1.4); 7.127(1.2); 7.122(4.2); 7.114(0.5); 3.847(16.0); 3.823 (0.4); 3.493(13.4); 3.482(1.1); 2.525(0.5); 2.511(11.2); 2.507(23.1); 2.502 (31.3); 2.498(23.7); 2.493(12.1); 1.509(1.8); 0.008(0.9); 0.000(27.6); −0.009 (1.1) |
| 36 | 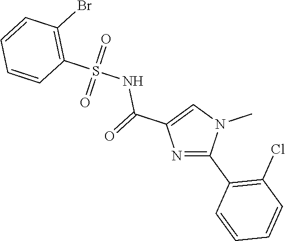 [SMILES: Cn1cc(C(=O)NS(=O)(=O)c2ccccc2Br)nc1-c1ccccc1Cl] | Example 36: ¹H-NMR(400.0 MHz, d₆-DMSO): d = 8.181(6.0); 8.161(1.9); 8.157(2.0); 7.832(1.9); 7.815(2.0); 7.813(2.2); 7.676(1.3); 7.656(3.2); 7.636 (1.9); 7.634(2.1); 7.628(1.3); 7.623(1.4); 7.617(1.4); 7.614(1.5); 7.610(1.6); 7.605(2.1); 7.600(1.8); 7.596(1.5); 7.590(1.8); 7.586(2.9); 7.581(3.1); 7.571 (1.8); 7.566(1.7); 7.552(0.7); 7.548(0.7); 7.534(1.5); 7.531(1.7); 7.514(2.0); 7.497(0.7); 7.494(0.8); 3.525(16.0); 3.499(0.3); 3.482(0.6); 2.671(0.4); 2.524 (0.8); 2.506(39.3); 2.502(53.7); 2.498(42.4); 2.329(0.4); 1.508(1.5); 0.008 (1.3); 0.000(40.0); −0.008(2.0) |
| 37 | 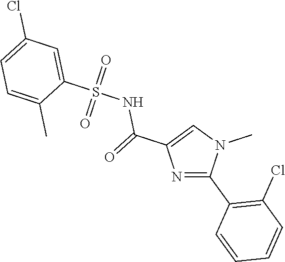 [SMILES: Cc1ccc(Cl)cc1S(=O)(=O)NC(=O)c1cn(C)c(-c2ccccc2Cl)n1] | Example 37: ¹H-NMR(400.0 MHz, d₆-DMSO): d = 8.128(5.7); 7.970(2.9); 7.964(3.0); 7.682(1.2); 7.662(2.9); 7.644(1.5); 7.638(1.5); 7.634(1.3); 7.629 (1.5); 7.623(1.9); 7.617(2.6); 7.609(2.7); 7.589(3.0); 7.538(1.5); 7.535(1.5); 7.519(1.9); 7.501(0.7); 7.498(0.7); 7.429(2.5); 7.408(2.1); 3.518(16.0); 2.578(13.7); 2.507(33.3); 2.502(46.0); 2.498(36.4); 2.415(0.5); 2.407(0.7); 2.329(0.5); 2.325(0.4); 2.075(14.9); 0.000(4.8) |
| 38 | 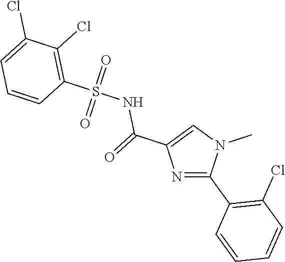 [SMILES: Cn1cc(C(=O)NS(=O)(=O)c2cccc(Cl)c2Cl)nc1-c1ccccc1Cl] | Example 38: ¹H-NMR(400.0 MHz, d₆-DMSO): d = 8.173(4.3); 8.123(1.8); 8.119(2.1); 8.103(2.1); 8.099(2.2); 8.034(0.5); 7.929(0.7); 7.925(1.6); 7.922 (1.7); 7.905(1.8); 7.901(1.9); 7.700(1.2); 7.698(1.3); 7.687(0.5); 7.680(2.5); 7.676(2.5); 7.655(1.1); 7.651(1.7); 7.647(1.7); 7.642(1.6); 7.638(1.4); 7.634 (2.7); 7.628(2.7); 7.624(2.0); 7.616(1.4); 7.612(0.6); 7.604(2.0); 7.584(3.6); 7.564(1.8); 7.554(1.5); 7.551(1.6); 7.533(2.3); 7.517(1.1); 7.514(1.1); 4.055 (0.4); 4.038(0.8); 4.020(0.8); 4.002(0.4); 3.545(16.0); 3.499(1.5); 3.482(1.8); 2.676(0.4); 2.671(0.6); 2.667(0.5); 2.525(1.5); 2.520(2.2); 2.511(31.4); 2.507 (65.1); 2.502(88.5); 2.498(67.4); 2.493(34.4); 2.333(0.4); 2.329(0.6); 2.324 (0.4); 2.075(1.1); 1.989(2.5); 1.509(4.7); 1.253(0.7); 1.237(0.7); 1.193(0.7); 1.175(1.3); 1.157(0.7); 0.905(0.7); 0.008(2.3); 0.000(71.9); −0.009(2.8) |
| 39 | 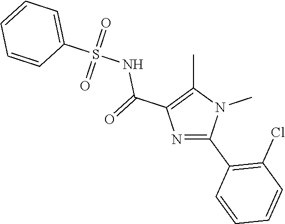 [SMILES: Cc1c(C(=O)NS(=O)(=O)c2ccccc2)nc(-c2ccccc2Cl)n1C] | Example 39: ¹H-NMR(400.0 MHz, d₆-DMSO): d = 8.013(2.9); 7.994(3.2); 7.708(0.5); 7.690(1.3); 7.671(2.4); 7.649(2.7); 7.634(2.2); 7.614(4.0); 7.602 (1.8); 7.597(2.7); 7.582(0.8); 7.577(1.0); 7.563(0.7); 7.558(1.0); 7.545(2.6); 7.540(2.0); 7.532(1.9); 7.529(2.0); 7.512(1.6); 7.496(0.5); 7.493(0.5); 3.753 (0.3); 3.739(0.4); 3.731(0.4); 3.719(0.4); 3.630(0.5); 3.517(0.8); 3.472(0.7); 3.406(0.6); 3.391(0.6); 3.343(15.2); 3.231(0.3); 2.996(0.8); 2.671(0.4); 2.542 (39.4); 2.507(41.0); 2.502(53.7); 2.498(41.5); 2.455(16.0); 2.329(0.4); 2.325 (0.3); 0.000(5.6) |
| 40 | 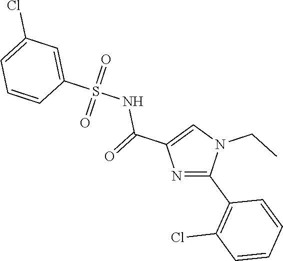 [SMILES: CCn1cc(C(=O)NS(=O)(=O)c2cccc(Cl)c2)nc1-c1ccccc1Cl] | Example 40: ¹H-NMR(400.0 MHz, d₆-DMSO): d = 8.198(5.7); 7.988(3.1); 7.983(5.4); 7.979(3.6); 7.934(2.9); 7.915(3.2); 7.773(2.0); 7.770(1.9); 7.752 (2.7); 7.750(2.7); 7.683(2.4); 7.681(2.4); 7.664(7.2); 7.645(5.0); 7.636(2.2); 7.631(2.5); 7.625(2.4); 7.618(2.8); 7.613(3.3); 7.597(3.2); 7.593(4.0); 7.578 (4.5); 7.574(3.2); 7.539(3.0); 7.536(2.8); 7.520(3.3); 7.502(1.2); 7.499(1.2); 4.126(0.3); 4.121(0.3); 4.098(0.4); 4.062(0.4); 4.035(0.5); 3.820(3.5); 3.802 (7.9); 3.784(8.2); 3.766(4.2); 3.738(2.6); 3.650(3.2); 3.313(0.5); 3.294(0.4); 2.996(1.0); 2.712(0.4); 2.675(0.6); 2.672(0.8); 2.542(66.6); 2.507(87.1); 2.503(110.0); 2.498(81.6); 2.368(0.3); 2.334(0.6); 2.329(0.7); 2.325(0.5); 1.233(7.6); 1.215(16.0); 1.196(7.3); 0.000(11.1) |
| 41 | 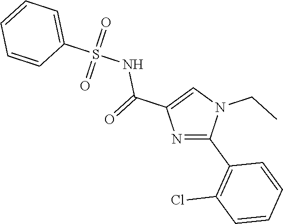 [SMILES: CCn1cc(C(=O)NS(=O)(=O)c2ccccc2)nc1-c1ccccc1Cl] | Example 41: ¹H-NMR(400.0 MHz, d₆-DMSO): d = 8.174(7.7); 8.004(6.2); 7.986(7.0); 7.982(5.4); 7.714(1.0); 7.695(3.3); 7.677(2.8); 7.667(2.7); 7.647 (5.1); 7.638(5.3); 7.618(8.6); 7.600(5.0); 7.597(4.7); 7.581(1.4); 7.576(1.8); 7.565(1.8); 7.560(2.2); 7.546(4.9); 7.541(3.7); 7.525(3.2); 7.522(3.3); 7.505 (3.2); 7.488(1.1); 7.485(1.1); 3.961(0.4); 3.943(0.4); 3.847(0.6); 3.803(2.7); 3.785(7.1); 3.767(7.3); 3.749(3.0); 3.460(9.5); 3.027(0.5); 2.996(1.3); 2.712 (0.4); 2.672(0.8); 2.668(0.7); 2.542(70.7); 2.525(2.4); 2.507(89.5); 2.503 (116.0); 2.499(88.8); 2.368(0.5); 2.334(0.7); 2.330(0.9); 2.325(0.7); 1.512 (0.7); 1.221(7.5); 1.203(16.0); 1.184(7.4); 0.000(2.3) |
| 42 | 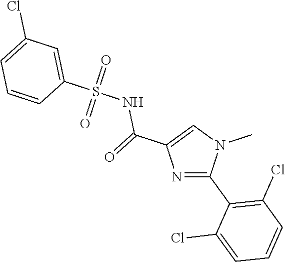 [SMILES: Cn1cc(C(=O)NS(=O)(=O)c2cccc(Cl)c2)nc1-c1c(Cl)cccc1Cl] | Example 42: ¹H-NMR(400.0 MHz, d₆-DMSO): d = 8.179(5.0); 7.997(3.3); 7.993(2.3); 7.962(1.8); 7.942(2.0); 7.810(1.3); 7.789(1.8); 7.706(2.0); 7.699 (3.5); 7.683(8.2); 7.677(3.9); 7.659(4.0); 7.644(1.8); 7.635(1.2); 7.620(0.8); 4.026(0.3); 4.015(0.4); 3.972(0.4); 3.854(0.5); 3.818(0.5); 3.799(0.5); 3.756 (0.5); 3.719(0.5); 3.706(0.5); 3.697(0.5); 3.649(0.5); 3.588(0.4); 3.535(0.3); 3.525(0.3); 3.508(0.4); 3.502(0.4); 3.474(16.0); 2.999(0.4); 2.544(32.5); 2.508(19.5); 2.504(24.4); 0.000(1.6) |
| 43 | 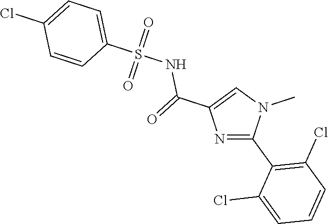 [SMILES: Cn1cc(C(=O)NS(=O)(=O)c2ccc(Cl)cc2)nc1-c1c(Cl)cccc1Cl] | Example 43: ¹H-NMR(400.0 MHz, d₆-DMSO): d = 8.163(5.4); 8.018(0.6); 8.012(4.8); 8.007(1.8); 7.995(1.8); 7.990(5.7); 7.984(0.8); 7.736(0.8); 7.729 (5.5); 7.725(2.0); 7.712(1.7); 7.708(4.9); 7.702(2.7); 7.695(3.0); 7.679(7.2); 7.678(7.4); 7.654(3.6); 7.639(2.0); 7.631(1.2); 7.615(0.9); 3.831(0.5); 3.748 (0.6); 3.746(0.6); 3.657(0.7); 3.642(0.7); 3.584(0.6); 3.519(0.5); 3.510(0.5); 3.467(16.0); 2.999(0.6); 2.545(41.5); 2.528(0.5); 2.510(14.8); 2.505(19.2); 2.501(14.4); 0.000(0.9) |
| 44 | 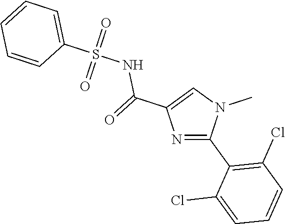 [SMILES: Cn1cc(C(=O)NS(=O)(=O)c2ccccc2)nc1-c1c(Cl)cccc1Cl] | Example 44: ¹H-NMR(400.0 MHz, d₆-DMSO): d = 8.149(5.1); 8.012(3.6); 7.993(4.1); 7.725(0.6); 7.707(2.1); 7.697(2.2); 7.691(3.7); 7.674(7.8); 7.649 (5.9); 7.632(4.0); 7.629(4.5); 7.610(2.2); 3.508(0.5); 3.458(16.0); 3.382(0.6); 3.338(0.6); 3.280(0.5); 2.997(0.6); 2.542(40.3); 2.506(25.1); 2.503(31.5); 2.499(24.5); −0.001(2.6) |
| 45 | 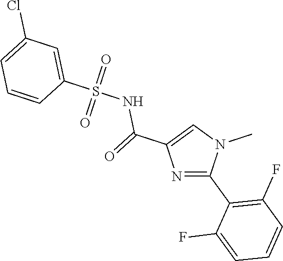 [SMILES: Cn1cc(C(=O)NS(=O)(=O)c2cccc(Cl)c2)nc1-c1c(F)cccc1F] | Example 45: ¹H-NMR(400.0 MHz, d₆-DMSO): d = 8.185(6.2); 7.997(2.1); 7.992(3.8); 7.988(2.6); 7.955(2.0); 7.935(2.2); 7.809(1.4); 7.806(1.4); 7.789 (1.9); 7.786(1.9); 7.752(0.4); 7.735(0.9); 7.730(0.9); 7.713(1.8); 7.691(3.2); 7.671(3.7); 7.651(1.5); 7.363(3.0); 7.342(4.6); 7.322(2.5); 3.567(16.0); 2.998 (0.5); 2.645(0.4); 2.543(38.9); 2.526(0.6); 2.508(24.4); 2.504(32.0); 2.499 (24.4); 0.000(3.9) |
| 46 | 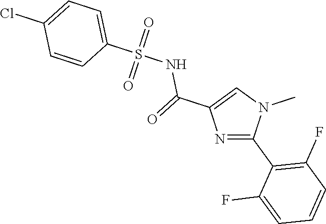 [SMILES: Cn1cc(C(=O)NS(=O)(=O)c2ccc(Cl)cc2)nc1-c1c(F)cccc1F] | Example 46: ¹H-NMR(400.0 MHz, d₆-DMSO): d = 8.167(6.0); 8.001(5.4); 7.979(6.4); 7.746(0.4); 7.729(1.8); 7.722(6.6); 7.705(2.8); 7.700(5.7); 7.693 (1.4); 7.687(1.2); 7.671(0.5); 7.358(3.0); 7.338(4.6); 7.317(2.5); 3.561(16.0); 2.998(0.6); 2.543(42.7); 2.526(0.7); 2.508(25.5); 2.504(32.9); 2.499(24.9); 0.000(4.2) |
| 47 | 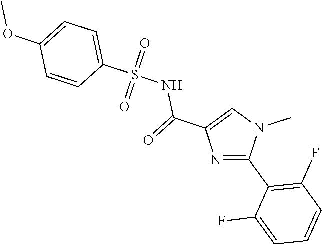 [SMILES: COc1ccc(S(=O)(=O)NC(=O)c2cn(C)c(-c3c(F)cccc3F)n2)cc1] | Example 47: ¹H-NMR(400.0 MHz, d₆-DMSO): d = 8.140(3.8); 7.935(4.0); 7.913(4.3); 7.722(0.7); 7.718(0.7); 7.701(1.3); 7.684(0.7); 7.680(0.8); 7.663 (0.3); 7.352(2.2); 7.331(3.5); 7.311(1.9); 7.143(4.1); 7.120(3.9); 3.845(16.0); 3.551(12.0); 3.507(0.4); 3.343(3.8); 2.997(0.5); 2.672(0.3); 2.542(37.9); 2.507(39.6); 2.503(50.5); 2.498(38.1); 2.330(0.3); 1.510(1.3); 1.255(0.4); 1.239(0.4); 0.903(0.3); 0.000(5.0) |
| 48 | 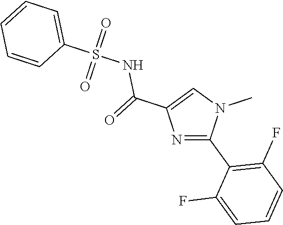 [SMILES: Cn1cc(C(=O)NS(=O)(=O)c2ccccc2)nc1-c1c(F)cccc1F] | Example 48: ¹H-NMR(400.0 MHz, d₆-DMSO): d = 8.159(6.3); 8.004(4.0); 7.986(4.8); 7.982(3.6); 7.742(0.4); 7.725(1.4); 7.722(1.5); 7.704(4.0); 7.685 (2.7); 7.666(0.5); 7.643(3.3); 7.623(4.7); 7.605(1.9); 7.362(0.6); 7.355(3.0); 7.335(4.5); 7.314(2.5); 7.307(0.6); 3.555(16.0); 3.507(0.4); 3.485(0.4); 3.375(0.6); 2.542(5.9); 2.525(0.5); 2.507(26.3); 2.503(34.6); 2.498(26.1); 1.510(0.5); 0.000(4.1) |
| 49 | 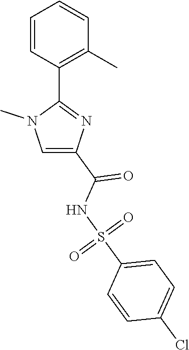 [SMILES: Cc1ccccc1-c1nc(C(=O)NS(=O)(=O)c2ccc(Cl)cc2)cn1C] | Example 49: ¹H-NMR(400.0 MHz, d₆-DMSO): d = 8.053(5.6); 7.974(0.7); 7.968(4.8); 7.963(1.9); 7.951(1.9); 7.946(5.6); 7.668(5.3); 7.664(2.1); 7.647 (4.6); 7.482(0.6); 7.479(0.7); 7.460(1.7); 7.445(1.3); 7.442(1.5); 7.401(3.5); 7.384(3.6); 7.381(3.5); 7.351(1.5); 7.333(1.7); 7.315(0.6); 4.240(0.4); 4.163 (0.6); 4.131(0.6); 4.020(0.8); 4.005(0.8); 3.976(0.8); 3.966(0.8); 3.957(0.8); 3.949(0.8); 3.936(0.8); 3.891(0.8); 3.488(16.0); 2.542(8.0); 2.507(21.8); 2.503(28.4); 2.499(21.6); 2.158(14.1); 0.000(2.2) |
| 50 | 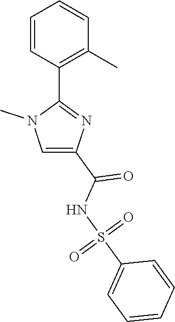 [SMILES: Cc1ccccc1-c1nc(C(=O)NS(=O)(=O)c2ccccc2)cn1C] | Example 50: ¹H-NMR(400.0 MHz, d₆-DMSO): d = 8.032(1.6); 7.988(1.4); 7.970(1.6); 7.967(1.3); 7.672(0.7); 7.653(0.6); 7.618(1.2); 7.599(1.6); 7.581 (0.6); 7.438(0.6); 7.423(0.5); 7.420(0.6); 7.388(1.0); 7.368(0.9); 7.352(1.0); 7.335(0.7); 7.316(0.7); 3.471(16.0); 3.454(13.7); 3.292(0.4); 2.542(2.8); 2.507(30.4); 2.503(40.0); 2.498(30.7); 2.157(6.1) |
| 51 | 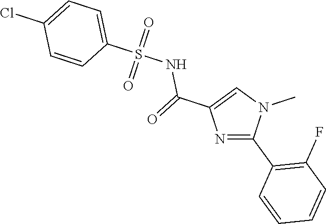 [SMILES: Cn1cc(C(=O)NS(=O)(=O)c2ccc(Cl)cc2)nc1-c1ccccc1F] | Example 51: ¹H-NMR(400.0 MHz, d₆-DMSO): d = 8.098(5.3); 7.998(0.6); 7.992(5.0); 7.988(1.9); 7.975(1.8); 7.970(6.0); 7.964(0.9); 7.716(0.8); 7.710 (5.6); 7.705(2.1); 7.693(1.7); 7.688(4.9); 7.658(0.4); 7.653(0.5); 7.644(0.5); 7.639(1.1); 7.634(2.3); 7.616(3.0); 7.599(1.4); 7.444(1.1); 7.424(1.2); 7.418 (1.5); 7.402(1.8); 7.399(2.1); 7.383(2.4); 7.364(1.1); 7.362(1.0); 3.587(15.7); 3.584(16.0); 2.997(0.4); 2.673(0.3); 2.543(16.6); 2.526(1.1); 2.512(18.7); 2.508(37.2); 2.504(48.8); 2.499(36.7); 2.330(0.3); 0.000(4.0) |
| 52 | 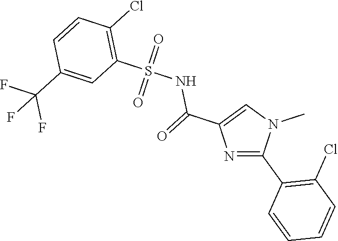 [SMILES: Cn1cc(C(=O)NS(=O)(=O)c2cc(C(F)(F)F)ccc2Cl)nc1-c1ccccc1Cl] | Example 52: ¹H-NMR(400.0 MHz, d₆-DMSO): d = 8.331(2.4); 8.326(2.5); 8.317(0.4); 8.179(3.8); 7.968(1.1); 7.963(1.1); 7.947(1.4); 7.942(1.4); 7.930 (0.7); 7.822(2.3); 7.802(1.8); 7.721(1.1); 7.701(3.7); 7.687(1.7); 7.682(2.6); 7.679(2.4); 7.668(2.2); 7.664(1.3); 7.648(0.9); 7.644(0.7); 7.637(0.4); 7.576 (1.4); 7.573(1.4); 7.558(2.1); 7.554(2.0); 7.539(0.8); 7.536(0.9); 7.518(0.4); 7.512(0.5); 3.569(16.0); 3.499(0.9); 3.483(1.9); 2.676(0.4); 2.671(0.6); 2.667 (0.5); 2.524(1.3); 2.511(37.3); 2.507(75.9); 2.502(101.2); 2.498(76.0); 2.493 (38.8); 2.333(0.5); 2.329(0.7); 2.324(0.5); 1.509(5.1); 1.253(0.4); 1.237(0.4); 0.905(0.4); 0.146(0.5); 0.008(3.5); 0.000(108.9); −0.009(4.9); −0.150(0.5) |
| 53 | 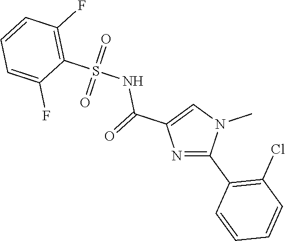 [SMILES: Cn1cc(C(=O)NS(=O)(=O)c2c(F)cccc2F)nc1-c1ccccc1Cl] | Example 53: ¹H-NMR(400.0 MHz, d₆-DMSO): d = 7.661(0.4); 7.631(2.2); 7.612(3.3); 7.568(1.0); 7.563(1.0); 7.551(1.5); 7.546(1.8); 7.532(1.0); 7.526 (1.3); 7.511(0.9); 7.491(3.8); 7.472(2.3); 7.455(1.2); 7.441(0.8); 7.213(0.3); 7.104(0.7); 7.087(0.7); 7.080(0.7); 7.043(1.6); 7.024(2.1); 3.671(0.4); 3.633 (0.6); 3.576(0.8); 3.506(1.1); 3.460(16.0); 2.731(0.4); 2.676(0.6); 2.671(0.8); 2.666(0.6); 2.524(1.8); 2.519(2.9); 2.511(40.8); 2.506(83.5); 2.502(111.9); 2.497(82.7); 2.493(41.3); 2.333(0.6); 2.329(0.8); 2.324(0.6); 0.859(0.7); 0.841(1.3); 0.823(0.6); 0.146(0.5); 0.008(3.9); 0.000(114.9); −0.009(4.5); −0.150(0.5) |
| 54 | 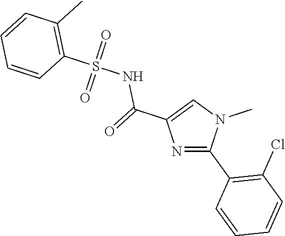 [SMILES: Cc1ccccc1S(=O)(=O)NC(=O)c1cn(C)c(-c2ccccc2Cl)n1] | Example 54: ¹H-NMR(400.0 MHz, d₆-DMSO): d = 8.089(2.8); 8.020(1.6); 8.003(1.6); 8.000(1.7); 7.929(0.4); 7.666(1.2); 7.663(1.2); 7.646(2.4); 7.644 (2.3); 7.613(1.0); 7.608(1.2); 7.595(1.4); 7.591(1.7); 7.575(2.1); 7.570(2.9); 7.555(3.8); 7.551(3.0); 7.536(1.0); 7.533(1.0); 7.523(1.7); 7.520(1.8); 7.511 (0.5); 7.502(1.6); 7.486(0.6); 7.483(0.7); 7.442(0.9); 7.423(1.5); 7.404(0.7); 7.388(1.7); 7.370(1.4); 3.500(16.0); 3.482(1.6); 2.750(0.8); 2.671(0.4); 2.606 (12.4); 2.524(0.8); 2.520(1.3); 2.511(19.4); 2.507(40.0); 2.502(53.6); 2.498 (40.2); 2.493(20.4); 2.329(0.4); 2.075(1.2); 1.508(3.5); 0.008(1.9); 0.000 (59.4); −0.009(2.4) |
| 55 | 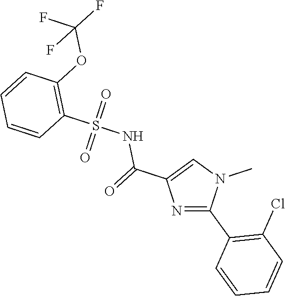 [SMILES: Cn1cc(C(=O)NS(=O)(=O)c2ccccc2OC(F)(F)F)nc1-c1ccccc1Cl] | Example 55: ¹H-NMR(400.0 MHz, d₆-DMSO): d = 8.140(3.4); 8.112(1.7); 8.108(1.8); 8.092(1.8); 8.088(1.9); 7.809(0.7); 7.806(0.7); 7.787(1.5); 7.770 (0.9); 7.766(1.0); 7.684(1.3); 7.666(2.8); 7.637(1.2); 7.633(1.3); 7.619(1.5); 7.615(2.7); 7.604(1.5); 7.600(2.1); 7.595(3.1); 7.585(2.8); 7.581(1.9); 7.575 (1.2); 7.555(1.4); 7.542(2.1); 7.539(2.5); 7.523(2.0); 7.505(0.7); 7.502(0.7); 3.527(16.0); 3.499(0.4); 3.482(0.6); 2.671(0.5); 2.524(1.0); 2.507(56.2); 2.502(74.9); 2.498(57.4); 2.333(0.4); 2.329(0.5); 2.325(0.4); 1.509(1.6); 0.008(2.2); 0.000(73.2); −0.008(3.6); −0.150(0.4) |
| 56 | 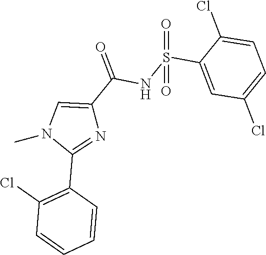 [SMILES: Cn1cc(C(=O)NS(=O)(=O)c2cc(Cl)ccc2Cl)nc1-c1ccccc1Cl] | Example 56: ¹H-NMR(400.0 MHz, d₆-DMSO): d = 8.175(5.6); 8.053(3.3); 8.047(3.4); 7.710(2.3); 7.704(1.4); 7.692(3.3); 7.689(4.2); 7.682(2.5); 7.670 (3.7); 7.651(4.9); 7.632(5.3); 7.610(2.5); 7.565(1.4); 7.562(1.4); 7.544(2.0); 7.528(0.8); 7.525(0.8); 3.556(16.0); 3.483(0.5); 2.671(0.4); 2.524(0.8); 2.511 (26.2); 2.507(52.5); 2.502(69.6); 2.498(52.3); 2.493(27.0); 2.333(0.4); 2.329 (0.5); 2.324(0.4); 2.075(0.4); 1.509(1.4); 0.008(2.4); 0.000(73.9); −0.009(3.6); −0.150(0.4) |
| 57 | 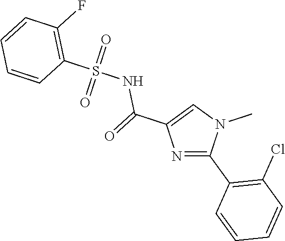 [SMILES: Cn1cc(C(=O)NS(=O)(=O)c2ccccc2F)nc1-c1ccccc1Cl] | Example 57: ¹H-NMR(400.0 MHz, d₆-DMSO): d = 8.151(4.2); 7.985(0.6); 7.981(0.8); 7.965(1.3); 7.962(1.5); 7.947(0.8); 7.942(0.8); 7.755(0.4); 7.746 (0.4); 7.739(0.8); 7.721(0.8); 7.715(0.5); 7.707(0.5); 7.703(0.4); 7.680(1.2); 7.677(1.2); 7.660(2.5); 7.630(1.0); 7.626(1.3); 7.612(1.3); 7.608(1.8); 7.599 (1.3); 7.595(1.4); 7.588(1.4); 7.580(2.6); 7.576(1.6); 7.536(1.5); 7.532(1.6); 7.516(1.7); 7.499(0.7); 7.495(0.7); 7.441(1.9); 7.422(2.4); 7.413(1.4); 7.405 (1.1); 7.403(1.1); 7.392(0.9); 3.518(16.0); 2.511(19.8); 2.507(44.1); 2.502 (61.7); 2.497(48.5); 2.493(26.5); 2.333(0.4); 2.329(0.6); 2.324(0.5); 0.008 (1.0); 0.000(65.1); −0.008(4.1); −0.150(0.4) |
| 58 | 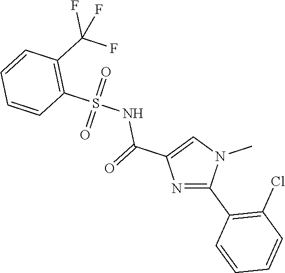 [SMILES: Cn1cc(C(=O)NS(=O)(=O)c2ccccc2C(F)(F)F)nc1-c1ccccc1Cl] | Example 58: ¹H-NMR(400.0 MHz, d₆-DMSO): d = 8.337(1.7); 8.317(2.0); 8.153(3.4); 7.968(1.3); 7.949(1.9); 7.930(0.7); 7.915(1.5); 7.895(1.1); 7.872 (1.2); 7.854(1.3); 7.836(0.5); 7.690(1.1); 7.688(1.2); 7.670(2.7); 7.643(1.0); 7.638(1.4); 7.625(1.3); 7.620(2.2); 7.618(2.4); 7.614(1.4); 7.597(2.7); 7.546 (1.5); 7.543(1.6); 7.526(1.9); 7.509(0.8); 7.506(0.8); 3.533(16.0); 3.499(0.4); 3.482(0.6); 2.676(0.4); 2.671(0.6); 2.667(0.5); 2.525(1.5); 2.511(34.4); 2.507 (70.2); 2.502(94.0); 2.498(71.7); 2.333(0.5); 2.329(0.6); 2.325(0.5); 2.075 (1.3); 1.508(1.5); 0.146(0.4); 0.008(3.1); 0.000(94.0); −0.008(4.2); −0.150 (0.4) |
| 59 | 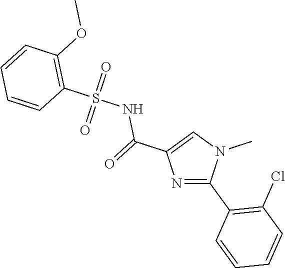 [SMILES: COc1ccccc1S(=O)(=O)NC(=O)c1cn(C)c(-c2ccccc2Cl)n1] | Example 59: ¹H-NMR(400.0 MHz, d₆-DMSO): d = 8.151(4.9); 7.928(0.4); 7.904(1.5); 7.900(1.7); 7.884(1.7); 7.880(1.8); 7.687(0.7); 7.683(0.8); 7.669 (1.9); 7.666(1.6); 7.653(2.5); 7.651(2.6); 7.644(1.2); 7.618(0.9); 7.613(1.1); 7.600(1.3); 7.595(1.7); 7.580(0.8); 7.575(2.0); 7.571(1.3); 7.556(2.5); 7.552 (1.8); 7.529(1.6); 7.526(1.7); 7.517(0.4); 7.510(1.8); 7.493(0.7); 7.490(0.7); 7.240(2.1); 7.219(1.9); 7.163(1.2); 7.145(2.0); 7.127(1.0); 7.125(1.0); 3.849 (16.0); 3.509(14.5); 3.482(1.3); 3.439(0.4); 3.414(0.4); 3.344(0.5); 3.329 (0.5); 2.524(0.7); 2.511(13.6); 2.507(27.8); 2.502(37.6); 2.498(29.5); 2.075 (0.4); 1.508(2.9); 0.008(1.4); 0.000(38.7); −0.008(2.4) |
| 60 | 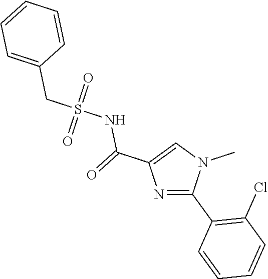 [SMILES: Cn1cc(C(=O)NS(=O)(=O)Cc2ccccc2)nc1-c1ccccc1Cl] | Example 60: ¹H-NMR(400.0 MHz, d₆-DMSO): d = 8.185(2.5); 7.670(1.3); 7.668(1.4); 7.650(2.6); 7.648(2.6); 7.615(1.0); 7.610(1.2); 7.597(1.4); 7.592 (1.8); 7.575(1.6); 7.572(2.2); 7.556(2.7); 7.552(1.9); 7.525(1.7); 7.522(1.8); 7.506(1.8); 7.488(0.7); 7.485(0.7); 7.385(1.8); 7.381(2.2); 7.368(6.6); 7.351 (4.1); 7.342(2.6); 7.332(1.7); 7.326(1.2); 4.801(5.9); 3.527(16.0); 3.498(0.7); 3.481(1.0); 3.348(1.3); 2.675(0.5); 2.671(0.6); 2.667(0.5); 2.524(1.6); 2.506 (68.9); 2.502(90.6); 2.497(68.5); 2.333(0.5); 2.329(0.6); 2.324(0.5); 1.508 (1.2); 0.146(0.4); 0.008(3.2); 0.000(81.3); −0.008(4.1); −0.150(0.4) |
| 61 | 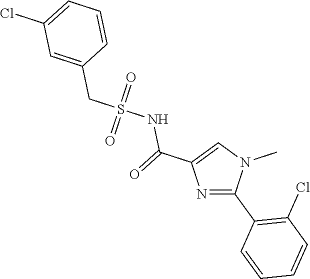 [SMILES: Cn1cc(C(=O)NS(=O)(=O)Cc2cccc(Cl)c2)nc1-c1ccccc1Cl] | Example 61: ¹H-NMR(400.0 MHz, d₆-DMSO): d = 8.188(3.5); 7.669(1.4); 7.650(2.7); 7.618(1.0); 7.613(1.2); 7.600(1.4); 7.595(1.8); 7.576(2.0); 7.558 (2.7); 7.554(1.9); 7.527(1.7); 7.525(1.7); 7.507(1.8); 7.490(0.6); 7.488(0.7); 7.457(0.8); 7.437(2.7); 7.433(2.1); 7.427(2.3); 7.409(5.0); 7.389(1.1); 7.302 (2.0); 7.284(1.6); 4.835(6.3); 3.530(16.0); 3.499(0.3); 2.675(0.4); 2.671(0.5); 2.506(58.6); 2.502(77.6); 2.498(59.3); 2.333(0.4); 2.329(0.5); 2.324(0.4); 0.008(2.4); 0.000(65.2); −0.008(3.4) |
| 62 | 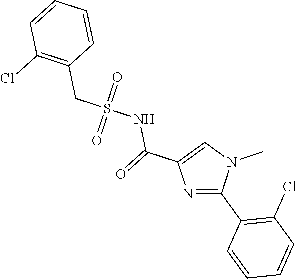 [SMILES: Cn1cc(C(=O)NS(=O)(=O)Cc2ccccc2Cl)nc1-c1ccccc1Cl] | Example 62: ¹H-NMR(400.0 MHz, d₆-DMSO): d = 8.197(3.2); 7.676(1.2); 7.674(1.3); 7.656(2.5); 7.654(2.5); 7.620(1.0); 7.615(1.2); 7.602(1.3); 7.598 (1.8); 7.587(1.3); 7.583(1.8); 7.578(1.5); 7.568(2.6); 7.564(1.7); 7.528(2.9); 7.523(1.8); 7.510(2.9); 7.505(2.3); 7.499(1.5); 7.494(1.8); 7.481(1.8); 7.475 (1.9); 7.430(0.5); 7.425(0.7); 7.412(1.7); 7.406(1.5); 7.394(2.8); 7.389(2.2); 7.376(1.6); 7.372(1.4); 7.358(0.5); 7.354(0.4); 4.967(6.6); 3.537(16.0); 3.498 (0.7); 3.481(0.8); 3.413(0.6); 3.402(0.6); 3.387(0.6); 3.358(0.6); 3.322(0.5); 3.313(0.5); 3.302(0.5); 3.244(0.4); 2.675(0.5); 2.671(0.6); 2.666(0.5); 2.524 (1.5); 2.519(2.4); 2.511(35.9); 2.506(73.5); 2.502(98.0); 2.497(73.2); 2.493 (36.9); 2.333(0.5); 2.329(0.7); 2.324(0.5); 1.508(1.1); 0.146(0.6); 0.008(4.5); 0.000(127.4); −0.009(5.2); −0.150(0.6) |
| 63 | 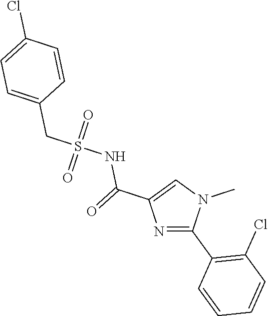 [SMILES: Cn1cc(C(=O)NS(=O)(=O)Cc2ccc(Cl)cc2)nc1-c1ccccc1Cl] | Example 63: ¹H-NMR(400.0 MHz, d₆-DMSO): d = 8.188(4.2); 7.673(1.2); 7.670(1.3); 7.653(2.5); 7.650(2.4); 7.617(1.0); 7.612(1.2); 7.599(1.4); 7.595 (1.8); 7.578(1.4); 7.574(2.0); 7.558(2.6); 7.554(1.7); 7.527(1.7); 7.523(1.7); 7.508(1.6); 7.490(0.7); 7.487(0.6); 7.473(0.4); 7.466(3.7); 7.462(1.4); 7.450 (1.6); 7.445(5.9); 7.439(0.9); 7.370(0.7); 7.364(5.3); 7.348(1.3); 7.343(3.6); 4.819(6.2); 3.529(16.0); 3.498(0.5); 3.482(0.4); 3.457(0.4); 3.438(0.4); 3.398 (0.4); 3.366(0.4); 3.350(0.4); 3.305(0.3); 2.676(0.4); 2.671(0.5); 2.666(0.4); 2.524(1.3); 2.520(1.9); 2.511(28.5); 2.507(59.1); 2.502(79.0); 2.497(58.2); 2.493(28.6); 2.333(0.4); 2.329(0.5); 2.324(0.4); 1.508(0.3); 1.202(0.8); 0.146(0.5); 0.008(3.6); 0.000(108.7); −0.009(4.1); −0.150(0.5) |
| 64 | 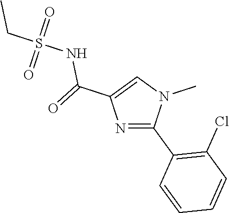 [SMILES: CCS(=O)(=O)NC(=O)c1cn(C)c(-c2ccccc2Cl)n1] | Example 64: ¹H-NMR(400.0 MHz, d₆-DMSO): d = 8.184(5.1); 7.674(1.1); 7.672(1.2); 7.654(2.4); 7.652(2.2); 7.619(1.0); 7.614(1.2); 7.601(1.3); 7.596 (1.7); 7.585(1.2); 7.581(1.6); 7.577(1.4); 7.565(2.5); 7.561(1.5); 7.531(1.6); 7.527(1.6); 7.511(1.6); 7.494(0.6); 7.491(0.6); 3.529(16.0); 3.480(1.2); 3.462 (3.6); 3.444(3.7); 3.425(1.3); 3.350(0.5); 3.288(0.4); 3.277(0.4); 3.268(0.3); 2.737(0.4); 2.671(0.3); 2.524(0.8); 2.520(1.3); 2.511(19.1); 2.507(39.0); 2.502(51.9); 2.497(38.0); 2.493(18.6); 2.329(0.4); 1.258(4.0); 1.239(8.9); 1.221(3.9); 0.008(2.4); 0.000(71.2); −0.009(2.6); −0.150(0.3) |
| 65 | 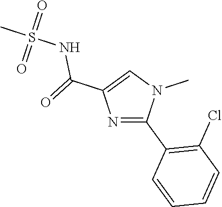 [SMILES: Cn1cc(C(=O)NS(C)(=O)=O)nc1-c1ccccc1Cl] | Example 65: ¹H-NMR(400.0 MHz, d₆-DMSO): d = 8.187(5.1); 7.676(1.3); 7.673(1.4); 7.665(0.6); 7.655(2.5); 7.653(2.3); 7.632(0.4); 7.621(1.2); 7.616 (1.4); 7.603(1.6); 7.598(1.8); 7.583(1.1); 7.578(2.2); 7.574(1.6); 7.560(2.6); 7.555(1.8); 7.532(1.9); 7.529(1.8); 7.514(1.6); 7.495(0.7); 7.492(0.7); 3.591 (0.5); 3.531(16.0); 3.509(1.1); 3.488(1.7); 3.351(3.3); 3.321(5.1); 3.310 (18.1); 3.175(0.7); 3.134(0.5); 2.891(0.4); 2.732(0.3); 2.676(0.4); 2.671(0.5); 2.667(0.4); 2.511(42.6); 2.507(63.3); 2.502(73.7); 2.498(51.9); 2.493(25.2); 2.334(0.5); 2.329(0.5); 2.325(0.4); 0.013(7.2); 0.011(7.2); 0.008(8.0); 0.000 (66.0); −0.009(2.7) |
| 66 | 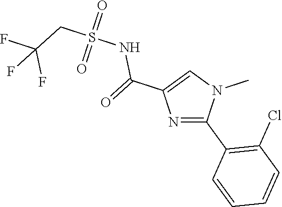 [SMILES: Cn1cc(C(=O)NS(=O)(=O)CC(F)(F)F)nc1-c1ccccc1Cl] | Example 66: ¹H-NMR(400.0 MHz, d₆-DMSO): d = 8.212(3.0); 7.722(1.2); 7.704(2.9); 7.689(1.9); 7.682(1.6); 7.677(1.7); 7.671(2.6); 7.663(2.4); 7.643 (1.0); 7.639(0.8); 7.578(1.5); 7.575(1.5); 7.559(2.0); 7.557(2.0); 7.541(0.8); 7.538(0.8); 4.591(0.8); 4.566(2.2); 4.541(2.3); 4.515(0.9); 4.065(0.7); 3.923 (1.0); 3.847(0.9); 3.751(0.6); 3.710(0.5); 3.575(16.0); 3.517(0.4); 3.488(0.9); 2.722(0.3); 2.676(0.4); 2.671(0.6); 2.667(0.5); 2.507(70.1); 2.502(90.4); 2.498(67.5); 2.333(0.5); 2.329(0.6); 2.325(0.5); 0.008(2.3); 0.000(54.5); −0.008(2.6) |
| 67 | 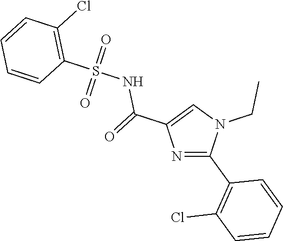 [SMILES: CCn1cc(C(=O)NS(=O)(=O)c2ccccc2Cl)nc1-c1ccccc1Cl] | Example 67: ¹H-NMR(400.0 MHz, d₆-DMSO): d = 8.267(7.7); 8.155(2.9); 8.151(3.2); 8.135(3.3); 8.132(3.4); 7.698(0.7); 7.695(0.8); 7.678(4.6); 7.658 (8.0); 7.653(4.9); 7.648(5.6); 7.632(3.3); 7.627(3.0); 7.609(5.4); 7.599(2.7); 7.589(5.4); 7.584(5.8); 7.573(1.7); 7.569(1.4); 7.534(2.7); 7.532(2.8); 7.515 (3.4); 7.498(1.2); 7.495(1.3); 5.757(12.2); 3.831(2.2); 3.813(6.5); 3.794(6.6); 3.776(2.3); 2.676(0.4); 2.671(0.5); 2.524(1.1); 2.506(63.5); 2.502(83.9); 2.498(64.4); 2.329(0.6); 2.325(0.5); 1.249(7.4); 1.231(16.0); 1.213(7.4); 0.146(0.6); 0.008(4.7); 0.000(127.1); −0.008(6.6); −0.150(0.7) |
| 68 | 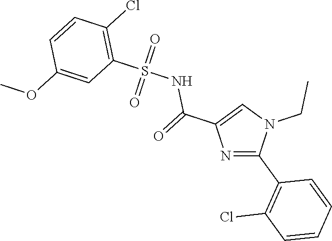 [SMILES: CCn1cc(C(=O)NS(=O)(=O)c2cc(OC)ccc2Cl)nc1-c1ccccc1Cl] | Example 68: ¹H-NMR(400.0 MHz, d₆-DMSO): d = 8.259(1.7); 7.683(1.0); 7.681(1.0); 7.663(2.4); 7.635(0.9); 7.630(1.1); 7.612(2.8); 7.606(3.9); 7.598 (3.5); 7.593(3.2); 7.548(1.9); 7.538(1.5); 7.535(1.5); 7.526(2.3); 7.519(2.0); 7.501(0.6); 7.498(0.6); 7.259(1.1); 7.251(1.1); 7.237(1.0); 7.229(1.0); 4.026 (0.4); 3.846(16.0); 3.836(1.6); 3.817(3.2); 3.799(3.2); 3.781(1.2); 2.671(0.3); 2.507(43.1); 2.502(56.2); 2.498(43.4); 2.334(0.3); 2.329(0.4); 2.325(0.3); 2.075(0.5); 1.251(3.5); 1.233(7.6); 1.215(3.5); 0.008(2.7); 0.000(66.0); −0.150(0.3) |
| 69 | 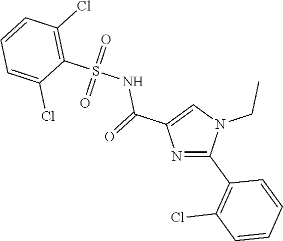 [SMILES: CCn1cc(C(=O)NS(=O)(=O)c2c(Cl)cccc2Cl)nc1-c1ccccc1Cl] | Example 69: ¹H-NMR(400.0 MHz, d₆-DMSO): d = 8.255(3.9); 7.701(2.3); 7.681(5.3); 7.657(6.1); 7.638(8.7); 7.619(2.0); 7.599(5.2); 7.581(10.7); 7.554 (2.9); 7.532(6.0); 7.514(3.9); 7.509(2.6); 7.491(1.5); 3.856(4.7); 3.838(8.9); 3.819(8.9); 3.802(4.5); 3.510(0.4); 3.467(0.3); 2.671(1.0); 2.502(162.2); 2.328(1.1); 1.263(7.6); 1.245(16.0); 1.227(7.5); 0.146(0.4); −0.001(70.1); −0.150(0.4) |
| 70 | 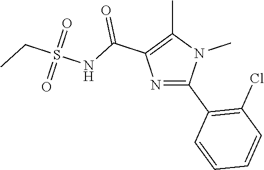 [SMILES: CCS(=O)(=O)NC(=O)c1nc(-c2ccccc2Cl)n(C)c1C] | Example 70: ¹H-NMR(400.0 MHz, d₆-DMSO): d = 7.675(2.2); 7.673(2.3); 7.655(4.2); 7.653(4.1); 7.618(1.7); 7.613(2.0); 7.601(2.3); 7.596(2.9); 7.581 (1.3); 7.576(2.2); 7.573(1.8); 7.568(1.9); 7.553(4.5); 7.549(3.1); 7.531(3.0); 7.528(3.1); 7.511(2.7); 7.495(0.9); 7.492(1.0); 3.804(0.4); 3.789(0.4); 3.742 (0.4); 3.683(0.6); 3.635(0.8); 3.559(1.6); 3.485(5.1); 3.467(10.0); 3.449 (12.1); 3.430(11.8); 3.385(75.3); 3.359(100.2); 3.059(0.6); 2.996(1.0); 2.681 (0.7); 2.676(1.4); 2.672(1.9); 2.667(1.4); 2.663(0.8); 2.557(31.9); 2.542 (39.3); 2.525(4.5); 2.520(7.1); 2.512(104.7); 2.507(216.2); 2.503(286.7); 2.498(211.4); 2.494(105.3); 2.334(1.4); 2.330(1.9); 2.325(1.4); 1.256(7.1); 1.238(16.0); 1.219(6.8); 0.008(0.8); 0.000(26.4); −0.009(1.0) |
| 71 | 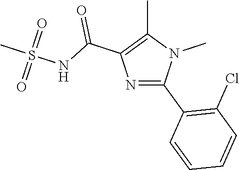 [SMILES: Cc1c(C(=O)NS(C)(=O)=O)nc(-c2ccccc2Cl)n1C] | Example 71: ¹H-NMR(400.0 MHz, d₆-DMSO): d = 7.674(1.2); 7.655(2.2); 7.653(2.2); 7.620(0.8); 7.614(1.0); 7.602(1.1); 7.597(1.5); 7.582(0.6); 7.577 (0.9); 7.563(0.6); 7.558(0.9); 7.544(2.5); 7.539(1.9); 7.531(1.8); 7.528(1.7); 7.512(1.4); 7.495(0.5); 7.492(0.4); 3.560(0.6); 3.385(18.6); 3.309(17.4); 3.207(0.8); 3.148(0.4); 3.134(0.4); 2.676(0.4); 2.671(0.5); 2.667(0.4); 2.568 (16.0); 2.542(5.7); 2.525(1.1); 2.511(27.3); 2.507(55.3); 2.502(72.8); 2.498 (54.4); 2.494(28.1); 2.334(0.4); 2.329(0.5); 2.324(0.4); 0.000(4.9) |
| 72 | 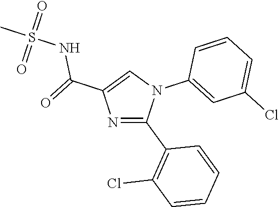 [SMILES: CS(=O)(=O)NC(=O)c1cn(-c2cccc(Cl)c2)c(-c2ccccc2Cl)n1] | Example 72: ¹H-NMR(400.0 MHz, d₆-DMSO): d = 8.548(16.0); 7.704(2.9); 7.698(3.5); 7.694(1.8); 7.683(3.8); 7.680(4.5); 7.546(0.9); 7.541(1.1); 7.527 (2.8); 7.521(2.1); 7.517(1.1); 7.508(4.5); 7.504(4.2); 7.492(5.5); 7.485(7.7); 7.482(8.8); 7.477(6.6); 7.466(5.8); 7.462(7.4); 7.458(6.1); 7.437(10.8); 7.432(4.4); 7.418(6.7); 7.398(2.8); 7.202(3.6); 7.200(3.4); 7.198(3.1); 7.186 (2.4); 7.181(3.2); 7.178(2.5); 3.817(0.4); 3.533(0.5); 3.361(41.0); 3.185(0.6); 2.998(1.0); 2.712(0.4); 2.543(87.7); 2.526(0.9); 2.508(31.0); 2.503(40.8); 2.499(30.7); 2.369(0.4); 0.000(1.8) |
| 73 | 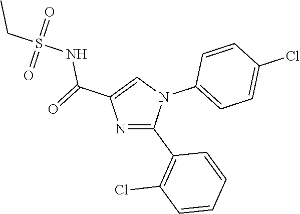 [SMILES: CCS(=O)(=O)NC(=O)c1cn(-c2ccc(Cl)cc2)c(-c2ccccc2Cl)n1] | Example 73: ¹H-NMR(400.0 MHz, d₆-DMSO): d = 8.501(11.3); 7.682(3.5); 7.663(3.6); 7.535(0.8); 7.531(0.9); 7.521(1.1); 7.512(4.1); 7.503(8.8); 7.498 (6.6); 7.493(4.4); 7.481(13.8); 7.479(12.6); 7.461(4.9); 7.443(1.3); 7.439 (1.2); 7.302(10.2); 7.280(8.2); 3.527(2.4); 3.509(7.0); 3.490(7.2); 3.472(2.9); 3.343(49.1); 2.996(1.0); 2.672(0.8); 2.542(48.1); 2.507(100.6); 2.503(124.1); 2.329(0.8); 1.288(7.5); 1.270(16.0); 1.252(7.2); 1.236(0.5); 0.000(2.0) |
| 74 | 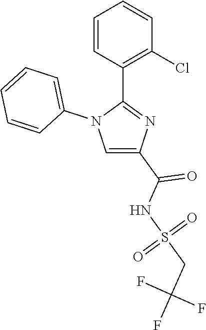 [SMILES: O=C(NS(=O)(=O)CC(F)(F)F)c1cn(-c2ccccc2)c(-c2ccccc2Cl)n1] | Example 74: ¹H-NMR(400.0 MHz, d₆-DMSO): d = 8.524(12.9); 7.690(3.9); 7.675(3.9); 7.672(4.3); 7.528(1.1); 7.523(1.2); 7.517(0.5); 7.511(1.6); 7.506 (3.0); 7.503(3.5); 7.490(5.0); 7.486(4.9); 7.470(8.9); 7.453(3.8); 7.448(5.6); 7.444(4.4); 7.438(1.6); 7.426(3.2); 7.415(7.2); 7.408(5.4); 7.401(13.2); 7.397 (16.0); 7.391(3.7); 7.385(2.1); 7.374(0.6); 7.369(0.5); 7.311(0.4); 7.295(7.6); 7.289(5.9); 7.276(6.0); 7.272(5.0); 4.859(2.3); 4.835(6.5); 4.810(6.8); 4.786 (2.6); 4.706(0.4); 4.656(0.5); 4.632(0.5); 4.595(0.5); 4.564(0.5); 4.491(0.6); 4.474(0.6); 4.459(0.6); 4.436(0.6); 4.427(0.6); 4.405(0.6); 4.351(0.7); 4.317 (0.7); 4.300(0.7); 4.282(0.7); 4.259(0.7); 4.229(0.7); 4.202(0.7); 4.058(0.6); 2.997(1.2); 2.712(0.6); 2.672(0.4); 2.567(0.4); 2.562(0.4); 2.542(125.9); 2.525(1.0); 2.521(1.1); 2.512(16.5); 2.507(34.5); 2.503(45.8); 2.498(33.8); 2.494(16.7); 2.368(0.5); 1.235(0.4); 0.000(2.2) |
| 75 | 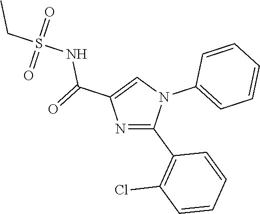 [SMILES: CCS(=O)(=O)NC(=O)c1cn(-c2ccccc2)c(-c2ccccc2Cl)n1] | Example 75: ¹H-NMR(400.0 MHz, d₆-DMSO): d = 8.489(13.3); 7.664(1.7); 7.659(3.3); 7.647(1.5); 7.644(2.4); 7.640(3.5); 7.514(0.7); 7.510(0.8); 7.500 (0.9); 7.491(2.8); 7.477(3.5); 7.472(3.4); 7.458(8.3); 7.440(4.1); 7.436(3.7); 7.429(1.0); 7.423(1.7); 7.419(2.0); 7.406(4.7); 7.388(9.4); 7.378(2.1); 7.374 (1.3); 7.361(0.4); 7.357(0.4); 7.269(5.5); 7.263(4.5); 7.256(2.1); 7.249(4.6); 7.245(3.8); 3.533(2.3); 3.515(6.9); 3.496(7.1); 3.478(2.7); 3.358(2.5); 3.169 (0.4); 2.998(0.7); 2.543(62.4); 2.526(0.5); 2.521(0.6); 2.512(9.3); 2.508 (19.2); 2.503(25.6); 2.499(19.0); 2.495(9.6); 1.293(7.3); 1.275(16.0); 1.257 (7.1); 0.000(0.8) |
| 76 | 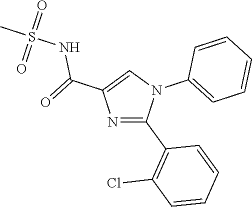 [SMILES: CS(=O)(=O)NC(=O)c1cn(-c2ccccc2)c(-c2ccccc2Cl)n1] | Example 76: ¹H-NMR(400.0 MHz, d₆-DMSO): d = 8.489(16.0); 7.657(2.1); 7.652(4.2); 7.640(1.9); 7.637(3.2); 7.633(4.5); 7.515(0.8); 7.511(1.0); 7.501 (1.1); 7.491(3.5); 7.478(4.3); 7.473(4.3); 7.458(10.8); 7.441(5.3); 7.436(4.6); 7.430(1.4); 7.424(2.2); 7.420(2.6); 7.407(6.0); 7.389(12.6); 7.378(2.8); 7.374 (1.9); 7.362(0.6); 7.358(0.5); 7.266(6.7); 7.260(6.0); 7.246(5.9); 7.242(5.0); 3.534(0.6); 3.362(37.7); 3.211(0.5); 3.186(0.6); 2.997(1.0); 2.712(0.4); 2.542(88.2); 2.525(0.7); 2.507(22.1); 2.503(29.7); 2.498(22.7); 2.368(0.5); 0.000(1.9) |
| 77 | 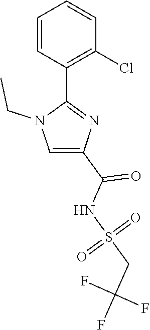 [SMILES: CCn1cc(C(=O)NS(=O)(=O)CC(F)(F)F)nc1-c1ccccc1Cl] | Example 77: ¹H-NMR(400.0 MHz, d₆-DMSO): d = 8.328(10.5); 7.735(2.0); 7.732(2.3); 7.723(2.6); 7.719(3.5); 7.715(5.2); 7.712(5.1); 7.705(3.5); 7.701 (4.0); 7.695(2.7); 7.691(2.4); 7.676(3.3); 7.672(2.8); 7.656(1.6); 7.652(1.4); 7.587(2.3); 7.584(2.5); 7.569(3.4); 7.565(3.4); 7.550(1.4); 7.547(1.5); 4.746 (0.3); 4.719(0.4); 4.716(0.3); 4.598(1.9); 4.573(5.2); 4.547(5.4); 4.522(2.1); 4.486(0.5); 4.472(0.5); 4.462(0.5); 4.439(0.5); 4.432(0.5); 4.421(0.5); 4.397 (0.5); 4.372(0.5); 4.343(0.5); 4.320(0.5); 4.289(0.5); 4.269(0.5); 4.244(0.5); 4.210(0.5); 4.195(0.5); 4.192(0.5); 4.189(0.5); 4.163(0.4); 4.144(0.4); 3.894 (1.9); 3.876(6.1); 3.858(6.2); 3.840(2.1); 2.997(0.4); 2.542(51.0); 2.525(0.6); 2.511(17.6); 2.507(36.8); 2.503(49.4); 2.498(37.6); 2.329(0.4); 1.289(7.3); 1.271(16.0); 1.253(7.4); 1.235(0.4); 0.000(3.7) |
| 78 | 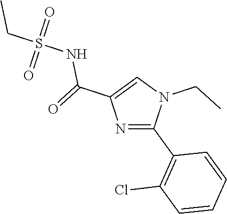 [SMILES: CCn1cc(C(=O)NS(=O)(=O)CC)nc1-c1ccccc1Cl] | Example 78: ¹H-NMR(400.0 MHz, d₆-DMSO): d = 8.292(10.8); 7.679(1.9); 7.677(2.0); 7.659(3.8); 7.657(3.6); 7.625(1.6); 7.620(1.9); 7.607(2.1); 7.603 (2.7); 7.587(2.5); 7.582(3.5); 7.567(4.0); 7.563(2.5); 7.534(2.5); 7.531(2.6); 7.516(2.4); 7.497(1.0); 7.494(0.9); 3.838(1.8); 3.820(5.7); 3.802(5.8); 3.784 (1.9); 3.641(0.4); 3.493(3.3); 3.474(8.0); 3.456(8.7); 3.437(5.5); 3.362(11.7); 2.996(0.8); 2.676(0.6); 2.672(0.8); 2.667(0.6); 2.542(50.0); 2.525(2.0); 2.520 (3.1); 2.511(47.8); 2.507(98.5); 2.502(130.2); 2.498(94.1); 2.493(45.6); 2.338(0.3); 2.334(0.7); 2.329(0.9); 2.325(0.6); 2.320(0.3); 1.264(7.0); 1.258 (7.5); 1.246(16.0); 1.240(15.9); 1.227(7.2); 1.222(7.1); 0.000(7.3) |
| 79 | 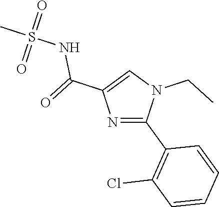 [SMILES: CCn1cc(C(=O)NS(C)(=O)=O)nc1-c1ccccc1Cl] | Example 79: ¹H-NMR(400.0 MHz, d₆-DMSO): d = 8.291(10.0); 7.678(2.3); 7.660(4.3); 7.658(4.2); 7.626(1.7); 7.621(2.0); 7.609(2.3); 7.604(2.9); 7.589 (1.2); 7.584(1.9); 7.580(1.8); 7.575(2.0); 7.560(4.5); 7.556(3.1); 7.535(2.8); 7.532(2.8); 7.515(2.8); 7.498(1.0); 7.495(0.9); 3.838(2.0); 3.820(6.3); 3.802 (6.4); 3.784(2.2); 3.640(0.4); 3.630(0.5); 3.489(1.7); 3.348(19.2); 3.318 (41.3); 3.143(0.9); 3.060(0.4); 3.048(0.4); 2.996(0.3); 2.676(1.1); 2.671(1.5); 2.667(1.2); 2.542(7.6); 2.525(3.4); 2.511(88.2); 2.507(178.9); 2.502(234.7); 2.498(171.5); 2.494(85.4); 2.333(1.1); 2.329(1.5); 2.325(1.2); 1.255(7.2); 1.237(16.0); 1.219(7.1); 0.008(0.4); 0.000(12.6); −0.008(0.5) |
| 80 | 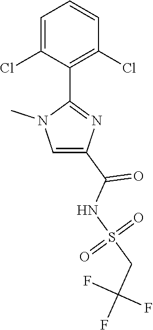 [SMILES: Cn1cc(C(=O)NS(=O)(=O)CC(F)(F)F)nc1-c1c(Cl)cccc1Cl] | Example 80: ¹H-NMR(400.0 MHz, d₆-DMSO): d = 8.305(5.8); 7.735(2.0); 7.728(2.9); 7.712(7.9); 7.691(3.9); 7.676(2.0); 7.667(1.2); 7.652(0.8); 4.771 (1.0); 4.746(2.9); 4.722(3.0); 4.697(1.1); 3.537(16.0); 2.997(0.4); 2.542 (38.8); 2.525(0.3); 2.512(6.9); 2.507(14.4); 2.503(19.3); 2.499(14.4); 2.494 (7.3); 0.000(1.4) |
| 81 | 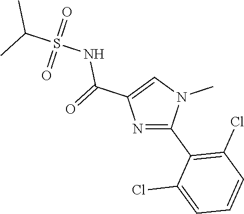 [SMILES: CC(C)S(=O)(=O)NC(=O)c1cn(C)c(-c2c(Cl)cccc2Cl)n1] | Example 81: ¹H-NMR(400.0 MHz, d₆-DMSO): d = 8.255(5.4); 7.708(2.0); 7.703(2.7); 7.685(7.7); 7.657(2.9); 7.641(1.7); 7.634(1.3); 7.617(0.8); 3.805 (0.5); 3.789(1.2); 3.772(1.7); 3.755(1.3); 3.737(0.6); 3.491(16.0); 3.337 (23.0); 2.996(0.5); 2.671(0.6); 2.542(28.0); 2.502(90.1); 2.329(0.6); 1.313 (15.7); 1.295(15.7); 0.000(3.5) |
| 82 | 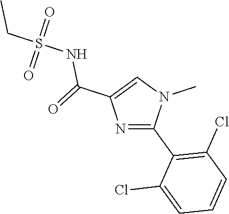 [SMILES: CCS(=O)(=O)NC(=O)c1cn(C)c(-c2c(Cl)cccc2Cl)n1] | Example 82: ¹H-NMR(400.0 MHz, d₆-DMSO): d = 8.255(5.7); 7.709(2.1); 7.703(2.9); 7.686(7.0); 7.658(3.4); 7.642(1.9); 7.634(1.3); 7.618(0.9); 3.492 (16.0); 3.481(4.0); 3.462(3.9); 3.444(1.5); 3.336(4.9); 2.996(0.5); 2.671(0.4); 2.542(31.3); 2.525(0.8); 2.511(21.2); 2.507(43.4); 2.502(57.3); 2.498(42.1); 2.493(21.0); 2.329(0.4); 1.266(3.9); 1.247(8.7); 1.229(3.8); 0.000(4.2) |
| 83 | 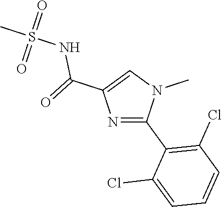 [SMILES: Cn1cc(C(=O)NS(C)(=O)=O)nc1-c1c(Cl)cccc1Cl] | Example 83: ¹H-NMR(400.0 MHz, d₆-DMSO): d = 8.245(3.1); 7.709(2.2); 7.703(3.1); 7.687(6.6); 7.685(7.2); 7.658(3.6); 7.642(2.0); 7.634(1.4); 7.618 (1.0); 3.492(16.0); 3.336(87.9); 2.676(1.2); 2.671(1.7); 2.667(1.2); 2.541 (3.2); 2.525(4.4); 2.520(7.0); 2.511(94.8); 2.507(193.3); 2.502(254.2); 2.498 (185.8); 2.493(92.1); 2.333(1.2); 2.329(1.6); 2.324(1.2); 1.236(0.8); 0.008 (0.6); 0.000(17.1); −0.009(0.6) |
| 84 | 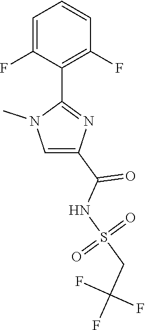 [SMILES: Cn1cc(C(=O)NS(=O)(=O)CC(F)(F)F)nc1-c1c(F)cccc1F] | Example 84: ¹H-NMR(400.0 MHz, d₆-DMSO): d = 19.993(0.6); 8.217(0.9); 7.744(1.1); 7.727(1.8); 7.707(1.3); 7.377(3.2); 7.357(5.4); 7.336(3.0); 7.204 (0.6); 7.076(0.6); 6.948(0.5); 4.658(1.1); 3.604(16.0); 3.363(968.4); 2.676 (5.2); 2.671(7.2); 2.667(5.5); 2.542(48.1); 2.525(17.7); 2.511(403.4); 2.507 (827.9); 2.502(1097.1); 2.498(816.1); 2.494(413.3); 2.334(5.0); 2.329(7.0); 2.325(5.3); 2.290(0.5); 1.298(0.7); 1.258(1.1); 1.235(3.3); 0.854(0.5); 0.008 (1.4); 0.000(39.8) |
| 85 | 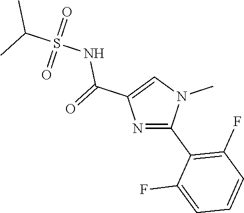 [SMILES: CC(C)S(=O)(=O)NC(=O)c1cn(C)c(-c2c(F)cccc2F)n1] | Example 85: ¹H-NMR(400.0 MHz, d₆-DMSO): d = 8.259(3.8); 7.748(0.3); 7.732(0.8); 7.727(0.7); 7.710(1.5); 7.694(0.8); 7.689(0.9); 7.672(0.4); 7.371 (0.4); 7.364(2.6); 7.344(3.8); 7.323(2.2); 7.316(0.5); 3.799(0.5); 3.781(1.2); 3.764(1.8); 3.747(1.3); 3.730(0.5); 3.589(13.6); 3.348(456.9); 2.996(0.4); 2.676(1.3); 2.672(1.9); 2.668(1.4); 2.542(6.6); 2.525(4.5); 2.520(6.8); 2.512 (102.7); 2.507(213.2); 2.503(282.9); 2.498(205.1); 2.494(99.4); 2.339(0.6); 2.334(1.3); 2.330(1.8); 2.325(1.3); 1.310(16.0); 1.292(15.8); 1.235(0.6); 0.000(1.0) |
| 86 | 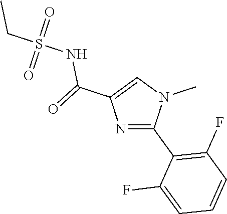 [SMILES: CCS(=O)(=O)NC(=O)c1cn(C)c(-c2c(F)cccc2F)n1] | Example 86: ¹H-NMR(400.0 MHz, d₆-DMSO): d = 8.263(3.8); 7.754(0.4); 7.733(0.9); 7.717(1.6); 7.696(1.1); 7.679(0.5); 7.370(2.8); 7.350(4.4); 7.329 (2.4); 3.595(16.0); 3.493(3.3); 3.475(6.2); 3.457(7.6); 3.366(936.7); 2.679 (2.5); 2.549(5.3); 2.514(291.7); 2.510(372.4); 2.505(287.3); 2.336(2.4); 1.265(4.6); 1.247(9.8); 1.228(4.4); 0.006(0.4) |
| 87 | 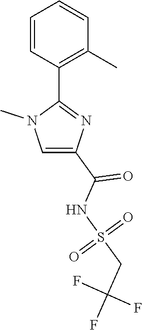 [SMILES: Cc1ccccc1-c1nc(C(=O)NS(=O)(=O)CC(F)(F)F)cn1C] | Example 87: ¹H-NMR(400.0 MHz, d₆-DMSO): d = 20.003(0.3); 8.148(0.7); 7.558(0.6); 7.541(2.8); 7.522(2.9); 7.456(2.0); 7.436(1.5); 7.413(1.3); 7.395 (1.8); 7.376(0.8); 4.401(0.6); 4.375(1.8); 4.349(1.8); 4.323(0.7); 3.559(13.0); 3.372(663.8); 2.995(0.8); 2.712(0.5); 2.676(3.3); 2.672(4.5); 2.667(3.3); 2.542(73.0); 2.525(10.7); 2.520(16.3); 2.511(245.1); 2.507(503.9); 2.503 (663.7); 2.498(481.7); 2.494(234.0); 2.368(0.4); 2.334(3.0); 2.329(4.2); 2.325(3.1); 2.290(0.3); 2.188(16.0); 1.297(0.4); 1.258(0.5); 1.235(1.5); 0.008(0.9); 0.000(27.7); −0.009(0.7) |
| 88 | 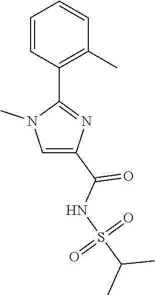 [SMILES: Cc1ccccc1-c1nc(C(=O)NS(=O)(=O)C(C)C)cn1C] | Example 88: ¹H-NMR(400.0 MHz, d₆-DMSO): d = 20.006(1.0); 8.317(0.7); 8.141(1.0); 7.432(1.5); 7.413(1.6); 7.388(2.5); 7.366(2.6); 7.335(1.4); 7.319 (1.6); 3.779(1.1); 3.763(1.3); 3.745(1.1); 3.502(16.0); 3.342(3315.1); 2.676 (11.6); 2.671(16.0); 2.667(11.8); 2.542(25.2); 2.525(39.9); 2.511(898.0); 2.507(1823.7); 2.502(2396.2); 2.498(1749.3); 2.494(857.3); 2.334(11.3); 2.329(15.3); 2.325(11.3); 2.290(1.3); 2.182(14.6); 1.304(10.6); 1.287(10.6); 1.258(2.0); 1.235(3.5); 0.008(3.5); 0.000(102.3); −0.009(3.1) |
| 89 | 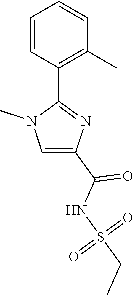 [SMILES: CCS(=O)(=O)NC(=O)c1cn(C)c(-c2ccccc2C)n1] | Example 89: ¹H-NMR(400.0 MHz, d₆-DMSO): d = 20.007(1.5); 7.433(1.1); 7.415(1.2); 7.390(1.9); 7.321(1.2); 3.503(9.9); 3.341(3867.6); 2.672(16.0); 2.542(22.6); 2.502(2379.1); 2.329(14.9); 2.180(9.7); 1.299(1.2); 1.253(2.7); 1.235(7.7); 1.216(2.2); 0.000(72.6) |
| 90 | 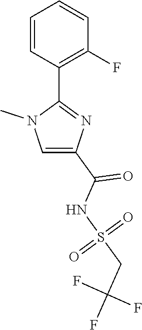 [SMILES: Cn1cc(C(=O)NS(=O)(=O)CC(F)(F)F)nc1-c1ccccc1F] | Example 90: ¹H-NMR(400.0 MHz, d₆-DMSO): d = 20.009(0.3); 8.184(1.6); 7.710(2.1); 7.692(4.3); 7.674(3.1); 7.659(1.1); 7.492(1.6); 7.468(2.4); 7.439 (2.3); 7.418(3.4); 7.401(1.6); 7.204(0.5); 7.076(0.5); 6.949(0.5); 4.564(1.9); 4.538(1.9); 3.654(16.0); 3.383(676.8); 2.996(1.4); 2.712(0.7); 2.676(3.8); 2.672(5.2); 2.667(3.9); 2.542(137.1); 2.525(12.3); 2.520(18.9); 2.511(283.8); 2.507(586.4); 2.502(777.2); 2.498(570.8); 2.494(283.3); 2.368(0.7); 2.334 (3.6); 2.329(5.0); 2.325(3.7); 2.290(0.4); 1.298(0.4); 1.258(0.6); 1.235(1.4); 0.008(1.2); 0.000(35.7); −0.008(1.1) |
| 91 | 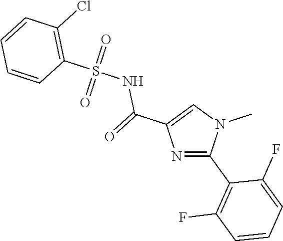 [SMILES: Cn1cc(C(=O)NS(=O)(=O)c2ccccc2Cl)nc1-c1c(F)cccc1F] | Example 91: ¹H-NMR(400.0 MHz, d₆-DMSO): d = 8.245(4.8); 8.161(2.0); 8.158(2.2); 8.142(2.2); 8.138(2.3); 7.745(0.4); 7.729(1.0); 7.723(1.3); 7.719 (0.8); 7.707(2.1); 7.703(2.3); 7.699(1.7); 7.690(1.2); 7.685(2.8); 7.681(2.1); 7.669(3.0); 7.666(3.5); 7.650(1.3); 7.646(1.0); 7.629(1.5); 7.625(1.3); 7.609 (1.9); 7.591(1.0); 7.587(0.9); 7.364(0.5); 7.357(3.1); 7.337(4.5); 7.316(2.6); 7.309(0.5); 3.750(0.4); 3.740(0.3); 3.725(0.4); 3.714(0.4); 3.705(0.4); 3.630 (0.5); 3.620(0.5); 3.576(16.0); 3.518(0.6); 3.484(0.6); 3.414(0.5); 3.397(0.6); 3.366(0.5); 3.292(0.4); 2.676(0.4); 2.671(0.6); 2.667(0.4); 2.525(1.0); 2.520 (1.8); 2.511(32.9); 2.507(68.6); 2.502(91.9); 2.498(68.9); 2.493(35.1); 2.334 (0.5); 2.329(0.7); 2.324(0.5); 2.075(0.4); 0.008(0.4); 0.000(13.1); −0.009(0.5) |
| 92 | 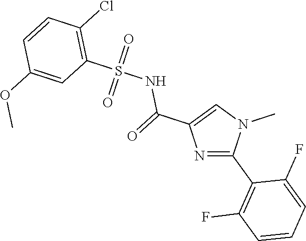 [SMILES: COc1ccc(Cl)c(S(=O)(=O)NC(=O)c2cn(C)c(-c3c(F)cccc3F)n2)c1] | Example 92: ¹H-NMR(400.0 MHz, d₆-DMSO): d = 8.242(3.4); 7.730(0.6); 7.726(0.6); 7.709(1.2); 7.692(0.6); 7.688(0.7); 7.605(2.7); 7.598(2.9); 7.573 (2.3); 7.551(2.7); 7.358(2.0); 7.338(3.2); 7.317(1.8); 7.292(1.4); 7.284(1.3); 7.270(1.2); 7.262(1.2); 3.854(16.0); 3.813(0.4); 3.753(0.5); 3.670(0.6); 3.650 (0.6); 3.578(11.6); 3.399(0.6); 2.675(0.3); 2.671(0.5); 2.667(0.4); 2.524(0.8); 2.507(52.7); 2.502(70.0); 2.498(53.3); 2.329(0.5); 2.325(0.4); 0.008(2.1); 0.000(59.2); −0.008(2.8) |
| 93 | 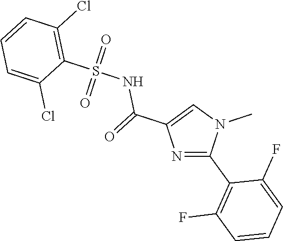 [SMILES: Cn1cc(C(=O)NS(=O)(=O)c2c(Cl)cccc2Cl)nc1-c1c(F)cccc1F] | Example 93: ¹H-NMR(400.0 MHz, d₆-DMSO): d = 8.232(3.7); 7.756(0.4); 7.740(0.9); 7.735(0.9); 7.718(1.7); 7.702(0.9); 7.697(1.0); 7.680(0.5); 7.656 (2.5); 7.652(3.1); 7.634(7.5); 7.595(2.8); 7.578(1.8); 7.572(1.4); 7.555(1.0); 7.372(0.7); 7.365(3.0); 7.345(4.7); 7.325(2.6); 4.022(0.4); 4.015(0.4); 3.995 (0.4); 3.779(0.7); 3.763(0.8); 3.729(0.7); 3.681(0.7); 3.588(16.0); 3.476(0.4); 3.438(0.4); 3.409(0.4); 3.186(0.4); 2.676(0.5); 2.671(0.7); 2.667(0.5); 2.511 (41.4); 2.507(80.6); 2.502(105.2); 2.498(78.3); 2.333(0.6); 2.329(0.7); 2.325 (0.6); 0.146(0.5); 0.008(5.6); 0.000(116.9); −0.008(5.1); −0.150(0.6) |
| 94 | 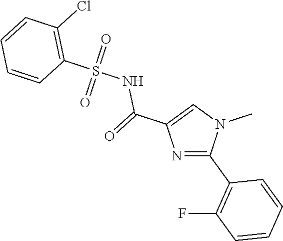 [SMILES: Cn1cc(C(=O)NS(=O)(=O)c2ccccc2Cl)nc1-c1ccccc1F] | Example 94: ¹H-NMR(400.0 MHz, d₆-DMSO): d = 8.317(0.4); 8.169(3.5); 8.150(2.7); 8.147(2.9); 8.130(2.8); 8.127(2.9); 7.694(0.6); 7.677(1.9); 7.660 (2.8); 7.656(3.0); 7.644(5.7); 7.623(5.7); 7.604(4.4); 7.589(2.5); 7.572(1.2); 7.569(1.1); 7.447(1.8); 7.423(2.5); 7.401(3.7); 7.382(3.8); 7.363(1.9); 7.361 (1.8); 4.131(0.3); 4.125(0.3); 4.068(0.4); 3.996(0.4); 3.981(0.4); 3.937(0.4); 3.889(0.5); 3.878(0.5); 3.862(0.5); 3.853(0.5); 3.780(0.5); 3.751(0.4); 3.732 (0.5); 3.708(0.5); 3.695(0.5); 3.605(15.8); 3.602(16.0); 3.425(0.5); 3.185 (0.3); 2.676(0.6); 2.671(0.9); 2.667(0.7); 2.525(1.7); 2.520(2.8); 2.511(52.0); 2.507(107.7); 2.502(143.8); 2.498(106.8); 2.493(53.6); 2.433(0.4); 2.333 (0.8); 2.329(1.1); 2.325(0.8); 2.075(0.5); 0.008(1.7); 0.000(57.9); −0.009 (2.3) |
| 95 | 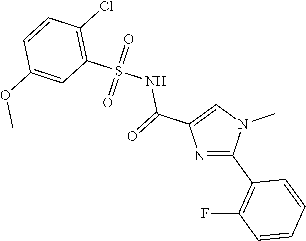 [SMILES: COc1ccc(Cl)c(S(=O)(=O)NC(=O)c2cn(C)c(-c3ccccc3F)n2)c1] | Example 95: ¹H-NMR(400.0 MHz, d₆-DMSO): d = 8.170(2.3); 7.660(0.4); 7.645(1.5); 7.627(2.7); 7.609(1.8); 7.602(2.9); 7.594(2.9); 7.549(2.0); 7.527 (2.3); 7.451(1.0); 7.427(1.4); 7.403(1.9); 7.384(2.0); 7.365(0.9); 7.264(1.2); 7.256(1.2); 7.242(1.1); 7.234(1.1); 4.027(0.4); 3.934(0.4); 3.896(0.4); 3.889 (0.4); 3.884(0.4); 3.848(16.0); 3.800(0.4); 3.784(0.4); 3.742(0.4); 3.721(0.4); 3.664(0.4); 3.607(9.1); 2.671(0.5); 2.667(0.4); 2.506(61.2); 2.502(82.1); 2.498(63.8); 2.333(0.4); 2.329(0.6); 2.325(0.5); 0.008(0.9); 0.000(28.4) |
| 96 | 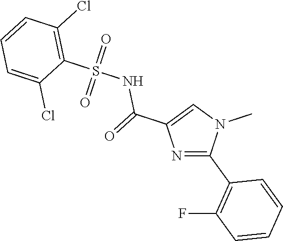 [SMILES: Cn1cc(C(=O)NS(=O)(=O)c2c(Cl)cccc2Cl)nc1-c1ccccc1F] | Example 96: ¹H-NMR(400.0 MHz, d₆-DMSO): d = 8.163(4.0); 7.683(0.8); 7.671(1.9); 7.666(1.9); 7.653(4.0); 7.648(2.0); 7.639(1.6); 7.634(2.4); 7.609 (3.6); 7.605(4.3); 7.587(9.5); 7.540(3.3); 7.523(2.3); 7.517(2.0); 7.500(1.3); 7.469(1.8); 7.457(0.4); 7.445(2.4); 7.419(2.8); 7.398(3.4); 7.381(1.6); 7.379 (1.6); 4.126(0.3); 3.848(2.9); 3.630(15.7); 3.628(16.0); 3.448(0.4); 2.676 (0.5); 2.671(0.8); 2.667(0.6); 2.525(1.4); 2.520(2.4); 2.511(43.6); 2.507 (90.9); 2.502(121.8); 2.498(91.8); 2.494(47.2); 2.334(0.7); 2.329(0.9); 2.325 (0.7); 2.075(0.4); 0.008(0.9); 0.000(29.3); −0.008(1.2) |
| 97 | 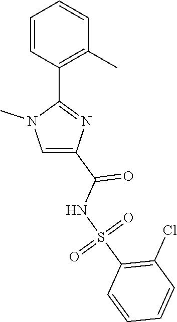 [SMILES: Cc1ccccc1-c1nc(C(=O)NS(=O)(=O)c2ccccc2Cl)cn1C] | Example 97: ¹H-NMR(400.0 MHz, d₆-DMSO): d = 8.124(1.7); 8.120(2.0); 8.108(3.3); 8.105(3.2); 7.629(0.3); 7.613(1.1); 7.609(1.1); 7.593(3.7); 7.589 (3.2); 7.574(0.9); 7.559(1.3); 7.554(1.0); 7.539(1.4); 7.523(0.7); 7.518(0.6); 7.490(0.7); 7.471(1.7); 7.453(1.5); 7.442(1.4); 7.424(2.2); 7.409(2.3); 7.390 (1.4); 7.362(1.3); 7.344(1.7); 7.325(0.7); 3.899(1.2); 3.730(0.5); 3.691(0.5); 3.515(16.0); 2.676(0.4); 2.671(0.6); 2.667(0.4); 2.524(1.4); 2.507(66.6); 2.502(86.5); 2.498(64.4); 2.333(0.5); 2.329(0.7); 2.171(14.2); 0.008(0.9); 0.000(26.1); −0.008(1.1) |
| 98 | 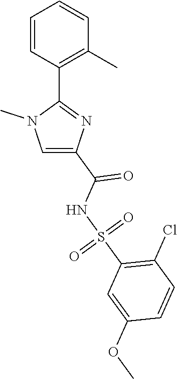 [SMILES: COc1ccc(Cl)c(S(=O)(=O)NC(=O)c2cn(C)c(-c3ccccc3C)n2)c1] | Example 98: ¹H-NMR(400.0 MHz, d₆-DMSO): d = 8.106(3.3); 7.593(2.7); 7.585(2.8); 7.498(0.5); 7.484(2.9); 7.462(3.6); 7.438(1.7); 7.414(1.7); 7.396 (1.0); 7.367(1.0); 7.349(1.3); 7.330(0.5); 7.187(1.3); 7.179(1.2); 7.165(1.1); 7.157(1.1); 4.104(0.4); 4.089(0.4); 4.008(0.7); 3.942(0.7); 3.920(0.7); 3.830 (16.0); 3.734(0.4); 3.697(0.3); 3.522(12.3); 2.671(0.4); 2.524(0.9); 2.510 (26.8); 2.506(54.6); 2.502(72.4); 2.498(54.3); 2.493(28.0); 2.329(0.6); 2.324 (0.4); 2.172(10.8); 0.008(0.6); 0.000(22.8); −0.008(1.0) |
| 99 | 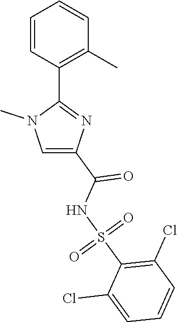 [SMILES: Cc1ccccc1-c1nc(C(=O)NS(=O)(=O)c2c(Cl)cccc2Cl)cn1C] | Example 99: ¹H-NMR(400.0 MHz, d₆-DMSO): d = 8.133(1.7); 8.109(1.9); 7.531(0.8); 7.521(3.3); 7.519(4.0); 7.512(2.7); 7.500(7.4); 7.494(2.6); 7.490 (2.5); 7.436(3.7); 7.419(2.4); 7.415(3.0); 7.397(1.3); 7.391(1.4); 7.372(1.9); 7.353(0.7); 3.795(2.8); 3.583(0.4); 3.546(16.0); 3.514(0.3); 2.676(0.4); 2.671 (0.5); 2.667(0.4); 2.525(1.1); 2.520(1.7); 2.511(29.3); 2.507(61.4); 2.502 (82.1); 2.498(60.2); 2.493(29.7); 2.333(0.5); 2.329(0.6); 2.324(0.4); 2.174 (14.4); 2.075(1.0); 0.000(6.7) |
| 100 | 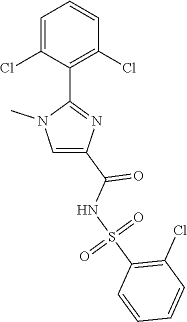 [SMILES: Cn1cc(C(=O)NS(=O)(=O)c2ccccc2Cl)nc1-c1c(Cl)cccc1Cl] | Example 100: ¹H-NMR(400.0 MHz, d₆-DMSO): d = 8.244(4.3); 8.166(1.6); 8.162(1.8); 8.146(1.8); 8.142(1.8); 7.724(0.4); 7.720(0.5); 7.702(3.2); 7.696 (3.2); 7.686(1.9); 7.678(7.8); 7.674(2.6); 7.670(3.0); 7.655(4.7); 7.639(2.1); 7.631(2.5); 7.627(1.2); 7.615(1.3); 7.614(1.3); 7.611(1.5); 7.607(1.2); 7.594 (0.8); 7.590(0.7); 3.670(0.3); 3.656(0.4); 3.623(0.4); 3.613(0.4); 3.602(0.4); 3.598(0.4); 3.593(0.4); 3.563(0.4); 3.542(0.4); 3.531(0.4); 3.521(0.4); 3.482 (16.0); 2.525(0.6); 2.520(0.9); 2.512(16.8); 2.507(35.5); 2.503(47.8); 2.498 (35.1); 2.494(17.4); 2.329(0.4); 0.000(6.7) |
| 101 | 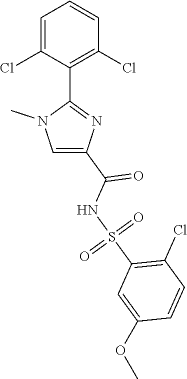 [SMILES: COc1ccc(Cl)c(S(=O)(=O)NC(=O)c2cn(C)c(-c3c(Cl)cccc3Cl)n2)c1] | Example 101: ¹H-NMR(400.0 MHz, d₆-DMSO): d = 8.233(1.9); 7.703(1.7); 7.697(2.4); 7.680(6.7); 7.656(3.0); 7.641(1.6); 7.632(1.0); 7.617(0.8); 7.607 (2.8); 7.599(3.0); 7.573(1.9); 7.551(2.2); 7.288(1.1); 7.281(1.1); 7.266(1.0); 7.259(1.0); 3.853(16.0); 3.668(0.4); 3.658(0.4); 3.608(0.3); 3.591(0.3); 3.533 (0.3); 3.484(13.3); 2.671(0.4); 2.507(45.6); 2.502(58.7); 2.498(44.1); 2.329 (0.4); 0.008(0.8); 0.000(20.0); −0.008(1.0) |
| 102 | 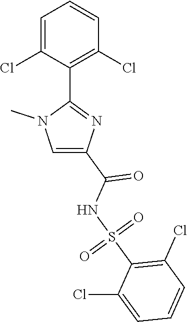 [SMILES: Cn1cc(C(=O)NS(=O)(=O)c2c(Cl)cccc2Cl)nc1-c1c(Cl)cccc1Cl] | Example 102: ¹H-NMR(400.0 MHz, d₆-DMSO): d = 8.227(2.8); 7.712(2.0); 7.705(2.9); 7.689(8.1); 7.667(3.8); 7.652(4.6); 7.643(1.6); 7.633(6.4); 7.591 (2.3); 7.574(1.5); 7.569(1.3); 7.552(0.8); 3.499(16.0); 2.676(0.4); 2.671(0.5); 2.667(0.4); 2.511(24.9); 2.507(49.0); 2.502(63.9); 2.498(47.3); 2.329(0.4); 0.008(1.0); 0.000(23.7); −0.008(0.8) |
| 103 | 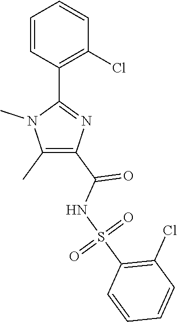 [SMILES: Cc1c(C(=O)NS(=O)(=O)c2ccccc2Cl)nc(-c2ccccc2Cl)n1C] | Example 103: ¹H-NMR(400.0 MHz, d₆-DMSO): d = 8.142(1.4); 8.138(1.5); 8.122(1.6); 8.119(1.6); 7.692(1.2); 7.690(1.2); 7.672(2.5); 7.670(2.5); 7.663 (1.1); 7.642(2.5); 7.638(3.0); 7.634(2.4); 7.625(1.5); 7.620(2.1); 7.605(1.8); 7.601(2.9); 7.586(2.7); 7.582(2.6); 7.565(0.6); 7.560(0.6); 7.552(1.6); 7.549 (1.6); 7.533(1.5); 7.515(0.6); 7.512(0.6); 3.787(1.1); 3.761(1.1); 3.744(1.1); 3.712(1.0); 3.627(0.7); 3.548(0.5); 3.374(15.0); 2.676(0.4); 2.672(0.5); 2.667 (0.4); 2.525(1.3); 2.520(2.0); 2.511(29.8); 2.507(61.6); 2.502(81.6); 2.498 (59.6); 2.493(29.2); 2.469(16.0); 2.334(0.4); 2.329(0.6); 2.325(0.4); 0.000 (5.0) |
| 104 | 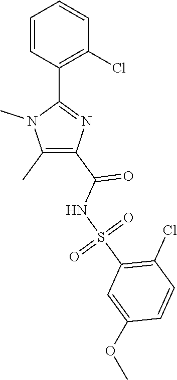 [SMILES: COc1ccc(Cl)c(S(=O)(=O)NC(=O)c2nc(-c3ccccc3Cl)n(C)c2C)c1] | Example 104: ¹H-NMR(400.0 MHz, d₆-DMSO): d = 7.696(1.0); 7.693(1.1); 7.675(2.1); 7.647(0.8); 7.642(1.0); 7.629(1.1); 7.625(1.5); 7.605(1.6); 7.599 (3.1); 7.591(4.7); 7.554(1.3); 7.551(1.4); 7.536(3.0); 7.514(2.4); 7.244(1.0); 7.236(1.0); 7.221(0.9); 7.214(0.8); 4.048(0.4); 4.041(0.4); 4.026(0.4); 4.001 (0.4); 3.948(0.4); 3.922(0.4); 3.847(16.0); 3.381(12.3); 2.671(0.4); 2.524 (1.1); 2.511(25.7); 2.507(52.1); 2.502(68.9); 2.498(51.9); 2.493(27.8); 2.487 (17.1); 2.333(0.4); 2.329(0.5); 2.324(0.4); 2.075(0.4); 0.000(3.9) |
| 105 | 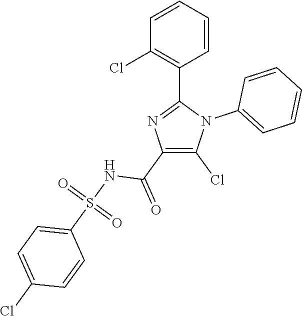 [SMILES: O=C(NS(=O)(=O)c1ccc(Cl)cc1)c1nc(-c2ccccc2Cl)n(-c2ccccc2)c1Cl] | Example 105: ¹H NMR (400 MHz, CDCl3) δ 8.14 (d, J =8.8 Hz, 2H), 7.54 (d, J =8.4 Hz, 2H), 7.41-7.23 (m, 7H), 7.12 (d, J =8.4 Hz, 2H). |
| 106 | 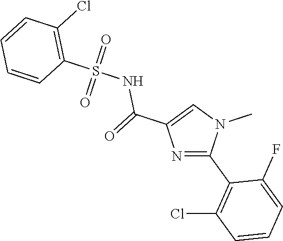 [SMILES: Cn1cc(C(=O)NS(=O)(=O)c2ccccc2Cl)nc1-c1c(F)cccc1Cl] | Example 106: ¹H-NMR(400.0 MHz, d₆-DMSO): d = 8.244(5.3); 8.165(1.7); 8.161(1.9); 8.145(1.9); 8.141(2.0); 7.724(0.5); 7.720(0.5); 7.710(0.8); 7.704 (1.5); 7.700(1.4); 7.695(1.0); 7.690(2.0); 7.687(2.2); 7.683(1.9); 7.672(3.1); 7.668(4.0); 7.653(1.9); 7.648(1.0); 7.630(1.3); 7.626(1.1); 7.610(1.7); 7.593 (0.9); 7.589(0.8); 7.566(2.7); 7.546(1.9); 7.482(1.2); 7.460(2.1); 7.439(1.1); 5.753(0.8); 3.568(0.4); 3.556(0.3); 3.527(16.0); 3.498(0.5); 3.478(0.3); 3.451 (0.3); 3.432(0.3); 3.347(0.4); 2.675(0.5); 2.671(0.6); 2.666(0.5); 2.524(1.3); 2.510(36.4); 2.506(73.4); 2.502(97.0); 2.497(73.0); 2.493(38.0); 2.333(0.5); 2.328(0.7); 2.324(0.5); 0.146(0.5); 0.008(4.0); 0.000(110.8); −0.008(5.1); −0.150(0.5) |
| 107 | 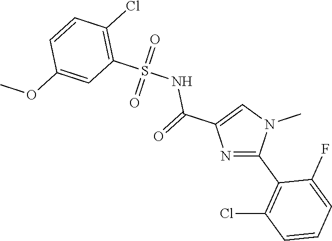 [SMILES: COc1ccc(Cl)c(S(=O)(=O)NC(=O)c2cn(C)c(-c3c(F)cccc3Cl)n2)c1] | Example 107: ¹H-NMR(400.0 MHz, d₆-DMSO): d = 8.239(3.2); 7.712(0.5); 7.696(0.7); 7.691(1.3); 7.676(1.3); 7.671(1.0); 7.655(0.9); 7.608(2.7); 7.600 (2.9); 7.573(2.5); 7.568(2.7); 7.551(3.2); 7.483(1.1); 7.461(1.9); 7.439(0.9); 7.292(1.3); 7.284(1.4); 7.270(1.2); 7.263(1.2); 3.854(16.0); 3.529(13.2); 2.675(0.3); 2.671(0.5); 2.666(0.4); 2.524(1.4); 2.506(58.3); 2.502(76.3); 2.497(58.2); 2.333(0.4); 2.328(0.5); 2.324(0.4); 2.073(1.9); 0.146(0.4); 0.008 (2.9); 0.000(73.3); −0.008(4.0); −0.150(0.4) |
| 108 | 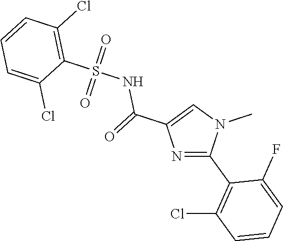 [SMILES: Cn1cc(C(=O)NS(=O)(=O)c2c(Cl)cccc2Cl)nc1-c1c(F)cccc1Cl] | Example 108: ¹H-NMR(400.0 MHz, d₆-DMSO): d = 8.234(5.0); 7.722(0.7); 7.706(0.7); 7.701(1.6); 7.686(1.6); 7.680(1.2); 7.665(1.2); 7.660(2.6); 7.655 (3.2); 7.637(7.2); 7.599(3.0); 7.582(2.0); 7.575(4.1); 7.555(2.1); 7.492(1.2); 7.490(1.2); 7.470(2.2); 7.449(1.1); 7.447(1.0); 3.541(16.0); 2.670(0.4); 2.524 (0.4); 2.519(0.9); 2.510(32.0); 2.506(68.6); 2.501(93.4); 2.497(70.7); 2.492 (36.9); 2.333(0.6); 2.328(0.8); 2.324(0.6); 2.073(0.6); 0.146(0.4); 0.008(2.8); 0.000(110.5); −0.009(5.4); −0.033(0.4); −0.150(0.6) |
| 109 | 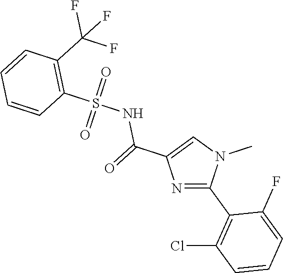 [SMILES: Cn1cc(C(=O)NS(=O)(=O)c2ccccc2C(F)(F)F)nc1-c1c(F)cccc1Cl] | Example 109: ¹H-NMR(400.0 MHz, d₆-DMSO): d = 8.353(1.7); 8.334(1.8); 8.231(5.2); 8.006(1.3); 7.987(2.0); 7.970(0.7); 7.954(1.6); 7.935(1.3); 7.919 (1.4); 7.900(1.5); 7.882(0.5); 7.718(0.7); 7.702(0.8); 7.697(1.6); 7.682(1.6); 7.676(1.2); 7.661(1.1); 7.572(2.7); 7.552(2.0); 7.487(1.2); 7.466(2.2); 7.445 (1.0); 3.533(16.0); 2.675(0.4); 2.671(0.6); 2.666(0.4); 2.524(1.1); 2.510 (37.9); 2.506(77.4); 2.502(102.6); 2.497(76.9); 2.493(39.5); 2.444(0.4); 2.333(0.5); 2.328(0.7); 2.324(0.6); 0.146(0.5); 0.008(3.8); 0.000(111.9); −0.009(4.7); −0.150(0.5) |
| 110 | 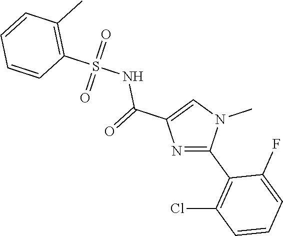 [SMILES: Cc1ccccc1S(=O)(=O)NC(=O)c1cn(C)c(-c2c(F)cccc2Cl)n1] | Example 110: ¹H-NMR(400.0 MHz, d₆-DMSO): d = 8.172(4.2); 8.030(1.8); 8.013(1.8); 8.010(1.9); 7.706(0.7); 7.691(0.8); 7.686(1.6); 7.670(1.7); 7.665 (1.2); 7.649(1.1); 7.585(0.7); 7.566(3.3); 7.564(3.3); 7.551(1.3); 7.543(2.2); 7.478(1.2); 7.476(1.2); 7.455(3.2); 7.435(2.7); 7.417(0.8); 7.400(1.9); 7.381 (1.6); 3.512(16.0); 3.333(0.5); 3.321(0.5); 3.302(0.5); 3.272(0.4); 2.890(0.4); 2.731(0.3); 2.675(0.4); 2.670(0.5); 2.666(0.4); 2.607(13.9); 2.524(1.6); 2.510(31.5); 2.506(62.4); 2.501(82.2); 2.497(61.5); 2.492(31.3); 2.332(0.4); 2.328(0.5); 2.324(0.4); 0.008(2.2); 0.000(62.9); −0.009(2.8) |
| 111 | 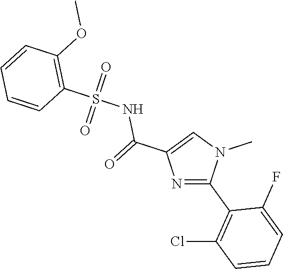 [SMILES: COc1ccccc1S(=O)(=O)NC(=O)c1cn(C)c(-c2c(F)cccc2Cl)n1] | Example 111: ¹H-NMR(400.0 MHz, d₆-DMSO): d = 8.217(3.6); 7.905(1.8); 7.901(1.9); 7.885(1.9); 7.881(1.9); 7.711(0.6); 7.690(1.8); 7.683(1.2); 7.675 (1.9); 7.669(2.3); 7.654(1.3); 7.647(1.2); 7.643(1.0); 7.570(2.6); 7.550(1.9); 7.486(1.2); 7.463(2.1); 7.442(1.0); 7.238(2.2); 7.217(2.0); 7.165(1.2); 7.146 (2.1); 7.127(1.1); 3.842(16.0); 3.519(14.2); 3.318(3.6); 2.670(0.7); 2.505 (79.3); 2.501(98.2); 2.497(77.9); 2.328(0.6); 2.085(0.5); 2.073(4.7); 0.000 (55.0) |
| 112 | 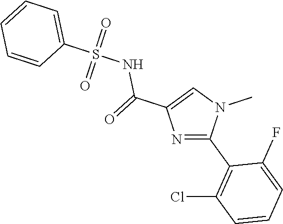 [SMILES: Cn1cc(C(=O)NS(=O)(=O)c2ccccc2)nc1-c1c(F)cccc1Cl] | Example 112: ¹H-NMR(400.0 MHz, d₆-DMSO): d = 8.160(6.0); 8.010(3.3); 7.992(4.0); 7.989(3.1); 7.726(0.7); 7.714(0.5); 7.707(2.5); 7.690(2.2); 7.686 (2.5); 7.670(1.7); 7.665(1.2); 7.647(3.3); 7.627(4.1); 7.614(0.8); 7.609(1.7); 7.563(2.7); 7.542(2.0); 7.478(1.2); 7.475(1.2); 7.455(2.1); 7.434(1.1); 7.432 (1.0); 5.753(9.2); 3.506(16.0); 3.327(1.0); 3.316(0.9); 3.301(0.8); 3.223(0.4); 3.186(0.4); 2.891(1.0); 2.764(0.6); 2.731(0.9); 2.675(0.4); 2.671(0.5); 2.666 (0.4); 2.524(1.0); 2.510(27.8); 2.506(56.8); 2.502(75.8); 2.497(56.8); 2.493 (28.9); 2.333(0.3); 2.328(0.5); 2.324(0.4); 0.008(1.9); 0.000(55.3); −0.008 (2.2) |
| 113 | 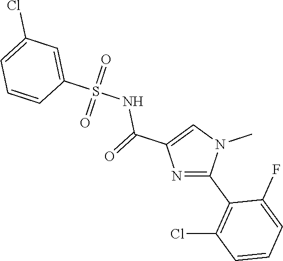 [SMILES: Cn1cc(C(=O)NS(=O)(=O)c2cccc(Cl)c2)nc1-c1c(F)cccc1Cl] | Example 113: ¹H-NMR(400.0 MHz, d₆-DMSO): d = 8.191(6.6); 8.002(1.7); 7.998(3.2); 7.993(2.2); 7.965(1.3); 7.963(1.7); 7.958(1.2); 7.946(1.6); 7.943 (1.8); 7.941(1.7); 7.939(1.4); 7.816(1.1); 7.814(1.3); 7.811(1.2); 7.808(1.2); 7.796(1.6); 7.793(1.7); 7.791(1.7); 7.788(1.5); 7.716(0.8); 7.696(3.0); 7.677 (4.1); 7.658(2.1); 7.571(2.7); 7.550(1.9); 7.486(1.2); 7.484(1.2); 7.463(2.0); 7.442(1.0); 7.440(1.0); 3.623(0.3); 3.554(0.3); 3.519(16.0); 3.474(0.3); 3.339 (0.3); 3.325(0.4); 2.675(0.5); 2.671(0.6); 2.666(0.5); 2.645(0.8); 2.524(1.3); 2.519(2.2); 2.511(32.9); 2.506(67.3); 2.502(89.1); 2.497(65.9); 2.493(32.6); 2.333(0.4); 2.329(0.6); 2.324(0.4); 2.074(12.9); 1.372(0.6); 0.008(2.3); 0.000(69.9); −0.009(2.7) |
| 114 | 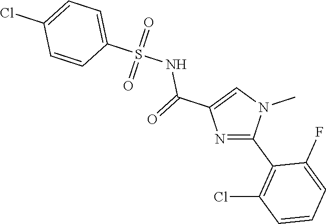 [SMILES: Cn1cc(C(=O)NS(=O)(=O)c2ccc(Cl)cc2)nc1-c1c(F)cccc1Cl] | Example 114: ¹H-NMR(400.0 MHz, d₆-DMSO): d = 8.174(6.3); 8.132(0.4); 8.015(0.6); 8.009(4.8); 8.004(1.8); 7.992(1.8); 7.987(5.8); 7.981(0.9); 7.733 (0.9); 7.727(5.8); 7.722(2.1); 7.710(2.4); 7.705(5.1); 7.699(1.0); 7.695(1.1); 7.690(1.7); 7.675(1.7); 7.669(1.3); 7.654(1.1); 7.566(2.7); 7.546(2.0); 7.481 (1.3); 7.479(1.2); 7.459(2.2); 7.438(1.0); 7.436(1.0); 3.618(0.4); 3.584(0.4); 3.569(0.4); 3.557(0.4); 3.513(16.0); 3.475(0.5); 3.458(0.5); 3.450(0.5); 3.421(0.6); 3.415(0.6); 3.398(0.6); 3.381(0.6); 3.371(0.6); 3.334(0.6); 3.325 (0.6); 3.298(0.5); 3.264(0.4); 3.244(0.4); 3.187(0.3); 2.675(0.5); 2.671(0.6); 2.667(0.5); 2.524(1.7); 2.506(62.1); 2.502(81.6); 2.497(61.5); 2.493(31.7); 2.468(0.4); 2.464(0.4); 2.333(0.4); 2.329(0.5); 2.324(0.4); 2.074(8.7); 1.372 (0.5); 0.008(1.9); 0.000(55.1); −0.008(2.8) |
| 115 | 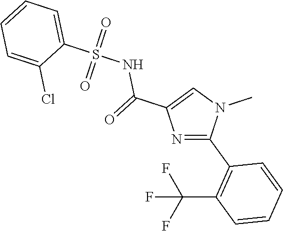 [SMILES: Cn1cc(C(=O)NS(=O)(=O)c2ccccc2Cl)nc1-c1ccccc1C(F)(F)F] | Example 115: ¹H-NMR(400.0 MHz, d₆-DMSO): d = 8.314(1.1); 8.167(12.2); 8.154(11.7); 8.133(10.7); 7.954(7.7); 7.936(10.0); 7.933(10.1); 7.858(2.5); 7.841(8.6); 7.826(15.6); 7.822(16.0); 7.806(8.4); 7.788(2.6); 7.691(13.4); 7.671(13.2); 7.658(15.1); 7.641(5.2); 7.615(5.8); 7.596(8.5); 7.579(3.9); 4.604(0.3); 4.567(0.3); 4.552(0.3); 4.530(0.4); 4.526(0.4); 4.486(0.4); 4.473 (0.4); 4.445(0.4); 4.383(0.5); 4.364(0.5); 4.320(0.5); 4.305(0.6); 4.195(0.7); 4.182(0.7); 4.122(0.8); 4.045(1.8); 3.967(1.1); 3.949(1.1); 3.907(1.2); 3.880 (1.2); 3.836(1.3); 3.806(1.3); 3.798(1.4); 3.788(1.4); 3.768(1.4); 3.756(1.4); 3.736(1.5); 3.721(1.4); 3.707(1.4); 3.681(1.5); 3.647(1.4); 3.624(1.7); 3.590 (1.4); 3.568(1.5); 3.509(1.3); 3.449(77.6); 3.415(1.9); 3.404(2.1); 3.356(1.1); 3.317(0.9); 3.269(1.2); 3.244(0.8); 3.228(0.7); 3.216(0.7); 3.205(0.7); 3.189 (0.7); 3.182(0.7); 3.149(0.6); 3.119(0.5); 3.109(0.5); 3.095(0.5); 3.055(0.5); 3.022(0.5); 3.003(0.5); 2.957(0.4); 2.925(0.3); 2.761(0.9); 2.671(3.4); 2.502 (532.9); 2.410(1.4); 2.328(3.9); 2.300(0.5); 2.269(0.5); 2.207(0.4); 2.073 |
| | | (0.5); 1.507(2.2); 1.106(0.4); 0.146(1.8); 0.000(373.2); −0.079(0.4); −0.088 |
| | | (0.4); −0.105(0.4); −0.150(2.0) |
| 116 | 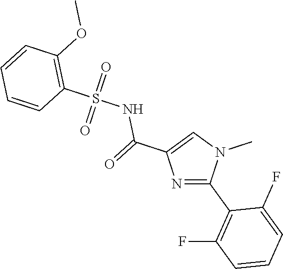 [SMILES: COc1ccccc1S(=O)(=O)NC(=O)c1cn(C)c(-c2c(F)cccc2F)n1] | Example 116: ¹H-NMR(400.0 MHz, d₆-DMSO): d = 8.219(4.8); 7.905(1.5); 7.901(1.7); 7.885(1.7); 7.881(1.8); 7.726(0.7); 7.722(0.6); 7.709(0.5); 7.705 (1.3); 7.687(1.3); 7.683(1.4); 7.666(1.5); 7.662(1.2); 7.647(0.9); 7.643(0.9); 7.364(0.4); 7.357(2.2); 7.336(3.3); 7.316(1.9); 7.309(0.4); 7.238(2.0); 7.217 (1.8); 7.165(1.1); 7.163(1.1); 7.145(1.9); 7.127(1.0); 7.125(1.0); 3.847(16.0); 3.571(11.1); 3.324(0.7); 2.524(0.5); 2.511(11.6); 2.506(24.1); 2.502(32.2); 2.497(24.1); 2.493(12.2); 2.073(8.5); 0.000(3.6) |
| 117 | 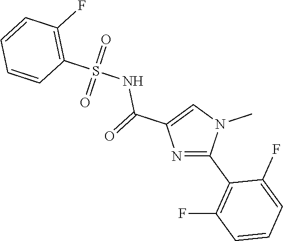 [SMILES: Cn1cc(C(=O)NS(=O)(=O)c2ccccc2F)nc1-c1c(F)cccc1F] | Example 117: ¹H-NMR(400.0 MHz, d₆-DMSO): d = 8.224(6.6); 8.000(0.8); 7.995(1.0); 7.979(1.6); 7.976(1.8); 7.961(1.0); 7.957(1.0); 7.789(0.5); 7.785 (0.5); 7.777(0.5); 7.770(1.1); 7.766(0.9); 7.758(0.9); 7.752(1.0); 7.746(1.1); 7.737(0.7); 7.732(0.7); 7.729(1.1); 7.724(0.9); 7.712(0.7); 7.708(1.8); 7.691 (0.9); 7.687(1.1); 7.670(0.5); 7.463(3.3); 7.444(3.4); 7.437(1.7); 7.427(1.4); 7.425(1.4); 7.416(1.2); 7.362(0.5); 7.355(3.2); 7.335(4.5); 7.315(2.7); 7.308 (0.6); 3.573(16.0); 3.530(0.3); 3.393(0.3); 2.671(0.4); 2.666(0.3); 2.524(0.7); 2.519(1.3); 2.511(25.1); 2.506(52.2); 2.502(70.0); 2.497(52.1); 2.493(26.2); 2.333(0.4); 2.329(0.5); 2.324(0.4); 0.000(7.4) |
| 118 | 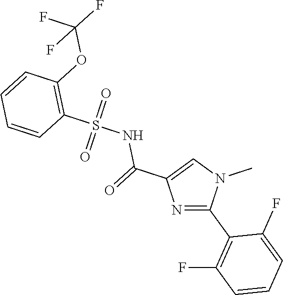 [SMILES: Cn1cc(C(=O)NS(=O)(=O)c2ccccc2OC(F)(F)F)nc1-c1c(F)cccc1F] | Example 118: ¹H-NMR(400.0 MHz, d₆-DMSO): d = 8.213(6.3); 8.130(2.1); 8.126(2.2); 8.110(2.4); 8.106(2.4); 7.849(0.9); 7.845(0.9); 7.828(1.8); 7.826 (1.8); 7.809(1.4); 7.805(1.3); 7.748(0.4); 7.732(0.9); 7.727(0.9); 7.710(1.8); 7.693(0.9); 7.689(1.1); 7.673(0.5); 7.638(1.6); 7.619(2.6); 7.602(1.2); 7.600 (1.3); 7.586(1.6); 7.565(1.4); 7.365(0.6); 7.358(3.1); 7.338(4.7); 7.317(2.7); 7.310(0.6); 3.579(16.0); 2.671(0.4); 2.524(0.5); 2.511(24.0); 2.506(49.3); 2.502(65.6); 2.497(48.7); 2.493(24.5); 2.333(0.4); 2.328(0.5); 2.324(0.4); 2.074(9.2); 0.008(0.7); 0.000(25.6); −0.008(1.1) |
| 119 | 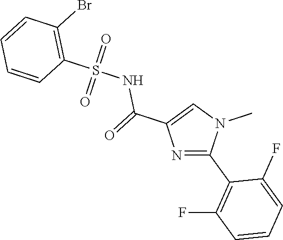 [SMILES: Cn1cc(C(=O)NS(=O)(=O)c2ccccc2Br)nc1-c1c(F)cccc1F] | Example 119: ¹H-NMR(400.0 MHz, d₆-DMSO): d = 8.312(0.4); 8.249(3.0); 8.190(2.0); 8.185(2.1); 8.170(2.1); 8.166(2.2); 7.848(1.8); 7.845(1.8); 7.829 (2.1); 7.826(2.2); 7.743(0.4); 7.726(0.9); 7.722(0.9); 7.705(1.7); 7.688(0.9); 7.684(1.1); 7.667(1.3); 7.651(1.9); 7.648(1.9); 7.632(1.4); 7.629(1.3); 7.609 (1.2); 7.605(1.3); 7.590(1.6); 7.586(1.6); 7.571(0.6); 7.567(0.6); 7.360(0.5); 7.353(3.1); 7.333(4.5); 7.312(2.6); 7.305(0.6); 3.802(0.4); 3.752(0.5); 3.577 (16.0); 3.517(0.7); 3.493(0.7); 3.440(0.7); 3.397(0.7); 3.218(0.3); 3.186(0.9); 2.676(0.5); 2.671(0.7); 2.666(0.5); 2.524(1.3); 2.519(2.1); 2.511(36.6); 2.506(76.2); 2.502(101.9); 2.497(75.3); 2.493(37.2); 2.333(0.5); 2.329(0.7); 2.324(0.5); 2.073(2.0); 0.008(0.9); 0.000(33.5); −0.008(1.2) |
| 120 | 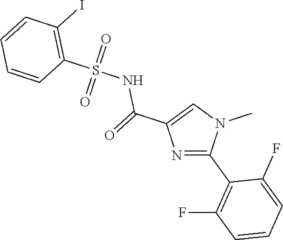 [SMILES: Cn1cc(C(=O)NS(=O)(=O)c2ccccc2I)nc1-c1c(F)cccc1F] | Example 120: ¹H-NMR(400.0 MHz, d₆-DMSO): d = 8.265(7.2); 8.189(2.3); 8.185(2.4); 8.169(2.6); 8.166(2.6); 8.137(2.5); 8.134(2.6); 8.117(2.8); 8.115 (2.7); 7.745(0.5); 7.728(1.0); 7.724(0.9); 7.711(0.8); 7.707(1.8); 7.690(1.0); 7.686(1.1); 7.673(1.5); 7.670(1.8); 7.653(2.3); 7.652(2.4); 7.634(1.5); 7.632 (1.4); 7.383(1.4); 7.379(1.4); 7.364(2.7); 7.360(2.8); 7.355(3.5); 7.345(1.7); 7.341(2.1); 7.335(4.7); 7.314(2.7); 7.307(0.6); 3.581(16.0); 2.525(0.3); 2.520(0.6); 2.511(12.2); 2.507(25.2); 2.502(33.5); 2.498(24.7); 2.493(12.3); 2.074(2.4); 0.008(0.4); 0.000(16.4); −0.009(0.7) |
| 121 | 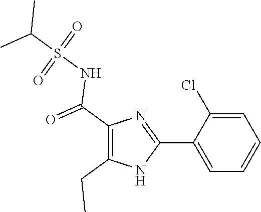 [SMILES: CCc1[nH]c(-c2ccccc2Cl)nc1C(=O)NS(=O)(=O)C(C)C] | Example 121: ¹H-NMR(400.0 MHz, d₆-DMSO): d = 12.892(0.7); 7.807(1.7); 7.802(1.3); 7.800(1.3); 7.789(2.1); 7.783(2.0); 7.619(1.4); 7.614(1.7); 7.601 (1.3); 7.599(1.6); 7.596(2.2); 7.522(0.6); 7.517(0.8); 7.504(1.9); 7.498(1.8); 7.488(2.1); 7.486(2.2); 7.483(2.1); 7.479(1.6); 7.470(1.9); 7.466(1.6); 7.451 (0.6); 7.447(0.5); 3.811(0.4); 3.794(1.1); 3.777(1.5); 3.760(1.1); 3.743(0.5); 2.987(0.7); 2.968(2.1); 2.950(2.2); 2.931(0.8); 2.524(0.4); 2.520(0.6); 2.511 (12.4); 2.506(26.8); 2.502(36.3); 2.497(26.0); 2.493(12.3); 1.322(16.0); 1.304(15.8); 1.235(3.7); 1.216(8.3); 1.198(3.6); 0.008(1.7); 0.000(55.5); −0.009(2.0) |
| 122 | 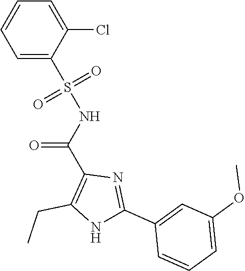 [SMILES: CCc1[nH]c(-c2cccc(OC)c2)nc1C(=O)NS(=O)(=O)c1ccccc1Cl] | Example 122: ¹H-NMR(400.0 MHz, d₆-DMSO): d = 8.144(1.3); 8.141(1.3); 8.122(1.5); 7.653(1.7); 7.640(1.5); 7.612(2.9); 7.592(0.6); 7.577(0.9); 7.572 (0.7); 7.557(1.0); 7.542(0.5); 7.536(0.4); 7.451(1.3); 7.431(2.1); 7.412(1.1); 7.054(1.1); 7.049(1.1); 7.034(1.0); 7.028(1.0); 3.841(16.0); 2.936(0.5); 2.918(1.3); 2.899(1.4); 2.881(0.5); 2.524(0.6); 2.507(31.2); 2.502(41.1); 2.498(30.2); 1.184(2.4); 1.165(5.2); 1.147(2.4); 0.008(2.0); 0.000(50.3); −0.008(2.2) |
| 123 | 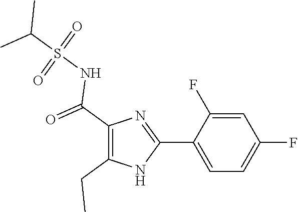 [SMILES: CCc1[nH]c(-c2ccc(F)cc2F)nc1C(=O)NS(=O)(=O)C(C)C] | Example 123: ¹H-NMR(400.0 MHz, d₆-DMSO): d = 12.747(0.7); 8.134(2.4); 8.120(1.3); 7.477(1.3); 7.471(1.4); 7.449(2.5); 7.426(1.3); 7.420(1.3); 7.270 (1.2); 7.265(1.3); 7.249(2.3); 7.244(2.3); 7.228(1.2); 3.786(1.2); 3.770(1.5); 3.754(1.3); 3.662(0.5); 3.363(3.6); 3.149(0.8); 3.123(0.7); 3.054(0.4); 3.019 (0.4); 2.976(1.7); 2.958(4.5); 2.939(4.6); 2.921(1.8); 2.676(0.5); 2.672(0.6); 2.667(0.5); 2.525(1.3); 2.511(32.7); 2.507(67.5); 2.503(91.7); 2.498(70.9); 2.494(37.5); 2.334(0.5); 2.329(0.6); 2.325(0.5); 1.315(14.9); 1.298(15.0); 1.214(7.5); 1.195(16.0); 1.177(7.4); 1.151(0.4); 1.133(0.4); 0.008(0.7); 0.000(21.3) |
| 124 | 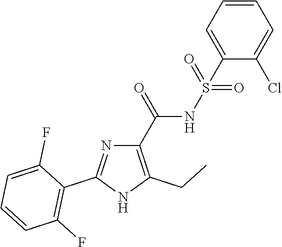 [SMILES: CCc1[nH]c(-c2c(F)cccc2F)nc1C(=O)NS(=O)(=O)c1ccccc1Cl] | Example 124: ¹H-NMR(400.0 MHz, d₆-DMSO): d = 13.287(0.7); 8.157(3.2); 8.154(3.3); 8.136(3.7); 8.134(3.6); 7.705(0.7); 7.701(0.7); 7.685(2.5); 7.681 (2.3); 7.663(6.8); 7.658(6.7); 7.640(3.5); 7.618(3.7); 7.601(2.9); 7.598(3.1); 7.582(1.4); 7.577(1.2); 7.334(4.5); 7.314(7.8); 7.293(3.8); 4.420(0.3); 4.415 (0.3); 4.403(0.4); 4.255(0.9); 4.151(1.3); 4.146(1.3); 3.186(0.8); 2.891(1.7); 2.872(5.2); 2.853(5.4); 2.834(1.9); 2.672(0.3); 2.525(0.5); 2.507(48.3); 2.503(61.9); 2.498(45.4); 2.330(0.4); 2.326(0.3); 1.148(7.4); 1.129(16.0); 1.110(7.3); 0.000(2.4) |
| 125 | 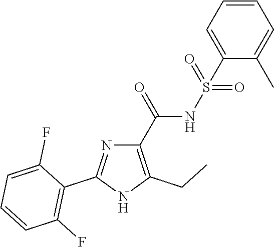 [SMILES: CCc1[nH]c(-c2c(F)cccc2F)nc1C(=O)NS(=O)(=O)c1ccccc1C] | Example 125: ¹H-NMR(400.0 MHz, d₆-DMSO): d = 13.096(0.5); 8.036(1.9); 8.019(2.0); 8.016(2.0); 7.654(0.3); 7.637(0.8); 7.633(0.8); 7.616(1.4); 7.600 (0.8); 7.595(0.9); 7.579(1.0); 7.561(1.5); 7.543(1.0); 7.453(1.1); 7.434(1.7); 7.415(0.8); 7.398(1.8); 7.380(1.5); 7.314(2.3); 7.293(4.0); 7.273(2.0); 3.727 (0.5); 3.482(16.9); 3.265(0.4); 3.247(0.3); 3.185(0.6); 2.866(1.2); 2.847(3.7); 2.828(3.8); 2.809(1.3); 2.613(16.0); 2.527(0.7); 2.513(17.0); 2.509(35.6); 2.504(47.8); 2.500(34.9); 2.495(17.2); 2.073(0.8); 1.138(5.2); 1.119(11.6); 1.100(5.1); 0.000(4.2) |
| 126 | 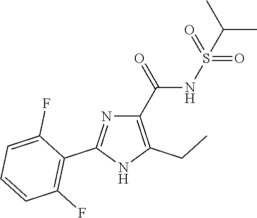 [SMILES: CCc1[nH]c(-c2c(F)cccc2F)nc1C(=O)NS(=O)(=O)C(C)C] | Example 126: ¹H-NMR(400.0 MHz, d₆-DMSO): d = 13.090(0.9); 7.646(0.3); 7.625(1.0); 7.608(1.6); 7.588(1.1); 7.571(0.4); 7.312(2.5); 7.291(4.3); 7.271 (2.1); 5.755(5.9); 3.800(0.5); 3.784(1.2); 3.767(1.5); 3.750(1.2); 3.733(0.6); 3.458(0.3); 3.444(0.4); 3.427(0.4); 3.424(0.4); 3.418(0.4); 3.406(0.4); 3.397 (0.4); 3.393(0.4); 3.377(0.4); 3.364(0.4); 3.349(0.4); 3.326(0.4); 3.174(1.3); 2.989(1.2); 2.970(3.4); 2.951(3.5); 2.933(1.4); 2.506(32.5); 2.502(40.2); 2.498(32.3); 2.086(1.8); 1.312(16.0); 1.295(15.9); 1.271(0.9); 1.253(0.5); 1.232(4.6); 1.213(9.0); 1.194(4.4); 1.142(0.4); 0.000(18.4) |
| 127 | 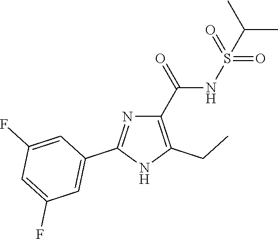 [SMILES: CCc1[nH]c(-c2cc(F)cc(F)c2)nc1C(=O)NS(=O)(=O)C(C)C] | Example 127: ¹H-NMR(400.0 MHz, d₆-DMSO): d = 13.115(1.1); 7.811(0.4); 7.793(2.3); 7.776(2.3); 7.329(0.5); 7.324(0.7); 7.318(0.5); 7.306(0.9); 7.301 (1.5); 7.295(0.8); 7.283(0.5); 7.278(0.7); 3.824(0.4); 3.807(1.1); 3.790(1.5); 3.773(1.2); 3.756(0.5); 3.338(8.6); 2.991(0.9); 2.972(2.8); 2.953(2.9); 2.934 (1.0); 2.678(0.4); 2.532(0.8); 2.518(23.2); 2.514(49.0); 2.509(66.2); 2.505 (48.3); 2.500(23.5); 2.336(0.4); 2.081(2.4); 1.335(16.0); 1.318(15.8); 1.256 (3.6); 1.237(7.8); 1.218(3.6) |
| 128 | 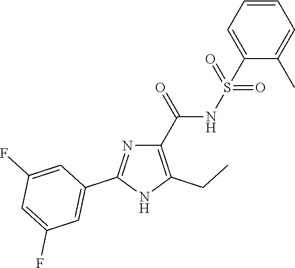 [SMILES: CCc1[nH]c(-c2cc(F)cc(F)c2)nc1C(=O)NS(=O)(=O)c1ccccc1C] | Example 128: ¹H-NMR(400.0 MHz, d₆-DMSO): d = 13.069(0.9); 8.041(1.7); 8.021(1.8); 7.808(2.0); 7.791(2.0); 7.589(0.5); 7.571(1.2); 7.552(0.8); 7.464 (0.8); 7.444(1.3); 7.426(0.7); 7.405(1.4); 7.387(1.2); 7.321(0.6); 7.298(1.2); 7.275(0.6); 3.571(0.3); 3.379(0.9); 3.357(0.9); 3.341(0.9); 3.209(0.5); 3.185 (0.7); 3.153(0.3); 2.851(1.1); 2.832(3.1); 2.814(3.2); 2.795(1.1); 2.676(0.4); 2.671(0.5); 2.667(0.4); 2.634(13.8); 2.524(1.1); 2.511(25.6); 2.507(53.3); 2.502(71.4); 2.498(51.7); 2.493(24.9); 2.329(0.4); 2.074(16.0); 1.150(4.6); 1.131(10.3); 1.112(4.5); 0.000(0.9) |
| 129 | 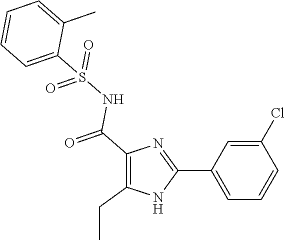 [SMILES: CCc1[nH]c(-c2cccc(Cl)c2)nc1C(=O)NS(=O)(=O)c1ccccc1C] | Example 129: ¹H-NMR(400.0 MHz, d₆-DMSO): d = 13.046(0.7); 8.165(2.3); 8.044(2.1); 8.041(2.1); 8.024(2.3); 8.021(2.3); 8.002(1.9); 7.982(2.1); 7.583 (0.7); 7.565(1.7); 7.549(2.4); 7.529(3.3); 7.510(2.5); 7.484(2.3); 7.479(1.9); 7.462(2.1); 7.441(1.8); 7.421(0.9); 7.402(2.0); 7.384(1.7); 3.775(0.3); 3.748 (0.4); 3.579(0.5); 3.558(0.5); 3.547(0.5); 3.511(0.5); 3.498(0.5); 3.439(0.5); 3.340(0.4); 3.308(0.3); 3.185(0.9); 2.853(1.0); 2.835(3.0); 2.816(3.1); 2.797 (1.2); 2.672(0.4); 2.638(16.0); 2.525(0.7); 2.520(1.2); 2.512(18.3); 2.507 (39.2); 2.503(55.0); 2.498(40.9); 2.494(19.4); 2.330(0.4); 2.075(0.7); 1.150 (4.9); 1.132(11.2); 1.113(4.8); 0.008(1.2); 0.000(38.5); −0.009(1.2) |
| 130 | 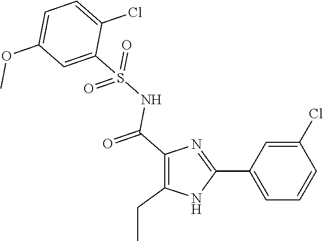 [SMILES: CCc1[nH]c(-c2cccc(Cl)c2)nc1C(=O)NS(=O)(=O)c1cc(OC)ccc1Cl] | Example 130: ¹H-NMR(400.0 MHz, d₆-DMSO): d = 8.169(1.7); 8.005(1.4); 7.990(1.0); 7.987(1.5); 7.615(2.9); 7.608(3.1); 7.560(2.8); 7.539(3.2); 7.522 (1.9); 7.512(1.3); 7.509(1.9); 7.505(1.4); 7.493(0.6); 7.488(0.7); 7.266(1.2); 7.258(1.2); 7.244(1.1); 7.236(1.1); 4.070(0.4); 4.032(0.6); 4.004(0.6); 3.989 (0.6); 3.970(0.6); 3.960(0.6); 3.884(0.5); 3.853(16.0); 3.811(0.4); 2.895(0.6); 2.877(1.9); 2.858(1.9); 2.840(0.7); 2.525(0.5); 2.512(12.6); 2.507(26.6); 2.503(37.4); 2.498(28.8); 2.494(14.8); 2.075(1.0); 1.172(2.9); 1.153(6.5); 1.134(2.9); 0.008(0.3); 0.000(9.4); −0.008(0.5) |
| 131 | 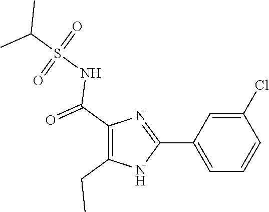 [SMILES: CCc1[nH]c(-c2cccc(Cl)c2)nc1C(=O)NS(=O)(=O)C(C)C] | Example 131: ¹H-NMR(400.0 MHz, d₆-DMSO): d = 13.063(1.1); 8.148(2.5); 7.989(1.6); 7.970(1.8); 7.542(1.2); 7.522(2.8); 7.502(2.1); 7.475(1.8); 7.453 (0.9); 3.823(0.4); 3.806(1.1); 3.789(1.5); 3.772(1.1); 3.754(0.4); 3.345(0.5); 2.983(0.8); 2.964(2.4); 2.945(2.5); 2.927(0.9); 2.525(0.6); 2.512(12.7); 2.507 (26.9); 2.503(37.9); 2.498(28.3); 2.494(13.6); 2.074(0.7); 1.331(16.0); 1.314 (16.0); 1.250(3.4); 1.231(7.3); 1.212(3.3); 0.008(0.6); 0.000(18.1); −0.008 (0.6) |
| 132 | 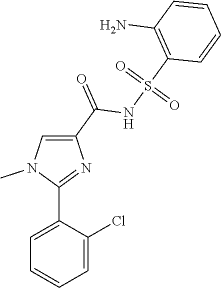 [SMILES: Cn1cc(C(=O)NS(=O)(=O)c2ccccc2N)nc1-c1ccccc1Cl] | Example 132: ¹H-NMR(600.1 MHz, d₆-DMSO): d = 8.135(1.6); 8.040(16.0); 7.684(0.5); 7.669(6.8); 7.667(7.7); 7.664(7.0); 7.662(6.7); 7.656(7.9); 7.653 (8.6); 7.650(9.8); 7.649(8.9); 7.639(1.1); 7.625(1.1); 7.623(1.1); 7.617(1.2); 7.610(4.0); 7.607(4.5); 7.598(5.4); 7.596(5.1); 7.595(6.3); 7.584(3.8); 7.580 (6.6); 7.577(5.5); 7.567(8.5); 7.564(6.6); 7.552(0.9); 7.543(0.6); 7.538(0.6); 7.521(5.1); 7.519(5.1); 7.508(6.7); 7.507(6.4); 7.496(2.9); 7.494(2.7); 7.289 (3.1); 7.287(3.2); 7.277(4.3); 7.275(5.9); 7.273(4.3); 7.264(3.5); 7.261(3.4); 6.749(6.9); 6.748(7.1); 6.735(6.8); 6.734(6.7); 6.617(3.9); 6.616(3.9); 6.604 (7.1); 6.592(3.8); 6.590(3.6); 3.613(0.5); 3.576(0.8); 3.553(0.5); 3.497(53.1); 3.403(0.5); 3.377(0.8); 3.353(0.6); 3.171(0.6); 2.616(0.4); 2.613(0.5); 2.610 (0.4); 2.522(1.6); 2.519(2.1); 2.507(27.2); 2.504(51.5); 2.501(67.5); 2.498 (51.7); 2.495(27.9); 2.388(0.4); 2.385(0.5); 2.382(0.4); 0.000(5.1) |
| 133 | 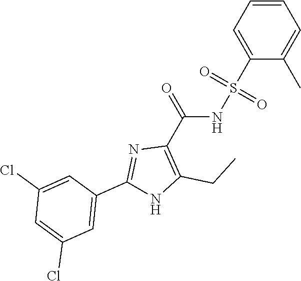 [SMILES: CCc1[nH]c(-c2cc(Cl)cc(Cl)c2)nc1C(=O)NS(=O)(=O)c1ccccc1C] | Example 133: ¹H-NMR(400.0 MHz, d₆-DMSO): d = 8.139(1.6); 8.135(1.6); 8.039(0.5); 8.019(0.5); 7.639(0.7); 7.401(0.3); 2.831(0.9); 2.812(0.9); 2.794 (0.3); 2.636(3.6); 2.512(5.9); 2.507(11.9); 2.503(16.3); 2.498(12.2); 2.494 (6.0); 2.086(16.0); 1.149(1.3); 1.130(2.8); 1.111(1.2); 0.000(3.5) |
| 134 | 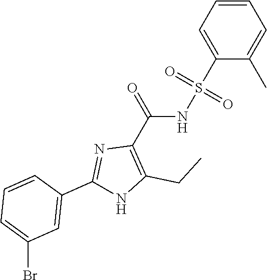 [SMILES: CCc1[nH]c(-c2cccc(Br)c2)nc1C(=O)NS(=O)(=O)c1ccccc1C] | Example 134: ¹H-NMR(400.0 MHz, d₆-DMSO): d = 13.039(1.5); 8.297(3.2); 8.044(4.4); 8.025(4.8); 7.618(1.8); 7.598(2.4); 7.587(1.3); 7.567(2.4); 7.549 (1.6); 7.480(2.0); 7.460(4.6); 7.441(3.7); 7.423(1.2); 7.404(2.7); 7.385(2.2); 2.852(1.1); 2.834(3.1); 2.815(3.2); 2.796(1.3); 2.671(0.4); 2.639(16.0); 2.503 (46.9); 2.074(8.1); 2.073(7.5); 1.149(4.2); 1.131(8.7); 1.112(4.2); 0.000(6.6) |
| 135 | 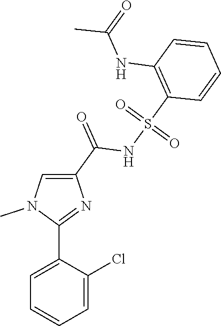 [SMILES: CC(=O)Nc1ccccc1S(=O)(=O)NC(=O)c1cn(C)c(-c2ccccc2Cl)n1] | Example 135: ¹H-NMR(601.6 MHz, d₆-DMSO): d = 9.718(1.0); 8.239(1.4); 8.225(1.5); 8.137(4.8); 7.981(1.7); 7.979(1.7); 7.968(1.8); 7.965(1.8); 7.689 (1.4); 7.675(2.7); 7.643(1.1); 7.640(1.3); 7.630(1.5); 7.627(3.4); 7.615(4.1); 7.600(1.6); 7.588(0.9); 7.586(0.8); 7.544(1.4); 7.542(1.4); 7.532(2.1); 7.519 (0.9); 7.517(0.9); 7.281(1.0); 7.280(1.0); 7.268(1.9); 7.256(1.0); 7.254(0.9); 3.525(15.8); 2.508(5.5); 2.505(11.5); 2.502(15.5); 2.499(11.8); 2.497(5.8); 2.144(16.0); 0.000(1.3) |
| 136 | 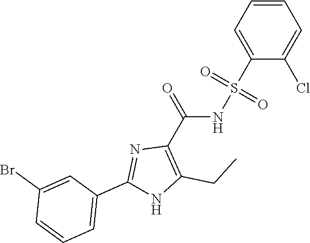 [SMILES: CCc1[nH]c(-c2cccc(Br)c2)nc1C(=O)NS(=O)(=O)c1ccccc1Cl] | Example 136: ¹H-NMR(400.0 MHz, d₆-DMSO): d = 13.189(0.5); 8.309(1.6); 8.167(1.4); 8.148(1.5); 8.056(1.2); 8.037(1.3); 7.690(1.0); 7.668(2.2); 7.663 (2.2); 7.647(1.7); 7.627(2.2); 7.608(1.2); 7.591(0.5); 7.587(0.5); 7.496(1.3); 7.477(2.1); 7.457(1.0); 3.656(0.7); 3.646(0.7); 2.882(0.6); 2.863(1.7); 2.844 (1.8); 2.825(0.7); 2.679(0.7); 2.513(79.1); 2.510(104.0); 2.506(81.9); 2.337 (0.7); 2.082(16.0); 1.220(0.5); 1.165(2.7); 1.146(5.6); 1.127(2.6) |
| 137 | 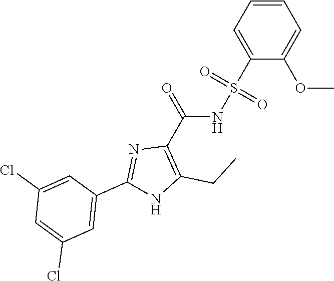 [SMILES: CCc1[nH]c(-c2cc(Cl)cc(Cl)c2)nc1C(=O)NS(=O)(=O)c1ccccc1OC] | Example 137: ¹H-NMR(400.0 MHz, d₆-DMSO): d = 13.099(1.7); 8.134(7.2); 8.039(1.1); 7.906(1.9); 7.887(1.9); 7.684(0.9); 7.651(3.4); 7.615(0.5); 7.473 (0.5); 7.353(1.0); 7.239(1.9); 7.219(1.7); 7.169(1.1); 7.151(1.9); 7.131(1.0); 4.419(0.5); 4.403(0.5); 3.899(0.4); 3.850(11.4); 3.317(15.0); 2.995(0.5); 2.977(0.5); 2.855(1.1); 2.836(2.9); 2.817(3.0); 2.799(1.2); 2.670(1.3); 2.501 (190.6); 2.329(1.1); 2.073(16.0); 1.230(0.5); 1.211(1.0); 1.192(0.5); 1.149 (3.4); 1.130(7.0); 1.111(3.5); 0.146(0.3); 0.000(61.8); −0.150(0.3) |
| 138 | 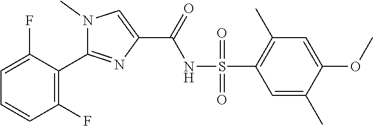 [SMILES: COc1cc(C)c(S(=O)(=O)NC(=O)c2cn(C)c(-c3c(F)cccc3F)n2)cc1C] | Example 138: ¹H-NMR(400.0 MHz, d₆-DMSO): d = 8.151(2.5); 7.772(4.2); 7.736(0.4); 7.719(0.8); 7.715(0.8); 7.698(1.4); 7.682(0.8); 7.677(0.9); 7.661 (0.4); 7.347(2.5); 7.327(4.0); 7.307(2.2); 6.925(3.7); 3.854(16.0); 3.557 (13.4); 3.319(7.4); 2.670(0.9); 2.568(12.6); 2.505(108.1); 2.501(143.5); 2.497(108.6); 2.328(0.9); 2.165(12.2); 2.073(1.4); 1.236(0.3); 0.146(0.7); 0.007(6.6); 0.000(153.5); −0.150(0.8) |
| 139 | 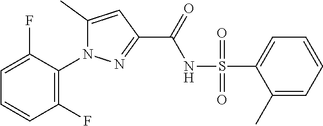 [SMILES: Cc1ccccc1S(=O)(=O)NC(=O)c1cc(C)n(-c2c(F)cccc2F)n1] | Example 139: ¹H-NMR(400.0 MHz, d₆-DMSO): d = 12.616(0.6); 8.040(2.2); 8.021(2.3); 7.785(0.4); 7.769(0.9); 7.763(0.8); 7.747(1.6); 7.731(0.9); 7.726 (1.0); 7.710(0.5); 7.601(0.9); 7.582(2.1); 7.565(1.4); 7.476(2.7); 7.464(1.8); 7.456(4.8); 7.447(2.6); 7.435(2.4); 7.411(2.3); 7.392(1.9); 6.843(4.6); 3.322 (26.8); 2.671(0.4); 2.604(16.0); 2.506(48.7); 2.502(66.3); 2.498(50.6); 2.329 (0.4); 2.155(14.9); 2.073(1.4); 0.008(2.1); 0.000(56.8); −0.008(2.5) |
| 141 | 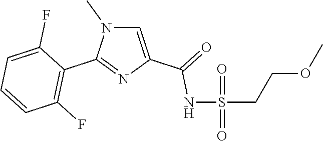 [SMILES: COCCS(=O)(=O)NC(=O)c1cn(C)c(-c2c(F)cccc2F)n1] | Example 141: ¹H-NMR(400.0 MHz, CDCl₃): d = 7.835(4.3); 7.544(0.6); 7.538(0.6); 7.528(0.4); 7.522(1.2); 7.517(0.4); 7.506(0.6); 7.501(0.7); 7.266 (8.2); 7.110(0.3); 7.104(1.9); 7.085(2.3); 7.083(2.3); 7.064(1.7); 5.302(0.5); 3.881(1.2); 3.866(3.3); 3.852(2.3); 3.777(2.3); 3.763(3.1); 3.749(1.1); 3.747 (1.1); 3.639(9.1); 3.325(16.0) |
| 142 | 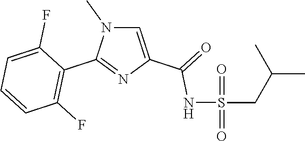 [SMILES: CC(C)CS(=O)(=O)NC(=O)c1cn(C)c(-c2c(F)cccc2F)n1] | Example 142: ¹H-NMR(400.0 MHz, d₆-DMSO): d = 8.253(4.8); 7.729(0.7); 7.725(0.8); 7.708(1.4); 7.691(0.8); 7.688(0.8); 7.671(0.3); 7.359(2.3); 7.339 (3.6); 7.319(1.9); 3.589(12.2); 3.397(4.9); 3.381(5.2); 3.320(3.9); 2.670(0.5); 2.505(55.3); 2.501(74.3); 2.497(59.5); 2.328(0.5); 2.188(0.5); 2.171(0.9); 2.154(1.2); 2.138(1.0); 2.121(0.5); 1.042(16.0); 1.025(15.6); 0.000(12.6) |
| 143 | 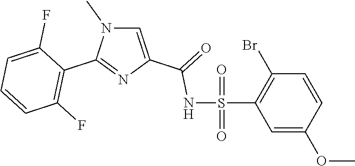 [SMILES: COc1ccc(Br)c(S(=O)(=O)NC(=O)c2cn(C)c(-c3c(F)cccc3F)n2)c1] | Example 143: ¹H-NMR(400.0 MHz, d₆-DMSO): d = 8.252(4.0); 7.746(0.5); 7.734(2.8); 7.712(3.5); 7.691(0.8); 7.688(0.9); 7.670(0.4); 7.652(3.0); 7.644 (3.2); 7.357(2.3); 7.336(3.6); 7.316(1.9); 7.210(1.5); 7.202(1.5); 7.188(1.4); 7.180(1.4); 3.850(16.0); 3.580(12.0); 2.671(0.5); 2.506(51.5); 2.501(69.5); 2.497(57.4); 2.328(0.4); 0.000(11.5) |
| 144 | 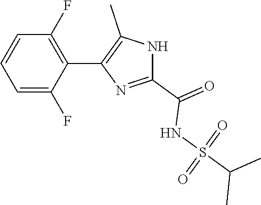 [SMILES: Cc1[nH]c(C(=O)NS(=O)(=O)C(C)C)nc1-c1c(F)cccc1F] | Example 144: ¹H-NMR(400.0 MHz, d₆-DMSO): d = 7.599(0.3); 7.583(0.7); 7.578(0.7); 7.566(0.5); 7.562(1.4); 7.545(0.7); 7.541(0.9); 7.524(0.4); 7.264 (2.3); 7.256(0.4); 7.253(0.4); 7.244(3.4); 7.224(2.0); 5.753(6.7); 4.039(0.6); 4.021(0.6); 3.736(0.4); 3.719(1.2); 3.702(1.7); 3.685(1.2); 3.668(0.5); 2.521 (0.5); 2.512(11.1); 2.507(24.7); 2.503(34.0); 2.498(24.4); 2.494(11.4); 2.169 (11.5); 1.989(2.5); 1.909(8.5); 1.293(16.0); 1.276(15.8); 1.259(0.5); 1.235 (0.4); 1.193(0.7); 1.176(1.4); 1.158(0.7); 0.008(2.2); 0.000(74.3); −0.009 (2.4) |
| 145 | 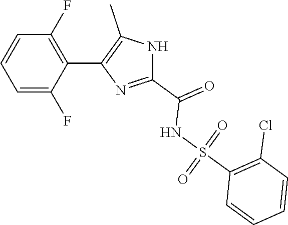 [SMILES: Cc1[nH]c(C(=O)NS(=O)(=O)c2ccccc2Cl)nc1-c1c(F)cccc1F] | Example 145: ¹H-NMR(400.0 MHz, d₆-DMSO): d = 8.133(0.5); 8.096(2.2); 8.094(2.1); 8.076(2.9); 7.676(0.5); 7.659(1.0); 7.655(0.9); 7.638(1.8); 7.622 (1.0); 7.617(1.1); 7.601(0.5); 7.505(1.1); 7.501(1.9); 7.495(2.8); 7.489(7.4); 7.478(3.3); 7.471(1.3); 7.459(1.9); 7.451(1.1); 7.446(1.0); 7.437(0.7); 7.321 (3.2); 7.300(5.2); 7.280(2.6); 4.340(0.3); 4.253(0.4); 4.216(0.4); 4.204(0.5); 4.193(0.4); 4.166(0.5); 4.129(0.5); 4.056(0.7); 4.038(0.9); 4.020(0.9); 4.003 (0.8); 3.815(1.1); 3.759(1.2); 3.732(1.2); 3.617(0.8); 3.490(0.3); 2.675(0.4); 2.671(0.5); 2.667(0.4); 2.524(0.8); 2.511(34.0); 2.507(71.4); 2.502(95.5); 2.498(69.7); 2.338(0.3); 2.333(0.5); 2.329(0.6); 2.324(0.5); 2.179(16.0); 1.988(1.3); 1.193(0.3); 1.175(0.7); 1.157(0.3); 0.000(8.1) |
| 146 | 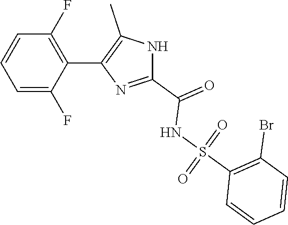 [SMILES: Cc1[nH]c(C(=O)NS(=O)(=O)c2ccccc2Br)nc1-c1c(F)cccc1F] | Example 146: ¹H-NMR(400.0 MHz, d₆-DMSO): d = 8.313(0.3); 8.123(2.6); 8.119(2.8); 8.103(2.9); 8.099(2.9); 7.686(2.5); 7.683(2.9); 7.675(0.6); 7.666 (3.1); 7.664(3.3); 7.658(1.1); 7.654(1.0); 7.637(1.7); 7.620(0.9); 7.616(1.1); 7.599(0.5); 7.524(1.2); 7.521(1.4); 7.505(2.6); 7.503(2.7); 7.486(1.7); 7.483 (1.8); 7.414(1.6); 7.410(1.8); 7.395(2.3); 7.390(2.4); 7.376(1.1); 7.372(1.1); 7.320(3.1); 7.300(5.0); 7.279(2.6); 4.115(0.3); 4.084(0.3); 4.056(0.4); 4.038 (0.5); 4.020(0.5); 4.002(0.4); 3.992(0.3); 3.971(0.3); 3.944(0.3); 3.938(0.3); 3.921(0.3); 3.910(0.3); 3.903(0.3); 3.879(0.3); 2.675(0.7); 2.670(1.0); 2.666 (0.7); 2.524(2.0); 2.519(3.3); 2.510(59.0); 2.506(128.1); 2.501(175.5); 2.497(128.3); 2.492(62.6); 2.430(0.4); 2.337(0.5); 2.333(0.8); 2.328(1.1); 2.323(0.9); 2.179(16.0); 1.988(0.8); 1.175(0.4); 0.008(0.5); 0.000(17.1); −0.009(0.7) |
| 147 | 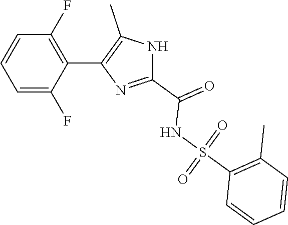 [SMILES: Cc1ccccc1S(=O)(=O)NC(=O)c1nc(-c2c(F)cccc2F)c(C)[nH]1] | Example 147: ¹H-NMR(400.0 MHz, d₆-DMSO): d = 7.986(2.2); 7.969(2.3); 7.967(2.2); 7.644(0.4); 7.627(1.0); 7.623(0.9); 7.606(1.6); 7.590(0.9); 7.585 (1.0); 7.569(0.4); 7.448(0.9); 7.432(2.2); 7.429(2.1); 7.413(1.5); 7.411(1.4); 7.345(1.4); 7.327(2.2); 7.307(1.3); 7.294(5.0); 7.275(6.4); 7.254(2.3); 7.246 (0.4); 5.753(8.3); 4.057(0.3); 4.039(1.0); 4.022(1.0); 4.004(0.4); 2.601(16.0); 2.526(0.3); 2.508(22.0); 2.504(29.1); 2.499(21.5); 2.158(14.2); 1.989(4.6); 1.910(12.0); 1.235(0.5); 1.194(1.2); 1.176(2.4); 1.158(1.2); 0.008(1.6); 0.000 (53.2); −0.008(2.8) |
| 148 | 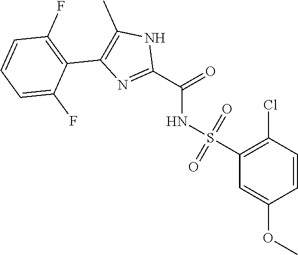 [SMILES: COc1ccc(Cl)c(S(=O)(=O)NC(=O)c2nc(-c3c(F)cccc3F)c(C)[nH]2)c1] | Example 148: ¹H-NMR(400.0 MHz, d₆-DMSO): d = 7.661(0.6); 7.658(0.5); 7.641(1.0); 7.623(0.6); 7.620(0.6); 7.607(2.8); 7.600(2.9); 7.395(2.4); 7.373 (2.7); 7.323(1.8); 7.302(3.0); 7.282(1.5); 7.087(1.4); 7.080(1.3); 7.066(1.2); 7.058(1.2); 5.754(8.2); 4.056(0.6); 4.038(1.0); 4.021(1.1); 4.003(0.6); 3.990 (0.5); 3.963(0.5); 3.946(0.5); 3.907(0.5); 3.811(16.0); 3.730(0.7); 3.628 (0.5); 2.524(0.6); 2.506(42.9); 2.502(55.7); 2.498(39.8); 2.329(0.4); 2.180 (9.0); 1.988(3.0); 1.909(5.2); 1.236(0.3); 1.193(0.8); 1.175(1.6); 1.157(0.8); 0.008(2.5); 0.000(71.6); −0.008(2.6); −0.150(0.4) |
| 149 | 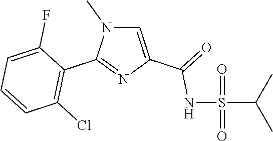 [SMILES: CC(C)S(=O)(=O)NC(=O)c1cn(C)c(-c2c(F)cccc2Cl)n1] | Example 149: ¹H-NMR(400.0 MHz, d₆-DMSO): d = 8.257(4.8); 7.713(0.7); 7.698(0.8); 7.692(1.6); 7.677(1.7); 7.672(1.1); 7.656(1.0); 7.574(2.6); 7.554 (1.8); 7.489(1.1); 7.487(1.1); 7.466(2.0); 7.445(0.9); 7.443(0.9); 3.802(0.4); 3.785(1.2); 3.768(1.6); 3.751(1.2); 3.734(0.5); 3.539(16.0); 3.322(12.3); 2.675(0.4); 2.670(0.5); 2.666(0.4); 2.524(1.4); 2.519(2.3); 2.510(31.7); 2.506 (66.8); 2.501(89.9); 2.497(64.4); 2.492(30.7); 2.333(0.4); 2.328(0.5); 2.324 (0.4); 1.312(14.5); 1.295(14.3); 0.000(1.2) |
| 150 | 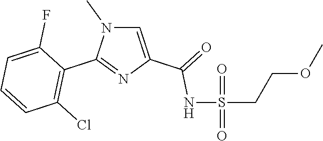 [SMILES: COCCS(=O)(=O)NC(=O)c1cn(C)c(-c2c(F)cccc2Cl)n1] | Example 150: ¹H-NMR(400.0 MHz, d₆-DMSO): d = 8.251(4.6); 7.713(0.5); 7.693(1.2); 7.677(1.3); 7.672(1.0); 7.657(0.8); 7.573(2.3); 7.553(1.7); 7.487 (1.1); 7.465(1.9); 7.444(0.9); 3.725(16.0); 3.540(12.2); 3.382(0.6); 3.358 (0.7); 3.328(0.7); 3.208(15.0); 2.501(42.1); 0.000(5.9) |
| 151 | 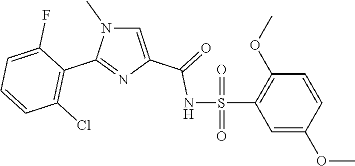 [SMILES: COc1ccc(OC)c(S(=O)(=O)NC(=O)c2cn(C)c(-c3c(F)cccc3Cl)n2)c1] | Example 151: ¹H-NMR(400.0 MHz, d₆-DMSO): d = 8.217(1.6); 7.712(0.5); 7.696(0.6); 7.691(1.1); 7.676(1.1); 7.671(0.8); 7.655(0.7); 7.572(2.0); 7.551 (1.4); 7.487(0.9); 7.465(1.5); 7.444(0.7); 7.381(2.5); 7.373(2.9); 7.268(0.6); 7.261(0.5); 7.246(1.0); 7.238(0.9); 7.190(1.9); 7.167(1.0); 3.787(16.0); 3.778(13.4); 3.519(11.2); 3.319(4.5); 2.674(0.4); 2.670(0.6); 2.665(0.4); 2.523(1.3); 2.518(2.0); 2.510(34.0); 2.505(72.2); 2.501(97.4); 2.496(69.9); 2.492(33.3); 2.332(0.4); 2.328(0.6); 2.323(0.4); 0.000(1.3) |
| 152 | 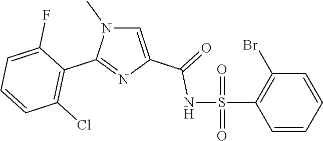 [SMILES: Cn1cc(C(=O)NS(=O)(=O)c2ccccc2Br)nc1-c1c(F)cccc1Cl] | Example 152: ¹H-NMR(400.0 MHz, d₆-DMSO): d = 8.256(5.7); 8.194(1.8); 8.190(2.1); 8.175(2.0); 8.170(2.1); 7.855(1.8); 7.852(2.0); 7.835(2.2); 7.832 (2.4); 7.711(0.7); 7.696(0.8); 7.690(1.7); 7.675(2.4); 7.670(1.6); 7.654(3.0); 7.637(1.5); 7.634(1.5); 7.615(1.4); 7.610(1.5); 7.596(1.7); 7.591(1.8); 7.577 (0.8); 7.567(2.9); 7.547(2.0); 7.482(1.3); 7.461(2.2); 7.440(1.0); 7.438(1.1); 3.529(16.0); 2.510(16.3); 2.506(34.7); 2.501(49.4); 2.497(38.9); 2.328(0.3); 2.073(1.8); 0.008(0.4); 0.000(10.8); −0.008(0.5) |
| 153 | 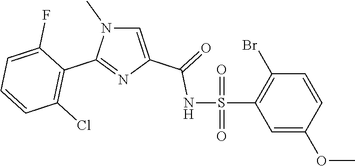 [SMILES: COc1ccc(Br)c(S(=O)(=O)NC(=O)c2cn(C)c(-c3c(F)cccc3Cl)n2)c1] | Example 153: ¹H-NMR(400.0 MHz, d₆-DMSO): d = 8.255(5.0); 7.739(2.1); 7.717(2.4); 7.698(0.6); 7.693(1.1); 7.677(1.1); 7.672(0.9); 7.656(3.6); 7.648 (3.0); 7.568(1.9); 7.548(1.4); 7.483(0.9); 7.461(1.5); 7.439(0.7); 7.214(1.2); 7.206(1.2); 7.192(1.1); 7.184(1.1); 5.751(0.8); 4.039(0.3); 4.021(0.4); 3.898 (0.3); 3.851(16.0); 3.826(0.5); 3.815(0.9); 3.800(0.6); 3.785(1.7); 3.760(0.7); 3.709(1.0); 3.666(1.5); 3.559(4.2); 3.534(15.2); 2.508(18.6); 2.504(24.9); 2.499(18.7); 1.989(0.7); 1.176(0.4); 0.000(3.1) |
| 154 | 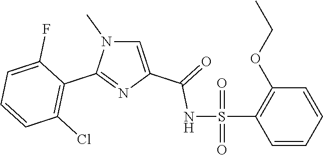 [SMILES: CCOc1ccccc1S(=O)(=O)NC(=O)c1cn(C)c(-c2c(F)cccc2Cl)n1] | Example 154: ¹H-NMR(400.0 MHz, d₆-DMSO): d = 8.198(6.7); 7.903(1.8); 7.899(1.9); 7.884(2.0); 7.879(2.0); 7.718(0.6); 7.703(0.6); 7.698(1.4); 7.682 (1.4); 7.677(1.0); 7.661(1.5); 7.643(1.4); 7.625(0.9); 7.621(0.8); 7.577(2.2); 7.557(1.6); 7.491(1.0); 7.470(1.8); 7.448(0.9); 7.218(2.1); 7.197(1.9); 7.151 (1.2); 7.132(2.0); 7.113(1.0); 5.751(0.9); 4.146(1.1); 4.129(3.8); 4.111(3.8); 4.094(1.2); 3.518(16.0); 3.354(7.7); 3.209(0.4); 2.526(0.4); 2.512(11.1); 2.508(23.4); 2.503(31.4); 2.499(22.5); 2.494(10.7); 1.270(0.3); 1.259(4.2); 1.242(8.9); 1.224(4.0); 0.000(5.8) |
| 155 | 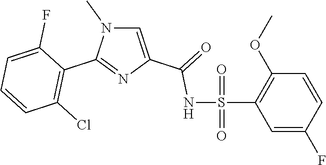 [SMILES: COc1ccc(F)cc1S(=O)(=O)NC(=O)c1cn(C)c(-c2c(F)cccc2Cl)n1] | Example 155: ¹H-NMR(400.0 MHz, d₆-DMSO): d = 8.229(3.2); 7.712(0.6); 7.696(0.7); 7.691(1.4); 7.676(1.5); 7.668(1.6); 7.659(1.7); 7.655(1.2); 7.647 (1.4); 7.639(1.5); 7.591(0.6); 7.583(0.6); 7.571(3.3); 7.563(1.0); 7.550(2.2); 7.541(0.6); 7.486(1.1); 7.464(1.9); 7.443(0.9); 7.292(1.2); 7.282(1.3); 7.269 (1.1); 7.259(1.0); 3.836(16.0); 3.651(0.3); 3.522(14.5); 3.350(3.6); 3.344 (3.6); 3.131(0.4); 2.675(0.4); 2.671(0.5); 2.666(0.4); 2.524(1.2); 2.510(26.8); 2.506(55.1); 2.502(73.7); 2.497(54.0); 2.493(27.1); 2.333(0.3); 2.329(0.4); 2.324(0.3); 2.073(1.8); 0.000(0.4) |
| 156 | 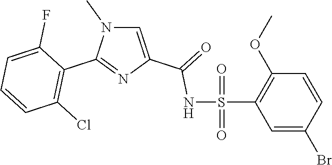 [SMILES: COc1ccc(Br)cc1S(=O)(=O)NC(=O)c1cn(C)c(-c2c(F)cccc2Cl)n1] | Example 156: ¹H-NMR(400.0 MHz, d₆-DMSO): d = 8.223(1.8); 7.935(3.5); 7.929(4.1); 7.866(1.0); 7.859(0.9); 7.843(1.1); 7.837(1.0); 7.711(0.6); 7.695 (0.7); 7.690(1.5); 7.674(1.5); 7.669(1.1); 7.654(1.0); 7.570(2.5); 7.549(1.8); 7.483(1.1); 7.463(1.9); 7.442(0.9); 7.440(0.9); 7.237(2.1); 7.215(2.0); 3.850 (16.0); 3.566(0.4); 3.521(15.0); 3.341(4.3); 2.675(0.5); 2.670(0.7); 2.666 (0.5); 2.524(1.8); 2.510(41.1); 2.506(86.4); 2.501(116.1); 2.497(83.8); 2.492 (40.1); 2.332(0.5); 2.328(0.7); 2.323(0.5); 2.073(1.1); 0.000(1.5) |
| 157 | 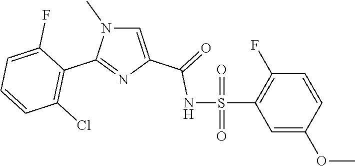 [SMILES: COc1ccc(F)c(S(=O)(=O)NC(=O)c2cn(C)c(-c3c(F)cccc3Cl)n2)c1] | Example 157: ¹H-NMR(400.0 MHz, d₆-DMSO): d = 8.228(4.8); 7.715(0.5); 7.699(0.5); 7.694(1.1); 7.678(1.1); 7.673(0.8); 7.658(0.7); 7.570(1.7); 7.549 (1.3); 7.484(0.9); 7.462(1.4); 7.440(0.7); 7.415(1.0); 7.407(1.3); 7.401(1.5); 7.393(1.2); 7.380(1.6); 7.356(1.1); 7.318(0.7); 7.308(1.2); 7.300(0.7); 7.295 (0.5); 7.286(0.7); 7.277(0.4); 5.752(0.7); 4.039(0.3); 4.021(0.4); 4.006(0.4); 3.942(0.4); 3.892(0.4); 3.869(0.4); 3.828(16.0); 3.795(0.4); 3.765(0.4); 3.530(11.5); 2.513(4.4); 2.509(9.6); 2.504(13.1); 2.499(9.6); 2.495(4.7); 1.989(0.3); 0.000(6.6) |
| 158 | 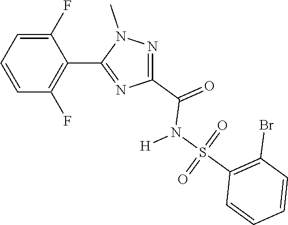 [SMILES: Cn1nc(C(=O)NS(=O)(=O)c2ccccc2Br)nc1-c1c(F)cccc1F] | Example 158: ¹H-NMR(400.0 MHz, d₆-DMSO): d = 8.213(2.1); 8.209(2.3); 8.194(2.3); 8.189(2.4); 7.874(2.1); 7.871(2.2); 7.855(2.6); 7.851(2.6); 7.828 (0.4); 7.812(0.9); 7.807(0.9); 7.790(1.7); 7.773(0.9); 7.769(1.1); 7.752(0.4); 7.692(0.9); 7.688(1.0); 7.673(2.3); 7.669(2.2); 7.654(1.8); 7.650(1.7); 7.636 (1.6); 7.631(1.8); 7.617(2.0); 7.612(2.1); 7.598(0.9); 7.593(0.8); 7.430(0.7); 7.425(3.2); 7.404(5.3); 7.384(2.6); 4.056(0.4); 4.039(1.0); 4.021(1.0); 4.003 (0.4); 3.878(16.0); 3.753(0.4); 3.737(0.4); 3.721(0.4); 3.696(0.6); 3.684(0.5); 3.579(1.2); 3.394(18.2); 3.186(1.2); 3.162(0.8); 3.062(0.4); 2.678(0.4); 2.673 (0.4); 2.669(0.3); 2.527(0.9); 2.522(1.3); 2.513(23.0); 2.509(49.7); 2.504 (67.4); 2.500(48.3); 2.495(22.8); 2.331(0.4); 1.989(4.0); 1.193(1.0); 1.175 (2.1); 1.158(1.0); 0.008(2.1); 0.000(61.7); −0.008(2.1) |
| 159 | 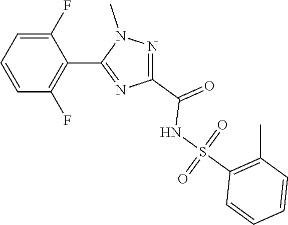 [SMILES: Cc1ccccc1S(=O)(=O)NC(=O)c1nc(-c2c(F)cccc2F)n(C)n1] | Example 159: ¹H-NMR(400.0 MHz, d₆-DMSO): d = 8.132(0.4); 8.057(1.9); 8.054(2.1); 8.037(2.1); 8.034(2.1); 7.827(0.5); 7.810(1.0); 7.805(0.9); 7.793 (0.7); 7.789(1.9); 7.784(0.7); 7.772(0.9); 7.767(1.1); 7.751(0.5); 7.618(0.8); 7.615(0.8); 7.599(1.9); 7.596(2.0); 7.580(1.3); 7.577(1.3); 7.478(1.2); 7.459 (1.9); 7.439(0.9); 7.423(5.2); 7.403(6.5); 7.382(2.7); 7.377(0.7); 4.056(0.7); 4.038(2.2); 4.020(2.2); 4.003(0.7); 3.883(1.5); 3.871(15.6); 3.330(12.3); 2.675(0.5); 2.671(0.7); 2.666(0.6); 2.627(16.0); 2.524(1.5); 2.519(2.3); 2.511 (41.1); 2.506(88.8); 2.502(121.2); 2.497(86.2); 2.492(40.2); 2.333(0.5); 2.328(0.7); 2.324(0.5); 1.988(9.8); 1.193(2.5); 1.175(5.1); 1.157(2.5); 0.146 (0.7); 0.008(5.2); 0.000(169.4); −0.009(5.2); −0.150(0.7) |
| 160 | 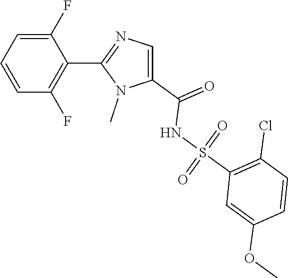 [SMILES: COc1ccc(Cl)c(S(=O)(=O)NC(=O)c2cnc(-c3c(F)cccc3F)n2C)c1] | Example 160: ¹H-NMR(400.0 MHz, d₆-DMSO): d = 8.219(2.3); 7.736(0.6); 7.731(0.5); 7.714(1.1); 7.698(0.6); 7.693(0.6); 7.623(2.9); 7.615(3.0); 7.595 (1.3); 7.573(1.5); 7.357(1.9); 7.336(3.1); 7.316(1.7); 7.296(0.8); 7.289(0.8); 7.274(0.7); 7.267(0.7); 5.756(0.5); 3.858(16.0); 3.816(0.6); 3.558(6.6); 3.352(3.1); 3.186(0.4); 2.671(0.4); 2.524(0.9); 2.511(21.9); 2.506(47.2); 2.502(67.1); 2.497(50.1); 2.493(23.9); 2.328(0.4); 0.008(0.6); 0.000(19.1); −0.009(0.6) |
| 161 | 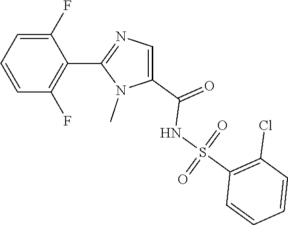 [SMILES: Cn1c(C(=O)NS(=O)(=O)c2ccccc2Cl)cnc1-c1c(F)cccc1F] | Example 161: ¹H-NMR(400.0 MHz, d₆-DMSO): d = 8.215(3.9); 8.185(3.1); 8.164(3.3); 7.748(0.6); 7.727(1.9); 7.709(4.1); 7.689(7.1); 7.672(1.9); 7.632 (1.7); 7.612(2.3); 7.596(1.2); 7.352(3.9); 7.332(6.8); 7.311(3.4); 3.665(0.9); 3.616(0.3); 3.542(16.0); 3.344(14.3); 3.186(1.0); 2.670(1.4); 2.501(224.5); 2.328(1.3); 0.000(45.7) |
| 162 | 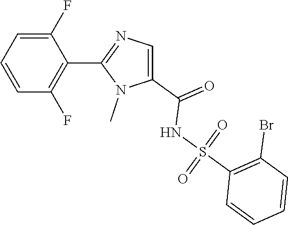 [SMILES: Cn1c(C(=O)NS(=O)(=O)c2ccccc2Br)cnc1-c1c(F)cccc1F] | Example 162: ¹H-NMR(400.0 MHz, d₆-DMSO): d = 8.233(4.9); 8.217(3.1); 8.212(3.2); 8.197(3.1); 8.193(3.2); 7.867(2.6); 7.848(3.0); 7.749(0.7); 7.732 (1.4); 7.727(1.4); 7.711(2.6); 7.694(1.5); 7.690(1.7); 7.673(1.9); 7.654(2.9); 7.637(2.1); 7.612(1.7); 7.595(2.1); 7.577(0.9); 7.353(4.8); 7.332(7.6); 7.312 (4.0); 3.544(16.0); 3.342(18.1); 2.675(0.9); 2.671(1.3); 2.666(1.0); 2.524 (3.0); 2.510(70.5); 2.506(150.1); 2.501(211.5); 2.497(158.6); 2.493(76.0); 2.333(0.9); 2.328(1.2); 2.324(0.9); 0.008(1.6); 0.000(49.6); −0.009(1.6) |
| 163 | 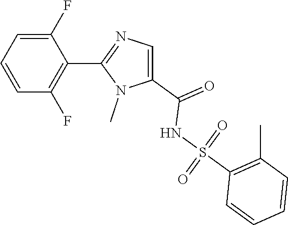 [SMILES: Cc1ccccc1S(=O)(=O)NC(=O)c1cnc(-c2c(F)cccc2F)n1C] | Example 163: ¹H-NMR(400.0 MHz, d₆-DMSO): d = 8.180(2.0); 8.051(2.2); 8.031(2.4); 7.738(0.4); 7.718(1.0); 7.700(1.7); 7.680(1.1); 7.662(0.5); 7.607 (0.8); 7.588(1.8); 7.571(1.2); 7.473(1.3); 7.453(2.0); 7.431(2.7); 7.412(1.7); 7.342(2.9); 7.322(5.0); 7.302(2.5); 3.529(14.1); 3.325(64.0); 2.670(1.4); 2.651(16.0); 2.501(206.1); 2.329(1.2); 0.000(38.5) |
| 164 | 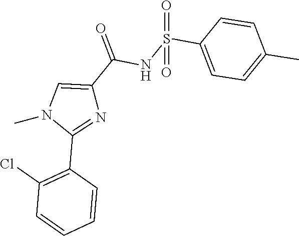 [SMILES: Cc1ccc(S(=O)(=O)NC(=O)c2cn(C)c(-c3ccccc3Cl)n2)cc1] | Example 164: ¹H-NMR(400.0 MHz, d₆-DMSO): d = 8.078(7.9); 7.885(5.5); 7.864(6.2); 7.663(1.6); 7.661(1.7); 7.643(3.2); 7.641(3.2); 7.612(1.4); 7.607 (1.7); 7.595(1.9); 7.590(2.3); 7.575(1.0); 7.570(1.5); 7.562(1.2); 7.558(1.5); 7.543(3.5); 7.539(2.4); 7.521(2.3); 7.518(2.4); 7.504(1.9); 7.501(2.0); 7.485 (0.8); 7.482(0.8); 7.428(4.7); 7.408(4.3); 3.716(0.4); 3.682(0.5); 3.667(0.6); 3.656(0.5); 3.619(0.7); 3.539(1.0); 3.534(1.1); 3.492(23.3); 3.447(1.5); 3.429(1.5); 3.385(1.5); 3.314(1.2); 3.205(0.6); 3.183(0.5); 2.675(0.4); 2.671 (0.6); 2.666(0.4); 2.524(1.7); 2.519(2.6); 2.511(30.9); 2.506(64.5); 2.501 (86.1); 2.497(61.2); 2.492(28.7); 2.393(16.0); 2.333(0.4); 2.328(0.5); 2.324 (0.4); 2.073(1.2); 0.000(1.4) |
| 165 | 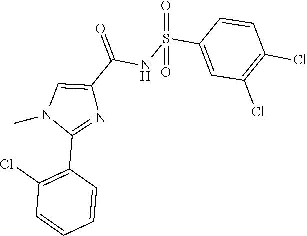 [SMILES: Cn1cc(C(=O)NS(=O)(=O)c2ccc(Cl)c(Cl)c2)nc1-c1ccccc1Cl] | Example 165: ¹H-NMR(400.0 MHz, d₆-DMSO): d = 8.137(3.0); 8.129(6.4); 7.921(0.5); 7.917(0.3); 7.900(3.5); 7.894(5.5); 7.873(0.6); 7.693(1.1); 7.690 (1.1); 7.672(2.5); 7.648(1.1); 7.643(1.3); 7.630(1.3); 7.626(1.8); 7.619(1.4); 7.615(1.3); 7.611(0.6); 7.606(1.4); 7.599(2.4); 7.596(1.4); 7.549(1.4); 7.546 (1.4); 7.530(1.6); 7.512(0.7); 7.509(0.6); 4.085(0.4); 4.072(0.4); 3.907(0.7); 3.890(0.7); 3.877(0.7); 3.701(0.4); 3.525(16.0); 2.671(0.4); 2.524(0.8); 2.519(1.3); 2.511(23.3); 2.506(49.7); 2.502(67.2); 2.497(48.3); 2.493(23.1); 2.328(0.4); 0.000(1.0) |
| 166 | 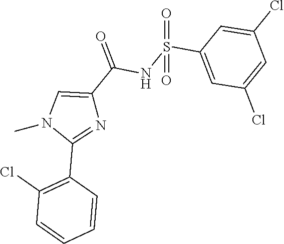 [SMILES: Cn1cc(C(=O)NS(=O)(=O)c2cc(Cl)cc(Cl)c2)nc1-c1ccccc1Cl] | Example 166: ¹H-NMR(400.0 MHz, d₆-DMSO): d = 8.149(5.4); 7.941(1.5); 7.936(3.3); 7.932(2.3); 7.898(7.9); 7.894(6.5); 7.708(1.3); 7.701(0.3); 7.689 (2.5); 7.685(2.2); 7.668(1.0); 7.664(1.4); 7.647(2.7); 7.635(2.0); 7.630(2.7); 7.564(1.3); 7.562(1.4); 7.544(1.7); 7.527(0.7); 7.525(0.7); 5.755(0.3); 4.038 (0.4); 3.801(1.8); 3.719(1.2); 3.543(16.0); 2.675(0.3); 2.671(0.4); 2.667(0.3); 2.524(0.9); 2.506(57.1); 2.502(76.8); 2.498(56.4); 2.334(0.3); 2.329(0.5); 2.325(0.4); 0.000(1.1) |
| 167 | 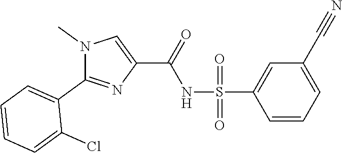 [SMILES: Cn1cc(C(=O)NS(=O)(=O)c2cccc(C#N)c2)nc1-c1ccccc1Cl] | Example 167: ¹H-NMR(400.0 MHz, d₆-DMSO): d = 8.347(1.6); 8.343(3.0); 8.339(1.9); 8.267(1.1); 8.264(1.4); 8.260(1.1); 8.247(1.2); 8.244(1.5); 8.240 (1.2); 8.150(1.0); 8.147(1.5); 8.144(1.1); 8.125(6.5); 7.838(1.5); 7.819(2.8); 7.799(1.3); 7.692(1.0); 7.689(1.1); 7.672(2.5); 7.648(1.0); 7.643(1.3); 7.630 (1.3); 7.625(1.8); 7.617(1.3); 7.613(1.3); 7.606(1.3); 7.597(2.4); 7.594(1.4); 7.549(1.4); 7.545(1.4); 7.530(1.6); 7.512(0.7); 7.509(0.6); 3.725(2.3); 3.698 (2.1); 3.523(16.0); 2.671(0.3); 2.524(0.7); 2.511(20.2); 2.506(42.9); 2.502 (57.9); 2.497(41.9); 2.493(20.3); 2.329(0.4); 0.000(0.6) |
| 168 | 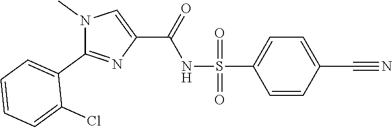 [SMILES: Cn1cc(C(=O)NS(=O)(=O)c2ccc(C#N)cc2)nc1-c1ccccc1Cl] | Example 168: ¹H-NMR(400.0 MHz, d₆-DMSO): d = 8.123(2.2); 8.114(5.5); 8.107(2.2); 8.101(7.9); 8.085(7.4); 8.064(2.0); 7.687(1.3); 7.668(2.7); 7.644 (1.0); 7.640(1.3); 7.626(1.4); 7.622(1.9); 7.614(1.4); 7.609(1.5); 7.602(1.4); 7.594(2.5); 7.546(1.5); 7.543(1.5); 7.526(1.8); 7.509(0.7); 7.506(0.7); 3.807 (1.1); 3.782(1.1); 3.760(1.1); 3.695(0.9); 3.651(0.7); 3.630(0.6); 3.579(0.5); 3.566(0.4); 3.519(16.0); 2.675(0.4); 2.670(0.5); 2.666(0.4); 2.510(32.3); 2.506(66.1); 2.501(88.7); 2.497(66.8); 2.333(0.4); 2.328(0.6); 2.324(0.5); 2.074(9.5); 0.000(0.9); −0.090(0.3) |
| 169 | 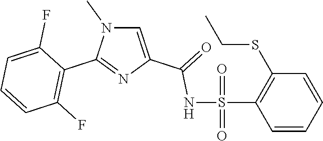 [SMILES: CCSc1ccccc1S(=O)(=O)NC(=O)c1cn(C)c(-c2c(F)cccc2F)n1] | Example 169: ¹H-NMR(400.0 MHz, d₆-DMSO): d = 8.218(5.2); 8.060(2.3); 8.058(2.3); 8.040(2.6); 8.038(2.4); 7.745(0.4); 7.728(1.0); 7.723(1.0); 7.707 (1.8); 7.689(1.0); 7.686(1.1); 7.669(0.5); 7.646(0.7); 7.643(0.7); 7.626(2.0); 7.608(1.8); 7.605(1.7); 7.585(3.0); 7.566(1.4); 7.417(1.4); 7.415(1.3); 7.397 (2.3); 7.380(1.1); 7.377(1.0); 7.356(3.0); 7.336(4.8); 7.316(2.5); 3.565(16.0); 3.386(0.6); 3.352(0.7); 3.336(0.7); 3.053(1.6); 3.035(4.8); 3.016(4.9); 2.998 (1.6); 2.670(0.5); 2.505(63.0); 2.501(82.1); 2.497(62.8); 2.328(0.5); 1.123 (5.2); 1.105(10.8); 1.087(4.9); 0.000(14.9) |
| 170 | 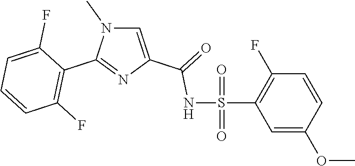 [SMILES: COc1ccc(F)c(S(=O)(=O)NC(=O)c2cn(C)c(-c3c(F)cccc3F)n2)c1] | Example 170: ¹H-NMR(400.0 MHz, d₆-DMSO): d = 8.223(4.3); 7.727(0.8); 7.710(1.3); 7.689(0.9); 7.673(0.4); 7.408(1.2); 7.400(2.3); 7.395(2.1); 7.387 (1.7); 7.375(2.2); 7.358(2.4); 7.352(2.3); 7.338(3.6); 7.317(2.3); 7.306(1.9); 7.298(1.1); 7.283(0.9); 7.275(0.6); 3.825(16.0); 3.750(0.5); 3.642(0.6); 3.576(12.2); 3.416(0.6); 3.396(0.6); 2.670(0.7); 2.501(102.7); 2.497(92.4); 2.328(0.7); 0.000(13.8) |
| 171 | 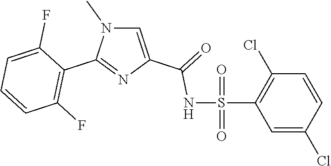 [SMILES: Cn1cc(C(=O)NS(=O)(=O)c2cc(Cl)ccc2Cl)nc1-c1c(F)cccc1F] | Example 171: ¹H-NMR(400.0 MHz, d₆-DMSO): d = 8.241(6.0); 8.080(4.3); 8.073(4.5); 7.790(1.7); 7.784(1.6); 7.769(2.7); 7.763(2.9); 7.747(0.9); 7.742 (0.9); 7.726(1.7); 7.708(1.0); 7.705(1.2); 7.696(5.0); 7.688(0.6); 7.675(3.1); 7.369(3.0); 7.349(4.9); 7.328(2.6); 5.754(0.6); 4.064(0.3); 3.948(0.4); 3.938 (0.4); 3.895(0.4); 3.793(0.5); 3.764(0.6); 3.637(0.3); 3.595(16.0); 2.675(0.6); 2.670(0.8); 2.666(0.6); 2.506(105.8); 2.501(144.7); 2.497(111.2); 2.332(0.7); 2.328(0.9); 2.324(0.7); 0.008(0.9); 0.000(25.3); −0.008(1.1) |
| 172 | 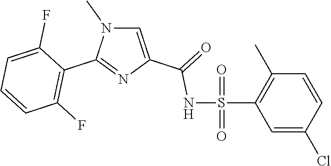 [SMILES: Cc1ccc(Cl)cc1S(=O)(=O)NC(=O)c1cn(C)c(-c2c(F)cccc2F)n1] | Example 172: ¹H-NMR(400.0 MHz, d₆-DMSO): d = 8.196(6.3); 7.981(3.8); 7.975(3.8); 7.749(0.4); 7.732(1.1); 7.711(1.8); 7.694(1.2); 7.673(2.2); 7.668 (1.8); 7.653(2.2); 7.647(2.0); 7.451(3.0); 7.430(2.5); 7.358(2.9); 7.338(4.7); 7.317(2.4); 3.748(0.5); 3.714(0.4); 3.685(0.5); 3.664(0.6); 3.572(16.0); 3.524(0.7); 3.487(0.7); 3.392(0.7); 2.670(0.8); 2.581(15.9); 2.501(121.4); 2.412(0.8); 2.328(0.8); 2.073(0.4); 0.000(15.9) |
| 173 | 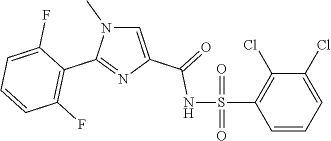 [SMILES: Cn1cc(C(=O)NS(=O)(=O)c2cccc(Cl)c2Cl)nc1-c1c(F)cccc1F] | Example 173: ¹H-NMR(400.0 MHz, d₆-DMSO): d = 8.247(6.0); 8.152(2.6); 8.149(2.1); 8.132(2.9); 8.129(2.2); 7.988(2.6); 7.968(3.0); 7.757(0.4); 7.740 (1.2); 7.719(1.8); 7.701(1.3); 7.680(0.4); 7.649(1.9); 7.629(3.5); 7.609(1.6); 7.364(2.8); 7.344(4.9); 7.324(2.5); 3.801(1.1); 3.765(1.1); 3.729(1.1); 3.589 (16.0); 3.409(0.4); 2.671(0.7); 2.505(97.5); 2.502(105.7); 2.328(0.6); 0.004 (6.7); 0.000(12.4) |
| 174 | 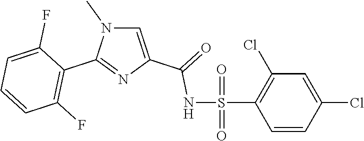 [SMILES: Cn1cc(C(=O)NS(=O)(=O)c2ccc(Cl)cc2Cl)nc1-c1c(F)cccc1F] | Example 174: ¹H-NMR(400.0 MHz, d₆-DMSO): d = 8.248(7.6); 8.148(4.1); 8.126(4.6); 7.870(4.4); 7.865(4.6); 7.754(0.4); 7.737(1.0); 7.732(1.0); 7.718 (3.7); 7.713(3.6); 7.697(3.3); 7.691(2.8); 7.678(0.4); 7.361(3.1); 7.341(4.9); 7.320(2.6); 3.586(16.0); 3.568(0.4); 2.507(24.6); 2.503(34.8); 2.498(27.8); 2.075(3.2); 0.000(7.0) |
| 175 | 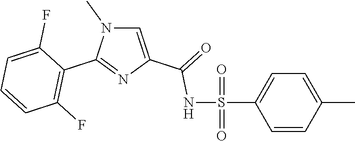 [SMILES: Cc1ccc(S(=O)(=O)NC(=O)c2cn(C)c(-c3c(F)cccc3F)n2)cc1] | Example 175: ¹H-NMR(400.0 MHz, d₆-DMSO): d = 8.153(8.1); 7.887(5.2); 7.866(5.9); 7.740(0.4); 7.723(0.9); 7.718(0.9); 7.702(1.7); 7.685(0.9); 7.681 (1.0); 7.664(0.5); 7.432(4.6); 7.411(4.3); 7.357(0.5); 7.350(3.0); 7.329(4.7); 7.309(2.6); 7.302(0.5); 3.554(16.0); 3.374(0.4); 3.347(0.3); 2.524(0.6); 2.511 (17.8); 2.506(37.3); 2.502(52.0); 2.498(39.4); 2.494(19.8); 2.394(15.4); 2.329 (0.4); 2.075(1.0); 2.074(1.0); 0.000(9.6); −0.007(0.3) |
| 176 | 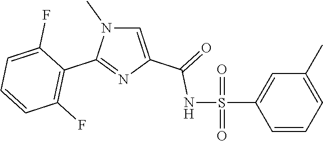 [SMILES: Cc1cccc(S(=O)(=O)NC(=O)c2cn(C)c(-c3c(F)cccc3F)n2)c1] | Example 176: ¹H-NMR(400.0 MHz, d₆-DMSO): d = 8.158(4.9); 7.801(5.1); 7.788(1.8); 7.781(1.4); 7.740(0.4); 7.723(1.0); 7.719(1.0); 7.702(1.7); 7.685 (1.0); 7.681(1.0); 7.664(0.4); 7.514(5.1); 7.501(3.0); 7.480(0.3); 7.351(2.9); 7.331(4.6); 7.310(2.4); 3.555(15.7); 3.339(1.7); 2.670(0.6); 2.506(66.1); 2.502(86.3); 2.497(68.1); 2.405(16.0); 2.328(0.5); 2.073(0.5); 0.000(8.3) |
| 177 | 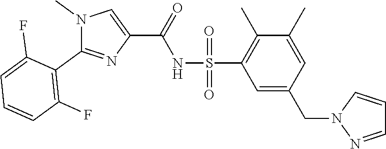 [SMILES: Cc1cc(Cn2cccn2)cc(S(=O)(=O)NC(=O)c2cn(C)c(-c3c(F)cccc3F)n2)c1C] | Example 177: ¹H-NMR(400.0 MHz, d₆-DMSO): d = 8.166(4.6); 7.836(3.9); 7.830(4.1); 7.814(3.6); 7.740(0.4); 7.724(0.9); 7.719(0.9); 7.702(1.7); 7.686 (0.9); 7.682(1.1); 7.665(0.5); 7.469(3.8); 7.465(4.0); 7.351(3.1); 7.331(5.0); 7.322(3.9); 7.311(3.0); 6.278(2.6); 6.273(4.6); 6.268(2.7); 5.370(9.8); 3.563 (16.0); 3.382(1.9); 3.346(2.0); 3.168(0.5); 2.675(0.5); 2.670(0.6); 2.666(0.5); 2.523(1.6); 2.505(69.3); 2.501(97.2); 2.497(76.3); 2.465(14.5); 2.333(0.4); 2.328(0.6); 2.323(0.5); 2.257(14.5); 2.073(3.8); 0.000(7.5) |
| 178 | 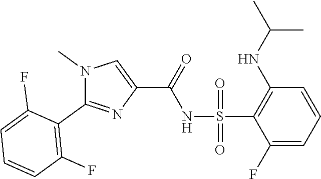 [SMILES: CC(C)Nc1cccc(F)c1S(=O)(=O)NC(=O)c1cn(C)c(-c2c(F)cccc2F)n1] | Example 178: ¹H-NMR(400.0 MHz, d₆-DMSO): d = 8.223(2.9); 7.739(0.4); 7.722(0.8); 7.718(0.8); 7.701(1.5); 7.684(0.8); 7.680(0.9); 7.663(0.4); 7.384 (0.6); 7.363(1.3); 7.350(3.1); 7.330(4.2); 7.310(2.1); 6.646(1.8); 6.624(1.7); 6.405(1.0); 6.385(1.1); 6.377(1.1); 6.357(1.0); 3.779(0.4); 3.765(0.7); 3.749 (0.9); 3.733(0.7); 3.718(0.4); 3.566(13.2); 3.454(0.4); 3.444(0.4); 3.409(0.5); 3.386(0.5); 3.372(0.5); 3.349(0.5); 3.340(0.5); 3.239(0.4); 2.670(0.5); 2.666 (0.4); 2.506(55.8); 2.501(76.9); 2.497(59.2); 2.328(0.5); 2.323(0.3); 2.073 (0.6); 1.215(16.0); 1.199(15.9); 0.000(6.6) |
| 179 | 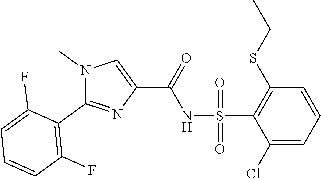 [SMILES: CCSc1cccc(Cl)c1S(=O)(=O)NC(=O)c1cn(C)c(-c2c(F)cccc2F)n1] | Example 179: ¹H-NMR(400.0 MHz, d₆-DMSO): d = 8.225(6.1); 7.745(0.4); 7.725(1.0); 7.707(1.7); 7.686(1.2); 7.669(0.5); 7.529(0.8); 7.509(2.7); 7.490 (3.0); 7.477(3.7); 7.457(1.2); 7.376(2.9); 7.356(4.5); 7.335(5.0); 7.315(2.6); 3.575(16.0); 3.014(1.3); 2.996(4.1); 2.978(4.2); 2.960(1.5); 2.670(0.4); 2.501(60.5); 2.328(0.5); 2.073(1.8); 1.255(4.5); 1.237(9.4); 1.219(4.6); 0.000 (4.2) |
| 180 | 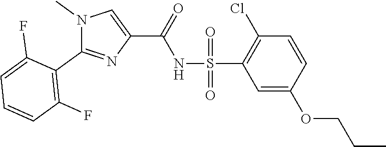 [SMILES: CCCOc1ccc(Cl)c(S(=O)(=O)NC(=O)c2cn(C)c(-c3c(F)cccc3F)n2)c1] | Example 180: ¹H-NMR(400.0 MHz, d₆-DMSO): d = 8.242(4.7); 7.745(0.4); 7.724(1.0); 7.707(1.7); 7.687(1.2); 7.669(0.5); 7.595(3.6); 7.588(4.0); 7.549 (2.9); 7.528(3.4); 7.355(2.8); 7.335(4.8); 7.315(2.5); 7.279(1.7); 7.272(1.9); 7.257(1.6); 7.250(1.6); 4.033(2.9); 4.016(6.0); 4.000(3.2); 3.853(0.3); 3.831 (0.4); 3.784(0.4); 3.753(0.5); 3.683(0.4); 3.633(0.4); 3.578(16.0); 3.398(0.4); 2.670(0.5); 2.501(90.7); 2.498(76.5); 2.328(0.6); 1.806(0.4); 1.788(1.6); 1.771(3.2); 1.753(3.4); 1.736(1.8); 1.719(0.5); 1.012(5.1); 0.993(10.4); 0.975 (4.8); 0.000(6.4) |
| 181 | 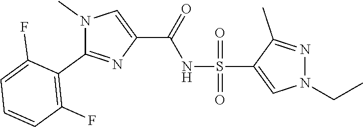 [SMILES: CCn1cc(S(=O)(=O)NC(=O)c2cn(C)c(-c3c(F)cccc3F)n2)c(C)n1] | Example 181: ¹H-NMR(400.0 MHz, d₆-DMSO): d = 8.345(5.3); 8.185(5.4); 7.738(0.3); 7.717(0.9); 7.700(1.6); 7.680(1.1); 7.663(0.4); 7.350(2.5); 7.330 (4.2); 7.310(2.2); 4.136(1.3); 4.118(3.9); 4.100(4.0); 4.082(1.4); 3.564(13.8); 3.322(2.4); 2.502(51.0); 2.334(16.0); 1.367(4.3); 1.349(8.8); 1.331(4.3); 0.000(3.4) |
| 182 | 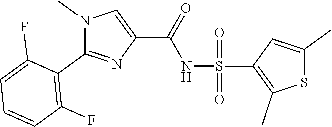 [SMILES: Cc1cc(S(=O)(=O)NC(=O)c2cn(C)c(-c3c(F)cccc3F)n2)c(C)s1] | Example 182: ¹H-NMR(400.0 MHz, d₆-DMSO): d = 8.182(5.4); 7.742(0.4); 7.725(1.0); 7.721(1.0); 7.704(1.8); 7.687(1.1); 7.683(1.1); 7.666(0.4); 7.354 (2.8); 7.334(4.7); 7.313(2.5); 7.017(4.5); 3.568(15.5); 3.324(1.8); 2.670(0.6); 2.634(16.0); 2.505(70.7); 2.501(87.2); 2.497(70.6); 2.369(14.0); 2.328(0.5); 2.073(0.9); 0.000(5.5) |
| 183 | 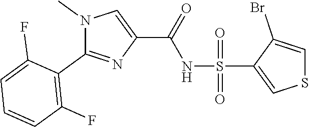 [SMILES: Cn1cc(C(=O)NS(=O)(=O)c2cscc2Br)nc1-c1c(F)cccc1F] | Example 183: ¹H-NMR(400.0 MHz, d₆-DMSO): d = 8.264(5.7); 8.262(5.7); 8.042(3.1); 8.039(3.2); 8.029(3.4); 8.026(3.4); 7.754(0.4); 7.737(1.2); 7.719 (1.8); 7.698(1.4); 7.681(0.5); 7.363(2.7); 7.343(5.0); 7.323(2.6); 7.268(3.4); 7.265(3.4); 7.255(3.5); 7.252(3.5); 4.303(0.3); 4.218(0.4); 4.188(0.5); 4.118 (0.6); 4.099(0.6)4.078(0.6); 4.053(0.6); 4.029(0.6); 3.996(0.6); 3.981(0.6); 3.965(0.6); 3.943(0.6); 3.909(0.6); 3.880(0.6); 3.844(0.6); 3.835(0.6); 3.817 (0.6); 3.813(0.6); 3.767(0.6); 3.633(0.4); 3.592(16.0); 3.563(0.8); 2.668(0.6); 2.502(87.4); 2.498(86.8); 2.330(0.8); 0.000(4.9); −0.003(4.4) |
| 184 | 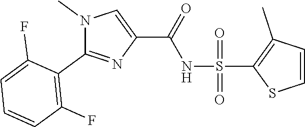 [SMILES: Cc1ccsc1S(=O)(=O)NC(=O)c1cn(C)c(-c2c(F)cccc2F)n1] | Example 184: ¹H-NMR(400.0 MHz, d₆-DMSO): d = 8.202(8.2); 7.887(4.0); 7.874(4.1); 7.743(0.4); 7.726(0.9); 7.722(0.9); 7.705(1.8); 7.688(0.9); 7.684 (1.1); 7.667(0.5); 7.361(0.5); 7.354(3.1); 7.333(4.7); 7.313(2.7); 7.306(0.5); 7.037(3.9); 7.024(3.8); 3.571(16.0); 2.524(0.6); 2.511(11.5); 2.506(24.3); 2.502(34.0); 2.497(26.0); 2.493(12.8); 2.463(20.0); 2.074(0.7); 0.000(3.4) |
| 185 | 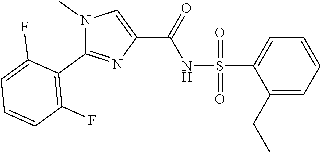 [SMILES: CCc1ccccc1S(=O)(=O)NC(=O)c1cn(C)c(-c2c(F)cccc2F)n1] | Example 185: ¹H-NMR(400.0 MHz, d₆-DMSO): d = 8.172(7.9); 8.027(2.0); 8.025(2.3); 8.008(2.2); 8.005(2.4); 7.740(0.5); 7.724(1.0); 7.719(0.9); 7.707 (0.7); 7.702(1.9); 7.698(0.7); 7.686(0.9); 7.681(1.1); 7.665(0.5); 7.635(0.9); 7.632(1.0); 7.616(1.9); 7.613(2.1); 7.598(1.4); 7.594(1.4); 7.452(2.8); 7.446 (1.9); 7.432(2.2); 7.427(2.4); 7.408(1.1); 7.405(1.0); 7.357(0.5); 7.350(3.4); 7.330(4.6); 7.310(2.8); 7.302(0.5); 5.754(1.1); 3.563(16.0); 3.077(1.3); 3.058(4.0); 3.040(4.1); 3.021(1.3); 2.524(0.7); 2.520(1.0); 2.511(13.5); 2.506(28.6); 2.502(40.0); 2.497(29.3); 2.493(13.6); 1.181(5.1); 1.162(11.3); 1.143(5.0); 0.000(3.2) |
| 186 | 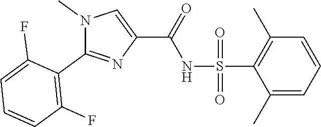 [SMILES: Cc1cccc(C)c1S(=O)(=O)NC(=O)c1cn(C)c(-c2c(F)cccc2F)n1] | Example 186: ¹H-NMR(400.0 MHz, d₆-DMSO): d = 8.165(1.1); 7.721(0.5); 7.717(0.5); 7.700(0.9); 7.683(0.5); 7.679(0.5); 7.407(0.5); 7.388(0.9); 7.370 (0.7); 7.348(1.6); 7.328(2.3); 7.308(1.3); 7.231(2.0); 7.212(1.7); 3.558(7.4); 3.321(4.9); 2.674(16.0); 2.510(44.3); 2.506(75.9); 2.501(98.0); 2.497(74.9); 2.493(42.5); 2.332(0.5); 2.328(0.6); 0.000(7.2); −0.010(0.7) |
| 187 | 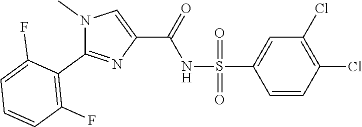 [SMILES: Cn1cc(C(=O)NS(=O)(=O)c2ccc(Cl)c(Cl)c2)nc1-c1c(F)cccc1F] | Example 187: ¹H-NMR(400.0 MHz, d₆-DMSO): d = 8.197(7.1); 8.157(4.3); 8.156(4.5); 7.954(0.4); 7.929(10.4); 7.908(0.4); 7.757(0.4); 7.740(0.9); 7.736(1.0); 7.719(1.7); 7.702(1.0); 7.698(1.1); 7.681(0.4); 7.364(3.0); 7.343 (4.8); 7.323(2.5); 3.575(16.0); 2.507(34.5); 2.503(48.0); 2.499(39.2); 2.330 (0.3); 0.000(3.8) |
| 188 | 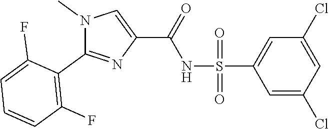 [SMILES: Cn1cc(C(=O)NS(=O)(=O)c2cc(Cl)cc(Cl)c2)nc1-c1c(F)cccc1F] | Example 188: ¹H-NMR(400.0 MHz, d₆-DMSO): d = 8.313(0.3); 8.211(6.3); 8.017(2.0); 8.012(4.1); 8.008(2.6); 7.939(11.2); 7.934(10.3); 7.766(0.4); 7.750(0.9); 7.745(0.9); 7.728(1.8); 7.712(0.9); 7.707(1.1); 7.691(0.5); 7.378 (0.6); 7.372(3.1); 7.351(4.8); 7.331(2.6); 7.325(0.6); 3.842(0.4); 3.795(0.4); 3.761(0.4); 3.737(0.4); 3.696(0.4); 3.681(0.4); 3.661(0.4); 3.586(16.0); 3.533(0.3); 2.675(0.6); 2.671(0.8); 2.666(0.6); 2.524(1.5); 2.510(44.8); 2.506(97.3); 2.501(138.9); 2.497(107.4); 2.492(54.5); 2.333(0.7); 2.328 (0.9); 2.324(0.8); 0.008(0.4); 0.000(13.2); −0.009(0.6) |
| 189 | 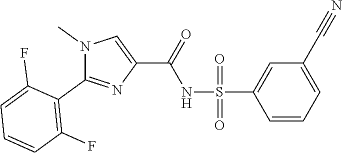 [SMILES: Cn1cc(C(=O)NS(=O)(=O)c2cccc(C#N)c2)nc1-c1c(F)cccc1F] | Example 189: ¹H-NMR(400.0 MHz, d₆-DMSO): d = 8.375(2.2); 8.371(4.1); 8.367(2.5); 8.295(1.4); 8.292(1.9); 8.288(1.4); 8.275(1.6); 8.272(2.0); 8.268 (1.5); 8.195(9.8); 8.178(1.7); 8.175(2.4); 8.172(1.5); 7.873(2.1); 7.853(3.7); 7.833(1.7); 7.756(0.4); 7.740(1.0); 7.735(0.9); 7.718(1.8); 7.701(1.0); 7.697 (1.1); 7.680(0.5); 7.370(0.6); 7.363(3.2); 7.343(4.8); 7.322(2.7); 7.316(0.6); 3.747(0.4); 3.571(16.0); 2.676(0.5); 2.671(0.7); 2.666(0.5); 2.524(1.8); 2.511 (35.0); 2.506(72.6); 2.502(100.6); 2.497(75.5); 2.493(36.9); 2.333(0.4); 2.329(0.6); 2.324(0.5); 0.000(8.5) |
| 190 | 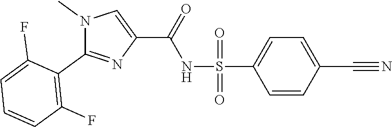 [SMILES: Cn1cc(C(=O)NS(=O)(=O)c2ccc(C#N)cc2)nc1-c1c(F)cccc1F] | Example 190: ¹H-NMR(400.0 MHz, d₆-DMSO): d = 8.184(6.9); 8.151(1.9); 8.146(1.0); 8.135(1.6); 8.129(11.3); 8.119(10.8); 8.102(0.9); 8.097(1.9); 7.755(0.4); 7.738(0.9); 7.733(0.9); 7.717(1.8); 7.700(0.9); 7.695(1.1); 7.679 (0.5); 7.368(0.6); 7.361(3.0); 7.341(4.7); 7.321(2.6); 3.569(16.0); 2.671(0.4); 2.524(0.6); 2.511(20.8); 2.507(44.7); 2.502(63.4); 2.498(49.1); 2.493(25.0); 2.334(0.3); 2.329(0.4); 2.325(0.4); 0.000(5.8) |
| 191 | 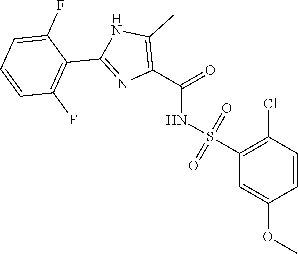 [SMILES: COc1ccc(Cl)c(S(=O)(=O)NC(=O)c2nc(-c3c(F)cccc3F)[nH]c2C)c1] | Example 191: ¹H-NMR(400.0 MHz, d₆-DMSO): d = 13.341(0.4); 7.655(0.6); 7.651(0.6); 7.634(1.2); 7.617(0.7); 7.609(2.9); 7.601(3.0); 7.563(2.3); 7.541 (2.7); 7.332(2.1); 7.311(3.6); 7.291(1.7); 7.270(1.4); 7.263(1.3); 7.248(1.4); 7.241(1.2); 7.065(0.4); 4.269(0.3); 4.256(0.4); 4.031(0.6); 3.852(16.0); 3.733 (0.4); 3.719(0.4); 2.671(0.4); 2.506(48.8); 2.502(66.8); 2.497(51.7); 2.436 (11.8); 2.328(0.4); 2.073(0.6); 0.146(0.3); 0.008(2.6); 0.000(73.4); −0.008 (3.0); −0.150(0.4) |
| 192 | 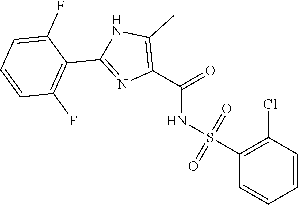 [SMILES: Cc1[nH]c(-c2c(F)cccc2F)nc1C(=O)NS(=O)(=O)c1ccccc1Cl] | Example 192: ¹H-NMR(400.0 MHz, d₆-DMSO): d = 13.292(0.4); 8.157(1.8); 8.154(2.0); 8.137(2.0); 8.134(2.1); 7.707(0.4); 7.703(0.4); 7.686(1.4); 7.683 (1.3); 7.669(2.2); 7.666(2.5); 7.662(2.8); 7.658(3.4); 7.651(1.1); 7.642(1.3); 7.637(0.8); 7.630(1.7); 7.618(1.6); 7.614(1.9); 7.609(1.2); 7.598(1.8); 7.594 (1.6); 7.582(0.9); 7.577(0.8); 7.336(0.5); 7.329(2.9); 7.308(4.9); 7.287(2.4); 7.279(0.5); 7.064(0.5); 4.269(0.3); 3.852(0.6); 3.660(2.9); 3.399(0.4); 2.675 (0.5); 2.671(0.7); 2.667(0.5); 2.524(1.6); 2.511(41.8); 2.506(88.3); 2.502 (123.8); 2.497(93.7); 2.493(46.3); 2.421(16.0); 2.333(0.6); 2.329(0.8); 2.324 (0.6); 0.146(0.6); 0.008(4.7); 0.000(148.2); −0.008(5.6); −0.150(0.7) |
| 193 | 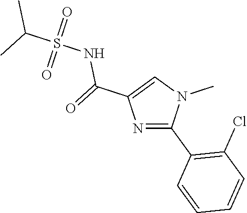 [SMILES: CC(C)S(=O)(=O)NC(=O)c1cn(C)c(-c2ccccc2Cl)n1] | Example 193: ¹H-NMR(400.0 MHz, d₆-DMSO): d = 8.190(5.6); 8.074(0.3); 7.672(1.4); 7.652(2.8); 7.618(1.0); 7.613(1.1); 7.596(1.9); 7.587(1.5); 7.583 (1.6); 7.576(1.4); 7.568(2.8); 7.529(1.8); 7.511(1.9); 7.493(0.7); 3.801(0.5); 3.783(1.2); 3.766(1.6); 3.749(1.2); 3.732(0.5); 3.531(16.0); 3.509(1.3); 3.494(0.6); 3.476(0.6); 3.459(0.4); 3.351(1.0); 3.329(1.0); 3.177(0.4); 2.671 (0.4); 2.505(46.3); 2.501(57.9); 2.329(0.4); 1.311(15.5); 1.294(15.4); 1.136 (0.3); 1.119(0.6); 0.000(20.4) |
| 194 | 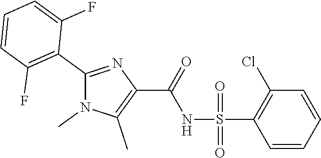 [SMILES: Cc1c(C(=O)NS(=O)(=O)c2ccccc2Cl)nc(-c2c(F)cccc2F)n1C] | Example 194: ¹H-NMR(400.0 MHz, d₆-DMSO): d = 8.155(1.6); 8.151(1.7); 8.135(1.8); 8.131(1.8); 7.756(0.3); 7.739(0.8); 7.735(0.7); 7.718(1.5); 7.696 (1.7); 7.677(1.7); 7.673(1.6); 7.663(2.2); 7.659(2.8); 7.643(1.1); 7.625(1.2); 7.621(1.0); 7.605(1.5); 7.588(0.7); 7.584(0.7); 7.373(0.5); 7.366(2.4); 7.346 (3.7); 7.326(2.0); 7.318(0.4); 3.417(11.6); 2.670(0.5); 2.666(0.3); 2.524(1.0); 2.506(54.7); 2.501(75.5); 2.497(56.6); 2.493(27.6); 2.455(16.0); 2.333(0.4); 2.328(0.5); 2.324(0.4); 2.073(1.1); 0.008(1.7); 0.000(52.2); −0.008(2.1) |
| 195 | 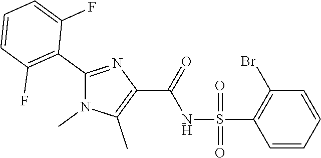 [SMILES: Cc1c(C(=O)NS(=O)(=O)c2ccccc2Br)nc(-c2c(F)cccc2F)n1C] | Example 195: ¹H-NMR(400.0 MHz, d₆-DMSO): d = 8.313(1.0); 8.183(7.0); 8.179(7.4); 8.163(7.8); 8.159(7.7); 7.842(6.7); 7.839(7.1); 7.823(8.1); 7.820 (8.2); 7.754(1.5); 7.737(3.1); 7.733(3.0); 7.716(6.0); 7.700(3.2); 7.695(3.6); 7.678(1.7); 7.664(3.2); 7.648(7.1); 7.645(7.1); 7.629(5.2); 7.626(4.8); 7.602 (4.5); 7.597(4.9); 7.582(6.1); 7.578(6.2); 7.563(2.6); 7.559(2.3); 7.372(2.0); 7.365(10.8); 7.345(16.0); 7.324(9.2); 7.317(1.9); 4.039(0.4); 4.017(0.4); 3.958(0.4); 3.864(0.6); 3.823(1.4); 3.782(0.7); 3.639(0.8); 3.595(1.1); 3.588 (0.9); 3.539(0.9); 3.500(1.5); 3.419(50.1); 3.357(0.8); 3.239(0.8); 3.186(0.5); 3.089(0.4); 3.053(0.3); 2.943(0.4); 2.897(0.5); 2.794(0.4); 2.675(2.0); 2.670 (2.8); 2.666(2.0); 2.524(5.3); 2.510(163.4); 2.506(345.0); 2.501(481.9); |
| | | 2.497(357.3); 2.493(172.0); 2.464(70.2); 2.422(0.8); 2.387(1.1); 2.332(2.3); |
| | | 2.328(3.1); 2.323(2.5); 2.300(0.6); 2.254(0.3); 2.073(3.6); 1.105(0.4); 0.146 |
| | | (1.5); 0.008(11.4); 0.000(343.3); −0.008(11.9); −0.149(1.6) |
| 196 | 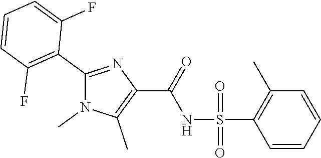 [SMILES: Cc1ccccc1S(=O)(=O)NC(=O)c1nc(-c2c(F)cccc2F)n(C)c1C] | Example 196: ¹H-NMR(400.0 MHz, d₆-DMSO): d = 8.032(2.0); 8.013(2.1); 7.746(0.3); 7.728(0.9); 7.725(0.9); 7.708(1.6); 7.688(1.0); 7.670(0.4); 7.581 (0.8); 7.563(1.9); 7.545(1.3); 7.455(1.2); 7.435(1.9); 7.416(0.9); 7.393(2.1); 7.374(1.8); 7.357(2.6); 7.337(4.3); 7.317(2.2); 3.575(0.5); 3.400(15.3); 3.344(2.4); 3.337(2.4); 3.223(1.1); 2.670(0.8); 2.596(14.7); 2.506(103.0); 2.502(131.2); 2.498(105.5); 2.441(16.0); 2.328(0.8); 2.073(1.0); 0.000(67.8); −0.004(21.7); −0.150(0.3) |
| 197 | 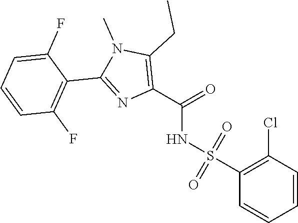 [SMILES: CCc1c(C(=O)NS(=O)(=O)c2ccccc2Cl)nc(-c2c(F)cccc2F)n1C] | Example 197: ¹H-NMR(400.0 MHz, d₆-DMSO): d = 8.312(0.3); 8.156(3.6); 8.152(4.1); 8.136(4.0); 8.133(4.4); 7.757(0.7); 7.739(1.7); 7.735(1.7); 7.718 (3.7); 7.697(4.6); 7.679(3.9); 7.663(5.9); 7.646(2.1); 7.628(2.4); 7.608(3.5); 7.591(1.5); 7.587(1.5); 7.366(5.4); 7.346(8.9); 7.326(4.7); 4.032(0.5); 3.807 (1.0); 3.711(1.1); 3.638(1.2); 3.604(1.1); 3.553(1.0); 3.508(0.9); 3.463(28.3); 3.284(0.5); 2.929(1.9); 2.910(5.6); 2.891(5.7); 2.873(2.0); 2.671(1.0); 2.506 (111.7); 2.502(154.6); 2.498(124.1); 2.333(0.8); 2.329(1.0); 2.073(1.6); 1.083(7.1); 1.065(16.0); 1.046(7.0); 0.146(0.4); 0.000(93.7); −0.150(0.5) |
| 198 | 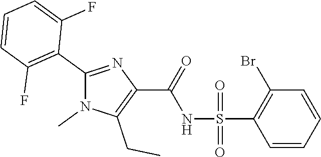 [SMILES: CCc1c(C(=O)NS(=O)(=O)c2ccccc2Br)nc(-c2c(F)cccc2F)n1C] | Example 198: ¹H-NMR(400.0 MHz, d₆-DMSO): d = 8.186(3.6); 8.182(4.0); 8.167(4.0); 8.163(4.1); 7.850(3.8); 7.831(4.3); 7.757(0.8); 7.741(1.7); 7.736 (1.7); 7.719(3.2); 7.703(1.8); 7.699(2.0); 7.681(1.0); 7.673(1.7); 7.655(3.9); 7.637(2.6); 7.635(2.6); 7.611(2.2); 7.607(2.4); 7.592(3.0); 7.588(3.1); 7.573 (1.2); 7.368(5.7); 7.348(8.9); 7.327(4.8); 4.141(0.3); 4.120(0.4); 4.044(0.5); 4.014(0.5); 3.878(0.9); 3.714(1.7); 3.673(1.8); 3.660(1.9); 3.642(2.0); 3.592 (1.8); 3.467(28.3); 3.301(0.6); 3.287(0.7); 3.277(0.5); 3.211(0.4); 2.935(1.8); 2.917(5.5); 2.898(5.6); 2.879(1.9); 2.675(0.8); 2.671(1.1); 2.666(0.8); 2.524 (2.3); 2.506(119.5); 2.502(167.7); 2.498(129.3); 2.328(1.0); 2.324(0.8); 2.073(6.0); 1.088(7.1); 1.070(16.0); 1.051(6.9); 0.146(0.6); 0.008(3.9); |
| | | 0.000(120.7); −0.008(5.3); −0.149(0.6) |
| 199 | 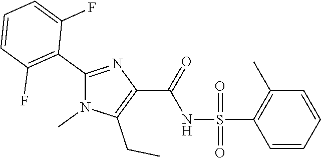 [SMILES: CCc1c(C(=O)NS(=O)(=O)c2ccccc2C)nc(-c2c(F)cccc2F)n1C] | Example 199: ¹H-NMR(400.0 MHz, d₆-DMSO): d = 8.034(2.5); 8.015(2.4); 7.726(1.3); 7.709(1.9); 7.689(1.3); 7.672(0.5); 7.584(1.3); 7.565(2.3); 7.547 (1.5); 7.456(1.7); 7.438(2.3); 7.418(1.4); 7.396(2.7); 7.377(2.5); 7.358(3.4); 7.338(5.0); 7.318(2.6); 3.757(0.3); 3.733(0.4); 3.620(0.7); 3.609(0.7); 3.558 (1.0); 3.446(17.0); 3.339(9.6); 3.018(0.5); 2.897(3.6); 2.879(3.5); 2.861(1.5); 2.670(1.4); 2.596(16.0); 2.502(202.7); 2.391(0.5); 2.328(1.4); 2.073(1.5); 1.079(4.6); 1.061(8.6); 1.042(4.0); 0.147(0.4); 0.000(79.7); −0.150(0.5) |
| 200 | 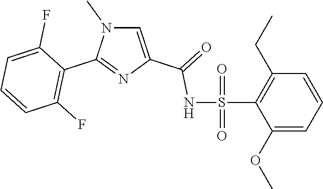 [SMILES: CCc1cccc(OC)c1S(=O)(=O)NC(=O)c1cn(C)c(-c2c(F)cccc2F)n1] | Example 200: ¹H-NMR(400.0 MHz, d₆-DMSO): d = 8.206(4.8); 7.731(0.7); 7.727(0.7); 7.710(1.3); 7.693(0.7); 7.689(0.8); 7.672(0.3); 7.529(1.3); 7.509 (2.4); 7.489(1.5); 7.370(0.4); 7.363(2.3); 7.343(3.5); 7.322(2.0); 7.315(0.4); 7.070(2.0); 7.049(1.7); 6.990(1.9); 6.972(1.8); 3.790(16.0); 3.574(11.6); 3.318(16.1); 3.140(0.9); 3.122(2.8); 3.103(2.9); 3.084(0.9); 2.675(0.4); 2.671 (0.5); 2.666(0.4); 2.524(1.2); 2.510(28.3); 2.506(59.6); 2.501(83.7); 2.497 (63.3); 2.493(31.1); 2.333(0.4); 2.328(0.5); 2.324(0.4); 2.073(0.5); 1.258 (3.3); 1.240(7.5); 1.221(3.3); 0.146(0.5); 0.008(3.5); 0.000(107.3); −0.008 (4.0); −0.150(0.5) |
| 201 | 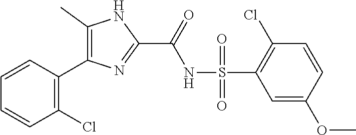 [SMILES: COc1ccc(Cl)c(S(=O)(=O)NC(=O)c2nc(-c3ccccc3Cl)c(C)[nH]2)c1] | Example 201: ¹H-NMR(400.0 MHz, d₆-DMSO): d = 7.642(1.3); 7.630(0.5); 7.623(1.6); 7.619(1.6); 7.607(2.8); 7.599(2.9); 7.559(0.6); 7.554(0.9); 7.549 (1.0); 7.544(1.2); 7.537(1.7); 7.530(2.0); 7.526(1.8); 7.520(1.4); 7.515(0.4); 7.489(1.2); 7.486(1.3); 7.471(0.8); 7.468(1.2); 7.452(0.5); 7.449(0.5); 7.387 (2.7); 7.365(3.2); 7.076(1.6); 7.068(1.5); 7.054(1.4); 7.046(1.4); 3.808(16.0); 2.519(0.5); 2.511(14.0); 2.506(31.2); 2.502(43.3); 2.497(32.0); 2.493(15.9); 2.153(12.5); 2.073(1.7); 0.008(0.6); 0.000(22.5); −0.008(1.1) |
| 202 | 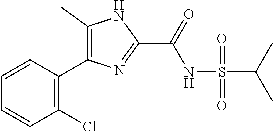 [SMILES: Cc1[nH]c(C(=O)NS(=O)(=O)C(C)C)nc1-c1ccccc1Cl] | Example 202: ¹H-NMR(400.0 MHz, d₆-DMSO): d = 7.604(1.4); 7.601(1.5); 7.584(1.7); 7.582(1.9); 7.522(1.1); 7.516(1.1); 7.504(1.7); 7.499(2.5); 7.481 (1.8); 7.476(1.3); 7.463(1.6); 7.458(2.2); 7.454(1.9); 7.440(1.4); 7.436(1.3); 7.422(0.5); 7.418(0.4); 5.754(3.8); 3.711(0.4); 3.694(1.1); 3.677(1.6); 3.660 (1.1); 3.642(0.4); 2.765(0.3); 2.511(12.5); 2.506(28.2); 2.502(39.0); 2.497 (28.7); 2.493(14.1); 2.164(16.0); 1.273(14.5); 1.256(14.5); 0.000(15.5); −0.009(0.7) |
| 203 | 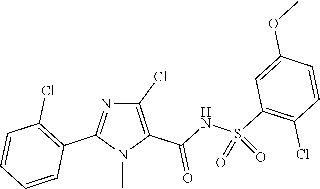 [SMILES: COc1ccc(Cl)c(S(=O)(=O)NC(=O)c2c(Cl)nc(-c3ccccc3Cl)n2C)c1] | Example 203: ¹H-NMR (D6-DMSO): 3.6 (s, 3H), 3.8 (s, 3H), 7.1 (m, 1H), 7.4 (m, 1H), 7.5-7.7 (m, 5H) |
| 204 | 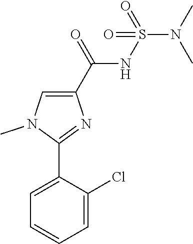 | Example 204: ¹H-NMR(400.0 MHz, d₆-DMSO): d = 8.148(2.4); 7.672(0.5); 7.669(0.4); 7.652(0.9); 7.650(0.8); 7.616(0.4); 7.612(0.5); 7.599(0.5); 7.594 (0.7); 7.585(0.5); 7.580(0.6); 7.574(0.6); 7.566(1.0); 7.561(0.6); 7.529(0.7); 7.525(0.6); 7.509(0.6); 7.506(0.4); 5.753(1.2); 3.528(6.4); 3.324(0.6); 3.289 (0.5); 2.858(16.0); 2.511(4.6); 2.506(9.8); 2.502(13.2); 2.497(9.4); 2.492(4.4) |
| 205 | 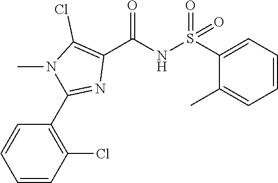 [SMILES: Cc1ccccc1S(=O)(=O)NC(=O)c1nc(-c2ccccc2Cl)n(C)c1Cl] | Example 205: ¹H-NMR(400.0 MHz, d₆-DMSO): d = 8.042(1.9); 8.039(2.1); 8.022(2.1); 8.019(2.2); 7.695(1.5); 7.687(0.6); 7.675(3.3); 7.671(2.5); 7.651 (1.3); 7.647(1.8); 7.634(2.6); 7.630(4.1); 7.616(2.6); 7.612(4.3); 7.595(0.8); 7.592(0.8); 7.576(1.8); 7.573(1.9); 7.560(2.5); 7.557(3.1); 7.542(1.9); 7.540 (2.1); 7.537(1.3); 7.523(0.9); 7.520(0.9); 7.463(1.2); 7.444(1.9); 7.425(0.9); 7.403(2.1); 7.384(1.8); 3.583(0.5); 3.407(24.0); 3.346(5.1); 3.226(1.3); 2.672(0.3); 2.608(16.0); 2.525(0.7); 2.512(17.2); 2.507(35.8); 2.503(47.9); 2.498(35.4); 2.494(17.7); 2.329(0.3); 2.072(0.8); 0.000(9.5); −0.009(0.4) |
| 206 | 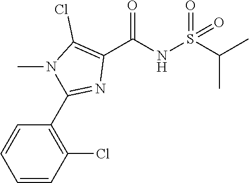 [SMILES: CC(C)S(=O)(=O)NC(=O)c1nc(-c2ccccc2Cl)n(C)c1Cl] | Example 206: ¹H-NMR(400.0 MHz, d₆-DMSO): d = 7.697(1.3); 7.689(0.4); 7.678(2.4); 7.674(2.1); 7.650(1.0); 7.646(1.3); 7.630(2.7); 7.617(2.1); 7.613 (2.9); 7.559(1.5); 7.555(1.5); 7.539(1.7); 7.521(0.7); 7.518(0.7); 3.794(0.4); 3.776(1.2); 3.759(1.6); 3.742(1.2); 3.725(0.5); 3.450(17.0); 3.319(12.6); 2.524(0.8); 2.510(19.5); 2.506(38.7); 2.502(50.5); 2.497(37.1); 1.311(16.0); 1.293(15.8); 0.000(8.4) |
| 207 | 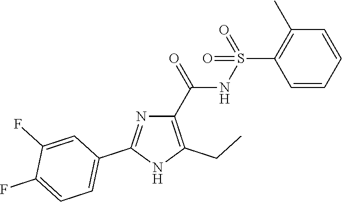 [SMILES: CCc1[nH]c(-c2ccc(F)c(F)c2)nc1C(=O)NS(=O)(=O)c1ccccc1C] | Example 207: ¹H-NMR(400.0 MHz, d₆-DMSO): d = 13.014(1.3); 8.164(0.8); 8.139(1.4); 8.117(0.8); 8.036(1.9); 8.016(2.1); 7.882(1.6); 7.861(1.9); 7.630 (0.5); 7.606(1.4); 7.585(1.9); 7.567(1.7); 7.550(1.1); 7.460(0.9); 7.441(1.5); 7.424(0.9); 7.402(1.7); 7.384(1.4); 3.780(0.3); 3.768(0.3); 3.666(0.5); 3.658 (0.6); 3.341(29.2); 2.975(0.4); 2.928(0.3); 2.846(1.3); 2.827(3.8); 2.808(3.8); 2.789(1.5); 2.674(2.0); 2.670(2.7); 2.666(2.1); 2.630(16.0); 2.523(6.1); 2.505(328.0); 2.501(444.0); 2.497(325.9); 2.332(1.8); 2.328(2.6); 2.324(1.9); 2.301(0.7); 2.073(2.2); 1.145(5.7); 1.127(12.3); 1.108(5.6); 0.000(4.6) |
| 208 | 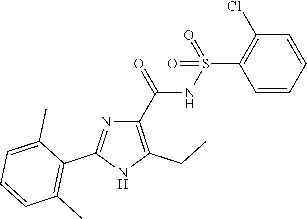 [SMILES: CCc1[nH]c(-c2c(C)cccc2C)nc1C(=O)NS(=O)(=O)c1ccccc1Cl] | Example 208: ¹H-NMR(400.0 MHz, d₆-DMSO): d = 8.133(1.3); 8.084(1.0); 8.066(1.2); 7.514(1.5); 7.505(2.6); 7.496(0.6); 7.485(0.8); 7.476(0.5); 7.466 (0.7); 7.455(0.7); 7.395(0.6); 7.376(1.1); 7.356(0.9); 7.220(2.5); 7.201(2.0); 3.936(0.3); 3.785(0.8); 3.757(0.8); 3.568(0.3); 3.032(0.5); 3.013(1.4); 2.995 (1.5); 2.976(0.5); 2.670(0.4); 2.665(0.3); 2.523(1.0); 2.510(24.1); 2.505 (51.6); 2.501(73.0); 2.496(54.9); 2.492(26.5); 2.332(0.4); 2.327(0.5); 2.323 (0.4); 2.255(0.3); 2.099(16.0); 2.073(2.6); 1.205(2.2); 1.186(4.9); 1.167(2.2); 0.008(0.7); 0.000(22.0); −0.008(0.8) |
| 209 | 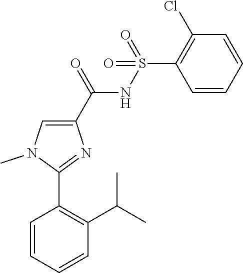 [SMILES: CC(C)c1ccccc1-c1nc(C(=O)NS(=O)(=O)c2ccccc2Cl)cn1C] | Example 209: ¹H-NMR(400.0 MHz, d₆-DMSO): d = 8.141(3.2); 8.123(6.3); 7.651(0.4); 7.635(1.4); 7.631(1.4); 7.615(4.6); 7.610(3.8); 7.595(1.1); 7.578 (1.8); 7.559(3.2); 7.533(3.6); 7.515(1.1); 7.388(1.0); 7.373(3.2); 7.368(3.1); 7.346(1.9); 7.330(0.6); 7.327(0.5); 4.529(0.3); 4.353(0.5); 4.344(0.5); 4.313 (0.5); 4.298(0.5); 3.483(17.8); 2.709(0.4); 2.692(1.0); 2.675(1.6); 2.658(1.0); 2.641(0.4); 2.514(44.5); 2.510(60.0); 2.505(46.3); 2.336(0.4); 2.081(3.6); 1.131(16.0); 1.114(15.9) |
| 210 | 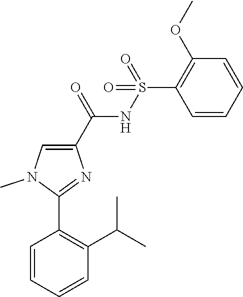 [SMILES: COc1ccccc1S(=O)(=O)NC(=O)c1cn(C)c(-c2ccccc2C(C)C)n1] | Example 210: ¹H-NMR(400.0 MHz, d₆-DMSO): d = 8.103(5.7); 7.907(1.6); 7.904(1.8); 7.888(1.8); 7.884(1.9); 7.690(0.8); 7.686(0.8); 7.668(1.6); 7.651 (1.0); 7.647(1.0); 7.521(1.2); 7.510(3.5); 7.503(4.3); 7.492(0.7); 7.345(0.3); 7.326(1.6); 7.318(3.5); 7.309(3.7); 7.299(0.6); 7.245(2.3); 7.224(2.1); 7.168 (1.2); 7.149(2.2); 7.130(1.1); 3.843(16.0); 3.685(0.4); 3.672(0.8); 3.658(0.4); 3.625(0.4); 3.451(15.3); 3.378(1.0); 3.272(0.7); 3.186(0.4); 2.806(0.4); 2.789 (1.0); 2.772(1.3); 2.755(1.0); 2.738(0.4); 2.670(0.5); 2.666(0.4); 2.506(52.7); 2.501(71.8); 2.497(56.8); 2.328(0.5); 2.324(0.4); 2.073(1.6); 1.483(1.7); 1.124(13.1); 1.107(13.0); 0.000(62.5) |
| 211 | 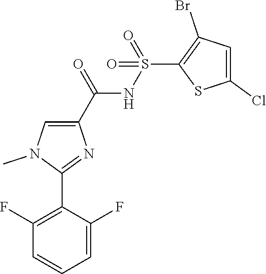 [SMILES: Cn1cc(C(=O)NS(=O)(=O)c2sc(Cl)cc2Br)nc1-c1c(F)cccc1F] | Example 211: ¹H-NMR(400.0 MHz, CD3CN): d = 7.948(6.9); 7.704(0.5); 7.687(1.0); 7.682(1.0); 7.671(0.6); 7.666(2.0); 7.661(0.7); 7.650(1.0); 7.645 (1.2); 7.628(0.6); 7.250(0.4); 7.246(0.6); 7.239(3.3); 7.219(4.6); 7.198(2.9); 7.188(9.4); 3.657(0.4); 3.613(16.0); 2.739(0.6); 2.557(1.2); 2.530(1.3); 2.490(1.4); 2.144(0.6); 2.138(0.7); 2.131(0.5); 1.995(5.0); 1.988(7.9); 1.983 (46.7); 1.976(86.6); 1.970(119.4); 1.964(82.7); 1.958(42.5); 1.811(0.4); 1.805(0.6); 1.799(0.8); 1.792(0.6) |
| 212 | 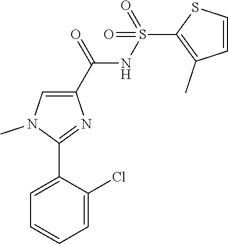 [SMILES: Cc1ccsc1S(=O)(=O)NC(=O)c1cn(C)c(-c2ccccc2Cl)n1] | Example 212: ¹H-NMR(400.0 MHz, d₆-DMSO): d = 8.129(6.1); 7.865(2.9); 7.853(3.0); 7.668(1.3); 7.649(2.7); 7.619(1.0); 7.615(1.3); 7.601(1.4); 7.597 (1.9); 7.582(1.5); 7.577(2.0); 7.564(2.8); 7.560(1.8); 7.528(1.6); 7.525(1.6); 7.508(1.7); 7.490(0.6); 7.488(0.6); 7.027(3.0); 7.015(3.0); 5.754(2.5); 3.512 (16.0); 2.506(33.2); 2.501(43.9); 2.497(32.6); 2.460(15.9); 0.007(0.8); 0.000 (20.1); −0.008(0.9) |
| 213 | 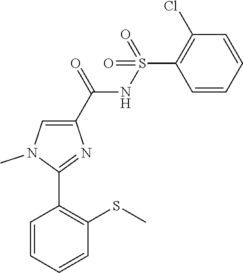 [SMILES: CSc1ccccc1-c1nc(C(=O)NS(=O)(=O)c2ccccc2Cl)cn1C] | Example 213: ¹H-NMR(400.0 MHz, d₆-DMSO): d = 8.132(4.8); 8.119(2.3); 7.884(0.7); 7.668(0.4); 7.648(1.3); 7.625(2.8); 7.621(2.8); 7.605(0.9); 7.585 (1.7); 7.582(1.6); 7.565(3.1); 7.546(1.9); 7.476(2.5); 7.456(1.7); 7.432(0.4); 7.413(1.6); 7.397(2.2); 7.327(1.4); 7.308(2.2); 7.290(1.1); 4.212(0.4); 4.071 (0.6); 4.057(0.6); 4.042(0.6); 4.007(0.6); 3.494(14.1); 3.462(0.5); 3.446(1.8); 2.671(0.4); 2.505(54.8); 2.501(70.4); 2.497(54.5); 2.439(16.0); 2.422(2.2); 2.328(0.4); 2.073(5.2); 1.507(4.8); 0.000(33.2) |
| 214 | 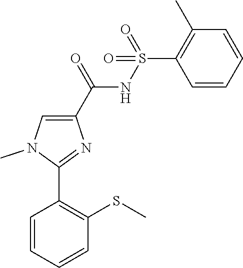 [SMILES: CSc1ccccc1-c1nc(C(=O)NS(=O)(=O)c2ccccc2C)cn1C] | Example 214: ¹H-NMR(400.0 MHz, d₆-DMSO): d = 8.071(4.2); 8.021(1.6); 8.001(1.7); 7.572(0.7); 7.564(0.8); 7.553(1.7); 7.541(1.8); 7.526(1.2); 7.522 (1.3); 7.454(2.3); 7.443(1.2); 7.434(1.8); 7.424(1.7); 7.405(0.8); 7.390(1.8); 7.379(1.4); 7.375(1.9); 7.360(2.1); 7.357(2.0); 7.307(1.3); 7.289(1.9); 7.272 (0.8); 3.761(0.4); 3.644(0.5); 3.538(0.5); 3.522(0.5); 3.517(0.5); 3.470(14.3); 3.446(1.0); 3.410(0.4); 3.291(0.3); 2.675(0.4); 2.670(0.5); 2.666(0.4); 2.605 (11.7); 2.505(54.1); 2.501(75.0); 2.497(58.1); 2.426(16.0); 2.328(0.5); 2.323 (0.4); 2.073(0.9); 1.506(1.2); 0.008(1.8); 0.000(45.9); −0.008(2.6) |
| 215 | 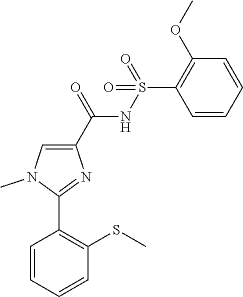 [SMILES: COc1ccccc1S(=O)(=O)NC(=O)c1cn(C)c(-c2ccccc2SC)n1] | Example 215: ¹H-NMR(400.0 MHz, d₆-DMSO): d = 8.112(4.8); 7.904(1.5); 7.900(1.7); 7.884(2.0); 7.881(1.9); 7.689(0.7); 7.685(0.7); 7.667(1.2); 7.650 (0.9); 7.645(0.8); 7.565(0.6); 7.561(0.7); 7.545(1.3); 7.541(1.3); 7.527(1.2); 7.523(1.3); 7.459(2.0); 7.439(1.3); 7.379(1.1); 7.376(1.2); 7.360(1.9); 7.357 (1.8); 7.314(1.2); 7.312(1.2); 7.304(0.4); 7.296(1.7); 7.293(1.8); 7.277(0.7); 7.274(0.9); 7.241(1.9); 7.220(1.7); 7.166(1.0); 7.147(1.8); 7.129(0.9); 7.127 (0.9); 3.850(15.6); 3.734(0.3); 3.478(14.5); 3.462(1.2); 3.446(2.9); 3.359 (2.3); 3.179(0.5); 2.675(0.4); 2.671(0.5); 2.666(0.4); 2.524(1.1); 2.519(1.6); 2.510(26.5); 2.506(56.9); 2.501(80.5); 2.497(60.1); 2.492(28.7); 2.428(16.0); 2.422(2.7); 2.333(0.4); 2.328(0.5); 2.324(0.4); 1.507(5.0); 0.146(0.3); 0.008 (2.6); 0.000(79.9); −0.008(2.7); −0.150(0.3) |
| 216 | 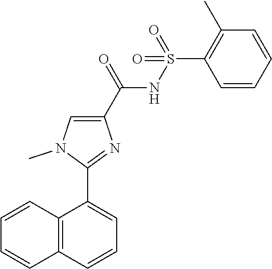 [SMILES: Cc1ccccc1S(=O)(=O)NC(=O)c1cn(C)c(-c2cccc3ccccc23)n1] | Example 216: ¹H-NMR(400.0 MHz, d₆-DMSO): d = 8.181(6.0); 8.133(3.1); 8.115(2.4); 8.101(0.3); 8.093(0.3); 8.060(2.4); 8.043(4.3); 8.024(2.4); 8.000 (1.3); 7.695(1.6); 7.681(4.2); 7.678(3.4); 7.666(5.3); 7.647(3.6); 7.638(1.1); 7.630(2.4); 7.620(1.4); 7.606(2.1); 7.587(3.8); 7.582(4.2); 7.565(3.8); 7.546 (2.0); 7.455(1.4); 7.435(2.2); 7.416(1.1); 7.404(2.4); 7.385(2.0); 3.673(0.4); 3.498(18.6); 3.471(3.7); 2.671(0.6); 2.627(16.0); 2.506(56.6); 2.502(75.4); 2.497(57.6); 2.329(0.4); 2.073(4.7); 1.532(9.6); 0.146(0.4); 0.000(84.1); −0.008(3.8); −0.149(0.4) |
| 217 | 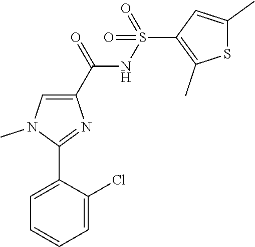 | Example 217: ¹H-NMR(400.0 MHz, d₆-DMSO): d = 8.313(0.6); 8.106(2.8); 7.667(1.2); 7.665(1.2); 7.647(2.5); 7.614(1.0); 7.609(1.1); 7.596(1.3); 7.592 (1.6); 7.576(0.8); 7.572(2.0); 7.567(1.2); 7.552(2.5); 7.548(1.6); 7.525(1.6); 7.522(1.6); 7.506(1.5); 7.488(0.6); 7.485(0.5); 7.017(2.9); 7.015(2.8); 3.583 (0.4); 3.508(16.0); 3.338(11.0); 3.128(0.5); 2.675(1.0); 2.670(1.4); 2.666 (1.0); 2.633(13.0); 2.524(3.2); 2.510(89.7); 2.506(182.9); 2.501(240.8); 2.497(172.1); 2.492(81.7); 2.369(10.3); 2.332(1.1); 2.328(1.5); 2.323(1.1); 0.008(1.4); 0.000(37.2); −0.008(1.3) |
| 218 | 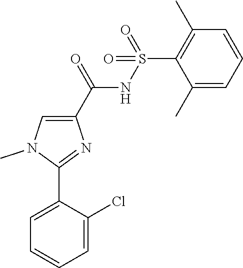 [SMILES: Cc1cccc(C)c1S(=O)(=O)NC(=O)c1cn(C)c(-c2ccccc2Cl)n1] | Example 218: ¹H-NMR(400.0 MHz, d₆-DMSO): d = 8.103 (3.7); 7.664 (0.81); 7.646 (1.54); 7.613 (0.56); 7.609 (0.68); 7.595 (0.78); 7.591 (1.01); 7.575 (0.97); 7.571 (1.32); 7.556 (1.52); 7.551 (1.01); 7.523 (0.90); 7.520 (0.94); 7.503 (1.00); 7.486 (0.34); 7.483 (0.35); 7.409 (0.63); 7.390 (1.30); 7.371 (1.01); 7.233 (2.70); 7.214 (2.11); 3.903 (1.28); 3.499 (9.61); 3.450 (0.38); 3.363 (0.50); 3.323 (0.50); 2.676 (16.00); 2.506 (56.64); 2.501 (78.84); 2.497(60.54); 2.328 (0.44); 0.008 (0.79); −0.000 (23.43); −0.008 (0.88) |
| 219 | 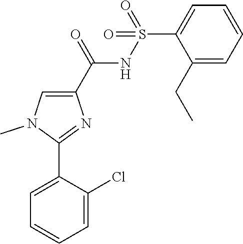 [SMILES: CCc1ccccc1S(=O)(=O)NC(=O)c1cn(C)c(-c2ccccc2Cl)n1] | Example 219: ¹H-NMR(601.6 MHz, d₆-DMSO): d = 19.962(0.6); 8.311(0.5); 8.090(1.0); 8.016(1.3); 8.002(1.4); 7.658(1.4); 7.644(2.2); 7.603(1.8); 7.591 (2.0); 7.577(1.0); 7.561(1.1); 7.552(1.9); 7.515(1.5); 7.503(1.9); 7.492(0.8); 7.440(1.1); 7.428(1.7); 7.416(1.2); 7.404(0.7); 3.501(16.0); 3.327(2.5); 3.070 (1.2); 3.057(3.5); 3.045(3.6); 3.033(1.2); 2.612(1.0); 2.522(1.2); 2.519(1.6); 2.516(1.3); 2.507(49.6); 2.504(112.8); 2.501(161.1); 2.498(117.3); 2.495 (55.4); 2.385(1.2); 1.178(4.5); 1.166(10.0); 1.154(4.6); 0.097(1.4); 0.005 (9.2); 0.000(341.3); −0.006(11.3); −0.100(1.5) |
| 220 | 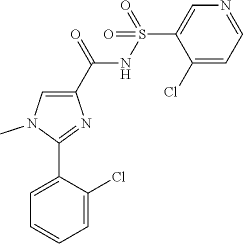 [SMILES: Cn1cc(C(=O)NS(=O)(=O)c2cnccc2Cl)nc1-c1ccccc1Cl] | Example 220: ¹H-NMR(601.6 MHz, d₆-DMSO): d = 19.949(0.4); 8.591 (1.3); 8.586(1.2); 8.494(1.4); 8.491(1.3); 8481(1.4); 8.311(0.3); 8.168(1.2); 7.701(1.2); 7.688(2.3); 7.659(3.0); 7.646(3.8); 7.637(1.4); 7.555(1.2); 7.542(1.8); 7.529(0.8); 3.852(0.4); 3.555(16.0); 3.475(3.0); 2.613(0.9); 2.522(1.4); 2.519(1.6); 2.516(1.6); 2.504(99.9); 2.501(137.5); 2.498(100.0); 2.385(1.1); 0.096(1.1); 0.005(7.1); 0.000(247.5); −0.006(8.7); −0.100(1.2) |
| 221 | 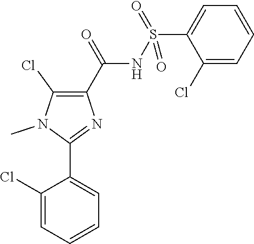 [SMILES: Cn1c(-c2ccccc2Cl)nc(C(=O)NS(=O)(=O)c2ccccc2Cl)c1Cl] | Example 221: ¹H-NMR(400.0 MHz, d₆-DMSO): d = 8.313(0.4); 8.162(5.4); 8.158(6.0); 8.142(6.0); 8.138(6.2); 7.733(1.5); 7.729(1.6); 7.713(4.2); 7.709 (4.1); 7.700(4.3); 7.698(4.9); 7.695(6.1); 7.691(6.4); 7.680(13.8); 7.676 (16.0); 7.659(4.0); 7.655(5.9); 7.651(5.3); 7.642(5.2); 7.638(11.1); 7.634 (9.8); 7.623(8.7); 7.619(13.3); 7.616(10.7); 7.602(2.8); 7.598(2.5); 7.564 (5.0); 7.561(5.1); 7.547(4.1); 7.543(5.2); 7.527(2.4); 7.524(2.3); 3.743(0.4); 3.734(0.4); 3.592(1.0); 3.415(60.2); 3.234(1.3); 3.059(0.4); 3.037(0.4); 2.679(0.5); 2.675(1.0); 2.670(1.4); 2.666(1.0); 2.661(0.5); 2.524(2.5); 2.519 (3.9); 2.510(74.2); 2.506(158.2); 2.501(214.3); 2.497(156.0); 2.492(75.3); 2.337(0.5); 2.333(1.0); 2.328(1.4); 2.323(1.1); 2.319(0.6); 2.073(1.1); 0.146 (1.4); 0.008(10.7); 0.000(343.0); −0.009(12.3); −0.150(1.6) |
| 222 | 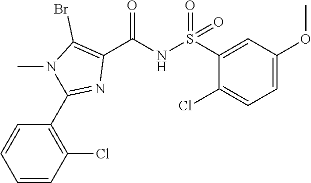 [SMILES: COc1ccc(Cl)c(S(=O)(=O)NC(=O)c2nc(-c3ccccc3Cl)n(C)c2Br)c1] | Example 222: ¹H-NMR(400.0 MHz, d₆-DMSO): d = 7.696(1.3); 7.687(0.3); 7.678(2.1); 7.674(2.1); 7.651(0.8); 7.646(1.2); 7.639(1.2); 7.634(2.0); 7.629 (2.1); 7.620(1.9); 7.616(2.2); 7.612(2.0); 7.608(3.4); 7.600(3.1); 7.584(1.9); 7.562(3.4); 7.540(1.6); 7.524(0.6); 7.521(0.6); 7.301(1.1); 7.293(1.1); 7.279 (1.0); 7.271(1.0); 3.863(16.0); 3.432(14.2); 3.396(0.3); 2.670(0.4); 2.523 (1.0); 2.505(44.2); 2.501(59.5); 2.497(44.6); 2.328(0.4); 0.008(1.8); 0.000 (51.9); −0.008(2.2) |
| 223 | 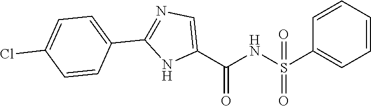 [SMILES: O=C(NS(=O)(=O)c1ccccc1)c1cnc(-c2ccc(Cl)cc2)[nH]1] | Example 223: ¹H-NMR(400.0 MHz, d₆-DMSO): d = 13.355(0.7); 8.319(1.2); 8.061(5.0); 8.035(6.6); 8.025(14.2); 8.021(10.1); 8.015(7.6); 8.012(8.0); 8.007(16.0); 8.003(11.1); 7.974(0.8); 7.958(0.4); 7.952(2.1); 7.948(0.8); 7.936(0.8); 7.931(2.4); 7.925(0.4); 7.889(1.2); 7.884(0.5); 7.872(0.5); 7.867 (1.4); 7.861(0.3); 7.847(0.8); 7.840(7.5); 7.836(8.2); 7.831(3.6); 7.826(3.1); 7.821(8.4); 7.816(9.4); 7.801(0.4); 7.728(1.8); 7.715(1.6); 7.709(5.8); 7.705 (2.3); 7.691(4.7); 7.651(8.6); 7.632(12.1); 7.623(2.6); 7.618(3.1); 7.614 |
| | | (5.2); 7.606(3.7); 7.603(4.3); 7.597(2.8); 7.593(6.6); 7.588(14.6); 7.587 |
| | | (14.5); 7.584(11.6); 7.576(11.3); 7.572(9.9); 7.568(12.6); 7.562(6.6); 7.553 |
| | | (9.9); 7.546(4.0); 7.537(1.3); 7.359(12.3); 6.870(0.7); 6.653(0.3); 5.760(6.6); |
| | | 3.703(0.4); 3.686(0.5); 3.618(0.8); 3.616(0.8); 3.601(1.1); 3.585(1.0); 3.449 |
| | | (2.6); 3.431(3.0); 3.370(3.8); 3.349(3.8); 3.337(3.7); 3.274(2.3); 3.224(1.6); |
| | | 3.206(1.3); 3.187(1.2); 3.047(0.4); 3.034(0.4); 3.018(0.4); 3.005(0.3); 2.944 |
| | | (0.9); 2.784(0.6); 2.681(0.7); 2.676(1.7); 2.672(2.4); 2.667(1.8); 2.663(0.8); |
| | | 2.542(0.4); 2.525(5.8); 2.521(9.1); 2.512(125.0); 2.507(260.0); 2.503(349.2); |
| | | 2.498(260.5); 2.494(132.1); 2.417(0.8); 2.409(0.8); 2.387(0.7); 2.364(0.5); |
| | | 2.339(1.1); 2.334(2.0); 2.329(2.7); 2.325(2.1); 2.321(1.2); 2.183(1.2); 1.958 |
| | | (0.7); 1.760(0.6); 1.655(0.4); 1.640(0.4); 1.627(0.3); 1.557(0.4); 1.532(0.5); |
| | | 1.520(0.5); 1.412(0.5); 1.355(8.1); 1.234(1.1); 1.169(0.6); 1.055(0.5); 0.826 |
| | | (0.4); 0.808(0.6); 0.008(0.6); 0.000(19.8); −0.009(0.9) |
| 224 | 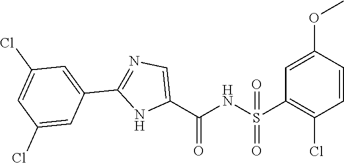 [SMILES: COc1ccc(Cl)c(S(=O)(=O)NC(=O)c2cnc(-c3cc(Cl)cc(Cl)c3)[nH]2)c1] | Example 224: ¹H-NMR(400.0 MHz, d₆-DMSO): d = 8.096(1.5); 8.092(1.4); 7.680(0.5); 7.676(0.7); 7.671(0.4); 7.631(1.2); 7.619(4.6); 7.573(0.5); 7.554 (3.0); 7.532(3.0); 7.479(2.7); 7.471(2.8); 7.195(1.7); 7.187(1.6); 7.173(1.5); 7.165(1.4); 3.858(4.3); 3.816(16.0); 2.507(23.9); 2.503(29.9); 2.499(22.1); 0.008(0.7); 0.000(13.5); −0.008(0.7) |
| 225 | 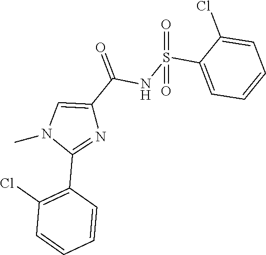 [SMILES: Cn1cc(C(=O)NS(=O)(=O)c2ccccc2Cl)nc1-c1ccccc1Cl] | Example 225: ¹H-NMR(400.0 MHz, d₆-DMSO): d = 8.170(4.7); 8.148(2.0); 8.130(2.1); 7.696(0.6); 7.676(3.0); 7.657(5.1); 7.648(3.7); 7.631(1.7); 7.626 (2.0); 7.623(1.7); 7.605(3.7); 7.592(3.5); 7.577(3.4); 7.532(1.9); 7.513(2.2); 7.495(0.9); 3.521(16.0); 2.670(0.6); 2.501(87.2); 2.328(0.6); 2.074(9.3); 0.000(4.3) |
| 227 | 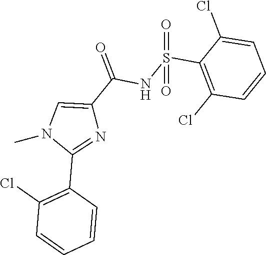 [SMILES: Cn1cc(C(=O)NS(=O)(=O)c2c(Cl)cccc2Cl)nc1-c1ccccc1Cl] | Example 227: ¹H-NMR(400.0 MHz, d₆-DMSO): d = 8.315(0.4); 8.152(1.7); 7.695(1.3); 7.684(0.5); 7.677(2.5); 7.673(2.6); 7.651(1.0); 7.647(1.7); 7.643 (1.6); 7.638(1.5); 7.634(1.3); 7.630(2.5); 7.624(2.5); 7.620(2.0); 7.612(1.6); 7.608(1.0); 7.603(2.2); 7.599(2.6); 7.581(5.3); 7.552(1.6); 7.548(1.6); 7.532 (3.3); 7.515(2.0); 7.511(1.8); 7.492(0.8); 3.543(1.6.0); 2.680(0.4); 2.675 (0.7); 2.671(0.9); 2.666(0.7); 2.524(3.3); 2.510(50.0); 2.506(98.4); 2.501 (128.1); 2.497(91.6); 2.492(43.2); 2.333(0.6); 2.328(0.9); 2.324(0.6); 0.008 (1.0); 0.000(25.8); −0.009(0.7) |
| 228 | 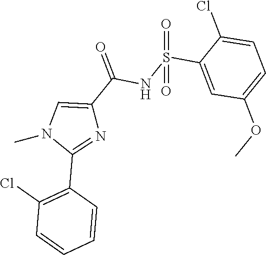 [SMILES: COc1ccc(Cl)c(S(=O)(=O)NC(=O)c2cn(C)c(-c3ccccc3Cl)n2)c1] | Example 228: ¹H-NMR(400.0 MHz, d₆-DMSO): d = 8.161(1.4); 7.679(1.1); 7.659(2.3); 7.629(0.8); 7.625(1.0); 7.611(1.2); 7.601(3.8); 7.593(3.3); 7.584 (2.3); 7.547(1.6); 7.535(1.6); 7.532(1.7); 7.525(2.0); 7.516(1.9); 7.498(0.6); 7.495(0.6); 7.259(0.9); 7.251(1.0); 7.237(0.9); 7.229(0.8); 3.846(16.0); 3.758(0.4); 3.741(0.4); 3.699(0.5); 3.672(0.5); 3.662(0.6); 3.630(0.5); 3.614 (0.5); 3.589(0.5); 3.524(13.3); 3.507(0.8); 3.495(0.6); 3.489(0.6); 3.471(0.5); 3.418(0.4); 3.392(0.4); 3.375(0.5); 3.356(0.4); 3.350(0.4); 3.333(0.4); 2.675 (0.8); 2.670(1.0); 2.666(0.8); 2.524(3.1); 2.506(121.1); 2.501(157.5); 2.497 (116.9); 2.332(0.9); 2.328(1.1); 2.324(0.9); 2.074(0.6); 1.033(0.5); 1.016 (0.9); 0.998(0.4); 0.008(0.9); 0.000(23.0); −0.008(1.0) |
| 229 | 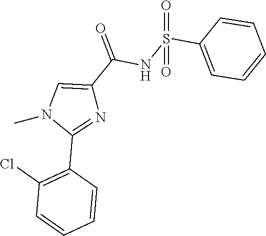 [SMILES: Cn1cc(C(=O)NS(=O)(=O)c2ccccc2)nc1-c1ccccc1Cl] | Example 229: ¹H-NMR(400.0 MHz, d₆-DMSO): d = 8.088(4.0); 8.002(3.1); 7.984(3.6); 7.981(2.7); 7.716(0.5); 7.698(1.7); 7.680(1.4); 7.666(1.3); 7.663 (1.3); 7.642(3.1); 7.639(2.9); 7.619(3.7); 7.610(1.5); 7.601(1.6); 7.597(2.0); 7.592(1.8); 7.577(0.7); 7.572(1.2); 7.568(1.1); 7.563(1.2); 7.548(2.6); 7.544 (1.8); 7.523(1.7); 7.520(1.7); 7.505(1.7); 7.503(1.7); 7.486(0.6); 7.484(0.6); 3.495(16.0); 3.339(0.8); 3.316(0.8); 2.675(0.8); 2.670(1.1); 2.666(0.8); 2.523(3.4); 2.510(64.3); 2.506(124.4); 2.501(161.7); 2.497(117.8); 2.492 (58.1); 2.332(0.8); 2.328(1.1); 2.323(0.8); 2.074(0.5); 0.008(1.0); 0.000 (25.8); −0.009(0.9) |
| 230 | 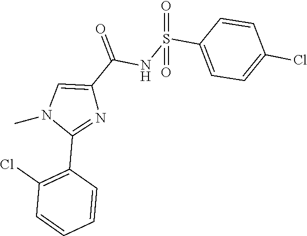 [SMILES: Cn1cc(C(=O)NS(=O)(=O)c2ccc(Cl)cc2)nc1-c1ccccc1Cl] | Example 230: ¹H-NMR(400.0 MHz, d₆-DMSO): d = 8.103(5.8); 8.003(0.6); 7.996(4.6); 7.979(1.7); 7.975(5.4); 7.718(0.8); 7.712(5.3); 7.695(1.6); 7.691 (4.6); 7.670(1.4); 7.652(2.6); 7.650(2.6); 7.623(1.0); 7.618(1.2); 7.605(1.4); 7.601(1.8); 7.585(0.8); 7.580(2.1); 7.575(1.4); 7.561(2.6); 7.556(1.9); 7.530 (1.6); 7.527(1.7); 7.510(1.8); 7.493(0.6); 7.490(0.6); 3.504(16.0); 2.506 (29.5); 2.502(38.9); 2.498(29.2); 2.075(3.6); 0.007(0.7); 0.000(17.1); −0.008(0.9) |
| 231 | 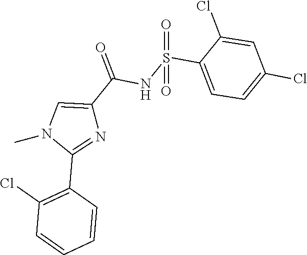 [SMILES: Cn1cc(C(=O)NS(=O)(=O)c2ccc(Cl)cc2Cl)nc1-c1ccccc1Cl] | Example 231: ¹H-NMR(400.0 MHz, d₆-DMSO): d = 8.165(3.6); 8.121(3.2); 8.099(3.6); 7.816(2.5); 7.811(2.7); 7.693(1.4); 7.686(0.5); 7.673(3.7); 7.670 (3.4); 7.654(1.7); 7.649(2.0); 7.643(1.5); 7.626(3.3); 7.608(3.3); 7.548(1.5); 7.545(1.5); 7.528(1.8); 7.511(0.7); 7.507(0.7); 3.537(16.0); 2.675(0.4); 2.671(0.5); 2.666(0.4); 2.524(1.1); 2.510(33.1); 2.506(68.0); 2.501(90.8); 2.497(67.1); 2.493(34.1); 2.333(0.5); 2.328(0.6); 2.324(0.5); 2.074(1.8); 0.008(1.3); 0.000(43.3); −0.008(2.2) |
| 232 | 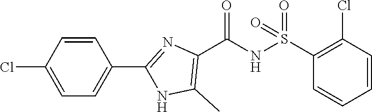 [SMILES: Cc1[nH]c(-c2ccc(Cl)cc2)nc1C(=O)NS(=O)(=O)c1ccccc1Cl] | Example 232: ¹H-NMR(400.0 MHz, d₆-DMSO): d = 8.150(2.3); 8.131(2.5); 8.050(4.8); 8.029(5.6); 7.678(0.5); 7.659(1.5); 7.639(3.7); 7.619(1.2); 7.594 (6.1); 7.573(5.8); 3.884(0.6); 3.186(0.4); 3.120(0.6); 2.801(0.4); 2.782(0.3); 2.671(1.0); 2.506(114.2); 2.502(150.8); 2.498(122.0); 2.424(16.0); 2.328 (1.0); 2.073(0.8); 1.235(2.3); 0.000(22.4) |
| 233 | 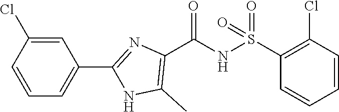 [SMILES: Cc1[nH]c(-c2cccc(Cl)c2)nc1C(=O)NS(=O)(=O)c1ccccc1Cl] | Example 233: ¹H-NMR(400.0 MHz, d₆-DMSO): d = 8.314(0.5); 8.150(4.7); 8.132(2.1); 7.978(2.1); 7.959(2.3); 7.635(2.3); 7.598(1.1); 7.578(1.3); 7.542 (1.0); 7.522(2.0); 7.503(1.6); 7.482(2.1); 7.463(1.0); 5.754(0.8); 3.565(0.8); 3.229(0.3); 3.185(0.7); 2.891(1.0); 2.786(0.4); 2.770(0.4); 2.731(1.0); 2.675 (1.0); 2.671(1.3); 2.551(0.4); 2.541(0.9); 2.506(155.7); 2.502(202.0); 2.497 (154.6); 2.416(16.0); 2.332(1.1); 2.328(1.4); 1.235(0.5); 0.146(1.1); 0.008 (12.8); 0.000(229.6); −0.150(1.1) |
| 234 | 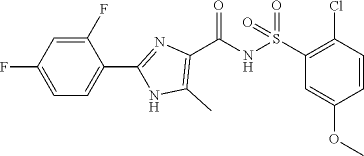 [SMILES: COc1ccc(Cl)c(S(=O)(=O)NC(=O)c2nc(-c3ccc(F)cc3F)[nH]c2C)c1] | Example 234: ¹H-NMR(400.0 MHz, d₆-DMSO): d = 12.953(0.4); 8.203 (0.4); 8.182(0.9); 8.164(0.9); 8.142(0.4); 7.621(2.9); 7.614(2.9); 7.572(2.1); 7.549(2.4); 7.511(0.6); 7.505(0.6); 7.482(1.1); 7.459(0.6); 7.453(0.5); 7.308 (0.6); 7.303(0.6); 7.281(2.2); 7.273(1.3); 7.259(1.5); 7.251(1.0); 3.861 (16.0); 3.773(0.4); 3.760(0.5); 3.749(0.5); 3.591(1.9); 3.443(10.5); 3.338 (0.5); 2.679(0.7); 2.514(92.7); 2.510(119.5); 2.505(88.2); 2.438(9.1); 2.336 (0.8); 2.332(0.6); 2.081(1.3); 0.008(0.3) |
| 235 | 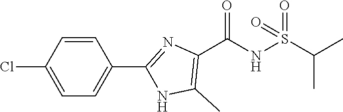 [SMILES: Cc1[nH]c(-c2ccc(Cl)cc2)nc1C(=O)NS(=O)(=O)C(C)C] | Example 235: ¹H-NMR(400.0 MHz, d₆-DMSO): d = 13.122(0.6); 8.037(7.0); 8.015(7.5); 7.568(5.9); 7.547(5.3); 5.754(7.2); 5.320(0.3); 3.806(0.7); 3.790 (1.3); 3.773(1.6); 3.756(1.2); 3.740(0.6); 3.509(0.4); 3.495(0.4); 3.435(0.5); 3.335(0.9); 3.304(0.8); 3.297(0.8); 3.152(0.4); 3.123(1.1); 2.727(0.6); 2.671 (1.0); 2.666(1.1); 2.506(83.0); 2.502(100.3); 2.498(75.2); 2.369(0.5); 2.362 (0.7); 2.329(0.6); 2.324(0.4); 1.319(16.0); 1.302(15.7); 1.254(0.7); 1.235 (2.0); 1.168(0.4); 0.146(0.7); 0.000(144.4); −0.150(0.7) |
| 236 | 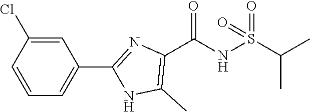 [SMILES: Cc1[nH]c(-c2cccc(Cl)c2)nc1C(=O)NS(=O)(=O)C(C)C] | Example 236: ¹H-NMR(400.0 MHz, d₆-DMSO): d = 13.155(0.7); 8.124(3.6); 7.961(2.2); 7.942(2.3); 7.537(1.3); 7.517(3.1); 7.498(2.2); 7.469(2.6); 7.447 (1.4); 5.754(1.3); 3.818(0.5); 3.801(1.2); 3.784(1.7); 3.767(1.3); 3.750(0.5); 3.173(0.9); 2.520(13.9); 2.503(34.4); 1.328(16.0); 1.311(15.7); 1.254(0.4); 1.236(0.6); 0.000(22.7) |
| 237 | 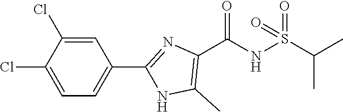 [SMILES: Cc1[nH]c(-c2ccc(Cl)c(Cl)c2)nc1C(=O)NS(=O)(=O)C(C)C] | Example 237: ¹H-NMR(400.0 MHz, d₆-DMSO): d = 13.210(1.4); 8.312(0.5); 8.300(3.2); 8.295(3.3); 7.983(1.9); 7.978(1.8); 7.961(2.2); 7.957(2.2); 7.785 (3.5); 7.764(2.9); 3.817(0.5); 3.800(1.2); 3.783(1.6); 3.766(1.2); 3.749(0.5); 3.318(2.5); 2.675(0.5); 2.671(0.7); 2.520(18.0); 2.506(76.9); 2.502(98.4); 2.497(75.3); 2.333(0.5); 2.329(0.6); 2.073(0.8); 1.328(16.0); 1.311(15.8); 0.000(8.7) |
| 238 | 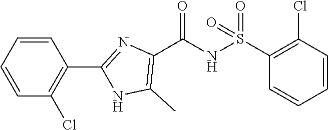 [SMILES: Cc1[nH]c(-c2ccccc2Cl)nc1C(=O)NS(=O)(=O)c1ccccc1Cl] | Example 238: ¹H-NMR(400.0 MHz, d₆-DMSO): d = 8.313(0.4); 8.222(0.4); 8.203(0.4); 8.133(2.1); 8.114(2.2); 7.814(1.8); 7.809(1.7); 7.796(2.1); 7.791 (2.1); 7.650(0.5); 7.628(2.9); 7.624(3.0); 7.609(5.4); 7.575(1.3); 7.556(1.6); 7.539(1.5); 7.536(1.5); 7.522(1.9); 7.517(2.0); 7.502(2.8); 7.498(2.8); 7.483 (2.0); 7.464(0.8); 6.987(0.4); 6.968(0.4); 5.753(1.2); 3.709(1.5); 3.184(4.3); 3.040(0.4); 3.023(0.3); 3.008(0.3); 2.991(0.3); 2.761(2.0); 2.671(1.1); 2.506 (129.7); 2.501(168.0); 2.497(132.4); 2.431(16.0); 2.328(1.1); 1.005(0.3); 0.988(0.6); 0.146(0.8); 0.000(162.6); −0.150(0.8) |
| 239 | 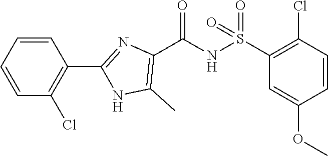 [SMILES: COc1ccc(Cl)c(S(=O)(=O)NC(=O)c2nc(-c3ccccc3Cl)[nH]c2C)c1] | Example 239: ¹H-NMR(400.0 MHz, d₆-DMSO): d = 7.814(1.5); 7.810(1.5); 7.796(1.7); 7.791(1.6); 7.640(1.7); 7.621(2.3); 7.607(2.9); 7.599(2.9); 7.556 (0.8); 7.542(1.7); 7.537(1.8); 7.531(2.3); 7.523(1.5); 7.510(3.2); 7.496(1.9); 7.477(0.7); 7.233(1.2); 7.226(1.2); 7.211(1.1); 7.204(1.0); 5.753(1.8); 4.050 (0.6); 4.035(0.6); 4.024(0.7); 3.942(0.9); 3.897(1.0); 3.845(16.0); 3.730 (0.6); 3.661(0.4); 2.670(0.6); 2.501(100.2); 2.452(10.9); 2.437(1.5); 2.328 (0.7); 0.007(2.4); 0.000(39.6) |
| 240 | 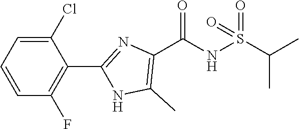 [SMILES: Cc1[nH]c(-c2c(F)cccc2Cl)nc1C(=O)NS(=O)(=O)C(C)C] | Example 240: ¹H-NMR(400.0 MHz, d₆-DMSO): d = 13.117(1.1); 7.632(0.6); 7.616(0.8); 7.611(1.5); 7.596(1.6); 7.591(1.3); 7.575(1.1); 7.517(2.7); 7.497 (1.8); 7.435(1.2); 7.413(2.1); 7.392(1.0); 5.753(1.2); 3.800(0.4); 3.783(1.1); 3.766(1.5); 3.749(1.1); 3.732(0.5); 3.328(1.2); 2.675(0.5); 2.670(0.6); 2.666 (0.5); 2.510(35.3); 2.506(63.7); 2.501(85.0); 2.497(65.1); 2.492(33.9); 2.333 (0.4); 2.328(0.5); 2.324(0.4); 1.311(16.0); 1.294(15.9); 0.008(2.2); 0.000 (51.2); −0.008(2.2) |
| Ex- | | |
| am- | | |
| ple | | |
| No. | Structure | NMR |
| 241 | 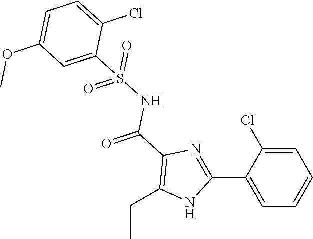 [SMILES: CCc1[nH]c(-c2ccccc2Cl)nc1C(=O)NS(=O)(=O)c1cc(OC)ccc1Cl] | Example 241: ¹H-NMR(400.0 MHz, d₆-DMSO): δ = 13.332(0.4); 7.798(1.7); 7.78(1.92); 7.652(1.67); 7.633(2.54); 7.606(2.8); 7.598(3.02); 7.572(0.85); 7.569(0.86); 7.554(1.76); 7.551(1.65); 7.529 (2.73); 7.522(2.05); 7.506(3.37); 7.484(0.82); 7.228(1.27); 7.222(1.3); 7.206(1.17); 7.199(1.14); 4.107(0.36); 4.066(0.39); 4.023(0.52); 3.992 (0.48); 3.91(0.61); 3.844(16); 2.937(0.79); 2.918(2.28); 2.899(2.4); 2.881 (0.92); 2.501(49.25); 2.073(0.98); 1.175(3.39); 1.156(7.11); 1.137(3.46); 0(15.29) |
| 242 | 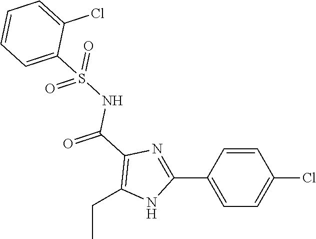 [SMILES: CCc1[nH]c(-c2ccc(Cl)cc2)nc1C(=O)NS(=O)(=O)c1ccccc1Cl] | Example 242: ¹H-NMR(400.0 MHz, d₆-DMSO): δ = 13.22(0.35); 8.161(3.2); 8.157(3.48); 8.141(3.51); 8.137(3.64); 8.072(6.78); 8.05(7.56); 7.917(0.4); 7.895(0.45); 7.691(0.68); 7.687(0.71); 7.671(2.37); 7.668(2.27); 7.654(3.7); 7.65(5.78); 7.644(5.84); 7.629(1.85); 7.624(1.03); 7.611(3.64); 7.605(11.65); 7.588(5.16); 7.583(10.03); 7.574(1.99); 7.569 (1.48); 7.565(0.77); 7.544(0.46); 7.378(1.23); 7.357(1.09); 7.347(0.51); 7.272(0.87); 7.25(0.58); 4.464(0.68); 4.449(0.7); 4.242(0.78); 4.215(1.47); 4.2(1.46); 2.887(1.35); 2.868(4.23); 2.85(4.38); 2.831(1.54); 2.673(0.33); 2.526(0.46); 2.512(20.52); 2.508(43.8); 2.504(59.31); 2.499(43.79); 2.495 (21.86); 2.33(0.42); 2.326(0.33); 2.074(2.93); 1.164(7.17); 1.145(16); 1.126(7.19); 0.008(0.82); 0(30.1); −0.008(1.29) |
| 243 | 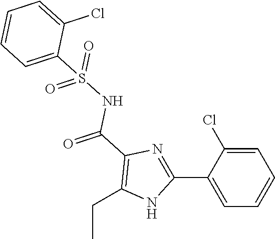 [SMILES: CCc1[nH]c(-c2ccccc2Cl)nc1C(=O)NS(=O)(=O)c1ccccc1Cl] | Example 243: ¹H-NMR(400.0 MHz, d₆-DMSO): δ = 13.254(0.55); 8.149(2.84); 8.145(3.08); 8.129(3.05); 8.126(3.2); 7.806(3.13); 7.801(2.98); 7.787(4.08); 7.782(3.78); 7.674(0.52); 7.67(0.55); 7.654(2.14); 7.65(4.59); 7.646(4.02); 7.634(6.67); 7.63(9.15); 7.626(6.17); 7.616(1.61); 7.595(2.14); 7.59(1.67); 7.575(2.45); 7.57(2.08); 7.568(2.28); 7.563(2.13); 7.559(1.57); 7.554(1.39); 7.549(3.69); 7.544(3.24); 7.53(2.61); 7.525(2.38); 7.52(3.21); 7.516(3.17); 7.501(3.82); 7.497(3.78); 7.482(1.42); 7.479(1.29); 4.743(0.33); 4.672(0.42); 4.645(0.44); 4.638(0.45); 4.621(0.46); 4.617(0.47); 4.556(0.49); 4.514(0.49); 4.486(0.48); 4.403(0.42); 4.386(0.4); 4.361(0.38); 2.916(1.22); 2.897(3.76); 2.878(3.87); 2.86(1.34); 2.525(0.57); 2.52(0.92); 2.511(19.56); 2.507(42.48); 2.502(57.74); 2.498(41.44); 2.493(19.52); 2.329 (0.35); 1.162(7.1); 1.144(16); 1.125(6.99); 0.146(0.38); 0.008(2.99); 0 (101.72); −0.009(3.55); −0.15(0.41) |
| 244 | 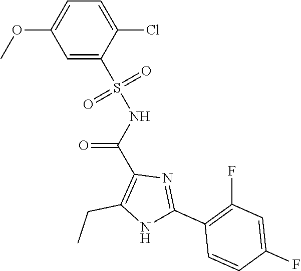 [SMILES: CCc1[nH]c(-c2ccc(F)cc2F)nc1C(=O)NS(=O)(=O)c1cc(OC)ccc1Cl] | Example 244: ¹H-NMR(400.0 MHz, d₆-DMSO): δ = 8.167(0.56); 8.15(0.75); 8.145(1.21); 8.129(1.2); 8.124(0.76); 8.107(0.58); 7.614(2.92); 7.606(3.02); 7.563(2.17); 7.541(2.53); 7.511(0.61); 7.505 (0.62); 7.482(1.13); 7.477(0.79); 7.459(0.61); 7.454(0.61); 7.306(0.64); 7.3 (0.64); 7.284(1.21); 7.279(1.21); 7.27(1.45); 7.262(1.83); 7.248(1.17); 7.24 (1.11); 3.854(16); 2.897(0.6); 2.88(1.72); 2.861(1.75); 2.843(0.64); 2.508 (20.15); 2.504(26.41); 2.499(19.55); 2.075(0.89); 1.142(3.19); 1.123(6.87); 1.104(3.11); 0.007(0.66); 0(14.15) |
| 245 | 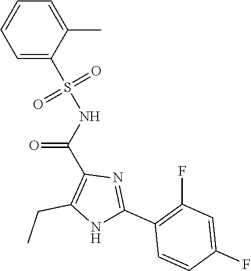 [SMILES: CCc1[nH]c(-c2ccc(F)cc2F)nc1C(=O)NS(=O)(=O)c1ccccc1C] | Example 245: ¹H-NMR(400.0 MHz, d₆-DMSO): δ = 8.133(1.71); 8.023(1.58); 8.003(1.8); 7.536(0.81); 7.468(0.88); 7.444(1.87); 7.416(2); 7.377(1.34); 7.276(1); 7.255(1.61); 7.237(0.94); 3.409(46.89); 3.178(1.5); 3.006(0.49); 2.96(0.51); 2.942(0.86); 2.922(0.84); 2.903(0.45); 2.857(1.47); 2.838(4.18); 2.82(4.31); 2.801(1.57); 2.757(0.75); 2.75(0.67); 2.741(0.5); 2.721(0.41); 2.71(0.8); 2.672(0.95); 2.62(16); 2.597(0.68); 2.507(99.41); 2.503(133.18); 2.498(98.34); 2.329(0.94); 2.086(9.71); 1.91 (0.36); 1.299(0.34); 1.246(0.9); 1.226(1.61); 1.208(0.83); 1.118(5.32); 1.099(11.28); 1.08(5.15); 1.006(0.41); 0.988(0.39); 0(26.9); −0.008(0.99) |
| 246 | 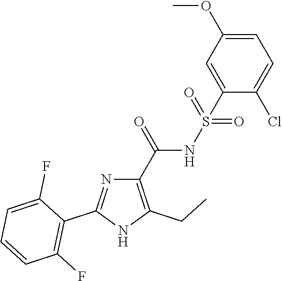 [SMILES: CCc1[nH]c(-c2c(F)cccc2F)nc1C(=O)NS(=O)(=O)c1cc(OC)ccc1Cl] | Example 246: ¹H-NMR(400.0 MHz, d₆-DMSO): δ = 13.338(0.44); 7.665(0.62); 7.661(0.66); 7.644(1.19); 7.624(0.8); 7.607(3.06); 7.599(3); 7.563(2.35); 7.542(2.72); 7.338(2.11); 7.317(3.71); 7.296(1.82); 7.269(1.41); 7.261(1.41); 7.247(1.25); 7.239(1.23); 4.046(1.04); 4.031(1.1); 3.877(0.41); 3.852(16); 2.908(0.79); 2.89(2.47); 2.871(2.54); 2.852(0.88); 2.506(27.49); 2.502(37.22); 2.498(30.28); 1.16(3.48); 1.141(7.28); 1.122 (3.42); 0(8.43) |
| 247 | 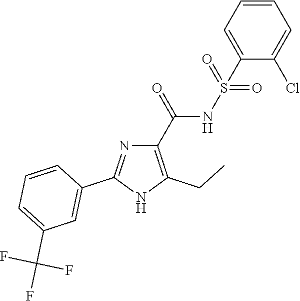 [SMILES: CCc1[nH]c(-c2cccc(C(F)(F)F)c2)nc1C(=O)NS(=O)(=O)c1ccccc1Cl] | Example 247: ¹H-NMR(400.0 MHz, d₆-DMSO): δ = 13.288(0.75); 8.43(5.69); 8.359(3.32); 8.34(3.56); 8.173(3.87); 8.171(3.85); 8.153(4.3); 7.807(1.99); 7.788(4.97); 7.771(3.62); 7.752(4.02); 7.733(1.45); 7.712(0.93); 7.692(3.09); 7.67(5.74); 7.666(6.37); 7.647(2.14); 7.629(2.7); 7.61(3.67); 7.592(1.66); 3.647(12.15); 2.892(2); 2.873(5.84); 2.854(5.97); 2.836(2.12); 2.676(0.43); 2.51(55.57); 2.506(64.1); 2.333(0.39); 2.075 (1.81); 1.172(7.69); 1.153(16); 1.134(7.44); 0(3.42) |
| 248 | 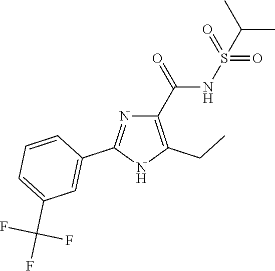 [SMILES: CCc1[nH]c(-c2cccc(C(F)(F)F)c2)nc1C(=O)NS(=O)(=O)C(C)C] | Example 248: ¹H-NMR(400.0 MHz, d₆-DMSO): δ = 13.176(1.28); 8.414(4.08); 8.337(2.18); 8.318(2.41); 7.78(1.07); 7.76(3.7); 7.754(3.27); 7.733(2.41); 7.714(0.82); 3.832(0.5); 3.815(1.16); 3.798(1.57); 3.78(1.23); 3.764(0.55); 3.329(3.64); 3.176(1.28); 2.997(1.15); 2.978(3.04); 2.96(3.12); 2.94(1.22); 2.671(0.48); 2.506(69.09); 2.502(82.68); 2.498 (62.04); 2.329(0.51); 1.332(16); 1.315(15.96); 1.259(4.29); 1.24(8.71); 1.222(4.16); 0(1.13) |
| 249 | 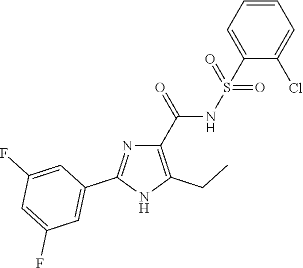 [SMILES: CCc1[nH]c(-c2cc(F)cc(F)c2)nc1C(=O)NS(=O)(=O)c1ccccc1Cl] | Example 249: ¹H-NMR(400.0 MHz, d₆-DMSO): δ = 13.152(0.91); 8.172(3.22); 8.169(3.33); 8.153(3.55); 8.149(3.51); 7.816 (3.63); 7.799(3.61); 7.719(0.73); 7.702(2.39); 7.685(3.2); 7.682(3.23); 7.672(5.06); 7.656(1.79); 7.635(2.13); 7.632(1.78); 7.616(2.89); 7.598 (1.32); 7.594(1.13); 7.342(0.8); 7.336(1.27); 7.318(1.63); 7.313(2.5); 7.295(0.87); 7.29(1.26); 4.033(0.5); 3.885(0.73); 3.792(0.87); 3.737(0.88); 3.698(0.85); 3.692(0.85); 3.37(0.36); 3.186(0.35); 2.861(1.81); 2.843(5.48); 2.824(5.58); 2.805(1.9); 2.676(0.52); 2.672(0.64); 2.507(75.64); 2.503 (97.18); 2.499(69.71); 2.334(0.45); 2.33(0.58); 2.075(10.81); 1.154(7.42); 1.135(16); 1.116(7.18); 0(1.36) |
| 250 | 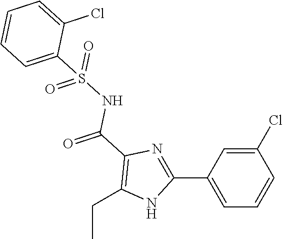 [SMILES: CCc1[nH]c(-c2cccc(Cl)c2)nc1C(=O)NS(=O)(=O)c1ccccc1Cl] | Example 250: ¹H-NMR(400.0 MHz, d₆-DMSO): δ = 13.19(0.64); 8.166(5.6); 8.162(4.55); 8.146(3.58); 8.143(3.6); 8.006(2.99); 7.991(2.09); 7.987(3.23); 7.984(2.04); 7.706(0.72); 7.702(0.73); 7.686(2.35); 7.682(2.24); 7.669(3.56); 7.662(4.9); 7.658(5.57); 7.642(1.83); 7.638(0.97); 7.623(2.42); 7.618(2.01); 7.603(2.77); 7.598(2.11); 7.586(1.53); 7.581(1.34); 7.56(2.07); 7.54(5.21); 7.521(4.31); 7.509(2.94); 7.506(4.03); 7.501(3.12); 7.489(1.35); 7.484(1.69); 7.481(1.19); 4.335(0.34); 4.31(0.37); 4.094(0.56); 4.072(0.55); 4.059(0.55); 4.024(0.53); 4.012(0.52); 3.921(0.42); 3.882(0.38); 3.84(0.33); 2.876(1.31); 2.857(4.09); 2.838(4.21); 2.82(1.45); 2.672(0.44); 2.525(0.63); 2.521(1.03); 2.512(25.18); 2.507(55.8); 2.503(79.94); 2.498 (60.07); 2.494(29.01); 2.334(0.41); 2.33(0.55); 2.325(0.41); 2.075(3.71); 1.159(6.95); 1.14(16); 1.121(7.01); 0.008(0.8); 0(31.61); −0.008(1.18) |
| 251 | 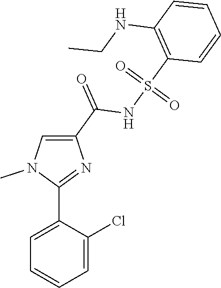 [SMILES: CCNc1ccccc1S(=O)(=O)NC(=O)c1cn(C)c(-c2ccccc2Cl)n1] | Example 251: ¹H-NMR(400.0 MHz, d₆-DMSO): δ = 8.048(3.31); 7.762(1.06); 7.758(1.13); 7.742(1.16); 7.738(1.13); 7.668(0.77); 7.666(0.76); 7.648(1.53); 7.646(1.42); 7.616(0.65); 7.612(0.75); 7.598(0.85); 7.594(1.11); 7.582(0.77); 7.578(0.96); 7.574(0.9); 7.563(1.63); 7.559(0.93); 7.527(0.99); 7.524(0.96); 7.508(0.97); 7.506(0.95); 7.49(0.39); 7.487(0.37); 7.423(0.49); 7.419(0.51); 7.405(0.63); 7.401(0.99); 7.398(0.65); 7.384(0.58); 7.38(0.56); 6.774(1.18); 6.753(1.1); 6.678(0.66); 6.676(0.68); 6.658(1.23); 6.64(0.64); 6.638(0.61); 3.498(9.71); 3.218(0.72); 3.2(1.91); 3.182(1.92); 3.164(0.7); 2.524(0.35); 2.51(9.09); 2.506(19.01); 2.501(26.46); 2.497(19.8); 2.492(9.55); 2.086(16); 1.175(2.68); 1.157(5.82); 1.14(2.64) |
| 252 | 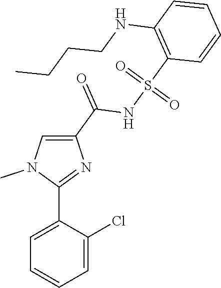 [SMILES: CCCCNc1ccccc1S(=O)(=O)NC(=O)c1cn(C)c(-c2ccccc2Cl)n1] | Example 252: ¹H-NMR(400.0 MHz, d₆-DMSO): δ = 8.036(2.79); 7.751(0.96); 7.747(1.08); 7.731(1.04); 7.727(1.1); 7.667(0.83); 7.647(1.56); 7.616(0.56); 7.611(0.67); 7.598(0.77); 7.594(1.01); 7.578 (0.45); 7.573(1.09); 7.568(0.74); 7.553(1.55); 7.549(1.08); 7.527(0.93); 7.524(1.04); 7.507(1); 7.49(0.32); 7.488(0.36); 7.405(0.48); 7.388(0.93); 7.37(0.53); 7.366(0.53); 6.772(1.19); 6.751(1.1); 6.663(0.69); 6.645(1.19); 6.625(0.63); 3.497(8.76); 3.173(1); 3.155(1.78); 3.138(0.97); 2.505(18.85); 2.501(25.87); 2.496(20.33); 2.085(16); 1.54(0.84); 1.522(1.2); 1.503(0.93); 1.485(0.39); 1.348(0.74); 1.329(1.17); 1.31(1.14); 1.292(0.66); 0.863(2.57); 0.845(5.11); 0.826(2.23) |
| 253 | 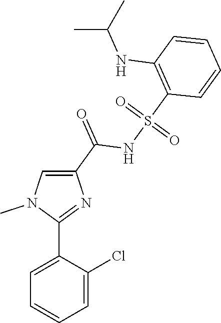 [SMILES: CC(C)Nc1ccccc1S(=O)(=O)NC(=O)c1cn(C)c(-c2ccccc2Cl)n1] | Example 253: ¹H-NMR(600.1 MHz, d₆-DMSO): δ = 8.062(1.4); 7.755(0.51); 7.752(0.53); 7.742(0.55); 7.739(0.53); 7.662(0.42); 7.66(0.43); 7.648(0.68); 7.647(0.64); 7.604(0.36); 7.595(0.43); 7.594(0.33); 7.592(0.5); 7.579(0.34); 7.566(0.37); 7.564(0.38); 7.554(0.68); 7.551(0.53); 7.519(0.45); 7.517(0.45); 7.507(0.55); 7.505(0.52); 7.388(0.47); 6.81(0.54); 6.796(0.52); 6.642(0.59); 3.496(4.84); 2.508(1.43); 2.505(3.14); 2.502 (4.36); 2.499(3.17); 2.496(1.5); 2.086(16); 1.168(5.16); 1.157(5.15) |
| 254 | 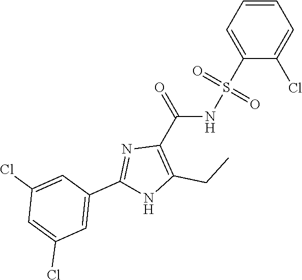 [SMILES: CCc1[nH]c(-c2cc(Cl)cc(Cl)c2)nc1C(=O)NS(=O)(=O)c1ccccc1Cl] | Example 254: ¹H-NMR(400.0 MHz, d₆-DMSO): δ = 13.17(0.64); 8.321(0.34); 8.172(1.34); 8.153(4.96); 8.05(0.36); 8.046(0.34); 7.706(0.76); 7.676(1.68); 7.662(2.53); 7.638(0.75); 7.618(1.02); 7.603 (0.48); 3.621(0.62); 3.393(3.25); 3.124(0.49); 3.097(0.43); 3.003(0.35); 2.985(0.32); 2.863(0.77); 2.844(2.1); 2.825(2.14); 2.807(0.77); 2.678(1.08); 2.513(137.86); 2.509(172.86); 2.505(131.68); 2.336(1.05); 2.081(16); 1.219(0.42); 1.202(0.39); 1.184(0.46); 1.156(2.67); 1.138(5.66); 1.119(2.6); 0.007(0.33) |
| 255 | 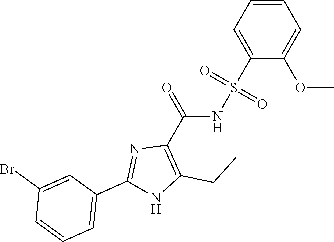 [SMILES: CCc1[nH]c(-c2cccc(Br)c2)nc1C(=O)NS(=O)(=O)c1ccccc1OC] | Example 255: ¹H-NMR(400.0 MHz, d₆-DMSO): δ = 13.05(1.37); 12.729(0.37); 8.569(0.39); 8.276(2.96); 8.211(0.79); 8.055 (1.55); 8.035(1.68); 7.978(0.54); 7.958(0.47); 7.91(1.72); 7.906(1.88); 7.891 (1.88); 7.887(1.93); 7.691(0.72); 7.687(0.74); 7.67(1.49); 7.651(0.9); 7.648 (0.9); 7.628(1.23); 7.607(1.57); 7.571(0.37); 7.553(0.42); 7.506(0.73); 7.487 (1.69); 7.468(2.77); 7.456(0.61); 7.448(1.3); 7.436(1.14); 7.417(0.77); 7.325 (0.64); 7.305(0.65); 7.285(0.72); 7.244(2.15); 7.223(1.97); 7.174(1.17); 7.155(2.12); 7.136(1.04); 4.423(0.82); 4.407(0.84); 3.899(1.25); 3.854(16); 3.326(7.3); 3.017(0.34); 2.998(0.83); 2.979(0.86); 2.961(0.38); 2.891(0.61); 2.857(0.91); 2.838(2.78); 2.82(2.87); 2.801(0.98); 2.732(0.61); 2.675(0.68); 2.671(0.9); 2.666(0.68); 2.524(2.1); 2.51(49.71); 2.506(104.43); 2.502 (146.25); 2.497(110.69); 2.493(54.3); 2.333(0.62); 2.329(0.88); 2.324(0.63); 1.231(0.96); 1.212(2.06); 1.193(0.94); 1.151(3.41); 1.132(7.43); 1.113(3.42); 0.146(0.48); 0.008(3.67); 0(106.69); −0.008(3.7); −0.15(0.48) |
| 257 | 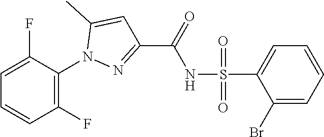 [SMILES: Cc1cc(C(=O)NS(=O)(=O)c2ccccc2Br)nn1-c1c(F)cccc1F] | Example 257: ¹H-NMR(400.0 MHz, d₆-DMSO): δ = 8.202(2.12); 8.198(2.26); 8.182(2.32); 8.178(2.37); 7.862(2.34); 7.843(2.69); 7.783(0.42); 7.766(0.89); 7.761(0.89); 7.745(1.73); 7.729(0.94); 7.724(1.08); 7.708(0.5); 7.68(1.04); 7.662(2.39); 7.645(1.7); 7.627(1.51); 7.623(1.64); 7.608(1.9); 7.604(1.95); 7.589(0.75); 7.585(0.71); 7.476(2.84); 7.454(4.95); 7.434(2.3); 6.928(4.94); 3.338(14.03); 2.67(0.8); 2.506(96.33); 2.501 (130.54); 2.497(102.69); 2.328(0.76); 2.324(0.66); 2.163(16); 2.073(2.46); 0(53.79) |
| 258 | 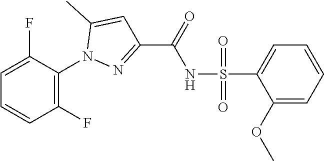 [SMILES: COc1ccccc1S(=O)(=O)NC(=O)c1cc(C)n(-c2c(F)cccc2F)n1] | Example 258: ¹H-NMR(400.0 MHz, d₆-DMSO): δ = 12.126(1.17); 7.908(1.87); 7.904(1.74); 7.888(2.04); 7.884(1.77); 7.764 (0.74); 7.759(0.68); 7.742(1.35); 7.726(0.76); 7.721(0.82); 7.705(0.37); 7.688(0.85); 7.684(0.74); 7.667(1.6); 7.648(1.02); 7.475(2.1); 7.454(3.76); 7.433(1.88); 7.24(2.21); 7.219(2.05); 7.163(1.24); 7.144(2.18); 7.125(1.14); 6.908(3.68); 3.847(16); 3.319(47.29); 2.67(0.55); 2.505(73.9); 2.501 (92.52); 2.497(69.15); 2.328(0.55); 2.324(0.42); 2.16(11.9); 2.073(2.13); 0(39.46) |
| 259 | 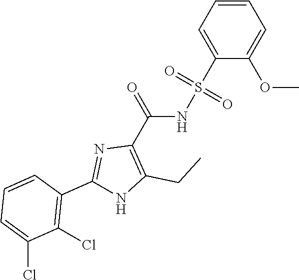 [SMILES: CCc1[nH]c(-c2cccc(Cl)c2Cl)nc1C(=O)NS(=O)(=O)c1ccccc1OC] | Example 259: ¹H-NMR(400.0 MHz, d₆-DMSO): δ = 13.009(1.16); 7.909(1.43); 7.906(1.44); 7.889(1.57); 7.886(1.5); 7.804 (1.44); 7.8(1.53); 7.783(1.7); 7.78(1.81); 7.77(1.6); 7.75(1.91); 7.7(0.66); 7.679(1.37); 7.66(0.81); 7.657(0.75); 7.537(1.52); 7.517(2.43); 7.498(1.1); 7.254(1.89); 7.233(1.76); 7.179(1.04); 7.16(1.89); 7.141(0.95); 4.33(0.34); 4.316(0.35); 3.847(12.24); 3.341(0.62); 3.247(0.34); 2.868(0.78); 2.85 (2.39); 2.831(2.46); 2.812(0.84); 2.671(0.39); 2.506(47.34); 2.502(61.18); 2.498(46.66); 2.328(0.35); 2.074(16); 1.213(0.53); 1.144(2.87); 1.125 (6.09); 1.107(2.85); 0(1.89) |
| 260 | 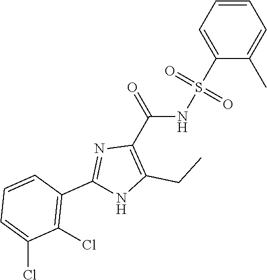 [SMILES: CCc1[nH]c(-c2cccc(Cl)c2Cl)nc1C(=O)NS(=O)(=O)c1ccccc1C] | Example 260: ¹H-NMR(400.0 MHz, d₆-DMSO): δ = 12.988(1.1); 8.037(2.18); 8.02(2.27); 8.018(2.28); 7.797(2.1); 7.793(2.68); 7.777(2.71); 7.773(5.04); 7.769(2.21); 7.753(2.68); 7.749(2); 7.583(0.89); 7.567(2.09); 7.564(2.12); 7.548(1.53); 7.546(1.55); 7.526(2.85); 7.506 (4.43); 7.486(2.05); 7.457(1.32); 7.438(2.04); 7.419(0.95); 7.402(2.27); 7.383(2.01); 7.363(0.49); 4.331(0.5); 4.315(0.51); 2.863(0.99); 2.844(2.96); 2.825(3.06); 2.806(1.06); 2.676(0.36); 2.671(0.45); 2.666(0.34); 2.621(16); 2.587(0.34); 2.524(1.23); 2.511(23.63); 2.506(47.82); 2.502(65.25); 2.497 (48.97); 2.493(24.1); 2.329(0.39); 2.074(1.29); 1.232(0.34); 1.213(0.71); 1.194(0.34); 1.14(4.82); 1.121(10.6); 1.102(4.7); 0(2.69) |
| 261 | 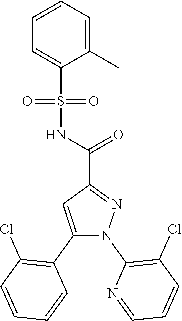 [SMILES: Cc1ccccc1S(=O)(=O)NC(=O)c1cc(-c2ccccc2Cl)n(-c2ncccc2Cl)n1] | Example 261: ¹H-NMR(400.0 MHz, d₆-DMSO): δ = 12.854(0.37); 8.446(2.21); 8.442(2.49); 8.434(2.42); 8.43(2.56); 8.219(2.23); 8.215(2.36); 8.199(2.5); 8.195(2.45); 8.077(2); 8.06(2.03); 8.057(2.09); 7.618(2.64); 7.606(3.93); 7.598(2.57); 7.586(3.34); 7.51(2.01); 7.49(3.63); 7.467(2); 7.448(1.04); 7.436(2.19); 7.419(2.63); 7.416(2.67); 7.401(1.87); 7.397(1.99); 7.381(1.17); 7.376(1.21); 7.323(1.11); 7.32(1.18); 7.304 (2.19); 7.301(2.32); 7.285(1.27); 7.282(1.3); 7.238(8.81); 7.234(3.29); 7.218 (1.68); 7.214(1.54); 3.338(7.93); 2.675(0.44); 2.671(0.61); 2.666(0.51); 2.646(14.14); 2.524(1.58); 2.51(31.81); 2.506(65.97); 2.502(92.05); 2.497 (70.39); 2.493(35.43); 2.333(0.39); 2.328(0.55); 2.324(0.41); 2.074(16); 0(0.37) |
| 262 | 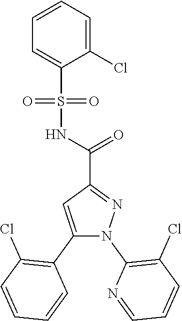 [SMILES: O=C(NS(=O)(=O)c1ccccc1Cl)c1cc(-c2ccccc2Cl)n(-c2ncccc2Cl)n1] | Example 262: ¹H-NMR(400.0 MHz, d₆-DMSO): δ = 8.445(4.84); 8.442(5.41); 8.434(5.25); 8.43(5.43); 8.315(0.49); 8.217(4.83); 8.212(6.56); 8.207(4.71); 8.196(5.48); 8.192(7.65); 8.188(4.93); 7.755(0.91); 7.752(0.96); 7.735(3.07); 7.732(2.91); 7.718(3.83); 7.714(4.11); 7.708(5.34); 7.704(6.66); 7.688(2.37); 7.658(2.64); 7.654(2.42); 7.637(3.69); 7.617(6.59); 7.605(4.94); 7.596(4.7); 7.585(4.54); 7.516(4.31); 7.498(5.83); 7.496(6.08); 7.448(0.38); 7.439(0.44); 7.423(2.36); 7.419(2.58); 7.404(3.84); 7.4(4.09); 7.385(2.42); 7.38(2.51); 7.327(2.38); 7.324(2.55); 7.305(16); 7.289(2.82); 7.286(2.9); 7.243(5.22); 7.239(5.42); 7.224(3.45); 7.22(3.18); 3.368(3.38); 3.321(1.61); 2.676(0.84); 2.671(1.16); 2.666(0.9); 2.524(2.78); 2.51(68.54); 2.506(145.07); 2.502(203.83); 2.497(155.51); 2.493(77.37); 2.333(0.89); 2.329(1.2); 2.324(0.94); 2.074(10.49); 0(1.17) |
| 263 | 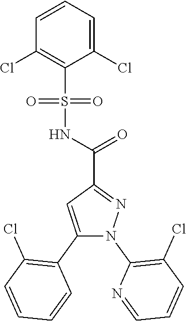 [SMILES: O=C(NS(=O)(=O)c1c(Cl)cccc1Cl)c1cc(-c2ccccc2Cl)n(-c2ncccc2Cl)n1] | Example 263: ¹H-NMR(400.0 MHz, d₆-DMSO): δ = 8.446(5.64); 8.437(5.75); 8.218(4.89); 8.198(5.24); 7.819(0.34); 7.703 (7.04); 7.685(15.04); 7.646(5.62); 7.629(4.67); 7.62(5.71); 7.607(6.25); 7.599(4.68); 7.587(3.88); 7.566(0.79); 7.544(0.94); 7.519(4.69); 7.499 (6.58); 7.424(2.99); 7.405(4.81); 7.386(2.78); 7.326(3.32); 7.306(16); 7.29 (3.77); 7.248(5.85); 7.229(3.69); 5.755(4.16); 3.742(0.33); 3.696(0.4); 3.674(0.47); 3.525(0.7); 3.507(0.73); 3.486(0.69); 3.413(0.59); 3.383(0.52); 2.671(1.22); 2.502(175.01); 2.329(1.14); 1.235(0.79); 0.146(0.52); 0(93.18); −0.15(0.55) |
| 264 | 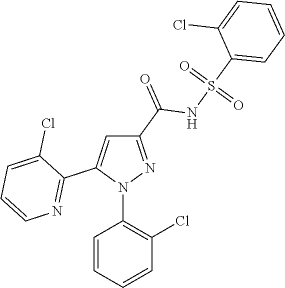 [SMILES: O=C(NS(=O)(=O)c1ccccc1Cl)c1cc(-c2ncccc2Cl)n(-c2ccccc2Cl)n1] | Example 264: ¹H-NMR(400.0 MHz, d₆-DMSO): δ = 8.655(0.32); 8.296(1.83); 8.292(2.08); 8.284(1.96); 8.281(2.08); 8.216(1.53); 8.212(1.7); 8.196(1.69); 8.193(1.79); 8.084(2.03); 8.081(2.21); 8.064(2.26); 8.06(2.3); 7.76(0.39); 7.755(0.4); 7.739(1.23); 7.736(1.19); 7.722(1.56); 7.718(1.66); 7.711(2.09); 7.707(2.76); 7.691(1.04); 7.687(0.7); 7.662(1.41); 7.658(1.45); 7.642(1.49); 7.638(1.27); 7.625(1.81); 7.621(1.08); 7.618(1.06); 7.61(1.54); 7.601(1.66); 7.586(5.58); 7.535(0.38); 7.527(0.68); 7.516(1.38); 7.51(1.57); 7.503(4.04); 7.492(4.7); 7.486(2.35); 7.482(1.81); 7.476(2.04); 7.468(1.96); 7.459(0.67); 7.454(0.35); 7.411(1.89); 7.399(1.8); 7.39(1.78); 7.379(1.78); 5.755(16); 2.671(0.43); 2.666(0.32); 2.524(1.03); 2.511(24.85); 2.506(53.17); 2.502(75.3); 2.498(57.58); 2.493(28.57); 2.334(0.33); 2.329 (0.45); 2.324(0.33); 0.008(2.08); 0(61.16); −0.008(2.35) |
| 265 | 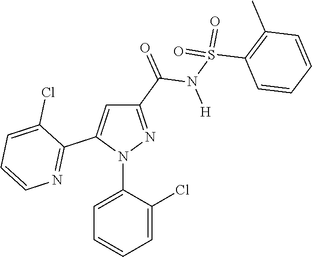 [SMILES: Cc1ccccc1S(=O)(=O)NC(=O)c1cc(-c2ncccc2Cl)n(-c2ccccc2Cl)n1] | Example 265: ¹H-NMR(400.0 MHz, d₆-DMSO): δ = 12.857(0.5); 8.294(2.6); 8.291(2.76); 8.283(2.79); 8.279(2.7); 8.078(3.9); 8.075(2.89); 8.058(3.81); 8.054(2.89); 7.627(2.17); 7.621(1.85); 7.616 (1.64); 7.613(2.38); 7.604(4.29); 7.596(0.7); 7.588(1.52); 7.585(1.45); 7.529 (0.88); 7.518(1.26); 7.511(1.96); 7.504(5.57); 7.498(11.11); 7.495(8.13); 7.488(4.47); 7.483(2.39); 7.478(2.95); 7.47(4.92); 7.461(0.82); 7.452(1.18); 7.436(2.48); 7.418(2.08); 7.408(2.75); 7.396(2.52); 7.387(2.51); 7.375(2.42); 5.755(1.72); 3.357(1.8); 2.675(0.4); 2.671(0.54); 2.666(0.47); 2.647(16); 2.615(1.01); 2.524(1.31); 2.51(29.04); 2.506(57.63); 2.502(77.75); 2.498 (57.85); 2.494(28.21); 2.333(0.34); 2.329(0.45); 2.324(0.33); 0.008(2.48); 0(53.48); −0.008(2.22) |
| 266 | 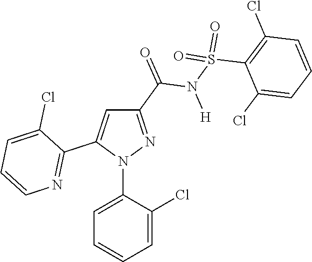 [SMILES: O=C(NS(=O)(=O)c1c(Cl)cccc1Cl)c1cc(-c2ncccc2Cl)n(-c2ccccc2Cl)n1] | Example 266: ¹H-NMR(400.0 MHz, d₆-DMSO): δ = 8.299(4.85); 8.296(5.54); 8.288(5.24); 8.285(5.47); 8.086(4.74); 8.083(5.3); 8.065(5.12); 8.062(5.48); 7.708(5.89); 7.705(7.28); 7.687(16); 7.648(5.93); 7.626(5.95); 7.614(4.6); 7.608(4.28); 7.604(5.11); 7.578(12.17); 7.532(1.45); 7.52(3.1); 7.513(3.97); 7.507(8.37); 7.501(5.34); 7.494(10.28); 7.488(6.86); 7.481(5.13); 7.477(5.06); 7.47(4.79); 7.46(1.09); 7.413(4.29); 7.401(4.21); 7.392(4.15); 7.38(3.94); 2.671(0.65); 2.502(107.31); 2.498(86.93); 2.329 (0.69); 2.075(5.61); 0(60.18) |
| 267 | 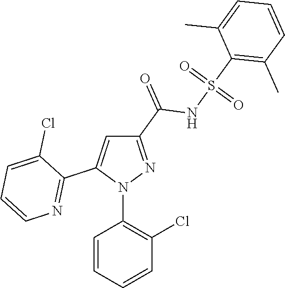 [SMILES: Cc1cccc(C)c1S(=O)(=O)NC(=O)c1cc(-c2ncccc2Cl)n(-c2ccccc2Cl)n1] | Example 267: ¹H-NMR(400.0 MHz, d₆-DMSO): δ = 12.902(0.38); 8.295(1.2); 8.292(1.38); 8.284(1.31); 8.28(1.38); 8.078(1.26); 8.075(1.34); 8.057(1.38); 8.054(1.39); 7.621(0.75); 7.615(0.59); 7.608(0.92); 7.598(1.13); 7.529(0.37); 7.518(1.05); 7.512(3.98); 7.504(2.2); 7.498(1.17); 7.492(2.6); 7.486(1.51); 7.48(1.04); 7.479(1.04); 7.475(1.2); 7.468(1.31); 7.447(0.63); 7.428(1.25); 7.407(1.83); 7.396(1.24); 7.387(1.19); 7.375(1.2); 7.27(2.57); 7.251(2.07); 3.339(1.31); 2.711(16); 2.524(0.62); 2.51(14.02); 2.506(29.24); 2.502(39.23); 2.497(29.07); 2.493(14.72); 2.074(5.07); 0.008 (0.98); 0(27.69); −0.008(1.31) |
| 268 | 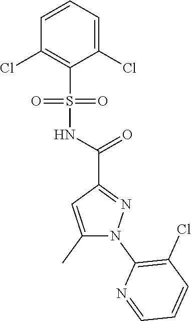 [SMILES: Cc1cc(C(=O)NS(=O)(=O)c2c(Cl)cccc2Cl)nn1-c1ncccc1Cl] | Example 268: ¹H-NMR(400.0 MHz, d₆-DMSO): δ = 8.651(2.27); 8.648(2.43); 8.639(2.43); 8.636(2.41); 8.346(2.23); 8.343(2.3); 8.326(2.43); 8.322(2.42); 7.759(2.12); 7.748(2.08); 7.739(2.04); 7.727(1.97); 7.688(2.56); 7.683(3.2); 7.666(8.23); 7.63(3.05); 7.614(1.88); 7.607(1.43); 7.591(0.95); 6.875(4.87); 2.506(37.31); 2.502(47.65); 2.498(35.34); 2.18 (16); 2.075(0.93); 0(0.69) |
| 269 | 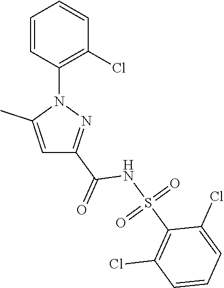 [SMILES: Cc1cc(C(=O)NS(=O)(=O)c2c(Cl)cccc2Cl)nn1-c1ccccc1Cl] | Example 269: ¹H-NMR(400.0 MHz, d₆-DMSO): δ = 7.765(2.06); 7.745(3.07); 7.686(2.79); 7.682(3.25); 7.664(9.66); 7.642(6.07); 7.629(3.52); 7.624(1.99); 7.619(1.11); 7.612(2.07); 7.606(1.6); 7.593(2.21); 7.59(2.61); 7.577(1.24); 7.572(2.28); 7.556(0.77); 7.553(0.66); 6.865(5.02); 2.506(24.93); 2.502(30.7); 2.498(21.96); 2.11(16); 2.086(4.36); 0(8.65) |
| 270 | 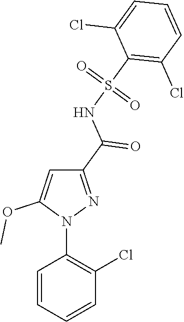 [SMILES: COc1cc(C(=O)NS(=O)(=O)c2c(Cl)cccc2Cl)nn1-c1ccccc1Cl] | Example 270: ¹H-NMR(400.0 MHz, d₆-DMSO): δ = 7.715(1.45); 7.711(1.55); 7.69(3.33); 7.685(2.93); 7.667(6.57); 7.63(3.37); 7.625(1.96); 7.607(3.75); 7.597(2.4); 7.592(2); 7.578(1.29); 7.573(0.93); 7.553(1.49); 7.549(1.53); 7.534(1.64); 7.531(1.64); 7.515(0.65); 6.457 (5.23); 3.892(16); 3.823(0.43); 2.506(29.54); 2.502(38.27); 2.498(28.92); 2.138(0.32); 2.086(4.49); 0(4.24) |
| 271 | 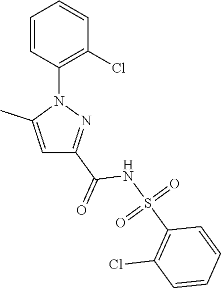 [SMILES: Cc1cc(C(=O)NS(=O)(=O)c2ccccc2Cl)nn1-c1ccccc1Cl] | Example 271: ¹H-NMR(400.0 MHz, d₆-DMSO): δ = 8.174(2); 8.171(2.05); 8.154(2.23); 8.151(2.18); 7.763(2.1); 7.744(2.51); 7.74(2.36); 7.717(1.72); 7.699(2.01); 7.696(1.97); 7.681(3.44); 7.665(1.51); 7.66(1.74); 7.654(2.17); 7.648(1.85); 7.638(4.19); 7.634(4.21); 7.621(3.92); 7.602(1.18); 7.598(1.07); 7.592(2.01); 7.588(1.93); 7.57(2.02); 7.554(0.73); 7.551(0.66); 6.867(4.95); 2.506(25.83); 2.502(32.65); 2.498(24.14); 2.106 (16); 2.086(1.56); 0(0.57) |
| 272 | 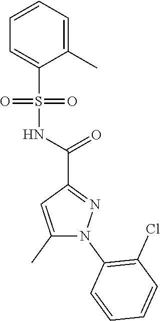 [SMILES: Cc1ccccc1S(=O)(=O)NC(=O)c1cc(C)n(-c2ccccc2Cl)n1] | Example 272: ¹H-NMR(400.0 MHz, d₆-DMSO): δ = 12.559(0.73); 8.042(2.03); 8.04(2.13); 8.023(2.24); 8.02(2.23); 7.764(2.05); 7.744(2.53); 7.74(2.28); 7.661(0.99); 7.656(1.85); 7.654(1.92); 7.649(1.77); 7.639(3.05); 7.634(3.61); 7.63(2.81); 7.622(2.21); 7.617(0.9); 7.603(0.96); 7.6(1.05); 7.593(2.19); 7.589(2.37); 7.584(2.48); 7.581(2.47); 7.576(1.63); 7.571(2.34); 7.566(1.73); 7.563(1.55); 7.555(0.87); 7.552(0.78); 7.466 (1.32); 7.447(2.12); 7.428(1); 7.411(2.33); 7.392(1.93); 6.798(5.08); 6.797 (5.07); 3.339(1.18); 2.605(15.98); 2.51(14.21); 2.506(28.34); 2.502(37.24); 2.497(27.22); 2.1(16); 2.086(1.91); 0.008(2.53); 0(54.94); −0.008(2.77) |
| 273 | 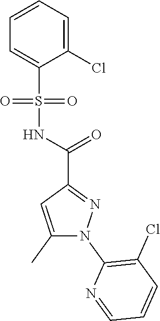 [SMILES: Cc1cc(C(=O)NS(=O)(=O)c2ccccc2Cl)nn1-c1ncccc1Cl] | Example 273: ¹H-NMR(400.0 MHz, d₆-DMSO): δ = 8.648(2.32); 8.645(2.44); 8.637(2.51); 8.633(2.43); 8.344(2.3); 8.341(2.29); 8.324(2.53); 8.32(2.4); 8.175(1.96); 8.172(2.09); 8.155(2.17); 8.152(2.19); 7.757(2.27); 7.745(2.29); 7.736(2.66); 7.724(2.48); 7.719(1.76); 7.702 (1.92); 7.698(1.89); 7.687(2.68); 7.683(3.37); 7.667(1.34); 7.663(1); 7.642 (1.44); 7.638(1.24); 7.622(1.92); 7.604(1.02); 7.6(0.87); 6.878(4.65); 3.364 (0.72); 2.506(43.65); 2.502(56.98); 2.498(41.37); 2.338(0.57); 2.329(0.36); 2.177(16); 2.074(1.04); 0.008(2.47); 0(58.53); −0.008(2.89) |
| 274 | 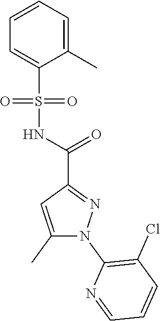 [SMILES: Cc1ccccc1S(=O)(=O)NC(=O)c1cc(C)n(-c2ncccc2Cl)n1] | Example 274: ¹H-NMR(400.0 MHz, d₆-DMSO): δ = 12.606(0.69); 8.648(2.25); 8.645(2.38); 8.637(2.42); 8.633(2.4); 8.345(2.27); 8.341(2.28); 8.324(2.54); 8.321(2.42); 8.043(2.05); 8.025(2.21); 7.757(2.25); 7.745(2.21); 7.737(2.12); 7.725(2.09); 7.602(0.85); 7.587(2.02); 7.584(2.01); 7.568(1.35); 7.565(1.3); 7.468(1.27); 7.449(2.05); 7.431(0.94); 7.413(2.32); 7.394(1.96); 6.811(5.08); 3.335(3.32); 2.607(15.64); 2.506(27.67); 2.502 (36.03); 2.498(26.16); 2.358(0.37); 2.171(16); 2.074(4.06); 0.007(2.15); 0(44.5); −0.008(2.2) |
| 275 | 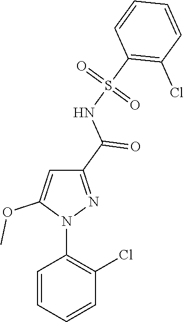 [SMILES: COc1cc(C(=O)NS(=O)(=O)c2ccccc2Cl)nn1-c1ccccc1Cl] | Example 275: ¹H-NMR(400.0 MHz, d₆-DMSO): δ = 8.175(1.58); 8.172(1.62); 8.155(1.76); 8.152(1.71); 7.737(0.44); 7.72(1.41); 7.717(1.48); 7.713(1.71); 7.71(1.56); 7.703(1.8); 7.7(1.8); 7.691(4.22); 7.67 (1.05); 7.642(1.17); 7.638(1.02); 7.622(2.65); 7.618(2.68); 7.61(1.13); 7.6 (3.29); 7.595(2.79); 7.59(1.22); 7.576(1.28); 7.571(0.79); 7.552(1.48); 7.548 (1.45); 7.531(1.43); 7.529(1.54); 7.514(0.56); 7.51(0.51); 6.459(5.52); 3.891 (16); 3.818(0.38); 2.506(29.98); 2.502(38.72); 2.497(28.39); 2.086(1.43); 0.008(0.77); 0(19.74) |
| 276 | 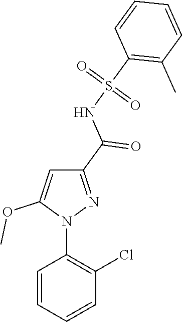 [SMILES: COc1cc(C(=O)NS(=O)(=O)c2ccccc2C)nn1-c1ccccc1Cl] | Example 276: ¹H-NMR(400.0 MHz, d₆-DMSO): δ = 12.546(0.42); 8.045(1.7); 8.025(1.85); 7.712(1.41); 7.709(1.31); 7.692(2.25); 7.622(1.12); 7.617(1.57); 7.609(1.16); 7.599(2.92); 7.594(2.68); 7.588(2.1); 7.575(1.39); 7.57(1.48); 7.552(1.52); 7.548(1.43); 7.532(1.43); 7.53(1.48); 7.514(0.56); 7.511(0.48); 7.469(1.04); 7.45(1.69); 7.43(0.79); 7.416(1.89); 7.397(1.56); 6.395(5.66); 3.883(16); 3.332(1.15); 2.612(12.36); 2.506 (21.77); 2.502(27.37); 2.497(19.58); 2.086(2.75); 0.008(1.31); 0(31.39); −0.008(1.39) |
| 277 | 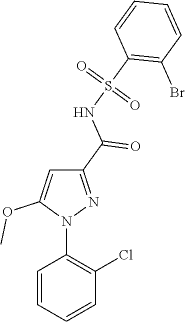 [SMILES: COc1cc(C(=O)NS(=O)(=O)c2ccccc2Br)nn1-c1ccccc1Cl] | Example 277: ¹H-NMR(400.0 MHz, d₆-DMSO): δ = 8.202(1.56); 8.198(1.6); 8.182(1.74); 8.178(1.67); 7.871(1.77); 7.852(2.07); 7.712(1.48); 7.692(2.44); 7.666(1.82); 7.647(1.33); 7.629(1.24); 7.625 (1.47); 7.621(1.46); 7.615(2.06); 7.61(2.44); 7.597(3.25); 7.594(3.27); 7.575 (1.3); 7.57(0.82); 7.551(1.55); 7.548(1.51); 7.529(1.59); 7.513(0.56); 6.473 (5.26); 3.892(16); 2.502(43.53); 2.074(0.37); 0(19.72) |
| 278 | 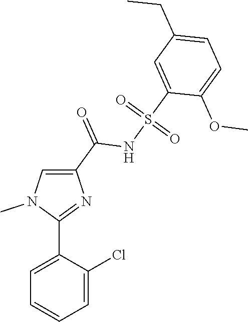 | Example 278: ¹H-NMR(400.0 MHz, d₆-DMSO): δ = 8.148(5.74); 7.71(2.69); 7.705(2.86); 7.671(1.4); 7.653(2.64); 7.618(0.89); 7.613(1.04); 7.6(1.29); 7.595(1.64); 7.576(1.92); 7.571(1.26); 7.557(2.59); 7.552(1.73); 7.529(1.7); 7.519(1.47); 7.512(2.53); 7.498(1.59); 7.492(2.01); 7.16(2.8); 7.139(2.5); 3.811(16); 3.508(14.78); 2.671(1.28); 2.652(3.24); 2.633(3.29); 2.614(1.1); 2.505(34.03); 2.501(44.38); 2.497(32.38); 2.085 (1.41); 1.206(4.21); 1.187(8.73); 1.168(4.03); 0.007(1.67); 0(46.49); −0.008 (2.04) |
| 279 | 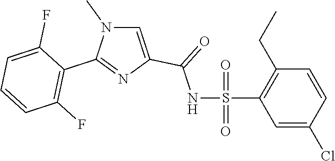 [SMILES: CCc1ccc(Cl)cc1S(=O)(=O)NC(=O)c1cn(C)c(-c2c(F)cccc2F)n1] | Example 279: ¹H-NMR(400.0 MHz, d₆-DMSO): δ = 8.246(1.43); 8.186(5.37); 7.977(4.1); 7.972(4.62); 7.75(0.36); 7.733(0.84); 7.728(0.95); 7.711(2.79); 7.704(2.07); 7.689(2.78); 7.683(2.12); 7.674 (0.53); 7.555(2.16); 7.497(3.23); 7.476(2.68); 7.358(2.92); 7.338(4.76); 7.318(2.56); 3.573(16); 3.058(1.07); 3.039(3.63); 3.02(3.75); 3.002(1.21); 2.735(0.88); 2.716(0.92); 2.507(44.91); 2.502(59.59); 2.498(43.37); 2.329 (0.4); 2.086(0.47); 1.23(1.24); 1.211(2.63); 1.192(1.22); 1.16(4.34); 1.141 (9.55); 1.123(4.34); 0.008(0.56); 0(22.07) |
| 280 | 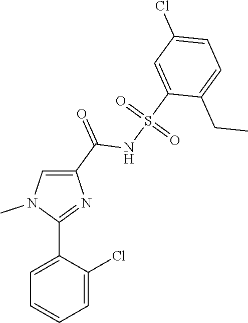 [SMILES: CCc1ccc(Cl)cc1S(=O)(=O)NC(=O)c1cn(C)c(-c2ccccc2Cl)n1] | Example 280: ¹H-NMR(400.0 MHz, d₆-DMSO): δ = 8.173(1.22); 8.118(4.41); 7.969(3.37); 7.964(3.32); 7.678(2.06); 7.672 (1.54); 7.659(2.87); 7.652(1.95); 7.634(0.86); 7.63(1.01); 7.624(0.39); 7.612(1.64); 7.606(1.57); 7.602(1.5); 7.592(1.52); 7.587(2.48); 7.574(0.66); 7.538(2.65); 7.52(1.66); 7.501(0.61); 7.474(2.43); 7.453(2.01); 3.52(16); 3.061(0.85); 3.042(2.81); 3.023(2.88); 3.005(0.92); 2.729(0.75); 2.71(0.76); 2.506(25.52); 2.502(32.85); 2.498(23.68); 2.086(1.13); 1.229(1.03); 1.21 (2.05); 1.191(0.95); 1.162(3.33); 1.144(7.17); 1.125(3.26); 0.008(0.44); 0 (12.14) |
| 281 | 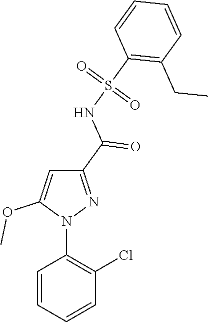 [SMILES: CCc1ccccc1S(=O)(=O)NC(=O)c1cc(OC)n(-c2ccccc2Cl)n1] | Example 281: ¹H-NMR(400.0 MHz, d₆-DMSO): δ = 12.582(0.57); 8.037(1.96); 8.017(2.05); 7.712(1.6); 7.692(2.43); 7.649(0.86); 7.63(1.97); 7.611(3.5); 7.594(4.93); 7.576(1.42); 7.549(1.72); 7.536(1.02); 7.53(1.87); 7.512(0.61); 7.468(2.42); 7.46(1.6); 7.448(2.09); 7.44(2.17); 7.421(0.94); 6.392(5.65); 3.882(16); 3.338(1.14); 3.078(1.04); 3.059(3.23); 3.04(3.3); 3.022(1.1); 2.502(35.7); 2.086(2.78); 1.187(3.79); 1.168(7.86); 1.149(3.69); 0(8.11) |
| 282 | 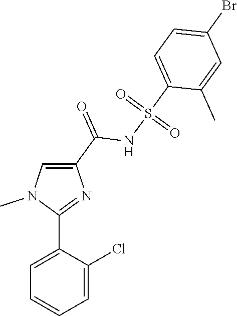 [SMILES: Cc1cc(Br)ccc1S(=O)(=O)NC(=O)c1cn(C)c(-c2ccccc2Cl)n1] | Example 282: ¹H-NMR(400.0 MHz, d₆-DMSO): δ = 8.172(0.63); 8.11(5); 8.056(0.39); 8.036(0.42); 7.935(2.48); 7.927(0.53); 7.92(0.51); 7.912(2.94); 7.67(2.22); 7.66(4.34); 7.654(5.26); 7.645(2.06); 7.622(1.15); 7.618(1.32); 7.605(1.54); 7.6(2); 7.589(1.5); 7.585(1.97); 7.58 (1.58); 7.57(2.84); 7.53(1.77); 7.526(1.7); 7.51(1.97); 7.493(0.76); 7.49 (0.69); 5.755(0.92); 3.518(2.82); 3.51(16); 2.591(13.16); 2.524(0.92); 2.51 (23.04); 2.506(45.66); 2.502(58.99); 2.497(42.12); 2.378(1.63); 2.329(0.35); 0.008(2.88); 0(69.36); −0.008(2.96) |
| 283 | 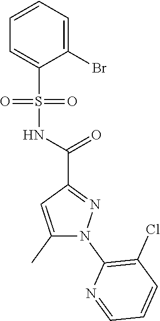 [SMILES: Cc1cc(C(=O)NS(=O)(=O)c2ccccc2Br)nn1-c1ncccc1Cl] | Example 283: ¹H-NMR(400.0 MHz, d₆-DMSO): δ = 8.647(1.4); 8.643(1.57); 8.635(1.52); 8.632(1.55); 8.342(1.39); 8.338(1.47); 8.322(1.56); 8.318(1.55); 8.204(1.2); 8.199(1.34); 8.184(1.27); 8.18(1.31); 7.867(1.14); 7.865(1.22); 7.848(1.4); 7.845(1.44); 7.755(1.4); 7.743(1.36); 7.734(1.34); 7.723(1.32); 7.685(0.5); 7.682(0.6); 7.666(1.26); 7.664(1.32); 7.648(0.96); 7.644(1); 7.628(0.91); 7.624(1.07); 7.609(1.06); 7.605(1.1); 7.59(0.45); 7.586(0.41); 6.894(2.6); 2.506(31.65); 2.502(41.99); 2.497 (31.19); 2.335(0.73); 2.178(8.97); 2.086(16); 0.008(0.89); 0(21.38); −0.008 (1.14) |
| 284 | 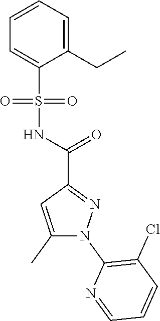 [SMILES: CCc1ccccc1S(=O)(=O)NC(=O)c1cc(C)n(-c2ncccc2Cl)n1] | Example 284: ¹H-NMR(400.0 MHz, d₆-DMSO): δ = 12.644(0.64); 8.646(1.63); 8.644(1.67); 8.635(1.74); 8.632(1.64); 8.344 (1.57); 8.34(1.55); 8.323(1.7); 8.32(1.61); 8.037(1.57); 8.017(1.65); 7.756 (1.41); 7.744(1.41); 7.736(1.37); 7.724(1.3); 7.649(0.67); 7.629(1.46); 7.612(0.94); 7.464(2); 7.444(1.82); 7.439(1.88); 7.42(0.75); 6.808(3.12); 3.33(2.64); 3.073(0.88); 3.055(2.7); 3.036(2.75); 3.018(0.92); 2.67(0.33); 2.502(58.38); 2.328(0.4); 2.168(10.96); 2.086(16); 1.172(3.17); 1.153 (6.51); 1.134(3.07); 0(14.66) |
| 285 | 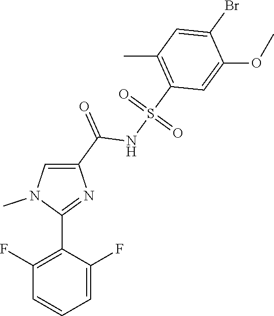 | Example 285: ¹H-NMR(400.0 MHz, d₆-DMSO): δ = 8.187(5.79); 7.747(0.33); 7.73(0.78); 7.726(0.71); 7.709(1.37); 7.692(0.79); 7.688(0.9); 7.674(4.83); 7.632(5.42); 7.357(2.4); 7.337(3.66); 7.316(2.05); 3.907(16); 3.57(12.35); 2.518(15.15); 2.511(18.37); 2.507(33.9); 2.502 (43.57); 2.498(31.35); 2.075(1.04); 0.008(1.38); 0(32.42); −0.008(1.51) |
| 286 | 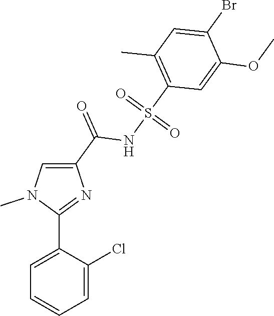 | Example 286: ¹H-NMR(400.0 MHz, d₆-DMSO): δ = 8.11(4.72); 7.672(1.38); 7.655(6.77); 7.631(5.58); 7.624(1.41); 7.619(1.36); 7.606(1.44); 7.601(1.91); 7.593(1.43); 7.589(1.49); 7.582(1.48); 7.574 (2.63); 7.531(1.54); 7.528(1.55); 7.512(1.8); 7.494(0.7); 7.491(0.68); 3.902 (16); 3.868(0.49); 3.512(15.11); 2.516(15.85); 2.506(37.54); 2.502(49.16); 2.498(36.73); 2.329(0.34); 0.008(1.14); 0(28.85) |
| 287 | 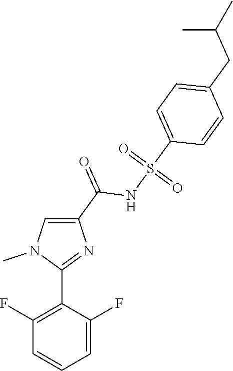 [SMILES: CC(C)Cc1ccc(S(=O)(=O)NC(=O)c2cn(C)c(-c3c(F)cccc3F)n2)cc1] | Example 287: ¹H-NMR(400.0 MHz, d₆-DMSO): δ = 8.161(5.89); 7.912(4.25); 7.892(4.72); 7.724(0.77); 7.719(0.74); 7.702 (1.42); 7.686(0.81); 7.681(0.89); 7.665(0.36); 7.417(4.12); 7.396(3.87); 7.351(2.39); 7.331(3.72); 7.311(2.06); 3.555(13); 2.55(3.84); 2.532(4.09); 2.507(23.51); 2.502(31.25); 2.498(23.29); 2.075(3.75); 1.91(0.42); 1.893 (0.83); 1.877(1.08); 1.86(0.88); 1.843(0.46); 0.87(16); 0.854(15.58); 0.008 (0.93); 0(22.55); −0.008(1.24) |
| 288 | 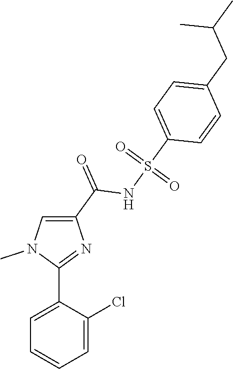 [SMILES: CC(C)Cc1ccc(S(=O)(=O)NC(=O)c2cn(C)c(-c3ccccc3Cl)n2)cc1] | Example 288: ¹H-NMR(400.0 MHz, d₆-DMSO): δ = 8.087(4.92); 8.074(1.24); 8.051(0.47); 8.03(0.46); 7.909(4.03); 7.889(4.58); 7.662(1.86); 7.642(3.45); 7.609(1.64); 7.59(2.56); 7.57(1.77); 7.566(1.82); 7.56(1.81); 7.541(3.15); 7.52(2.36); 7.501(2.37); 7.483(0.84); 7.437(0.38); 7.412(4.44); 7.392(4.31); 3.494(15.45); 2.916(0.8); 2.898(0.82); 2.67(0.45); 2.549(4.44); 2.531(5.51); 2.502(75.77); 2.326(0.49); 1.91(0.51); 1.894 (0.98); 1.876(1.21); 1.86(1.01); 1.843(0.57); 0.871(14.77); 0.855(16); 0 (26.89) |
| 289 | 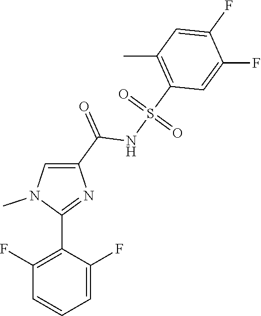 [SMILES: Cc1cc(F)c(F)cc1S(=O)(=O)NC(=O)c1cn(C)c(-c2c(F)cccc2F)n1] | Example 289: ¹H-NMR(400.0 MHz, d₆-DMSO): δ = 8.194(6.2); 8.011(1.34); 7.99(1.53); 7.985(1.62); 7.964(1.31); 7.751(0.38); 7.73(0.95); 7.713(1.66); 7.693(1.07); 7.675(0.43); 7.581(1.16); 7.562(1.28); 7.553(1.31); 7.533(1.14); 7.36(2.82); 7.339(4.68); 7.319(2.48); 3.573(16); 2.574(14.28); 2.502(59.01); 2.499(47.63); 2.329(0.36); 2.075(3.1); 0(39.67) |
| 290 | 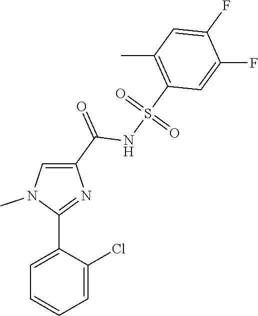 [SMILES: Cc1cc(F)c(F)cc1S(=O)(=O)NC(=O)c1cn(C)c(-c2ccccc2Cl)n1] | Example 290: ¹H-NMR(400.0 MHz, d₆-DMSO): δ = 8.126(5.69); 7.987(1.24); 7.966(1.46); 7.961(1.52); 7.941(1.19); 7.684 (1.51); 7.664(3.23); 7.634(1.35); 7.615(3.38); 7.595(3.8); 7.551(1.2); 7.54 (2.12); 7.532(1.56); 7.522(3.34); 7.503(1.88); 3.522(16); 2.571(13.48); 2.503(44.88); 0(27.12) |
| 291 | 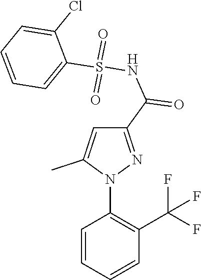 [SMILES: Cc1cc(C(=O)NS(=O)(=O)c2ccccc2Cl)nn1-c1ccccc1C(F)(F)F] | Example 291: ¹H-NMR(400.0 MHz, d₆-DMSO): δ = 8.169(2.18); 8.149(2.34); 8.022(1.85); 8.002(2.32); 7.931(0.72); 7.913 (1.93); 7.895(1.51); 7.868(1.59); 7.849(1.98); 7.83(0.74); 7.736(0.57); 7.714(3.6); 7.696(3.28); 7.689(3.48); 7.685(3.73); 7.669(1.25); 7.64(1.33); 7.638(1.31); 7.62(2.02); 7.604(0.86); 7.6(0.86); 6.862(5.16); 3.339(6.3); 2.503(33.47); 2.08(16); 0(2.51) |
| 292 | 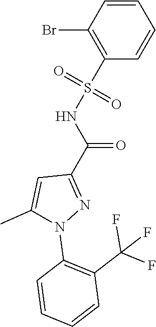 [SMILES: Cc1cc(C(=O)NS(=O)(=O)c2ccccc2Br)nn1-c1ccccc1C(F)(F)F] | Example 292: ¹H-NMR(400.0 MHz, d₆-DMSO): δ = 8.198(1.92); 8.194(2.13); 8.179(2.09); 8.175(2.2); 8.018(1.87); 7.999(2.32); 7.93(0.75); 7.912(1.96); 7.894(1.56); 7.866(3.61); 7.847(4.33); 7.829(0.77); 7.707(2.38); 7.688(2.11); 7.662(2.18); 7.645(1.55); 7.642(1.52); 7.626 (1.37); 7.622(1.52); 7.607(1.75); 7.603(1.83); 7.588(0.72); 7.584(0.66); 6.873(4.28); 3.339(83.78); 2.672(0.46); 2.507(59.97); 2.503(79.18); 2.499 (60.87); 2.33(0.45); 2.08(16); 0.008(2.9); 0(65.62); −0.15(0.33) |
| 293 | 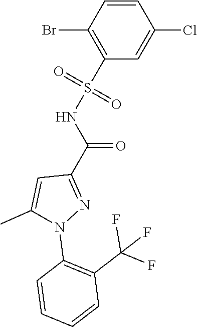 [SMILES: Cc1cc(C(=O)NS(=O)(=O)c2cc(Cl)ccc2Br)nn1-c1ccccc1C(F)(F)F] | Example 293: ¹H-NMR(400.0 MHz, d₆-DMSO): δ = 8.12(4.17); 8.113(4.39); 8.023(1.87); 8.003(2.34); 7.934(0.74); 7.914(2.09); 7.908(3.76); 7.898(1.77); 7.886(4.48); 7.871(1.76); 7.852(2.09); 7.833 (0.74); 7.732(2.37); 7.725(2.62); 7.718(2.73); 7.71(2.26); 7.703(2.7); 6.879 (5.22); 2.508(18.3); 2.504(23.94); 2.5(18.48); 2.083(16); 1.235(0.43); 0 (2.35) |
| 294 | 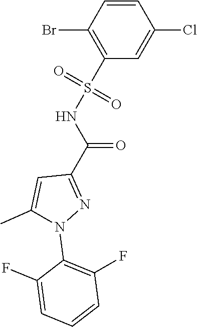 [SMILES: Cc1cc(C(=O)NS(=O)(=O)c2cc(Cl)ccc2Br)nn1-c1c(F)cccc1F] | Example 294: ¹H-NMR(400.0 MHz, d₆-DMSO): δ = 8.259(6.01); 8.103(4.18); 8.097(4.17); 7.865(3.15); 7.844(3.93); 7.767 (0.42); 7.75(1.07); 7.729(1.78); 7.71(1.17); 7.687(2.46); 7.681(2.23); 7.666(1.83); 7.66(1.77); 7.374(2.89); 7.354(4.94); 7.333(2.45); 3.6(16); 2.502(61.25); 2.329(0.41); 2.086(4.57); 0(5.77) |
| 295 | 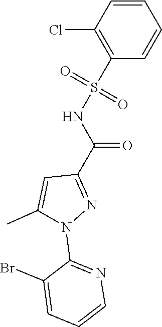 [SMILES: Cc1cc(C(=O)NS(=O)(=O)c2ccccc2Cl)nn1-c1ncccc1Br] | Example 295: ¹H-NMR(400.0 MHz, d₆-DMSO): δ = 8.676(2.29); 8.673(2.5); 8.664(2.49); 8.661(2.52); 8.464(2.25); 8.46(2.33); 8.443(2.45); 8.44(2.42); 8.173(2.17); 8.153(2.3); 7.737(0.54); 7.717(1.68); 7.699(1.95); 7.683(3.35); 7.663(3.16); 7.651(2.23); 7.642(3.27); 7.631 (2.37); 7.621(2.13); 7.602(0.93); 6.874(4.34); 5.756(2.48); 3.344(2.68); 2.671(0.32); 2.506(43.04); 2.502(55.73); 2.498(42.52); 2.329(0.33); 2.28 (0.5); 2.157(16); 0.008(1.87); 0(40.12) |
| 296 | 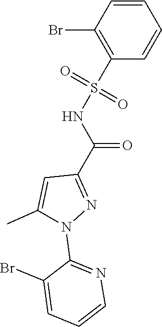 [SMILES: Cc1cc(C(=O)NS(=O)(=O)c2ccccc2Br)nn1-c1ncccc1Br] | Example 296: ¹H-NMR(400.0 MHz, d₆-DMSO): δ = 8.675(2.37); 8.672(2.67); 8.664(2.54); 8.66(2.63); 8.463(2.42); 8.459(2.56); 8.443(2.64); 8.439(2.6); 8.204(2.04); 8.2(2.25); 8.185(2.23); 8.181(2.32); 7.868(2.06); 7.865(2.25); 7.849(2.51); 7.846(2.64); 7.687(0.87); 7.684 (1.04); 7.668(2.27); 7.665(2.49); 7.662(3); 7.65(3.88); 7.641(2.59); 7.63 (3.77); 7.625(1.96); 7.61(1.94); 7.606(2.04); 7.592(0.81); 7.587(0.74); 6.89 (5.12); 5.756(10.22); 3.362(1.74); 2.506(39.94); 2.502(53.18); 2.497(39.8); 2.158(16); 2.086(0.94); 0.008(1.65); 0(40.47); −0.008(1.89) |
| 297 | 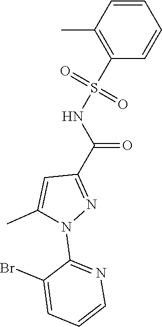 [SMILES: Cc1ccccc1S(=O)(=O)NC(=O)c1cc(C)n(-c2ncccc2Br)n1] | Example 297: ¹H-NMR(400.0 MHz, CDCl3): δ = 9.409(2.2); 8.628(2.24); 8.625(2.13); 8.617(2.33); 8.614(2.04); 8.298(2.17); 8.278(2.27); 8.208(2.24); 8.205(2.01); 8.188(2.38); 8.184(2.06); 7.547 (0.93); 7.528(2.19); 7.51(1.47); 7.459(1.89); 7.448(2.07); 7.439(3.14); 7.427(2.14); 7.42(2.23); 7.402(0.92); 7.324(2.3); 7.305(2); 7.286(10.34); 6.703(4.89); 5.323(0.88); 3.769(0.34); 2.702(15.42); 2.254(16); 1.876(0.37); 1.651(9.45) |
| 298 | 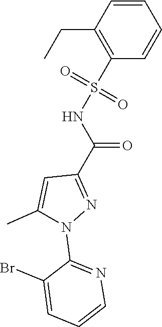 [SMILES: CCc1ccccc1S(=O)(=O)NC(=O)c1cc(C)n(-c2ncccc2Br)n1] | Example 298: ¹H-NMR(601.6 MHz, CDCl3): δ = 9.378(1.34); 8.625(2.6); 8.622(2.62); 8.617(2.71); 8.615(2.65); 8.303(1.87); 8.301(1.95); 8.29(1.97); 8.288(1.99); 8.201(2.7); 8.199(2.79); 8.188(3.04); 8.185(2.88); 7.592(0.89); 7.59(0.92); 7.58(1.96); 7.577(1.97); 7.567(1.23); 7.565(1.19); 7.45(2.93); 7.443(2.87); 7.437(2.72); 7.429(2.79); 7.425(1.04); 7.423(1.18); 7.411(1.78); 7.4(0.89); 7.398(1.05); 7.394(1.96); 7.381(1.7); 7.284(15.53); 6.702(4.61); 6.701(4.57); 3.128(1.22); 3.115(3.89); 3.103 (3.96); 3.09(1.3); 2.249(16); 2.248(15.51); 2.027(0.36); 1.628(0.51); 1.303 (6.29); 1.291(13.69); 1.278(6.61) |
| 299 | 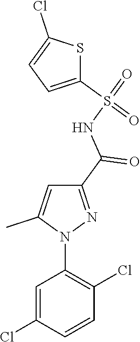 [SMILES: Cc1cc(C(=O)NS(=O)(=O)c2ccc(Cl)s2)nn1-c1cc(Cl)ccc1Cl] | Example 299: ¹H-NMR(400.0 MHz, d₆-DMSO): δ = 7.867(3.96); 7.861(4.35); 7.81(2.62); 7.788(5.67); 7.757(3.45); 7.751(3.1); 7.735(1.58); 7.729(1.58); 7.719(3.84); 7.708(3.99); 7.56(1.01); 7.302(4.52); 7.291(4.25); 6.841(5.01); 3.436(0.32); 3.425(0.32); 3.42(0.33); 2.671(0.43); 2.667(0.35); 2.506(48.77); 2.502(66.89); 2.498(52.01); 2.329(0.39); 2.236 (1.12); 2.197(0.39); 2.143(16); 2.086(3.44); 1.235(1.07); 0.008(2.55); 0 (59.44) |
| 300 | 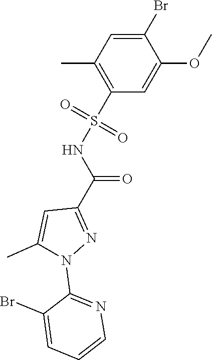 | Example 300: ¹H-NMR(400.0 MHz, CDCl3): δ = 9.323(1.42); 8.538(1.66); 8.534(1.89); 8.527(1.75); 8.523(1.86); 8.116 (1.71); 8.112(1.81); 8.096(1.84); 8.092(1.86); 7.692(4.92); 7.409(4.25); 7.371(1.7); 7.359(1.68); 7.35(1.62); 7.339(1.58); 7.194(6.59); 6.62(4); 3.93 (16); 2.494(13.49); 2.167(12.96); 1.937(2.28); 1.579(0.74) |
| 301 | 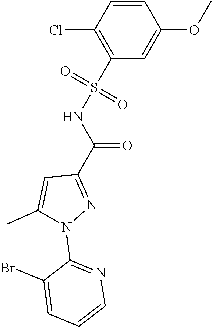 | Example 301: ¹H-NMR(601.6 MHz, CDCl3): δ = 9.605(0.61); 8.62(1.51); 8.617(1.66); 8.612(1.58); 8.609(1.67); 8.201(1.67); 8.198(1.76); 8.188(1.74); 8.185(1.77); 7.875(2.3); 7.87(2.43); 7.443(1.75); 7.436(1.74); 7.43(1.69); 7.422(1.68); 7.386(2.88); 7.372(3.17); 7.284 (11.07); 7.093(1.62); 7.088(1.62); 7.079(1.47); 7.074(1.47); 6.714(2.98); 6.713(3.31); 3.918(16); 2.268(11.21); 1.619(0.74) |
| 302 | 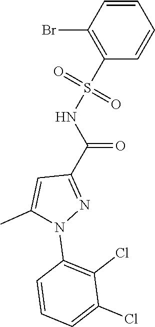 [SMILES: Cc1cc(C(=O)NS(=O)(=O)c2ccccc2Br)nn1-c1cccc(Cl)c1Cl] | Example 302: ¹H-NMR(400.0 MHz, CDCl3): δ = 9.565(1.61); 8.418(1.74); 8.415(1.83); 8.399(1.85); 8.395(1.89); 7.721 (1.98); 7.719(2.02); 7.702(4.17); 7.699(3.99); 7.682(2.24); 7.678(2.22); 7.564(0.91); 7.562(0.95); 7.545(2.1); 7.543(2.08); 7.526(1.39); 7.523(1.29); 7.477(1.34); 7.473(1.41); 7.458(1.85); 7.454(1.89); 7.439(2.39); 7.434 (0.95); 7.419(3.69); 7.399(2.3); 7.352(2.56); 7.348(2.66); 7.332(1.65); 7.328(1.52); 7.262(10); 6.683(4.72); 5.3(7.02); 2.155(16); 1.584(2.86) |
| 303 | 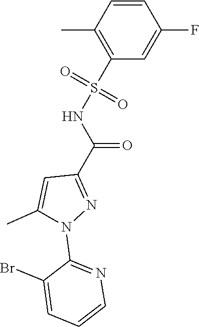 [SMILES: Cc1ccc(F)cc1S(=O)(=O)NC(=O)c1cc(C)n(-c2ncccc2Br)n1] | Example 303: ¹H-NMR(400.0 MHz, d₆-DMSO): δ = 8.678(2.54); 8.675(2.77); 8.666(2.73); 8.663(2.72); 8.468(2.4); 8.464(2.49); 8.447(2.57); 8.444(2.56); 7.785(1.5); 7.779(1.68); 7.763(1.57); 7.757(1.68); 7.666(2.28); 7.654(2.26); 7.646(2.23); 7.634(2.14); 7.516(0.34); 7.51(0.32); 7.495(1.48); 7.489(1.64); 7.48(2.44); 7.476(2.26); 7.467(2.87); 7.445(0.47); 6.809(4.15); 3.333(2.51); 2.571(13.81); 2.506(37.4); 2.502(49.92); 2.498 (38.3); 2.154(16); 2.074(0.59); 0.008(2.12); 0(43.21) |
| 304 | 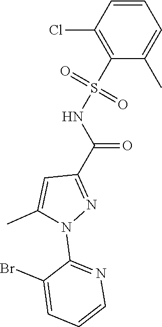 | Example 304: ¹H-NMR(400.0 MHz, d₆-DMSO): δ = 12.786(0.37); 8.674(2.32); 8.67(2.54); 8.663(2.48); 8.659(2.52); 8.461 (2.31); 8.457(2.36); 8.441(2.53); 8.437(2.42); 7.66(2.33); 7.648(2.25); 7.64(2.22); 7.628(2.22); 7.546(0.56); 7.526(2.26); 7.509(5.12); 7.506(4.08); 7.492(0.8); 7.427(1.66); 7.421(1.61); 7.41(1.16); 7.405(1.15); 6.871(4.18); 3.331(5.56); 2.741(16); 2.67(0.33); 2.524(1.11); 2.51(20.07); 2.506(41.26); 2.502(56.94); 2.497(43.08); 2.493(21.42); 2.328(0.34); 2.155(14.71); 2.074 (10.65); 0.008(1.99); 0(51.3); −0.008(2.03) |
| 305 | 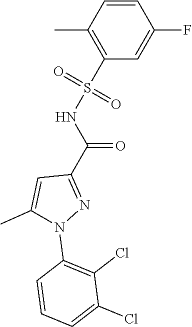 [SMILES: Cc1ccc(F)cc1S(=O)(=O)NC(=O)c1cc(C)n(-c2cccc(Cl)c2Cl)n1] | Example 305: ¹H-NMR(400.0 MHz, CDCl3): δ = 9.337(0.38); 7.996(1.68); 7.989(1.75); 7.974(1.73); 7.968(1.74); 7.706 (2.14); 7.702(2.25); 7.686(2.55); 7.682(2.56); 7.443(1.79); 7.423(3.96); 7.403(2.4); 7.347(2.84); 7.343(2.94); 7.327(1.91); 7.323(1.82); 7.286(0.86); 7.273(1.08); 7.263(5.53); 7.252(1.83); 7.233(1.3); 7.226(1.27); 7.214(1.58); 7.207(1.57); 7.193(0.64); 7.186(0.63); 6.697(4.85); 6.695(4.71); 2.63 (13.16); 2.158(16); 2.008(0.34); 1.618(0.79); 0.071(0.49); 0(4.14) |
| 306 | 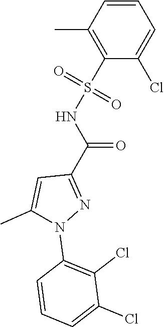 [SMILES: Cc1cccc(Cl)c1S(=O)(=O)NC(=O)c1cc(C)n(-c2cccc(Cl)c2Cl)n1] | Example 306: ¹H-NMR(400.0 MHz, CDCl3): δ = 7.698(1.79); 7.694(1.91); 7.678(2.12); 7.674(2.18); 7.435(1.55); 7.416 (3.52); 7.395(2.19); 7.376(0.49); 7.363(5.99); 7.356(3.15); 7.348(5.55); 7.329(2.11); 7.325(1.67); 7.28(1.64); 7.273(1.23); 7.262(5.68); 6.679(4.3); 6.678(4.25); 2.896(16); 2.155(14.58); 2.007(1.78); 1.59(0.42); 0(4.75) |
| 307 | 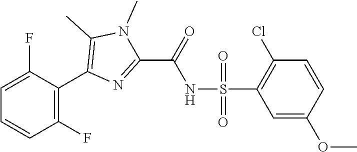 [SMILES: COc1ccc(Cl)c(S(=O)(=O)NC(=O)c2nc(-c3c(F)cccc3F)c(C)n2C)c1] | Example 307: ¹H-NMR(400.0 MHz, d₆-DMSO): δ = 7.66(0.78); 7.642(1.34); 7.622(0.9); 7.605(0.37); 7.586(2.89); 7.579(3.04); 7.435(2.55); 7.413(2.96); 7.323(2.18); 7.303(3.76); 7.283(1.91); 7.126 (1.57); 7.118(1.59); 7.104(1.41); 7.096(1.4); 4.027(13.69); 3.822(16); 2.502 (83.66); 2.328(0.63); 2.186(11.93); 2.074(1.98); 0(43.88) |
| 308 | 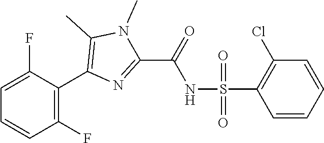 [SMILES: Cc1c(-c2c(F)cccc2F)nc(C(=O)NS(=O)(=O)c2ccccc2Cl)n1C] | Example 308: ¹H-NMR(400.0 MHz, d₆-DMSO): δ = 8.098(2.18); 8.078(2.47); 7.673(0.35); 7.653(0.92); 7.635(1.56); 7.614 (1.03); 7.597(0.43); 7.542(1.67); 7.53(6.58); 7.513(1.88); 7.506(1.2); 7.493 (1.61); 7.486(1.05); 7.479(0.81); 7.472(0.58); 7.317(2.58); 7.297(4.38); 7.277(2.21); 4.01(16); 2.671(0.53); 2.501(98.05); 2.328(0.58); 2.181 (13.93); 2.074(0.35); 0(57.85) |
| 309 | 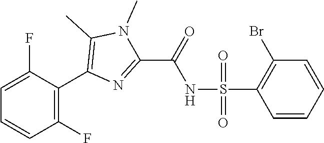 [SMILES: Cc1c(-c2c(F)cccc2F)nc(C(=O)NS(=O)(=O)c2ccccc2Br)n1C] | Example 309: ¹H-NMR(400.0 MHz, d₆-DMSO): δ = 8.127(1.95); 8.123(1.89); 8.108(2.16); 8.104(2); 7.724(2.28); 7.704(2.71); 7.675(0.33); 7.657(0.78); 7.636(1.42); 7.616(0.88); 7.599(0.37); 7.558 (1.08); 7.539(2.34); 7.52(1.52); 7.457(1.26); 7.453(1.21); 7.438(1.86); 7.419(0.84); 7.415(0.75); 7.319(2.47); 7.299(4.18); 7.278(2.14); 4.206 (0.34); 4.029(16); 2.671(0.88); 2.505(126.27); 2.502(157.78); 2.498 (113.15); 2.328(0.95); 2.186(13.17); 2.074(0.9); 0.146(0.51); 0.008(4.99); 0(109.73); −0.008(4.65); −0.149(0.52) |
| 310 | 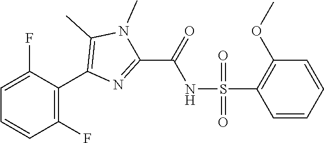 [SMILES: COc1ccccc1S(=O)(=O)NC(=O)c1nc(-c2c(F)cccc2F)c(C)n1C] | Example 310: ¹H-NMR(400.0 MHz, d₆-DMSO): δ = 7.906(0.9); 7.902(0.92); 7.887(0.97); 7.883(0.96); 7.671(0.39); 7.667(0.38); 7.65(0.79); 7.631(0.49); 7.628(0.45); 7.578(0.38); 7.574(0.37); 7.557(0.7); 7.54(0.4); 7.536(0.45); 7.264(1.15); 7.245(1.84); 7.227(1.66); 7.208(1.12); 7.154(0.65); 7.135(1.17); 7.116(0.57); 3.835(16); 2.506(34.46); 2.501 (44.36); 2.497(32.53); 2.14(6.03); 0.008(1.06); 0(32.03) |
| 311 | 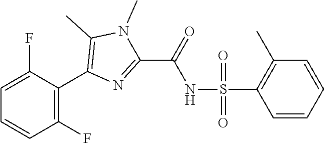 [SMILES: Cc1ccccc1S(=O)(=O)NC(=O)c1nc(-c2c(F)cccc2F)c(C)n1C] | Example 311: ¹H-NMR(400.0 MHz, d₆-DMSO): δ = 8.006(1.86); 7.987(2); 7.929(0.32); 7.601(0.74); 7.597(0.76); 7.58(1.34); 7.559(0.89); 7.542(0.37); 7.514(0.66); 7.495(1.61); 7.477(1.12); 7.418(0.6); 7.399(1.49); 7.38(1.77); 7.361(0.85); 7.343(1.94); 7.325(1.57); 7.275(2.32); 7.256(3.69); 7.235(1.99); 3.883(16); 2.671(0.39); 2.604(15.26); 2.575 (2.39); 2.524(0.9); 2.506(57.93); 2.502(75.17); 2.497(54.32); 2.328(0.44); 2.324(0.34); 2.236(0.45); 2.218(0.87); 2.199(0.49); 2.143(12.52); 2.074 (1.52); 1.376(0.44); 1.358(0.35); 1.151(0.39); 1.132(0.38); 0.808(0.81); 0.79(1.6); 0.771(0.65); 0.008(1.98); 0(57.25); −0.008(2.58) |
| 312 | 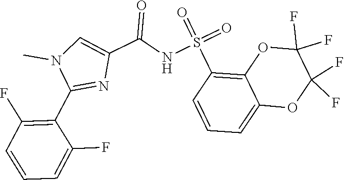 [SMILES: Cn1cc(C(=O)NS(=O)(=O)c2cccc3c2OC(F)(F)C(F)(F)O3)nc1-c1c(F)cccc1F] | Example 312: ¹H-NMR(400.0 MHz, d₆-DMSO): δ = 8.225(3.59); 7.93(2.15); 7.927(2.19); 7.91(2.42); 7.908(2.39); 7.81(1.84); 7.79(2.18); 7.765(0.44); 7.748(0.98); 7.744(0.9); 7.727(1.72); 7.71(1); 7.706(1.09); 7.689(0.46); 7.583(1.93); 7.562(3.22); 7.542(1.53); 7.372 (2.96); 7.351(4.85); 7.331(2.6); 3.932(0.33); 3.873(0.32); 3.773(0.37); 3.64 (0.37); 3.598(16); 2.67(0.7); 2.506(107.11); 2.502(140.54); 2.497(104.23); 2.328(0.88); 2.074(3.07); 0.146(0.5); 0.008(4.8); 0(118.21); −0.008(6.86); −0.15(0.58) |
| 313 | 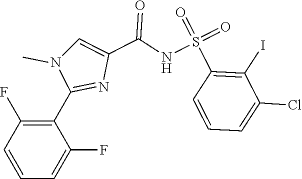 [SMILES: Cn1cc(C(=O)NS(=O)(=O)c2cccc(Cl)c2I)nc1-c1c(F)cccc1F] | Example 313: ¹H-NMR(400.0 MHz, d₆-DMSO): δ = 8.262(3.56); 8.119(2.31); 8.116(2.25); 8.099(2.57); 8.096(2.35); 7.883(2); 7.864(2.37); 7.753(0.42); 7.736(0.98); 7.732(0.94); 7.715(1.73); 7.698 (1.03); 7.694(1.06); 7.677(0.61); 7.668(1.92); 7.648(3.28); 7.628(1.5); 7.363(2.99); 7.343(4.83); 7.322(2.54); 3.587(16); 2.671(0.48); 2.506 (79.32); 2.502(100.86); 2.498(74.03); 2.329(0.65); 2.075(0.68); 0(54.3); −0.008(3.24) |
| 314 | 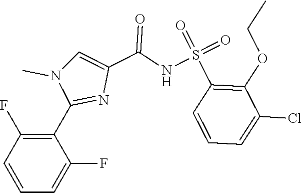 [SMILES: CCOc1c(Cl)cccc1S(=O)(=O)NC(=O)c1cn(C)c(-c2c(F)cccc2F)n1] | Example 314: ¹H-NMR(400.0 MHz, d₆-DMSO): δ = 8.203(4.19); 7.917(2.08); 7.913(2.46); 7.897(2.4); 7.893(2.59); 7.85(1.86); 7.846(1.83); 7.83(2.16); 7.826(1.97); 7.748(0.41); 7.732(0.92); 7.727(0.92); 7.711(1.7); 7.694(0.95); 7.689(1.04); 7.672(0.45); 7.392(1.97); 7.371(4.11); 7.363(3.19); 7.351(2.43); 7.343(4.68); 7.322(2.57); 4.184(1.33); 4.166 (4.39); 4.149(4.44); 4.132(1.38); 3.577(16); 3.54(0.43); 3.421(0.71); 3.397 (0.79); 3.363(0.81); 3.349(0.79); 3.298(0.72); 2.675(0.61); 2.67(0.81); 2.506(106.06); 2.502(142.23); 2.497(104.88); 2.333(0.64); 2.328(0.85); 2.074(2.96); 1.419(4.59); 1.401(10.08); 1.384(4.55); 0.008(2.28); 0(56.33); −0.008(2.73) |
| 315 | 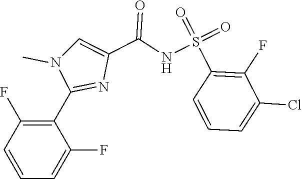 [SMILES: Cn1cc(C(=O)NS(=O)(=O)c2cccc(Cl)c2F)nc1-c1c(F)cccc1F] | Example 315: ¹H-NMR(400.0 MHz, d₆-DMSO): δ = 8.229(5.4); 7.951(3.02); 7.931(4.27); 7.914(3.21); 7.764(0.41); 7.748(0.95); 7.744(0.94); 7.726(1.67); 7.705(1.1); 7.688(0.46); 7.482(1.42); 7.462(2.64); 7.442(1.33); 7.372(2.94); 7.351(4.76); 7.331(2.51); 3.987(0.35); 3.962 (0.34); 3.947(0.34); 3.924(0.36); 3.889(0.36); 3.884(0.38); 3.864(0.36); 3.837(0.38); 3.8(0.38); 3.784(0.37); 3.767(0.41); 3.748(0.36); 3.738(0.35); 3.701(0.35); 3.656(0.33); 3.637(0.39); 3.592(16); 3.541(0.34); 2.671(0.83); 2.506(118.74); 2.502(157.05); 2.497(117.87); 2.328(0.94); 2.324(0.71); 2.074(0.43); 0.146(0.35); 0.008(3.73); 0(83.5); −0.15(0.34) |
| 316 | 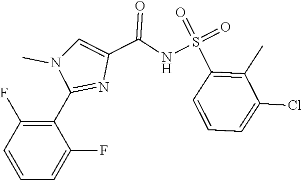 [SMILES: Cc1c(Cl)cccc1S(=O)(=O)NC(=O)c1cn(C)c(-c2c(F)cccc2F)n1] | Example 316: ¹H-NMR(400.0 MHz, d₆-DMSO): δ = 8.178(3.72); 8.046(2.19); 8.026(2.36); 7.786(1.91); 7.766(2.17); 7.749 (0.39); 7.732(0.85); 7.728(0.86); 7.711(1.62); 7.694(0.98); 7.689(0.96); 7.672(0.38); 7.496(1.45); 7.476(2.51); 7.456(1.18); 7.359(2.71); 7.339 (4.32); 7.318(2.3); 3.818(0.32); 3.806(0.34); 3.778(0.36); 3.746(0.43); 3.715(0.45); 3.703(0.47); 3.683(0.48); 3.661(0.54); 3.63(0.63); 3.618(0.67); 3.57(15.39); 3.472(0.84); 3.462(0.85); 3.431(0.85); 3.392(0.87); 3.372 (0.77); 3.336(0.71); 3.28(0.6); 3.189(0.42); 3.182(0.41); 3.119(0.33); 2.671 (0.88); 2.64(16); 2.506(105.92); 2.502(135.14); 2.497(101.39); 2.328(0.82); 2.074(0.42); 0(67.22); −0.149(0.32) |
| 317 | 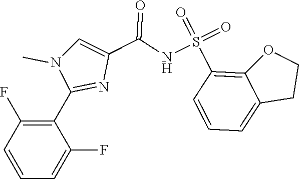 [SMILES: Cn1cc(C(=O)NS(=O)(=O)c2cccc3c2OCC3)nc1-c1c(F)cccc1F] | Example 317: ¹H-NMR(400.0 MHz, d₆-DMSO): δ = 8.216(3.92); 7.741(0.41); 7.724(0.96); 7.72(0.91); 7.703(1.72); 7.686(0.97); 7.682(1.04); 7.665(0.46); 7.607(2.19); 7.587(2.38); 7.533(1.89); 7.516 (2.06); 7.354(2.93); 7.334(4.7); 7.314(2.51); 7.015(1.85); 6.996(2.96); 6.976(1.67); 4.678(2.27); 4.656(4.75); 4.634(2.44); 3.568(16); 3.321(1.14); 3.314(1.12); 3.262(2.49); 3.24(4.17); 3.218(2.15); 2.67(0.46); 2.506(60.8); 2.502(78.31); 2.497(57.32); 2.328(0.46); 2.074(1.83); 0.008(2.06); 0(41.85) |
| 318 | 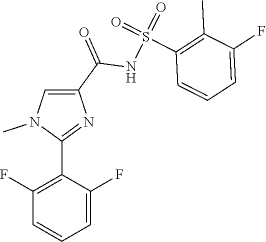 [SMILES: Cc1c(F)cccc1S(=O)(=O)NC(=O)c1cn(C)c(-c2c(F)cccc2F)n1] | Example 318: ¹H-NMR(400.0 MHz, d₆-DMSO): δ = 8.182(4.79); 7.897(1.88); 7.877(2.13); 7.747(0.39); 7.73(1.01); 7.709(1.7); 7.691(1.11); 7.671(0.44); 7.542(0.52); 7.522(1.83); 7.512(1.59); 7.496 (3.27); 7.478(1.25); 7.458(0.34); 7.357(2.82); 7.337(4.71); 7.316(2.4); 3.569(16); 3.39(0.37); 2.67(0.64); 2.502(118.05); 2.328(0.68); 2.074(4.31); 1.236(0.53); 0.146(0.36); 0(82.7); −0.149(0.39) |
| 319 | 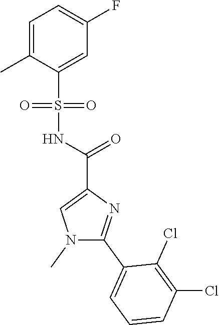 [SMILES: Cc1ccc(F)cc1S(=O)(=O)NC(=O)c1cn(C)c(-c2cccc(Cl)c2Cl)n1] | Example 319: ¹H-NMR(400.0 MHz, d₆-DMSO): δ = 8.135(4.73); 7.881(1.77); 7.876(1.88); 7.862(2.03); 7.857(1.99); 7.765 (1.44); 7.76(1.53); 7.739(1.64); 7.588(0.92); 7.583(1.22); 7.569(3.02); 7.564(2.59); 7.554(2.76); 7.534(3.02); 7.515(1.02); 7.462(1.74); 7.456(3.2); 7.439(3.48); 5.757(1.32); 4.045(0.52); 3.801(0.41); 3.69(0.35); 3.63(0.37); 3.515(16); 3.185(0.54); 2.671(0.41); 2.569(12.87); 2.549(0.52); 2.506 (50.84); 2.502(63.25); 2.498(46.47); 2.328(0.35); 1.236(1.16); 0(6.02) |
| 320 | 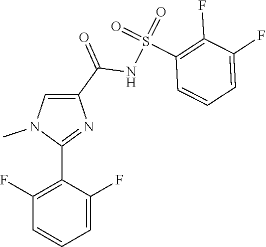 [SMILES: Cn1cc(C(=O)NS(=O)(=O)c2cccc(F)c2F)nc1-c1c(F)cccc1F] | Example 320: ¹H-NMR(400.0 MHz, d₆-DMSO): δ = 8.184(1.83); 8.134(0.75); 7.794(0.43); 7.776(1.64); 7.756(2.16); 7.749 (1.75); 7.74(1.55); 7.735(1.42); 7.718(1.83); 7.702(0.98); 7.697(1.09); 7.68 (0.47); 7.452(0.62); 7.443(0.68); 7.432(1.13); 7.424(1.06); 7.412(0.63); 7.403(0.5); 7.365(3.15); 7.345(5.01); 7.324(2.7); 7.207(0.54); 7.079(0.58); 6.952(0.55); 3.584(16); 2.525(0.4); 2.507(50.53); 2.502(69.14); 2.498 (53.24); 2.333(0.39); 2.329(0.5); 2.325(0.4); 2.075(2.09); 0.008(1.74); 0 (65.48); −0.008(3.85); −0.15(0.36) |
| 321 | 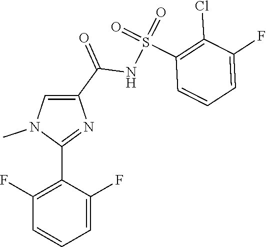 [SMILES: Cn1cc(C(=O)NS(=O)(=O)c2cccc(F)c2Cl)nc1-c1c(F)cccc1F] | Example 321: ¹H-NMR(400.0 MHz, d₆-DMSO): δ = 8.248(6.13); 8.009(2.15); 7.99(2.38); 7.782(0.91); 7.761(2.05); 7.74(2.07); 7.719(1.73); 7.702(0.96); 7.698(1.07); 7.678(1.3); 7.665(1.28); 7.658(1.73); 7.645(1.72); 7.637(0.78); 7.624(0.69); 7.365(3.07); 7.345(4.83); 7.325(2.6); 3.589(16); 2.675(0.38); 2.671(0.52); 2.506(67.99); 2.502(90.55); 2.498 (68.47); 2.329(0.55); 2.324(0.43); 2.074(2.06); 0.146(0.36); 0.008(3.11); 0(78.62); −0.008(3.99); −0.15(0.38) |
| 322 | 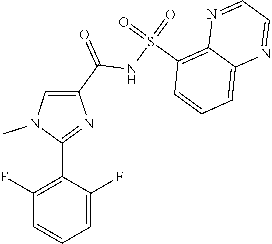 [SMILES: Cn1cc(C(=O)NS(=O)(=O)c2cccc3nccnc23)nc1-c1c(F)cccc1F] | Example 322: ¹H-NMR(400.0 MHz, d₆-DMSO): δ = 9.089(3.73); 9.052(5.01); 9.048(3.97); 8.598(1.92); 8.58(2.08); 8.464(1.47); 8.443(1.63); 8.203(1.74); 8.103(1.35); 8.084(2.18); 8.064(1.17); 7.741 (0.37); 7.723(0.98); 7.703(1.74); 7.686(1.12); 7.666(0.47); 7.353(2.83); 7.333(4.78); 7.313(2.46); 3.54(16); 3.33(5.97); 2.671(1.28); 2.506(182.76); 2.502(216.59); 2.498(162.33); 2.329(1.29); 0.146(0.63); 0(138.14); −0.15 (0.65) |
| 323 | 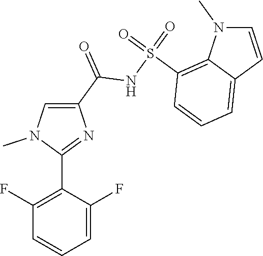 [SMILES: Cn1cc(C(=O)NS(=O)(=O)c2cccc3ccn(C)c23)nc1-c1c(F)cccc1F] | Example 323: ¹H-NMR(400.0 MHz, d₆-DMSO): δ = 8.176(5.73); 7.914(2.14); 7.895(2.23); 7.877(2.05); 7.86(2.17); 7.749(0.33); 7.732(0.73); 7.728(0.74); 7.711(1.41); 7.694(0.77); 7.69(0.88); 7.674(0.38); 7.432(3.01); 7.424(3.14); 7.359(2.44); 7.339(3.69); 7.319(2.09); 7.211 (1.77); 7.192(3.35); 7.172(1.64); 6.652(3.6); 6.644(3.62); 4.18(16); 3.565 (13.18); 3.387(0.97); 3.186(0.41); 2.675(0.42); 2.67(0.57); 2.666(0.44); 2.524(1.58); 2.51(35.62); 2.506(73.44); 2.501(99.21); 2.497(75.09); 2.493 (39.17); 2.333(0.44); 2.328(0.6); 2.324(0.47); 2.074(1.35); 0.146(0.33); 0.008(3.07); 0(78.41); −0.008(4.04); −0.15(0.35) |
| 324 | 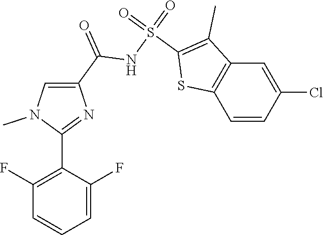 [SMILES: Cc1c(S(=O)(=O)NC(=O)c2cn(C)c(-c3c(F)cccc3F)n2)sc2ccc(Cl)cc12] | Example 324: ¹H-NMR(400.0 MHz, d₆-DMSO): δ = 8.198(3.58); 8.127(2.69); 8.105(2.97); 8.051(2.82); 8.046(2.93); 7.737(0.7); 7.733(0.68); 7.716(1.3); 7.699(0.73); 7.695(0.81); 7.678(0.35); 7.615(1.73); 7.61(1.68); 7.593(1.56); 7.588(1.57); 7.362(2.26); 7.341(3.61); 7.321(1.94); 3.688(0.72); 3.629(1.1); 3.61(1.12); 3.576(13.4); 3.502(1.41); 3.185(0.39); 2.676(16); 2.506(135.77); 2.501(179.19); 2.497(133.48); 2.328(1.09); 0.145 (0.61); 0.007(5.76); −0.001(135.46); −0.009(6.21); −0.15(0.61) |
| 325 | 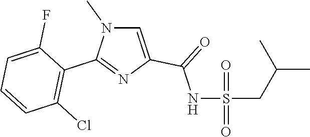 [SMILES: CC(C)CS(=O)(=O)NC(=O)c1cn(C)c(-c2c(F)cccc2Cl)n1] | Example 325: ¹H-NMR(400.0 MHz, d₆-DMSO): δ = 8.248(5); 7.714(0.6); 7.698(0.67); 7.693(1.35); 7.677(1.4); 7.672(0.96); 7.656(0.91); 7.574(2.09); 7.553(1.49); 7.489(0.92); 7.486(0.93); 7.466 (1.55); 7.445(0.78); 7.443(0.76); 3.627(0.54); 3.539(12.82); 3.399(4.42); 3.383(4.64); 3.359(0.58); 3.346(0.67); 3.33(0.75); 2.524(0.33); 2.519(0.51); 2.51(8.89); 2.506(19.27); 2.501(27.38); 2.497(20.25); 2.492(9.5); 2.191 (0.41); 2.174(0.83); 2.158(1.06); 2.141(0.86); 2.124(0.43); 1.044(15.72); 1.027(16); 1.01(0.63); 0.008(0.34); 0(11.69); −0.008(0.37) |
| 327 | 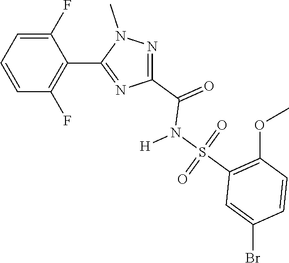 [SMILES: COc1ccc(Br)cc1S(=O)(=O)NC(=O)c1nc(-c2c(F)cccc2F)n(C)n1] | Example 327: ¹H-NMR(400.0 MHz, d₆-DMSO): δ = 8.096(4.56); 7.602(4.37); 7.594(3.01); 7.563(0.63); 7.558(1.11); 7.554 (0.65); 7.545(0.91); 7.54(1.65); 7.536(0.96).7.498(2.62).7.483(0.86); 7.476 (3.14); 7.464(2.04); 7.445(1.83); 7.438(2.01); 7.423(0.41); 7.419(0.59); 7.206(1.49); 7.198(1.45); 7.184(1.3); 7.176(1.26); 4.038(0.44); 4.02(0.47); 3.836(16); 3.784(12.65); 3.444(14.37); 2.995(0.82); 2.977(1.09); 2.96 (0.83); 2.943(0.35); 2.671(0.45); 2.666(0.33); 2.524(0.93); 2.52(1.48); 2.511 (27.01); 2.506(58.63); 2.502(80.2); 2.497(57.98); 2.493(27.59); 2.333(0.34); 2.328(0.47); 2.324(0.35); 1.988(1.99); 1.511(0.39); 1.254(14.77); 1.237 (14.58); 1.193(0.55); 1.175(1.04); 1.157(0.51); 0.008(0.66); 0(21.32); −0.008(0.75) |
| 328 | 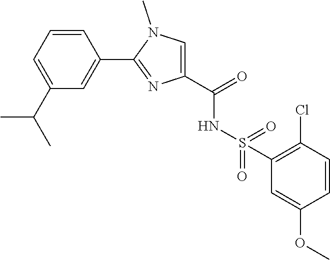 [SMILES: COc1ccc(Cl)c(S(=O)(=O)NC(=O)c2cn(C)c(-c3cccc(C(C)C)c3)n2)c1] | Example 328: ¹H-NMR(400.0 MHz, d₆-DMSO): δ = 8.137(1.41); 8.133(1.68); 8.118(1.61); 8.114(1.76); 8.104(4.95); 7.653 (0.35); 7.649(0.39); 7.633(1.15); 7.629(1.17); 7.616(1.87); 7.612(2.19); 7.608(2.91); 7.604(4.82); 7.589(1.06); 7.585(0.58); 7.577(1.28); 7.572 (0.99); 7.556(2.39); 7.553(1.79); 7.541(1.63); 7.538(2.11); 7.479(0.95); 7.464(0.71); 7.459(2.47); 7.441(1.88); 7.436(1.46); 7.432(2.44); 7.417 (0.53); 7.412(0.9); 4.038(0.39); 4.02(0.41); 3.782(14.31); 3.446(22.5); 3.011(0.38); 2.994(0.9); 2.977(1.22); 2.96(0.93); 2.942(0.39); 2.671(0.43); 2.524(1.08); 2.511(25.99); 2.507(55.45); 2.502(75.36); 2.498(54.62); 2.493 (26.36); 2.329(0.43); 2.324(0.35); 1.988(1.79); 1.518(0.38); 1.254(16); 1.236(15.8); 1.193(0.46); 1.175(0.91); 1.157(0.45); 0.008(0.6); 0(17.3); −0.008(0.68) |
| 329 | 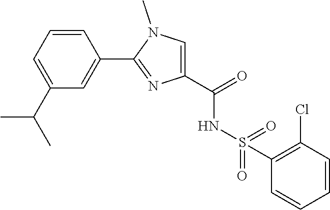 [SMILES: CC(C)c1cccc(-c2nc(C(=O)NS(=O)(=O)c3ccccc3Cl)cn2C)c1] | Example 329: ¹H-NMR(400.0 MHz, d₆-DMSO): δ = 8.831(1.09); 8.807(1.12); 7.982(5.94); 7.806(0.89); 7.788(0.96); 7.659 (0.98); 7.657(0.91); 7.639(2.33); 7.636(1.49); 7.608(0.9); 7.603(1.12); 7.59 (1.18); 7.585(2.76); 7.566(3.21); 7.564(2.55); 7.515(1.29); 7.511(1.29); 7.495(1.6); 7.491(0.8); 7.478(0.58); 7.474(0.57); 7.291(1.07); 7.28(2.32); 7.275(4.5); 7.259(1.35); 7.243(0.34); 6.225(0.62); 6.202(0.91); 6.179(0.65); 4.038(0.41); 4.021(0.43); 3.5(13.68); 3.32(30.62); 2.524(0.58); 2.511 (13.01); 2.506(27.21); 2.502(36.37); 2.497(26.06); 2.493(12.38); 2.428 (11.23); 1.988(1.89); 1.397(16); 1.193(0.5); 1.175(0.99); 1.157(0.48); 0.008 (2.06); 0(54.32); −0.008(2.12) |
| 329 | 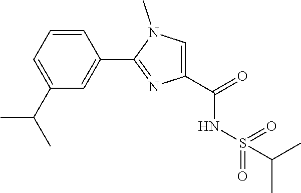 [SMILES: CC(C)c1cccc(-c2nc(C(=O)NS(=O)(=O)C(C)C)cn2C)c1] | Example 329: ¹H-NMR(400.0 MHz, d₆-DMSO): δ = 8.142(4.94); 7.619(2.38); 7.569(0.79); 7.566(1.25); 7.55(1.03); 7.547(1.6); 7.463(1.06); 7.444(2.37); 7.425(1.5); 7.4(1.76); 7.384(0.6); 7.38(0.86); 6.634(0.59); 3.81(0.81); 3.798(14.33); 3.776(1.7); 3.769(0.81); 3.758(1.29); 3.741(0.65); 3.724(0.38); 3.652(0.57); 3.619(0.8); 3.415(15.45); 3.352 (4.33); 3.173(0.47); 3.049(0.35); 3.032(0.62); 3.016(0.91); 2.999(1.17); 2.983(1.35); 2.966(1.01); 2.949(0.48); 2.676(0.36); 2.671(0.52); 2.667 (0.38); 2.525(1.24); 2.511(29.52); 2.507(61.92); 2.502(82.91); 2.498(59.15); 2.493(27.83); 2.334(0.36); 2.329(0.49); 2.324(0.35); 1.526(1.12); 1.319 (13.51); 1.302(13.37); 1.262(16); 1.244(15.83); 1.233(6.14); 1.216(5.73); 1.131(3.95); 1.114(3.9); 0.008(0.7); 0(19.68); −0.008(0.72) |
| 330 | 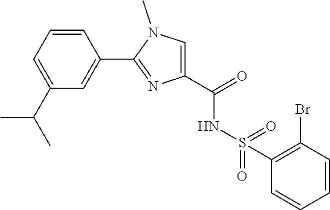 [SMILES: CC(C)c1cccc(-c2nc(C(=O)NS(=O)(=O)c3ccccc3Br)cn2C)c1] | Example 330: ¹H-NMR(400.0 MHz, d₆-DMSO): δ = 8.165(1.47); 8.161(1.64); 8.146(1.65); 8.141(1.73); 8.108(4.99); 7.794 (1.51); 7.791(1.7); 7.774(1.82); 7.771(1.96); 7.621(0.73); 7.618(0.85); 7.602(3.62); 7.583(1.13); 7.58(1.12); 7.564(0.72); 7.56(1.27); 7.542(2.49); 7.524(1.43); 7.52(1.52); 7.505(0.64); 7.501(0.61); 7.479(0.98); 7.46(2.46); 7.441(1.85); 7.436(1.47); 7.432(2.3); 7.413(0.74); 3.784(14.44); 3.438 (21.59); 3.012(0.37); 2.995(0.91); 2.978(1.23); 2.961(0.96); 2.943(0.39); 2.675(0.33); 2.671(0.46); 2.666(0.35); 2.524(0.9); 2.511(27.46); 2.506 (59.75); 2.502(82.05); 2.497(60.26); 2.493(29.35); 2.333(0.33); 2.328(0.48); 2.324(0.37); 1.988(1.16); 1.254(16); 1.237(15.82); 1.193(0.32); 1.175(0.59); 1.07(0.37); 0.008(0.52); 0(18.18); −0.008(0.68) |
| 331 | 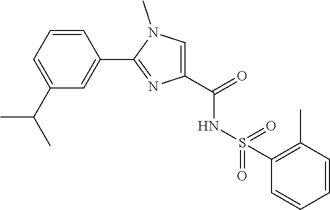 [SMILES: Cc1ccccc1S(=O)(=O)NC(=O)c1cn(C)c(-c2cccc(C(C)C)c2)n1] | Example 331: ¹H-NMR(400.0 MHz, d₆-DMSO): δ = 8.046(5.63); 8.019(1.57); 8.002(1.59); 7.999(1.63); 7.592(2.6); 7.567(0.68); 7.548(2.3); 7.53(2.1); 7.527(2.15); 7.458(1.09); 7.44(3.32); 7.421(3.04); 7.399(2.2); 7.385(1.92); 7.367(1.43); 3.763(14.41); 3.408(39.13); 3.008 (0.43); 2.992(0.97); 2.974(1.29); 2.957(1); 2.94(0.44); 2.676(0.49); 2.671 (0.67); 2.667(0.49); 2.612(11.69); 2.511(39.94); 2.507(83.14); 2.502 (111.56); 2.498(81.08); 2.493(39.35); 2.333(0.46); 2.329(0.65); 2.324(0.49); 1.511(0.71); 1.254(16); 1.237(15.75); 0.008(0.89); 0(23.18); −0.008(0.83) |
| 332 | 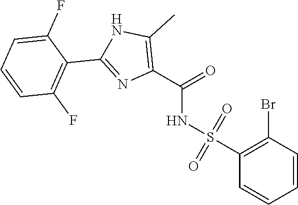 [SMILES: Cc1[nH]c(-c2c(F)cccc2F)nc1C(=O)NS(=O)(=O)c1ccccc1Br] | Example 332: ¹H-NMR(400.0 MHz, d₆-DMSO): δ = 13.306(0.52); 8.188(1.92); 8.184(2.07); 8.169(2.13); 8.165(2.18); 7.844 (2.03); 7.824(2.34); 7.665(0.96); 7.662(1); 7.646(2.83); 7.627(2.22); 7.613 (0.99); 7.609(1.1); 7.599(1.37); 7.595(1.59); 7.58(1.72); 7.576(1.77); 7.561 (0.78); 7.557(0.69); 7.337(0.51); 7.329(2.97); 7.309(5.08); 7.288(2.49); 7.28 (0.52); 7.064(0.46); 4.269(0.45); 4.255(0.48); 3.849(0.65); 2.675(0.57); 2.67 (0.8); 2.666(0.59); 2.524(1.93); 2.51(46.71); 2.506(96.94); 2.501(135.22); 2.497(102.92); 2.493(51.57); 2.427(16); 2.333(0.66); 2.328(0.87); 2.324 (0.66); 0.146(0.77); 0.008(5.75); 0(166.56); −0.008(7.18); −0.15(0.78) |
| 333 | 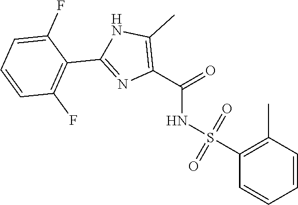 [SMILES: Cc1ccccc1S(=O)(=O)NC(=O)c1nc(-c2c(F)cccc2F)[nH]c1C] | Example 333: ¹H-NMR(400.0 MHz, d₆-DMSO): δ = 13.1(0.95); 8.037(2.13); 8.017(2.26); 7.647(0.39); 7.631(0.86); 7.626(0.84); 7.61(1.64); 7.594(0.95); 7.589(1.14); 7.584(0.95); 7.572(0.68); 7.564(1.94); 7.544(1.28); 7.454(1.23); 7.434(1.95); 7.416(0.91); 7.399(2.2); 7.38(1.86); 7.32(0.51); 7.312(2.93); 7.292(4.96); 7.271(2.49); 7.263(0.53); 7.247(0.36); 7.083(0.34); 7.063(0.52); 4.269(0.36); 4.254(0.36); 3.846(0.39); 3.634 (0.63); 3.595(0.72); 3.445(0.94); 3.274(0.54); 2.675(0.61); 2.67(0.82); 2.666 (0.63); 2.614(16); 2.524(1.94); 2.506(97.64); 2.501(135.94); 2.497(103.98); 2.493(52.59); 2.451(0.36); 2.399(15.94); 2.333(0.66); 2.328(0.87); 2.324 (0.68); 0.146(0.74); 0.008(5.43); 0(156.84); −0.008(6.64); −0.15(0.77) |
| 334 | 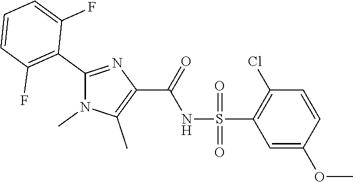 [SMILES: COc1ccc(Cl)c(S(=O)(=O)NC(=O)c2nc(-c3c(F)cccc3F)n(C)c2C)c1] | Example 334: ¹H-NMR(400.0 MHz, d₆-DMSO): δ = 7.74(0.63); 7.736(0.61); 7.719(1.2); 7.702(0.66); 7.698(0.74); 7.604(2.85); 7.596(3.04); 7.566(2.29); 7.544(2.67); 7.367(2.1); 7.346(3.28); 7.326(1.83); 7.282(1.35); 7.274(1.31); 7.26(1.18); 7.252(1.15); 3.856(16); 3.423(10.32); 2.675(0.37); 2.67(0.5); 2.666(0.38); 2.523(1.28); 2.506(60.22); 2.501 (82.92); 2.497(63.33); 2.47(14.22); 2.332(0.39); 2.328(0.51); 2.324(0.39); 0.008(2.27); 0(54.31); −0.008(2.4) |
| 335 | 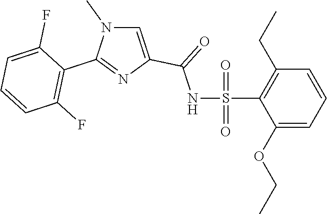 [SMILES: CCOc1cccc(CC)c1S(=O)(=O)NC(=O)c1cn(C)c(-c2c(F)cccc2F)n1] | Example 335: ¹H-NMR(400.0 MHz, d₆-DMSO): δ = 10.387(0.39); 8.184(6.33); 7.756(0.39); 7.739(0.99); 7.718(1.74); 7.7(1.07); 7.698(1.07); 7.681(0.44); 7.512(1.5); 7.492(2.98); 7.472(1.79); 7.372(2.94); 7.351(4.9); 7.331(2.54); 7.056(2.71); 7.035(2.49); 6.977(2.82); 6.958(2.63); 4.1(1.37); 4.083(4.36); 4.066(4.41); 4.048(1.41); 3.574(16); 3.319(10.32); 3.136(1.21); 3.117(3.8); 3.099(3.9); 3.08(1.26); 2.671(0.36); 2.502(60.11); 2.329(0.35); 2.073(2.53); 1.283(4.57); 1.265(12.23); 1.245(11.12); 1.226 (4.24); 0(60.44) |
| 336 | 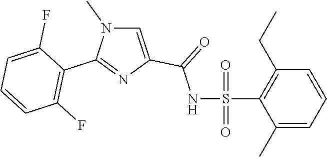 [SMILES: CCc1cccc(C)c1S(=O)(=O)NC(=O)c1cn(C)c(-c2c(F)cccc2F)n1] | Example 336: ¹H-NMR(400.0 MHz, d₆-DMSO): δ = 8.169(6.66); 7.738(0.4); 7.722(1.01); 7.717(0.95); 7.7(1.77); 7.683(1.07); 7.68(1.08); 7.663(0.44); 7.453(1.34); 7.434(3.15); 7.415(2.03); 7.348(2.96); 7.328(4.74); 7.308(2.54); 7.276(2.47); 7.258(1.99); 7.218(2.36); 7.199 (2.04); 3.56(15.91); 3.381(0.63); 3.328(0.62); 3.304(0.61); 3.292(0.58); 3.206(0.38); 3.162(1.41); 3.143(4.01); 3.125(4.08); 3.106(1.46); 2.685(16); 2.505(49.17); 2.501(63.29); 2.497(50.13); 2.328(0.36); 2.073(0.66); 1.236 (4.74); 1.192(4.35); 1.173(9.37); 1.155(4.3); 0.854(0.43); 0.146(0.32); 0 (65.33) |
| 337 | 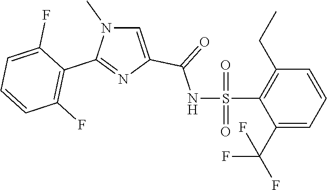 [SMILES: CCc1cccc(C(F)(F)F)c1S(=O)(=O)NC(=O)c1cn(C)c(-c2c(F)cccc2F)n1] | Example 337: ¹H-NMR(400.0 MHz, d₆-DMSO): δ = 8.163(7.03); 7.856(1.28); 7.85(1.29); 7.839(1.91); 7.833(2.1); 7.783(0.9); 7.769(4.83); 7.766(4.82); 7.749(1.99); 7.736(1.03); 7.731(1.21); 7.715 (1.77); 7.698(0.94); 7.694(1.05); 7.677(0.45); 7.366(0.57); 7.359(3.13); 7.339(4.78); 7.319(2.66); 7.312(0.55); 3.576(16); 3.219(1.24); 3.201(4.03); 3.182(4.1); 3.164(1.32); 2.671(0.43); 2.524(0.9); 2.511(24.4); 2.506(51.18); 2.502(71.36); 2.497(53.75); 2.493(26.39); 2.333(0.34); 2.329(0.44); 2.324 (0.33); 2.073(8.96); 1.162(4.45); 1.143(9.98); 1.125(4.42); 0.146(0.41); 0.008(2.97); 0(93.28); −0.008(3.65); −0.15(0.44) |
| 338 | 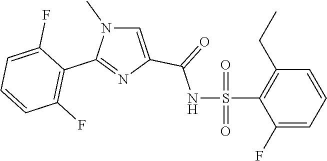 [SMILES: CCc1cccc(F)c1S(=O)(=O)NC(=O)c1cn(C)c(-c2c(F)cccc2F)n1] | Example 338: ¹H-NMR(400.0 MHz, d₆-DMSO): δ = 8.22(7.2); 7.743(0.41); 7.726(0.94); 7.722(0.91); 7.705(1.76); 7.688(0.94); 7.684(1.09); 7.667(0.45); 7.64(0.76); 7.627(0.89); 7.62(1.61); 7.607(1.63); 7.6(1.07); 7.587(0.92); 7.353(3.06); 7.333(4.7); 7.313(2.62); 7.282(2.47); 7.262(2.43); 7.237(1.21); 7.23(1.4); 7.209(1.1); 3.572(16); 3.392(0.37); 3.172(1.36); 3.154(4.03); 3.135(4.12); 3.116(1.42); 2.675(0.34); 2.671 (0.45); 2.666(0.36); 2.524(1.11); 2.51(26.57); 2.506(54.03); 2.502(74.39); 2.497(57.74); 2.333(0.36); 2.328(0.48); 2.324(0.37); 2.073(1.11); 1.248 (4.67); 1.23(10.32); 1.211(4.61); 0.146(0.42); 0.008(3.78); 0(91.41); −0.008 (4.82); −0.15(0.42) |
| 339 | 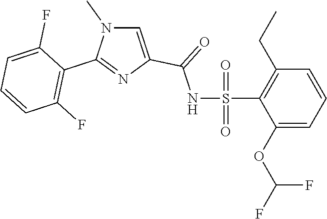 [SMILES: CCc1cccc(OC(F)F)c1S(=O)(=O)NC(=O)c1cn(C)c(-c2c(F)cccc2F)n1] | Example 339: ¹H-NMR(400.0 MHz, d₆-DMSO): δ = 8.207(6.82); 7.746(0.43); 7.729(0.95); 7.724(0.91); 7.708(1.8); 7.691(0.94); 7.687(1.08); 7.67(0.48); 7.645(1.65); 7.625(3.47); 7.605(2.12); 7.403(1.39); 7.364(0.56); 7.357(3.26); 7.337(5.41); 7.321(2.7); 7.317(3.04); 7.309(0.57); 7.219(4.67); 7.201(2.02); 7.035(1.47); 3.575(16); 3.201(1.21); 3.183(3.85); 3.164(3.95); 3.145(1.3); 2.524(0.59); 2.519(0.9); 2.511(15.62); 2.506 (33.87); 2.502(48.2); 2.497(36.38); 2.493(17.69); 2.073(0.45); 1.251(4.52); 1.232(10.25); 1.214(4.5); 0.008(1.68); 0(56.32); −0.008(2.08) |
| 340 | 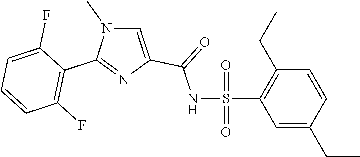 [SMILES: CCc1ccc(CC)c(S(=O)(=O)NC(=O)c2cn(C)c(-c3c(F)cccc3F)n2)c1] | Example 340: ¹H-NMR(400.0 MHz, d₆-DMSO): δ = 8.174(7.21); 7.852(3.49); 7.848(3.75); 7.739(0.43); 7.722(0.95); 7.718 (0.92); 7.701(1.77); 7.684(0.94); 7.68(1.09); 7.663(0.46); 7.476(1.47); 7.472(1.53); 7.457(2.05); 7.452(2.12); 7.358(4.14); 7.349(3.34); 7.338 (3.27); 7.329(4.76); 7.309(2.66); 7.301(0.53); 3.562(16); 3.382(0.55); 3.355(0.49); 3.307(0.46); 3.032(1.26); 3.013(3.91); 2.995(3.98); 2.976 (1.32); 2.703(1.26); 2.684(3.9); 2.665(4.12); 2.646(1.37); 2.524(0.73); 2.511(16.26); 2.506(34.4); 2.502(48.42); 2.497(37.44); 2.493(18.97); 2.073 (0.8); 1.22(5.74); 1.202(12.34); 1.183(5.6); 1.157(4.81); 1.138(10.56); 1.12 (4.73); 0.008(2.23); 0(68.04); −0.008(2.89); −0.15(0.32) |
| 341 | 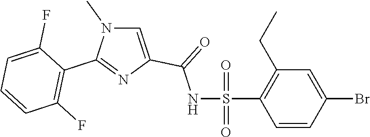 [SMILES: CCc1cc(Br)ccc1S(=O)(=O)NC(=O)c1cn(C)c(-c2c(F)cccc2F)n1] | Example 341: ¹H-NMR(400.0 MHz, d₆-DMSO): δ = 8.177(6.8); 7.943(3.12); 7.922(3.65); 7.745(0.38); 7.728(0.9); 7.724(0.88); 7.707(1.66); 7.69(0.96); 7.687(1.05); 7.67(8.2); 7.648(2.11); 7.644(1.54); 7.353(2.87); 7.333(4.73); 7.313(2.49); 3.568(16); 3.389(0.38); 3.06(1.26); 3.042(3.83); 3.023(3.92); 3.005(1.33); 2.673(0.36); 2.506(44.03); 2.502 (59.19); 2.499(46.59); 2.329(0.4); 2.074(1.7); 1.172(4.31); 1.153(9.25); 1.134(4.25); 0.008(2.29); 0.001(55.46); 0(63.61) |
| 342 | 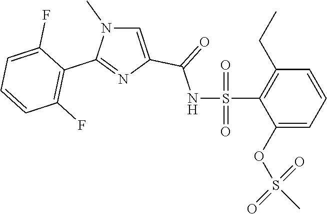 [SMILES: CCc1cccc(OS(C)(=O)=O)c1S(=O)(=O)NC(=O)c1cn(C)c(-c2c(F)cccc2F)n1] | Example 342: ¹H-NMR(400.0 MHz, d₆-DMSO): δ = 8.159(3.72); 7.735(0.58); 7.73(0.57); 7.714(1.11); 7.697(0.58); 7.692(0.71); 7.663(0.96); 7.643(2.17); 7.623(1.37); 7.413(1.72); 7.392(2.63); 7.371 (1.67); 7.369(1.67); 7.361(2.12); 7.341(2.94); 7.321(1.67); 3.707(0.37); 3.573(10.26); 3.533(16); 3.458(0.62); 3.393(0.6); 3.355(0.55); 3.211(0.95); 3.192(2.54); 3.174(2.57); 3.155(0.95); 2.67(0.44); 2.666(0.34); 2.524(0.89); 2.519(1.36); 2.51(21.9); 2.506(48.28); 2.501(69.98); 2.497(55.8); 2.492 (29.78); 2.333(0.32); 2.328(0.45); 2.324(0.35); 2.073(2.48); 1.244(2.81); 1.225(6.4); 1.206(2.82); 0.146(0.36); 0.008(2.49); 0(81.92); −0.008(5.99); −0.15(0.36) |
| 343 | 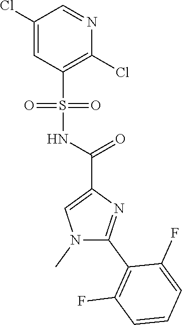 [SMILES: Cn1cc(C(=O)NS(=O)(=O)c2cc(Cl)cnc2Cl)nc1-c1c(F)cccc1F] | Example 343: ¹H-NMR(400.0 MHz, d₆-DMSO): δ = 8.739(5.42); 8.733(5.7); 8.594(0.36); 8.587(0.37); 8.494(5.9); 8.487(5.61); 8.272(8.36); 8.053(0.38); 8.047(0.37); 7.82(0.43); 7.803(0.96); 7.799(0.91); 7.782(1.78); 7.765(0.96); 7.761(1.06); 7.744(0.47); 7.413(2.99); 7.392 (5.16); 7.372(2.58); 4.788(0.38); 4.743(0.52); 4.64(0.74); 3.658(16); 3.583 (0.51); 2.546(78.94); 2.529(0.39); 2.524(0.41); 2.516(5.71); 2.511(12.34); 2.507(17.41); 2.502(12.94); 2.498(6.13); 0(0.37) |
| 344 | 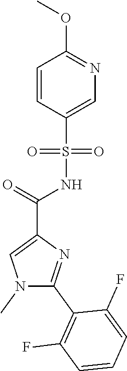 [SMILES: COc1ccc(S(=O)(=O)NC(=O)c2cn(C)c(-c3c(F)cccc3F)n2)cn1] | Example 344: ¹H-NMR(400.0 MHz, d₆-DMSO): δ = 8.755(2.62); 8.749(2.6); 8.221(1.72); 8.215(1.67); 8.199(1.81); 8.192(1.88); 8.182(5.58); 7.73(0.64); 7.725(0.61); 7.708(1.19); 7.692(0.63); 7.687(0.71); 7.363(0.41); 7.356(2.08); 7.336(3.2); 7.316(1.76); 7.308(0.34); 7.05(2.66); 7.028(2.59); 3.954(16); 3.564(10.85); 2.544(9); 2.514(5.28); 2.509(11.15); 2.505(15.68); 2.5(11.87); 2.496(5.83) |
| 345 | 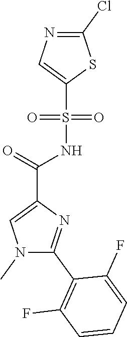 [SMILES: Cn1cc(C(=O)NS(=O)(=O)c2cnc(Cl)s2)nc1-c1c(F)cccc1F] | Example 345: ¹H-NMR(400.0 MHz, d₆-DMSO): δ = 8.235(3.74); 8.132(5.94); 7.819(0.34); 7.802(0.71); 7.798(0.71); 7.781 (1.34); 7.764(0.73); 7.76(0.81); 7.744(0.34); 7.414(2.36); 7.393(4.04); 7.372(2.03); 3.707(0.49); 3.648(12.87); 3.518(16); 2.676(0.74); 2.671 (1.02); 2.666(0.76); 2.541(62.8); 2.524(2.61); 2.52(3.8); 2.511(56.01); 2.506(119.66); 2.502(168.07); 2.497(124.56); 2.493(59.25); 2.333(0.7); 2.329(0.95); 2.324(0.69); 1.236(0.36); 0(1.09) |
| 346 | 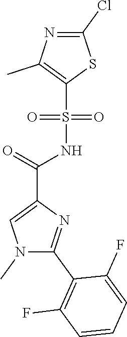 [SMILES: Cc1nc(Cl)sc1S(=O)(=O)NC(=O)c1cn(C)c(-c2c(F)cccc2F)n1] | Example 346: ¹H-NMR(400.0 MHz, d₆-DMSO): δ = 8.25(5.36); 7.778(0.61); 7.774(0.58); 7.757(1.13); 7.74(0.61); 7.736(0.67); 7.396(1.97); 7.375(3.27); 7.354(1.68); 3.628(10.57); 2.563(16); 2.543 (25.42); 2.513(6.83); 2.508(14.14); 2.504(19.93); 2.499(14.93); 2.495(7.26) |
| 347 | 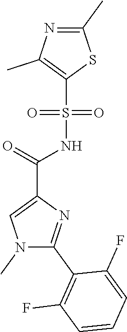 [SMILES: Cc1nc(C)c(S(=O)(=O)NC(=O)c2cn(C)c(-c3c(F)cccc3F)n2)s1] | Example 347: ¹H-NMR(400.0 MHz, d₆-DMSO): δ = 8.21(5.08); 7.735(0.67); 7.731(0.66); 7.714(1.29); 7.697(0.67); 7.693(0.78); 7.676(0.33); 7.369(0.4); 7.362(2.23); 7.342(3.5); 7.321(1.93); 3.853(0.55); 3.755(0.85); 3.699(0.89); 3.688(0.9); 3.682(0.9); 3.633(0.85); 3.58(12.7); 3.452(0.37); 3.4(0.33); 2.672(0.38); 2.667(0.34); 2.65(15.31); 2.568(16); 2.542(37.91); 2.525(0.72); 2.511(14.61); 2.507(31.11); 2.502(43.83); 2.498 (32.79); 2.494(15.85) |
| 348 | 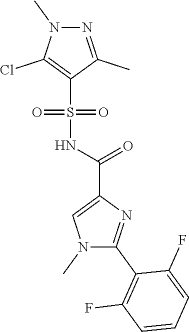 [SMILES: Cc1nn(C)c(Cl)c1S(=O)(=O)NC(=O)c1cn(C)c(-c2c(F)cccc2F)n1] | Example 348: ¹H-NMR(400.0 MHz, d₆-DMSO): δ = 8.214(5.62); 7.725(0.65); 7.72(0.62); 7.703(1.22); 7.686(0.63); 7.682(0.73); 7.36(0.36); 7.353(2.11); 7.333(3.17); 7.312(1.79); 7.305(0.35); 3.779(16); 3.57(11.04); 2.542(12.82); 2.521(0.34); 2.512(5.4); 2.508(11.89); 2.503 (17.07); 2.499(12.87); 2.494(6.22); 2.373(15.54) |
| 349 | 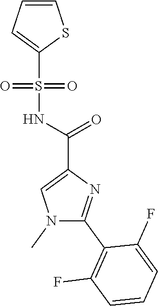 [SMILES: Cn1cc(C(=O)NS(=O)(=O)c2cccs2)nc1-c1c(F)cccc1F] | Example 349: ¹H-NMR(400.0 MHz, d₆-DMSO): δ = 8.21(6.37); 8.034(2.08); 8.03(2.24); 8.021(2.2); 8.018(2.24); 7.83(2.18); 7.827(2.31); 7.821(2.38); 7.817(2.3); 7.752(0.37); 7.735(0.86); 7.731(0.81); 7.714(1.59); 7.697(0.86); 7.693(0.96); 7.676(0.41); 7.369(0.54); 7.362(2.8); 7.342(4.34); 7.322(2.38); 7.314(0.48); 7.218(2.17); 7.208(2.41); 7.206 (2.52); 7.196(2.06); 3.919(0.42); 3.846(0.51); 3.75(0.88); 3.678(1.13); 3.575(16); 3.396(0.7); 3.293(0.38); 2.678(0.36); 2.548(43.21); 2.531(0.86); 2.513(37.32); 2.509(51.56); 2.504(39.25) |
| 350 | 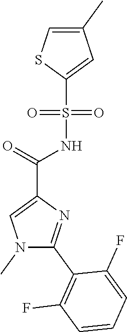 [SMILES: Cc1csc(S(=O)(=O)NC(=O)c2cn(C)c(-c3c(F)cccc3F)n2)c1] | Example 350: ¹H-NMR(400.0 MHz, d₆-DMSO): δ = 8.208(5.84); 7.752(0.39); 7.735(0.86); 7.731(0.82); 7.714(1.61); 7.697 (0.85); 7.693(0.97); 7.676(0.45); 7.655(3.23); 7.651(3.89); 7.617(3.03); 7.37(0.5); 7.363(2.86); 7.343(4.21); 7.322(2.4); 7.315(0.46); 3.751(0.61); 3.576(16); 3.5(1.96); 3.17(0.33); 2.678(0.43); 2.674(0.32); 2.549(50.67); 2.532(0.91); 2.527(1.24); 2.518(22); 2.514(47.69); 2.509(67.52); 2.505 (50.49); 2.501(24.31); 2.341(0.33); 2.336(0.44); 2.332(0.32); 2.248(13.48) |
| 351 | 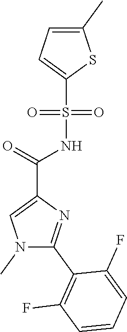 [SMILES: Cc1ccc(S(=O)(=O)NC(=O)c2cn(C)c(-c3c(F)cccc3F)n2)s1] | Example 351: ¹H-NMR(400.0 MHz, d₆-DMSO): δ = 8.191(5.16); 7.743(0.38); 7.726(0.85); 7.722(0.84); 7.705(1.58); 7.688 (0.85); 7.684(0.95); 7.667(0.43); 7.63(3.06); 7.62(3.14); 7.361(0.53); 7.354 (2.71); 7.334(4.23); 7.313(2.31); 7.306(0.44); 6.919(2.13); 6.909(2.05); 3.741(0.47); 3.566(16); 3.415(4.65); 3.411(4.65); 3.407(4.65); 2.994(0.36); 2.675(0.55); 2.671(0.74); 2.667(0.53); 2.541(43.4); 2.524(1.65); 2.511 (46.89); 2.506(77.87); 2.502(108.63); 2.497(82.47); 2.493(40.66); 2.333 (0.49); 2.329(0.67); 2.324(0.5); 0(0.58) |
| 352 | 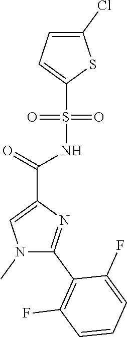 [SMILES: Cn1cc(C(=O)NS(=O)(=O)c2ccc(Cl)s2)nc1-c1c(F)cccc1F] | Example 352: ¹H-NMR(400.0 MHz, d₆-DMSO): δ = 8.227(7.05); 7.764(0.43); 7.747(0.95); 7.742(0.9); 7.726(1.76); 7.709(0.94); 7.704(1.09); 7.688(0.48); 7.67(4.54); 7.66(4.79); 7.377(0.59); 7.37(3.13); 7.35(4.78); 7.33(2.65); 7.323(0.57); 7.266(4.99); 7.255(4.76); 4.248(0.32); 4.193(0.37); 4.132(0.42); 4.124(0.43); 4.088(0.44); 4.078(0.45); 4.071 (0.45); 4.05(0.46); 4.031(0.47); 4.016(0.47); 3.996(0.47); 3.987(0.47); 3.971(0.47); 3.943(0.46); 3.897(0.43); 3.766(0.32); 3.592(16); 2.542(48.34); 2.525(0.56); 2.52(0.74); 2.512(14.97); 2.507(33.27); 2.502(47.82); 2.498 (36.31); 2.494(17.83); 0(0.82) |
| 353 | 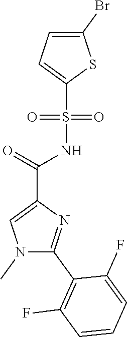 [SMILES: Cn1cc(C(=O)NS(=O)(=O)c2ccc(Br)s2)nc1-c1c(F)cccc1F] | Example 353: ¹H-NMR(400.0 MHz, d₆-DMSO): δ = 8.224(6.86); 7.762(0.43); 7.745(0.93); 7.74(0.91); 7.724(1.73); 7.706(0.95); 7.702(1.06); 7.686(0.45); 7.62(4.13); 7.61(4.49); 7.369(3.13); 7.358(5.3); 7.348(9.08); 7.328(2.65); 7.321(0.61); 4.144(0.36); 4.11(0.38); 4.068(0.41); 3.965(0.47); 3.938(0.47); 3.921(0.49); 3.877(0.5); 3.852(0.49); 3.83(0.48); 3.765(0.45); 3.734(0.35); 3.589(16); 2.542(38.57); 2.525(0.44); 2.511 (15.15); 2.507(33.03); 2.502(47.19); 2.498(36.45); 2.329(0.34); 0(0.76) |
| 354 | 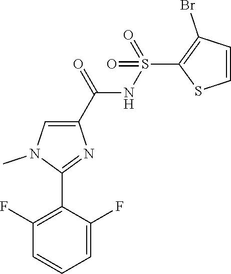 [SMILES: Cn1cc(C(=O)NS(=O)(=O)c2sccc2Br)nc1-c1c(F)cccc1F] | Example 354: ¹H-NMR(400.0 MHz, d₆-DMSO): δ = 8.264(6.09); 8.041(3.52); 8.028(3.67); 7.758(0.39); 7.741(0.97); 7.737 (0.95); 7.72(1.72); 7.703(1.04); 7.699(1.06); 7.682(0.42); 7.367(2.81); 7.347 (4.72); 7.326(2.44); 7.268(3.95); 7.255(3.87); 4.152(0.49); 4.052(0.67); 3.96 (0.81); 3.954(0.82); 3.917(0.86); 3.864(0.89); 3.815(0.85); 3.788(0.79); 3.769(0.79); 3.66(0.39); 3.648(0.36); 3.635(0.34); 3.594(16); 2.542(36.48); 2.502(54.48); 2.498(43.78); 2.329(0.37); 2.325(0.32); 0(0.68) |
| 355 | 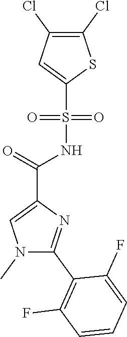 [SMILES: Cn1cc(C(=O)NS(=O)(=O)c2cc(Cl)c(Cl)s2)nc1-c1c(F)cccc1F] | Example 355: ¹H-NMR(400.0 MHz, d₆-DMSO): δ = 8.232(4.39); 7.802(0.32); 7.785(0.72); 7.781(0.73); 7.764(1.35); 7.747 (0.76); 7.743(0.9); 7.728(5.8); 7.401(2.37); 7.38(3.98); 7.359(2.02); 3.631 (16); 3.573(5.12); 2.675(0.57); 2.671(0.79); 2.666(0.6); 2.662(0.32); 2.541 (4.91); 2.524(1.88); 2.511(43.23); 2.506(92.57); 2.502(130.53); 2.497 (97.27); 2.493(46.49); 2.333(0.52); 2.329(0.73); 2.324(0.53); 0(1.88) |
| 356 | 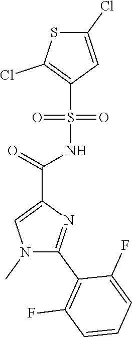 [SMILES: Cn1cc(C(=O)NS(=O)(=O)c2cc(Cl)sc2Cl)nc1-c1c(F)cccc1F] | Example 356: ¹H-NMR(400.0 MHz, d₆-DMSO): δ = 8.248(6.31); 7.77(0.41); 7.753(0.89); 7.748(0.84); 7.732(1.65); 7.715(0.9); 7.71(1.02); 7.694(0.44); 7.422(9.07); 7.377(2.92); 7.356(4.61); 7.336(2.48); 3.777(1.78); 3.725(1.93); 3.626(1.32); 3.603(16); 2.675(0.38); 2.671(0.52); 2.667(0.4); 2.541(52.93); 2.524(1.26); 2.52(1.74); 2.511(26.97); 2.506 (58.36); 2.502(82.99); 2.497(62.86); 2.493(30.76); 2.333(0.33); 2.329 (0.46); 2.324(0.35); 0(1.17) |
| 357 | 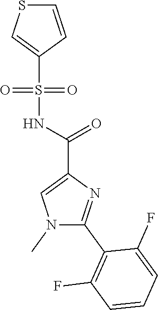 [SMILES: Cn1cc(C(=O)NS(=O)(=O)c2ccsc2)nc1-c1c(F)cccc1F] | Example 357: ¹H-NMR(400.0 MHz, d₆-DMSO): δ = 8.407(2.29); 8.404(2.59); 8.399(2.62); 8.396(2.56); 8.188(7.27); 7.746 (2.35); 7.739(2.47); 7.734(2.73); 7.726(3.45); 7.704(1.79); 7.688(0.97); 7.683(1.09); 7.666(0.46); 7.484(2.8); 7.481(2.9); 7.471(2.61); 7.468(2.67); 7.36(0.64); 7.353(3.12); 7.333(4.71); 7.313(2.67); 7.305(0.57); 3.584(0.61); 3.565(16); 2.671(0.34); 2.541(13.77); 2.524(0.76); 2.51(20.08); 2.506 (43.03); 2.502(60.68); 2.497(46.28); 2.328(0.36); 0.008(0.61); 0(19.94); −0.008(0.86) |
| 358 | 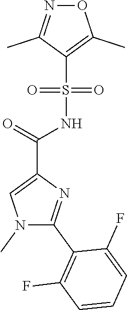 [SMILES: Cc1noc(C)c1S(=O)(=O)NC(=O)c1cn(C)c(-c2c(F)cccc2F)n1] | Example 358: ¹H-NMR(400.0 MHz, d₆-DMSO): δ = 8.223(4.9); 7.735(0.67); 7.731(0.64); 7.714(1.23); 7.698(0.66); 7.693(0.74); 7.676(0.32); 7.369(0.4); 7.362(2.15); 7.342(3.31); 7.321(1.8); 7.314(0.36); 3.875(0.38); 3.754(0.73); 3.58(12.6); 3.4(0.51); 2.686(16); 2.672(0.46); 2.667(0.33); 2.542(21.84); 2.525(0.84); 2.511(17.58); 2.507(37.6); 2.502 (53.01); 2.498(39.83); 2.494(19.27); 2.389(15.85); 0(0.52) |
| 359 | 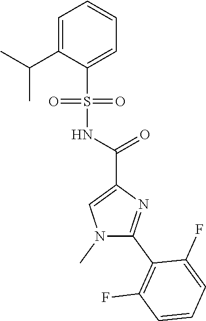 [SMILES: CC(C)c1ccccc1S(=O)(=O)NC(=O)c1cn(C)c(-c2c(F)cccc2F)n1] | Example 359: ¹H-NMR(400.0 MHz, d₆-DMSO): δ = 8.162(5.07); 8.007(2.37); 7.986(2.59); 7.741(0.35); 7.724(0.96); 7.703(1.6); 7.686(1.03); 7.664(0.84); 7.642(1.97); 7.624(1.84); 7.603(2.98); 7.584 (1.35); 7.427(1.34); 7.408(2.06); 7.389(1.1); 7.351(2.63); 7.331(4.46); 7.311 (2.22); 4.009(0.52); 3.993(1.14); 3.976(1.51); 3.959(1.17); 3.943(0.55); 3.738(0.43); 3.717(0.41); 3.562(16); 3.368(9.74); 3.111(0.66); 2.995(0.49); 2.671(0.76); 2.543(36.79); 2.542(45.07); 2.506(102.59); 2.502(119.68); 2.329(0.7); 1.156(14.68); 1.139(14.48); 0.002(1.16); 0(1.46) |
| 360 | 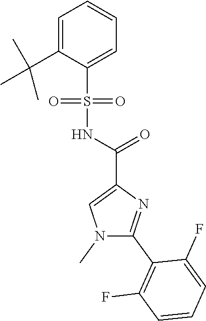 [SMILES: Cn1cc(C(=O)NS(=O)(=O)c2ccccc2C(C)(C)C)nc1-c1c(F)cccc1F] | Example 360: ¹H-NMR(400.0 MHz, d₆-DMSO): δ = 8.189(2.14); 8.171(0.8); 8.168(0.71); 8.151(0.86); 8.148(0.73); 7.74(0.35); 7.719(0.56); 7.702(0.38); 7.697(0.35); 7.687(0.67); 7.667(0.91); 7.592 (0.42); 7.573(0.72); 7.554(0.39); 7.446(0.49); 7.426(0.76); 7.408(0.38); 7.372(0.92); 7.352(1.54); 7.331(0.78); 3.588(5.4); 3.408(1.03); 3.392(1.01); 3.252(0.41); 2.554(3.01); 2.549(11.22); 2.514(14.06); 2.51(17.04); 2.505 (12.04); 1.56(16) |
| 361 | 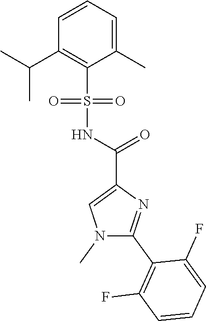 [SMILES: Cc1cccc(C(C)C)c1S(=O)(=O)NC(=O)c1cn(C)c(-c2c(F)cccc2F)n1] | Example 361: ¹H-NMR(400.0 MHz, d₆-DMSO): δ = 8.163(6.19); 7.739(0.38); 7.722(0.91); 7.718(0.91); 7.701(1.68); 7.684 (0.96); 7.68(1.04); 7.663(0.44); 7.484(0.69); 7.464(2.39); 7.447(5.41); 7.428(0.85); 7.349(2.88); 7.329(4.57); 7.308(2.49); 7.197(1.72); 7.193 (1.79); 7.18(1.57); 7.176(1.57); 4.248(0.43); 4.231(1.03); 4.214(1.43); 4.198(1.08); 4.181(0.47); 3.658(0.32); 3.639(0.37); 3.56(16); 3.363(3.69); 2.995(2.17); 2.706(15.96); 2.676(0.49); 2.671(0.51); 2.542(44); 2.506 (50.02); 2.502(67.38); 2.498(52.58); 2.329(0.44); 2.074(2.22); 1.336(0.33); 1.299(0.45); 1.259(0.53); 1.25(0.54); 1.236(1.66); 1.142(14.86); 1.125 (14.91); 0(1.04) |
| 362 | 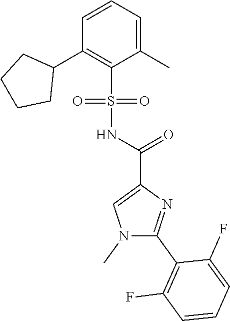 [SMILES: Cc1cccc(C2CCCC2)c1S(=O)(=O)NC(=O)c1cn(C)c(-c2c(F)cccc2F)n1] | Example 362: ¹H-NMR(400.0 MHz, d₆-DMSO): δ = 8.148(6.47); 7.738(0.4); 7.721(0.9); 7.717(0.9); 7.7(1.71); 7.683(0.93); 7.679(1.04); 7.662(0.46); 7.462(0.92); 7.442(2.56); 7.424(2.62); 7.406 (2.76); 7.39(1.1); 7.351(3); 7.331(4.65); 7.311(2.55); 7.175(2.03); 7.159 (1.78); 4.2(0.76); 4.18(1.21); 4.158(0.78); 3.563(16); 3.405(0.79); 3.384 (0.92); 3.356(0.87); 2.705(15.99); 2.541(4.97); 2.506(29.64); 2.502(40.9); 2.498(31.28); 2.074(0.45); 1.958(1.3); 1.945(1.4); 1.814(0.32); 1.796(0.95); 1.777(1.86); 1.744(0.6); 1.682(0.32); 1.673(0.37); 1.652(1.11); 1.641(1.5); 1.635(1.58); 1.623(1.62); 1.491(0.51); 1.465(1.06); 1.449(1.3); 1.444(1.27); 1.423(0.88); 1.402(0.34); 1.258(0.33); 0(0.6) |
| 363 | 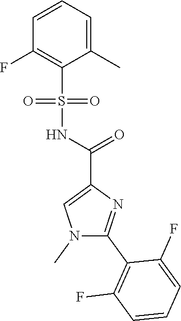 [SMILES: Cc1cccc(F)c1S(=O)(=O)NC(=O)c1cn(C)c(-c2c(F)cccc2F)n1] | Example 363: ¹H-NMR(400.0 MHz, d₆-DMSO): δ = 8.225(6.79); 7.744(0.38); 7.727(0.89); 7.724(0.91); 7.706(1.63); 7.686 (1.02); 7.669(0.42); 7.604(0.7); 7.59(0.86); 7.584(1.48); 7.57(1.54); 7.564 (1.02); 7.55(0.85); 7.356(2.74); 7.335(4.63); 7.315(2.4); 7.27(1.29); 7.252 (3.25); 7.234(2.38); 7.222(1.2); 3.574(15.58); 3(0.47); 2.682(16); 2.543 (32.65); 2.504(13.52); 2.501(11.69); 2.37(0.35); 2.076(0.86); 1.235(0.65) |
| 364 | 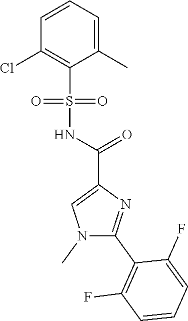 [SMILES: Cc1cccc(Cl)c1S(=O)(=O)NC(=O)c1cn(C)c(-c2c(F)cccc2F)n1] | Example 364: ¹H-NMR(400.0 MHz, d₆-DMSO): δ = 8.235(6.69); 7.742(0.37); 7.725(0.87); 7.721(0.83); 7.704(1.59); 7.688 (0.89); 7.683(0.95); 7.667(0.4); 7.535(0.61); 7.515(2.42); 7.499(5.4); 7.482 (0.82); 7.42(1.78); 7.415(1.7); 7.404(1.23); 7.398(1.2); 7.354(2.72); 7.334 (4.33); 7.313(2.34); 3.572(14.84); 2.997(0.66); 2.743(16); 2.542(30.74); 2.525(0.59); 2.507(19.53); 2.503(26.77); 2.498(20.43); 2.075(0.98); 1.259 (0.4); 1.25(0.4); 1.235(1.25); 0(0.39) |
| 365 | 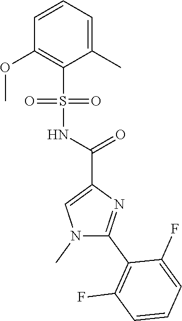 [SMILES: COc1cccc(C)c1S(=O)(=O)NC(=O)c1cn(C)c(-c2c(F)cccc2F)n1] | Example 365: ¹H-NMR(400.0 MHz, d₆-DMSO): δ = 8.206(5.76); 7.729(0.67); 7.725(0.67); 7.708(1.28); 7.691(0.71); 7.687 (0.79); 7.67(0.34); 7.496(1.22); 7.476(2.25); 7.456(1.47); 7.361(2.22); 7.341 (3.49); 7.321(1.89); 7.079(1.87); 7.058(1.69); 6.968(1.84); 6.949(1.73); 3.8 (16); 3.572(11.62); 3.334(2.87); 2.996(0.69); 2.641(12.35); 2.541(24.46); 2.525(0.53); 2.506(16.98); 2.502(23.61); 2.498(18.15); 2.074(0.33); 1.235 (0.85); 0(0.34) |
| 366 | 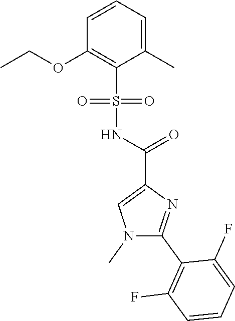 [SMILES: CCOc1cccc(C)c1S(=O)(=O)NC(=O)c1cn(C)c(-c2c(F)cccc2F)n1] | Example 366: ¹H-NMR(400.0 MHz, d₆-DMSO): δ = 8.184(7.38); 7.755(0.39); 7.738(0.88); 7.734(0.87); 7.717(1.67); 7.7(0.9); 7.696(1.02); 7.679(0.44); 7.478(1.55); 7.458(2.87); 7.438(1.89); 7.378 (0.57); 7.371(2.86); 7.351(4.5); 7.33(2.46); 7.064(2.38); 7.043(2.15); 6.956 (2.41); 6.936(2.25); 4.108(1.36); 4.091(4.46); 4.073(4.53); 4.056(1.42); 3.572(15.15); 3.335(3.03); 2.64(16); 2.543(2.01); 2.512(6.99); 2.508(14.56); 2.503(20.36); 2.499(15.67); 1.281(4.63); 1.264(9.77); 1.246(4.67); 1.235 (0.63); 0(0.32) |
| 367 | 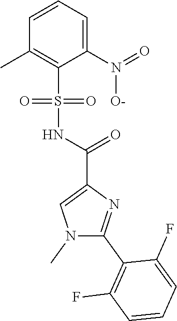 | Example 367: ¹H-NMR(400.0 MHz, d₆-DMSO): δ = 8.373(1.57); 8.211(6.72); 7.784(0.4); 7.767(0.93); 7.763(0.9); 7.746(1.74); 7.729(1); 7.724(1.2); 7.709(1.35); 7.701(0.42); 7.689(2.61); 7.671(3.68); 7.662(2.48); 7.658(3.31); 7.643(1.11); 7.639(0.79); 7.608(2.11); 7.604 (2.04); 7.59(1.42); 7.586(1.34); 7.386(2.93); 7.366(4.92); 7.345(2.77); 7.328 (0.61); 4.297(0.32); 4.278(0.34); 4.26(0.36); 4.224(0.36); 4.197(0.38); 4.126 (0.43); 4.097(0.46); 3.851(0.75); 3.813(0.78); 3.79(0.83); 3.659(0.32); 3.647 (0.41); 3.638(0.34); 3.613(15.08); 3.592(3.9); 2.996(0.67); 2.685(16); 2.542 (41.91); 2.511(14.17); 2.507(29.3); 2.503(40.66); 2.498(30.88); 2.074(1.69); 1.3(0.41); 1.259(0.56); 1.25(0.54); 1.235(2.14); 0(0.63) |
| 368 | 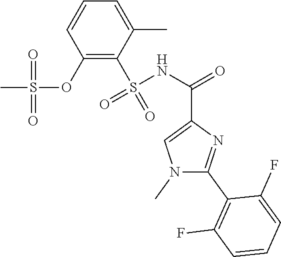 [SMILES: Cc1cccc(OS(C)(=O)=O)c1S(=O)(=O)NC(=O)c1cn(C)c(-c2c(F)cccc2F)n1] | Example 368: ¹H-NMR(400.0 MHz, d₆-DMSO): δ = 8.172(5.55); 7.752(0.32); 7.735(0.72); 7.731(0.69); 7.714(1.27); 7.697 (0.71); 7.693(0.77); 7.676(0.33); 7.63(1.19); 7.61(2.44); 7.59(1.69); 7.407 (2.09); 7.385(3.24); 7.362(3.39); 7.341(3.46); 7.321(1.85); 3.576(11.29); 3.542(16); 2.998(0.33); 2.715(11.4); 2.542(39.31); 2.521(0.5); 2.507(7.28); 2.503(10.25); 2.499(8.12); 2.074(0.88); 1.236(0.54) |
| 369 | 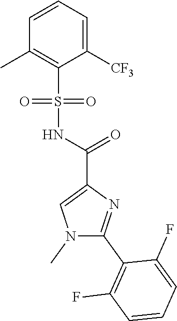 [SMILES: Cc1cccc(C(F)(F)F)c1S(=O)(=O)NC(=O)c1cn(C)c(-c2c(F)cccc2F)n1] | Example 369: ¹H-NMR(400.0 MHz, d₆-DMSO): δ = 8.191(6.82); 7.88(1.19); 7.87(1.9); 7.856(1.57); 7.755(0.42); 7.738(1.02); 7.734(1.08); 7.72(4.79); 7.71(5.97); 7.701(1.29); 7.68(0.39); 7.364(2.63); 7.343(4.3); 7.323(2.24); 3.582(14.46); 2.743(16); 2.544(26.51); 2.528 (0.38); 2.51(9.37); 2.505(13.04); 2.501(10.19); 1.235(0.69) |
| 370 | 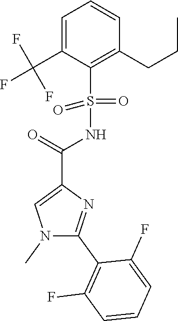 [SMILES: CCCc1cccc(C(F)(F)F)c1S(=O)(=O)NC(=O)c1cn(C)c(-c2c(F)cccc2F)n1] | Example 370: ¹H-NMR(400.0 MHz, d₆-DMSO): δ = 8.17(0.36); 8.147(3.22); 7.849(1.14); 7.844(1.18); 7.832(1.55); 7.826(1.62); 7.761(0.79); 7.745(4.31); 7.728(2.03); 7.714(1.71); 7.697(0.94); 7.693 (0.93); 7.677(0.41); 7.361(2.59); 7.341(4.19); 7.321(2.2); 3.976(0.34); 3.954 (0.37); 3.774(0.88); 3.748(1.05); 3.573(16); 3.536(2.33); 3.419(1.01); 3.395 (0.87); 3.29(0.44); 3.135(2.13); 3.121(1.85); 3.115(2.36); 3.108(1.82); 3.095 (2.25); 2.995(0.85); 2.671(0.42); 2.542(20.07); 2.506(54.89); 2.502(73.2); 2.498(56.91); 2.329(0.49); 2.074(1.71); 1.552(1.02); 1.534(1.7); 1.514 (1.77); 1.495(1.19); 1.477(0.45); 1.336(0.49); 1.299(0.73); 1.259(0.98); 1.249(0.81); 1.235(2.76); 0.933(3.79); 0.915(7.78); 0.897(3.49); 0.79(0.4); 0(0.73) |
| 371 | 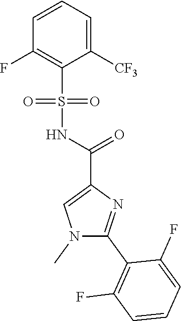 [SMILES: Cn1cc(C(=O)NS(=O)(=O)c2c(F)cccc2C(F)(F)F)nc1-c1c(F)cccc1F] | Example 371: ¹H-NMR(400.0 MHz, d₆-DMSO): δ = 8.233(6.64); 7.927(0.42); 7.907(1.26); 7.895(1.33); 7.887(1.4); 7.866(3.65); 7.849(1.27); 7.798(1.34); 7.773(1.55); 7.769(1.59); 7.749(1.98); 7.729 (1.89); 7.708(1.22); 7.691(0.5); 7.374(3.06); 7.353(5.11); 7.333(2.67); 3.601 (16); 3.575(0.64); 2.671(0.46); 2.541(32.13); 2.502(81.68); 2.329(0.5); 0 (26.92) |
| 372 | 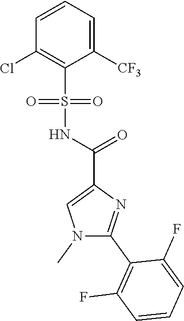 [SMILES: Cn1cc(C(=O)NS(=O)(=O)c2c(Cl)cccc2C(F)(F)F)nc1-c1c(F)cccc1F] | Example 372: ¹H-NMR(400.0 MHz, d₆-DMSO): δ = 8.24(7.08); 8.033(2.26); 8.013(2.67); 7.966(2.2); 7.964(2.27); 7.946(3.06); 7.83(1.8); 7.81(2.81); 7.79(1.18); 7.768(0.45); 7.751(1.02); 7.746(0.99); 7.73(1.85); 7.713(1.04); 7.708(1.12); 7.692(0.48); 7.373(3.18); 7.353(5.16); 7.333(2.73); 3.605(16); 3(0.44); 2.544(47.76); 2.51(10.83); 2.505(14.99); 2.501(11.64); 2.076(0.91); 1.235(0.69) |
| 373 | 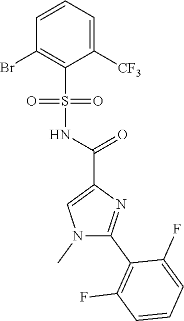 [SMILES: Cn1cc(C(=O)NS(=O)(=O)c2c(Br)cccc2C(F)(F)F)nc1-c1c(F)cccc1F] | Example 373: ¹H-NMR(400.0 MHz, d₆-DMSO): δ = 8.255(7.69); 8.245(0.52); 8.153(2.54); 8.134(2.64); 8.133(2.71); 8.064(2.3); 8.046(2.57); 7.773(0.43); 7.756(0.98); 7.752(0.98); 7.735(1.8); 7.718(1.11); 7.71(1.88); 7.689(2.83); 7.669(1.31); 7.377(3.09); 7.357(5.07); 7.337(2.64); 3.612(16); 3.001(0.6); 2.545(64.58); 2.524(0.53); 2.514(4.67); 2.51(9.77); 2.506(13.69); 2.501(10.56); 2.497(5.42); 2.077(1.24); 1.26(0.34); 1.235 (0.72) |
| 374 | 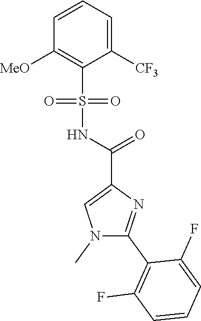 [SMILES: COc1cccc(C(F)(F)F)c1S(=O)(=O)NC(=O)c1cn(C)c(-c2c(F)cccc2F)n1] | Example 374: ¹H-NMR(400.0 MHz, d₆-DMSO): δ = 8.226(5.79); 7.834(0.88); 7.813(2.02); 7.793(1.32); 7.752(0.33); 7.734 (0.77); 7.73(0.77); 7.713(1.41); 7.696(0.82); 7.693(0.88); 7.676(0.38); 7.616 (2.32); 7.595(2.02); 7.584(2.44); 7.564(2.01); 7.366(2.43); 7.346(3.92); 7.326(2.07); 3.866(16); 3.583(12.84); 3.346(3.18); 2.671(0.33); 2.541 (20.04); 2.506(37.12); 2.502(49.25); 2.498(39); 2.074(0.41); 1.299(0.5); 1.259(0.65); 1.249(0.33); 1.235(0.83); 0(0.84) |
| 375 | 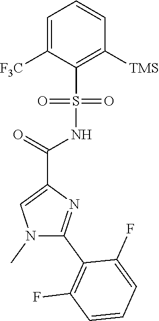 [SMILES: Cn1cc(C(=O)NS(=O)(=O)c2c(C(F)(F)F)cccc2[Si](C)(C)C)nc1-c1c(F)cccc1F] | Example 375: ¹H-NMR(400.0 MHz, d₆-DMSO): δ = 8.201(0.95); 8.141(0.75); 8.122(0.84); 7.988(0.72); 7.969(0.94); 7.855 (0.47); 7.836(0.75); 7.817(0.34); 7.734(0.39); 7.713(0.6); 7.696(0.41); 7.36(0.97); 7.34(1.71); 7.319(0.85); 3.589(5.1); 2.545(7.29); 2.543(9.66); 2.504(11.18); 2.075(0.32); 1.236(0.56); 0.4(16) |
| 376 | 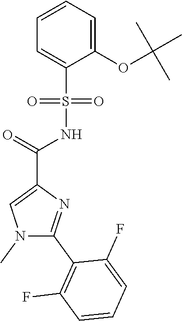 [SMILES: Cn1cc(C(=O)NS(=O)(=O)c2ccccc2OC(C)(C)C)nc1-c1c(F)cccc1F] | Example 376: ¹H-NMR(400.0 MHz, d₆-DMSO): δ = 8.182(2.1); 7.912(0.6); 7.907(0.66); 7.892(0.67); 7.888(0.67); 7.72(0.5); 7.59(0.54); 7.572(0.35); 7.568(0.34); 7.375(0.85); 7.36(1.16); 7.355(1.46); 7.339(0.89); 7.335(0.89); 7.162(0.44); 7.143(0.79); 7.124(0.39); 3.576 (4.49); 3.339(0.92); 2.542(6); 2.507(6.53); 2.503(9.09); 2.499(7.06); 1.427 (16); 1.235(0.44) |
| 377 | 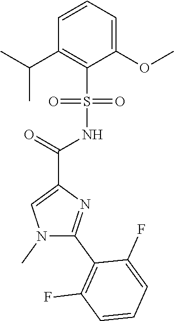 [SMILES: COc1cccc(C(C)C)c1S(=O)(=O)NC(=O)c1cn(C)c(-c2c(F)cccc2F)n1] | Example 377: ¹H-NMR(400.0 MHz, d₆-DMSO): δ = 8.218(5.36); 8.181(0.85); 7.748(0.35); 7.732(0.79); 7.727(0.72); 7.711 (1.38); 7.694(0.78); 7.689(0.83); 7.673(0.38); 7.567(1.35); 7.546(2.54); 7.526(1.47); 7.373(0.74); 7.364(2.49); 7.352(1.12); 7.344(3.77); 7.323 (2.09); 7.162(2.16); 7.144(2.13); 7.054(2.01); 7.034(1.9); 4.429(0.33); 4.412(0.86); 4.395(1.22); 4.378(0.89); 4.361(0.34); 3.783(16); 3.575(13.7); 3.323(4.14); 2.671(0.41); 2.541(0.36); 2.524(1.14); 2.506(50.19); 2.502 (68.17); 2.497(50.65); 2.329(0.4); 1.427(7.43); 1.248(13.05); 1.231(13.02); 0.008(0.89); 0(25.6); −0.008(0.92) |
| 378 | 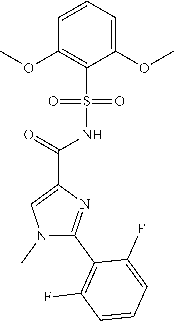 | Example 378: ¹H-NMR(400.0 MHz, d₆-DMSO): δ = 8.217(3.03); 7.736(0.37); 7.732(0.36); 7.715(0.67); 7.698(0.37); 7.694(0.4); 7.544(0.68); 7.522(1.42); 7.501(0.78); 7.369(1.18); 7.349(1.87); 7.329 (0.99); 6.808(2.94); 6.787(2.79); 3.777(16); 3.586(6.17); 3.342(5.68); 2.542 (10.69); 2.507(11.33); 2.502(15.52); 2.498(11.88); 2.074(0.55); 1.236(0.74); 0(0.33) |
| 379 | 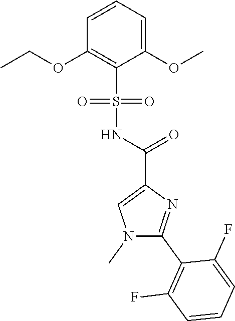 [SMILES: CCOc1cccc(OC)c1S(=O)(=O)NC(=O)c1cn(C)c(-c2c(F)cccc2F)n1] | Example 379: ¹H-NMR(400.0 MHz, d₆-DMSO): δ = 8.206(6.1); 7.738(0.73); 7.734(0.73); 7.718(1.36); 7.7(0.78); 7.697(0.84); 7.68(0.36); 7.519(1.29); 7.498(2.71); 7.477(1.48); 7.371(2.29); 7.351(3.73); 7.331(1.99); 6.799(2.48); 6.785(2.58); 6.778(2.61); 6.764(2.27); 4.089 (1.15); 4.072(3.7); 4.054(3.75); 4.037(1.18); 3.79(16); 3.584(12.64); 3.404 (0.33); 3.343(3.06); 2.543(24.68); 2.526(0.44); 2.508(9.84); 2.504(13.34); 2.5(10.46); 2.076(0.83); 1.254(4.01); 1.236(8.75); 1.219(3.87) |
| 380 | 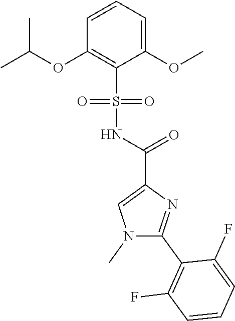 [SMILES: COc1cccc(OC(C)C)c1S(=O)(=O)NC(=O)c1cn(C)c(-c2c(F)cccc2F)n1] | Example 380: ¹H-NMR(400.0 MHz, d₆-DMSO): δ = 8.206(5.98); 7.739(0.69); 7.735(0.72); 7.718(1.33); 7.701(0.74); 7.697 (0.83); 7.68(0.34); 7.506(1.32); 7.485(2.77); 7.464(1.52); 7.372(2.28); 7.352(3.59); 7.331(1.97); 6.795(2.21); 6.773(4.31); 6.752(2.23); 4.74(0.4); 4.726(1.03); 4.71(1.4); 4.695(1.04); 4.68(0.41); 3.795(15.63); 3.583(12.18); 3.336(9.97); 2.542(20.48); 2.507(14.81); 2.503(20.69); 2.498(16.25); 2.075 (0.92); 1.259(0.41); 1.25(0.38); 1.235(1.3); 1.178(15.85); 1.163(16); 0(0.43) |
| 381 | 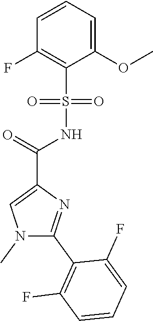 | Example 381: ¹H-NMR(400.0 MHz, d₆-DMSO): δ = 8.236(5.43); 7.747(0.37); 7.73(0.83); 7.726(0.83); 7.709(1.45); 7.692(0.81); 7.688(0.88); 7.67(0.91); 7.655(0.73); 7.649(1.29); 7.634(1.25); 7.628(0.76); 7.613(0.63); 7.362(2.54); 7.341(3.8); 7.321(2.1); 7.203(0.33); 7.076(0.39); 7.064(1.9); 7.043(1.79); 7(1.01); 6.979(1.07); 6.973(1.14); 6.951(0.98); 3.836(16); 3.821(0.87); 3.793(0.4); 3.789(0.37); 3.68(0.51); 3.665(0.4); 3.65(0.43); 3.637(0.41); 3.579(12.77); 3.509(1.14); 3.35(37.22); 2.995 (1.69); 2.676(0.91); 2.671(1.19); 2.667(0.93); 2.541(37.68); 2.506(140.26); 2.502(195.81); 2.498(150.15); 2.333(0.76); 2.329(1.03); 2.324(0.78); 2.294 (0.56); 2.074(5.28); 1.473(0.35); 1.433(0.42); 1.335(1); 1.298(1.55); 1.293 (0.9); 1.278(0.64); 1.259(2.23); 1.249(1.79); 1.235(4.86); 0.854(0.45); 0 (2.66) |
| 382 | 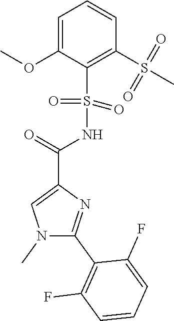 | Example 382: ¹H-NMR(400.0 MHz, d₆-DMSO): δ = 8.249(5.7); 7.897(0.45); 7.886(5.36); 7.877(3.05); 7.871(2.79); 7.851(0.41); 7.732(0.63); 7.728(0.6); 7.711(1.16); 7.694(0.86); 7.69(0.83); 7.682(1.45); 7.673(1.58); 7.667(1.12); 7.658(1.15); 7.363(1.92); 7.343(3.11); 7.322 (1.67); 3.907(14.76); 3.595(16); 3.58(11.22); 3.515(0.47); 3.478(0.51); 3.473(0.53); 3.46(0.52); 3.418(0.62); 3.402(0.6); 3.373(0.53); 3.366(0.53); 3.349(0.5); 3.331(0.47); 3.304(0.43); 2.996(0.41); 2.542(20.5); 2.512(7.22); 2.507(15.41); 2.503(21.64); 2.498(16.37); 2.494(8.06); 1.235(0.56); 0(0.57) |
| 383 | 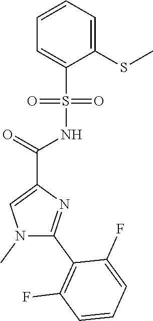 | Example 383: ¹H-NMR(400.0 MHz, d₆-DMSO): δ = 8.236(6.33); 8.055(2.09); 8.052(2.24); 8.035(2.28); 8.032(2.3); 7.742(0.38); 7.725(0.84); 7.721(0.84); 7.704(1.57); 7.687(0.88); 7.683(0.97); 7.666 (0.49); 7.66(0.86); 7.657(0.9); 7.639(1.94); 7.621(1.24); 7.618(1.24); 7.485 (2.68); 7.465(2.11); 7.393(1.42); 7.374(2.46); 7.354(3.66); 7.334(4.29); 7.313(2.25); 3.743(0.57); 3.568(16); 3.454(0.94); 3.397(0.78); 3.389(0.82); 3.297(0.5); 3.283(0.47); 2.996(0.38); 2.542(41.38); 2.507(20.31); 2.503 (28.02); 2.499(22.23); 2.493(27.5); 2.074(1.04); 1.235(0.76); 0(0.46) |
| 384 | 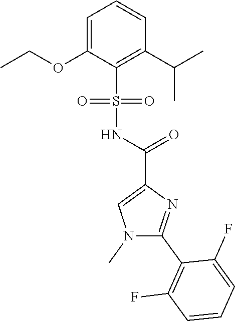 [SMILES: CCOc1cccc(C(C)C)c1S(=O)(=O)NC(=O)c1cn(C)c(-c2c(F)cccc2F)n1] | Example 384: ¹H-NMR(400.0 MHz, d₆-DMSO): δ = 8.194(6.82); 7.758(0.34); 7.741(0.81); 7.736(0.78); 7.72(1.51); 7.703(0.83); 7.699(0.93); 7.682(0.4); 7.55(1.35); 7.53(2.85); 7.51(1.65); 7.374(2.59); 7.354(4.08); 7.333(2.25); 7.327(0.55); 7.15(2.37); 7.131(2.13); 7.04(2.26); 7.02(2.08); 4.425(0.36); 4.408(0.97); 4.39(1.35); 4.374(1.01); 4.357(0.39); 4.096(1.15); 4.078(3.75); 4.061(3.81); 4.044(1.19); 3.574(13.33); 3.342 (14.92); 2.543(29.37); 2.508(21.83); 2.503(29.72); 2.499(22.55); 2.074 (0.83); 1.283(3.94); 1.266(8.42); 1.252(16); 1.235(15.42) |
| 385 | 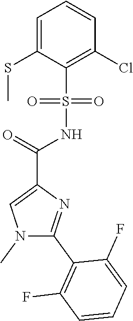 [SMILES: CSc1cccc(Cl)c1S(=O)(=O)NC(=O)c1cn(C)c(-c2c(F)cccc2F)n1] | Example 385: ¹H-NMR(400.0 MHz, d₆-DMSO): δ = 8.227(6.27); 7.745(0.39); 7.728(0.96); 7.707(1.68); 7.69(1.05); 7.669(0.53); 7.553(1.33); 7.533(3.12); 7.513(2.31); 7.419(2.83); 7.398(2.1); 7.374(3.23); 7.356(4.94); 7.336(4.66); 7.316(2.37); 4.113(0.35); 4.096(0.36); 4.09(0.37); 4.054(0.39); 3.916(0.48); 3.896(0.47); 3.858(0.47); 3.806(0.44); 3.754 (0.47); 3.675(0.34); 3.578(16); 2.542(46.65); 2.507(18.83); 2.503(24.32); 2.499(18.34); 2.464(15.93); 2.434(0.88); 2.075(0.77); 1.313(1.11); 1.236 (0.75) |
| 386 | 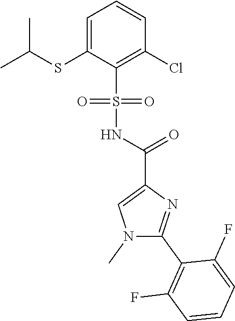 [SMILES: CC(C)Sc1cccc(Cl)c1S(=O)(=O)NC(=O)c1cn(C)c(-c2c(F)cccc2F)n1] | Example 386: ¹H-NMR(400.0 MHz, d₆-DMSO): δ = 8.226(5.09); 7.748(0.34); 7.731(0.78); 7.727(0.77); 7.71(1.46); 7.693(0.81); 7.689(0.9); 7.672(0.38); 7.592(1.35); 7.572(2.4); 7.535(1.73); 7.516(2.9); 7.496(1.4); 7.414(2.48); 7.394(1.77); 7.36(2.49); 7.34(3.97); 7.32(2.13); 3.841(0.48); 3.801(0.57); 3.789(0.58); 3.783(0.6); 3.765(0.65); 3.752(0.72); 3.728(0.77); 3.712(1.11); 3.695(1.76); 3.679(2.16); 3.663(1.82); 3.646 (1.19); 3.629(0.86); 3.576(14.31); 3.397(0.44); 2.542(46.22); 2.526(0.5); 2.508(20.69); 2.503(28.9); 2.499(22.21); 2.075(1); 1.235(0.78); 1.212 (15.86); 1.196(16) |
| 387 | 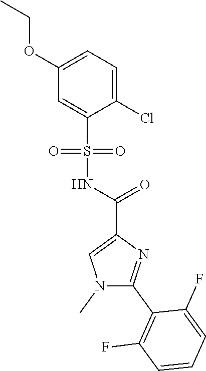 [SMILES: CCOc1ccc(Cl)c(S(=O)(=O)NC(=O)c2cn(C)c(-c3c(F)cccc3F)n2)c1] | Example 387: ¹H-NMR(400.0 MHz, d₆-DMSO): δ = 8.243(5.47); 7.747(0.38); 7.73(0.85); 7.726(0.84); 7.709(1.57); 7.692(0.89); 7.688(0.98); 7.671(0.41); 7.587(3.81); 7.579(4.05); 7.553(3.04); 7.531 (3.55); 7.358(2.63); 7.337(4.35); 7.317(2.27); 7.272(1.84); 7.264(1.8); 7.25 (1.61); 7.242(1.54); 4.144(1.53); 4.126(4.57); 4.109 (4.62); 4.092(1.62); 3.753(1.18); 3.622(1.58); 3.578(16); 3.399(0.55); 3.382(0.44); 2.672(0.36); 2.542(3.73); 2.507(39.24); 2.502(53.32); 2.498(41.22); 1.377(4.73); 1.359 (9.88); 1.342(4.59); 1.259(0.35); 1.25(0.33); 1.235(0.67); 0(0.74) |
| 388 | 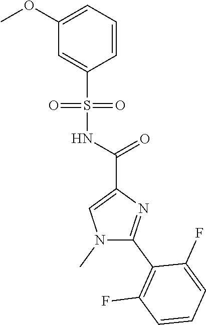 [SMILES: COc1cccc(S(=O)(=O)NC(=O)c2cn(C)c(-c3c(F)cccc3F)n2)c1] | Example 388: ¹H-NMR(400.0 MHz, d₆-DMSO): δ = 8.171(5.37); 7.723(0.77); 7.706(1.34); 7.685(0.87); 7.668(0.35); 7.573(0.6); 7.558(4.83); 7.539(2.41); 7.511(2.69); 7.355(2.26); 7.335(3.71); 7.314 (1.97); 7.282(1.51); 7.276(1.03); 7.269(0.96); 7.264(1.41); 7.259(0.88); 3.828(16); 3.646(0.42); 3.561(12.55); 3.494(0.59); 3.491(0.59); 3.463 (0.61); 3.437(0.61); 3.42(0.62); 3.381(0.64); 3.322(0.51); 2.542(24.28); 2.503(28.73); 2.075(0.93); 1.235(0.7); 0(0.43) |
| 389 | 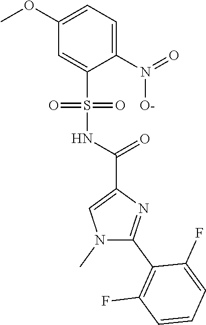 | Example 389: ¹H-NMR(400.0 MHz, d₆-DMSO): δ = 8.274(5.25); 8.012(2.96); 7.99(3.2); 7.763(0.68); 7.758(0.65); 7.742(1.25); 7.724(0.67); 7.72(0.76); 7.704(0.33); 7.659(3.1); 7.652(3.28); 7.41(1.65); 7.404(1.6); 7.385(2.67); 7.382(2.08); 7.364(3.52); 7.344(1.87); 4.242(0.32); 4.214(0.33); 4.201(0.33); 4.178(0.33); 4.156(0.34); 4.105(0.41); 4.054 (0.33); 3.924(16); 3.618(11.16); 2.543(12.68); 2.512(6.77); 2.508(14.34); 2.503(20.22); 2.499(15.44); 2.495(7.7); 1.235(0.55); 0(0.47) |
| 390 | 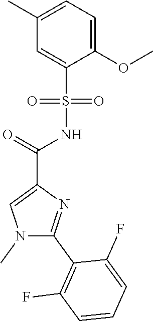 | Example 390: ¹H-NMR(400.0 MHz, d₆-DMSO): δ = 8.215(5.88); 7.744(0.32); 7.728(0.74); 7.724(0.77); 7.707(1.48); 7.695 (2.87); 7.69(3.6); 7.669(0.37); 7.48(1.27); 7.476(1.32); 7.459(1.44); 7.454 (1.46); 7.359(2.34); 7.339(3.85); 7.319(2.03); 7.134(2.76); 7.113(2.47); 3.801(16); 3.571(12.78); 3.338(3.29); 2.542(29.85); 2.506(23.49); 2.502 (32.15); 2.499(26.14); 2.329(12.72); 2.074(0.76); 1.235(0.89); 0(0.52) |
| 391 | 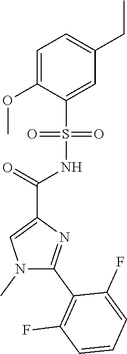 [SMILES: CCc1ccc(OC)c(S(=O)(=O)NC(=O)c2cn(C)c(-c3c(F)cccc3F)n2)c1] | Example 391: ¹H-NMR(400.0 MHz, d₆-DMSO): δ = 8.219(6.01); 7.744(0.32); 7.727(0.73); 7.723(0.77); 7.711(3.08); 7.706 (4.26); 7.689(0.78); 7.685(0.88); 7.668(0.36); 7.519(1.3); 7.513(1.3); 7.498 (1.48); 7.492(1.46); 7.366(0.45); 7.359(2.4); 7.338(3.69); 7.318(2.06); 7.311(0.45); 7.158(2.84); 7.137(2.56); 3.808(16); 3.571(11.99); 3.337 (1.64); 2.671(1.11); 2.652(3.02); 2.633(3.11); 2.614(1.06); 2.542(1.45); 2.511(8.47); 2.507(17.54); 2.502(24.55); 2.498(19.01); 2.494(9.72); 1.235 (0.68); 1.205(4.16); 1.186(8.78); 1.168(4.02); 0(0.45) |
| 392 | 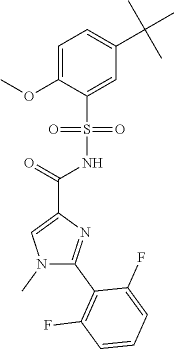 [SMILES: COc1ccc(C(C)(C)C)cc1S(=O)(=O)NC(=O)c1cn(C)c(-c2c(F)cccc2F)n1] | Example 392: ¹H-NMR(400.0 MHz, d₆-DMSO): δ = 8.223(2.04); 7.855(1.04); 7.849(1.12); 7.707(0.87); 7.701(0.73); 7.686 (0.85); 7.679(0.63); 7.358(0.83); 7.338(1.3); 7.318(0.71); 7.169(0.98); 7.147(0.91); 3.818(5.25); 3.572(4.13); 3.34(0.75); 2.542(13.72); 2.507 (6.44); 2.503(8.58); 2.498(6.53); 1.3(16) |
| 393 | 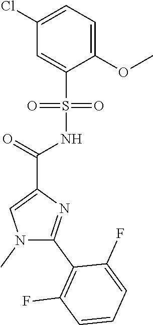 | Example 393: ¹H-NMR(400.0 MHz, d₆-DMSO): δ = 8.233(5.4); 7.832(3.31); 7.825(3.84); 7.755(1.54); 7.748(1.35); 7.733(1.75); 7.726(2.04); 7.706(1.28); 7.69(0.7); 7.685(0.77); 7.668(0.33); 7.358(2.16); 7.338(3.46); 7.317(1.85); 7.31(0.43); 7.296(2.86); 7.273(2.59); 3.862(16); 3.75(0.34); 3.676(0.44); 3.575(12.25); 3.518(0.49); 3.395(0.45); 3.355 (0.35); 2.543(1.59); 2.508(14.28); 2.503(19.85); 2.499(15.05); 1.235(0.58) |
| 394 | 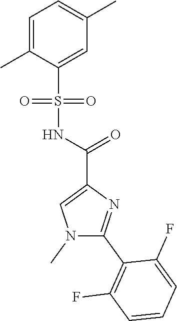 [SMILES: Cc1ccc(C)c(S(=O)(=O)NC(=O)c2cn(C)c(-c3c(F)cccc3F)n2)c1] | Example 394: ¹H-NMR(400.0 MHz, d₆-DMSO): δ = 8.17(4.65); 7.828(3.19); 7.74(0.41); 7.724(0.9); 7.719(0.84); 7.702(1.72); 7.686(0.9); 7.681(1.03); 7.664(0.45); 7.388(1.25); 7.368(1.81); 7.359(0.66); 7.352(3.03); 7.331(4.44); 7.311(2.56); 7.304(0.49); 7.278(2.8); 7.258(2.01); 3.561(16); 3.355(16.17); 2.995(0.68); 2.711(0.57); 2.676(0.64); 2.671 (0.84); 2.667(0.65); 2.541(136.28); 2.524(2.07); 2.52(2.94); 2.511(44.16); 2.506(95.11); 2.502(134.13); 2.497(100.25); 2.493(47.87); 2.363(13.18); 2.333(0.6); 2.329(0.82); 2.324(0.61); 0(2.61) |
| 395 | 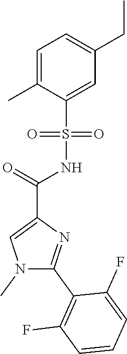 [SMILES: CCc1ccc(C)c(S(=O)(=O)NC(=O)c2cn(C)c(-c3c(F)cccc3F)n2)c1] | Example 395: ¹H-NMR(400.0 MHz, d₆-DMSO): δ = 8.179(5.2); 8.164(0.48); 7.855(3.02); 7.851(3.2); 7.74(0.43); 7.724(0.96); 7.719(0.86); 7.706(0.7); 7.702(1.74); 7.686(0.91); 7.681(1.04); 7.664(0.47); 7.429(1.32); 7.425(1.31); 7.41(1.78); 7.405(1.74); 7.359(0.56); 7.352(3.13); 7.332(4.79); 7.311(2.97); 7.304(3.2); 7.284(1.98); 3.561(16); 3.356(6.13); 3.126(0.46); 3.003(0.47); 2.995(0.51); 2.985(0.46); 2.711(0.55); 2.701 (1.12); 2.682(3.44); 2.671(0.97); 2.663(3.6); 2.644(1.2); 2.556(14.06); 2.541(109.36); 2.524(1.51); 2.52(2.1); 2.511(32.32); 2.506(69.08); 2.502 (96.98); 2.497(72.33); 2.493(34.65); 2.366(1.34); 2.333(0.42); 2.329(0.58); 2.324(0.42); 1.216(5.37); 1.197(11.68); 1.178(5.21); 1.146(0.38); 1.128 (0.82); 1.109(0.37); 0(2.01) |
| 396 | 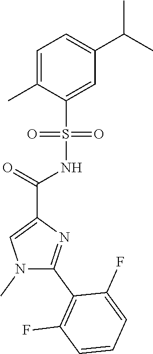 [SMILES: Cc1ccc(C(C)C)cc1S(=O)(=O)NC(=O)c1cn(C)c(-c2c(F)cccc2F)n1] | Example 396: ¹H-NMR(400.0 MHz, d₆-DMSO): δ = 8.187(4.3); 7.883(2.77); 7.879(2.98); 7.74(0.36); 7.723(0.79); 7.719(0.77); 7.702(1.42); 7.685(0.79); 7.681(0.9); 7.664(0.37); 7.467(1.34); 7.463(1.41); 7.448(1.6); 7.444(1.61); 7.351(2.46); 7.331(3.77); 7.312(3.66); 7.294(1.87); 3.561(12.95); 3.36(5.23); 3.159(0.6); 3.144(0.54); 3.005(0.56); 2.995(0.55); 2.988(1.08); 2.971(1.39); 2.954(1.08); 2.936(0.52); 2.711(0.4); 2.676(0.39); 2.671(0.5); 2.557(12.73); 2.541(77.69); 2.506(52.86); 2.502(72.17); 2.498 (55.58); 2.363(0.61); 2.333(0.37); 2.329(0.47); 2.325(0.36); 1.229(16); 1.212(15.79); 1.125(0.51); 1.108(0.5); 0(0.94) |
| 397 | 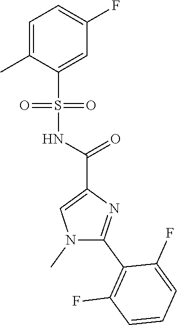 [SMILES: Cc1ccc(F)cc1S(=O)(=O)NC(=O)c1cn(C)c(-c2c(F)cccc2F)n1] | Example 397: ¹H-NMR(400.0 MHz, d₆-DMSO): δ = 8.19(5.5); 7.773(1.37); 7.768(1.47); 7.747(1.68); 7.731(0.86); 7.727(0.8); 7.71(1.6); 7.693(0.81); 7.689(0.95); 7.672(0.41); 7.474(1.55); 7.468(1.87); 7.464(2.28); 7.455(1.91); 7.449(3.49); 7.364(0.49); 7.358(2.8); 7.337(4.1); 7.317(2.36); 7.31(0.45); 3.743(0.65); 3.569(16); 3.477(3.65); 3.196(0.37); 3.175(0.33); 2.995(0.39); 2.712(0.45); 2.676(0.38); 2.672(0.49); 2.667 (0.38); 2.57(12.17); 2.542(99.31); 2.525(1.18); 2.511(23.53); 2.507(50.44); 2.502(70.98); 2.498(52.89); 2.493(25.09); 2.368(0.37); 2.329(0.42); 0(0.81) |
| 398 | 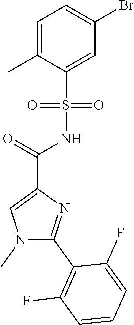 [SMILES: Cc1ccc(Br)cc1S(=O)(=O)NC(=O)c1cn(C)c(-c2c(F)cccc2F)n1] | Example 398: ¹H-NMR(400.0 MHz, d₆-DMSO): δ = 8.189(4.27); 8.103(3.72); 8.098(3.89); 7.79(1.68); 7.785(1.63); 7.77(1.81); 7.765(1.8); 7.748(0.45); 7.727(0.98); 7.711(1.7); 7.689(1.1); 7.673(0.43); 7.377(2.7); 7.358(5.1); 7.338(4.62); 7.317(2.55); 3.669(0.34); 3.569(16); 3.456(0.7); 3.444(0.76); 3.388(0.83); 3.36(0.75); 3.267(0.61); 3.251(0.58); 3.224(0.49); 3.186(0.44); 3.126(0.33); 2.67(1.04); 2.558(16.25); 2.505 (145.3); 2.501(192.4); 2.497(147.1); 2.328(1.12); 2.074(0.64); 0.146(0.57); 0(132.2); −0.15(0.57) |
| 399 | 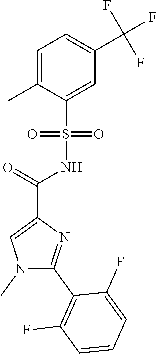 [SMILES: Cc1ccc(C(F)(F)F)cc1S(=O)(=O)NC(=O)c1cn(C)c(-c2c(F)cccc2F)n1] | Example 399: ¹H-NMR(400.0 MHz, d₆-DMSO): δ = 8.27(2.75); 8.193(4.28); 7.969(1.23); 7.949(1.41); 7.752(0.38); 7.736(0.8); 7.731(0.76); 7.714(1.47); 7.698(0.79); 7.693(0.92); 7.676(0.49); 7.666 (1.91); 7.646(1.69); 7.36(2.59); 7.34(3.98); 7.32(2.22); 7.313(0.47); 3.81 (0.43); 3.745(0.68); 3.57(16); 3.464(7.02); 3.275(0.53); 2.995(0.37); 2.711 (0.58); 2.694(10.58); 2.676(0.62); 2.671(0.7); 2.667(0.48); 2.542(106.47); 2.525(1.39); 2.511(29.79); 2.507(63.91); 2.502(90.07); 2.498(67.85); 2.494 (32.95); 2.368(0.42); 2.333(0.4); 2.329(0.54); 2.325(0.4); 0(1.8) |
| 400 | 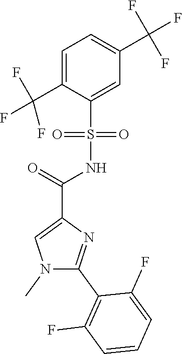 [SMILES: Cn1cc(C(=O)NS(=O)(=O)c2cc(C(F)(F)F)ccc2C(F)(F)F)nc1-c1c(F)cccc1F] | Example 400: ¹H-NMR(400.0 MHz, d₆-DMSO): δ = 8.604(2.84); 8.231(4.9); 8.22(2.07); 8.182(2.66); 8.162(1.33); 7.806(0.36); 7.79(0.8); 7.785(0.75); 7.768(1.49); 7.752(0.78); 7.747(0.9); 7.73(0.39); 7.401(2.66); 7.381(4.41); 7.36(2.26); 3.811(0.36); 3.635(16); 3.474(12.16); 2.995(0.56); 2.676(0.5); 2.672(0.67); 2.667(0.51); 2.542(63.63); 2.525 (1.59); 2.52(2.34); 2.511(37.18); 2.507(80.05); 2.502(112.91); 2.498 (84.85); 2.494(40.93); 2.334(0.46); 2.329(0.66); 2.325(0.48); 0(2.5) |
| 401 | 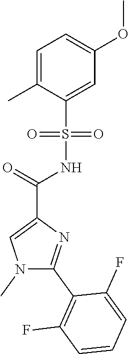 [SMILES: COc1ccc(C)c(S(=O)(=O)NC(=O)c2cn(C)c(-c3c(F)cccc3F)n2)c1] | Example 401: ¹H-NMR(400.0 MHz, d₆-DMSO): δ = 8.18(3.89); 7.726(0.59); 7.721(0.56); 7.705(1.1); 7.688(0.58); 7.684(0.66); 7.531(2.38); 7.524(2.5); 7.361(0.36); 7.354(1.97); 7.334(2.85); 7.314(2.41); 7.306(0.41); 7.295(1.91); 7.173(1.27); 7.166(1.24); 7.152(0.98); 7.145 (0.96); 3.804(16); 3.621(0.5); 3.564(10.3); 3.52(0.95); 3.406(2.23); 3.385 (2.24); 2.672(0.33); 2.542(52.67); 2.511(17.66); 2.507(33.14); 2.502 (46.26); 2.498(34.77); 2.493(16.88); 0(0.64) |
| 402 | 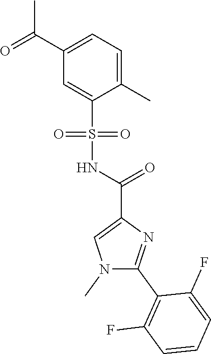 [SMILES: CC(=O)c1ccc(C)c(S(=O)(=O)NC(=O)c2cn(C)c(-c3c(F)cccc3F)n2)c1] | Example 402: ¹H-NMR(400.0 MHz, d₆-DMSO): δ = 8.54(2.6); 8.535(2.79); 8.169(2.57); 8.156(1.29); 8.152(1.22); 8.136(1.24); 8.132(1.26); 7.728(0.65); 7.723(0.63); 7.707(1.21); 7.69(0.63); 7.686(0.74); 7.572(1.73); 7.552(1.6); 7.355(2.13); 7.334(3.2); 7.314(1.82); 3.736(0.4); 3.562(12.06); 3.409(7.82); 3.09(0.35); 2.995(0.54); 2.711(0.48); 2.68 (11.32); 2.621(16); 2.541(102.15); 2.524(1.55); 2.511(35.27); 2.506(76.03); 2.502(107.61); 2.497(81.82); 2.493(40.33); 2.367(0.46); 2.333(0.49); 2.329 (0.66); 2.324(0.5); 0(1.31) |
| 403 | 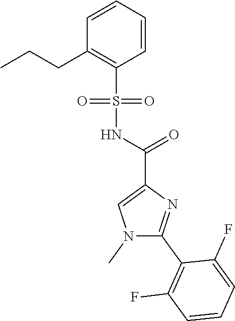 [SMILES: CCCc1ccccc1S(=O)(=O)NC(=O)c1cn(C)c(-c2c(F)cccc2F)n1] | Example 403: ¹H-NMR(400.0 MHz, d₆-DMSO): δ = 8.159(6.5); 8.031(1.87); 8.028(2.23); 8.01(2.27); 8.007(2.45); 7.741(0.4); 7.725(0.9); 7.72(0.86); 7.703(1.7); 7.687(0.88); 7.682(1.04); 7.666(0.44); 7.616(0.84); 7.612(0.92); 7.597(1.98); 7.594(1.95); 7.578(1.32); 7.575 (1.33); 7.434(2.97); 7.415(3.12); 7.401(1.16); 7.36(0.49); 7.353(2.97); 7.332 (4.38); 7.312(2.55); 7.305(0.5); 3.737(0.33); 3.561(16); 3.43(1.29); 3.424 (1.3); 3.382(1.32); 2.999(2.25); 2.979(2.5); 2.973(1.81); 2.959(2.41); 2.712 (0.32); 2.542(74); 2.525(0.6); 2.52(0.73); 2.511(13.41); 2.507(29.38); 2.502 (41.89); 2.498(31.76); 2.494(15.67); 2.368(0.35); 1.586(1.14); 1.567(1.84); 1.547(1.93); 1.528(1.27); 1.51(0.34); 0.932(4.64); 0.914(9.8); 0.896(4.22); 0(0.53) |
| 404 | 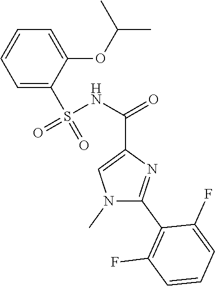 [SMILES: CC(C)Oc1ccccc1S(=O)(=O)NC(=O)c1cn(C)c(-c2c(F)cccc2F)n1] | Example 404: ¹H-NMR(400.0 MHz, d₆-DMSO): δ = 8.204(5.01); 7.899(1.65); 7.895(1.84); 7.88(1.82); 7.875(1.89); 7.754(0.32); 7.737(0.72); 7.732(0.67); 7.716(1.34); 7.699(0.7); 7.694(0.83); 7.678(0.36); 7.65(0.72); 7.646(0.75); 7.628(1.37); 7.61(0.9); 7.606(0.88); 7.375(0.42); 7.368(2.35); 7.348(3.47); 7.327(1.98); 7.32(0.39); 7.239(1.99); 7.219(1.78); 7.12(1.16); 7.102(2.08); 7.082(1.02); 4.821(0.37); 4.806(0.96); 4.791(1.31); 4.776(0.98); 4.761(0.38); 3.571(11.96); 3.336(10.07); 2.542(27.49); 2.525 (0.6); 2.511(13.66); 2.507(29.17); 2.502(41.01); 2.498(30.78); 2.494 (14.84); 1.17(15.93); 1.155(16); 0(0.46) |
| 405 | 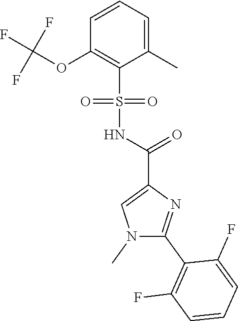 [SMILES: Cc1cccc(OC(F)(F)F)c1S(=O)(=O)NC(=O)c1cn(C)c(-c2c(F)cccc2F)n1] | Example 405: ¹H-NMR(400.0 MHz, d₆-DMSO): δ = 8.315(0.78); 8.194(2.18); 7.744(0.38); 7.728(0.92); 7.724(0.88); 7.707 (1.59); 7.689(0.92); 7.686(1); 7.669(1.26); 7.649(1.89); 7.629(1.18); 7.44 (1.93); 7.42(1.71); 7.401(1.4); 7.379(1.2); 7.362(0.67); 7.355(2.88); 7.335 (4.43); 7.314(2.39); 5.755(2.8); 3.631(0.34); 3.614(0.37); 3.571(14.81); 3.526(0.45); 3.51(0.52); 3.483(0.56); 3.458(0.69); 3.454(0.7); 3.354(1.26); 3.348(1.26); 3.34(1.25); 3.321(1.19); 3.221(0.64); 3.212(0.61); 3.134(0.35); 2.718(16); 2.675(1.66); 2.67(2.2); 2.666(1.65); 2.541(1.89); 2.524(5.66); 2.506(260.04); 2.501(355.94); 2.497(264.85); 2.333(1.47); 2.328(2); 2.324 (1.5); 0.146(0.69); 0.008(5.37); 0(156.17); −0.008(5.74); −0.15(0.69) |
| 406 | 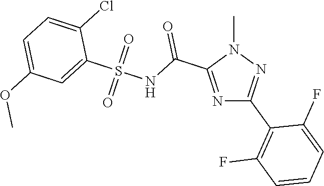 [SMILES: COc1ccc(Cl)c(S(=O)(=O)NC(=O)c2nc(-c3c(F)cccc3F)nn2C)c1] | Example 406: ¹H-NMR(400.0 MHz, d₆-DMSO): δ = 7.643(0.63); 7.633(2.52); 7.625(2.91); 7.606(0.59); 7.601(0.63); 7.561 (2.42); 7.539(2.83); 7.297(1.78); 7.276(3.02); 7.264(1.67); 7.256(2.75); 7.242(1.3); 7.235(1.17); 4.098(14.35); 4.033(0.63); 3.916(0.77); 3.853(16); 3.817(1.02); 3.694(1.38); 3.688(1.39); 3.67(1.37); 2.542(26.87); 2.525 (0.54); 2.512(11.27); 2.508(23.68); 2.503(33.13); 2.499(25.12); 2.494 (12.43); 0(1.79) |
| 407 | 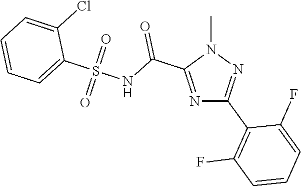 [SMILES: Cn1nc(-c2c(F)cccc2F)nc1C(=O)NS(=O)(=O)c1ccccc1Cl] | Example 407: ¹H-NMR(400.0 MHz, d₆-DMSO): δ = 8.183(1.35); 8.18(1.45); 8.163(1.44); 8.161(1.5); 7.697(0.33); 7.681(1.13); 7.677(1.11); 7.661(3.81); 7.656(2.71); 7.645(0.75); 7.641(1.31); 7.629 (0.49); 7.624(1.22); 7.61(1.43); 7.605(1.32); 7.593(0.91); 7.59(1.29); 7.586 (1.11); 7.574(0.67); 7.569(0.58); 7.307(0.34); 7.299(2.07); 7.278(3.4); 7.258(1.73); 4.089(16); 4.004(0.36); 3.955(0.55); 3.948(0.55); 3.908(0.76); 3.805(1.02); 3.726(1.39); 3.613(2.56); 2.542(35.59); 2.525(0.59); 2.512 (12.5); 2.508(26.13); 2.503(36.35); 2.499(27.36); 2.494(13.33); 0(1.54) |
| 408 | 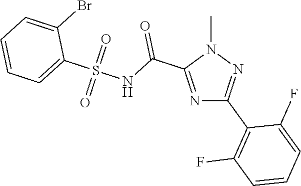 [SMILES: Cn1nc(-c2c(F)cccc2F)nc1C(=O)NS(=O)(=O)c1ccccc1Br] | Example 408: ¹H-NMR(400.0 MHz, d₆-DMSO): δ = 8.21(1.31); 8.206(1.39); 8.191(1.43); 8.187(1.44); 7.839(1.5); 7.819(1.72); 7.66(0.35); 7.646(0.84); 7.644(0.9); 7.638(0.74); 7.628(1.94); 7.622(1.48); 7.612(1.25); 7.608(1.37); 7.601(0.84); 7.585(1.23); 7.566(1.21); 7.563 (1.21); 7.547(0.49); 7.305(0.36); 7.297(2.28); 7.277(3.72); 7.256(1.91); 7.248(0.33); 4.1(16); 3.956(0.66); 3.918(0.83); 3.794(1.12); 3.616(2.28); 2.542(29.09); 2.525(0.65); 2.512(13.99); 2.507(29.24); 2.503(40.73); 2.499 (30.89); 1.235(0.42); 0(2.07) |
| 409 | 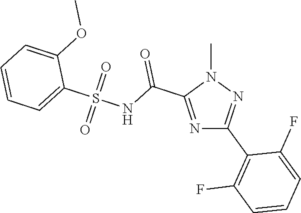 [SMILES: COc1ccccc1S(=O)(=O)NC(=O)c1nc(-c2c(F)cccc2F)nn1C] | Example 409: ¹H-NMR(400.0 MHz, d₆-DMSO): δ = 7.941(1.49); 7.937(1.65); 7.922(1.63); 7.918(1.69); 7.713(0.71); 7.708 (0.73); 7.691(1.32); 7.673(0.97); 7.669(0.98); 7.655(0.67); 7.65(0.63); 7.634 (1.29); 7.617(0.66); 7.613(0.77); 7.596(0.34); 7.32(0.39); 7.312(2.27); 7.291 (3.79); 7.27(2.36); 7.268(2.34); 7.245(1.81); 7.178(1.05); 7.16(1.92); 7.14 (0.94); 4.072(16); 3.874(15.32); 3.69(0.41); 3.409(2.68); 2.542(3.72); 2.525 (0.67); 2.511(15.41); 2.507(32.35); 2.502(45.32); 2.498(34.46); 2.494(17.1); 0(2.47) |
| 410 | 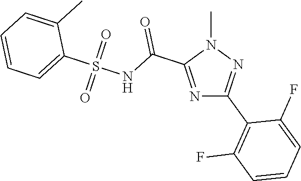 [SMILES: Cc1ccccc1S(=O)(=O)NC(=O)c1nc(-c2c(F)cccc2F)nn1C] | Example 410: ¹H-NMR(400.0 MHz, d₆-DMSO): δ = 8.084(1.51); 8.064(1.6); 7.676(0.32); 7.66(0.69); 7.655(0.65); 7.643(0.48); 7.638(1.32); 7.634(0.55); 7.623(1.25); 7.617(0.9); 7.607(1.54); 7.604(1.67); 7.589(1.01); 7.586(1.02); 7.487(0.94); 7.468(1.5); 7.449(0.72); 7.437(1.67); 7.418(1.37); 7.319(0.37); 7.311(2.25); 7.29(3.72); 7.27(1.91); 7.262(0.36); 4.074(16); 2.646(10.62); 2.546(12.28); 2.515(3.55); 2.511(7.58); 2.506 (10.67); 2.502(8.19); 2.498(4.17); 0(0.94) |
| 411 | 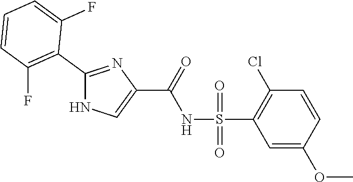 [SMILES: COc1ccc(Cl)c(S(=O)(=O)NC(=O)c2c[nH]c(-c3c(F)cccc3F)n2)c1] | Example 411: ¹H-NMR(400.0 MHz, d₆-DMSO): δ = 8.221(1.65); 8.011(0.55); 7.633(0.68); 7.625(2.82); 7.617(3); 7.612(1.21); 7.595(0.59); 7.59(0.64); 7.573(2.26); 7.551(2.39); 7.314(0.57); 7.304(1.86); 7.29(2.01); 7.283(3.89); 7.268(1.48); 7.262(1.9); 4.192(0.82); 3.856(16); 3.75(0.82); 3.732(0.91); 3.715(0.92); 3.698(0.89); 3.671(0.97); 3.612(2.41); 2.671(0.37); 2.51(23.36); 2.506(46.92); 2.502(61.52); 2.497(44.26); 2.493 (21.29); 2.328(0.39); 1.284(1.43); 1.267(1.44); 0(0.78) |
| 412 | 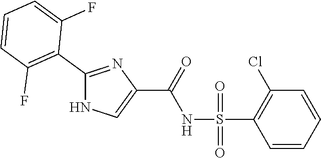 [SMILES: O=C(NS(=O)(=O)c1ccccc1Cl)c1c[nH]c(-c2c(F)cccc2F)n1] | Example 412: ¹H-NMR(601.6 MHz, CDCl3): δ = 9.994(0.58); 8.376(4.23); 8.374(4.51); 8.363(4.3); 8.361(4.48); 7.806(16); 7.556(1.62); 7.553(1.71); 7.542(3.88); 7.541(3.98); 7.53(4.37); 7.528(4.22); 7.502(8.43); 7.496(4.95); 7.494(3.03); 7.49(4.47); 7.488(4.08); 7.483(5.5); 7.471(2.49); 7.469(2.23); 7.436(1); 7.431(0.6); 7.425(2.15); 7.422(2.14); 7.415(1.52); 7.411(4.15); 7.408(1.56); 7.401(2.21); 7.397(2.36); 7.387(1.1); 7.26(71.38); 7.107(6.77); 7.092(10.72); 7.077(5.94); 3.889(0.41); 2.819 (1.65); 1.605(0.58); 1.468(0.44); 1.457(0.42); 1.254(0.65); 1.068(0.59); 0.096(0.45); 0.069(3.16); 0.005(5.04); 0(106.03); −0.006(3.91); −0.1(0.45) |
| 413 | 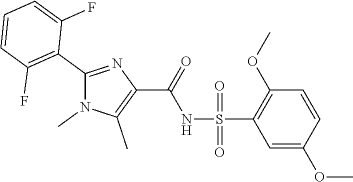 [SMILES: COc1ccc(OC)c(S(=O)(=O)NC(=O)c2nc(-c3c(F)cccc3F)n(C)c2C)c1] | Example 413: ¹H-NMR(400.0 MHz, d₆-DMSO): δ = 7.74(0.55); 7.735(0.55); 7.719(1.05); 7.702(0.57); 7.698(0.66); 7.376 (3.04); 7.369(3.15); 7.354(2.86); 7.333(1.61); 7.275(0.88); 7.267(0.75); 7.252(1.55); 7.244(1.47); 7.198(2.63); 7.175(1.45); 3.793(16); 3.761(13.8); 3.415(9.09); 2.524(0.41); 2.51(11.63); 2.506(24.5); 2.502(32.87); 2.497 (24.23); 2.493(12.14); 2.46(12.88); 0.008(0.69); 0(21.65); −0.008(0.91) |
| 414 | 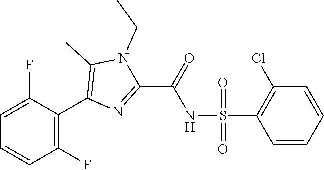 [SMILES: CCn1c(C(=O)NS(=O)(=O)c2ccccc2Cl)nc(-c2c(F)cccc2F)c1C] | Example 414: ¹H-NMR(400.0 MHz, d₆-DMSO): δ = 8.104(2.39); 8.102(2.52); 8.084(3.24); 7.693(0.63); 7.688(0.58); 7.675 (0.48); 7.658(0.99); 7.654(0.97); 7.637(1.87); 7.62(1.02); 7.616(1.19); 7.6 (0.55); 7.577(0.36); 7.558(1.4); 7.553(2); 7.548(3.06); 7.54(7.34); 7.53 (1.14); 7.522(2.13); 7.515(1.45); 7.508(1.08); 7.503(1.89); 7.495(1.19); 7.49(1.11); 7.481(0.82); 7.327(0.55); 7.32(3.29); 7.3(5.26); 7.279(2.8); 7.271(0.57); 4.548(1.01); 4.53(3.33); 4.512(3.38); 4.494(1.05); 2.524(0.76); 2.506(42.96); 2.502(57.03); 2.498(41.54); 2.329(0.34); 2.266(0.42); 2.248 (0.82); 2.222(16); 2.075(1.57); 1.397(0.38); 1.317(4.03); 1.299(9.16); 1.282 (3.98); 1.165(0.35); 1.147(0.35); 0.816(0.73); 0.798(1.42); 0.779(0.6); 0.008 (0.85); 0(24); −0.008(0.96) |
| 415 | 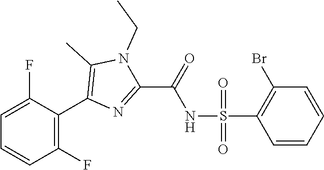 [SMILES: CCn1c(C(=O)NS(=O)(=O)c2ccccc2Br)nc(-c2c(F)cccc2F)c1C] | Example 415: ¹H-NMR(400.0 MHz, d₆-DMSO): δ = 12.435(0.5); 8.131(2.63); 8.128(2.68); 8.112(2.96); 8.108(2.7); 7.872(0.4); 7.868(0.4); 7.853(0.49); 7.849(0.48); 7.734(2.66); 7.714(3.15); 7.675(0.42); 7.658(1.05); 7.654(1.08); 7.637(2.23); 7.615(1.6); 7.599(0.57); 7.591(0.4); 7.565(1.25); 7.546(2.78); 7.527(1.75); 7.464(1.56); 7.46(1.61); 7.445(2.28); 7.441(2.23); 7.426(1.01); 7.422(0.97); 7.319(3.22); 7.299(5.25); 7.279 (2.72); 4.567(1.04); 4.549(3.35); 4.531(3.38); 4.514(1.09); 2.675(0.68); 2.67 (1); 2.666(0.69); 2.524(1.39); 2.506(149.05); 2.501(195.78); 2.497(142.44); 2.413(0.37); 2.385(0.34); 2.332(0.97); 2.328(1.26); 2.324(0.99); 2.271 (0.72); 2.253(1.38); 2.225(16); 2.074(1.97); 1.422(0.45); 1.404(0.61); 1.386 (0.49); 1.328(3.96); 1.311(9.07); 1.293(4.06); 1.191(0.35); 1.172(0.58); |
| | | 1.152(0.59); 1.135(0.37); 0.82(1.16); 0.801(2.26); 0.783(0.98); 0.008(1.77); |
| | | 0(73.27); −0.008(3.47); −0.15(0.37) |
| 416 | 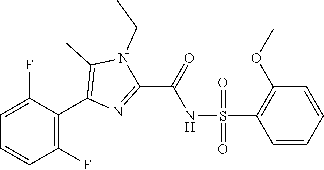 [SMILES: CCn1c(C(=O)NS(=O)(=O)c2ccccc2OC)nc(-c2c(F)cccc2F)c1C] | Example 416: ¹H-NMR(400.0 MHz, d₆-DMSO): δ = 7.906(1.46); 7.902(1.64); 7.886(1.62); 7.882(1.72); 7.676(0.63); 7.672 (0.65); 7.654(1.29); 7.637(0.79); 7.633(0.79); 7.579(0.6); 7.575(0.61); 7.558 (1.19); 7.542(0.64); 7.537(0.76); 7.521(0.33); 7.266(1.94); 7.246(3.07); 7.232(2.34); 7.226(1.91); 7.211(1.96); 7.16(1.07); 7.141(1.94); 7.122(0.99); 4.324(0.65); 4.307(2.11); 4.289(2.18); 4.271(0.7); 3.823(16); 2.674(0.4); 2.67(0.56); 2.666(0.42); 2.524(1.04); 2.51(35.35); 2.506(74.23); 2.501 (100.11); 2.497(72.89); 2.492(35.94); 2.332(0.48); 2.328(0.62); 2.324(0.47); 2.173(9.94); 1.229(2.62); 1.211(6.04); 1.193(2.59); 0.008(1.28); 0(41.85); −0.008(1.6) |
| 417 | 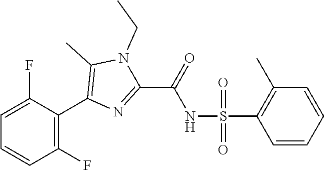 [SMILES: CCn1c(C(=O)NS(=O)(=O)c2ccccc2C)nc(-c2c(F)cccc2F)c1C] | Example 417: ¹H-NMR(400.0 MHz, d₆-DMSO): δ = 8.011(2.11); 7.993(2.2); 7.619(0.36); 7.602(0.86); 7.598(0.87); 7.582(1.62); 7.564(0.93); 7.56(1.02); 7.544(0.46); 7.526(0.82); 7.51(1.96); 7.491(1.35); 7.411(1.33); 7.392(2.05); 7.374(0.94); 7.356(2.3); 7.337(1.83); 7.278(2.72); 7.258(4.23); 7.237(2.35); 7.228(0.46); 4.384(0.92); 4.366(2.86); 4.348 (2.87); 4.33(0.98); 2.675(0.67); 2.671(0.92); 2.666(0.7); 2.602(16); 2.506 (127.82); 2.502(167.74); 2.497(124.74); 2.441(0.5); 2.333(0.81); 2.328 (1.06); 2.18(13.78); 2.074(0.94); 1.259(3.53); 1.242(8); 1.224(3.56); 0.008 (2.37); 0(59.92) |
| 418 | 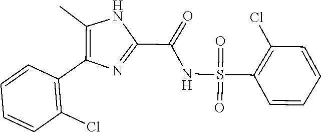 [SMILES: Cc1[nH]c(C(=O)NS(=O)(=O)c2ccccc2Cl)nc1-c1ccccc1Cl] | Example 418: ¹H-NMR(601.6 MHz, CDCl3): δ = 8.3(2.57); 8.288(2.45); 7.563(1.13); 7.552(2.47); 7.54(1.98); 7.518(3.53); 7.503(3.32); 7.496(2.5); 7.487(2.06); 7.477(1.84); 7.464(2.58); 7.452(1.14); 7.398(2.56); 7.391(2.38); 7.371(5.34); 7.365(4.74); 7.26(15.27); 5.299 (1.53); 2.285(16); 1.257(0.45); 0(23.84) |
| 419 | 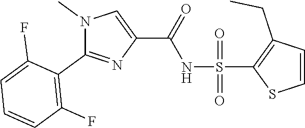 [SMILES: CCc1ccsc1S(=O)(=O)NC(=O)c1cn(C)c(-c2c(F)cccc2F)n1] | Example 419: ¹H-NMR(400.0 MHz, d₆-DMSO): δ = 8.203(7.98); 7.907(4.15); 7.894(4.29); 7.744(0.42); 7.728(0.93); 7.723 (0.89); 7.706(1.74); 7.69(0.9); 7.685(1.06); 7.669(0.48); 7.362(0.52); 7.355 (3.12); 7.335(4.58); 7.315(2.64); 7.308(0.54); 7.118(4.26); 7.105(4.14); 5.757(7.5); 3.571(16); 2.955(1.37); 2.936(4.55); 2.918(4.68); 2.899(1.5); 2.52(0.43); 2.511(10.54); 2.507(22.54); 2.502(30.39); 2.498(22.08); 2.494 (10.85); 1.157(5.51); 1.138(11.85); 1.119(5.5); 1.102(0.38); 0(0.57) |
| 420 | 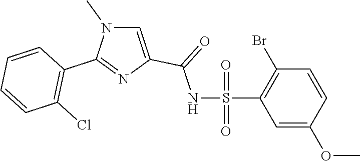 [SMILES: COc1ccc(Br)c(S(=O)(=O)NC(=O)c2cn(C)c(-c3ccccc3Cl)n2)c1] | Example 420: ¹H-NMR(400.0 MHz, d₆-DMSO): δ = 8.173(3.91); 7.714(2.71); 7.692(3.03); 7.683(1.19); 7.663(2.5); 7.648(2.97); 7.64(3.08); 7.634(1.12); 7.63(1.13); 7.616(1.25); 7.611(1.91); 7.609(1.95); 7.604(1.2); 7.589(2.39); 7.539(1.35); 7.536(1.26); 7.52(1.6); 7.502(0.59); 7.182(1.49); 7.174(1.42); 7.16(1.37); 7.152(1.33); 3.843(16); 3.53(14.93); 3.451(166.19); 2.714(0.39); 2.544(94.3); 2.527(1.15); 2.509(38.7); 2.504 (50); 2.5(36.08); 2.37(0.37); 0(5.12) |
| 421 | 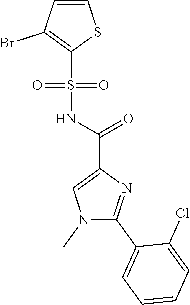 [SMILES: Cn1cc(C(=O)NS(=O)(=O)c2sccc2Br)nc1-c1ccccc1Cl] | Example 421: ¹H-NMR(400.0 MHz, d₆-DMSO): δ = 8.184(3.27); 7.947(2.24); 7.934(2.32); 7.702(0.99); 7.69(0.42); 7.683(1.73); 7.654(1.1); 7.65(1.2); 7.645(1.12); 7.637(1.67); 7.631(1.82); 7.627(1.41); 7.62(0.87); 7.556(1); 7.537(1.37); 7.519(0.52); 7.211(2.66); 7.198(2.55); 3.726(0.44); 3.551(16); 3.485(31.61); 2.544(61.39); 2.504(29.12); 0(2.56) |
| 422 | 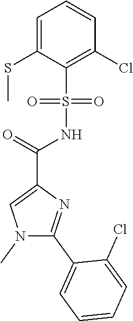 [SMILES: CSc1cccc(Cl)c1S(=O)(=O)NC(=O)c1cn(C)c(-c2ccccc2Cl)n1] | Example 422: ¹H-NMR(400.0 MHz, d₆-DMSO): δ = 8.143(2.61); 7.675(1.06); 7.655(2.08); 7.621(0.77); 7.604(1.29); 7.591 (1.15); 7.588(1.15); 7.573(1.85); 7.532(1.68); 7.514(3.01); 7.494(1.65); 7.401(1.63); 7.38(1.21); 7.36(1.82); 7.34(1.42); 3.52(16); 3.47(63.4); 2.542 (61.55); 2.502(36.39); 2.451(8.61); 2.367(0.32); 1.31(0.36); 0(0.66) |
| 423 | 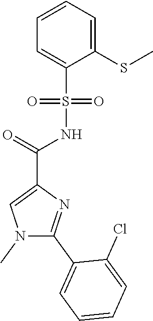 | Example 423: ¹H-NMR(400.0 MHz, d₆-DMSO): δ = 8.158(2.04); 8.049(1.04); 8.029(1.09); 7.668(0.81); 7.648(1.71); 7.633 (1.05); 7.614(1.25); 7.597(0.92); 7.576(0.54); 7.572(0.57); 7.566(0.7); 7.551 (1.11); 7.547(1.27); 7.525(0.98); 7.507(0.96); 7.484(1.27); 7.463(0.91); 7.389(0.65); 7.37(1.07); 7.351(0.54); 3.779(0.36); 3.508(16); 2.542(26.69); 2.54(27.94); 2.531(2.95); 2.501(15.38); 2.492(11.17); 0(0.33) |
| 424 | 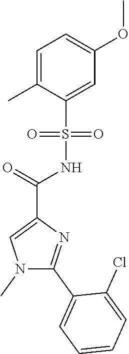 [SMILES: COc1ccc(C)c(S(=O)(=O)NC(=O)c2cn(C)c(-c3ccccc3Cl)n2)c1] | Example 424: ¹H-NMR(400.0 MHz, d₆-DMSO): δ = 8.108(4.42); 7.669(1.11); 7.649(2.27); 7.617(0.8); 7.612(0.89); 7.599(1.17); 7.595(1.43); 7.577(1.48); 7.563(2.15); 7.532(2.64); 7.526(3.91); 7.507(1.5); 7.489(0.56); 7.312(1.59); 7.291(2.07); 7.165(1.33); 7.158(1.3); 7.144(1.04); 7.137(1.01); 3.881(0.42); 3.803(16); 3.776(0.81); 3.68(1.18); 3.668(1.17); 3.656(1.19); 3.643(1.19); 3.62(1.23); 3.506(13.41); 3.419(0.42); 3.405 (0.39); 2.712(0.33); 2.542(68.62); 2.518(12.7); 2.507(22.46); 2.503(27.86); 2.498(19.91); 2.368(0.33); 0(1.11) |
| 425 | 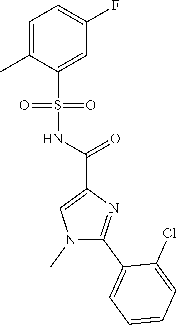 [SMILES: Cc1ccc(F)cc1S(=O)(=O)NC(=O)c1cn(C)c(-c2ccccc2Cl)n1] | Example 425: ¹H-NMR(400.0 MHz, d₆-DMSO): δ = 8.119(5.13); 7.758(1.37); 7.737(1.6); 7.677(1.29); 7.658(2.77); 7.629(0.93); 7.625(1.08); 7.607(1.78); 7.6(1.43); 7.596(1.32); 7.587(1.31); 7.581(2.63); 7.534(1.49); 7.516(1.8); 7.497(0.65); 7.443(3.25); 7.429(3.18); 7.426(3.14); 3.69(2.41); 3.581(0.9); 3.515(16); 2.712(0.38); 2.57(12.14); 2.543(79.95); 2.507(24.79); 2.503(31.95); 2.499(23.55); 2.368(0.42); 0(0.96) |
| 426 | 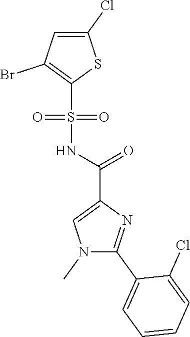 [SMILES: Cn1cc(C(=O)NS(=O)(=O)c2sc(Cl)cc2Br)nc1-c1ccccc1Cl] | Example 426: ¹H-NMR(400.0 MHz, d₆-DMSO): δ = 8.199(5.25); 7.74(2.11); 7.722(4.25); 7.71(1.49); 7.692(1.79); 7.672(0.71); 7.668(0.63); 7.596(1.44); 7.577(2.02); 7.558(0.9); 7.299(6.8); 4.148(0.6); 3.935(0.83); 3.595(16); 2.542(61.1); 2.503(20.19); 0(1.17) |
| 427 | 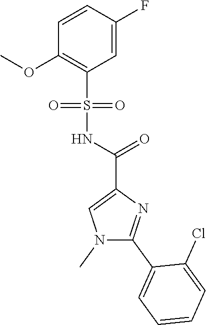 [SMILES: COc1ccc(F)cc1S(=O)(=O)NC(=O)c1cn(C)c(-c2ccccc2Cl)n1] | Example 427: ¹H-NMR(400.0 MHz, d₆-DMSO): δ = 8.162(4.53); 7.673(1.37); 7.666(1.4); 7.654(2.93); 7.645(1.52); 7.637(1.45); 7.62(0.81); 7.616(0.91); 7.598(1.52); 7.587(0.84); 7.578(2.2); 7.559(3.21); 7.544(0.89); 7.53(1.67); 7.511(1.6); 7.493(0.58); 7.291(1.25); 7.281(1.31); 7.268(1.1); 7.258(1.08); 3.841(15.2); 3.513(16); 3.336(0.55); 2.712(0.39); 2.542(69.17); 2.503(32.64); 2.368(0.33); 0(0.54) |
| 428 | 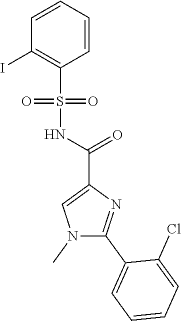 [SMILES: Cn1cc(C(=O)NS(=O)(=O)c2ccccc2I)nc1-c1ccccc1Cl] | Example 428: ¹H-NMR(400.0 MHz, d₆-DMSO): δ = 8.184(4.95); 8.178(2.22); 8.161(1.96); 8.158(1.91); 8.125(1.97); 8.123 (1.95); 8.105(2.14); 8.103(2); 7.678(1.26); 7.676(1.2); 7.658(3.69); 7.64 (1.93); 7.627(1.13); 7.622(2.05); 7.609(1.4); 7.604(2.24); 7.602(2.27); 7.598 (1.36); 7.583(2.71); 7.534(1.53); 7.531(1.47); 7.515(1.78); 7.497(0.7); 7.494 (0.63); 7.365(0.97); 7.361(0.99); 7.346(1.66); 7.342(1.64); 7.327(0.86); 7.323(0.82); 3.841(0.93); 3.654(2.34); 3.528(16); 2.542(76.21); 2.526(0.68); 2.507(20.93); 2.503(27.56); 2.498(19.97); 2.368(0.34); 0(1.16) |
| 429 | 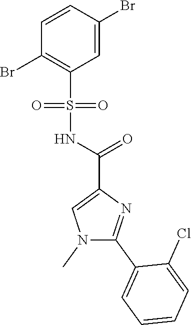 [SMILES: Cn1cc(C(=O)NS(=O)(=O)c2cc(Br)ccc2Br)nc1-c1ccccc1Cl] | Example 429: ¹H-NMR(400.0 MHz, d₆-DMSO): δ = 8.198(3.42); 8.196(3.25); 8.179(5.39); 7.71(9.32); 7.692(2.94); 7.671(3.33); 7.652(4.38); 7.632(0.94); 7.628(0.6); 7.566(1.34); 7.563(1.31); 7.544(1.84); 7.529(0.75); 7.526(0.7); 4.387(0.41); 4.262(0.57); 4.241(0.58); 4.191(0.57); 4.168(0.56); 3.561(16); 2.543(61.05); 2.508(12.62); 2.504(16.74); 2.499 (12.22); 0(0.69) |
| 430 | 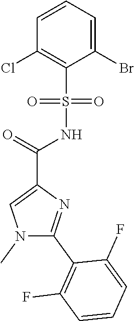 [SMILES: Cn1cc(C(=O)NS(=O)(=O)c2c(Cl)cccc2Br)nc1-c1c(F)cccc1F] | Example 430: ¹H-NMR(400.0 MHz, d₆-DMSO): δ = 8.24(4.54); 7.867(1.73); 7.847(1.9); 7.741(0.65); 7.737(0.64); 7.72(1.2); 7.703(0.7); 7.699(0.76); 7.686(1.77); 7.667(2.01); 7.489(1.58); 7.469(2.71); 7.449(1.21); 7.366(2.04); 7.346(3.38); 7.326(1.75); 3.83(0.67); 3.591(16); 2.543(43.17); 2.507(26.48); 2.503(34.46); 2.499(25.57); 0(0.59) |
| 431 | 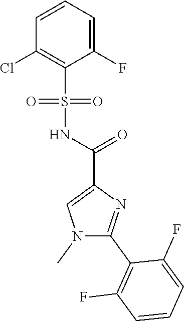 [SMILES: Cn1cc(C(=O)NS(=O)(=O)c2c(F)cccc2Cl)nc1-c1c(F)cccc1F] | Example 431: ¹H-NMR(400.0 MHz, d₆-DMSO): δ = 8.238(3.64); 7.741(0.66); 7.737(0.62); 7.72(1.21); 7.704(1.03); 7.698(0.83); 7.691(0.63); 7.684(1.27); 7.67(1.1); 7.663(0.74); 7.65(0.65); 7.509(1.85); 7.489(1.5); 7.457(0.88); 7.43(1.03); 7.408(0.75); 7.367(2.1); 7.346(3.34); 7.326(1.78); 3.765(0.52); 3.59(16); 3.525(6.28); 2.712(0.52); 2.542(120.77); 2.525(1.01); 2.507(35.23); 2.503(45.54); 2.499(32.55); 2.369(0.53) |
| 432 | 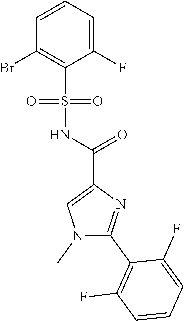 [SMILES: Cn1cc(C(=O)NS(=O)(=O)c2c(F)cccc2Br)nc1-c1c(F)cccc1F] | Example 432: ¹H-NMR(400.0 MHz, d₆-DMSO): δ = 8.256(6.43); 7.759(0.38); 7.741(0.93); 7.721(1.6); 7.705(2.91); 7.685(3.22); 7.612(0.83); 7.598(0.92); 7.591(1.53); 7.578(1.55); 7.571(0.85); 7.557 (0.77); 7.488(1.42); 7.46(1.55); 7.439(1.03); 7.368(2.56); 7.348(4.39); 7.327 (2.2); 3.598(16); 2.545(54.93); 2.506(11.94) |
| 433 | 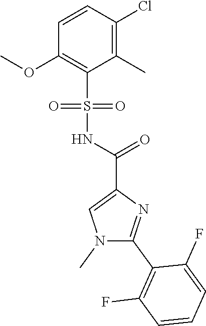 [SMILES: COc1ccc(Cl)c(C)c1S(=O)(=O)NC(=O)c1cn(C)c(-c2c(F)cccc2F)n1] | Example 433: ¹H-NMR(400.0 MHz, d₆-DMSO): δ = 8.217(4.45); 7.725(3); 7.703(3.09); 7.691(0.78); 7.687(0.83); 7.67(0.36); 7.36(2.21); 7.34(3.58); 7.32(1.91); 7.151(2.27); 7.129(2.13); 3.811(16); 3.626(0.37); 3.574(12.66); 3.543(0.76); 3.35(34.44); 2.727(14.46); 2.672 (0.59); 2.542(18.39); 2.507(71.87); 2.503(95.37); 2.498(70.65); 2.33(0.56); 0(4.04) |
| 434 | 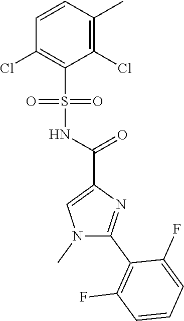 [SMILES: Cc1ccc(Cl)c(S(=O)(=O)NC(=O)c2cn(C)c(-c3c(F)cccc3F)n2)c1Cl] | Example 434: ¹H-NMR(400.0 MHz, d₆-DMSO): δ = 8.234(3.57); 7.735(0.63); 7.731(0.6); 7.714(1.16); 7.697(0.64); 7.693(0.7); 7.628(1.33); 7.608(2.51); 7.566(3.1); 7.545(1.62); 7.362(2); 7.342(3.23); 7.321(1.7); 3.757(0.64); 3.584(16); 3.417(0.58); 3.405(0.54); 2.712(0.42); 2.542(93.39); 2.526(0.89); 2.508(29.17); 2.503(38.34); 2.499(27.94); 2.377 (12.41) |
| 435 | 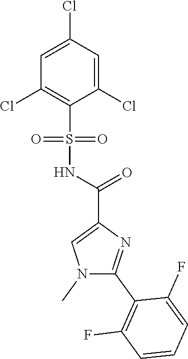 [SMILES: Cn1cc(C(=O)NS(=O)(=O)c2c(Cl)cc(Cl)cc2Cl)nc1-c1c(F)cccc1F] | Example 435: ¹H-NMR(400.0 MHz, d₆-DMSO): δ = 8.232(3.81); 7.851(9.65); 7.758(0.58); 7.754(0.6); 7.737(1.07); 7.716(0.68); 7.379(1.83); 7.359(3.12); 7.338(1.59); 3.607(16); 3.522(9.38); 2.543(16.12); 2.507(31.16); 2.504(40.67) |
| 436 | 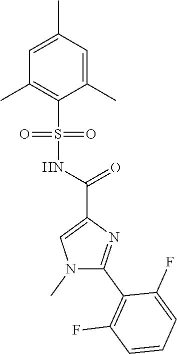 [SMILES: Cc1cc(C)c(S(=O)(=O)NC(=O)c2cn(C)c(-c3c(F)cccc3F)n2)c(C)c1] | Example 436: ¹H-NMR(400.0 MHz, d₆-DMSO): δ = 8.166(3.17); 7.722(0.45); 7.717(0.44); 7.7(0.81); 7.68(0.49); 7.35(1.39); 7.33(2.2); 7.309(1.17); 7.039(4.42); 3.558(8.19); 3.362(4.62); 2.629(16); 2.542(60.37); 2.526(0.63); 2.507(23.02); 2.503(29.1); 2.498(20.96); 2.264 (7.67) |
| 437 | 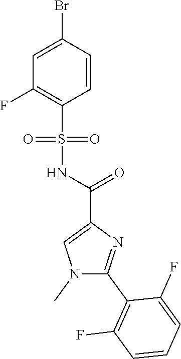 [SMILES: Cn1cc(C(=O)NS(=O)(=O)c2ccc(Br)cc2F)nc1-c1c(F)cccc1F] | Example 437: ¹H-NMR(400.0 MHz, d₆-DMSO): δ = 8.21(3.57); 7.91(1.37); 7.889(2.7); 7.869(1.72); 7.842(1.52); 7.82(1.46); 7.817(1.5); 7.756(0.36); 7.738(0.83); 7.734(0.81); 7.717(1.55); 7.7(0.93); 7.696(1.07); 7.689(1.93); 7.685(1.9); 7.668(1.54); 7.664(1.53); 7.364(2.72); 7.344(4.33); 7.323(2.31); 3.756(0.33); 3.58(16); 3.437(36.94); 2.712(0.96); 2.672(0.71); 2.668(0.56); 2.542(214.04); 2.525(2.26); 2.507(85.45); 2.503 (112.58); 2.499(81.61); 2.368(0.91); 2.334(0.5); 2.33(0.65); 1.235(0.35); 0(0.99) |
| 438 | 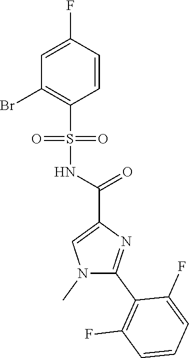 [SMILES: Cn1cc(C(=O)NS(=O)(=O)c2ccc(F)cc2Br)nc1-c1c(F)cccc1F] | Example 438: ¹H-NMR(400.0 MHz, d₆-DMSO): δ = 8.252(4.18); 8.242(1.51); 8.234(1.39); 8.219(1.3); 7.864(1.18); 7.857(1.23); 7.843(1.26); 7.836(1.22); 7.733(0.65); 7.729(0.63); 7.712(1.2); 7.695(0.68); 7.691(0.74); 7.557(0.68); 7.551(0.65); 7.535(1.16); 7.53(1.04); 7.515(0.69); 7.508(0.61); 7.36(2.09); 7.339(3.38); 7.319(1.81); 3.756(0.44); 3.582(16); 3.499(9.39); 2.713(0.51); 2.543(103.25); 2.508(34.83); 2.504(44.93); 2.5 (32.79); 2.369(0.47); 0(0.33) |
| 439 | 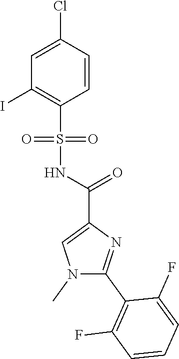 [SMILES: Cn1cc(C(=O)NS(=O)(=O)c2ccc(Cl)cc2I)nc1-c1c(F)cccc1F] | Example 439: ¹H-NMR(400.0 MHz, d₆-DMSO): δ = 8.264(6.25); 8.21(3.79); 8.205(3.9); 8.148(3.82); 8.127(4.32); 7.752(2.43); 7.747(2.15); 7.731(2.6); 7.725(2.12); 7.715(1.62); 7.694(0.97); 7.678(0.43); 7.362(2.64); 7.342(4.43); 7.322(2.3); 4.012(0.51); 3.762(1.66); 3.704(1.8); 3.7(1.8); 3.588(16); 2.543(9.16); 2.507(25.83); 2.504(32.93); 2.5(24.32); 0 (1.26) |
| 440 | 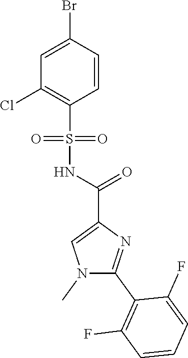 [SMILES: Cn1cc(C(=O)NS(=O)(=O)c2ccc(Br)cc2Cl)nc1-c1c(F)cccc1F] | Example 440: ¹H-NMR(400.0 MHz, d₆-DMSO): δ = 8.234(3.14); 8.057(2.37); 8.036(2.94); 7.979(2.25); 7.975(2.74); 7.848 (1.46); 7.843(1.48); 7.826(1.22); 7.822(1.25); 7.736(0.61); 7.732(0.64); 7.715(1.14); 7.694(0.72); 7.362(1.94); 7.342(3.23); 7.321(1.7); 3.755(0.4); 3.583(16); 3.507(9.75); 2.712(0.51); 2.542(107.82); 2.526(1.31); 2.508 (36.64); 2.503(48.33); 2.499(36.22); 2.369(0.48); 0(0.53) |
| 441 | 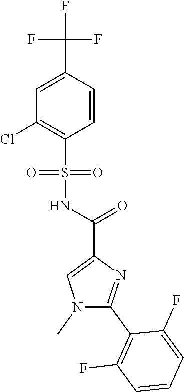 [SMILES: Cn1cc(C(=O)NS(=O)(=O)c2ccc(C(F)(F)F)cc2Cl)nc1-c1c(F)cccc1F] | Example 441: ¹H-NMR(400.0 MHz, d₆-DMSO): δ = 8.347(2.19); 8.326(2.5); 8.236(4.56); 8.087(3.21); 7.995(1.77); 7.974(1.56); 7.77(0.37); 7.753(0.83); 7.749(0.82); 7.732(1.54); 7.715(0.85); 7.711(0.92); 7.694(0.38); 7.375(2.63); 7.355(4.39); 7.334(2.27); 3.601(16); 3.475(24.56); 3.34(0.4); 2.713(0.77); 2.672(0.45); 2.543(177.33); 2.526(1.76); 2.508 (61.16); 2.503(80.63); 2.499(58.86); 2.369(0.8); 2.33(0.5); 0(0.38) |
| 442 | 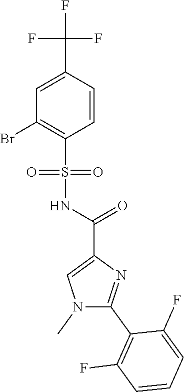 [SMILES: Cn1cc(C(=O)NS(=O)(=O)c2ccc(C(F)(F)F)cc2Br)nc1-c1c(F)cccc1F] | Example 442: ¹H-NMR(400.0 MHz, d₆-DMSO): δ = 8.365(1.77); 8.344(2.06); 8.254(4.1); 8.217(2.68); 8.04(1.46); 8.02(1.29); 7.755(0.7); 7.751(0.67); 7.734(1.24); 7.717(0.72); 7.713(0.76); 7.376(2.15); 7.356(3.58); 7.336(1.84); 3.606(16); 3.564(6.93); 2.714(0.33); 2.544(69.56); 2.528(0.98); 2.509(21.31); 2.505(27.25); 2.501(20.2); 2.371(0.34); 0(0.62) |
| 443 | 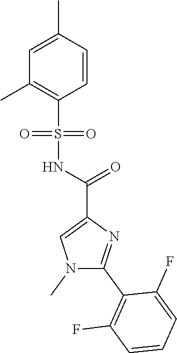 [SMILES: Cc1ccc(S(=O)(=O)NC(=O)c2cn(C)c(-c3c(F)cccc3F)n2)c(C)c1] | Example 443: ¹H-NMR(400.0 MHz, d₆-DMSO): δ = 8.172(5.28); 7.915(2.92); 7.894(3.12); 7.746(0.35); 7.728(0.84); 7.724 (0.79); 7.707(1.52); 7.691(0.85); 7.686(0.91); 7.67(0.38); 7.357(2.56); 7.337 (4.18); 7.316(2.22); 7.249(1.72); 7.228(1.65); 7.206(3.21); 3.74(0.4); 3.708 (0.4); 3.703(0.41); 3.565(16); 3.376(27.11); 3(0.33); 2.718(0.79); 2.678 (0.57); 2.564(15.74); 2.548(159.13); 2.531(1.82); 2.513(70.94); 2.509 (91.93); 2.505(67.16); 2.374(0.82); 2.345(14.61) |
| 444 | 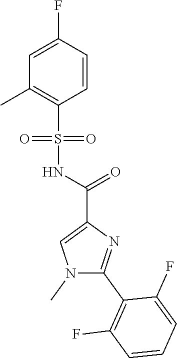 [SMILES: Cc1cc(F)ccc1S(=O)(=O)NC(=O)c1cn(C)c(-c2c(F)cccc2F)n1] | Example 444: ¹H-NMR(400.0 MHz, d₆-DMSO): δ = 8.183(4.5); 8.107(1.02); 8.092(1.36); 8.083(1.39); 8.069(1.13); 7.749(0.35); 7.732(0.83); 7.728(0.81); 7.711(1.46); 7.69(0.89); 7.673(0.38); 7.359(2.5); 7.339(4.09); 7.318(3.06); 7.314(3.29); 7.291(2.68); 7.275(1.06); 3.86(0.33); 3.758(0.53); 3.745(0.63); 3.569(16); 3.418(14.45); 3.098(0.57); 3.015(0.37); 3.001(0.38); 2.718(0.79); 2.678(0.59); 2.615(14.18); 2.548(153.82); 2.513 (70.26); 2.509(87.45); 2.375(0.75); 2.335(0.55) |
| 445 | 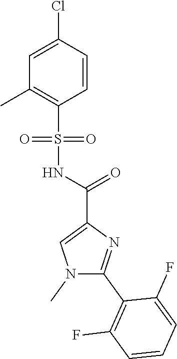 [SMILES: Cc1cc(Cl)ccc1S(=O)(=O)NC(=O)c1cn(C)c(-c2c(F)cccc2F)n1] | Example 445: ¹H-NMR(400.0 MHz, d₆-DMSO): δ = 8.179(4.44); 8.024(2.7); 8.013(0.8); 8.001(3.02); 7.745(0.35); 7.728(0.81); 7.724(0.77); 7.707(1.47); 7.69(0.83); 7.686(0.91); 7.669(0.39); 7.534(2.48); 7.529(3.02); 7.525(2.91); 7.518(2.23); 7.355(2.54); 7.335(3.98); 7.314 (2.19); 3.739(0.57); 3.565(16); 3.446(5.13); 2.712(0.69); 2.672(0.45); 2.668 (0.34); 2.596(14.13); 2.542(163.17); 2.525(1.77); 2.507(58.73); 2.502 (77.53); 2.498(57.03); 2.368(0.78); 2.334(0.43); 2.329(0.53); 0(2.14) |
| 446 | 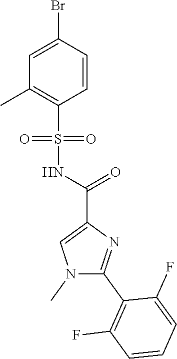 [SMILES: Cc1cc(Br)ccc1S(=O)(=O)NC(=O)c1cn(C)c(-c2c(F)cccc2F)n1] | Example 446: ¹H-NMR(400.0 MHz, d₆-DMSO): δ = 8.176(3.19); 7.939(2.43); 7.93(0.53); 7.925(0.51); 7.916(2.9); 7.745(0.38); 7.727(0.83); 7.707(1.48); 7.686(1.06); 7.671(4.14); 7.656(1.77); 7.355 (2.42); 7.335(4.02); 7.314(2.07); 3.788(0.38); 3.74(0.54); 3.565(16); 3.408 (11.06); 3.045(0.4); 2.995(0.36); 2.712(0.93); 2.672(0.78); 2.588(14.65); 2.542(190.02); 2.525(2.5); 2.506(100.46); 2.502(126.28); 2.498(91.61); 2.368(0.94); 2.329(0.79); 1.236(0.38); 0(3.51) |
| 447 | 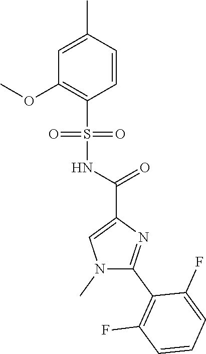 [SMILES: COc1cc(C)ccc1S(=O)(=O)NC(=O)c1cn(C)c(-c2c(F)cccc2F)n1] | Example 447: ¹H-NMR(400.0 MHz, d₆-DMSO): δ = 8.206(5.05); 7.769(2.83); 7.749(3.06); 7.727(0.71); 7.722(0.66); 7.706 (1.29); 7.689(0.69); 7.685(0.78); 7.668(0.34); 7.358(2.28); 7.338(3.45); 7.318(1.9); 7.31(0.41); 7.052(2.94); 6.961(1.59); 6.941(1.52); 3.822(16); 3.568(11.82); 3.34(10.21); 2.712(0.41); 2.542(103.13); 2.525(0.91); 2.511 (15.09); 2.507(30.7); 2.502(40.46); 2.498(29.16); 2.375(11.83); 0(1.12) |
| 448 | 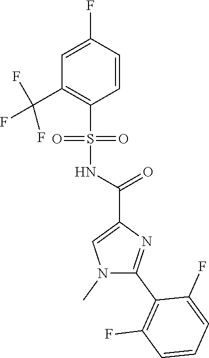 [SMILES: Cn1cc(C(=O)NS(=O)(=O)c2ccc(F)cc2C(F)(F)F)nc1-c1c(F)cccc1F] | Example 448: ¹H-NMR(400.0 MHz, d₆-DMSO): δ = 8.427(0.9); 8.414(0.98); 8.405(1.02); 8.392(0.98); 8.226(3.75); 7.926(0.93); 7.919(1.07); 7.902(0.97); 7.896(1.03); 7.838(0.58); 7.831(0.53); 7.816 (0.96); 7.796(0.58); 7.79(0.48); 7.743(0.61); 7.739(0.59); 7.722(1.12); 7.705 (0.65); 7.701(0.67); 7.367(1.95); 7.347(3.25); 7.327(1.7); 3.588(16); 3.42 (0.45); 3.409(0.44); 2.542(27.35); 2.507(38.74); 2.503(50.65); 2.499 (37.05); 0(1.15) |
| 449 | 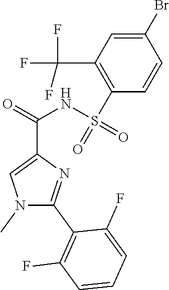 [SMILES: Cn1cc(C(=O)NS(=O)(=O)c2ccc(Br)cc2C(F)(F)F)nc1-c1c(F)cccc1F] | Example 449: ¹H-NMR(400.0 MHz, d₆-DMSO): δ = 8.244(1.23); 8.219(4.28); 8.183(1.92); 8.161(1.11); 8.142(2.49); 7.747 (0.67); 7.73(1.09); 7.709(0.77); 7.373(1.77); 7.352(3.05); 7.332(1.61); 3.596(16); 3.52(9.89); 2.673(0.38); 2.542(22.01); 2.503(61.09); 2.5(53.8); 2.331(0.38); 0(0.93) |
| 450 | 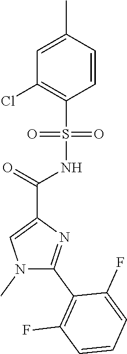 [SMILES: Cc1ccc(S(=O)(=O)NC(=O)c2cn(C)c(-c3c(F)cccc3F)n2)c(Cl)c1] | Example 450: ¹H-NMR(400.0 MHz, d₆-DMSO): δ = 8.183(5.36); 8.026(2.72); 8.014(0.82); 8.003(3.03); 7.745(0.33); 7.728(0.8); 7.724(0.8); 7.707(1.48); 7.69(0.83); 7.687(0.92); 7.67(0.39); 7.536(2.59); 7.531(3.24); 7.526(3.16); 7.52(2.43); 7.355(2.52); 7.335(4.1); 7.315(2.22); 3.908(0.33); 3.846(0.42); 3.742(0.78); 3.72(0.81); 3.566(16); 3.388(1.11); 3.263(0.53); 3.205(0.39); 2.712(0.56); 2.597(14.57); 2.542(114.65); 2.507 (36.01); 2.503(47.48); 2.499(35.93); 2.369(0.59); 2.33(0.32); 0(1.08) |
| 451 | 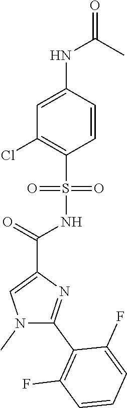 [SMILES: CC(=O)Nc1ccc(S(=O)(=O)NC(=O)c2cn(C)c(-c3c(F)cccc3F)n2)c(Cl)c1] | Example 451: ¹H-NMR(400.0 MHz, d₆-DMSO): δ = 10.52(3.05); 8.235(5.28); 8.087(3.01); 8.065(3.39); 7.974(3.33); 7.969 (3.42); 7.741(0.33); 7.724(0.78); 7.703(1.4); 7.682(0.86); 7.665(0.37); 7.636(1.79); 7.631(1.76); 7.613(1.64); 7.608(1.61); 7.353(2.39); 7.332 (3.96); 7.312(2.05); 3.851(0.38); 3.804(0.46); 3.746(0.62); 3.572(14.65); 3.528(0.92); 3.489(0.83); 3.393(0.64); 3.312(0.42); 3.289(0.4); 3.268(0.35); 2.543(58.65); 2.526(0.69); 2.503(38.56); 2.368(0.33); 2.099(16); 0(1.23) |
| 452 | 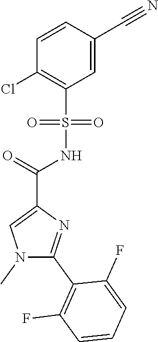 [SMILES: Cn1cc(C(=O)NS(=O)(=O)c2cc(C#N)ccc2Cl)nc1-c1c(F)cccc1F] | Example 452: ¹H-NMR(400.0 MHz, d₆-DMSO): δ = 8.471(2.48); 8.466(2.55); 8.234(3.59); 8.146(1.19); 8.141(1.18); 8.125 (1.42); 8.12(1.41); 7.878(2.48); 7.857(2.14); 7.766(0.54); 7.762(0.51); 7.745 (0.97); 7.728(0.55); 7.724(0.59); 7.384(1.68); 7.364(2.86); 7.343(1.46); 3.612(16); 2.712(0.45); 2.542(108.57); 2.525(0.83); 2.507(28.79); 2.503 (38.06); 2.499(27.66); 2.369(0.48); 0(0.62) |
| 453 | 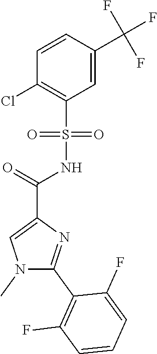 [SMILES: Cn1cc(C(=O)NS(=O)(=O)c2cc(C(F)(F)F)ccc2Cl)nc1-c1c(F)cccc1F] | Example 453: ¹H-NMR(400.0 MHz, d₆-DMSO): δ = 8.35(2.32); 8.345(2.37); 8.243(3.9); 8.074(1.03); 8.069(1); 8.053(1.31); 8.048(1.26); 7.91(2.04); 7.889(1.62); 7.766(0.69); 7.762(0.65); 7.745(1.22); 7.728(0.7); 7.724(0.73); 7.386(2.15); 7.365(3.56); 7.345(1.83); 3.613 (15.22); 3.563(16); 2.718(0.51); 2.548(121.27); 2.532(1.33); 2.513(40.15); 2.509(51.88); 2.505(37.84); 2.374(0.54) |
| 454 | 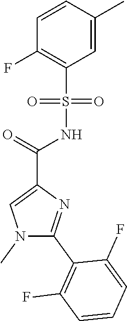 [SMILES: Cc1ccc(F)c(S(=O)(=O)NC(=O)c2cn(C)c(-c3c(F)cccc3F)n2)c1] | Example 454: ¹H-NMR(400.0 MHz, d₆-DMSO): δ = 8.216(2.85); 7.775(1.39); 7.762(1.32); 7.758(1.34); 7.733(0.81); 7.729 (0.79); 7.712(1.44); 7.691(0.9); 7.675(0.39); 7.551(0.95); 7.362(2.52); 7.342(5.13); 7.321(3.38); 7.295(0.94); 3.751(0.44); 3.576(16); 3.436 (29.02); 2.718(0.83); 2.678(0.6); 2.574(0.59); 2.548(185.49); 2.531(2.06); 2.513(75.5); 2.509(97.36); 2.505(70.61); 2.39(11.97); 2.375(1.12); 2.336 (0.62) |
| 455 | 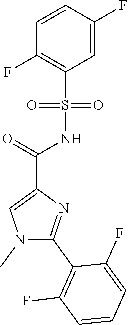 [SMILES: Cn1cc(C(=O)NS(=O)(=O)c2cc(F)ccc2F)nc1-c1c(F)cccc1F] | Example 455: ¹H-NMR(400.0 MHz, d₆-DMSO): δ = 8.229(4.74); 7.769(0.38); 7.758(0.75); 7.751(1.54); 7.737(1.72); 7.731(2.4); 7.716(1.2); 7.71(0.94); 7.693(0.39); 7.658(0.57); 7.644(0.68); 7.637(1.04); 7.627(0.73); 7.616(0.76); 7.608(0.37); 7.542(0.78); 7.531(0.81); 7.518(1.26); 7.508(1.25); 7.495(0.57); 7.485(0.52); 7.376(2.49); 7.355(4.1); 7.335(2.15); 3.596(16); 3.495(24.17); 2.718(0.67); 2.678(0.34); 2.548(156.97); 2.532 (1.7); 2.513(48.33); 2.509(62.78); 2.504(45.59); 2.374(0.7); 2.336(0.39) |
| 456 | 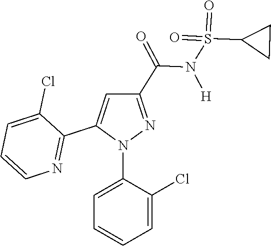 [SMILES: O=C(NS(=O)(=O)C1CC1)c1cc(-c2ncccc2Cl)n(-c2ccccc2Cl)n1] | Example 456: ¹H-NMR(400.0 MHz, d₆-DMSO): δ = 12.135(2.19); 8.308(4.19); 8.305(4.57); 8.296(4.48); 8.293(4.51); 8.097 (4.13); 8.094(4.21); 8.077(4.57); 8.073(4.34); 7.656(0.42); 7.647(2.41); 7.639(3.32); 7.635(2.12); 7.632(2.39); 7.624(3.24); 7.616(0.68); 7.566(16); 7.55(0.32); 7.535(0.82); 7.525(1.51); 7.519(2.42); 7.511(7.97); 7.504 (11.58); 7.495(6.36); 7.49(4.48); 7.48(3.81); 7.473(0.63); 7.422(4.29); 7.41 (4.08); 7.401(3.98); 7.39(3.98); 5.754(4.65); 3.335(20.9); 3.153(0.66); 3.141 (1.4); 3.134(1.54); 3.122(2.6); 3.11(1.58); 3.102(1.53); 3.09(0.72); 2.671 (0.41); 2.524(1.43); 2.511(25.05); 2.506(50.32); 2.502(68.52); 2.497(51.37); 2.493(25.25); 2.329(0.4); 1.211(0.88); 1.196(3.08); 1.189(3.94); 1.182 (5.05); 1.17(2.01); 1.154(2.83); 1.145(3.6); 1.139(3.63); 1.125(3.86); 1.12 (2.94); 1.105(0.8); 0.008(0.85); 0(19.69); −0.008(0.75) |
| 457 | 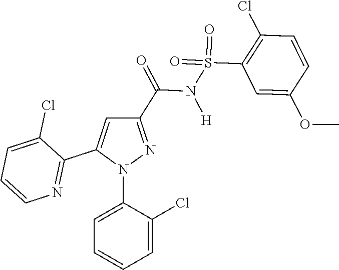 [SMILES: COc1ccc(Cl)c(S(=O)(=O)NC(=O)c2cc(-c3ncccc3Cl)n(-c3ccccc3Cl)n2)c1] | Example 457: ¹H-NMR(400.0 MHz, d₆-DMSO): δ = 8.298(1.75); 8.294(1.94); 8.286(1.88); 8.283(1.93); 8.084(1.72); 8.081(1.8); 8.064(1.9); 8.06(1.88); 7.656(3.03); 7.649(3.17); 7.624(1.17); 7.615(3.33); 7.601(1.74); 7.593(3.77); 7.587(5.64); 7.529(0.41); 7.518(0.89); 7.511 (1.23); 7.504(3.26); 7.498(1.59); 7.494(4.11); 7.487(2.1); 7.482(1.35); 7.477 (1.72); 7.47(1.77); 7.412(1.75); 7.4(1.66); 7.391(1.63); 7.38(1.64); 7.33(1.6); 7.322(1.52); 7.308(1.4); 7.3(1.35); 5.755(0.91); 3.871(16); 3.171(3.03); 2.511(9.9); 2.507(19.71); 2.503(26.71); 2.498(20.33); 0.008(0.43); 0(9.84); −0.008(0.42) |
| 458 | 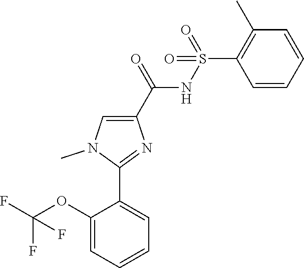 [SMILES: Cc1ccccc1S(=O)(=O)NC(=O)c1cn(C)c(-c2ccccc2OC(F)(F)F)n1] | Example 458: ¹H-NMR(400.0 MHz, d₆-DMSO): δ = 8.124(6.21); 8.031(2.05); 8.011(2.16); 7.734(0.63); 7.73(0.84); 7.71(1.77); 7.694(2.53); 7.691(2.27); 7.678(2.37); 7.586(4.36); 7.568(5.43); 7.549 (2.24); 7.455(1.26); 7.436(2.02); 7.417(0.92); 7.399(2.18); 7.38(1.8); 3.535 (16); 2.602(13.9); 2.506(21.74); 2.502(30.08); 2.498(24.38); 0(26.63) |
| 459 | 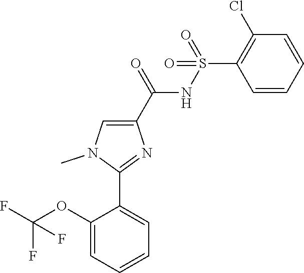 [SMILES: Cn1cc(C(=O)NS(=O)(=O)c2ccccc2Cl)nc1-c1ccccc1OC(F)(F)F] | Example 459: ¹H-NMR(400.0 MHz, d₆-DMSO): δ = 8.187(5.35); 8.161(1.76); 8.158(1.98); 8.141(1.92); 8.138(2.05); 7.744 (0.69); 7.739(0.89); 7.721(1.74); 7.719(1.79); 7.708(2.2); 7.7(2.03); 7.691 (3.32); 7.672(1.81); 7.668(1.91); 7.658(2.35); 7.655(3.16); 7.639(1.14); 7.635(0.85); 7.62(1.31); 7.616(1.19); 7.594(4.09); 7.583(1.25); 7.575(3.72); 7.557(0.9); 5.756(7.05); 3.554(16); 2.507(19.17); 2.502(26.97); 2.498 (21.26); 0.008(1.09); 0(30.44) |
| 460 | 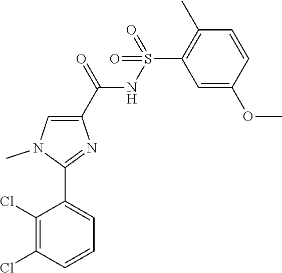 | Example 460: ¹H-NMR(400.0 MHz, d₆-DMSO): δ = 8.126(4.11); 7.871(1.38); 7.866(1.55); 7.852(1.56); 7.848(1.68); 7.575 (0.53); 7.57(0.89); 7.556(2.53); 7.551(2.38); 7.546(2.72); 7.528(4.49); 7.522 (3.07); 7.508(0.8); 7.314(1.72); 7.292(2.23); 7.168(1.4); 7.162(1.39); 7.148 (1.1); 7.141(1.07); 3.803(16); 3.509(13.6); 3.455(0.5); 3.374(0.63); 3.329 (0.65); 2.67(0.61); 2.505(75.85); 2.501(100.16); 2.497(77.24); 2.328(0.61); 2.074(1.04); 0.007(2.44); 0(58.02) |
| 461 | 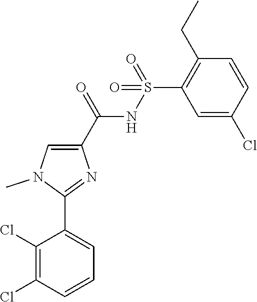 [SMILES: CCc1ccc(Cl)cc1S(=O)(=O)NC(=O)c1cn(C)c(-c2cccc(Cl)c2Cl)n1] | Example 461: ¹H-NMR(400.0 MHz, d₆-DMSO): δ = 8.136(6.52); 8.126(0.39); 7.973(3.48); 7.967(3.66); 7.884(1.86); 7.879 (2.12); 7.864(2.12); 7.86(2.26); 7.696(1.61); 7.69(1.57); 7.676(1.96); 7.67 (1.94); 7.591(0.89); 7.586(1.28); 7.572(3.1); 7.567(2.77); 7.557(3); 7.537 (3.28); 7.518(1.13); 7.488(3.1); 7.467(2.55); 3.521(15.74); 3.057(1); 3.039 (3.29); 3.02(3.36); 3.002(1.08); 2.525(0.58); 2.507(28.81); 2.502(38.5); 2.498(28.99); 2.075(16); 1.164(4.01); 1.145(8.63); 1.126(3.92); 0.008 (1.09); 0(30.53); −0.008(1.53) |
| 462 | 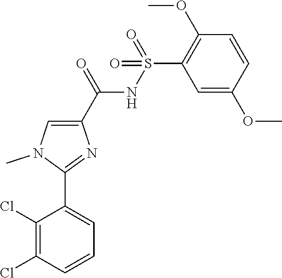 | Example 462: ¹H-NMR(400.0 MHz, d₆-DMSO): δ = 8.168(1.65); 7.874(0.74); 7.868(0.79); 7.856(0.82); 7.85(0.87); 7.567(0.4); 7.554(1.56); 7.549(2.22); 7.532(1.21); 7.513(0.37); 7.38(1.41); 7.372(1.62); 7.269(0.47); 7.262(0.4); 7.246(0.81); 7.239(0.77); 7.192(1.45); 7.17(0.8); 3.787(16); 3.515(6.96); 3.335(0.48); 3.323(0.45); 2.506(31.37); 2.502 (41.44); 2.497(31.36); 0.008(1.12); 0(26.16); −0.008(1.37) |
| 463 | 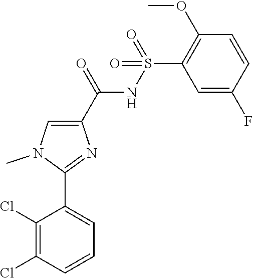 [SMILES: COc1ccc(F)cc1S(=O)(=O)NC(=O)c1cn(C)c(-c2cccc(Cl)c2Cl)n1] | Example 463: ¹H-NMR(400.0 MHz, d₆-DMSO): δ = 8.181(4.98); 7.874(1.53); 7.868(1.64); 7.856(1.68); 7.85(1.8); 7.664(1.31); 7.656(1.58); 7.644(1.42); 7.636(1.57); 7.591(0.71); 7.582(0.59); 7.568 (1.88); 7.56(1.24); 7.554(3.44); 7.549(5.35); 7.54(0.93); 7.531(2.52); 7.512 (0.79); 7.294(1.34); 7.284(1.4); 7.27(1.18); 7.26(1.13); 3.842(16); 3.517 (14.51); 3.487(0.35); 2.671(0.36); 2.506(45.57); 2.502(59.84); 2.497(44.89); 2.328(0.35); 1.507(0.37); 0.008(1.6); 0(40.12); −0.008(1.92) |
| 464 | 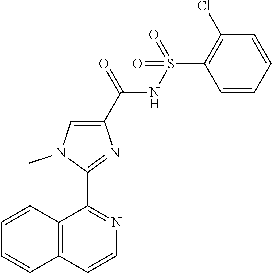 [SMILES: Cn1cc(C(=O)NS(=O)(=O)c2ccccc2Cl)nc1-c1nccc2ccccc12] | Example 464: ¹H-NMR(400.0 MHz, d₆-DMSO): δ = 8.795(1.63); 8.774(1.73); 8.648(3.23); 8.634(3.44); 8.287(2.71); 8.192 (1.53); 8.188(1.7); 8.172(1.65); 8.168(1.76); 8.084(1.63); 8.064(1.98); 7.984 (2.28); 7.97(2.19); 7.866(0.98); 7.849(1.82); 7.831(1.05); 7.828(1.06); 7.76 (1.08); 7.758(1.15); 7.74(1.89); 7.736(1.59); 7.719(1.87); 7.695(2.23); 7.691 (2.56); 7.675(0.85); 7.649(1.02); 7.645(0.9); 7.629(1.33); 7.612(0.64); 7.608 (0.59); 3.888(16); 3.349(2.03); 2.675(0.82); 2.67(1.17); 2.666(0.89); 2.524 (3.75); 2.51(74.34); 2.506(152.3); 2.501(205.61); 2.497(155.18); 2.493 (79.22); 2.333(0.9); 2.328(1.25); 2.324(0.96); 0.146(1); 0.008(8.68); 0 (225.36); −0.008(10.48); −0.15(1.03) |
| 465 | 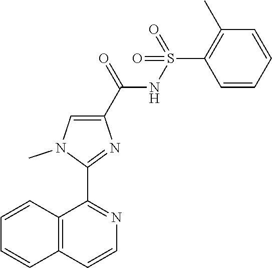 [SMILES: Cc1ccccc1S(=O)(=O)NC(=O)c1cn(C)c(-c2nccc3ccccc23)n1] | Example 465: ¹H-NMR(400.0 MHz, d₆-DMSO): δ = 8.795(1.7); 8.774(1.79); 8.644(3.25); 8.63(3.48); 8.228(4.12); 8.083(1.66); 8.06(2.77); 8.037(1.81); 7.98(2.34); 7.966(2.28); 7.87(0.87); 7.868(0.98); 7.85(1.83); 7.833(1.09); 7.83(1.12); 7.769(1.07); 7.766(1.18); 7.748(1.61); 7.745(1.26); 7.73(0.82); 7.728(0.83); 7.601(0.68); 7.585(1.51); 7.582(1.6); 7.566(1.03); 7.564(1.06); 7.473(1); 7.454(1.63); 7.436(0.76); 7.421(1.79); 7.402(1.45); 3.873(16); 3.336(3.2); 2.676(0.38); 2.671(0.51); 2.667(0.43); 2.644(12.69); 2.524(1.39); 2.511(28.56); 2.507(58.68); 2.502(79.33); 2.498 (60.12); 2.493(31.18); 2.333(0.35); 2.329(0.49); 2.324(0.37); 2.074(0.79); 0.146(0.42); 0.008(3.75); 0(95.66); −0.008(4.96); −0.15(0.44) |
| 466 | 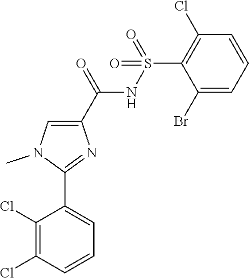 [SMILES: Cn1cc(C(=O)NS(=O)(=O)c2c(Cl)cccc2Br)nc1-c1cccc(Cl)c1Cl] | Example 466: ¹H-NMR(400.0 MHz, d₆-DMSO): δ = 8.185(5.86); 8.156(0.37); 7.898(1.86); 7.893(2.23); 7.878(2.17); 7.874 (2.47); 7.844(1.92); 7.842(2.33); 7.824(2.18); 7.822(2.55); 7.663(1.88); 7.661(2.26); 7.643(2.33); 7.641(2.63); 7.622(1.3); 7.618(1.64); 7.603(2.69); 7.599(2.71); 7.568(2.59); 7.548(3.27); 7.529(1.31); 7.459(1.91); 7.438 (3.29); 7.418(1.48); 5.755(1.7); 4.054(1.19); 3.571(0.41); 3.544(16); 2.671 (0.37); 2.506(41.55); 2.501(58.72); 2.497(47.37); 2.328(0.34); 0.008(2.3); 0 (56.73) |
| 467 | 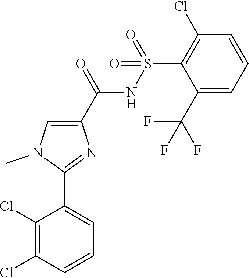 [SMILES: Cn1cc(C(=O)NS(=O)(=O)c2c(Cl)cccc2C(F)(F)F)nc1-c1cccc(Cl)c1Cl] | Example 467: ¹H-NMR(400.0 MHz, d₆-DMSO): δ = 8 166(2.97); 7.99(1.31); 7.97(1.61); 7.924(1.3); 7.906(3.32); 7.89(1.74); 7.886(1.82); 7.779(0.99); 7.76(1.57); 7.74(0.69); 7.644(1.04); 7.64(1.18); 7.624(1.94); 7.621(1.83); 7.577(1.95); 7.558(2.58); 7.538(1.09); 4.051 (2.24); 3.866(0.34); 3.552(16); 3.53(3.82); 2.674(0.92); 2.67(1.26); 2.666 (0.94); 2.505(164.29); 2.501(217.32); 2.496(162.83); 2.332(0.97); 2.328 (1.33); 2.323(1); 2.073(2.19); 1.506(0.33); 0.146(1.05); 0.008(10.53); 0 (244.26); −0.009(11.53); −0.15(1.09) |
| 468 | 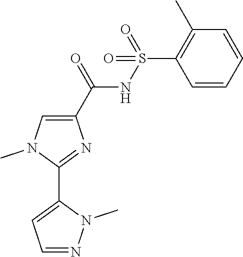 [SMILES: Cc1ccccc1S(=O)(=O)NC(=O)c1cn(C)c(-c2ccnn2C)n1] | Example 468: ¹H-NMR(400.0 MHz, d₆-DMSO): δ = 8.088(4.01); 8.026(1.45); 8.007(1.52); 7.587(0.64); 7.574(3.86); 7.569 (4.73); 7.552(0.93); 7.549(0.92); 7.457(0.9); 7.438(1.41); 7.419(0.64); 7.398(1.55); 7.38(1.27); 6.744(3.23); 6.739(3.26); 4.04(16); 3.97(0.58); 3.726(13); 3.321(0.52); 2.67(0.34); 2.612(10.61); 2.51(18.91); 2.506(38.65); 2.501(53.33); 2.497(40.54); 1.512(1.37); 0.008(1.86); 0(48.34); −0.008 (1.97) |
| 469 | 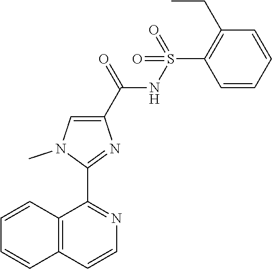 [SMILES: CCc1ccccc1S(=O)(=O)NC(=O)c1cn(C)c(-c2nccc3ccccc23)n1] | Example 469: ¹H-NMR(400.0 MHz, d₆-DMSO): δ = 8.789(1.73); 8.768(1.82); 8.643(3.39); 8.629(3.61); 8.222(4.39); 8.084 (1.74); 8.063(2.24); 8.058(2.07); 8.055(1.99); 8.038(1.79); 8.035(1.9); 7.98 (2.42); 7.967(2.34); 7.871(0.94); 7.869(1.01); 7.854(1.45); 7.851(1.91); 7.834(1.15); 7.831(1.12); 7.765(1.13); 7.763(1.21); 7.744(1.67); 7.741 (1.28); 7.727(0.86); 7.724(0.84); 7.652(0.71); 7.649(0.73); 7.63(1.64); 7.614 (1.05); 7.611(1.06); 7.474(2.17); 7.466(1.43); 7.455(1.76); 7.447(1.87); 7.428(0.86); 5.756(0.64); 3.874(16); 3.326(0.98); 3.113(1.04); 3.095(3.17); 3.076(3.24); 3.058(1.07); 2.675(0.38); 2.671(0.53); 2.666(0.39); 2.524 (1.55); 2.51(30.02); 2.506(62.83); 2.502(87.74); 2.497(66.8); 2.493(33.22); 2.333(0.4); 2.328(0.52); 2.324(0.4); 1.218(3.91); 1.199(8.58); 1.18(3.83); 0.146(0.36); 0.008(3.03); 0(82.96); −0.008(3.39); −0.15(0.36) |
| 470 | 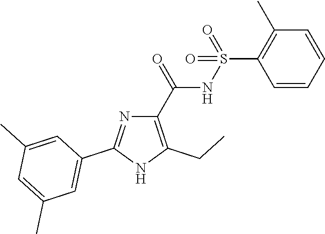 [SMILES: CCc1[nH]c(-c2cc(C)cc(C)c2)nc1C(=O)NS(=O)(=O)c1ccccc1C] | Example 470: ¹H-NMR(400.0 MHz, d₆-DMSO): δ = 8.033(0.97); 8.015(1.02); 8.013(1.01); 7.664(2.67); 7.564(0.35); 7.546 (0.82); 7.528(0.55); 7.443(0.55); 7.425(0.87); 7.405(0.4); 7.388(0.96); 7.37 (0.78); 7.069(1.49); 3.772(0.44); 3.748(0.47); 3.725(0.47); 3.634(0.4); 3.613 (0.37); 3.598(0.34); 2.86(0.37); 2.842(1.04); 2.823(1.08); 2.805(0.41); 2.631 (7.81); 2.524(0.51); 2.51(15.73); 2.506(33.08); 2.502(44.35); 2.497(32.21); 2.492(15.62); 2.343(14.54); 2.074(16); 1.149(1.88); 1.13(4.15); 1.111(1.88); 0(0.58) |
| 471 | 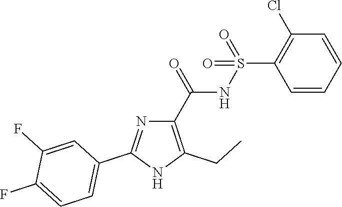 [SMILES: CCc1[nH]c(-c2ccc(F)c(F)c2)nc1C(=O)NS(=O)(=O)c1ccccc1Cl] | Example 471: ¹H-NMR(400.0 MHz, d₆-DMSO): δ = 13.142(0.72); 8.172(0.89); 8.162(3.42); 8.158(3.89); 8.142(3.99); 8.139 (3.85); 8.126(0.87); 7.889(1.26); 7.868(1.52); 7.702(0.56); 7.684(1.73); 7.66(3.72); 7.656(4.07); 7.64(2.26); 7.618(3.29); 7.597(2.95); 7.584(1.34); 7.58(1.12); 7.57(0.93); 3.992(0.34); 3.584(2.97); 3.542(3.13); 3.442(2.04); 3.318(0.89); 3.186(0.71); 2.866(1.44); 2.847(4.34); 2.828(4.48); 2.809 (1.56); 2.675(1.08); 2.67(1.48); 2.666(1.12); 2.524(2.72); 2.519(4.56); 2.51 (90.44); 2.506(195.4); 2.501(266.22); 2.497(193.16); 2.492(93.19); 2.338 (0.65); 2.333(1.24); 2.328(1.66); 2.324(1.28); 2.074(11.59); 1.154(7.04); 1.135(16); 1.116(6.97); 0(4.04) |
| 472 | 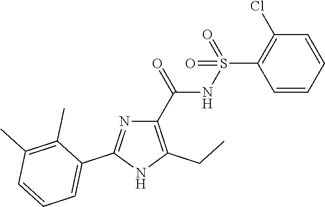 [SMILES: CCc1[nH]c(-c2cccc(C)c2C)nc1C(=O)NS(=O)(=O)c1ccccc1Cl] | Example 472: ¹H-NMR(400.0 MHz, d₆-DMSO): δ = 13.513(0.34); 8.102(1.85); 8.082(2.27); 7.535(4.14); 7.511(1.13); 7.504 (0.93); 7.492(1.18); 7.482(0.82); 7.472(0.47); 7.355(2.54); 7.336(3.34); 7.254(1.63); 7.236(2.09); 7.217(0.87); 3.824(0.34); 3.739(0.51); 3.483 (19.27); 3.284(0.6); 3.225(0.42); 3.182(0.6); 2.991(0.99); 2.973(2.5); 2.954 (2.55); 2.935(1.01); 2.76(0.34); 2.752(0.35); 2.674(1.81); 2.67(2.46); 2.666 (1.86); 2.58(0.46); 2.568(0.56); 2.556(0.64); 2.523(6.61); 2.51(144.41); 2.505(301.24); 2.501(403.44); 2.496(290.08); 2.492(138.13); 2.332(1.95); 2.328(2.74); 2.322(2.85); 2.314(16); 2.244(10.52); 2.073(3.31); 1.198(3.93); 1.18(8.21); 1.161(3.8); 0(5.21) |
| 473 | 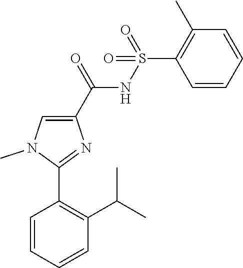 [SMILES: Cc1ccccc1S(=O)(=O)NC(=O)c1cn(C)c(-c2ccccc2C(C)C)n1] | Example 473: ¹H-NMR(400.0 MHz, d₆-DMSO): δ = 8.133(2.27); 8.059(5.06); 8.018(2.12); 8.001(2.25); 7.561(0.83); 7.542 (2.31); 7.522(2.53); 7.512(2.99); 7.502(5.15); 7.487(0.66); 7.435(1.3); 7.416(2.08); 7.397(0.99); 7.383(2.32); 7.364(1.88); 7.32(4.77); 7.313(4.47); 7.311(3.83); 4.039(0.4); 3.928(0.45); 3.894(0.46); 3.89(0.46); 3.872(0.47); 3.834(0.47); 3.622(0.39); 3.447(18.06); 2.762(0.48); 2.745(1.07); 2.728 (1.46); 2.711(1.09); 2.694(0.48); 2.675(0.37); 2.671(0.4); 2.601(15.16); 2.506(42.85); 2.502(56.37); 2.497(42.39); 2.328(0.34); 2.073(2.1); 1.116 (16); 1.098(15.81); 0(61.59) |
| 474 | 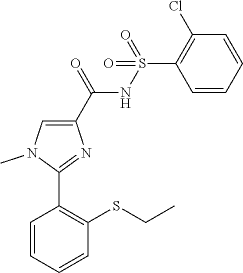 [SMILES: CCSc1ccccc1-c1nc(C(=O)NS(=O)(=O)c2ccccc2Cl)cn1C] | Example 474: ¹H-NMR(400.0 MHz, d₆-DMSO): δ = 8.136(1.8); 8.132(2.09); 8.117(4.61); 7.657(0.35); 7.641(1.24); 7.619(2.99); 7.614(3.06); 7.598(0.97); 7.581(1.29); 7.576(1.34); 7.558(2.72); 7.539 (5.49); 7.524(0.72); 7.432(1.54); 7.414(2.51); 7.355(1.19); 7.35(1.15); 7.334(1.47); 7.321(0.74); 7.315(0.7); 5.753(2.08); 3.889(1.35); 3.66(0.39); 3.485(16); 2.971(1.43); 2.953(4.53); 2.935(4.62); 2.916(1.51); 2.671(0.44); 2.506(55.09); 2.501(74.67); 2.497(58.35); 2.332(0.35); 2.328(0.45); 1.194 (4.89); 1.175(10.12); 1.157(4.71); 0.146(0.35); 0.008(3.54); 0(76.43); −0.008(3.71); −0.15(0.36) |
| 475 | 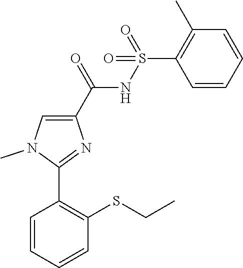 [SMILES: CCSc1ccccc1-c1nc(C(=O)NS(=O)(=O)c2ccccc2C)cn1C] | Example 475: ¹H-NMR(400.0 MHz, d₆-DMSO): δ = 8.063(4.94); 8.023(2.18); 8.003(2.33); 7.57(0.89); 7.552(2.49); 7.532(3.06); 7.516(5.13); 7.499(0.78); 7.441(1.3); 7.422(2.09); 7.403(1.11); 7.39(3.38); 7.372(3.98); 7.332(1.33); 7.328(1.26); 7.313(1.7); 7.298(0.72); 7.292(0.68); 4.044(0.39); 3.767(0.33); 3.636(0.36); 3.461(16); 2.963(1.41); 2.945(4.25); 2.926(4.32); 2.908(1.46); 2.67(0.39); 2.606(14.34); 2.502(57.26); 2.328 (0.35); 2.073(2.75); 1.194(4.55); 1.176(9.14); 1.158(4.35); 0(47.84) |
| 476 | 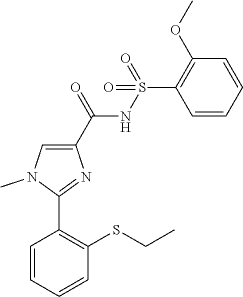 [SMILES: CCSc1ccccc1-c1nc(C(=O)NS(=O)(=O)c2ccccc2OC)cn1C] | Example 476: ¹H-NMR(400.0 MHz, d₆-DMSO): δ = 8.132(0.9); 8.095(4.95); 7.902(1.53); 7.898(1.69); 7.882(1.72); 7.878(1.83); 7.683(0.7); 7.679(0.73); 7.661(1.29); 7.644(0.89); 7.639(0.85); 7.533(1.26); 7.529(1.47); 7.518(3.02); 7.514(4.01); 7.5(0.59); 7.493(0.44); 7.387(0.95); 7.384(1.07); 7.366(2.27); 7.335(1.18); 7.329(1.15); 7.319(1.07); 7.314 (1.38); 7.31(0.9); 7.301(0.62); 7.295(0.64); 7.239(1.97); 7.218(1.77); 7.161 (1.08); 7.143(1.89); 7.123(0.95); 3.85(16); 3.464(14.55); 3.452(1.06); 3.436 (1.95); 3.34(1.29); 3.222(0.47); 3.14(0.79); 2.963(1.31); 2.945(4.13); 2.926 (4.21); 2.908(1.37); 2.674(0.45); 2.67(0.62); 2.665(0.46); 2.523(1.37); 2.51 (33.11); 2.505(71.02); 2.501(100.31); 2.496(74.77); 2.492(35.66); 2.332 (0.43); 2.328(0.59); 2.323(0.43); 1.507(3.73); 1.205(0.48); 1.197(4.39); 1.187(1.07); 1.179(9.24); 1.169(0.63); 1.161(4.23); 0.146(0.33); 0.008 (2.41); 0(75.64); −0.008(2.57) |
| 477 | 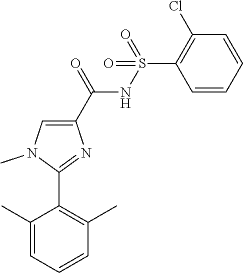 [SMILES: Cc1cccc(C)c1-c1nc(C(=O)NS(=O)(=O)c2ccccc2Cl)cn1C] | Example 477: ¹H-NMR(400.0 MHz, d₆-DMSO): δ = 8.144(3.82); 8.131(1.81); 8.124(1.1); 8.108(1.06); 8.105(1.08); 7.908(0.96); 7.616(0.72); 7.612(0.72); 7.596(2.4); 7.592(2.01); 7.577(0.55); 7.562(0.79); 7.557(0.6); 7.543(0.85); 7.537(0.64); 7.526(0.45); 7.521(0.41); 7.393(0.68); 7.374(1.32); 7.355(1.07); 7.314(0.51); 7.295(0.44); 7.222(2.67); 7.203 (2.12); 7.182(1.05); 7.163(0.77); 3.691(1.51); 3.635(1.23); 3.514(0.37); 3.495(0.32); 3.42(9.62); 3.35(1.39); 3.336(3.75); 2.675(0.32); 2.67(0.47) 2.666(0.35); 2.524(0.91); 2.51(26.24); 2.506(57.3); 2.501(81.82); 2.496 (61.37); 2.492(29.54); 2.332(0.35); 2.328(0.49); 2.323(0.38); 2.072(1.15); 1.993(16); 1.965(2.36); 1.955(5.97); 1.512(10.51); 0.146(0.41); 0.008(2.82); 0(93.65); −0.009(3.28); −0.15(0.42) |
| 478 | 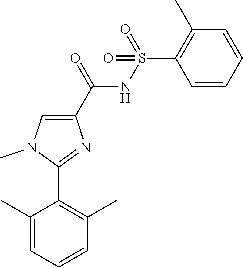 [SMILES: Cc1ccccc1S(=O)(=O)NC(=O)c1cn(C)c(-c2c(C)cccc2C)n1] | Example 478: ¹H-NMR(400.0 MHz, d₆-DMSO): δ = 8.076(1.23); 8.017(0.98); 7.997(1.01); 7.9(0.8); 7.555(0.34); 7.536(0.82); 7.517(0.54); 7.431(0.55); 7.411(0.9); 7.392(0.43); 7.375(1); 7.356(1.4); 7.337(1.26); 7.318(1.09); 7.197(2.68); 7.178(2.48); 7.162(0.48); 3.372 (8.99); 3.345(0.74); 3.333(2.28); 2.675(0.36); 2.67(0.47); 2.666(0.36); 2.601(7.84); 2.524(1.37); 2.51(25.98); 2.506(53.75); 2.501(74.27); 2.497 (55.3); 2.492(26.72); 2.332(0.33); 2.328(0.45); 2.324(0.32); 1.977(16); 1.963(1.16); 1.953(3.4); 1.511(5.87); 0.008(2.06); 0(54.07); −0.008(2) |
| 479 | 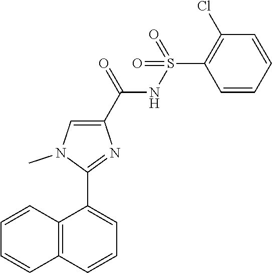 [SMILES: Cn1cc(C(=O)NS(=O)(=O)c2ccccc2Cl)nc1-c1cccc2ccccc12] | Example 479: ¹H-NMR(400.0 MHz, d₆-DMSO): δ = 8.239(5.62); 8.161(4.91); 8.14(5.68); 8.137(5.46); 8.119(0.4); 8.111(0.37); 8.096(0.4); 8.075(2.36); 8.055(2.86); 8.037(0.55); 8.004(0.91); 7.736(2.01); 7.719(3.5); 7.682(2.67); 7.663(5); 7.643(8.21); 7.618(3.37); 7.597(5.66); 7.577(4.32); 7.56(1.73); 4.65(0.33); 4.604(0.34); 4.561(0.35); 4.55(0.36); 4.524(0.36); 4.483(0.35); 4.478(0.35); 4.47(0.34); 4.452(0.34); 3.522(16); 3.503(1); 3.472(2.59); 2.672(0.35); 2.504(59.86); 2.501(60.35); 2.328(0.36); 2.075(8.91); 2.074(8.89); 1.533(7.14); 0.002(46.67); 0(47.15) |
| 480 | 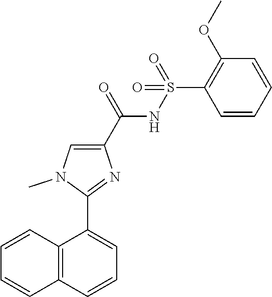 [SMILES: COc1ccccc1S(=O)(=O)NC(=O)c1cn(C)c(-c2cccc3ccccc23)n1] | Example 480: ¹H-NMR(400.0 MHz, d₆-DMSO): δ = 8.222(5.44); 8.134(4.97); 8.116(1.68); 8.064(1.53); 8.045(1.56); 8(1.01); 7.925(1.57); 7.921(1.75); 7.906(1.74); 7.902(1.82); 7.722(1.2); 7.699(2.9); 7.684(2.86); 7.68(3.51); 7.671(2.67); 7.66(1.08); 7.651(2.43); 7.638(0.75); 7.633(1.29); 7.63(1.37); 7.624(0.8); 7.61(1.52); 7.607(1.35); 7.598(0.45); 7.588(2.16); 7.584(1.82); 7.568(1.33); 7.564(1.24); 7.551(0.71); 7.548(0.5); 7.26(2.08); 7.24(1.89); 7.175(1.12); 7.157(2.03); 7.139(1.01); 7.137(1); 3.875(16); 3.512(14.27); 3.471(2.81); 2.524(0.51); 2.511(14.2); 2.506 (30.32); 2.502(42.75); 2.497(32.8); 2.493(16.47); 2.073(15.8); 1.532(7.37); 0.008(1.97); 0(61.72); −0.008(2.41) |
| 481 | 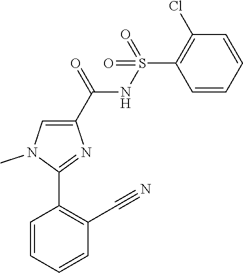 [SMILES: Cn1cc(C(=O)NS(=O)(=O)c2ccccc2Cl)nc1-c1ccccc1C#N] | Example 481: ¹H-NMR(400.0 MHz, d₆-DMSO): δ = 8.039(0.67); 8.02(0.68); 7.99(2.03); 7.856(0.67); 7.84(0.52); 7.836(0.49); 7.758(0.93); 7.751(0.63); 7.738(0.64); 7.732(0.79); 7.713(0.32); 3.669 (0.66); 3.654(0.54); 3.632(5.77); 3.323(4.79); 2.675(0.44); 2.67(0.58); 2.666(0.44); 2.524(1.69); 2.51(34.17); 2.506(70.96); 2.501(98.64); 2.497 (75.18); 2.492(37.55); 2.332(0.42); 2.328(0.57); 2.324(0.44); 1.518(16); 0.146(0.54); 0.008(4.22); 0(122.23); −0.008(5.21); −0.15(0.54) |
| 482 | 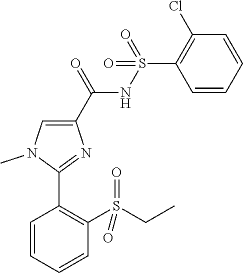 [SMILES: CCS(=O)(=O)c1ccccc1-c1nc(C(=O)NS(=O)(=O)c2ccccc2Cl)cn1C] | Example 482: ¹H-NMR(400.0 MHz, d₆-DMSO): δ = 8.161(3.19); 8.158(3.48); 8.138(11.41); 8.078(2.93); 8.075(2.86); 8.059 (3.87); 8.056(3.71); 7.924(0.99); 7.92(1.2); 7.905(3.06); 7.901(3.29); 7.887 (3.28); 7.882(3.04); 7.878(2.92); 7.874(3.24); 7.859(3.2); 7.855(3.31); 7.84 (1.23); 7.836(1.05); 7.744(3.59); 7.74(3.81); 7.725(2.89); 7.722(3.15); 7.716(1.03); 7.7(2.4); 7.696(2.29); 7.682(2.99); 7.678(3.06); 7.668(4.07); 7.665(5.27); 7.649(2.01); 7.645(1.4); 7.626(2.21); 7.622(1.94); 7.606(2.84); 7.589(1.45); 7.585(1.32); 3.72(0.32); 3.686(0.35); 3.656(0.39); 3.632(0.4); 3.592(0.55); 3.578(0.46); 3.547(0.52); 3.528(0.54); 3.502(2.17); 3.484 (6.32); 3.466(6.43); 3.447(2.33); 3.417(30.52); 3.327(0.42); 3.272(0.37); 3.237(0.46); 2.675(0.6); 2.67(0.82); 2.666(0.61); 2.524(1.91); 2.519(2.84); 2.51(43.16); 2.506(91.76); 2.501(128.98); 2.497(97.15); 2.492(47.25); 2.333 (0.59); 2.328(0.79); 2.324(0.6); 2.073(1.24); 1.505(0.57); 1.101(7.02); 1.083 (16); 1.064(6.89); 0.146(0.76); 0.008(5.78); 0(180.8); −0.008(6.73); −0.15 (0.77) |
| 483 | 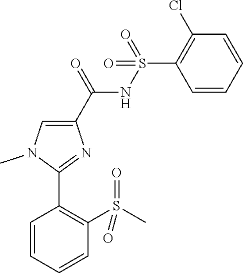 [SMILES: Cn1cc(C(=O)NS(=O)(=O)c2ccccc2Cl)nc1-c1ccccc1S(C)(=O)=O] | Example 483: ¹H-NMR(400.0 MHz, d₆-DMSO): δ = 8.314(0.36); 8.155(2.27); 8.137(3.64); 8.127(2.9); 8.109(2.63); 8.104(2.54); 8.099(1); 8.095(0.86); 7.921(2.31); 7.911(0.62); 7.908(0.77); 7.892(1.98); 7.889(2.13); 7.875(4.13); 7.87(3.81); 7.856(2.23); 7.852(2.28); 7.837(0.72); 7.832(1.01); 7.828(0.71); 7.729(2.18); 7.725(2.26); 7.708(2.1); 7.693(1.23); 7.674(2.07); 7.657(2.54); 7.64(1); 7.62(1.08); 7.601(1.52); 7.582(0.71); 5.753(2.03); 3.609(0.46); 3.595(0.58); 3.519(0.88); 3.421(24.59); 3.369 (7.25); 3.347(20.16); 3.259(1.11); 3.243(0.99); 3.226(1.01); 3.172(0.62); 2.674(1.01); 2.67(1.31); 2.505(149.41); 2.501(204.79); 2.497(159.19); 2.332 (0.9); 2.328(1.2); 2.323(0.9); 1.988(0.98); 1.506(16); 1.236(1.55); 1.175 (0.51); 0.146(0.84); 0.008(7.47); 0(175.15); −0.008(9.24); −0.15(0.86) |
| 484 | 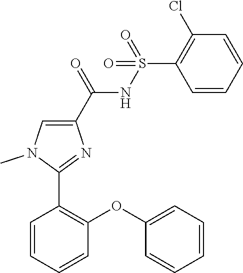 [SMILES: Cn1cc(C(=O)NS(=O)(=O)c2ccccc2Cl)nc1-c1ccccc1Oc1ccccc1] | Example 484: ¹H-NMR(400.0 MHz, d₆-DMSO): δ = 8.133(1.1); 8.121(1.77); 8.118(1.9); 8.102(1.94); 8.098(1.98); 8.066(4.68); 7.846(0.39); 7.652(0.41); 7.649(0.43); 7.632(1.38); 7.615(1.92); 7.611 (2.11); 7.605(3.97); 7.601(4.91); 7.586(2.83); 7.583(2.9); 7.576(1.46); 7.572(2.1); 7.552(3.02); 7.536(1.62); 7.534(1.63); 7.396(2.22); 7.377(4.07); 7.357(2.95); 7.311(1.34); 7.292(2.3); 7.273(1.05); 7.177(1.38); 7.159(2.33); 7.14(1); 7.03(4.16); 7.01(3.95); 6.982(2.54); 6.961(2.38); 3.629(16); 3.595 (1.1); 2.671(0.34); 2.506(42.62); 2.502(58.28); 2.497(45.03); 2.328(0.35); 2.073(1.02); 1.49(2.83); 0.008(1.12); 0(29.43) |
| 485 | 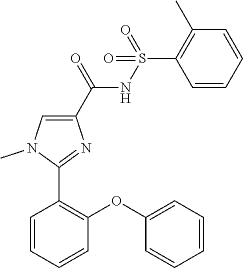 [SMILES: Cc1ccccc1S(=O)(=O)NC(=O)c1cn(C)c(-c2ccccc2Oc2ccccc2)n1] | Example 485: ¹H-NMR(400.0 MHz, d₆-DMSO): δ = 8.01(4.83); 7.983(1.93); 7.581(1.49); 7.577(1.89); 7.562(2.38); 7.558(2.88); 7.55(1.07); 7.54(1.89); 7.535(1.91); 7.524(1.3); 7.515(1.29); 7.51(1.14); 7.429(1.09); 7.41(1.75); 7.391(1.17); 7.385(2.31); 7.374(2.58); 7.366(3.85); 7.364(3.84); 7.355(2.01); 7.346(2.94); 7.299(1.32); 7.28(2.25); 7.261(1.02); 7.164(1.41); 7.146(2.38); 7.127(1.02); 7.009(4.03); 6.99(3.58); 6.976(2.45); 6.956(2.23); 3.604(16); 2.67(0.52); 2.666(0.4); 2.585(13.27); 2.51(28.21); 2.506(57.42); 2.501(79.25); 2.497(59.89); 2.333(0.34); 2.328(0.45); 2.324 (0.34); 2.073(2.15); 1.49(1.6); 0.008(1.5); 0(38.26); −0.008(1.38) |
| 486 | 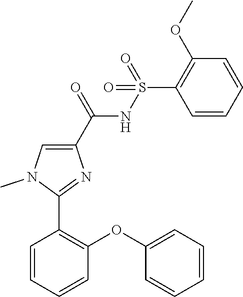 [SMILES: COc1ccccc1S(=O)(=O)NC(=O)c1cn(C)c(-c2ccccc2Oc2ccccc2)n1] | Example 486: ¹H-NMR(400.0 MHz, d₆-DMSO): δ = 8.051(4.14); 7.888(1.58); 7.884(1.68); 7.868(1.71); 7.864(1.74); 7.844(0.4); 7.673(0.71); 7.669(0.72); 7.651(1.37); 7.633(0.87); 7.629(0.82); 7.577(1.31); 7.573(1.72); 7.558(2.33); 7.554(2.53); 7.537(1.53); 7.518(1.09); 7.514(0.92); 7.386(1.87); 7.366(3.26); 7.346(2.52); 7.309(1.19); 7.29(2); 7.271(0.91); 7.218(2.09); 7.197(1.91); 7.16(1.27); 7.152(1.31); 7.142(2.17); 7.134(2.18); 7.123(0.96); 7.115(1.03); 7.007(3.55); 6.986(5.02); 6.965(2.02); 5.753(4.14); 3.802(16); 3.744(0.33); 3.61(14.86); 3.594(1.97); 3.568(1.13); 3.411(4.73); 2.676(0.39); 2.671(0.5); 2.667(0.38); 2.524(1.32); 2.506(53.35); 2.502 (73.22); 2.498(55.15); 2.329(0.42); 1.49(2.83); 0.008(0.62); 0(16.77); −0.008(0.58) |
| 487 | 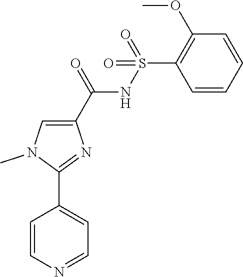 [SMILES: COc1ccccc1S(=O)(=O)NC(=O)c1cn(C)c(-c2ccncc2)n1] | Example 487: ¹H-NMR(601.6 MHz, d₆-DMSO): δ = 8.726(3.68); 8.716(3.43); 8.147(2.81); 7.904(1.48); 7.902(1.53); 7.891(1.6); 7.888(1.49); 7.816(4.14); 7.814(2.81); 7.808(2.84); 7.806(3.91); 7.675 (0.77); 7.663(1.38); 7.651(0.75); 7.231(1.99); 7.217(1.89); 7.159(1.09); 7.146(1.94); 7.134(0.95); 3.883(15.39); 3.863(0.71); 3.854(16); 3.402 (0.33); 3.308(6.68); 2.612(0.4); 2.521(0.8); 2.518(1.07); 2.503(42.46); 2.5 (55.52); 2.498(41.7); 2.387(0.38); 2.384(0.46); 2.071(3.29); 1.516(0.64); 0(0.89) |
| 488 | 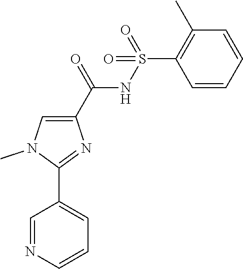 [SMILES: Cc1ccccc1S(=O)(=O)NC(=O)c1cn(C)c(-c2cccnc2)n1] | Example 488: ¹H-NMR(400.0 MHz, d₆-DMSO): δ = 8.982(1.79); 8.693(1.24); 8.683(1.23); 8.209(0.94); 8.204(1.46); 8.199 (0.98); 8.189(1.05); 8.184(1.56); 8.18(1.02); 8.132(0.76); 8.102(6.38); 8.03 (1.79); 8.012(1.82); 8.01(1.86); 7.584(0.9); 7.579(1.36); 7.566(2.97); 7.559 (1.36); 7.547(2.25); 7.455(1.08); 7.436(1.72); 7.417(0.77); 7.398(1.9); 7.379(1.59); 3.813(16); 2.67(0.39); 2.616(12.8); 2.524(1.01); 2.51(22.86); 2.506(46.62); 2.501(63.27); 2.497(48.31); 2.493(24.42); 2.328(0.39); 2.073 (2); 0.008(1.32); 0(34.73); −0.008(1.44) |
| 489 | 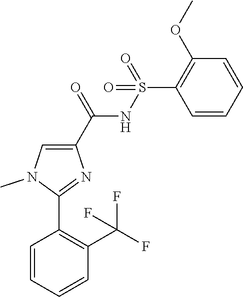 [SMILES: COc1ccccc1S(=O)(=O)NC(=O)c1cn(C)c(-c2ccccc2C(F)(F)F)n1] | Example 489: ¹H-NMR(400.0 MHz, d₆-DMSO): δ = 8.314(0.56); 8.148(3.05); 7.958(1.28); 7.94(1.65); 7.936(1.64); 7.904(1.59); 7.9(1.78); 7.884(1.7); 7.88(1.82); 7.86(0.46); 7.841(1.41); 7.825(2.25); 7.821(2.41); 7.803(1.29); 7.786(0.4); 7.688(0.76); 7.676(2); 7.661(1.52); 7.248(1.97); 7.227(1.81); 7.169(1.08); 7.149(1.98); 7.13(0.99); 3.832(16); 3.444(14.69); 3.32(5.15); 2.675(1.14); 2.67(1.61); 2.666(1.24); 2.54(2.82); 2.523(3.87); 2.506(196.71); 2.501(276.94); 2.497(212.64); 2.328(1.57); 2.324(1.26); 2.073(0.77); 1.235(1.24); 0.008(0.68); 0(19.99) |
| 490 | 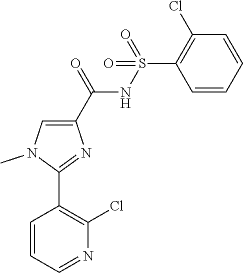 [SMILES: Cn1cc(C(=O)NS(=O)(=O)c2ccccc2Cl)nc1-c1cccnc1Cl] | Example 490: ¹H-NMR(400.0 MHz, d₆-DMSO): δ = 8.621(1.75); 8.616(1.92); 8.609(1.91); 8.604(1.9); 8.2(3.54); 8.157(1.62); 8.154(1.77); 8.138(1.79); 8.134(1.9); 8.1(1.64); 8.095(1.74); 8.081(1.88); 8.076(1.83); 7.713(0.36); 7.71(0.4); 7.693(1.23); 7.69(1.19); 7.676(1.48); 7.672(1.55); 7.659(2.69); 7.643(1.03); 7.635(2.02); 7.622(2.53); 7.616 (2.62); 7.604(2.76); 7.584(0.71); 7.58(0.66); 3.56(16); 2.675(0.33); 2.67 (0.44); 2.666(0.35); 2.541(28.09); 2.524(1.08); 2.51(27.41); 2.506(57.46); 2.502(80.39); 2.497(60.9); 2.333(0.37); 2.328(0.5); 2.324(0.37); 2.074 (5.44); 0.008(1.25); 0(35.66); −0.008(1.39) |
| 491 | 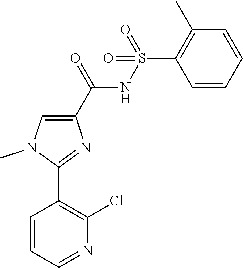 [SMILES: Cc1ccccc1S(=O)(=O)NC(=O)c1cn(C)c(-c2cccnc2Cl)n1] | Example 491: ¹H-NMR(601.6 MHz, d₆-DMSO): δ = 18.058(0.38); 14.922(0.36); 13.643(0.35); 11.16(0.41); 8.627(0.36); 8.618 (0.38); 8.612(2.13); 8.608(2.1); 8.604(2.18); 8.6(2.21); 8.592(0.39); 8.535 (0.42); 8.532(0.53); 8.524(0.45); 8.309(0.92); 8.127(1.16); 8.082(1.54); 8.079(1.47); 8.07(1.91); 8.066(1.52); 8.043(0.5); 8.022(1.22); 8.008(1.24); 7.976(0.36); 7.959(0.79); 7.948(0.43); 7.945(0.4); 7.624(2.45); 7.616(2.14); 7.611(2.1); 7.603(2.1); 7.598(0.47); 7.59(0.41); 7.586(0.4); 7.577(0.58); 7.561(1.04); 7.547(0.61); 7.442(0.79); 7.43(1.16); 7.416(0.62); 7.392(1.21); 7.379(1.04); 5.749(0.44); 3.922(0.35); 3.624(3.02); 3.545(16); 3.528(2.42); 3.442(0.39); 3.436(0.36); 3.43(0.37); 3.418(0.61); 3.408(0.55); 3.391(0.61); 3.385(0.81); 3.332(2.4); 3.304(3.82); 3.219(0.62); 3.196(0.39); 3.191(0.37); 2.615(1.53); 2.612(2.28); 2.607(11.92); 2.582(2.39); 2.521(3.37); 2.518 (4.11); 2.515(4.23); 2.506(101.75); 2.503(230.83); 2.5(323.6); 2.497 (233.94); 2.494(105.53); 2.388(1.3); 2.384(1.78); 2.381(1.28); 1.575(0.57); 1.51(6.45); 1.481(0.44); 0.005(0.5); 0(30.09); −0.006(1.35); −3.282(0.37) |
| 492 | 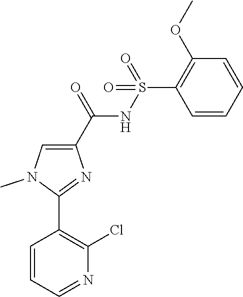 [SMILES: COc1ccccc1S(=O)(=O)NC(=O)c1cn(C)c(-c2cccnc2Cl)n1] | Example 492: ¹H-NMR(400.0 MHz, d₆-DMSO): δ = 8.62(1.68); 8.615(1.84); 8.608(1.85); 8.603(1.84); 8.18(3.84); 8.109(0.44); 8.091(1.65); 8.086(1.73); 8.072(1.85); 8.067(1.82); 7.902(1.62); 7.899 (1.76); 7.883(1.79); 7.879(1.86); 7.853(0.32); 7.849(0.33); 7.686(0.75); 7.683(0.75); 7.665(1.65); 7.647(1.16); 7.637(1.88); 7.625(1.84); 7.618 (1.68); 7.606(1.59); 7.24(2.21); 7.219(2.35); 7.198(0.36); 7.163(1.2); 7.144 (2.36); 7.125(1.41); 3.85(16); 3.838(3.08); 3.634(2.76); 3.554(14.92); 3.412 (0.43); 3.322(4.05); 2.671(0.65); 2.541(22.63); 2.506(80.27); 2.502(108.72); 2.498(85.41); 2.328(0.64); 2.074(1.28); 0(42.33) |
| 493 | 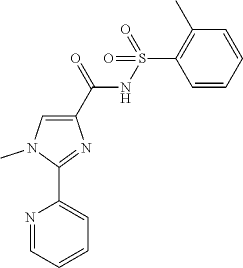 [SMILES: Cc1ccccc1S(=O)(=O)NC(=O)c1cn(C)c(-c2ccccn2)n1] | Example 493: ¹H-NMR(400.0 MHz, d₆-DMSO): δ = 8.655(1.56); 8.644(1.55); 8.23(1.86); 8.21(2.17); 8.075(3.91); 8.036(1.68); 8.018(1.75); 8.016(1.74); 7.977(0.95); 7.972(0.98); 7.957(1.66); 7.953 (1.64); 7.938(0.91); 7.934(0.89); 7.586(0.65); 7.57(1.5); 7.567(1.53); 7.551 (1.01); 7.548(1.01); 7.46(2.06); 7.45(1.26); 7.448(1.42); 7.441(2.49); 7.432 (1.2); 7.429(1.16); 7.421(0.75); 7.399(1.73); 7.38(1.41); 4.075(16); 3.382 (0.35); 3.378(0.35); 3.346(0.36); 3.336(0.36); 3.183(0.43); 2.675(0.39); 2.671(0.52); 2.666(0.42); 2.624(12.48); 2.51(29.31); 2.506(57.36); 2.502 (76.96); 2.497(57.83); 2.493(28.67); 2.329(0.44); 2.324(0.32); 2.074(0.67); 0(2.56) |
| 494 | 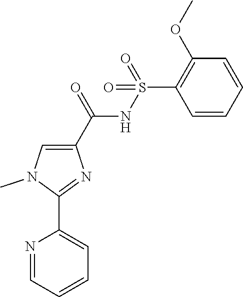 [SMILES: COc1ccccc1S(=O)(=O)NC(=O)c1cn(C)c(-c2ccccn2)n1] | Example 494: ¹H-NMR(400.0 MHz, d₆-DMSO): δ = 8.661(1.44); 8.65(1.48); 8.649(1.45); 8.229(1.77); 8.209(2.09); 8.12(4.42); 7.979(0.91); 7.975(0.94); 7.96(1.55); 7.955(1.61); 7.94(0.89); 7.936(0.92); 7.916(1.58); 7.912(1.73); 7.897(1.73); 7.892(1.78); 7.688(0.72); 7.684 (0.75); 7.666(1.31); 7.648(0.89); 7.644(0.86); 7.469(1.01); 7.466(1.08); 7.457(1.01); 7.454(1.15); 7.45(1.09); 7.447(1.03); 7.438(0.99); 7.435(1); 7.236(2); 7.215(1.81); 7.169(1.07); 7.151(1.95); 7.132(0.96); 4.084(14.96); 4.072(0.68); 3.855(16); 3.325(0.6); 3.322(0.59); 2.671(0.32); 2.524(1.04); 2.511(19.01); 2.506(38.28); 2.502(52.07); 2.497(39.29); 2.493(19.66); 2.329(0.32); 2.074(0.32); 1.522(1.17); 0(1.7) |
| 495 | 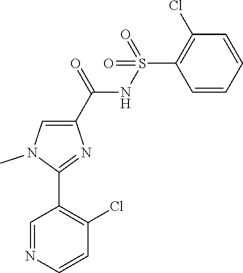 [SMILES: Cn1cc(C(=O)NS(=O)(=O)c2ccccc2Cl)nc1-c1cnccc1Cl] | Example 495: ¹H-NMR(400.0 MHz, d₆-DMSO): δ = 8.724(4.01); 8.712(2.13); 8.676(0.51); 8.664(0.43); 8.314(0.52); 8.209 (1.06); 8.152(1.61); 8.132(1.97); 7.995(0.58); 7.935(1.42); 7.79(2.72); 7.777(2.78); 7.684(1.03); 7.655(2.05); 7.636(0.85); 7.614(0.91); 7.595 (1.31); 7.578(0.65); 3.96(1.01); 3.827(1.11); 3.788(0.39); 3.742(0.52); 3.627 (1.3); 3.616(5.12); 3.567(16); 3.535(3.25); 3.398(3.18); 3.185(1.23); 2.671 (1.67); 2.506(206.14); 2.502(277.42); 2.498(213.59); 2.328(1.66); 2.074 (2.66); 1.512(4.25); 1.481(11.79); 0.146(0.83); 0.008(8.51); 0(187.22); −0.149(0.89) |
| 496 | 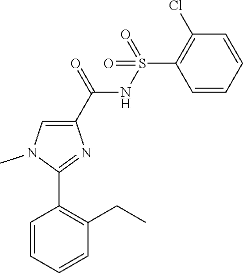 [SMILES: CCc1ccccc1-c1nc(C(=O)NS(=O)(=O)c2ccccc2Cl)cn1C] | Example 496: ¹H-NMR(400.0 MHz, d₆-DMSO): δ = 8.314(0.36); 8.132(2.29); 8.129(2.39); 8.112(5.8); 7.938(0.43); 7.873(2.04); 7.646(0.39); 7.629(1.37); 7.626(1.35); 7.609(4.2); 7.604(3.61); 7.589(1.1); 7.572(1.43); 7.568(1.14); 7.553(1.76); 7.536(0.92); 7.53(1.25); 7.526(0.97); 7.507(2.03); 7.492(1.55); 7.489(1.68); 7.453(0.68); 7.442(3.33); 7.421 (2.23); 7.408(1.36); 7.4(2.89); 7.368(1.84); 7.366(1.83); 7.348(2.21); 7.332 (0.96); 7.317(0.71); 7.313(0.72); 7.297(1.26); 7.292(0.89); 3.493(19.88); 3.456(1.34); 3.438(5.79); 2.675(0.42); 2.67(0.66); 2.666(0.43); 2.523(1.06); 2.51(59.75); 2.506(122.51); 2.501(170.14); 2.497(133.37); 2.474(7.47); 2.46(3.28); 2.441(1.49); 2.332(1.06); 2.328(1.31); 2.324(1.05); 2.073(0.39); 1.507(16); 1.023(5.61); 1.004(12.34); 0.999(4.78); 0.993(1.66); 0.985(5.77); 0.008(1.81); 0(56.75); −0.008(3.08) |
| 497 | 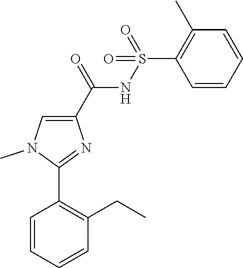 [SMILES: CCc1ccccc1-c1nc(C(=O)NS(=O)(=O)c2ccccc2C)cn1C] | Example 497: ¹H-NMR(600.1 MHz, CD3CN): δ = 8.118(1.26); 8.105(1.3); 7.731(3.62); 7.574(0.64); 7.572(0.66); 7.562(1.49); 7.56(1.49); 7.549(0.91); 7.548(0.89); 7.492(0.75); 7.49(0.78); 7.48(1.66); 7.477(1.66); 7.468(1.4); 7.465(1.45); 7.431(2.37); 7.43(2.39); 7.42(1.85); 7.41(0.75); 7.384(1.62); 7.371(1.41); 7.339(0.71); 7.338(0.69); 7.327(1.72); 7.325(1.67); 7.315(1.22); 7.313(1.2); 7.298(2.32); 7.296(2.4); 7.285(1.21); 7.283(1.09); 3.441(16); 2.63(11.71); 2.56(1.1); 2.547(3.34); 2.535(3.42); 2.522(1.23); 1.955(0.55); 1.95(0.56); 1.947(3.29); 1.943(5.8); 1.938(8.43); 1.934(5.77); 1.93(2.91); 1.073(3.33); 1.06(6.71); 1.047(3.35); 0(0.97) |
| 498 | 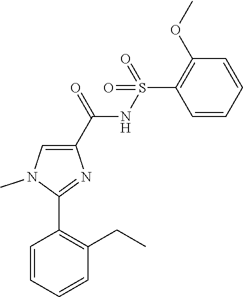 [SMILES: CCc1ccccc1-c1nc(C(=O)NS(=O)(=O)c2ccccc2OC)cn1C] | Example 498: ¹H-NMR(400.0 MHz, d₆-DMSO): δ = 8.1(5.57); 7.907(1.59); 7.903(1.73); 7.887(1.74); 7.883(1.78); 7.689(0.76); 7.685(0.78); 7.667(1.5); 7.65(0.93); 7.646(0.89); 7.497(0.47); 7.491(0.51); 7.476(1.23); 7.473(1.16); 7.461(1.13); 7.456(1.28); 7.427(2.39); 7.409 (1.22); 7.364(0.72); 7.35(2.85); 7.346(3.87); 7.328(1.44); 7.311(0.46); 7.244 (2.21); 7.223(2.02); 7.167(1.2); 7.148(2.17); 7.129(1.09); 3.842(16); 3.472 (14.63); 3.456(0.68); 2.67(0.33); 2.541(19.12); 2.527(3.4); 2.506(41.48); 2.501(55.22); 2.497(42.31); 2.328(0.35); 2.074(0.39); 1.023(3.84); 1.005 (8.27); 0.986(3.79); 0(4.69) |
| 499 | 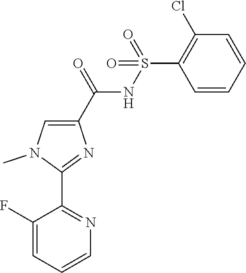 [SMILES: Cn1cc(C(=O)NS(=O)(=O)c2ccccc2Cl)nc1-c1ncccc1F] | Example 499: ¹H-NMR(400.0 MHz, d₆-DMSO): δ = 8.579(1.04); 8.576(1.83); 8.572(1.18); 8.568(1.16); 8.564(1.9); 8.561(1.14); 8.212(1.65); 8.163(1.44); 8.159(1.57); 8.143(1.59); 8.14(1.68); 8.134(0.5); 7.966(0.87); 7.964(0.94); 7.945(1.07); 7.941(1.52); 7.938(1.04); 7.919 (0.99); 7.916(1.05); 7.71(0.34); 7.692(0.98); 7.676(1.13); 7.672(1.17); 7.657 (3); 7.646(1.73); 7.641(1.03); 7.635(2.15); 7.624(2.15); 7.614(0.95); 7.604 (1.27); 7.587(0.57); 7.583(0.54); 3.827(16); 3.184(0.5); 2.524(0.51); 2.52 (0.79); 2.511(17.09); 2.506(38.2); 2.502(55.22); 2.497(41.87); 2.493(20.39); 2.328(0.33); 2.074(0.59); 0(2.65) |
| 500 | 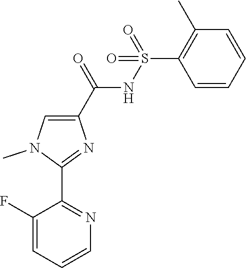 [SMILES: Cc1ccccc1S(=O)(=O)NC(=O)c1cn(C)c(-c2ncccc2F)n1] | Example 500: ¹H-NMR(400.0 MHz, d₆-DMSO): δ = 8.575(2.06); 8.564(2.07); 8.167(5.49); 8.036(2.16); 8.016(2.32); 7.968 (1.15); 7.944(1.86); 7.921(1.33); 7.657(0.9); 7.647(1.66); 7.636(1.61); 7.626 (1.4); 7.615(0.73); 7.592(0.89); 7.573(2.16); 7.554(1.42); 7.46(1.29); 7.441 (2.09); 7.422(0.96); 7.403(2.31); 7.384(1.89); 3.812(16); 2.671(0.36); 2.613 (14.36); 2.506(45.61); 2.502(54.06); 2.329(0.32); 2.074(0.37); 0(1.56) |
| 501 | 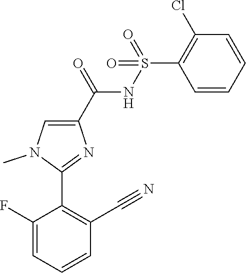 [SMILES: Cn1cc(C(=O)NS(=O)(=O)c2ccccc2Cl)nc1-c1c(F)cccc1C#N] | Example 501: ¹H-NMR(400.0 MHz, d₆-DMSO): δ = 8.314(0.35); 8.293(0.71); 8.172(0.44); 8.152(0.48); 8.05(2.05); 7.972(0.38); 7.963(0.37); 7.952(0.9); 7.941(1.1); 7.931(0.75); 7.863(0.68); 7.85(1.76); 7.839(1.48); 7.821(1.01); 7.71(0.35); 7.692(0.41); 7.673(0.67); 7.653(0.33); 7.634(0.34); 7.616(0.44); 3.623(2.67); 3.589(5.16); 3.44(0.47); 3.329(4.37); 3.186(0.45); 2.67(1.25); 2.501(218.41); 2.328(1.31); 2.073(1.56); 1.521(16); 1.486(0.4); 1.157(0.35); 0(4.54) |
| 502 | 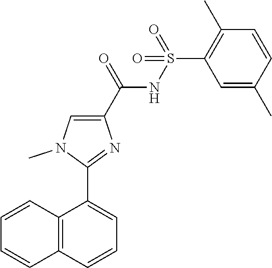 [SMILES: Cc1ccc(C)c(S(=O)(=O)NC(=O)c2cn(C)c(-c3cccc4ccccc34)n2)c1] | Example 502: : ¹H-NMR(400.0 MHz, d₆-DMSO): δ = 8.180 (6.9); 8.133 (3.4); 8.113 (2.4); 8.059 (2.3); 8.040 (2.4); 7.846 (4.0); 7.694 (3.1); 7.680 (4.1); 7.676 (4.5); 7.666 (4.4); 7.646 (3.4); 7.628 (1.5); 7.623 (0.9); 7.620 (1.0); 7.606 (2.2); 7.603 (2.0); 7.586 (3.4); 7.582 (3.3); 7.565 (1.8); 7.562 (1.8); 7.548 (0.7); 7.544 (0.6); 7.387 (1.6); 7.368 (2.3); 7.286 (3.4); 7.267 (2.4); 3.885 (0.4); 3.718 (0.4); 3.674 (0.5); 3.610 (0.6); 3.499 (21.4); 3.448 (0.8); 3.381 (0.7); 3.336 (0.6); 3.320 (0.6); 3.184 (0.4); 2.762 (0.6); 2.675 (0.7); 2.671 (1.0); 2.666 (0.8); 2.568 (16.0); 2.524 (2.6); 2.506 (115.1); 2.502 (159.9); 2.497 (124.5); 2.371 (15.5); 2.333 (0.7); 2.328 (1.0); 2.324 (0.8); 2.074 (2.2); 0.146(0.8); 0.008 (6.4); 0.000 (165.8); −0.150 (0.8) |
| 503 | 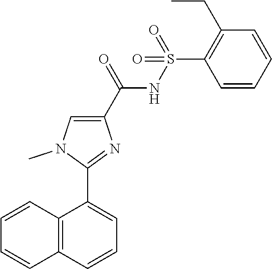 [SMILES: CCc1ccccc1S(=O)(=O)NC(=O)c1cn(C)c(-c2cccc3ccccc23)n1] | Example 503: ¹H-NMR(400.0 MHz, d₆-DMSO): δ = 8.174(4.63); 8.135(1.64); 8.115(1.74); 8.06(1.72); 8.04(3.56); 8.022(1.88); 8.019(2.02); 7.7(0.86); 7.696(1.21); 7.682(3.96); 7.679(3.69); 7.667(2.93); 7.647(2.58); 7.629(1.79); 7.62(0.97); 7.607(2.81); 7.588(2.18); 7.582(2.28); 7.577(1.75); 7.56(1.42); 7.558(1.31); 7.544(0.57); 7.54(0.51); 7.456(2.21); 7.445(1.36); 7.437(1.84); 7.426(1.88); 7.407(0.86); 3.503(16); 3.1(1.05); 3.081(3.33); 3.062(3.41); 3.044(1.13); 2.671(0.4); 2.524(0.88); 2.51(23.89); 2.506(50.99); 2.501(72.33); 2.497(55.62); 2.493(27.88); 2.333(0.34); 2.328 (0.44); 2.324(0.34); 2.074(13.42); 1.208(4.03); 1.189(8.89); 1.171(4.03); 0.146(0.36); 0.008(2.67); 0(82.32); −0.008(3.48); −0.15(0.38) |
| 504 | 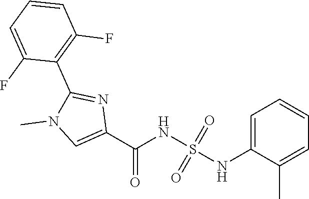 [SMILES: Cc1ccccc1NS(=O)(=O)NC(=O)c1cn(C)c(-c2c(F)cccc2F)n1] | Example 504: ¹H-NMR(300.1 MHz, d₆-DMSO): δ = 11.23(0.54); 9.652(2.51); 8.219(4.78); 7.747(0.39); 7.718(0.95); 7.696 (1.73); 7.668(1.09); 7.646(0.53); 7.354(2.99); 7.326(4.46); 7.299(2.57); 7.252(1.13); 7.245(1.38); 7.221(2.95); 7.203(1.83); 7.193(2); 7.17(0.97); 7.154(2.24); 7.144(2.81); 7.133(3.46); 7.121(2.11); 3.68(1.01); 3.567 (14.09); 3.547(0.64); 3.416(0.34); 3.326(4.36); 2.726(0.82); 2.579(0.33); 2.507(90.25); 2.501(114.76); 2.495(80.36); 2.28(16); 1.908(0.6); 1.236 (0.68); 0.195(0.33); 0.011(4.14); 0(74.22); −0.011(2.91); −0.199(0.33) |
| 505 | 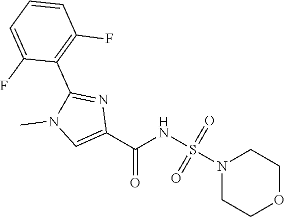 [SMILES: Cn1cc(C(=O)NS(=O)(=O)N2CCOCC2)nc1-c1c(F)cccc1F] | Example 505: ¹H-NMR(300.1 MHz, d₆-DMSO): δ = 8.17(2.71); 8.136(1.08); 7.754(0.51); 7.731(1.1); 7.704(1.89); 7.678(1.23); 7.654(0.53); 7.364(3.26); 7.338(5.14); 7.31(2.7); 6.531(0.32); 3.642(5.96); 3.627(8.12); 3.612(6.3); 3.579(16); 3.535(0.88); 3.327(16.54); 3.245(6.34); 3.231(7.44); 3.11(0.33); 3.062(0.57); 2.891(0.38); 2.728(0.88); 2.502 (50.83); 1.446(0.33); 1.237(0.43); 1.212(0.37); 0(31.12) |
| 506 | 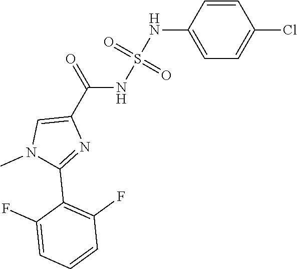 [SMILES: Cn1cc(C(=O)NS(=O)(=O)Nc2ccc(Cl)cc2)nc1-c1c(F)cccc1F] | Example 506: ¹H-NMR(300.1 MHz, d₆-DMSO): δ = 10.688(3.2); 8.179(7.79); 7.748(0.44); 7.725(1.02); 7.719(0.95); 7.697 (1.82); 7.674(1.01); 7.669(1.13); 7.646(0.5); 7.359(5.45); 7.351(4.76); 7.33 (9.42); 7.324(6.29); 7.297(2.69); 7.254(1.26); 7.245(7.73); 7.238(2.27); 7.215(4.61); 3.546(16); 3.334(0.33); 3.306(0.35); 2.508(17.56); 2.502 (22.34); 2.497(15.75); 1.236(0.47); 0(14.07) |
| 507 | 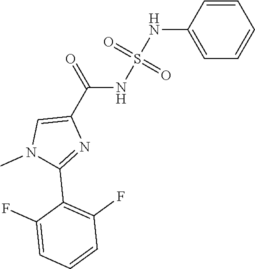 [SMILES: Cn1cc(C(=O)NS(=O)(=O)Nc2ccccc2)nc1-c1c(F)cccc1F] | Example 507: ¹H-NMR(300.1 MHz, d₆-DMSO): δ = 11.623(0.4); 8.13(0.98); 7.738(0.43); 7.715(1); 7.709(0.93); 7.687(1.85); 7.659(1.17); 7.636(0.5); 7.342(3.39); 7.315(5.18); 7.288(3.82); 7.258(3.88); 7.235(4.63); 7.218(6.5); 7.194(2.25); 7.034(1.1); 7.011(1.63); 6.988(0.79); 3.532(16); 3.327(0.93); 2.508(30.39); 2.503(39.47); 2.497(28.34); 1.91 (2.45); 1.235(0.46); 0.01(1.35); 0(26.39); −0.011(1.17) |
| 508 | 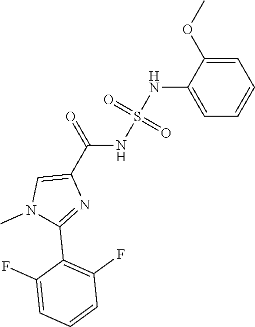 [SMILES: COc1ccccc1NS(=O)(=O)NC(=O)c1cn(C)c(-c2c(F)cccc2F)n1] | Example 508: ¹H-NMR(300.1 MHz, d₆-DMSO): δ = 9.161(1.85); 8.198(3.96); 7.753(0.32); 7.73(0.71); 7.725(0.65); 7.702(1.36); 7.679(0.73); 7.674(0.86); 7.652(0.35); 7.359(2.45); 7.333(5.1); 7.306(3.35); 7.191(0.59); 7.186(0.58); 7.163(1.34); 7.139(1.02); 7.134(0.95); 7.028 (2.12); 7.004(1.44); 6.94(1.07); 6.936(1.02); 6.914(1.71); 6.89(0.79); 3.798 (0.35); 3.68(16); 3.566(11.44); 3.168(0.39); 2.507(15.13); 2.501(19.52); 2.496(13.97); 1.909(4.84); 0.011(0.66); 0(13.12) |
| 509 | 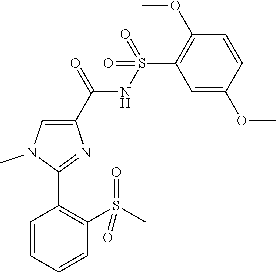 | Example 509: ¹H-NMR(400.0 MHz, d₆-DMSO): δ = 8.118(0.96); 8.107(0.64); 8.102(0.71); 8.099(0.94); 8.095(0.84); 7.922(2.3); 7.891(0.4); 7.888(0.44); 7.88(0.73); 7.876(0.81); 7.869(0.65); 7.862(0.74); 7.858(0.64); 7.851(0.89); 7.847(0.94); 7.832(0.77); 7.828(0.77); 7.709 (0.39); 7.706(0.41); 7.688(0.35); 7.674(0.7); 7.671(0.74); 7.656(0.58); 7.653 (0.59); 7.386(0.69); 7.378(0.77); 7.182(0.39); 5.754(3.26); 3.784(4.37); 3.78 (3.76); 3.423(6.14); 3.415(3.68); 3.371(6.23); 3.354(3.48); 3.324(0.4); 2.541 (2.9); 2.506(12.46); 2.501(17.32); 2.497(13.55); 1.506(16); 0(4.45) |
| 510 | 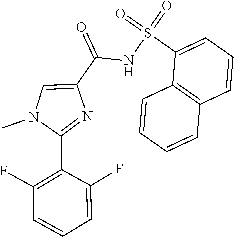 [SMILES: Cn1cc(C(=O)NS(=O)(=O)c2cccc3ccccc23)nc1-c1c(F)cccc1F] | Example 510: ¹H-NMR(400.0 MHz, d₆-DMSO): δ = 8.799(2.04); 8.779(2.11); 8.369(1.4); 8.353(1.49); 8.315(0.95); 8.298(1.07); 8.275(1.16); 8.109(1.84); 8.089(2.99); 7.739(1.19); 7.717(2.66); 7.695 (3.15); 7.674(2.28); 7.666(2.28); 7.644(1.69); 7.628(0.69); 7.34(2.67); 7.32 (4.54); 7.299(2.34); 3.522(16); 3.458(0.32); 3.316(1.54); 3.204(0.38); 2.67 (2.18); 2.506(277.39); 2.501(360.1); 2.497(274.36); 2.328(2.08); 2.074 (0.82); 0(59.86) |
| 511 | 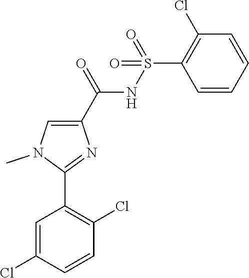 [SMILES: Cn1cc(C(=O)NS(=O)(=O)c2ccccc2Cl)nc1-c1cc(Cl)ccc1Cl] | Example 511: ¹H-NMR(400.0 MHz, d₆-DMSO): δ = 8.174(3.11); 8.152(1.98); 8.133(2.12); 7.713(3.9); 7.69(9.59); 7.668(2.16); 7.656(2.98); 7.639(1.03); 7.617(1.15); 7.614(1.16); 7.597(1.72); 7.58(0.73); 7.577(0.75); 3.542(16); 3.185(0.5); 2.671(0.33); 2.506(45.7); 2.502(62.06); 2.498(48.98); 2.328(0.43); 2.074(0.43); 1.507(0.34); 0.008(0.78); 0(19.48) |
| 512 | 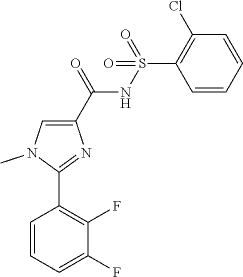 [SMILES: Cn1cc(C(=O)NS(=O)(=O)c2ccccc2Cl)nc1-c1cccc(F)c1F] | Example 512: ¹H-NMR(400.0 MHz, d₆-DMSO): δ = 8.314(0.52); 8.199(4.22); 8.157(2.72); 8.154(2.87); 8.137(3.21); 8.134 (3.54); 7.709(0.65); 7.69(2.47); 7.671(3.17); 7.658(4.63); 7.64(2.84); 7.621 (2.36); 7.601(2.52); 7.584(1.14); 7.468(1.03); 7.448(2.26); 7.433(1.8); 7.408 (1.32); 7.396(1.28); 7.388(1.62); 7.376(1.58); 7.356(0.62); 3.638(15.77); 3.635(16); 2.675(0.77); 2.671(1.07); 2.666(0.79); 2.506(136.21); 2.502 (187.95); 2.497(144.85); 2.333(0.89); 2.328(1.17); 2.324(0.91); 2.074(7.69); 0.008(2.31); 0(60.67); −0.008(2.75) |
| 513 | 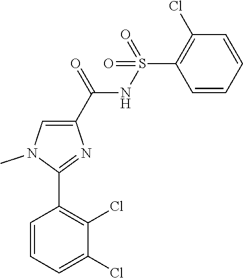 [SMILES: Cn1cc(C(=O)NS(=O)(=O)c2ccccc2Cl)nc1-c1cccc(Cl)c1Cl] | Example 513: ¹H-NMR(400.0 MHz, d₆-DMSO): δ = 8.183(2.62); 8.154(1.86); 8.151(1.87); 8.133(2.85); 7.875(1.79); 7.87(1.86); 7.856(2.05); 7.851(2.01); 7.704(0.43); 7.686(1.35); 7.667(1.75); 7.655(2.72); 7.638(0.99); 7.616(1.14); 7.596(1.66); 7.577(1.77); 7.562(3.06); 7.558(2.65); 7.55(2.97); 7.53(3.09); 7.511(1.06); 3.521(16); 2.671(0.61); 2.506(71.8); 2.502(94.25); 2.497(71.74); 2.329(0.53); 2.074(3.24); 1.508(0.37); 0(29.68); −0.008(1.25) |
| 514 | 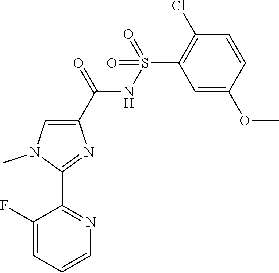 | Example 514: ¹H-NMR(400.0 MHz, d₆-DMSO): δ = 8.579(1.71); 8.571(1.18); 8.568(1.74); 8.236(3.94); 7.971(0.84); 7.969 (0.82); 7.95(1.08); 7.946(1.46); 7.924(0.96); 7.921(0.93); 7.662(0.86); 7.652 (1.44); 7.641(1.52); 7.63(1.21); 7.619(0.93); 7.614(2.98); 7.606(3.08); 7.575 (2.49); 7.553(2.93); 7.295(1.47); 7.288(1.42); 7.273(1.27); 7.265(1.22); 3.857(16); 3.832(13.72); 2.507(25.21); 2.502(34.45); 2.498(26.34); 2.074 (0.42); 0.008(0.5); 0(12.18) |
| 515 | 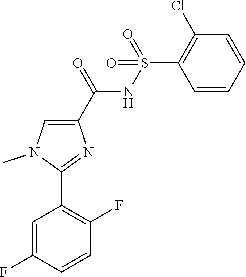 [SMILES: Cn1cc(C(=O)NS(=O)(=O)c2ccccc2Cl)nc1-c1cc(F)ccc1F] | Example 515: ¹H-NMR(601.6 MHz, d₆-DMSO): δ = 19.959(0.75); 8.176(3.31); 8.15(2.71); 8.137(2.83); 7.7(0.68); 7.688(1.91); 7.675(1.78); 7.654(3.48); 7.641(1.72); 7.612(1.49); 7.6(2.45); 7.586(1.19); 7.522(1.53); 7.492(2.3); 7.484(3.89); 7.474(2.62); 3.624(16); 2.612(0.69); 2.518(1.42); 2.515(1.42); 2.504(109.6); 2.501(153.39); 2.498(115.71); 2.384 (0.82); 2.071(1.67); 0(25.37) |
| 516 | 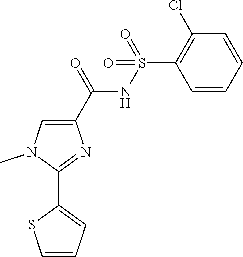 [SMILES: Cn1cc(C(=O)NS(=O)(=O)c2ccccc2Cl)nc1-c1cccs1] | Example 516: ¹H-NMR(601.6 MHz, d₆-DMSO): δ = 8.155(1.59); 8.153(1.75); 8.142(1.7); 8.14(1.78); 8.118(3.68); 7.73(2.08); 7.722(2.11); 7.706(0.53); 7.704(0.55); 7.691(1.43); 7.681(1.24); 7.679 (1.27); 7.655(2.5); 7.644(1.21); 7.617(1.1); 7.605(1.78); 7.595(2.54); 7.59 (2.57); 7.211(1.81); 7.205(1.87); 7.203(2.01); 7.197(1.72); 3.869(16); 3.749 (0.34); 3.712(0.34); 3.555(0.53); 2.504(29.97); 2.501(42.68); 2.498(32.69); 2.071(2.27); 0(8.2) |
| 517 | 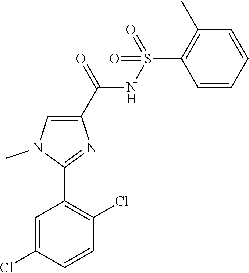 [SMILES: Cc1ccccc1S(=O)(=O)NC(=O)c1cn(C)c(-c2cc(Cl)ccc2Cl)n1] | Example 517: ¹H-NMR(601.6 MHz, d₆-DMSO): δ = 8.112(5.01); 8.025(1.78); 8.012(1.84); 7.701(1.99); 7.697(3.61); 7.684 (4.63); 7.68(3.89); 7.676(2.37); 7.666(0.53); 7.662(0.56); 7.576(0.79); 7.563(1.78); 7.551(1.09); 7.445(1.02); 7.432(1.76); 7.42(0.83); 7.395(1.83); 7.382(1.6); 3.529(16); 2.609(13.26); 2.504(19.86); 2.501(28.21); 2.498 (21.53); 2.071(2.61); 0(10.75) |
| 518 | 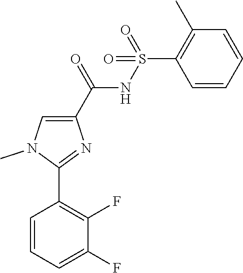 [SMILES: Cc1ccccc1S(=O)(=O)NC(=O)c1cn(C)c(-c2cccc(F)c2F)n1] | Example 518: ¹H-NMR(601.6 MHz, d₆-DMSO): δ = 8.14(6.01); 8.028(2.28); 8.015(2.39); 7.668(0.45); 7.653(1.11); 7.639(1.12); 7.624(0.49); 7.578(1.01); 7.566(2.33); 7.554(1.4); 7.456(0.84); 7.446(2.56); 7.434(3.43); 7.422(1.12); 7.396(2.8); 7.384(2.91); 7.364(0.51); 3.624 (13.18); 2.612(16); 2.502(20.82); 2.072(0.71); 0(6.75) |
| 519 | 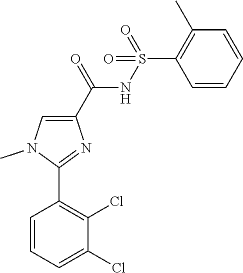 [SMILES: Cc1ccccc1S(=O)(=O)NC(=O)c1cn(C)c(-c2cccc(Cl)c2Cl)n1] | Example 519: ¹H-NMR(400.0 MHz, d₆-DMSO): δ = 8.114(3.27); 8.023(1.74); 8.003(1.83); 7.868(1.67); 7.863(1.86); 7.85(1.87); 7.844(2); 7.579(0.7); 7.57(0.73); 7.564(2.14); 7.56(1.9); 7.551(3.45); 7.544 (5.57); 7.525(2.93); 7.506(0.91); 7.448(1.02); 7.429(1.65); 7.41(0.79); 7.394 (1.81); 7.376(1.47); 3.506(16); 3.326(0.33); 2.675(0.46); 2.67(0.63); 2.666 (0.5); 2.605(13.32); 2.51(34.51); 2.506(68.81); 2.501(91.96); 2.497(69.76); 2.333(0.39); 2.328(0.54); 2.324(0.42); 2.073(4.71); 0.008(1.03); 0(19.84); −0.008(1.16) |
| 520 | 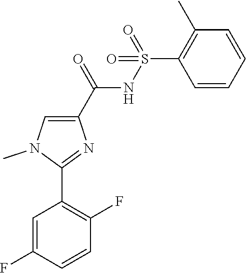 [SMILES: Cc1ccccc1S(=O)(=O)NC(=O)c1cn(C)c(-c2cc(F)ccc2F)n1] | Example 520: ¹H-NMR(400.0 MHz, d₆-DMSO): δ = 8.125(7.71); 8.029(2.01); 8.027(2.18); 8.01(2.2); 8.007(2.26); 7.59(0.84); 7.586(0.92); 7.571(2.08); 7.568(2.17); 7.552(1.45); 7.549(1.54); 7.542 (0.96); 7.537(0.77); 7.532(0.67); 7.523(1.23); 7.518(0.93); 7.516(0.94); 7.511(0.91); 7.503(2.77); 7.499(1.95); 7.487(3.16); 7.471(2.28); 7.466 (1.79); 7.455(1.41); 7.435(2.09); 7.416(0.96); 7.4(2.33); 7.381(1.91); 3.614 (10.99); 3.611(11.25); 2.609(16); 2.524(0.51); 2.511(12.51); 2.506(25.61); 2.502(34.09); 2.497(25); 2.493(12.57); 2.075(5.69); 0(3.13) |
| 521 | 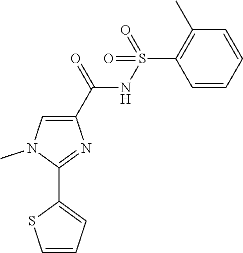 [SMILES: Cc1ccccc1S(=O)(=O)NC(=O)c1cn(C)c(-c2cccs2)n1] | Example 521: ¹H-NMR(400.0 MHz, d₆-DMSO): δ = 8.058(5.74); 8.027(1.49); 8.024(1.68); 8.007(1.64); 8.004(1.78); 7.726 (2.02); 7.724(2.39); 7.714(2.21); 7.711(2.42); 7.584(2.71); 7.581(2.53); 7.575(2.5); 7.572(2.89); 7.566(1.88); 7.551(1.07); 7.548(1.11); 7.455(1); 7.435(1.59); 7.416(0.71); 7.399(1.75); 7.38(1.43); 7.212(2.23); 7.203(2.13); 7.199(2.32); 7.19(2.06); 3.858(16); 2.612(12.39); 2.524(0.45); 2.519(0.77); 2.51(13.77); 2.506(29.63); 2.501(40.15); 2.497(29.77); 2.492(14.87); 2.074 (0.93); 0(3.6) |
| 522 | 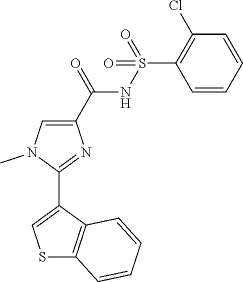 [SMILES: Cn1cc(C(=O)NS(=O)(=O)c2ccccc2Cl)nc1-c1csc2ccccc12] | Example 522: ¹H-NMR(400.0 MHz, d₆-DMSO): δ = 8.317(1.14); 8.311(0.92); 8.305(0.82); 8.294(1.29); 8.278(6.39); 8.188 (3.96); 8.176(1.72); 8.173(1.82); 8.156(1.72); 8.153(1.82); 8.108(1.38); 8.104(1.21); 8.098(0.95); 8.094(0.96); 8.091(1.11); 8.086(1.46); 7.717 (0.34); 7.713(0.37); 7.697(1.17); 7.693(1.12); 7.68(1.63); 7.676(1.8); 7.671 (2.19); 7.667(2.76); 7.652(0.96); 7.631(1.24); 7.627(0.99); 7.611(1.45); 7.607(1.29); 7.594(0.77); 7.59(0.67); 7.506(0.52); 7.494(1.82); 7.489(3); 7.48(3.16); 7.47(2.81); 7.466(1.72); 7.454(0.45); 3.804(16); 2.67(0.43); 2.524(0.93); 2.519(1.43); 2.51(24.97); 2.506(52.88); 2.501(71.16); 2.497 (51.89); 2.492(25.57); 2.328(0.43); 2.324(0.33); 2.074(0.67); 0.008(1.8); 0(60.59); −0.008(2.41) |
| 523 | 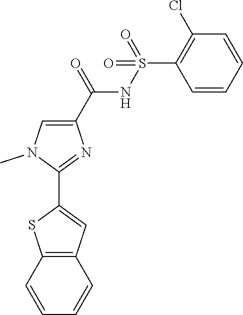 [SMILES: Cn1cc(C(=O)NS(=O)(=O)c2ccccc2Cl)nc1-c1cc2ccccc2s1] | Example 523: ¹H-NMR(400.0 MHz, d₆-DMSO): δ = 8.213(3.25); 8.176(1.62); 8.172(1.83); 8.156(1.8); 8.152(1.92); 8.029(1.25); 8.014(1.22); 8.006(1.37); 7.942(5.1); 7.936(1.77); 7.926(1.37); 7.918(1.05); 7.913(1.47); 7.733(0.4); 7.729(0.46); 7.712(1.23); 7.695(1.43); 7.691(1.49); 7.678(2); 7.674(2.67); 7.658(1.07); 7.654(0.89); 7.643(1.17); 7.639(1.04); 7.623(1.53); 7.605(0.73); 7.601(0.7); 7.454(0.47); 7.442(3.15); 7.436(1.94); 7.432(2.12); 7.429(2.14); 7.424(1.85); 7.419(3.09); 7.406(0.44); 3.998(16); 3.354(0.37); 2.675(0.73); 2.67(1.03); 2.666(0.78); 2.523(2.91); 2.51(61.54); 2.506(127.09); 2.501(170.32); 2.497(125.99); 2.492(63.76); 2.332(0.76); 2.328(1.05); 2.323(0.8); 2.074(1.16); 1.523(0.34); 0.146(0.49); 0.008(4.39); 0(112.39); −0.008(5.12); −0.15(0.52) |
| 524 | 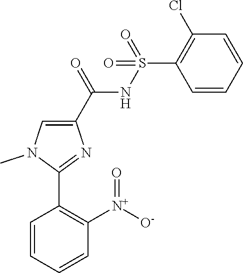 [SMILES: Cn1cc(C(=O)NS(=O)(=O)c2ccccc2Cl)nc1-c1ccccc1[N+](=O)[O-]] | Example 524: ¹H-NMR(400.0 MHz, d₆-DMSO): δ = 8.237(1.83); 8.234(1.87); 8.217(2.1); 8.214(2.12); 8.199(5.51); 8.144(1.76); 8.141(1.9); 8.125(1.93); 8.121(1.94); 7.936(0.66); 7.92(1.85); 7.917(1.9); 7.901(1.6); 7.898(1.54); 7.872(1.22); 7.868(1.47); 7.849(1.78); 7.833(0.79); 7.829(0.79); 7.816(2.25); 7.813(2.16); 7.798(1.63); 7.794(1.56); 7.706 (0.48); 7.702(0.5); 7.685(1.44); 7.668(1.85); 7.664(1.82); 7.656(2.46); 7.652(3); 7.636(1.11); 7.613(1.24); 7.609(1.12); 7.593(1.72); 7.576(0.86); 7.572(0.73); 7.248(0.83); 7.12(0.92); 6.992(0.86); 3.567(16); 3.53(0.46); 2.67(0.85); 2.538(0.35); 2.506(112.32); 2.501(141.6); 2.497(103.92); 2.328 (0.85); 2.074(1.48); 1.499(0.51); 0.146(0.36); 0.008(3.71); 0(79.04); −0.15 (0.34) |
| 525 | 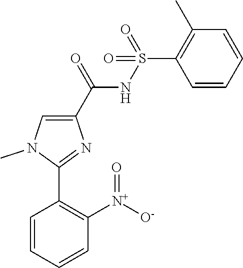 [SMILES: Cc1ccccc1S(=O)(=O)NC(=O)c1cn(C)c(-c2ccccc2[N+](=O)[O-])n1] | Example 525: ¹H-NMR(400.0 MHz, d₆-DMSO): δ = 8.225(1.67); 8.222(1.9); 8.205(1.96); 8.202(2.13); 8.132(5.74); 8.014(1.81); 7.994(1.9); 7.929(0.59); 7.926(0.71); 7.911(1.75); 7.908(1.95); 7.892(1.48); 7.889(1.55); 7.862(1.16); 7.858(1.5); 7.842(1.47); 7.838(1.83); 7.823(0.74); 7.819(0.76); 7.797(2.02); 7.794(2.07); 7.778(1.53); 7.775(1.52); 7.58(0.75); 7.564(1.63); 7.562(1.79); 7.546(1.07); 7.543(1.16); 7.448(1.06); 7.428 (1.73); 7.41(0.8); 7.395(1.89); 7.376(1.55); 3.549(16); 2.67(0.43); 2.585 (13.09); 2.523(0.84); 2.51(24.32); 2.505(50.62); 2.501(67.98); 2.496(51.02); 2.332(0.33); 2.328(0.42); 2.323(0.33); 2.074(0.42); 0.008(1.62); 0(44.52); −0.008(2.14) |
| 526 | 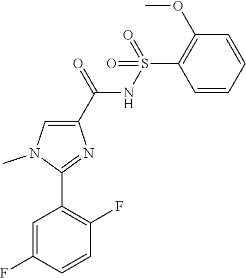 [SMILES: COc1ccccc1S(=O)(=O)NC(=O)c1cn(C)c(-c2cc(F)ccc2F)n1] | Example 526: ¹H-NMR(400.0 MHz, d₆-DMSO): δ = 9.807(0.36); 8.154(1.65); 7.899(1.63); 7.895(1.81); 7.88(1.84); 7.876(1.84); 7.679(0.64); 7.661(1.26); 7.642(0.76); 7.54(0.74); 7.524(1.15); 7.503(2.42); 7.486(3.09); 7.471(2.12); 7.236(1.76); 7.215(1.64); 7.159(1.01); 7.14(1.79); 7.121(0.91); 3.851(16); 3.622(10.13); 3.619(10.18); 3.317(10.17); 2.67 (2.18); 2.666(1.71); 2.523(5.16); 2.505(280.47); 2.501(366.77); 2.496 (279.44); 2.332(1.69); 2.328(2.22); 2.323(1.74); 0.146(0.91); 0.008(7.36); 0(200.84); −0.15(0.89) |
| 527 | 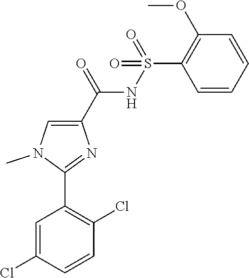 [SMILES: COc1ccccc1S(=O)(=O)NC(=O)c1cn(C)c(-c2cc(Cl)ccc2Cl)n1] | Example 527: ¹H-NMR(400.0 MHz, d₆-DMSO): δ = 8.159(3.29); 7.901(1.53); 7.897(1.72); 7.882(1.67); 7.878(1.79); 7.715 (0.66); 7.705(2.6); 7.701(3.5); 7.693(5.15); 7.691(4.56); 7.689(5.29); 7.684 (3.41); 7.666(1.65); 7.662(1.55); 7.647(0.81); 7.643(0.81); 7.241(1.98); 7.22(1.8); 7.163(1.06); 7.144(1.94); 7.125(0.94); 3.848(16); 3.534(14.66); 3.323(1.5); 2.67(0.48); 2.666(0.36); 2.524(1.15); 2.51(29.92); 2.506(61.62); 2.501(82.11); 2.497(61.24); 2.332(0.37); 2.328(0.49); 2.324(0.39); 0.008 (0.52); 0(14.53) |
| 528 | 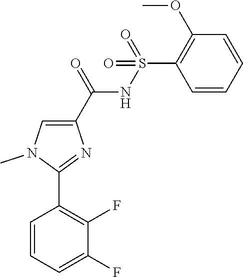 [SMILES: COc1ccccc1S(=O)(=O)NC(=O)c1cn(C)c(-c2cccc(F)c2F)n1] | Example 528: ¹H-NMR(400.0 MHz, d₆-DMSO): δ = 8.178(2.26); 7.903(1.49); 7.899(1.65); 7.883(1.67); 7.879(1.71); 7.684 (0.87); 7.668(1.58); 7.664(1.97); 7.647(1.05); 7.643(1.42); 7.622(0.38); 7.617(0.38); 7.47(0.54); 7.454(0.91); 7.451(1.2); 7.436(0.94); 7.41(0.68); 7.398(0.65); 7.393(0.8); 7.39(0.84); 7.378(0.8); 7.37(0.35); 7.239(1.76); 7.218(1.6); 7.162(0.95); 7.144(1.75); 7.125(0.86); 3.854(16); 3.636(8.25); 3.633(8.45); 3.321(1.86); 2.675(0.43); 2.67(0.61); 2.666(0.45); 2.524(1.34); 2.519(2.04); 2.51(36.12); 2.506(76.95); 2.501(103.27); 2.497(74.1); 2.492 (35.61); 2.332(0.44); 2.328(0.63); 2.324(0.46); 0.008(0.67); 0(23.48); −0.008(0.84) |
| 529 | 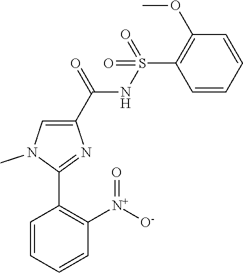 [SMILES: COc1ccccc1S(=O)(=O)NC(=O)c1cn(C)c(-c2ccccc2[N+](=O)[O-])n1] | Example 529: ¹H-NMR(400.0 MHz, d₆-DMSO): δ = 8.217(1.59); 8.214(1.69); 8.197(1.86); 8.194(1.9); 8.172(1.72); 7.93(0.63); 7.927(0.67); 7.911(1.78); 7.908(1.84); 7.889(2.72); 7.87(1.61); 7.866(1.71); 7.861(1.24); 7.857(1.43); 7.841(1.36); 7.837(1.64); 7.822(0.71); 7.818 (0.72); 7.807(1.94); 7.804(1.75); 7.788(1.37); 7.785(1.25); 7.679(0.53); 7.66 (0.99); 7.642(0.59); 7.24(1.42); 7.22(1.3); 7.158(0.82); 7.139(1.49); 7.12 (0.74); 3.832(15.75); 3.565(16); 3.32(13.24); 2.675(1.1); 2.67(1.55); 2.666 (1.15); 2.523(3.57); 2.519(5.45); 2.51(91.78); 2.506(195.71); 2.501(263.51); 2.496(189.22); 2.492(90.82); 2.337(0.53); 2.332(1.12); 2.328(1.57); 2.323 (1.17); 1.498(0.37); 0.008(1.57); 0(54.9); −0.008(2.04) |
| 530 | 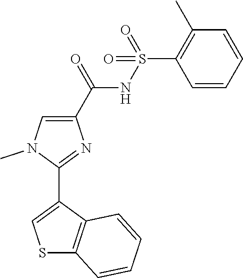 [SMILES: Cc1ccccc1S(=O)(=O)NC(=O)c1cn(C)c(-c2csc3ccccc23)n1] | Example 530: ¹H-NMR(400.0 MHz, d₆-DMSO): δ = 8.348(1.21); 8.343(1.25); 8.334(0.82); 8.328(1.14); 8.326(1.37); 8.251 (6.72); 8.132(5.12); 8.102(1.45); 8.1(1.36); 8.094(0.99); 8.086(1.37); 8.081 (1.43); 8.079(1.41); 8.049(1.49); 8.046(1.66); 8.029(1.66); 8.026(1.73); 7.595(0.6); 7.592(0.66); 7.576(1.51); 7.573(1.61); 7.557(1.04); 7.554(1.05); 7.514(0.33); 7.51(0.58); 7.496(1.69); 7.492(1.74); 7.489(1.79); 7.481(3.39); 7.473(1.95); 7.47(2.11); 7.465(2.48); 7.447(1.78); 7.427(0.7); 7.41(1.71); 7.392(1.41); 3.794(16); 2.67(0.43); 2.638(12.4); 2.524(0.85); 2.519(1.31); 2.51(23.15); 2.506(49.97); 2.501(67.78); 2.497(49.24); 2.492(24.08); 2.328 (0.42); 2.324(0.32); 2.074(1.96); 0.008(0.48); 0(16.72); −0.008(0.65) |
| 531 | 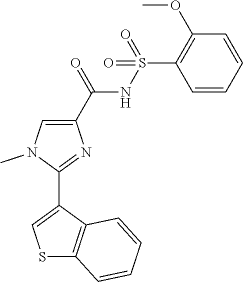 [SMILES: COc1ccccc1S(=O)(=O)NC(=O)c1cn(C)c(-c2csc3ccccc23)n1] | Example 531: ¹H-NMR(400.0 MHz, d₆-DMSO): δ = 8.374(1.02); 8.37(1.16); 8.352(1.2); 8.266(4.39); 8.162(3.46); 8.11(1.21); 8.093(1.3); 8.089(1.2); 7.928(1.2); 7.924(1.28); 7.908(1.31); 7.904(1.33); 7.699(0.56); 7.695(0.57); 7.678(1.11); 7.66(0.67); 7.656(0.63); 7.524(0.42); 7.51(1.16); 7.506(1.12); 7.492(1.97); 7.478(1.01); 7.474(1.05); 7.46(0.4); 7.256(1.65); 7.235(1.48); 7.18(0.92); 7.161(1.62); 7.142(0.84); 3.88(12.09); 3.816(11.1); 2.506(38); 2.501(48.94); 2.497(35.48); 2.074(16); 0(7.09) |
| 532 | 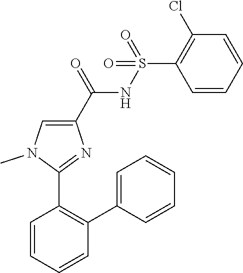 [SMILES: Cn1cc(C(=O)NS(=O)(=O)c2ccccc2Cl)nc1-c1ccccc1-c1ccccc1] | Example 532: ¹H-NMR(400.0 MHz, d₆-DMSO): δ = 8.118(2.26); 8.115(2.44); 8.097(2.72); 7.887(3.49); 7.718(0.86); 7.714(1); 7.698(2.12); 7.68(1.63); 7.676(1.93); 7.644(0.4); 7.624(2.95); 7.62(2.68); 7.609(9.56); 7.593(3.22); 7.582(2.76); 7.561(3.64); 7.545(2.5); 7.542(1.99); 7.532(1.01); 7.525(0.86); 7.372(0.61); 7.363(1.02); 7.358(0.94); 7.35(4); 7.339(3.45); 7.332(7.7); 7.321(1.61); 7.317(1.13); 7.305(0.36); 7.301(0.33); 7.146(4.44); 7.141(3.73); 7.131(2.27); 7.127(4.13); 7.123(3.35); 3.018(16); 2.67(0.52); 2.666(0.35); 2.519(0.66); 2.51(41.76); 2.506(91.52); 2.501 (124.86); 2.496(93.07); 2.492(47.95); 2.332(0.75); 2.328(0.96); 2.323 (0.77); 2.074(4.6); 0(12.51); −0.008(0.63) |
| 533 | 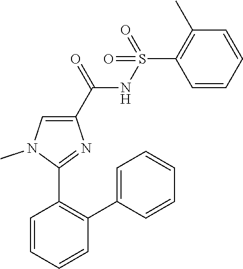 [SMILES: Cc1ccccc1S(=O)(=O)NC(=O)c1cn(C)c(-c2ccccc2-c2ccccc2)n1] | Example 533: ¹H-NMR(400.0 MHz, d₆-DMSO): δ = 8.01(2); 7.993(2.02); 7.99(2.1); 7.827(4.11); 7.689(0.7); 7.684(0.81); 7.669 (1.53); 7.667(1.61); 7.665(1.62); 7.652(1.43); 7.648(1.69); 7.593(3.21); 7.588(1.5); 7.573(4.85); 7.569(3.57); 7.559(2.49); 7.556(2.76); 7.542(1.96); 7.539(2.17); 7.523(0.76); 7.52(0.7); 7.439(1.18); 7.419(1.88); 7.395(2.25); 7.376(1.69); 7.352(0.45); 7.347(0.36); 7.342(0.83); 7.338(0.76); 7.329 (3.14); 7.314(5.52); 7.311(6.13); 7.303(1.48); 7.298(0.85); 7.146(3.57); 7.14(2.83); 7.135(1.73); 7.131(1.74); 7.126(3.3); 7.122(2.71); 2.955(16); 2.674(0.49); 2.67(0.68); 2.666(0.51); 2.589(15.49); 2.523(1.69); 2.518 (2.58); 2.51(37.98); 2.506(79.62); 2.501(106.25); 2.496(76.73); 2.492 (37.08); 2.332(0.43); 2.328(0.61); 2.323(0.45); 2.074(3.37); 0.008(0.36); 0(10.93); −0.008(0.39) |
| 534 | 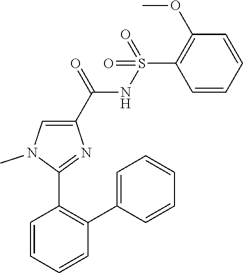 [SMILES: COc1ccccc1S(=O)(=O)NC(=O)c1cn(C)c(-c2ccccc2-c2ccccc2)n1] | Example 534: ¹H-NMR(400.0 MHz, d₆-DMSO): δ = 8.134(0.6); 7.902(1.25); 7.898(1.41); 7.882(1.61); 7.877(5.08); 7.696(0.6); 7.691(0.68); 7.688(0.59); 7.682(0.6); 7.673(1.57); 7.67(1.38); 7.663(0.97); 7.656(0.92); 7.652(1.48); 7.647(1.08); 7.598(1.79); 7.579(1.19); 7.56(3.04); 7.556(2.78); 7.544(1.12); 7.54(1.09); 7.342(0.5); 7.337(0.44); 7.328(1.9); 7.318(1.58); 7.31(3.72); 7.3(0.8); 7.296(0.56); 7.258(1.6); 7.237(1.42); 7.17 (0.84); 7.169(0.91); 7.15(3.65); 7.144(1.94); 7.13(2.76); 7.126(1.76); 3.842 (12.77); 2.933(10.88); 2.51(8.16); 2.506(17.79); 2.501(24.3); 2.496(18.01); 2.492(9.15); 2.074(16); 0(3.08) |
| 535 | 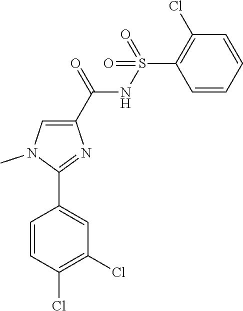 [SMILES: Cn1cc(C(=O)NS(=O)(=O)c2ccccc2Cl)nc1-c1ccc(Cl)c(Cl)c1] | Example 535: ¹H-NMR(400.0 MHz, d₆-DMSO): δ = 8.156(1.59); 8.152(1.74); 8.136(1.8); 8.132(1.92); 8.118(4.27); 8.077(3.13); 8.073(3.13); 7.811(0.77); 7.79(5.19); 7.786(3.92); 7.781(3.39); 7.765(0.56); 7.76(0.63); 7.71(0.41); 7.706(0.45); 7.689(1.2); 7.686(1.17); 7.672(1.49); 7.668(1.54); 7.656(2.02); 7.652(2.73); 7.636(1.08); 7.632(0.84); 7.62(1.19); 7.617(1.03); 7.601(1.5); 7.583(0.76); 7.579(0.71); 3.82(16); 3.796(0.41); 2.67(0.43); 2.519(0.63); 2.51(29.71); 2.506(65.81); 2.501(90.42); 2.497 (66.72); 2.492(33.58); 2.332(0.49); 2.328(0.65); 2.324(0.5); 2.074(1.84); 1.509(0.87); 0(11.1); −0.008(0.51) |
| 536 | 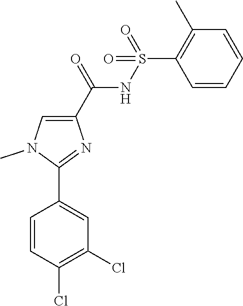 [SMILES: Cc1ccccc1S(=O)(=O)NC(=O)c1cn(C)c(-c2ccc(Cl)c(Cl)c2)n1] | Example 536: ¹H-NMR(400.0 MHz, d₆-DMSO): δ = 8.079(3.4); 8.078(3.13); 8.076(2.75); 8.062(5.45); 8.026(1.56); 8.023(1.68); 8.006(1.75); 8.003(1.75); 7.807(0.35); 7.787(8.19); 7.783(4.38); 7.762 (0.33); 7.587(0.66); 7.584(0.7); 7.568 (1.61); 7.565(1.65); 7.55(1.09); 7.546 (1.06); 7.454(1.01); 7.435(1.58); 7.416(0.74); 7.396(1.78); 7.378(1.47); 3.813(16); 3.796(0.59); 2.675(0.32); 2.671(0.45); 2.666(0.34); 2.611 (12.64); 2.524(0.96); 2.519(1.48); 2.51(27.05); 2.506(57.02); 2.502(75.92); 2.497(54.43); 2.492(26.23); 2.333(0.34); 2.328(0.47); 2.324(0.34); 2.074 (0.64); 1.509(1.21); 0(8.2) |
| 537 | 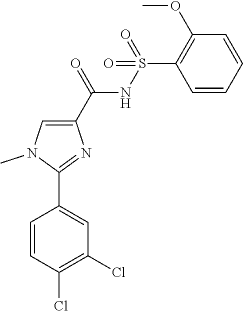 [SMILES: COc1ccccc1S(=O)(=O)NC(=O)c1cn(C)c(-c2ccc(Cl)c(Cl)c2)n1] | Example 537: ¹H-NMR(400.0 MHz, d₆-DMSO): δ = 8.097(4.88); 8.081(3.66); 8.078(2.59); 7.904(1.53); 7.9(1.74); 7.884(1.71); 7.88(1.8); 7.79(7.07); 7.787(7.08); 7.685(0.69); 7.681(0.76); 7.663(1.34); 7.66(1.18); 7.646(0.88); 7.641(0.88); 7.234(2.01); 7.213(1.81); 7.163(1.11); 7.144(1.95); 7.124(0.98); 3.85(16); 3.824(14.31); 3.796(0.57); 2.67(0.32); 2.524(0.7); 2.51(19.26); 2.506(41.28); 2.501(56.17); 2.497(41.91); 2.492 (21.33); 2.328(0.34); 2.074(4.73); 1.508(1.4); 0(5.46) |
| 538 | 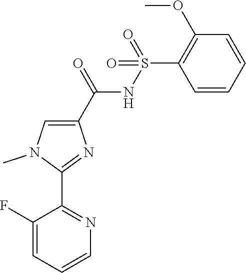 [SMILES: COc1ccccc1S(=O)(=O)NC(=O)c1cn(C)c(-c2ncccc2F)n1] | Example 538: ¹H-NMR(400.0 MHz, d₆-DMSO): δ = 8.58(1.04); 8.576(1.78); 8.573(1.18); 8.568(1.18); 8.565(1.82); 8.198(1.45); 7.975(0.87); 7.972(0.9); 7.954(1.09); 7.95(1.43); 7.946(1.02); 7.928(1.01); 7.925(1.02); 7.908(1.45); 7.904(1.61); 7.888(1.59); 7.884(1.63); 7.684 (0.53); 7.665(1.08); 7.657(1.16); 7.646(2); 7.635(1.64); 7.625(1.17); 7.614 (0.72); 7.237(1.58); 7.216(1.45); 7.167(0.88); 7.148(1.6); 7.128(0.79); 3.898 (0.35); 3.889(0.33); 3.849(15.39); 3.838(16); 3.318(4.61); 2.679(0.37); 2.674(0.83); 2.67(1.15); 2.665(0.85); 2.523(2.65); 2.518(4.09); 2.51(68.59); 2.505(145.84); 2.501(196.61); 2.496(143.11); 2.492(70.26); 2.336(0.4); 2.332(0.83); 2.328(1.2); 2.323(0.88); 2.074(1.55); 0.146(0.9); 0.008(6.7); 0(218.96); −0.009(9.25); −0.15(0.94) |
| 539 | 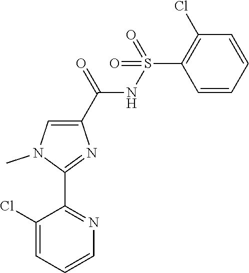 [SMILES: Cn1cc(C(=O)NS(=O)(=O)c2ccccc2Cl)nc1-c1ncccc1Cl] | Example 539: ¹H-NMR(400.0 MHz, d₆-DMSO): δ = 8.693(1.96); 8.689(2.05); 8.681(2.09); 8.678(2.05); 8.199(4.39); 8.177 (1.99); 8.174(2.09); 8.167(1.75); 8.164(1.87); 8.157(2.29); 8.153(2.23); 8.148(1.94); 8.144(1.92); 7.727(0.4); 7.723(0.43); 7.707(1.31); 7.69(1.59); 7.686(1.61); 7.676(2.17); 7.672(2.86); 7.656(1.06); 7.634(1.23); 7.628 (2.34); 7.616(3.24); 7.607(2.12); 7.596(2.35); 3.666(16); 2.67(0.44); 2.524 (0.77); 2.506(60.87); 2.502(80.82); 2.497(59.05); 2.332(0.4); 2.328(0.54); 2.324(0.4); 2.074(1.04); 0.008(0.38); 0(12.75) |
| 540 | 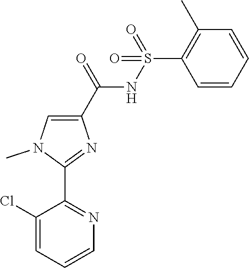 [SMILES: Cc1ccccc1S(=O)(=O)NC(=O)c1cn(C)c(-c2ncccc2Cl)n1] | Example 540: ¹H-NMR(400.0 MHz, d₆-DMSO): δ = 8.686(2.24); 8.677(2.18); 8.674(2.25); 8.174(2.03); 8.171(2.16); 8.153 (2.22); 8.15(2.29); 8.126(4.44); 8.032(1.97); 8.012(2.08); 7.624(1.83); 7.612 (1.81); 7.603(1.78); 7.592(2.19); 7.569(1.88); 7.551(1.23); 7.456(1.18); 7.437(1.93); 7.418(0.9); 7.402(2.12); 7.382(1.72); 3.648(16); 3.467(0.51); 3.338(0.72); 3.312(0.68); 3.196(0.37); 2.671(0.47); 2.61(13.84); 2.502 (74.35); 2.328(0.44); 2.074(0.76); 0(66.91); −0.15(0.33) |
| 541 | 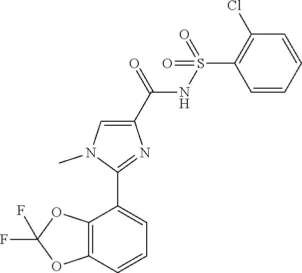 [SMILES: Cn1cc(C(=O)NS(=O)(=O)c2ccccc2Cl)nc1-c1cccc2c1OC(F)(F)O2] | Example 541: ¹H-NMR(400.0 MHz, d₆-DMSO): δ = 8.201(1.3); 8.159(1.41); 8.156(1.51); 8.14(1.52); 8.136(1.59); 7.694(0.93); 7.675(1.12); 7.66(1.9); 7.643(0.77); 7.624(0.85); 7.604(1.26); 7.594(1.93); 7.592(2.17); 7.574(2.2); 7.572(2.33); 7.488(1.56); 7.486(1.73); 7.468(2.43); 7.465(2.29); 7.386(2.2); 7.366(3.32); 7.346(1.33); 3.72(16); 3.7(0.33); 2.675 (0.76); 2.67(1.1); 2.666(0.79); 2.524(2.47); 2.519(3.88); 2.51(67.04); 2.506 (141.06); 2.501(188.7); 2.497(135.28); 2.492(64.99); 2.333(0.83); 2.328 (1.15); 2.324(0.85); 1.513(0.45); 1.506(0.66); 0.008(2.36); 0(74.97); −0.008 (2.82); −0.15(0.33) |
| 542 | 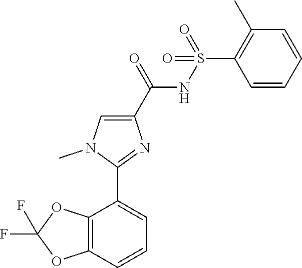 [SMILES: Cc1ccccc1S(=O)(=O)NC(=O)c1cn(C)c(-c2cccc3c2OC(F)(F)O3)n1] | Example 542: ¹H-NMR(400.0 MHz, d₆-DMSO): δ = 8.15(4.57); 8.03(1.85); 8.011(2.01); 7.592(2.57); 7.572(4.09); 7.553(1.19); 7.487(1.83); 7.467(2.86); 7.458(1.24); 7.438(1.8); 7.419(0.85); 7.402(2.01); 7.387(3.14); 7.367(3.32); 7.347(1.33); 3.708(16); 2.671(0.47); 2.667(0.35); 2.613(13.86); 2.506(59.22); 2.502(75.71); 2.497(53.62); 2.329(0.45); 2.075 (0.41); 1.514(0.34); 0.008(0.54); 0(15.16); −0.008(0.56) |
| 543 | 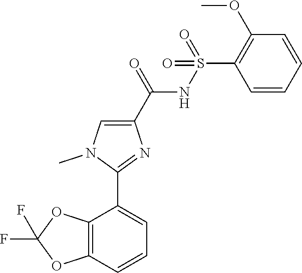 [SMILES: COc1ccccc1S(=O)(=O)NC(=O)c1cn(C)c(-c2cccc3c2OC(F)(F)O3)n1] | Example 543: ¹H-NMR(400.0 MHz, d₆-DMSO): δ = 8.189(4.71); 7.91(1.55); 7.905(1.69); 7.89(1.73); 7.886(1.75); 7.692(0.71); 7.688(0.73); 7.67(1.26); 7.652(0.89); 7.648(0.84); 7.595(1.7); 7.592(1.89); 7.575(2.12); 7.572(2.19); 7.501(1.52); 7.498(1.64); 7.48(2.35); 7.478(2.11); 7.391(2.09); 7.37(3.21); 7.35(1.32); 7.246(1.94); 7.225(1.76); 7.17(1.05); 7.168(1.03); 7.15(1.91); 7.132(0.97); 3.866(16); 3.726(14.4); 2.524(0.42); 2.52(0.65); 2.511(11.37); 2.507(24.01); 2.502(32.06); 2.498(22.74); 2.493 (10.79); 0(8.24) |
| 544 | 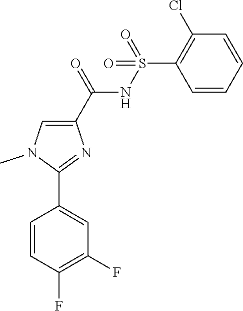 [SMILES: Cn1cc(C(=O)NS(=O)(=O)c2ccccc2Cl)nc1-c1ccc(F)c(F)c1] | Example 544: ¹H-NMR(400.0 MHz, d₆-DMSO): δ = 8.155(1.7); 8.151(1.79); 8.135(1.95); 8.131(1.92); 8.108(5.11); 7.929(0.79); 7.913(0.81); 7.908(0.91); 7.903(0.94); 7.898(0.84); 7.884(0.77); 7.878 (0.77); 7.705(0.45); 7.701(0.48); 7.685(1.38); 7.668(1.78); 7.664(1.99); 7.652(2.83); 7.648(3.44); 7.642(2.12); 7.637(2.9); 7.629(2.25); 7.625(1.74); 7.617(2.13); 7.613(2.31); 7.594(2.51); 7.58(0.91); 7.576(0.85); 3.801(16); 2.506(36.75); 2.502(47.85); 2.497(35.05); 0.008(2.57); 0(53.34); −0.008(3) |
| 545 | 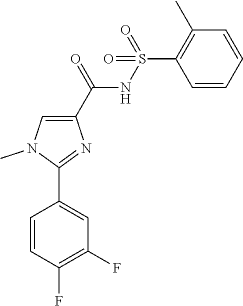 [SMILES: Cc1ccccc1S(=O)(=O)NC(=O)c1cn(C)c(-c2ccc(F)c(F)c2)n1] | Example 545: ¹H-NMR(400.0 MHz, d₆-DMSO): δ = 8.049(5.59); 8.024(1.88); 8.004(2); 7.93(0.76); 7.927(0.78); 7.911(0.82); 7.907(0.93); 7.902(0.96); 7.897(0.87); 7.882(0.82); 7.877(0.81); 7.663 (0.38); 7.659(0.39); 7.641(1.45); 7.637(1.69); 7.631(2.1); 7.613(1.24); 7.607(1.23); 7.586(1.84); 7.566(2.23); 7.548(1.24); 7.545(1.18); 7.452 (1.15); 7.433(1.83); 7.415(0.86); 7.395(2.03); 7.376(1.69); 3.793(16); 2.671 (0.38); 2.61(13.76); 2.506(47.34); 2.501(60.74); 2.497(44.85); 2.328(0.36); 2.074(0.34); 0.146(0.35); 0(73.07); −0.15(0.34) |
| 546 | 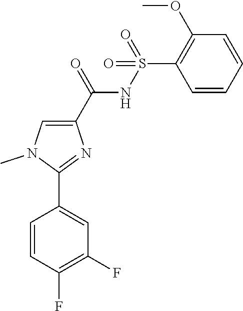 [SMILES: COc1ccccc1S(=O)(=O)NC(=O)c1cn(C)c(-c2ccc(F)c(F)c2)n1] | Example 546: ¹H-NMR(400.0 MHz, d₆-DMSO): δ = 8.084(4.38); 7.934(0.66); 7.929(0.71); 7.914(0.78); 7.904(2.33); 7.899 (2.48); 7.884(2.36); 7.88(2.47); 7.685(0.71); 7.68(0.75); 7.663(1.81); 7.645 (1.97); 7.641(2.12); 7.632(1.97); 7.612(1.12); 7.606(1.09); 7.586(1.15); 7.564(0.48); 7.234(2.1); 7.214(1.92); 7.163(1.14); 7.144(2.06); 7.125(1.05); 3.851(16); 3.804(14.69); 2.506(35.3); 2.502(46.18); 2.497(33.92); 0.008 (3.01); 0(60.92); −0.008(3.36) |
| 547 | 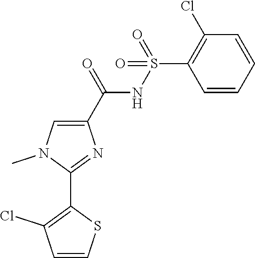 [SMILES: Cn1cc(C(=O)NS(=O)(=O)c2ccccc2Cl)nc1-c1sccc1Cl] | Example 547: ¹H-NMR(400.0 MHz, d₆-DMSO): δ = 8.211(4.79); 8.158(1.78); 8.155(1.85); 8.138(1.98); 8.136(2); 7.963(3.48); 7.95(3.62); 7.718(0.49); 7.701(1.51); 7.684(1.74); 7.681(1.77); 7.665(3.05); 7.649(1.21); 7.627(1.27); 7.607(1.81); 7.59(0.83); 7.586(0.76); 7.277(3.7); 7.264(3.65); 3.638(16); 3.616(0.51); 2.506(38.16); 2.502(48.14); 2.328 (0.33); 1.507(0.71); 0(52.75) |
| 548 | 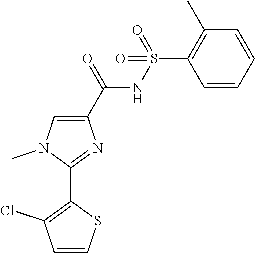 [SMILES: Cc1ccccc1S(=O)(=O)NC(=O)c1cn(C)c(-c2sccc2Cl)n1] | Example 548: ¹H-NMR(400.0 MHz, d₆-DMSO): δ = 8.134(2.42); 8.022(1.73); 8.001(1.85); 7.957(3.33); 7.944(3.49); 7.579 (0.66); 7.562(1.47); 7.543(1); 7.447(0.98); 7.428(1.61); 7.409(0.8); 7.393 (1.78); 7.374(1.46); 7.274(3.73); 7.261(3.62); 3.622(16); 3.442(0.45); 3.328 (1.23); 3.183(0.34); 2.67(0.79); 2.606(13.63); 2.506(108.23); 2.501(137.96); 2.497(101.07); 2.328(0.82); 2.074(0.64); 1.506(0.87); 0.146(0.71); 0 (155.12); −0.149(0.73) |
| 549 | 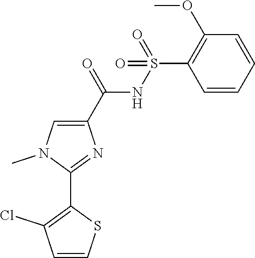 [SMILES: COc1ccccc1S(=O)(=O)NC(=O)c1cn(C)c(-c2sccc2Cl)n1] | Example 549: ¹H-NMR(400.0 MHz, d₆-DMSO): δ = 8.174 (4.3); 7.960 (3.4); 7.947 (3.6); 7.897 (1.7); 7.893 (1.7); 7.878 (1.8); 7.874 (1.8); 7.679 (0.8); 7.675 (0.8); 7.658 (1.5); 7.640 (0.9); 7.636 (0.9); 7.278 (3.7); 7.265 (3.5); 7.233 (2.2); 7.212 (2.1); 7.158 (1.2); 7.138 (2.1); 7.120 (1.1); 5.756 (4.9); 3.898 (0.8); 3.853 (16.0); 3.639 (15.0); 3.318 (2.4); 3.166 (0.8); 2.670 (0.7); 2.505 (95.8); 2.501 (121.1); 2.497 (89.2); 2.328 (0.7); 0.146 (0.6); 0.000 (139.1); −0.150 (0.7) |
| 550 | 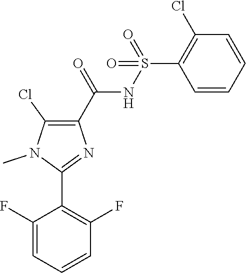 [SMILES: Cn1c(-c2c(F)cccc2F)nc(C(=O)NS(=O)(=O)c2ccccc2Cl)c1Cl] | Example 550: ¹H-NMR(400.0 MHz, d₆-DMSO): δ = 20.006(0.38); 12.49(0.32); 12.336(0.34); 12.268(0.32); 8.155(6.91); 8.135 (7.69); 7.789(1.27); 7.768(2.99); 7.75(5.53); 7.73(4.94); 7.709(5.86); 7.688 (6.05); 7.672(10.38); 7.656(4.21); 7.635(4.44); 7.615(6.45); 7.598(2.98); 7.393(9.55); 7.372(16); 7.352(8.52); 3.661(0.44); 3.485(51.1); 3.33(6.81); 3.151(0.45); 2.67(4.43); 2.505(627.31); 2.501(798.3); 2.497(607.3); 2.369 (0.5); 2.328(4.66); 2.074(8.35); 0.146(3.78); 0(780.53); −0.15(3.91) |
| 551 | 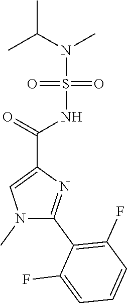 [SMILES: CC(C)N(C)S(=O)(=O)NC(=O)c1cn(C)c(-c2c(F)cccc2F)n1] | Example 551: ¹H-NMR(300.1 MHz, d₆-DMSO): δ = 8.179(3.72); 8.141(0.37); 7.755(0.33); 7.732(0.74); 7.726(0.66); 7.704 (1.38); 7.682(0.67); 7.676(0.87); 7.653(0.37); 7.364(2.48); 7.338(3.54); 7.31 (1.93); 7.3(0.36); 6.539(0.35); 4.148(0.32); 4.125(0.87); 4.103(1.28); 4.08 (0.94); 4.058(0.36); 3.575(11.38); 3.329(19.33); 2.793(16); 2.514(19.14); 2.508(37.71); 2.502(49.91); 2.496(34.63); 1.084(13.06); 1.062(12.88); 0(3.63) |
| 552 | 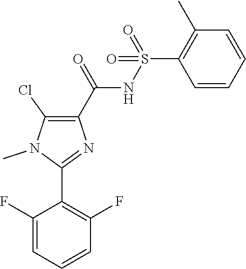 [SMILES: Cc1ccccc1S(=O)(=O)NC(=O)c1nc(-c2c(F)cccc2F)n(C)c1Cl] | Example 552: ¹H-NMR(400.0 MHz, d₆-DMSO): δ = 8.036(2.09); 8.017(2.22); 7.786(0.37); 7.769(0.85); 7.765(0.88); 7.748 (1.61); 7.731(0.9); 7.727(1.02); 7.71(0.42); 7.595(0.79); 7.575(1.87); 7.557 (1.23); 7.463(1.2); 7.444(1.93); 7.425(0.91); 7.402(2.23); 7.39(3.41); 7.369 (4.73); 7.349(2.48); 3.479(14.99); 3.322(3.94); 2.671(0.82); 2.602(16); 2.506(109.39); 2.502(145.07); 2.497(108.35); 2.328(0.85); 2.074(1.5); 0.146(0.67); 0.008(6.03); 0(145.2); −0.15(0.71) |
| 553 | 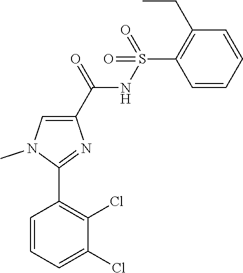 [SMILES: CCc1ccccc1S(=O)(=O)NC(=O)c1cn(C)c(-c2cccc(Cl)c2Cl)n1] | Example 553: ¹H-NMR(400.0 MHz, d₆-DMSO): δ = 8.116(4.88); 8.085(0.43); 8.023(2); 8.003(2.12); 7.871(1.68); 7.865(1.78); 7.853(1.84); 7.847(1.95); 7.631(0.79); 7.611(1.8); 7.594(1.16); 7.569(0.41); 7.562(0.94); 7.549(3.51); 7.544(5.33); 7.526(2.77); 7.507(0.82); 7.45(2.54); 7.443(1.54); 7.431(2.18); 7.424(2.08); 7.404(0.91); 4.044(1.41); 3.507(16); 3.076(1.15); 3.057(3.66); 3.038(3.74); 3.02(1.23); 2.67(0.41); 2.506(55.6); 2.501(72.25); 2.497(52.18); 2.328(0.42); 2.074(0.36); 1.183(4.27); 1.165 (9.19); 1.146(4.17); 0.146(0.35); 0.008(3.24); 0(78.9); −0.008(3.36); −0.15 (0.37) |
| 554 | 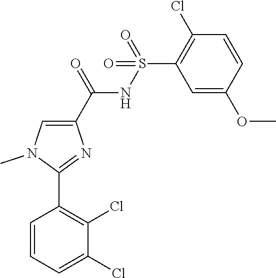 | Example 554: ¹H-NMR(400.0 MHz, d₆-DMSO): δ = 8.187(3.69); 7.88(1.51); 7.876(1.67); 7.86(1.73); 7.856(1.81); 7.604(3.06); 7.596(3.12); 7.59(1.11); 7.585(1.18); 7.57(2.7); 7.564(3.61); 7.554(2.63); 7.541(2.93); 7.534(2.86); 7.515(0.91); 7.278(1.43); 7.27(1.43); 7.256(1.21); 7.248(1.19); 4.049(0.76); 3.85(16); 3.526(13.76); 2.671(0.36); 2.506 (50.16); 2.502(63.38); 2.328(0.39); 2.074(1.24); 0(56.31) |
| 555 | 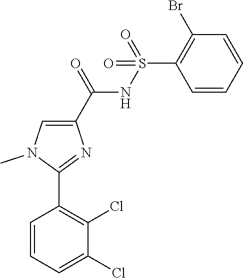 [SMILES: Cn1cc(C(=O)NS(=O)(=O)c2ccccc2Br)nc1-c1cccc(Cl)c1Cl] | Example 555: ¹H-NMR(400.0 MHz, d₆-DMSO): δ = 8.201(5.21); 8.186(1.95); 8.182(2.07); 8.166(2.03); 8.162(2.11); 8.153(1.3); 7.878(1.71); 7.874(1.85); 7.859(2.06); 7.854(2.51); 7.845(2.17); 7.826 (2.46); 7.662(1.37); 7.644(2.29); 7.627(1.45); 7.599(1.81); 7.583(2.83); 7.567(3.26); 7.563(3.17); 7.552(2.7); 7.533(2.87); 7.514(1.02); 4.05(4.37); 3.526(16); 3.488(0.34); 2.671(0.56); 2.506(79.27); 2.502(101.01); 2.328 (0.6); 2.074(1.83); 1.513(0.4); 1.508(0.6); 0.146(0.39); 0(80.78); −0.149 (0.42) |
| 556 | 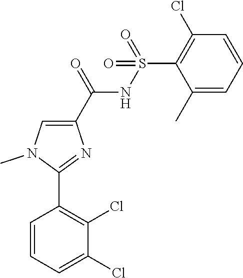 [SMILES: Cc1cccc(Cl)c1S(=O)(=O)NC(=O)c1cn(C)c(-c2cccc(Cl)c2Cl)n1] | Example 556: ¹H-NMR(400.0 MHz, d₆-DMSO): δ = 8.173(1.79); 8.134(0.4); 7.871(1.69); 7.866(1.77); 7.852(1.85); 7.847(1.91); 7.574(0.62); 7.568(1.01); 7.554(3.01); 7.549(3.07); 7.546(3.5); 7.527(3.16); 7.507(2.01); 7.489(4.26); 7.409(1.35); 7.404(1.3); 7.395(1.07); 4.039(2.05); 3.515(16); 3.409(0.51); 3.389(0.49); 3.337(0.54); 3.293(0.45); 3.238(0.38); 2.74(15.23); 2.675(0.97); 2.67(1.28); 2.666(1); 2.506(174.31); 2.501 (227.98); 2.497(165.7); 2.333(1); 2.328(1.33); 2.324(1.03); 2.074(0.94); 0.146(0.45); 0.008(4.51); 0(100.32); −0.008(4.79); −0.15(0.5) |
| 557 | 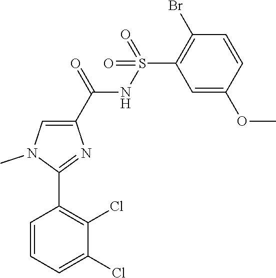 | Example 557: ¹H-NMR(400.0 MHz, d₆-DMSO): δ = 8.159(2.75); 7.932(3.44); 7.926(3.93); 7.872(1.64); 7.866(1.84); 7.854 (3.02); 7.848(3.1); 7.833(1.5); 7.827(1.37); 7.571(0.35); 7.565(0.88); 7.551 (3.6); 7.547(4.67); 7.529(2.62); 7.51(0.76); 7.231(2.49); 7.209(2.32); 4.054 (0.38); 3.852(16); 3.515(15.32); 2.755(0.78); 2.506(40.59); 2.502(53.87); 2.497(40.81); 2.328(0.33); 2.074(1.12); 0(26.19) |
| 558 | 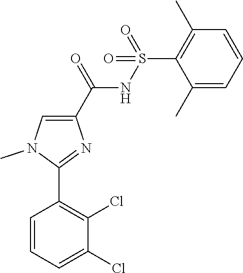 [SMILES: Cc1cccc(C)c1S(=O)(=O)NC(=O)c1cn(C)c(-c2cccc(Cl)c2Cl)n1] | Example 558: ¹H-NMR(400.0 MHz, d₆-DMSO): δ = 8.117(2.15); 7.868(0.92); 7.863(0.94); 7.85(1.04); 7.844(1.03); 7.564(0.58); 7.55(1.83); 7.544(2.46); 7.524(1.43); 7.506(0.44); 7.409(0.61); 7.39(1.31); 7.371(0.92); 7.233(2.94); 7.214(2.36); 4.04(1.19); 3.504(8.33); 2.675(16); 2.501(61.18); 2.328(0.38); 2.074(1.37); 0(13.88) |
| 559 | 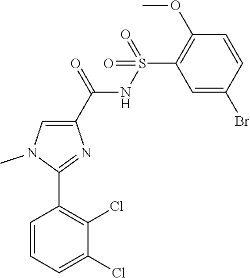 [SMILES: COc1ccc(Br)cc1S(=O)(=O)NC(=O)c1cn(C)c(-c2cccc(Cl)c2Cl)n1] | Example 559: ¹H-NMR(400.0 MHz, d₆-DMSO): δ = 8.176(3.71); 7.934(3.25); 7.928(3.75); 7.874(1.72); 7.867(2.38); 7.856 (2.49); 7.85(2.15); 7.842(1.75); 7.836(1.53); 7.573(0.39); 7.567(0.89); 7.554 (3.49); 7.549(5.1); 7.531(2.61); 7.512(0.78); 7.238(2.69); 7.216(2.52); 4.06 (0.45); 3.856(16); 3.518(15.2); 3.337(0.34); 2.671(0.49); 2.506(66.2); 2.502 (84.53); 2.498(61.82); 2.328(0.51); 2.074(1.12); 0.008(2.2); 0(44.15) |
| 560 | 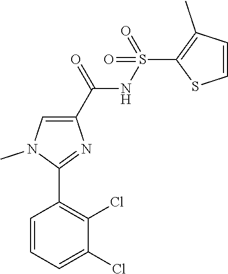 [SMILES: Cc1ccsc1S(=O)(=O)NC(=O)c1cn(C)c(-c2cccc(Cl)c2Cl)n1] | Example 560: ¹H-NMR(400.0 MHz, d₆-DMSO): δ = 8.143(4.1); 7.873(3.72); 7.86(2.65); 7.855(2.44); 7.85(2.1); 7.575(0.58); 7.57(1.01); 7.556(3.16); 7.55(3.85); 7.529(2.78); 7.51(0.87); 7.031(2.78); 7.018(2.71); 3.515(15.69); 3.487(0.48); 3.408(0.33); 3.362(0.32); 3.335 (0.37); 2.67(0.6); 2.506(85.72); 2.501(108.62); 2.497(80.37); 2.458(16); 2.328(0.66); 2.074(1.77); 0(52.14) |
| 561 | 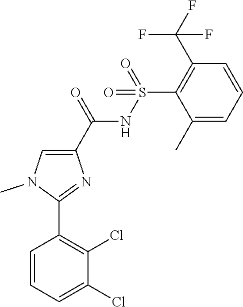 [SMILES: Cc1cccc(C(F)(F)F)c1S(=O)(=O)NC(=O)c1cn(C)c(-c2cccc(Cl)c2Cl)n1] | Example 561: ¹H-NMR(400.0 MHz, d₆-DMSO): δ = 8.127(4.83); 7.889(1.96); 7.885(2.04); 7.869(2.46); 7.865(2.45); 7.86(1.31); 7.848(1.96); 7.836(1.41); 7.702(5.07); 7.691(4.06); 7.606(1.15); 7.602 (1.35); 7.587(2.85); 7.583(2.41); 7.562(2.6); 7.542(3.18); 7.523(1.21); 4.05 (0.69); 3.526(16); 2.74(15.18); 2.67(0.4); 2.506(59.27); 2.502(75.06); 2.497 (54.46); 2.329(0.49); 2.074(5.36); 0.008(1.85); 0(41.07); −0.008(2.21) |
| 562 | 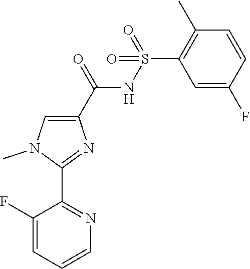 [SMILES: Cc1ccc(F)cc1S(=O)(=O)NC(=O)c1cn(C)c(-c2ncccc2F)n1] | Example 562: ¹H-NMR(400.0 MHz, d₆-DMSO): δ = 8.581(1.93); 8.572(1.4); 8.569(1.95); 8.181(5.6); 7.976(0.96); 7.973(0.96); 7.954(1.25); 7.951(1.67); 7.929(1.11); 7.926(1.06); 7.781(1.33); 7.775 (1.43); 7.758(1.27); 7.754(1.39); 7.666(1); 7.655(1.63); 7.644(1.75); 7.634 (1.32); 7.623(0.79); 7.477(1.57); 7.47(2.1); 7.467(2.28); 7.457(2.07); 7.452 (3.42); 3.815(16); 2.578(11.69); 2.507(27.91); 2.503(36.69); 2.498(26.95); 0.008(1.05); 0(25.04) |
| 563 | 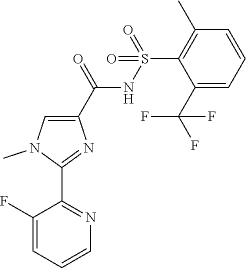 [SMILES: Cc1cccc(C(F)(F)F)c1S(=O)(=O)NC(=O)c1cn(C)c(-c2ncccc2F)n1] | Example 563: ¹H-NMR(400.0 MHz, d₆-DMSO): δ = 8.585(1.94); 8.574(2); 8.179(5.4); 7.978(1.02); 7.955(1.76); 7.931(1.19); 7.884(1.05); 7.874(1.65); 7.86(1.44); 7.746(0.32); 7.723(3.47); 7.712(5.14); 7.672(0.94); 7.661(1.67); 7.65(1.65); 7.64(1.4); 7.629(0.76); 3.821(16); 3.025(0.5); 2.751(14.61); 2.712(0.34); 2.507(28.86); 2.503(39.59); 2.499 (31.23); 0.008(0.76); 0(29.29) |
| 564 | 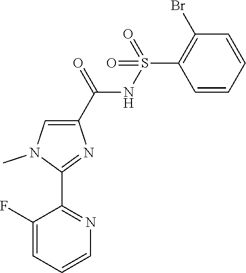 [SMILES: Cn1cc(C(=O)NS(=O)(=O)c2ccccc2Br)nc1-c1ncccc1F] | Example 564: ¹H-NMR(400.0 MHz, d₆-DMSO): δ = 8.579(1.97); 8.567(2); 8.248(4.39); 8.2(1.72); 8.196(1.77); 8.181(1.9); 8.177 (1.86); 7.971(1); 7.968(0.99); 7.95(1.29); 7.946(1.7); 7.924(1.14); 7.921 (1.1); 7.855(1.8); 7.852(1.8); 7.835(2.19); 7.833(2.09); 7.68(0.78); 7.677 (0.82); 7.661(2.78); 7.651(1.87); 7.64(2.92); 7.63(1.49); 7.618(1.8); 7.613 (1.37); 7.598(1.61); 7.594(1.6); 7.579(0.67); 7.575(0.59); 3.832(16); 2.507 (31.07); 2.502(40.84); 2.498(30.34); 2.075(0.47); 0.008(1.67); 0(40.83) |
| 565 | 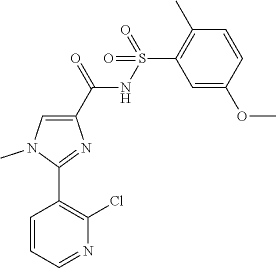 | Example 565: ¹H-NMR(400.0 MHz, d₆-DMSO): δ = 8.619(1.58); 8.614(1.72); 8.607(1.75); 8.602(1.68); 8.14(4.43); 8.095(1.55); 8.09(1.61); 8.076(1.84); 8.071(2.04); 7.634(1.55); 7.622(1.56); 7.615(1.54); 7.603(1.44); 7.532(2.57); 7.525(2.7); 7.501(0.58); 7.494(0.61); 7.314(1.66); 7.293(2.23); 7.278(0.51); 7.169(1.44); 7.162(1.47); 7.148(1.29); 7.141 (1.17); 3.804(16); 3.794(4.14); 3.629(3.1); 3.549(13.21); 2.517(14.03); 2.506(30.08); 2.502(38.39); 2.498(29.33); 2.074(0.54); 0.008(1.89); 0(35.9) |
| 566 | 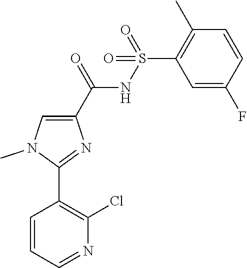 [SMILES: Cc1ccc(F)cc1S(=O)(=O)NC(=O)c1cn(C)c(-c2cccnc2Cl)n1] | Example 566: ¹H-NMR(400.0 MHz, d₆-DMSO): δ = 8.621(1.72); 8.616(1.85); 8.609(1.89); 8.604(1.76); 8.314(0.41); 8.127(1.2); 8.097(1.69); 8.078(1.75); 7.755(1.32); 7.733(1.43); 7.636(1.65); 7.624 (1.77); 7.616(1.69); 7.604(1.54); 7.445(2.15); 7.429(2.45); 3.774(0.38); 3.752(0.4); 3.621(1.14); 3.55(16); 3.388(3.38); 3.011(0.35); 2.997(0.35); 2.67(1.57); 2.566(12.44); 2.501(289.51); 2.328(1.75); 2.074(1.51); 1.239 (0.35); 0.146(1.03); 0(209.15); −0.15(1.03) |
| 567 | 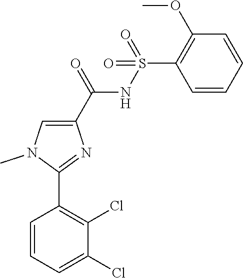 [SMILES: COc1ccccc1S(=O)(=O)NC(=O)c1cn(C)c(-c2cccc(Cl)c2Cl)n1] | Example 567: ¹H-NMR(400.0 MHz, d₆-DMSO): δ = 8.167(4.39); 7.903(1.67); 7.899(1.8); 7.883(1.9); 7.88(1.98); 7.873(1.74); 7.867(1.71); 7.855(1.72); 7.849(1.82); 7.683(0.78); 7.665(1.63); 7.647 (0.97); 7.572(0.33); 7.566(0.85); 7.553(3.67); 7.549(4.83); 7.531(2.47); 7.512(0.74); 7.241(2.3); 7.22(2.11); 7.164(1.26); 7.145(2.29); 7.126(1.16); 3.849(16); 3.514(14.96); 3.488(0.35); 3.336(0.54); 2.502(40.02); 2.074 (3.37); 0(30.68) |
| 568 | 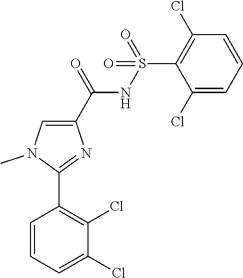 [SMILES: Cn1cc(C(=O)NS(=O)(=O)c2c(Cl)cccc2Cl)nc1-c1cccc(Cl)c1Cl] | Example 568: ¹H-NMR(400.0 MHz, d₆-DMSO): δ = 8.183(5.15); 7.896(1.9); 7.892(1.96); 7.876(2.17); 7.873(2.1); 7.633(3.02); 7.63(3.37); 7.612(7.71); 7.601(3.06); 7.597(2.61); 7.567(4.87); 7.547(4.14); 7.528(2.12); 4.055(0.33); 3.542(16); 2.506(29.41); 2.502(37.13); 2.498 (28.2); 2.074(0.99); 1.508(0.33); 0(29.7) |
| 569 | 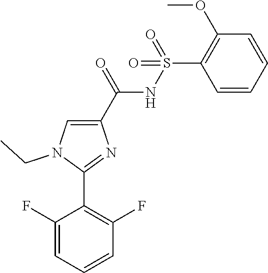 [SMILES: CCn1cc(C(=O)NS(=O)(=O)c2ccccc2OC)nc1-c1c(F)cccc1F] | Example 569: ¹H-NMR(400.0 MHz, d₆-DMSO): δ = 8.322(2.21); 7.906(1.61); 7.902(1.7); 7.887(1.76); 7.883(1.76); 7.735(0.74); 7.714(1.36); 7.692(0.98); 7.682(0.82); 7.663(1.43); 7.646(0.8); 7.361(2.31); 7.341(3.66); 7.321(2); 7.24(1.93); 7.218(1.78); 7.163(1.08); 7.144(1.96); 7.125(1.01); 3.874(1.09); 3.855(4.15); 3.847(16); 3.838(4.23); 3.819(1.22); 3.323(4.7); 2.67(0.48); 2.505(67.98); 2.502(87.69); 2.498(67.66); 2.328 (0.5); 1.515(0.39); 1.269(3.64); 1.251(7.65); 1.232(3.63); 0(39.13) |
| 570 | 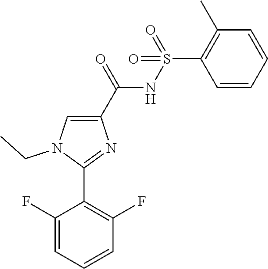 [SMILES: CCn1cc(C(=O)NS(=O)(=O)c2ccccc2C)nc1-c1c(F)cccc1F] | Example 570: ¹H-NMR(400.0 MHz, d₆-DMSO): δ = 8.276(5.64); 8.034(2.24); 8.015(2.37); 7.751(0.38); 7.733(0.92); 7.73(0.9); 7.712(1.66); 7.695(0.99); 7.692(1.04); 7.675(0.43); 7.587(0.91); 7.57(2.15); 7.552(1.42); 7.455(1.36); 7.436(2.2); 7.417(1.04); 7.401(2.43); 7.382(2.01); 7.356(2.85); 7.336(4.5); 7.316(2.48); 3.869(1.29); 3.851(3.97); 3.832(4.06); 3.814(1.38); 3.337(0.33); 3.323(0.32); 2.611(16); 2.506(42.27); 2.502 (54.62); 2.498(41.61); 2.329(0.32); 2.075(0.79); 1.515(0.4); 1.255(4.55); 1.237(9.63); 1.219(4.5); 0(27.54) |
| 571 | 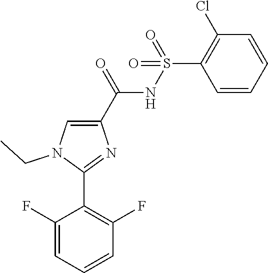 [SMILES: CCn1cc(C(=O)NS(=O)(=O)c2ccccc2Cl)nc1-c1c(F)cccc1F] | Example 571: ¹H-NMR(400.0 MHz, d₆-DMSO): δ = 8.35(9.2); 8.17(3.31); 8.166(3.48); 8.15(3.63); 8.147(3.67); 7.754(0.64); 7.737(1.54); 7.733(1.49); 7.726(1.22); 7.721(1.75); 7.716(2.97); 7.706 (3.14); 7.702(3.15); 7.695(2.14); 7.689(3.58); 7.685(3.46); 7.674(4.78); 7.671(5.67); 7.655(2.19); 7.651(1.55); 7.632(2.37); 7.628(2.05); 7.612 (3.18); 7.594(1.56); 7.59(1.37); 7.36(4.68); 7.34(7.03); 7.32(4.02); 3.883 (1.93); 3.865(6.27); 3.847(6.44); 3.828(2.17); 2.508(34.87); 2.503(47.53); 2.499(35.95); 2.33(0.36); 2.076(0.76); 1.515(0.69); 1.271(7.24); 1.253(16); 1.234(7.54); 0.008(0.87); 0(35.98); −0.008(2.3) |
| 572 | 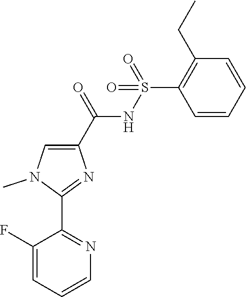 [SMILES: CCc1ccccc1S(=O)(=O)NC(=O)c1cn(C)c(-c2ncccc2F)n1] | Example 572: ¹H-NMR(400.0 MHz, d₆-DMSO): δ = 8.575(1.97); 8.563(2.03); 8.163(5.31); 8.031(1.93); 8.014(1.96); 8.011 (1.98); 7.969(0.98); 7.966(0.95); 7.944(1.71); 7.922(1.13); 7.657(0.95); 7.647(1.67); 7.636(2.49); 7.625(1.67); 7.616(2.28); 7.601(1.25); 7.598 (1.17); 7.456(2.55); 7.433(2.51); 7.412(0.98); 3.812(16); 3.081(1.07); 3.062(3.35); 3.044(3.43); 3.025(1.16); 2.507(22.57); 2.502(29.36); 2.498 (21.97); 1.186(4.02); 1.167(8.6); 1.148(3.95); 0(17.13) |
| 573 | 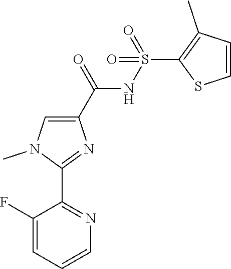 [SMILES: Cc1ccsc1S(=O)(=O)NC(=O)c1cn(C)c(-c2ncccc2F)n1] | Example 573: ¹H-NMR(400.0 MHz, d₆-DMSO): δ = 8.576(1.91); 8.565(1.99); 8.178(3.63); 7.972(0.96); 7.969(0.97); 7.947 (1.68); 7.925(1.15); 7.922(1.1); 7.884(2.03); 7.872(2.06); 7.659(0.95); 7.648(1.57); 7.637(1.66); 7.627(1.28); 7.616(0.75); 7.036(2.67); 7.024 (2.63); 3.819(16); 3.638(0.35); 3.623(0.33); 3.444(0.54); 3.426(0.54); 3.382(0.54); 3.302(0.46); 3.218(0.33); 3.202(0.32); 2.67(0.55); 2.506 (79.81); 2.502(103.22); 2.497(76.02); 2.464(15.81); 2.329(0.61); 0(43.75) |
| 574 | 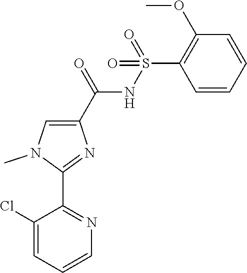 [SMILES: COc1ccccc1S(=O)(=O)NC(=O)c1cn(C)c(-c2ncccc2Cl)n1] | Example 574: ¹H-NMR(400.0 MHz, d₆-DMSO): δ = 8.69(2.07); 8.687(2.17); 8.678(2.2); 8.675(2.19); 8.178(2.27); 8.175(2.41); 8.158(3.36); 7.907(1.73); 7.903(1.82); 7.887(1.92); 7.883(1.85); 7.682(0.7); 7.664(1.39); 7.646(0.81); 7.622(1.77); 7.61(1.73); 7.601(1.67); 7.589(1.61); 7.237(1.95); 7.216(1.78); 7.165(1.1); 7.146(1.94); 7.126(0.97); 3.846(16); 3.671(15.7); 3.325(11.31); 2.67(0.61); 2.505(89.99); 2.501(115.19); 2.497 (87.58); 2.327(0.65); 0.145(0.39); −0.001(87.42); −0.151(0.41) |
| 575 | 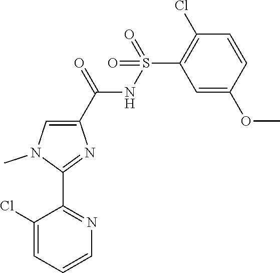 | Example 575: ¹H-NMR(400.0 MHz, d₆-DMSO): δ = 8.693(2.11); 8.69(2.12); 8.682(2.19); 8.679(2.03); 8.2(4.37); 8.177(2.04); 8.175(1.97); 8.156(2.15); 8.154(2.04); 7.628(1.68); 7.611(3.93); 7.608(3.6); 7.604(3.6); 7.596(1.84); 7.579(2.49); 7.557(2.87); 7.296(1.59); 7.288(1.51); 7.274(1.4); 7.266(1.29); 3.856(16); 3.67(14.34); 2.502(48.89); 0(40.98) |
| 576 | 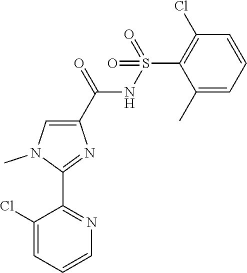 [SMILES: Cc1cccc(Cl)c1S(=O)(=O)NC(=O)c1cn(C)c(-c2ncccc2Cl)n1] | Example 576: ¹H-NMR(400.0 MHz, d₆-DMSO): δ = 8.688(2.11); 8.685(2.18); 8.677(2.26); 8.673(2.14); 8.182(1.36); 8.172 (2.47); 8.168(2.32); 8.151(2.29); 8.148(2.14); 7.621(1.75); 7.61(1.73); 7.6 (1.66); 7.589(1.62); 7.514(1.18); 7.497(3.1); 7.419(1.1); 7.404(0.93); 3.665 (16); 3.546(0.32); 3.483(0.47); 3.462(0.5); 3.402(0.63); 3.338(0.68); 2.745 (14.33); 2.675(0.64); 2.67(0.84); 2.666(0.65); 2.506(114.29); 2.501 (150.33); 2.497(109.29); 2.328(0.87); 2.074(0.78); 0.146(0.7); 0.008(7.34); 0(161.74); −0.008(7.76); −0.15(0.69) |
| 577 | 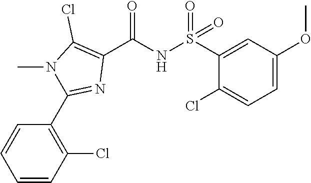 [SMILES: COc1ccc(Cl)c(S(=O)(=O)NC(=O)c2nc(-c3ccccc3Cl)n(C)c2Cl)c1] | Example 577: ¹H-NMR(400.0 MHz, d₆-DMSO): δ = 8.313(0.33); 7.696(1.13); 7.689(0.35); 7.678(2.16); 7.674(1.92); 7.653 (0.83); 7.648(1.18); 7.631(2.3); 7.619(1.71); 7.615(2.33); 7.614(2.35); 7.606 (3.24); 7.598(3.27); 7.576(1.25); 7.561(1.57); 7.558(1.95); 7.541(1.63); 7.524(0.63); 7.521(0.64); 7.293(0.76); 7.286(0.81); 7.272(0.69); 7.264 (0.69); 3.86(16); 3.416(14.69); 3.345(1.1); 3.235(0.64); 3.186(0.41); 2.675 (0.58); 2.67(0.81); 2.666(0.62); 2.523(2.12); 2.51(45.95); 2.506(95.29); 2.501(127.49); 2.496(94.12); 2.492(46.74); 2.332(0.57); 2.328(0.78); 2.323 (0.6); 0.146(0.53); 0.008(4.56); 0(125.49); −0.008(5.23); −0.15(0.52) |
| 578 | 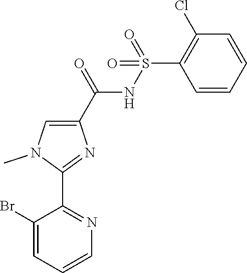 [SMILES: Cn1cc(C(=O)NS(=O)(=O)c2ccccc2Cl)nc1-c1ncccc1Br] | Example 578: ¹H-NMR(400.0 MHz, d₆-DMSO): δ = 8.724(2.11); 8.72(2.22); 8.712(2.27); 8.709(2.19); 8.319(2.03); 8.316(2.02); 8.298(2.21); 8.295(2.08); 8.161(2.38); 8.157(2.13); 8.141(1.82); 8.137 (1.86); 7.712(0.33); 7.694(1); 7.675(1.3); 7.662(2.06); 7.645(0.81); 7.624 (0.93); 7.604(1.32); 7.587(0.61); 7.541(1.89); 7.529(1.82); 7.52(1.78); 7.508(1.76); 3.797(0.34); 3.663(0.41); 3.622(16); 2.671(0.4); 2.51(24.84); 2.506(47.6); 2.502(61.45); 2.497(45.02); 2.493(22.29); 2.328(0.36); 2.075 (0.71); 0(4.28) |
| 579 | 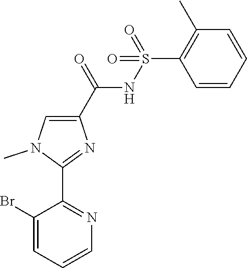 [SMILES: Cc1ccccc1S(=O)(=O)NC(=O)c1cn(C)c(-c2ncccc2Br)n1] | Example 579: ¹H-NMR(400.0 MHz, d₆-DMSO): δ = 8.72(2.16); 8.717(2.19); 8.709(2.28); 8.706(2.14); 8.316(2.1); 8.313(2.03); 8.296(2.25); 8.293(2.08); 8.102(1.49); 8.026(1.67); 8.008(1.78); 7.581 (0.59); 7.563(1.31); 7.538(2.14); 7.526(1.84); 7.518(1.75); 7.506(1.7); 7.451 (0.94); 7.432(1.48); 7.413(0.75); 7.396(1.59); 7.377(1.3); 5.757(10.83); 3.646(0.33); 3.604(16); 3.508(0.7); 3.338(5.31); 2.675(0.7); 2.671(0.91); 2.666(0.7); 2.608(13.38); 2.506(115.42); 2.502(148.36); 2.497(111.56); 2.328(0.87); 2.324(0.66); 0(8.37) |
| 580 | 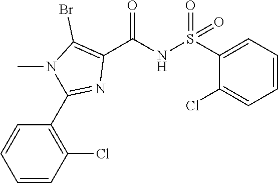 [SMILES: Cn1c(-c2ccccc2Cl)nc(C(=O)NS(=O)(=O)c2ccccc2Cl)c1Br] | Example 580: ¹H-NMR(400.0 MHz, d₆-DMSO): δ = 8.314(0.32); 8.164(5.42); 8.16(5.66); 8.144(6.02); 8.14(6.03); 7.734(1.39); 7.731(1.48); 7.714 (4.47); 7.697(9.19); 7.678(16); 7.661(3.82); 7.652(3.9); 7.648(5.05); 7.64(8.29); 7.635(9.33); 7.63(8.23); 7.621(13.02); 7.618(11.7); 7.613(6.64); 7.604(3.02); 7.6(2.56); 7.563(4.9); 7.56(4.8); 7.542(5.66); 7.526(2.3); 7.523(2.18); 3.606(0.67); 3.541(0.59); 3.43(51.54); 3.249(0.94); 2.675(0.94); 2.67(1.23); 2.506(157.77); 2.501(199.45); 2.497(148.26); 2.328 (1.32); 2.073(2.75); 0.146(0.75); 0(168.84); −0.15(0.8) |
| 581 | 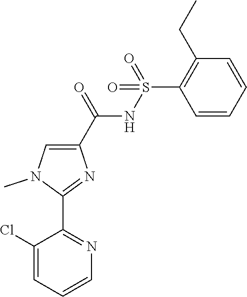 [SMILES: CCc1ccccc1S(=O)(=O)NC(=O)c1cn(C)c(-c2ncccc2Cl)n1] | Example 581: ¹H-NMR(400.0 MHz, d₆-DMSO): δ = 8.688(1.94); 8.685(2.18); 8.677(2.09); 8.674(2.15); 8.175(1.93); 8.172 (2.05); 8.155(2.16); 8.151(2.15); 8.122(4.19); 8.027(1.92); 8.007(1.94); 7.635(0.8); 7.625(2.16); 7.613(2.71); 7.604(2.1); 7.592(2); 7.455(2.31); 7.449(1.61); 7.435(1.95); 7.43(2.1); 7.411(0.86); 3.646(16); 3.466(0.5); 3.408(0.68); 3.34(0.88); 3.078(1.15); 3.06(3.37); 3.041(3.45); 3.022(1.18); 2.675(0.4); 2.67(0.55); 2.506(72.38); 2.501(96.65); 2.497(74.15); 2.332 (0.43); 2.328(0.59); 1.18(3.95); 1.161(8.46); 1.142(3.86); 0(5.75) |
| 582 | 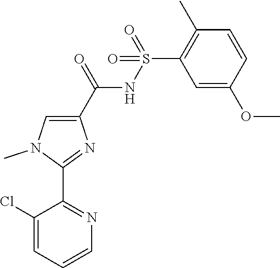 | Example 582: ¹H-NMR(400.0 MHz, d₆-DMSO): δ = 8.69(1.68); 8.687(1.88); 8.679(1.82); 8.676(1.87); 8.177(1.64); 8.173(1.77); 8.156(1.83); 8.153(1.88); 8.136(4.64); 7.626(1.67); 7.614(1.61); 7.605 (1.54); 7.594(1.55); 7.535(2.58); 7.528(2.71); 7.32(1.68); 7.299(2.18); 7.176 (1.43); 7.169(1.4); 7.155(1.11); 7.148(1.09); 3.805(16); 3.652(13.22); 2.52 (12.75); 2.506(29.93); 2.502(39.59); 2.497(30.21); 0(2.53) |
| 583 | 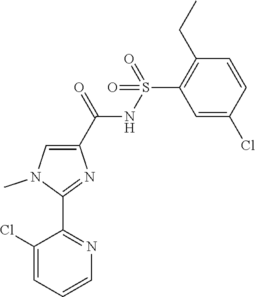 [SMILES: CCc1ccc(Cl)cc1S(=O)(=O)NC(=O)c1cn(C)c(-c2ncccc2Cl)n1] | Example 583: ¹H-NMR(400.0 MHz, d₆-DMSO): δ = 8.693(2.02); 8.69(2.31); 8.682(2.2); 8.678(2.31); 8.181(2.02); 8.178(2.21); 8.16(2.25); 8.157(2.37); 8.136(4.14); 7.98(3.33); 7.974(3.52); 7.71(1.34); 7.705(1.33); 7.69(1.66); 7.684(1.61); 7.633(1.95); 7.621(1.87); 7.612(1.82); 7.601(1.78); 7.498(2.72); 7.478(2.27); 5.756(1.29); 3.652(16); 3.186(0.34); 3.06(1.05); 3.041(3.27); 3.022(3.32); 3.004(1.11); 2.671(0.38); 2.506 (45.86); 2.502(60.39); 2.498(45.65); 2.329(0.35); 1.16(3.94); 1.142(8.41); 1.123(3.78); 0.008(2.13); 0(48.18); −0.008(2.57) |
| 584 | 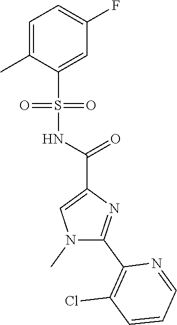 [SMILES: Cc1ccc(F)cc1S(=O)(=O)NC(=O)c1cn(C)c(-c2ncccc2Cl)n1] | Example 584: ¹H-NMR(400.0 MHz, d₆-DMSO): δ = 8.694(2.17); 8.691(2.17); 8.682(2.29); 8.679(2.15); 8.181(2.15); 8.178 (2.07); 8.161(2.41); 8.158(2.31); 8.146(5.38); 7.777(1.35); 7.772(1.53); 7.751(1.52); 7.632(1.86); 7.621(1.82); 7.612(1.77); 7.6(1.72); 7.479(1.51); 7.469(2.42); 7.454(3.56); 3.652(16); 2.671(0.33); 2.575(12.43); 2.502 (62.16); 2.498(48.67); 2.329(0.41); 0(3.33) |
| 585 | 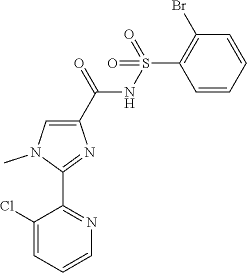 [SMILES: Cn1cc(C(=O)NS(=O)(=O)c2ccccc2Br)nc1-c1ncccc1Cl] | Example 585: ¹H-NMR(400.0 MHz, d₆-DMSO): δ = 8.692(2); 8.689(2.16); 8.681(2.15); 8.677(2.17); 8.208(2.97); 8.197(1.89); 8.193(1.87); 8.177(3.61); 8.173(3.85); 8.156(2.21); 8.152(2.17); 7.857(1.5); 7.854(1.58); 7.837(1.82); 7.835(1.84); 7.675(0.7); 7.659(1.62); 7.656(1.59); 7.64(1.21); 7.637(1.14); 7.627(2.1); 7.615(2.75); 7.606(2.07); 7.594(3.08); 7.579(0.57); 7.575(0.52); 3.671(16); 3.491(0.38); 3.467(0.33); 3.399(0.32); 2.675(0.54); 2.67(0.72); 2.666(0.53); 2.524(2.2); 2.51(48.2); 2.506(97.04); 2.502(129.19); 2.497(96.22); 2.493(48.7); 2.333(0.59); 2.328(0.81); 2.324 (0.62); 2.074(1.05); 0.146(0.51); 0.008(4.53); 0(117.32); −0.008(5.38); −0.15(0.53) |
| 586 | 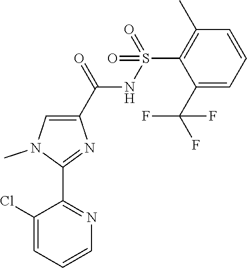 [SMILES: Cc1cccc(C(F)(F)F)c1S(=O)(=O)NC(=O)c1cn(C)c(-c2ncccc2Cl)n1] | Example 586: ¹H-NMR(400.0 MHz, d₆-DMSO): δ = 8.695(1.99); 8.692(2.08); 8.683(2.09); 8.315(0.55); 8.177(1.86); 8.159 (2.15); 8.116(0.77); 7.857(1.04); 7.71(2.17); 7.632(1.49); 7.62(1.44); 7.611 (1.37); 7.6(1.3); 3.657(16); 3.388(5.21); 3.048(0.42); 2.745(14.85); 2.67 (2.21); 2.506(289.63); 2.501(378.8); 2.497(284.15); 2.328(2.3); 2.074 (0.34); 0.146(1.5); 0.008(13.24); 0(318.93); −0.008(15.51); −0.15(1.52) |
| 587 | 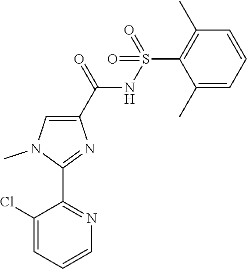 [SMILES: Cc1cccc(C)c1S(=O)(=O)NC(=O)c1cn(C)c(-c2ncccc2Cl)n1] | Example 587: ¹H-NMR(400.0 MHz, d₆-DMSO): δ = 8.688(1.22); 8.685(1.29); 8.676(1.29); 8.673(1.28); 8.171(1.18); 8.168 (1.22); 8.15(1.33); 8.147(1.31); 8.129(2.88); 7.622(1.12); 7.61(1.09); 7.601 (1.06); 7.59(1.02); 7.414(0.62); 7.395(1.31); 7.376(0.96); 7.239(2.73); 7.22 (2.2); 5.756(1.25); 3.649(9.31); 2.681(16); 2.506(21.88); 2.502(28.16); 2.498(21.7); 0(23.37) |
| 588 | 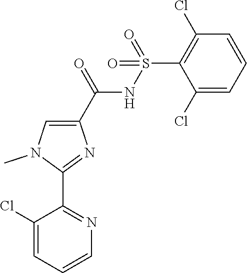 [SMILES: Cn1cc(C(=O)NS(=O)(=O)c2c(Cl)cccc2Cl)nc1-c1ncccc1Cl] | Example 588: ¹H-NMR(400.0 MHz, d₆-DMSO): δ = 8.698(2.21); 8.688(2.25); 8.196(4.2); 8.185(2.52); 8.183(2.48); 8.164(2.38); 8.162(2.39); 7.66(3.04); 7.642(7.75); 7.628(2.01); 7.618(1.86); 7.603(2.88); 7.586(1.69); 7.58(1.43); 7.563(0.89); 3.674(16); 2.671(0.36); 2.506(51.48); 2.502(67.55); 2.498(51.81); 2.328(0.39); 0.146(0.33); 0.008(3.36); 0(69.17); −0.15(0.33) |
| 589 | 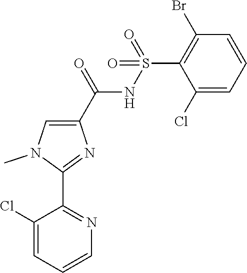 [SMILES: Cn1cc(C(=O)NS(=O)(=O)c2c(Cl)cccc2Br)nc1-c1ncccc1Cl] | Example 589: ¹H-NMR(400.0 MHz, d₆-DMSO): δ = 8.699(2.41); 8.697(2.33); 8.688(2.54); 8.686(2.35); 8.197(4.02); 8.184 (2.61); 8.183(2.42); 8.164(2.59); 7.872(2.15); 7.852(2.42); 7.691(2.07); 7.671(2.55); 7.639(2); 7.627(1.83); 7.618(1.75); 7.606(1.7); 7.492(1.52); 7.472(2.64); 7.452(1.2); 3.676(16); 2.501(69.94); 2.328(0.55); 2.074(0.44); −0.001(59.86); −0.15(0.4) |
| 590 | 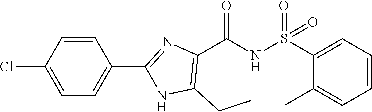 [SMILES: CCc1[nH]c(-c2ccc(Cl)cc2)nc1C(=O)NS(=O)(=O)c1ccccc1C] | Example 590: ¹H-NMR(400.0 MHz, d₆-DMSO): δ = 13.026(0.93); 8.069(3.63); 8.048(4.15); 8.038(2.74); 8.017(2.39); 7.914 (0.38); 7.893(0.44); 7.587(4.73); 7.565(5.41); 7.542(1.46); 7.453(1.12); 7.434(1.8); 7.415(0.92); 7.397(2.19); 7.377(2.12); 7.345(0.55); 4.461(0.42); 4.446(0.41); 3.638(0.51); 3.628(0.55); 3.46(0.94); 3.437(0.96); 3.382(0.93); 3.366(0.9); 3.274(0.62); 3.185(0.47); 2.851(1.09); 2.833(2.99); 2.814(3.07); 2.795(1.19); 2.671(0.88); 2.632(16); 2.501(125.11); 2.328(0.79); 2.073 |
| | | (1.19); 1.148(4.45); 1.129(9.37); 1.11(4.37); 0.146(0.43); 0(85.13); −0.15 |
| | | (0.43) |
| 591 | 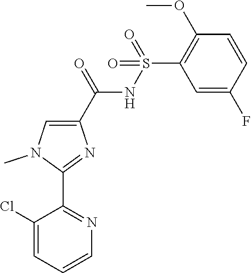 [SMILES: COc1ccc(F)cc1S(=O)(=O)NC(=O)c1cn(C)c(-c2ncccc2Cl)n1] | Example 591: ¹H-NMR(400.0 MHz, d₆-DMSO): δ = 8.69(2.16); 8.688(2.15); 8.679(2.27); 8.676(2.14); 8.184(3.75); 8.179(3.11); 8.158(2.35); 8.155(2.2); 7.672(1.37); 7.664(1.64); 7.652(1.46); 7.644(1.6); 7.624(1.8); 7.612(1.79); 7.603(1.75); 7.592(2.13); 7.572(1.25); 7.565(1.08); 7.551(0.81); 7.543(0.64); 7.293(1.32); 7.283(1.41); 7.27(1.2); 7.26(1.14); 3.842(16); 3.672(15.2); 3.399(0.33); 3.386(0.34); 3.346(0.35); 3.31(0.35); 2.502(49.61); 2.328(0.32); 2.074(0.61); 0(36.8) |
| 592 | 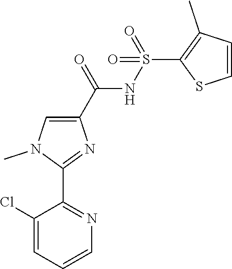 [SMILES: Cc1ccsc1S(=O)(=O)NC(=O)c1cn(C)c(-c2ncccc2Cl)n1] | Example 592: ¹H-NMR(400.0 MHz, d₆-DMSO): δ = 8.686(2.09); 8.683(2.18); 8.675(2.16); 8.672(2.12); 8.171(2.02); 8.168 (2.05); 8.15(2.22); 8.148(2.18); 8.133(0.43); 8.111(1.73); 7.849(1.17); 7.838 (1.18); 7.619(1.75); 7.608(1.74); 7.599(1.66); 7.587(1.58); 7.02(2.13); 7.008 (2.06); 3.652(16); 3.472(0.34); 3.181(0.59); 2.671(0.43); 2.506(53.34); 2.502(68.91); 2.454(14.86); 2.328(0.41); 2.074(0.35); 0.146(0.33); 0(67.52); −0.15(0.33) |
| 593 | 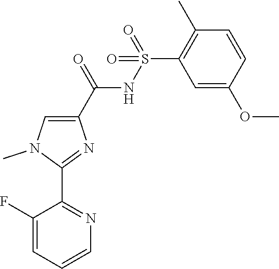 | Example 593: ¹H-NMR(400.0 MHz, d₆-DMSO): δ = 8.576(1.52); 8.565(1.58); 8.169(4.12); 7.968(0.78); 7.945(1.34); 7.923 (0.82); 7.921(0.91); 7.659(0.76); 7.648(1.3); 7.637(1.37); 7.626(1.08); 7.616(0.62); 7.537(2.46); 7.53(2.63); 7.318(1.56); 7.296(2.07); 7.174(1.35); 7.167(1.34); 7.153(1.04); 7.146(1.03); 3.814(13.2); 3.805(16); 2.522(11.8); 2.506(29.44); 2.502(39.52); 2.498(30.39); 0.008(1.42); 0(35.34) |
| 594 | 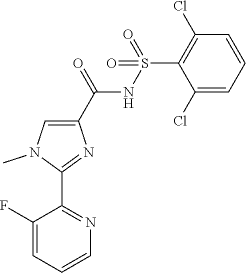 [SMILES: Cn1cc(C(=O)NS(=O)(=O)c2c(Cl)cccc2Cl)nc1-c1ncccc1F] | Example 594: ¹H-NMR(400.0 MHz, d₆-DMSO): δ = 8.587(1.91); 8.579(1.27); 8.576(1.97); 8.234(5); 7.982(0.93); 7.979(0.98); 7.961(1.17); 7.957(1.69); 7.953(1.16); 7.935(1.08); 7.932(1.13); 7.675 (1.06); 7.664(4.03); 7.66(3.47); 7.653(2.09); 7.642(8.45); 7.632(1.05); 7.603 (2.78); 7.586(1.76); 7.58(1.4); 7.563(0.95); 3.832(16); 2.671(0.39); 2.506 (52.97); 2.502(70.87); 2.497(53.07); 2.329(0.41); 2.324(0.32); 0.008(2.54); 0(64.64); −0.008(3.1) |
| 595 | 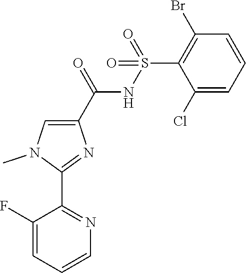 [SMILES: Cn1cc(C(=O)NS(=O)(=O)c2c(Cl)cccc2Br)nc1-c1ncccc1F] | Example 595: ¹H-NMR(400.0 MHz, d₆-DMSO): δ = 8.588(2.08); 8.58(1.53); 8.576(2.16); 8.24(4.99); 7.983(0.97); 7.98(1.07); 7.958(1.83); 7.936(1.16); 7.933(1.19); 7.875(1.88); 7.872(2.11); 7.855(2.2); 7.852(2.32); 7.693(1.85); 7.69(2.04); 7.673(2.82); 7.67(2.66); 7.665(2.19); 7.654(1.87); 7.643(1.93); 7.633(0.88); 7.494(1.83); 7.474(3.16); 7.454 (1.43); 3.835(16); 2.506(37.32); 2.502(49.31); 2.498(38.35); 0.008(2); 0(46.15) |
| 596 | 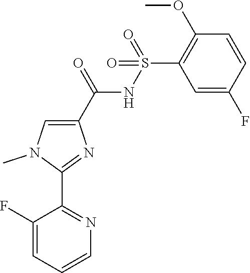 [SMILES: COc1ccc(F)cc1S(=O)(=O)NC(=O)c1cn(C)c(-c2ncccc2F)n1] | Example 596: ¹H-NMR(400.0 MHz, d₆-DMSO): δ = 8.577(1.66); 8.565(1.75); 8.217(2.68); 7.971(0.87); 7.948(1.39); 7.926(0.9); 7.923(0.98); 7.674(1.21); 7.666(1.47); 7.654(1.53); 7.646(2.68); 7.636 (1.53); 7.626(1.16); 7.615(0.7); 7.594(0.52); 7.586(0.46); 7.572(0.93); 7.564 (0.81); 7.551(0.64); 7.543(0.52); 7.292(1.11); 7.282(1.18); 7.269(0.99); 7.259(0.97); 3.843(16); 3.838(15.77); 3.388(0.47); 3.347(0.48); 2.671(0.35); 2.506(44.56); 2.502(60.07); 2.497(45.86); 2.328(0.35); 0.008(1.93); 0 (51.24); −0.008(2.72) |
| 597 | 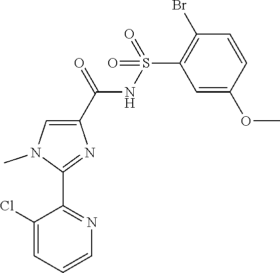 | Example 597: ¹H-NMR(400.0 MHz, d₆-DMSO): δ = 8.694(1.75); 8.691(1.97); 8.682(1.87); 8.679(1.95); 8.212(4.54); 8.178 (1.73); 8.175(1.9); 8.158(1.9); 8.154(1.99); 7.742(2.71); 7.72(2.99); 7.657 (2.89); 7.649(3.05); 7.629(1.72); 7.617(1.66); 7.608(1.6); 7.597(1.59); 7.215 (1.49); 7.208(1.46); 7.193(1.4); 7.186(1.36); 3.852(16); 3.675(13.86); 2.506 (30.17); 2.502(40.44); 2.498(31.28); 0.008(1.44); 0(35.92) |
| 598 | 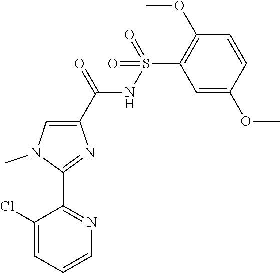 | Example 598: ¹H-NMR(400.0 MHz, d₆-DMSO): δ = 8.692(1.3); 8.688(1.47); 8.68(1.41); 8.677(1.47); 8.177(3.44); 8.16(1.5); 8.156(1.51); 7.624(1.3); 7.612(1.27); 7.603(1.22); 7.592(1.21); 7.387(2.05); 7.379(2.33); 7.273(0.7); 7.265(0.61); 7.25(1.2); 7.242(1.13); 7.193(2.14); 7.17(1.21); 3.79(16); 3.787(15.28); 3.675(10.23); 3.324(0.59); 2.506(28.04); 2.502(37.34); 2.497(28.21); 2.074(1.44); 0.008(1.49); 0(35.59); −0.008 (1.75) |
| 599 | 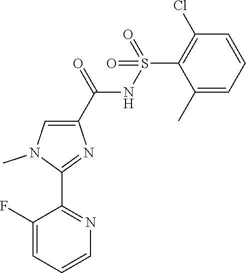 [SMILES: Cc1cccc(Cl)c1S(=O)(=O)NC(=O)c1cn(C)c(-c2ncccc2F)n1] | Example 599: ¹H-NMR(400.0 MHz, d₆-DMSO): δ = 8.575(1.9); 8.563(1.94); 8.223(2.71); 7.966(0.96); 7.964(0.91); 7.942(1.61); 7.919(1.09); 7.916(1.04); 7.657(0.91); 7.646(1.55); 7.635(1.65); 7.625 (1.28); 7.614(0.75); 7.536(0.41); 7.516(1.62); 7.499(4.11); 7.482(0.66); 7.425(1.39); 7.42(1.33); 7.408(1); 7.403(0.96); 3.826(16); 3.646(0.42); 3.622(0.38); 3.533(0.42); 3.509(0.41); 3.45(0.4); 2.748(14.19); 2.671(0.44); 2.506(55.36); 2.502(72.07); 2.498(53.51); 2.329(0.44); 2.074(2.85); 0.008 (2.39); 0(55.26); −0.008(2.68) |
| 600 | 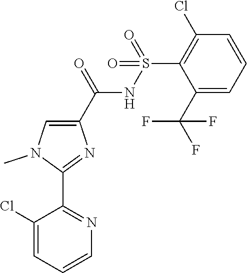 [SMILES: Cn1cc(C(=O)NS(=O)(=O)c2c(Cl)cccc2C(F)(F)F)nc1-c1ncccc1Cl] | Example 600: ¹H-NMR(400.0 MHz, d₆-DMSO): δ = 8.706(1.99); 8.703(2.16); 8.695(2.18); 8.692(2.18); 8.187(5.05); 8.172 (2.33); 8.169(2.23); 8.036(1.71); 8.017(2.08); 7.967(1.74); 7.948(2.3); 7.831 (1.33); 7.811(2.07); 7.791(0.88); 7.648(1.86); 7.636(1.8); 7.627(1.74); 7.616 (1.72); 4.213(0.37); 4.025(0.49); 4(0.48); 3.856(0.4); 3.828(0.33); 3.679 (16); 2.67(0.54); 2.506(72.36); 2.501(94.43); 2.497(72.51); 2.328(0.59); 2.074(1.87); 0(66.22); −0.15(0.33) |
| 601 | 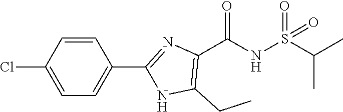 [SMILES: CCc1[nH]c(-c2ccc(Cl)cc2)nc1C(=O)NS(=O)(=O)C(C)C] | Example 601: ¹H-NMR(400.0 MHz, d₆-DMSO): δ = 13.036(1.41); 8.313(0.81); 8.132(0.73); 8.056(4.97); 8.035(5.54); 7.577 (5.03); 7.555(4.72); 7.384(1.29); 7.378(10.85); 7.373(3.84); 7.361(4.51); 7.356(16); 7.35(2.22); 7.27(12.58); 7.248(8.47); 6.537(1.76); 6.521(3.51); 6.506(1.77); 4.212(10.4); 4.196(10.25); 3.816(0.5); 3.798(1.18); 3.782 (1.57); 3.765(1.17); 3.748(0.52); 3.32(24.71); 3.174(0.62); 3.163(0.58); 2.976(1.06); 2.958(2.99); 2.939(3.1); 2.92(1.17); 2.675(1.62); 2.67(2.29); |
| | | 2.666(1.67); 2.661(0.85); 2.524(6.18); 2.51(130.94); 2.506(270.67); 2.501 |
| | | (360.32); 2.497(259.59); 2.492(124.42); 2.337(0.75); 2.332(1.59); 2.328 |
| | | (2.17); 2.323(1.57); 1.326(14.71); 1.309(14.67); 1.262(0.44); 1.244(3.87); |
| | | 1.226(7.74); 1.207(3.73); 0.146(1.2); 0.008(9.66); 0(265.56); −0.008(9.67); |
| | | −0.15(1.19) |
| 602 | 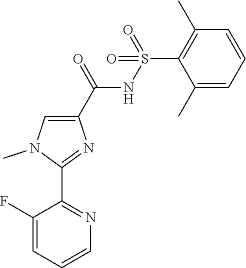 [SMILES: Cc1cccc(C)c1S(=O)(=O)NC(=O)c1cn(C)c(-c2ncccc2F)n1] | Example 602: ¹H-NMR(400.0 MHz, d₆-DMSO): δ = 8.573(1.11); 8.565(0.76); 8.562(1.15); 8.164(1.73); 7.965(0.54); 7.963 (0.54); 7.944(0.69); 7.941(0.97); 7.918(0.63); 7.916(0.62); 7.655(0.53); 7.644(0.93); 7.634(0.97); 7.623(0.78); 7.612(0.45); 7.414(0.49); 7.394 (1.05); 7.376(0.77); 7.238(2.32); 7.219(1.87); 3.81(9.66); 3.41(0.46); 3.337 (0.64); 2.682(16); 2.524(1); 2.51(20.46); 2.506(41.16); 2.502(54.54); 2.497 (40.45); 2.493(20.54); 2.328(0.33); 2.074(1.64); 0.008(1.53); 0(38.74); −0.008(1.73) |
| 603 | 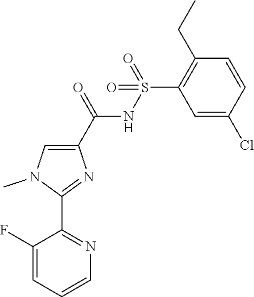 [SMILES: CCc1ccc(Cl)cc1S(=O)(=O)NC(=O)c1cn(C)c(-c2ncccc2F)n1] | Example 603: ¹H-NMR(400.0 MHz, d₆-DMSO): δ = 8.579(2.02); 8.567(2.09); 8.168(2.27); 7.979(3.64); 7.974(3.95); 7.951 (1.75); 7.929(1.17); 7.705(1.15); 7.685(1.42); 7.665(0.92); 7.655(1.53); 7.644(1.52); 7.634(1.27); 7.622(0.72); 7.496(2.02); 7.476(1.71); 3.814(16); 3.762(0.54); 3.673(0.65); 3.633(0.73); 3.596(0.7); 3.57(0.71); 3.514(0.69); 3.414(0.56); 3.375(0.5); 3.34(0.47); 3.329(0.43); 3.238(0.33); 3.06(1.23); 3.041(3.45); 3.022(3.5); 3.003(1.24); 2.671(0.92); 2.506(123.85); 2.502 (153.59); 2.498(115.93); 2.328(0.9); 1.164(3.89); 1.145(8.13); 1.126(3.7); 0.146(0.42); 0(90.64); −0.15(0.42) |
| 604 | 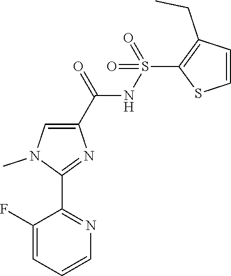 [SMILES: CCc1ccsc1S(=O)(=O)NC(=O)c1cn(C)c(-c2ncccc2F)n1] | Example 604: ¹H-NMR(400.0 MHz, d₆-DMSO): δ = 8.58(1.11); 8.576(1.86); 8.568(1.25); 8.565(1.91); 8.183(4.41); 7.972(0.91); 7.97(0.93); 7.951(1.16); 7.948(1.63); 7.944(1.08); 7.926(1.09); 7.923(1.09); 7.908(2.43); 7.896(2.51); 7.66(0.97); 7.649(1.58); 7.638(1.7); 7.628(1.3); 7.617(0.78); 7.118(3.01); 7.105(2.96); 5.756(0.98); 3.819(16); 3.639(0.37); 3.603(0.35); 3.583(0.37); 3.549(0.39); 3.536(0.39); 3.533(0.39); 3.522 (0.39); 3.502(0.4); 3.484(0.4); 3.459(0.4); 3.425(0.39); 3.386(0.37); 3.38 (0.36); 3.358(0.34); 2.956(1.19); 2.938(3.67); 2.919(3.75); 2.9(1.26); 2.671 (0.41); 2.511(26.11); 2.506(52.22); 2.502(68.88); 2.498(50.57); 2.329(0.41); 1.159(4.37); 1.141(9.29); 1.122(4.21); 0.008(2.13); 0(52.38); −0.008(2.25) |
| 605 | 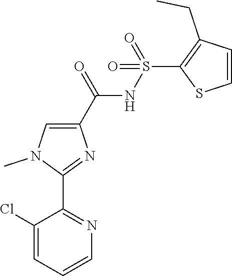 [SMILES: CCc1ccsc1S(=O)(=O)NC(=O)c1cn(C)c(-c2ncccc2Cl)n1] | Example 605: ¹H-NMR(400.0 MHz, d₆-DMSO): δ = 8.689(1.93); 8.686(2.17); 8.678(2.07); 8.674(2.18); 8.176(1.89); 8.173 (2.05); 8.156(2.39); 8.152(2.92); 8.147(3.53); 7.904(1.86); 7.892(1.92); 7.626(1.87); 7.614(1.81); 7.605(1.76); 7.593(1.74); 7.117(2.72); 7.104 (2.65); 3.654(16); 3.474(0.52); 3.378(0.65); 3.353(0.65); 3.217(0.38); 2.952 (1.17); 2.934(3.65); 2.915(3.73); 2.896(1.23); 2.675(0.47); 2.67(0.64); 2.666 (0.51); 2.506(83.39); 2.501(112.17); 2.497(85.19); 2.332(0.48); 2.328(0.66); 2.323(0.52); 2.074(0.54); 1.155(4.29); 1.136(9.24); 1.117(4.21); 0.146 (0.34); 0.008(3.1); 0(80.07); −0.008(3.95); −0.15(0.37) |
| 606 | 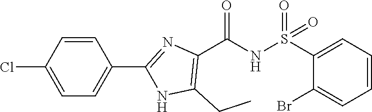 [SMILES: CCc1[nH]c(-c2ccc(Cl)cc2)nc1C(=O)NS(=O)(=O)c1ccccc1Br] | Example 606: ¹H-NMR(400.0 MHz, d₆-DMSO): δ = 13.237(0.71); 8.19(3.57); 8.186(3.82); 8.171(3.98); 8.167(3.99); 8.069 (6.42); 8.048(7.18); 7.836(3.39); 7.833(3.62); 7.816(4.1); 7.813(4.16); 7.657 (1.55); 7.654(1.71); 7.638(3.64); 7.636(3.62); 7.619(2.69); 7.616(2.79); 7.606(11.14); 7.602(3.8); 7.585(12.35); 7.566(3.1); 7.562(3.11); 7.547 (1.33); 7.543(1.27); 7.378(1.82); 7.374(0.68); 7.362(0.71); 7.357(2.66); 7.351(0.41); 7.271(2.1); 7.25(1.41); 6.524(0.54); 4.214(1.81); 4.199(1.8); |
| | | 2.894(1.28); 2.875(3.84); 2.856(3.95); 2.837(1.43); 2.676(0.34); 2.672 |
| | | (0.49); 2.667(0.36); 2.525(0.92); 2.52(1.5); 2.512(28.18); 2.507(60.03); |
| | | 2.503(81.13); 2.498(59.57); 2.494(29.34); 2.434(0.36); 2.334(0.37); 2.329 |
| | | (0.52); 2.325(0.4); 2.074(1.5); 1.168(7.22); 1.149(16); 1.13(7.16); 0.008 |
| | | (1.96); 0(71.46); −0.008(3.11) |
| 607 | 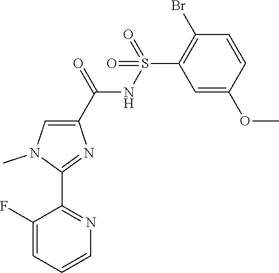 | Example 607: ¹H-NMR(400.0 MHz, d₆-DMSO): δ = 8.579(1.66); 8.568(1.74); 8.24(1.97); 7.97(0.81); 7.947(1.46); 7.925(0.94); 7.922(0.96); 7.735(2.03); 7.713(2.24); 7.662(0.98); 7.656(3.05); 7.649 (3.37); 7.641(1.6); 7.63(1.19); 7.62(0.71); 7.21(1.09); 7.202(1.06); 7.188 (1.03); 7.18(0.99); 3.852(16); 3.833(14.01); 3.668(0.37); 3.652(0.33); 3.57 (0.35); 3.558(0.36); 3.522(0.35); 3.489(0.36); 2.671(0.96); 2.666(0.72); 2.506(130.02); 2.502(170.45); 2.497(126.79); 2.332(0.77); 2.328(1.04); 2.324(0.77); 0.146(0.72); 0.008(6.79); 0(153.88); −0.008(7.02); −0.15(0.75) |
| 608 | 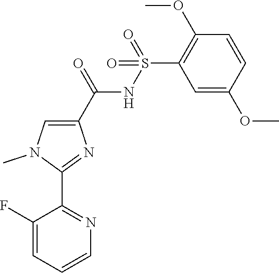 | Example 608: ¹H-NMR(400.0 MHz, d₆-DMSO): δ = 8.577(0.81); 8.569(0.56); 8.566(0.85); 8.21(1.72); 7.976(0.4); 7.973(0.43); 7.955(0.5); 7.951(0.67); 7.929(0.48); 7.926(0.48); 7.659(0.42); 7.648(0.68); 7.637(0.74); 7.627(0.56); 7.616(0.35); 7.387(1.36); 7.379(1.55); 7.274 (0.47); 7.266(0.41); 7.251(0.79); 7.244(0.76); 7.194(1.4); 7.171(0.8); 3.841 (6.86); 3.791(16); 3.321(1.66); 2.675(0.35); 2.67(0.47); 2.666(0.34); 2.523 (1.5); 2.51(29.77); 2.506(60.37); 2.501(80.45); 2.497(59.89); 2.493(30.58); 2.328(0.47); 2.324(0.35); 0.146(0.35); 0.008(2.98); 0(76.17); −0.008(3.5); −0.15(0.35) |
| 609 | 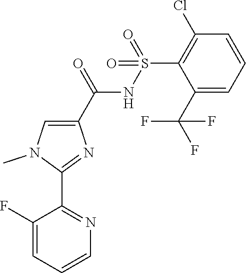 [SMILES: Cn1cc(C(=O)NS(=O)(=O)c2c(Cl)cccc2C(F)(F)F)nc1-c1ncccc1F] | Example 609: ¹H-NMR(400.0 MHz, d₆-DMSO): δ = 8.593(1.9); 8.582(1.95); 8.224(4.14); 8.037(1.64); 8.018(1.94); 7.987(0.98); 7.964(2.98); 7.962(2.8); 7.943(3.13); 7.83(1.25); 7.81(1.98); 7.79(0.83); 7.684(0.93); 7.673(1.59); 7.662(1.67); 7.652(1.33); 7.641(0.76); 3.837(16); 2.675(0.5); 2.67(0.69); 2.666(0.52); 2.506(91.7); 2.501(122.87); 2.497 (93.15); 2.333(0.52); 2.328(0.74); 2.324(0.54); 0.146(0.53); 0.008(4.75); 0(118.11); −0.008(5.95); −0.15(0.53) |
| 610 | 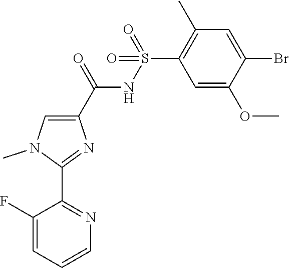 [SMILES: COc1cc(S(=O)(=O)NC(=O)c2cn(C)c(-c3ncccc3F)n2)c(C)cc1Br] | Example 610: ¹H-NMR(400.0 MHz, d₆-DMSO): δ = 8.578(1.85); 8.567(1.88); 8.169(4.03); 7.974(0.89); 7.971(0.92); 7.949 (1.63); 7.927(1.05); 7.924(1.05); 7.674(4.34); 7.663(1.11); 7.652(1.67); 7.641(1.99); 7.635(5.81); 7.62(0.79); 3.906(16); 3.814(15.21); 3.44(0.33); 3.392(0.33); 2.675(0.62); 2.671(0.79); 2.667(0.62); 2.522(16.39); 2.506 (98.54); 2.502(128.86); 2.498(95.49); 2.333(0.55); 2.328(0.74); 2.324(0.55); 2.074(1.56); 0.146(0.53); 0.008(5.04); 0(110.62); −0.008(5); −0.15(0.51) |
| 611 | 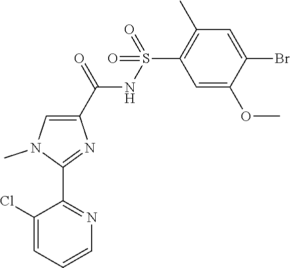 | Example 611: ¹H-NMR(400.0 MHz, d₆-DMSO): δ = 8.692(2.04); 8.689(2.28); 8.68(2.16); 8.677(2.23); 8.178(2.03); 8.175(2.19); 8.158(2.27); 8.155(2.36); 8.138(5.5); 7.677(4.91); 7.633(5.78); 7.617(1.92); 7.608(1.81); 7.597(1.73); 3.906(16); 3.654(15.34); 2.672(0.38); 2.522 (16.09); 2.506(47.91); 2.502(61.28); 2.498(48.22); 2.329(0.38); 2.075(0.49); 0(53.11) |
| 612 | 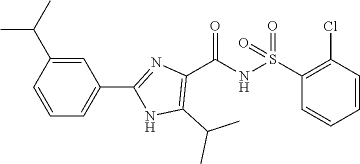 [SMILES: CC(C)c1cccc(-c2nc(C(=O)NS(=O)(=O)c3ccccc3Cl)c(C(C)C)[nH]2)c1] | Example 612: ¹H-NMR(400.0 MHz, d₆-DMSO): δ = 8.134(1.38); 8.131(1.35); 8.113(1.54); 7.942(2.26); 7.881(1.21); 7.861(1.3); 7.592(0.94); 7.577(2.96); 7.564(0.62); 7.551(0.84); 7.545(0.69); 7.531 (0.93); 7.517(0.48); 7.51(0.37); 7.444(0.78); 7.425(1.78); 7.406(1.16); 7.358(1.39); 7.339(0.85); 5.754(4.78); 3.865(0.35); 3.85(0.45); 3.835(0.37); 3.183(2.84); 3(0.35); 2.982(0.81); 2.965(1.1); 2.948(0.85); 2.931(0.36); 2.762(0.62); 2.512(6.98); 2.508(13.82); 2.504(18.19); 2.499(13.73); 1.269 (14.18); 1.252(16); 1.246(11.05); 1.229(9.49); 0(2.22) |
| 613 | 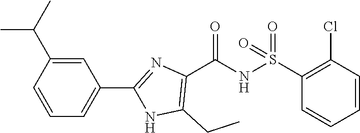 [SMILES: CCc1[nH]c(-c2cccc(C(C)C)c2)nc1C(=O)NS(=O)(=O)c1ccccc1Cl] | Example 613: ¹H-NMR(400.0 MHz, d₆-DMSO): δ = 8.13(1.75); 8.109(1.93); 7.94(2.85); 7.858(1.59); 7.839(1.68); 7.573(4.06); 7.547(1.19); 7.528(1.29); 7.514(0.66); 7.45(1.04); 7.431(2.27); 7.412(1.46); 7.366(2.03); 7.347(1.2); 3.184(1.14); 2.997(0.48); 2.98(1.14); 2.962(2.05); 2.943(2.75); 2.923(2.38); 2.905(0.94); 2.762(0.41); 2.671(0.33); 2.502 (55.57); 2.329(0.41); 1.268(15.89); 1.251(16); 1.194(3.48); 1.176(6.87); 1.157(3.36); 0(5.19) |
| 614 | 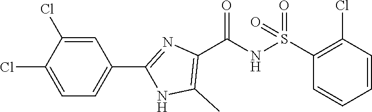 [SMILES: Cc1[nH]c(-c2ccc(Cl)c(Cl)c2)nc1C(=O)NS(=O)(=O)c1ccccc1Cl] | Example 614: ¹H-NMR(400.0 MHz, d₆-DMSO): δ = 13.28(0.34); 8.321(2.24); 8.166(1.58); 8.147(1.7); 8(1.27); 7.995(1.27); 7.978(1.56); 7.974(1.55); 7.8(2.62); 7.778(2.18); 7.712(0.38); 7.695(1.2); 7.674(1.7); 7.664(2.57); 7.648(0.86); 7.628(1); 7.626(0.94); 7.608(1.44); 7.592(0.63); 7.588(0.6); 2.503(31.24); 2.41(11.28); 2.075(16); 0(33.33) |
| 615 | 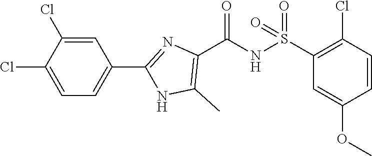 [SMILES: COc1ccc(Cl)c(S(=O)(=O)NC(=O)c2nc(-c3ccc(Cl)c(Cl)c3)[nH]c2C)c1] | Example 615: ¹H-NMR(400.0 MHz, d₆-DMSO): δ = 8.322(2.76); 7.999(1.48); 7.994(1.41); 7.978(1.79); 7.973(1.75); 7.798 (2.97); 7.777(2.46); 7.617(2.87); 7.61(3); 7.567(2.31); 7.545(2.71); 7.277 (1.43); 7.27(1.39); 7.255(1.26); 7.248(1.2); 5.754(3.94); 3.856(16); 3.186 (0.48); 2.503(34.12); 2.424(13.36); 2.33(0.33); 2.184(0.41); 1.356(2.28); 0 (3.46) |
| 616 | 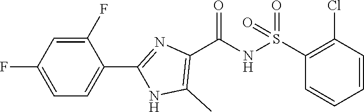 [SMILES: Cc1[nH]c(-c2ccc(F)cc2F)nc1C(=O)NS(=O)(=O)c1ccccc1Cl] | Example 616: ¹H-NMR(400.0 MHz, d₆-DMSO): δ = 12.906(0.36); 12.896(0.35); 8.314(0.47); 8.199(0.53); 8.177(1.17); 8.158 (3.17); 8.155(3.15); 8.137(2.84); 7.694(0.63); 7.675(1.52); 7.649(3.23); 7.632(1.1); 7.612(1.36); 7.592(1.86); 7.575(0.83); 7.495(0.98); 7.488(1.1); 7.471(1.25); 7.466(1.85); 7.461(1.36); 7.443(0.98); 7.438(0.97); 7.293 (1.09); 7.288(1.02); 7.272(2); 7.268(1.77); 7.251(1.03); 7.246(0.88); 5.754 (5.31); 4.091(0.34); 4.021(0.44); 3.958(0.49); 3.934(0.52); 3.914(0.54); |
| | | 3.896(0.56); 3.85(0.6); 3.841(0.61); 3.815(0.63); 3.769(0.62); 3.739(0.61); |
| | | 3.612(0.48); 3.532(0.4); 3.526(0.39); 3.514(0.38); 3.509(0.39); 3.186(0.37); |
| | | 2.77(0.35); 2.764(0.33); 2.675(0.92); 2.671(1.24); 2.666(0.96); 2.531(4.35); |
| | | 2.506(140.31); 2.502(181.14); 2.497(135.75); 2.414(16); 2.333(0.9); 2.328 |
| | | (1.2); 2.324(0.91); 1.235(0.74); 0.008(1.14); 0(19.75) |
| 617 | 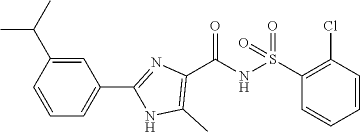 [SMILES: Cc1[nH]c(-c2cccc(C(C)C)c2)nc1C(=O)NS(=O)(=O)c1ccccc1Cl] | Example 617: ¹H-NMR(400.0 MHz, d₆-DMSO): δ = 8.14(1.52); 8.123(1.63); 7.942(2.3); 7.852(1.27); 7.833(1.37); 7.613(0.98); 7.597(3.26); 7.583(0.67); 7.569(0.94); 7.564(0.75); 7.549(1.06); 7.534(0.5); 7.528(0.44); 7.464(1.05); 7.445(2.39); 7.426(1.53); 7.379(1.78); 7.36(1.08); 4.063(0.32); 4.055(0.33); 4.037(0.33); 4.017(0.35); 3.986(0.39); 3.98(0.39); 3.976(0.39); 3.884(0.5); 3.852(0.57); 3.744(0.87); 3.635(1.57); 3.596(1.76); 3.006(0.36); 2.988(0.94); 2.971(1.28); 2.954(0.97); 2.937(0.39); 2.679 (0.36); 2.514(46.43); 2.51(60.72); 2.505(45.26); 2.487(8.82); 2.336(0.39); |
| | | 1.276(16); 1.259(15.85) |
| 618 | 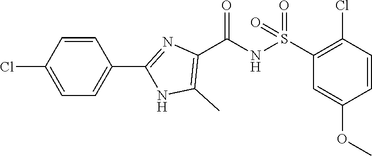 [SMILES: COc1ccc(Cl)c(S(=O)(=O)NC(=O)c2nc(-c3ccc(Cl)cc3)[nH]c2C)c1] | Example 618: ¹H-NMR(400.0 MHz, d₆-DMSO): δ = 13.358(0.45); 8.048(3.51); 8.027(3.88); 7.613(3.74); 7.606(7.31); 7.585 (4.2); 7.549(2.34); 7.527(2.62); 7.255(1.42); 7.248(1.41); 7.234(1.24); 7.226(1.19); 3.849(16); 3.809(1.11); 2.671(0.5); 2.502(84.98); 2.442 (11.28); 2.329(0.64); 2.074(3.2); 1.26(1.07); 1.242(1.07); 0(2.59) |
| 619 | 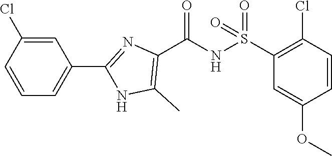 [SMILES: COc1ccc(Cl)c(S(=O)(=O)NC(=O)c2nc(-c3cccc(Cl)c3)[nH]c2C)c1] | Example 619: ¹H-NMR(400.0 MHz, d₆-DMSO): δ = 8.223(0.46); 8.204(0.46); 8.15(2.53); 8.132(0.41); 7.981(1.63); 7.962(1.74); 7.612(3.13); 7.605(3.32); 7.541(1.04); 7.533(1.29); 7.522(2.22); 7.51(1.52); 7.503(1.82); 7.484(1.78); 7.464(0.75); 7.232(0.78); 7.215(0.71); 6.989 (0.46); 6.97(0.44); 3.846(16); 3.816(0.95); 3.185(4.73); 2.784(0.46); 2.76 (1.97); 2.675(0.52); 2.671(0.67); 2.506(81.98); 2.502(106.09); 2.497 (80.51); 2.429(12.92); 2.333(0.57); 2.329(0.73); 2.074(1.09); 0.987(0.66); 0.97(0.33); 0(3.78) |
| 620 | 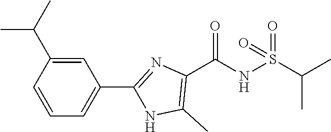 [SMILES: Cc1[nH]c(-c2cccc(C(C)C)c2)nc1C(=O)NS(=O)(=O)C(C)C] | Example 620: ¹H-NMR(400.0 MHz, d₆-DMSO): δ = 13.011(0.63); 8.314(0.56); 7.9(2.49); 7.818(1.45); 7.798(1.55); 7.409(0.84); 7.39(1.74); 7.371(1.05); 7.294(1.44); 7.274(1.06); 3.809(0.62); 3.792(0.82); 3.775(0.66); 3.758(0.33); 3.477(0.34); 3.335(2.18); 3.15(0.35); 2.99(0.44); 2.972(0.98); 2.955(1.29); 2.938(1.01); 2.921(0.46); 2.674(1.1); 2.67(1.44); 2.666(1.06); 2.506(170.16); 2.501(218.64); 2.497(164.06); 2.332(1.06); 2.328(1.4); 2.324(1.04); 2.006(0.66); 1.327(7.85); 1.31(7.85); 1.269(16); 1.251(15.91); 0.008(0.37); 0(7) |
| 621 | 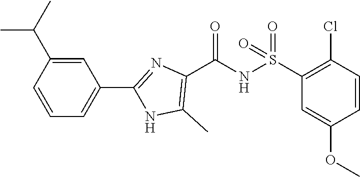 [SMILES: COc1ccc(Cl)c(S(=O)(=O)NC(=O)c2nc(-c3cccc(C(C)C)c3)[nH]c2C)c1] | Example 621: ¹H-NMR(400.0 MHz, d₆-DMSO): δ = 7.941(1.82); 7.875(1.02); 7.856(1.14); 7.609(2.05); 7.601(2.2); 7.471(0.86); 7.449(1.32); 7.429(1.2); 7.41(0.89); 7.366(1.09); 7.348(0.7); 7.161(0.69); 7.142(0.6); 5.754(6.77); 3.902(0.4); 3.89(0.35); 3.83(9.69); 3.817(1.4); 2.983(0.69); 2.966(0.93); 2.948(0.75); 2.932(0.34); 2.772(0.66); 2.503 (28.97); 1.34(0.5); 1.323(0.64); 1.3(0.43); 1.269(10.98); 1.253(16); 1.237 (7.5); 0(16.84) |
| 622 | 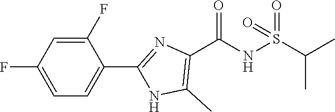 [SMILES: Cc1[nH]c(-c2ccc(F)cc2F)nc1C(=O)NS(=O)(=O)C(C)C] | Example 622: ¹H-NMR(400.0 MHz, d₆-DMSO): δ = 12.754(1.05); 8.2(0.46); 8.179(1.06); 8.161(1.08); 8.141(0.48); 7.48(0.86); 7.474(0.87); 7.452(1.63); 7.429(0.86); 7.423(0.81); 7.274(0.94); 7.27(0.9); 7.253(1.72); 7.248(1.61); 7.232(0.9); 7.227(0.8); 5.753(1.33); 3.815(0.51); 3.798(1.22); 3.78(1.64); 3.763(1.23); 3.746(0.51); 3.384(0.41); 3.336 (0.46); 3.328(0.46); 2.671(0.33); 2.52(14.13); 2.502(48.58); 2.498(37.05); 2.329(0.32); 1.324(16); 1.307(15.59); 0(46.51) |
| 623 | 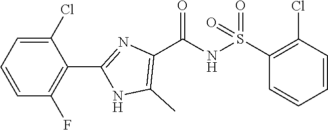 [SMILES: Cc1[nH]c(-c2c(F)cccc2Cl)nc1C(=O)NS(=O)(=O)c1ccccc1Cl] | Example 623: ¹H-NMR(400.0 MHz, d₆-DMSO): δ = 13.334(0.55); 8.313(0.38); 8.15(1.75); 8.147(1.97); 8.127(2.07); 7.693 (0.42); 7.675(1.35); 7.654(3.37); 7.648(3.17); 7.642(1.29); 7.635(2.06); 7.62(1.7); 7.615(1.43); 7.61(1.47); 7.606(1.33); 7.6(1.37); 7.59(1.67); 7.574(0.75); 7.569(0.7); 7.534(2.77); 7.514(1.83); 7.455(1.31); 7.433(2.13); 7.411(1.02); 3.977(0.38); 3.703(0.85); 3.688(0.85); 3.436(0.38); 3.433 (0.38); 2.674(0.68); 2.67(0.93); 2.505(106.13); 2.501(141.14); 2.497 (110.32); 2.418(16); 2.332(0.78); 2.328(1); 2.324(0.78); 2.073(3.15); 0 |
| | | (3.92) |
| 624 | 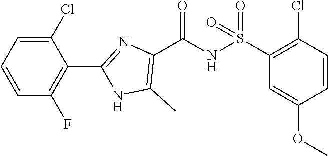 [SMILES: COc1ccc(Cl)c(S(=O)(=O)NC(=O)c2nc(-c3c(F)cccc3Cl)[nH]c2C)c1] | Example 624: ¹H-NMR(400.0 MHz, d₆-DMSO): δ = 13.387(0.36); 7.66(0.51); 7.644(0.64); 7.639(1.27); 7.624(1.34); 7.619(1.1); 7.605(3.43); 7.598(3.19); 7.552(2.24); 7.536(2.67); 7.53(3); 7.516(1.7); 7.458(1.1); 7.436(1.84); 7.414(0.88); 7.258(1.31); 7.25(1.33); 7.236(1.21); 7.228(1.15); 3.85(16); 2.506(37.23); 2.501(50.08); 2.497(40.55); 2.434 (13.01); 2.328(0.36); 2.073(0.51); 0.008(1.24); 0(24.38) |
| 625 | 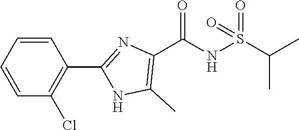 [SMILES: Cc1[nH]c(-c2ccccc2Cl)nc1C(=O)NS(=O)(=O)C(C)C] | Example 625: ¹H-NMR(400.0 MHz, d₆-DMSO): δ = 12.895(0.72); 7.831(1.56); 7.824(1.4); 7.813(1.71); 7.807(1.86); 7.611 (1.54); 7.606(1.76); 7.588(2.41); 7.513(0.53); 7.508(0.82); 7.495(1.93); 7.489(2.13); 7.483(2.19); 7.477(3.47); 7.47(1.85); 7.465(2.09); 7.461 (1.91); 7.446(0.58); 3.807(0.49); 3.79(1.19); 3.773(1.61); 3.756(1.23); 3.738(0.52); 2.518(14.73); 2.505(33.35); 2.501(43.96); 2.497(35.81); 2.073(0.45); 1.319(16); 1.302(15.72); 0(1.22) |
| 626 | 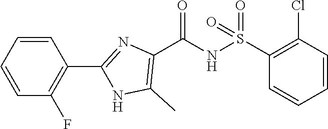 [SMILES: Cc1[nH]c(-c2ccccc2F)nc1C(=O)NS(=O)(=O)c1ccccc1Cl] | Example 626: ¹H-NMR(400.0 MHz, d₆-DMSO): δ = 12.93(0.65); 8.167(2.71); 8.164(2.87); 8.147(3.31); 8.144(3.94); 8.124 (2.24); 8.12(2.27); 8.105(1.17); 8.101(1.13); 7.707(0.64); 7.703(0.68); 7.687(2.1); 7.683(2); 7.67(3.09); 7.662(4.21); 7.658(4.67); 7.642(1.53); 7.621(1.95); 7.617(1.67); 7.602(2.43); 7.585(1.18); 7.58(1.05); 7.548 (0.59); 7.544(0.67); 7.535(0.77); 7.53(1.44); 7.526(1.4); 7.509(1.78); 7.505 (1.1); 7.496(0.97); 7.492(0.9); 7.418(1.89); 7.397(1.61); 7.39(2.13); 7.377 (2.57); 7.375(2.35); 7.371(1.67); 7.358(3.56); 7.34(1.69); 7.337(1.62); 2.671 |
| | | (0.42); 2.511(29.04); 2.507(55.28); 2.502(72.64); 2.498(54.68); 2.493 |
| | | (27.79); 2.43(16); 2.334(0.4); 2.329(0.52); 2.324(0.4); 2.074(1.77); 0(0.83) |
| 627 | 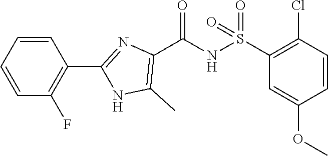 [SMILES: COc1ccc(Cl)c(S(=O)(=O)NC(=O)c2nc(-c3ccccc3F)[nH]c2C)c1] | Example 627: ¹H-NMR(400.0 MHz, d₆-DMSO): δ = 8.13(0.7); 8.127(0.75); 8.111(1.35); 8.108(1.39); 8.092(0.71); 8.088(0.69); 7.619(2.99); 7.611(3.07); 7.562(2.28); 7.553(0.59); 7.549(0.64); 7.54(2.98); 7.515(0.99); 7.501(0.54); 7.497(0.49); 7.422(1.1); 7.4(0.97); 7.394(1.22); 7.378(1.48); 7.376(1.59); 7.359(2); 7.341(0.93); 7.269(1.34); 7.261(1.31); 7.247(1.17); 7.239(1.09); 3.854(16); 2.507(23.52); 2.502(30.61); 2.498 (23.75); 2.445(9.3); 2.074(6.26) |
| 628 | 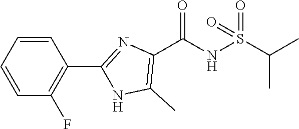 [SMILES: Cc1[nH]c(-c2ccccc2F)nc1C(=O)NS(=O)(=O)C(C)C] | Example 628: ¹H-NMR(400.0 MHz, d₆-DMSO): δ = 12.744(1.02); 8.143(0.78); 8.124(1.48); 8.106(0.8); 7.512(0.4); 7.495(1.01); 7.478(1.19); 7.463(0.64); 7.401(1.31); 7.373(1.46); 7.353(2.4); 7.334(2.47); 7.315(1.14); 3.822(0.47); 3.804(1.16); 3.787(1.57); 3.77(1.19); 3.753(0.49); 2.53(11.26); 2.502(33.64); 2.074(0.51); 1.33(16); 1.313(15.71) |

### Use Examples

Boophilus microplus—Injection Test

Solvent: dimethyl sulphoxide

To produce a suitable preparation of active compound, 10 mg of active compound are mixed with 0.5 ml of solvent and the concentrate is diluted with solvent to the desired concentration.

1 μl of the active compound solution is injected into the abdomen of 5 engorged adult female cattle ticks (Boophilus microplus). The animals are transferred into dishes and kept in a climate-controlled room.

Efficacy is assessed after 7 days by laying of fertile eggs. Eggs which are not externally visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples show an efficacy of 80% at an application rate of 20 μg/animal: 219

Lucilia cuprina Test

Solvent: dimethyl sulphoxide

To produce a suitable preparation of active compound, 10 mg of active compound are mixed with 0.5 ml of dimethyl sulphoxide, and the concentrate is diluted with water to the desired concentration.

About 20 L1 larvae of the Australian sheep blowfly (Lucilia cuprina) are transferred into a test vessel containing minced horsemeat and the active compound preparation of the desired concentration.

After 2 days, the kill in % is determined. 100% means that all the larvae have been killed; 0% means that no larvae have been killed.

In this test, for example, the following compounds from the preparation examples show an efficacy of 100% at an application rate of 100 ppm: 18, 23, 25, 27, 36, 37, 54, 56, 68, 101, 102, 107, 109, 120, 186, 218, 219, 231, 285, 341, 364, 383, 397, 401, 519, 539, 558, 572, 576, 584

In this test, for example, the following compounds from the preparation examples show an efficacy of 95% at an application rate of 100 ppm: 92, 108, 274, 336, 394

Musca domestica Test

Solvent: dimethyl sulphoxide

To produce a suitable preparation of active compound, 10 mg of active compound are mixed with 0.5 ml of dimethyl sulphoxide, and the concentrate is diluted with water to the desired concentration.

Vessels containing a sponge treated with sugar solution and the active compound preparation of the desired concentration are populated with 10 adult houseflies (Musca domestica).

After 2 days, the kill in % is determined. 100% means that all of the flies have been killed; 0% means that none of the flies have been killed.

In this test, for example, the following compounds from the preparation examples show an efficacy of 100% at an application rate of 100 ppm: 107, 151, 231, 394, 576

In this test, for example, the following compounds from the preparation examples show an efficacy of 90% at an application rate of 100 ppm: 172, 174

In this test, for example, the following compounds from the preparation examples show an efficacy of 85% at an application rate of 100 ppm: 186, 219

In this test, for example, the following compounds from the preparation examples show an efficacy of 80% at an application rate of 100 ppm: 32, 52, 68, 152, 153, 155, 185, 337

Meloidogyne incognita Test

Solvent: 125.0 parts by weight of acetone

To produce a suitable active compound preparation, 1 part by weight of active compound is mixed with the stated amount of solvent and the concentrate is diluted with water to the desired concentration.

Vessels are filled with sand, active compound solution, an egg/larvae suspension of the southern rootknot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compounds from the preparation examples show an efficacy of 100% at an application rate of 20 ppm: 1, 6, 8, 9, 33, 34, 36, 37, 38, 40, 42, 43, 44, 45, 46, 47, 49, 51, 54, 60, 61, 63, 81, 96, 103, 106, 113, 114, 140, 152, 171, 172, 173, 174, 175, 176, 178, 182, 183, 184, 188, 197, 200, 203, 205, 206, 221, 222, 227, 228, 230, 231, 238, 260, 263.264, 265, 267, 269, 270, 271, 272, 275, 276, 279, 281, 282, 285, 286, 294, 307, 309, 310, 311, 315, 329, 336, 338, 339, 341, 352, 362, 364, 380, 414, 415, 417, 429, 434, 440, 441, 443, 444, 445, 449, 450, 456, 457, 484, 496, 502, 511, 513, 517, 519, 520, 525, 535, 537, 541, 542, 544, 545, 546, 547, 560, 561, 577, 580, 581, 599, 600, 614, 616, 618, 623, 625

In this test, for example, the following compounds from the preparation examples show an efficacy of 90% at an application rate of 20 ppm: 2, 4, 10, 13, 14, 15, 16, 18, 21, 27, 29, 32, 35, 39, 41, 48, 50, 52, 55, 56, 58, 62, 64, 67, 68, 69, 71, 77, 78, 79, 82, 85, 93, 98, 99, 100, 101, 102, 104, 108, 109, 118, 119, 120, 123, 129, 134, 139, 141, 142, 149, 153, 156, 158, 164, 169, 179, 185, 186, 187, 192, 193.194, 195, 199, 202, 207, 209, 212, 216, 217, 218, 219, 232, 233, 235, 239, 243, 244, 246, 251, 253, 254, 257, 261, 262, 266, 273, 273, 274, 277, 280, 283, 284, 287, 288, 289, 291, 292, 293, 295, 296, 308, 312, 313, 314, 316, 317, 318, 319, 320, 321, 323, 324, 325, 330, 331, 334, 335, 337, 340, 343, 344, 350, 351, 353, 354, 355, 356, 357, 358, 359, 360, 361, 363, 365, 369, 371, 372, 373, 375, 376, 377, 378, 382, 384, 385, 386, 392, 393, 394, 395, 396, 397, 398, 399, 401, 403, 405, 406, 407, 408, 409, 410, 411, 419, 416, 420, 421, 422, 423, 424, 425, 426, 427, 428, 431, 435, 436, 437, 438, 439, 442, 446, 448, 453, 471, 472, 473, 475, 477, 478, 479, 480, 481, 485, 486, 487, 495, 497, 498, 499, 500, 503, 504, 505, 506, 507, 508, 510, 512, 514, 515, 516, 518, 522, 527, 528, 530, 532, 534, 536, 538, 543, 548, 550, 551, 552, 553, 554, 555, 556, 557, 558, 567, 568, 575, 579, 582, 583, 584, 586, 587, 588, 590, 593, 594, 595, 596, 597, 602, 603, 605, 613, 621, 622, 624, 626, 627

In this test, for example, the following compounds from the preparation examples show an efficacy of 80% at an application rate of 20 ppm: 225, 226

Myzus persicae—Spray Test
Solvent: 78 parts by weight of acetone
1.5 parts by weight of dimethylformamide
   Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) infested by all stages of the green peach aphid (Myzus persicae) are sprayed with an active compound preparation of the desired concentration.

After 5-6 days, the efficacy in % is determined. 100% means that all the aphids have been killed; 0% means that no aphids have been killed.

In this test, for example, the following compounds from the preparation examples show an efficacy of 100% at an application rate of 500 g/ha: 1, 6, 11, 13, 18, 22, 23, 25, 26, 27, 28, 29, 36, 42, 53, 54, 56, 57, 66, 75, 79, 84, 86, 87, 90, 92, 94, 96, 97, 100, 102, 107, 108, 109, 111, 115, 116, 117, 118, 119, 120, 138, 140, 142, 147, 151, 153, 156, 157, 158, 164, 169, 173, 174, 181, 183, 185, 186, 193, 200, 204, 206, 211, 212, 218, 219, 220, 225, 226, 227, 228, 229, 230, 235, 238, 269, 271, 272, 273, 274, 277, 278, 283, 284, 290, 294, 295, 298, 301, 303, 304.315, 318, 319, 320, 321, 323, 324, 342, 343, 349, 350, 354, 358, 363, 364, 365, 369, 372, 373, 374, 381, 383, 385, 387, 390, 393, 394, 395, 397, 399, 401, 403, 419, 420, 421, 423, 425, 426, 427, 428, 430, 432, 434, 435, 437, 438, 440, 445, 448, 449, 450, 460, 461, 463, 464, 465, 467, 491, 492, 493, 499, 500, 501, 510, 513, 514, 528, 529, 540, 549, 553, 559, 560, 562, 563, 564, 565, 566, 567, 568, 569, 570, 571, 572, 574, 576, 578, 579, 581, 582, 583, 584, 585, 586, 589, 591, 592, 593, 596, 597, 599, 600, 605, 608, 610

In this test, for example, the following compounds from the preparation examples show an efficacy of 90% at an application rate of 500 g/ha: 2, 5, 7, 8, 9, 10, 12, 14, 15, 16, 17, 19, 24, 30, 32, 33, 34, 37, 38, 39, 41, 43, 44, 45, 46, 47, 48, 51, 52, 55, 58, 59, 62, 64, 65, 67, 69, 70, 71, 72, 78, 80, 81, 82, 83, 89, 91, 93, 95, 98, 99, 101, 106, 110, 112, 113, 114, 121, 122, 123, 124, 125, 126, 127, 139, 143, 145, 148, 149, 150, 152, 154, 155, 159, 160, 162, 165, 166, 167, 168, 170, 171, 172, 175, 176, 178, 179, 182, 184, 188, 197, 207, 217, 224, 231, 257, 266, 268, 270, 275, 279, 280, 281, 282, 285, 289, 296, 297, 300, 310, 312, 313, 316, 317, 335, 336, 337, 338, 339, 341, 346, 352, 356, 357, 367, 370, 371, 388, 389, 391, 398, 400, 413, 422, 424, 429, 431, 433, 436, 439, 441, 442, 443, 444, 446, 447, 453, 454, 455, 458, 459, 462, 466, 469, 483, 488, 490, 497, 503, 512, 515, 517, 519, 520, 524, 526, 539, 542, 547, 548, 550, 552, 554, 555, 556, 557, 558, 561, 573, 575, 587, 588, 594, 595, 598, 602, 603, 604, 607, 609, 611

In this test, for example, the following compounds from the preparation examples show an efficacy of 90% at an application rate of 100 g/ha: 351, 378, 613

Myzus persicae—Spray Test

Solvent: 14 parts by weight of dimethylformamide

Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Bell pepper plants (Capsicum annuum) severely infested with the green peach aphid (Myzus persicae) are treated by spraying with the active compound preparation in the desired concentration.

After 6 days, the kill in % is determined. 100% means that all of the aphids have been killed; 0% means that none of the aphids have been killed.

In this test, for example, the following compounds from the preparation examples show an efficacy of 100% at an application rate of 100 ppm: 68, 415, 538

In this test, for example, the following compound from the preparation examples shows an efficacy of 99% at an application rate of 100 ppm: 478

In this test, for example, the following compound from the preparation examples shows an efficacy of 97% at an application rate of 100 ppm: 616

In this test, for example, the following compound from the preparation examples shows an efficacy of 95% at an application rate of 100 ppm: 518

In this test, for example, the following compounds from the preparation examples show an efficacy of 90% at an application rate of 100 ppm: 146, 237, 347, 533

In this test, for example, the following compound from the preparation examples shows an efficacy of 85% at an application rate of 100 ppm: 60

In this test, for example, the following compound from the preparation examples shows an efficacy of 80% at an application rate of 100 ppm: 536

In this test, for example, the following compound from the preparation examples shows an efficacy of 80% at an application rate of 20 ppm: 195

Phaedon cochleariae—Spray Test

| | |
|---|---|
| Solvent: | 78.0 parts by weight of acetone |
| | 1.5 parts by weight of dimethylformamide |
| Emulsifier: | alkylaryl polyglycol ether |

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) are sprayed with an active compound preparation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples show an efficacy of 100% at an application rate of 500 g/ha: 2, 18, 23, 27, 36, 37, 42, 56, 58, 100, 101, 102, 104, 105, 106, 107, 108, 120, 143, 152, 153, 164, 165, 172, 186, 188, 195, 196, 205, 216, 218, 221, 222, 231, 279, 280, 282, 285, 286, 289, 290, 294, 300, 304, 307, 308, 309, 313, 316, 318, 319, 324, 334, 336, 337, 338, 339, 340, 341, 361, 363, 364, 369, 370, 373, 383, 385, 387, 394, 397, 398, 399, 401, 405, 423, 424, 425, 428, 429, 430, 434, 436, 439, 445, 446, 448, 449, 450, 461, 464, 465, 466, 467, 469, 479, 502, 510, 517, 519, 540, 550, 556, 558, 561, 576, 577, 579, 581, 583, 584, 586, 587, 597, 599, 603, 610, 611

In this test, for example, the following compounds from the preparation examples show an efficacy of 83% at an application rate of 500 g/ha: 171, 174, 176, 217, 227, 269, 303, 311, 413, 420, 460, 548, 552, 568, 572, 575, 582, 602

Tetranychus urticae—Spray Test, OP-Resistant

| | |
|---|---|
| Solvent: | 78.0 parts by weight of acetone |
| | 1.5 parts by weight of dimethylformamide |
| Emulsifier: | alkylaryl polyglycol ether |

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Discs of bean leaves (Phaseolus vulgaris) infested with all stages of the greenhouse red spider mite (Tetranychus urticae) are sprayed with an active compound preparation of the desired concentration.

After 6 days, the efficacy in % is determined. 100% means that all the spider mites have been killed; 0% means that no spider mites have been killed.

In this test, for example, the following compounds from the preparation examples show an efficacy of 100% at an application rate of 500 g/ha: 74, 217, 293

In this test, for example, the following compounds from the preparation examples show an efficacy of 90% at an application rate of 500 g/ha: 46, 70, 78, 82, 87, 101, 104, 173, 195, 212, 291, 315, 318, 351, 406, 407, 495, 568, 573, 582

Aphis gossypii—Spray Test

Solvent: 14 parts by weight of dimethylformamide

Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Cotton plants (Gossypium hirsutum) which are heavily infested by the cotton aphid (Aphis gossypii) are sprayed with an active compound preparation of the desired concentration.

After 6 days, the kill in % is determined. 100% means that all the aphids have been killed; 0% means that no aphids have been killed.

In this test, for example, the following compound from the preparation examples shows an efficacy of 100% at an application rate of 100 ppm: 194

In this test, for example, the following compound from the preparation examples shows an efficacy of 98% at an application rate of 100 ppm: 103

In this test, for example, the following compound from the preparation examples shows an efficacy of 95% at an application rate of 100 ppm: 502

### Diabrotica Balteata—Spray Test

| | |
|---|---|
| Solvent: | 78 parts by weight of acetone |
| | 1.5 parts by weight of dimethylformamide |
| Emulsifier: | alkylaryl polyglycol ether |

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Pre-swollen wheat grains (Triticum aestivum) are incubated in a multiwell plate filled with agar and a little water for one day (5 seed grains per cavity). The germinated wheat grains are sprayed with an active compound formulation of the desired concentration. Subsequently, each cavity is infected with 10-20 beetle larvae of Diabrotica balteata.

After 7 days, the efficacy in % is determined. 100% means that all maize plants have grown as in the untreated, uninfected control; 0% means that no maize plant has grown.

In this test, for example, the following compounds from the preparation examples show an efficacy of 100% at an application rate of 160 μg/well: 102, 218, 279, 336, 337, 339, 341, 364, 369, 394, 429, 518, 519, 558

In this test, for example, the following compounds from the preparation examples show an efficacy of 80% at an application rate of 160 μg/well: 280, 282, 361, 398, 399, 401, 405, 423, 424, 446, 450, 510

In this test, for example, the following compounds from the preparation examples show an efficacy of 80% at an application rate of 80 μg/well: 363, 397

Myzus persicae—Oral Test

Solvent: 100 parts by weight of acetone

To produce a suitable preparation of active compound, 1 part by weight of active compound is dissolved using the specified parts by weight of solvent and made up with water until the desired concentration is attained.

50 μl of the active compound preparation are transferred into microtitre plates and made up to a final volume of 200 μl with 150 μl of IPL41 insect medium (33%+15% sugar). Subsequently, the plates are sealed with parafilm, which a mixed population of green peach aphids (Myzus persicae) within a second microtitre plate is able to puncture and imbibe the solution through it.

After 5 days, the efficacy in % is determined. 100% means that all the aphids have been killed; 0% means that no aphids have been killed.

In this test, for example, the following compounds from the preparation examples show an efficacy of 100% at an application rate of 20 ppm: 1, 11, 13, 14, 15, 17, 18, 19, 21, 22, 23, 25, 26, 27, 28, 29, 30, 32, 33, 36, 37, 38, 40, 49, 52, 53, 54, 55, 56, 57, 58, 59, 60, 61, 62, 63, 66, 67, 68, 69, 70, 71, 91, 92, 93, 94, 95, 96, 97, 98, 99, 100, 101, 102, 103, 104, 106, 107, 108, 109, 110, 111, 112, 113, 114, 116.118, 119, 120, 124, 125, 132, 134, 140, 147, 148, 152, 153, 155, 156, 157, 158, 159, 162, 163, 169, 170, 171, 172, 173, 174, 178, 179, 180, 182, 183, 184, 185, 186, 187, 188, 192, 194, 195, 196, 198, 199, 201, 202, 205, 211, 212, 213, 214, 216, 216, 217, 218, 219, 220, 221, 225, 226, 233, 235, 237, 238, 239, 245, 247, 248, 257, 265, 266, 268, 269, 270, 271, 272, 273, 274, 275, 276, 277, 278, 279, 280, 281, 282, 283, 284, 285, 286, 289, 290, 294, 295, 297, 298, 300, 302, 303, 304, 306, 311, 312, 313, 314, 315, 316, 317, 318, 319, 320, 323, 324, 325, 331, 336, 337, 338, 339, 341, 342, 348, 352, 354, 358, 363, 364, 367, 369, 370, 371, 372, 373, 378, 381, 383, 385, 386, 387, 388, 389, 390, 393, 394, 397, 398, 399, 401, 403, 405, 411, 414, 415, 419, 420, 421, 422, 423, 424, 425, 426, 427, 428, 429, 430, 431, 432, 433, 434, 435, 436, 437, 439, 440, 443, 444, 446, 447, 448, 449, 452, 453, 454, 456, 459, 461, 463, 465, 466, 467, 469, 470, 478, 483, 490, 491, 492, 497, 498, 499, 500, 501, 502, 503, 509, 510, 511, 512, 513, 514, 515, 517, 518, 519, 520, 524, 525, 528, 532, 533, 536, 538, 539, 540, 549, 553, 554, 555, 556, 557, 558, 559, 560, 561, 562, 563, 564, 565, 566, 567, 568, 569, 570, 571, 572, 573, 574, 575, 576, 578, 579, 583, 584, 585, 587, 588, 589, 590, 591, 592, 593, 594, 595, 596, 597, 598, 599, 600, 602, 603, 605, 607, 609, 610, 611, 612, 613, 615, 616, 617, 618, 619

In this test, for example, the following compounds from the preparation examples show an efficacy of 90% at an application rate of 20 ppm: 7, 12, 39, 41, 43, 44, 45, 51, 75, 77, 81, 115, 117, 126, 130, 131, 136, 137, 141, 143, 150, 165, 176, 181, 191, 206, 208, 209, 215, 236, 296, 299, 301, 305, 327, 330, 343, 345, 347, 350, 353, 368, 395, 412, 416, 418, 438, 445, 446, 450, 462, 464, 477, 496, 531, 547, 548, 601, 604, 620, 625

In this test, for example, the following compounds from the preparation examples show an efficacy of 100% at an application rate of 4 ppm: 581, 582, 586

1. A product comprising a compound of formula (I) 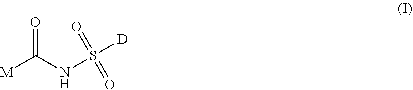
in which
M represents a radical selected from formulae (IIa-IIf): 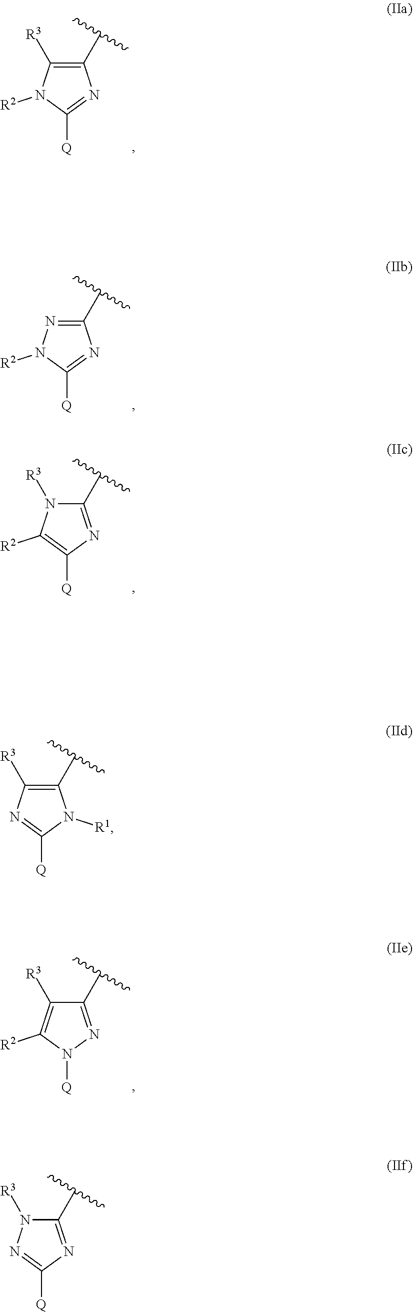
where
R¹, R², R³ each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
for controlling animal pests.

2. A product according to claim 1 where M represents a radical selected from formulae (IIa-IIf), where R¹, R², R³ each independently of one another represent H or a substituted or unsubstituted (C₁-C₄)-alkyl, (C₃-C₆)cycloalkyl, (C₂-C₄)-alkenyl, pyridyl or phenyl radical, where in cases (IIc) and (IIe) R² may additionally represent a halogen radical or an alkoxy radical and where in cases (IIa), (IId) and (IIe) R³ may additionally represent a halogen radical;
Q represents a substituted or unsubstituted phenyl, naphthyl or heteroaryl radical which may contain one to three heteroatoms from the group consisting of oxygen, sulphur, nitrogen; but, in case (IIe), does not represent 2-pyrimidinyl;
D represents a substituted or unsubstituted alkyl, heteroalkyl, cycloalkyl, heteroaryl, aryl or phenyl-(C₁-C₈)-alkyl radical or a substituted or unsubstituted nitrogen radical.

3. A product according to claim 1 where
M represents a radical selected from the formulae (IIa-IIf), where R¹, R², R³ are defined as in claim 1 and
Q represents a phenyl, naphthyl, pyridyl, pyrimidinyl, thiophene, benzothiophene, isoquinoline, benzodioxole or pyrazole radical which is unsubstituted or substituted by one or more radicals R⁴, but, in case (IIe), does not represent 2-pyrimidinyl,
where the substituent(s) R⁴ is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino) and/or aryl, aryloxy or hetaryl, optionally mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)alkenylaminocarbonyl, (C₃-C₈)-cycloalkylaminocarbonyl, (C₁-C₆)-alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino and (C₁-C₆)-alkylcarbonylamino and
D represents a radical, unsubstituted or substituted by one or more radicals R⁵, from the group consisting of (C₁-C₆)-alkyl, phenyl, phenyl-(C₁-C₂)-alkyl and heteroaryl which may contain one to three heteroatoms from the group consisting of oxygen, sulphur, nitrogen, or represents an NR⁶R⁷ radical,
where the substituent(s) R⁵ is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino) and (1-pyrazolyl)-(C₁-C₃)-alkyl and/or
aryl or hetaryl, mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)alkenylaminocarbonyl, (C₃-C₈)-cycloalkylaminocarbonyl, (C₁-C₆)-alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino and (C₁-C₆)-alkylcarbonylamino
and where R⁶ and R⁷ each independently of one another represent H, (C₁-C₆)-alkyl or a substituted or unsubstituted phenyl radical or R⁶ and R⁷ together may form an unsubstituted or substituted 4- to 8-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R⁵.

4. A product according to claim 1 where
M represents a radical selected from the formulae (IIa-IIf), where R¹, R², R³ are defined as in claim 1 and
Q represents a phenyl, naphthyl, pyridyl, pyrimidinyl, thiophene, benzothiophene, isoquinoline, benzodioxole or pyrazole radical which is unsubstituted or substituted by one or more radicals R⁴, but, in case (IIe), does not represent 2-pyrimidinyl,
where the substituent(s) R⁴ is/are each independently of one another selected from:
cyano, halogen, nitro, acetyl, hydroxy, carboxy, amino, tri-(C₁-C₆)-alkylsilyl, (C₃-C₈)cycloalkyl, (C₃-C₈)-cycloalkyl-(C₃-C₈)-cycloalkyl, (C₁-C₆)-alkyl-(C₃-C₈)-cycloalkyl, halo(C₃-C₈)-cycloalkyl, (C₁-C₆)-alkyl, (C₁-C₆)-haloalkyl, (C₁-C₆)-cyanoalkyl, (C₁-C₆)hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)haloalkoxy, (C₁-C₆)-cyanoalkoxy, (C₁-C₆)-alkoxycarbonyl-(C₁-C₆)-alkoxy, (C₁-C₆)-alkoxy(C₁-C₆)-alkoxy, (C₁-C₆)-alkylhydroxyimino, (C₁-C₆)-alkoxyimino, (C₁-C₆)-alkyl-(C₁-C₆)alkoxyimino, (C₁-C₆)-haloalkyl-(C₁-C₆)-alkoxyimino, (C₁-C₆)-alkylthio, (C₁-C₆)haloalkylthio, (C₁-C₆)-alkoxy-(C₁-C₆)-alkylthio, (C₁-C₆)-alkylthio-(C₁-C₆)-alkyl, (C₁-C₆)alkylsulphinyl, (C₁-C₆)-haloalkylsulphinyl, (C₁-C₆)-alkoxy-(C₁-C₆)-alkylsulphinyl, (C₁-C₆)alkylsulphinyl-(C₁-C₆)-alkyl, (C₁-C₆)-alkylsulphonyl, (C₁-C₆)-haloalkylsulphonyl, (C₁-C₆)alkoxy-(C₁-C₆)-alkylsulphonyl, (C₁-C₆)-alkylsulphonyl-(C₁-C₆)-alkyl, (C₁-C₆)alkylsulphonyloxy, (C₁-C₆)-alkylcarbonyl, (C₁-C₆)-alkylthiocarbonyl, (C₁-C₆)haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, (C₁-C₆)alkylaminothiocarbonyl, di-(C₁-C₆)-alkylaminocarbonyl, di-(C₁-C₆)alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkyl sulphonylamino, (C₁-C₆)-alkylamino, di(C₁-C₆)-alkylamino, aminosulphonyl, (C₁-C₆)-alkylaminosulphonyl, di-(C₁-C₆)alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino, (C₁-C₆)-alkylcarbonylamino, phenyl, halophenyl, phenoxy or halophenoxy and
D represents a radical, unsubstituted or substituted by one or more radicals R⁵, from the group consisting of (C₁-C₆)-alkyl, phenyl, phenyl-(C₁-C₂)-alkyl and heteroaryl which may contain one to three heteroatoms from the group consisting of oxygen, sulphur and nitrogen, or represents an NR⁶R⁷ radical,
where the substituent(s) R⁵ is/are each independently of one another selected from:
cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino or (1-pyrazolyl)-(C₁-C₃)-alkyl and
R⁶ and R⁷ each independently of one another represent H, a (C₁-C₆)-alkyl, (C₁-C₆)-haloalkyl, (C₁-C₆)-alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl, (C₁-C₆)-alkoxy(C₁-C₆)-alkyl
or
R⁶ and R⁷ together may form an unsubstituted or substituted 5- to 6-membered saturated or optionally fully or partially unsaturated ring which may be interrupted by 1 to 3 heteroatoms from the group consisting of oxygen, sulphur and nitrogen and which may be provided by one or more substitutions corresponding to one of the definitions of R⁵.

5. A product according to claim 1 where
M represents a radical selected from the formulae (IIa-IIf), where
in case (IIa) R², R³ each independently of one another represent H or a substituted or unsubstituted (C₁-C₄)-alkyl, (C₃-C₆)-cycloalkyl or phenyl radical, where R³ may additionally represent a halogen radical,
in case (IIb) R² represents H or a substituted or unsubstituted (C₁-C₄)-alkyl or (C₃-C₆)cycloalkyl radical,
in case (IIc) R², R³ each independently of one another represent H or a substituted or unsubstituted (C₁-C₄)-alkyl, (C₃-C₆)-cycloalkyl or phenyl radical, where R² may additionally represent a halogen or (C₁-C₄)-alkoxy radical,
in case (IId) R¹, R³ each independently of one another represent H or a substituted or unsubstituted (C₁-C₄)-alkyl or (C₃-C₆)-cycloalkyl radical, where R³ may additionally represent a halogen radical,
in case (IIe) R², R³ each independently of one another represent H, halogen or a substituted or unsubstituted (C₁-C₄)-alkyl, (C₃-C₆)-cycloalkyl, pyridyl or phenyl radical, and R² may additionally represent a (C₁-C₄)-alkoxy radical and
in case (IIf) R³ represents H or a substituted or unsubstituted (C₁-C₄)-alkyl or (C₃-C₆)cycloalkyl radical
and
D represents a (C₁-C₆)-alkyl, phenyl, naphth-2-yl, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R⁵ or represents an NR⁶R⁷ radical,
where the substituent(s) R⁵ is/are each independently of one another selected from:
cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino or (1-pyrazolyl)-(C₁-C₃)-alkyl and
R⁶ and R⁷ each independently of one another represent H, a (C₁-C₆)-alkyl, (C₁-C₆)-haloalkyl, (C₁-C₆)-alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl or (C₁-C₆)-alkoxy(C₁-C₆)-alkyl,
or form a ring from the group pyrrolidine, morpholine, piperidine.

6. A product according to claim 1 where
M represents a radical selected from the radicals of the formulae (IIa) to (IIf) where
in case (IIa) R² represents H, methyl or ethyl or optionally halogen-substituted phenyl and R³ represents H, methyl, ethyl, isopropyl or halogen,
in case (IIb) R² represents H, methyl or ethyl,
in case (IIc) R² represents H or methyl and R³ represents H, methyl or ethyl,
in case (IId) R¹ represents H or methyl and R³ represents H or halogen,
in case (IIe) R² represents H, methyl, methoxy, halogen-substituted phenyl or halogen-substituted pyridyl and R³ represents H,
in case (IIf) R³ represents H, methyl or ethyl.

7. A product according to claim 1 where
M is selected from one of the formulae (IIa) to (IIf) or (IVa) to (IVf), and
Q represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R⁴, but, in case (IVe), does not represent 2-pyrimidinyl,
where the substituent(s) R⁴ independently of one another represent(s):
cyano, halogen, nitro, acetyl, (C₃-C₆)-cycloalkyl, (C₃-C₆)-cycloalkyloxy, (C₃-C₆)cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)-hydroxyalkyl, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)haloalkylsulphinyl, (C₁-C₆)-alkylsulphonyl, (C₁-C₆)-haloalkylsulphonyl, (C₁-C₆)alkylsulphonyloxy, (C₁-C₆)-alkylcarbonyl, (C₁-C₆)-alkylamino, di-(C₁-C₆)-alkylamino, (C₁-C₆)-alkylcarbonylamino, phenyl, halophenyl, phenoxy or halophenoxy and
D represents a (C₁-C₆)-alkyl, phenyl, naphth-2-yl, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R⁵ or represents an NR⁶R⁷ radical,
where the substituent(s) R⁵ independently of one another represent(s):
cyano, halogen, nitro, acetyl, hydroxy, carboxy, amino, (C₃-C₆)-cycloalkyl, (C₃-C₆)cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)hydroxyalkyl, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)alkylsulphinyl, (C₁-C₆)-haloalkylsulphinyl, (C₁-C₆)-alkylsulphonyl, (C₁-C₆)haloalkylsulphonyl, (C₁-C₆)-alkylsulphonyloxy, (C₁-C₆)-alkylcarbonyl, (C₁-C₆)alkylamino, di-(C₁-C₆)-alkylamino, (C₁-C₆)-alkylcarbonylamino or (1-pyrazolyl)-(C₁-C₃)alkyl and
R⁶ and R⁷ each independently of one another represent H, (C₁-C₆)-alkyl, phenyl, alkoxyphenyl or halophenyl or form a ring from the group pyrrolidine, morpholine, piperidine.

8. A product according to claim 1 where
M represents a radical selected from the radicals of formulae (Va-Vz): 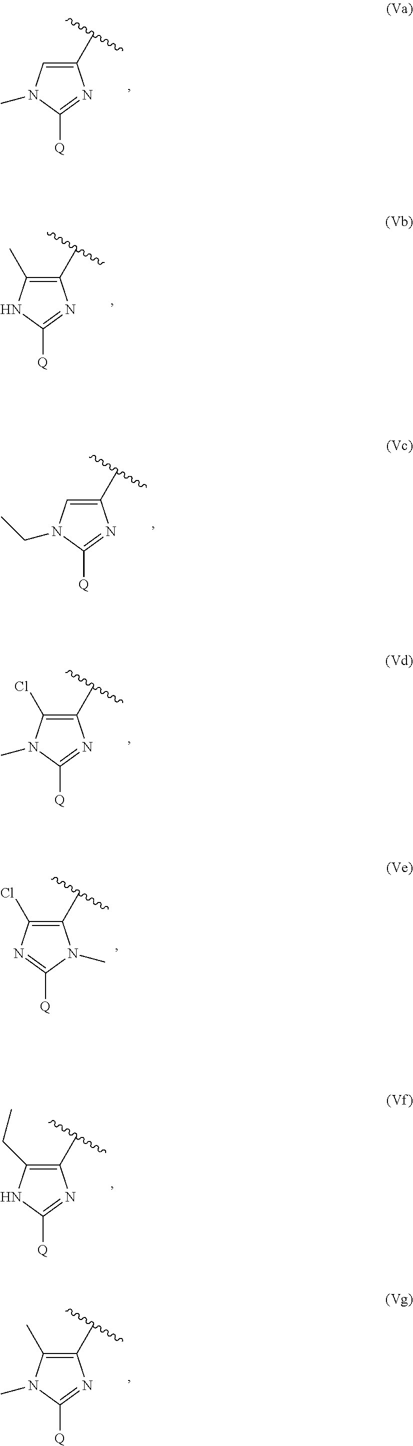 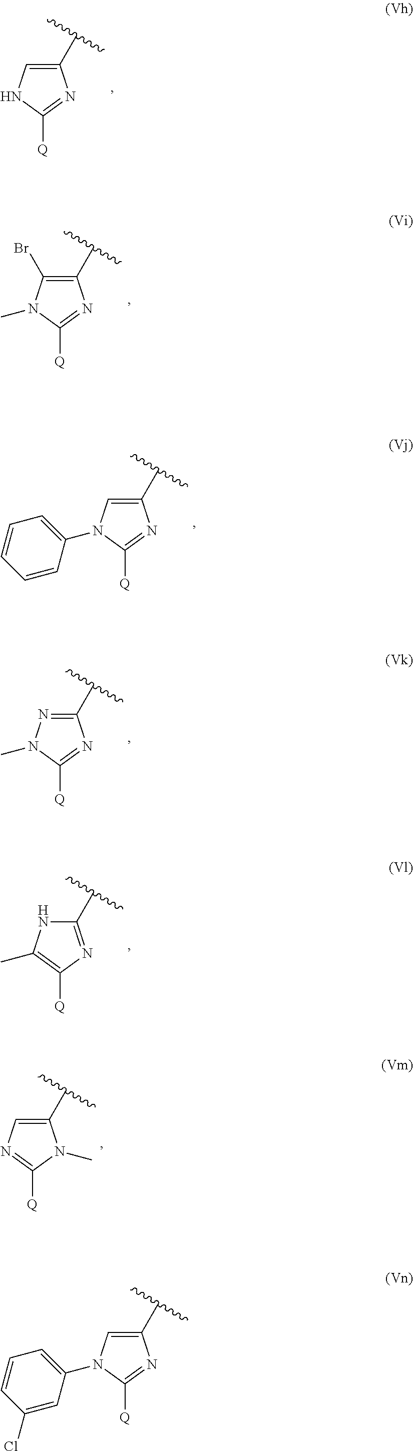 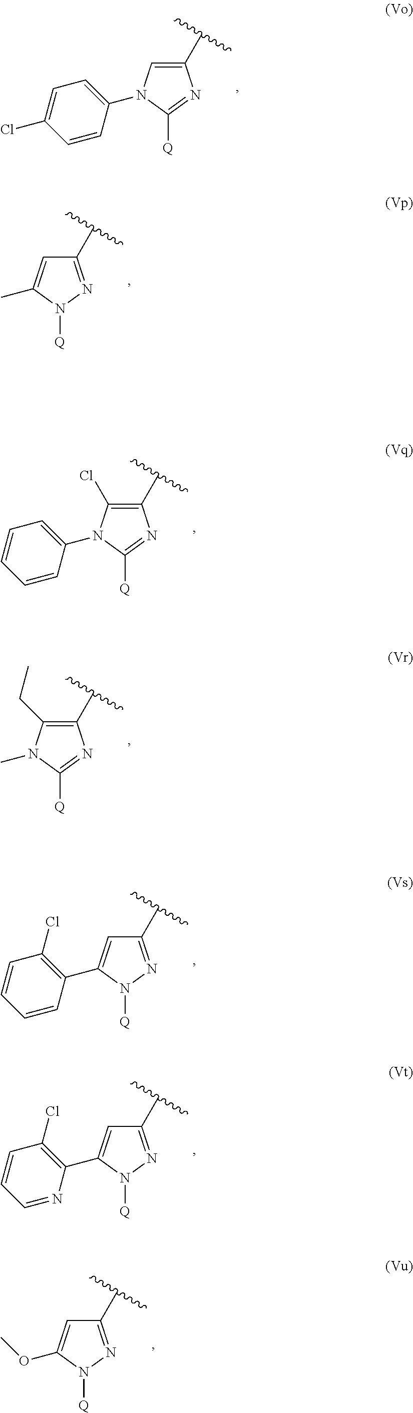 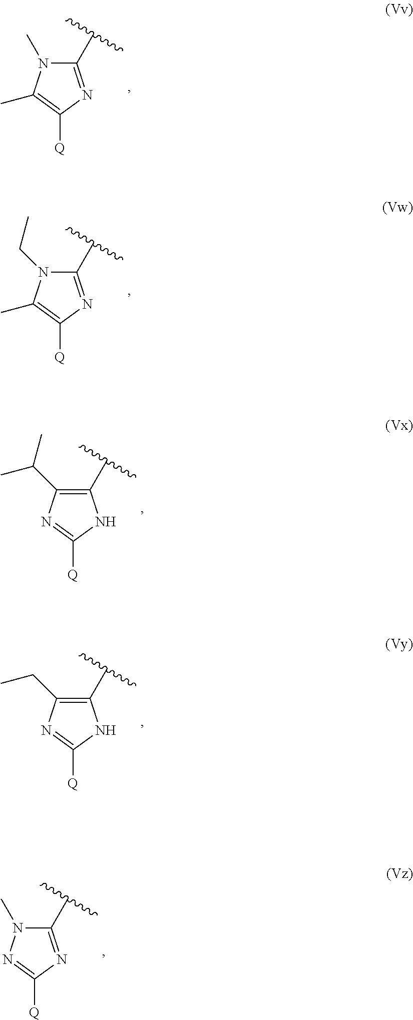
Q represents a radical selected from the radicals of formulae (VIa-VIz and VIa1-VIa30): 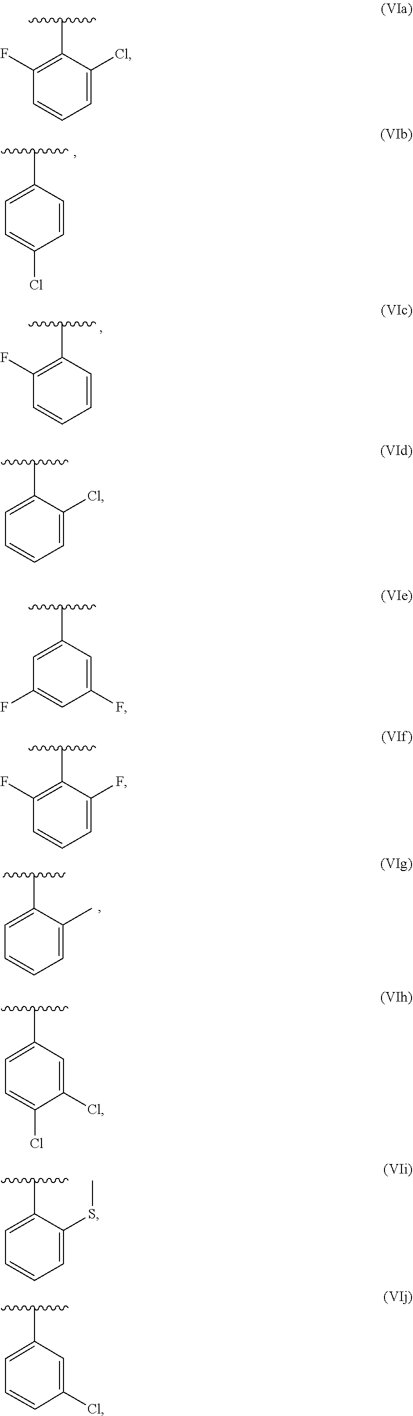 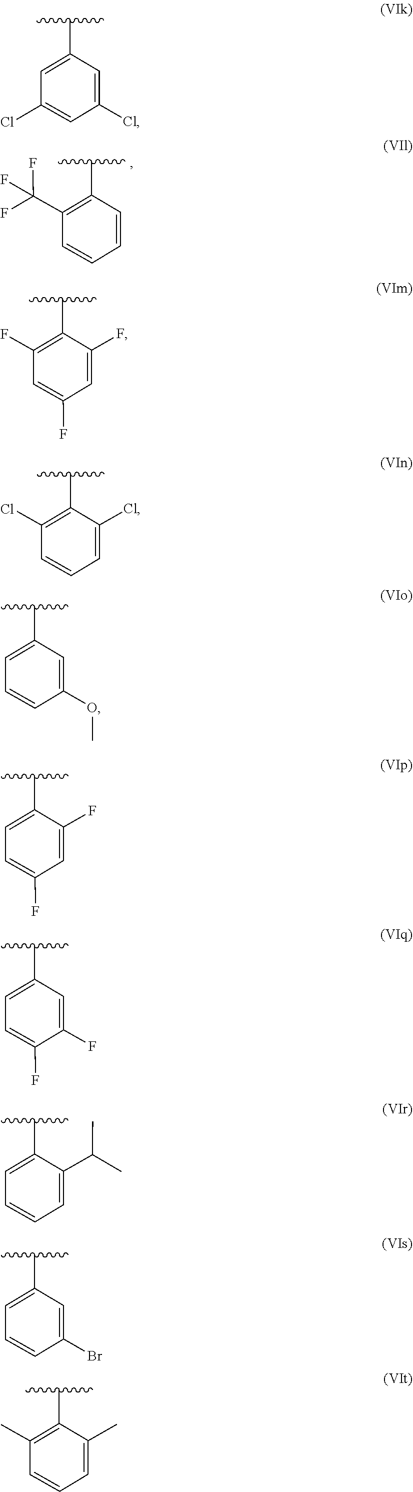 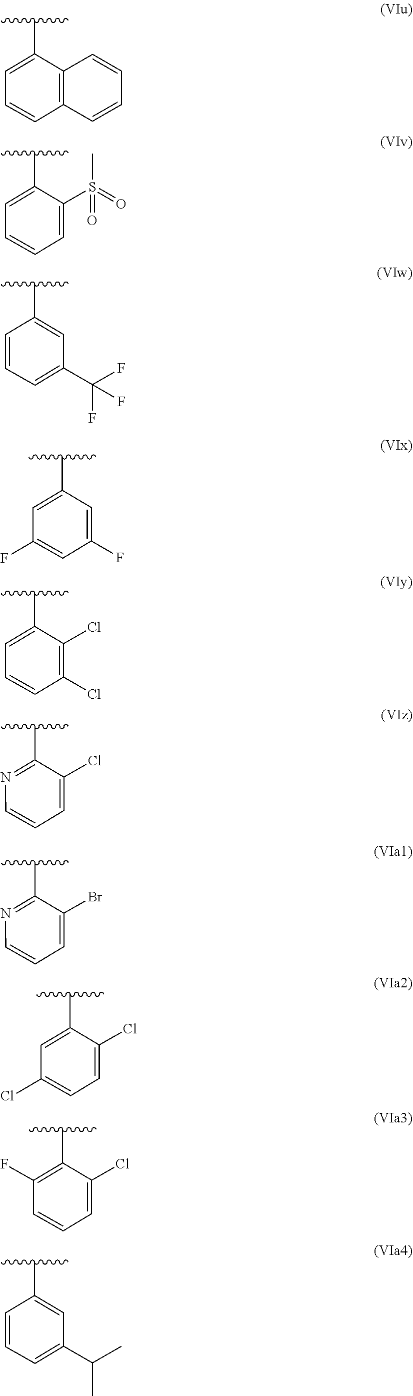 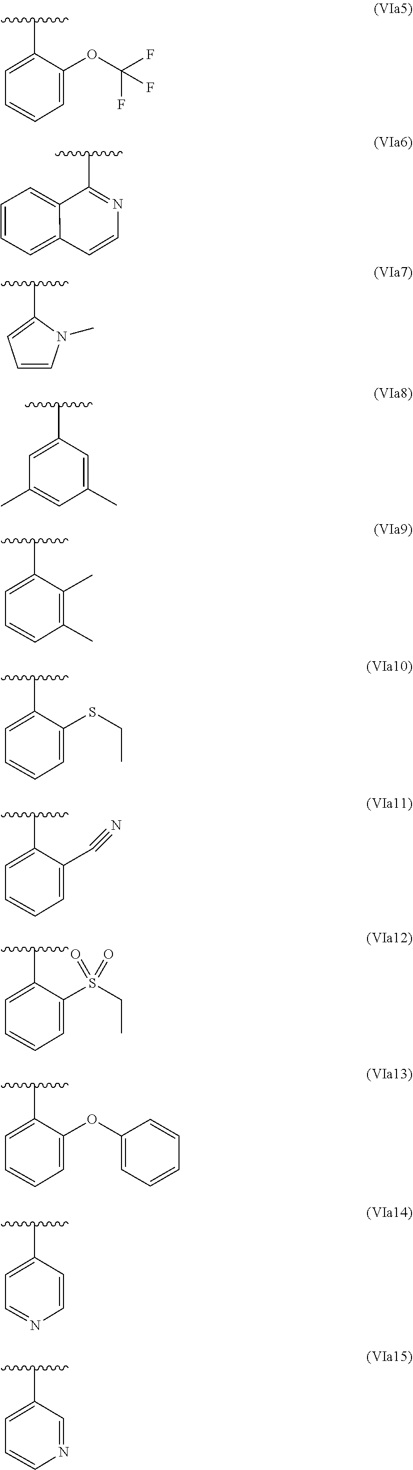 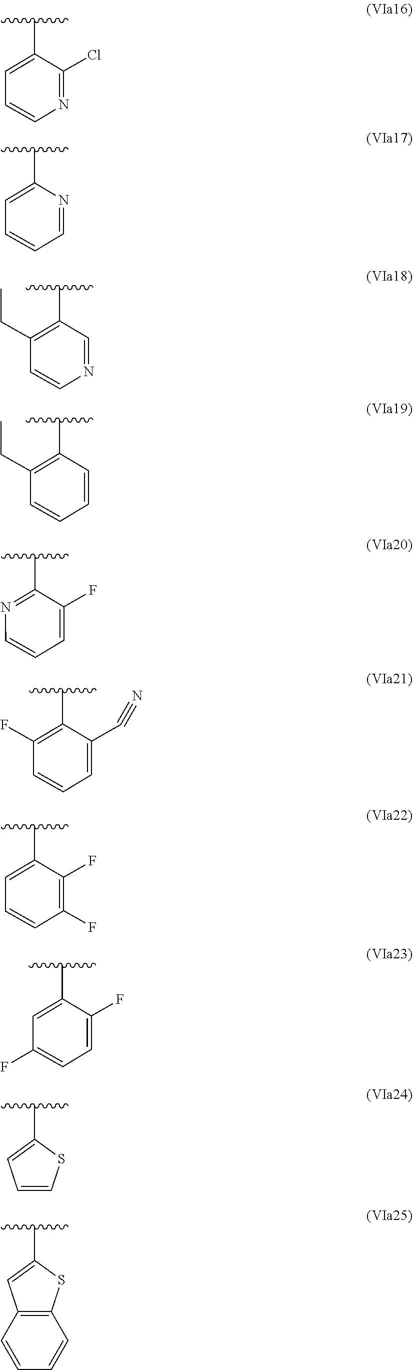 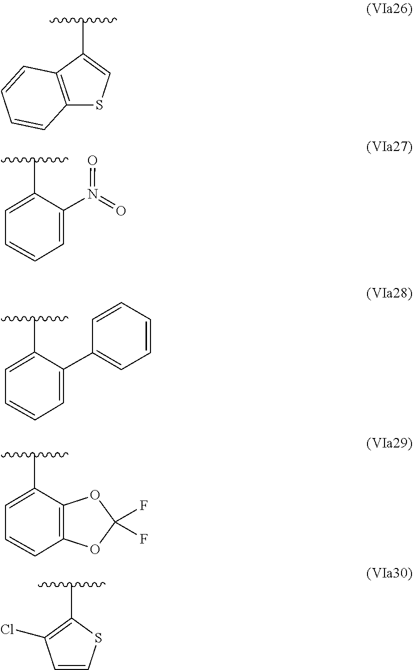
and
D represents a radical selected from the radicals of formulae (VII1-VII192)

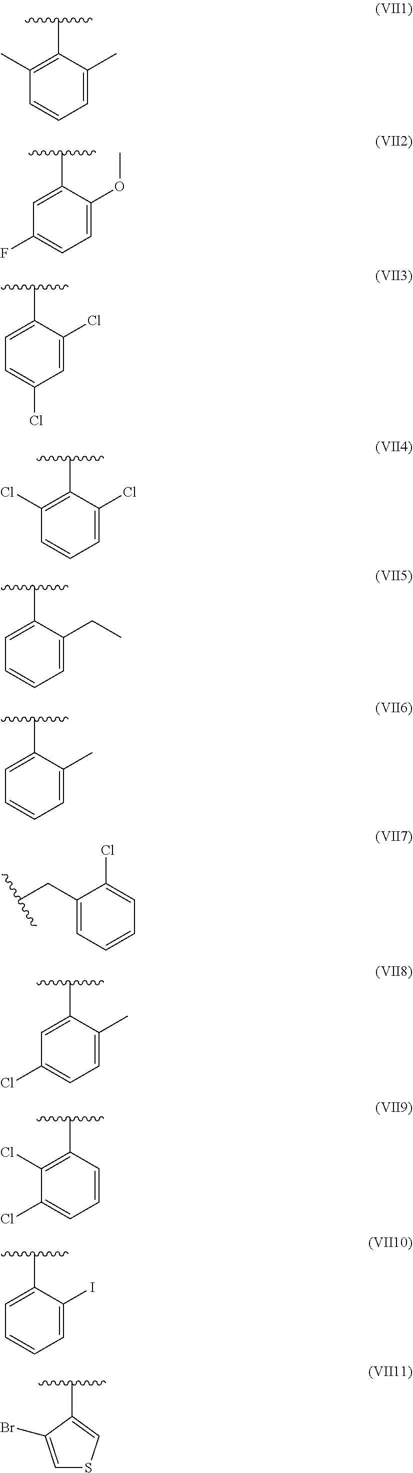

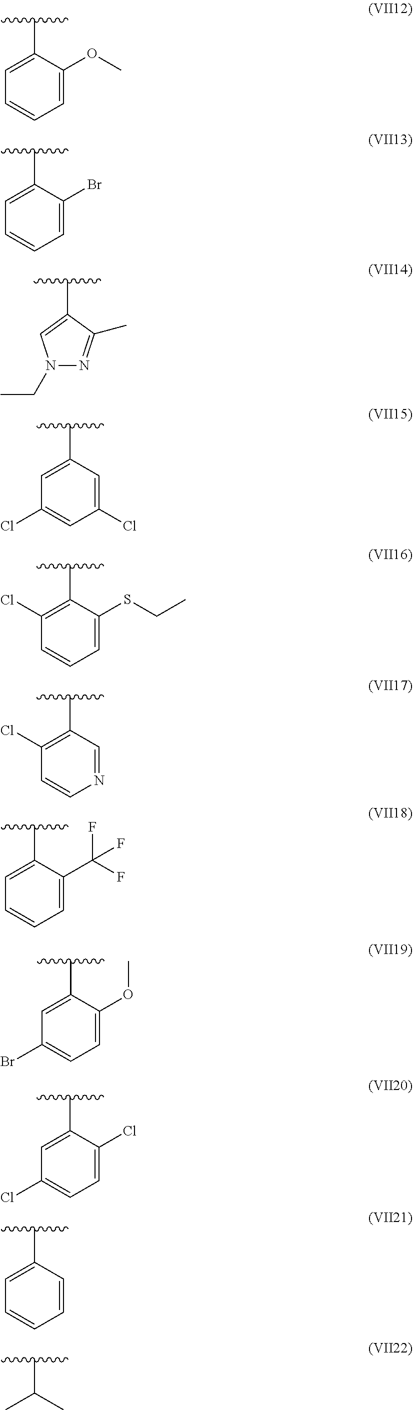

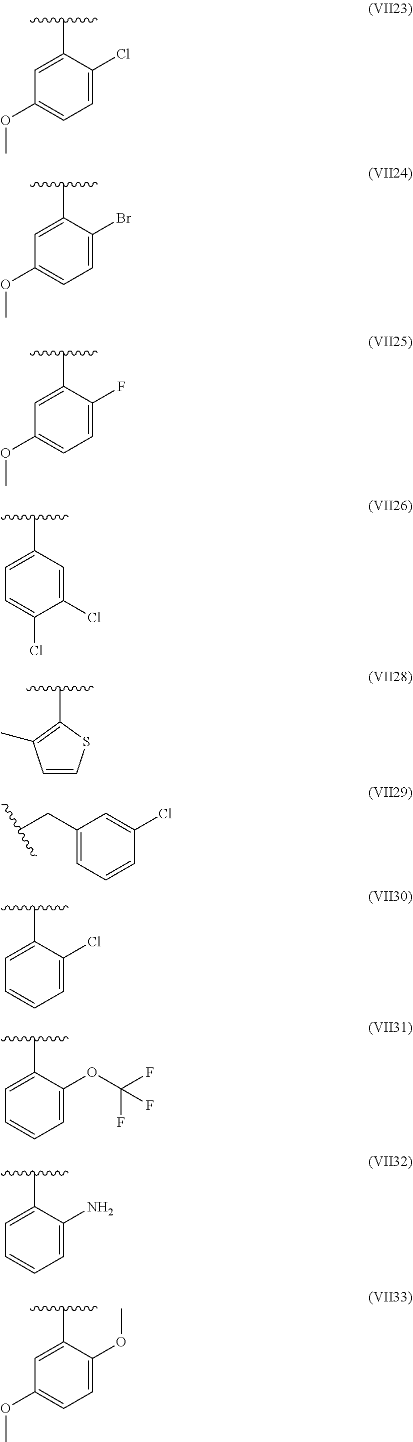

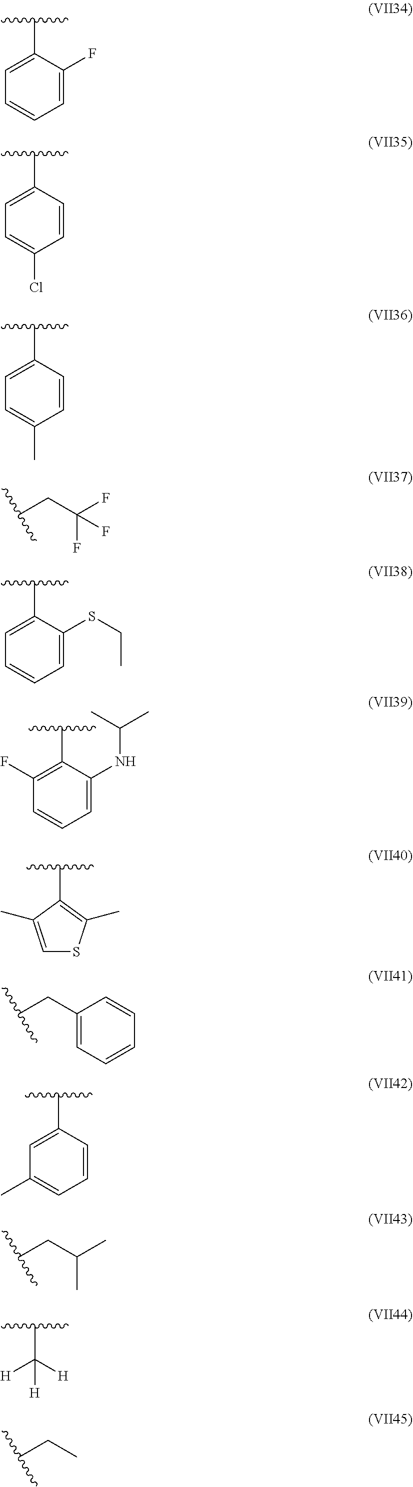

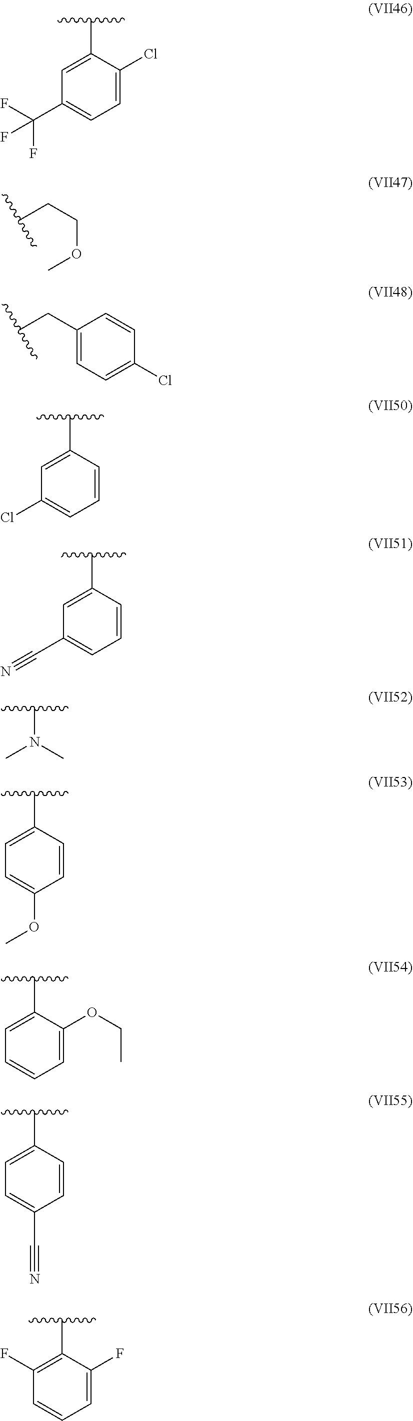

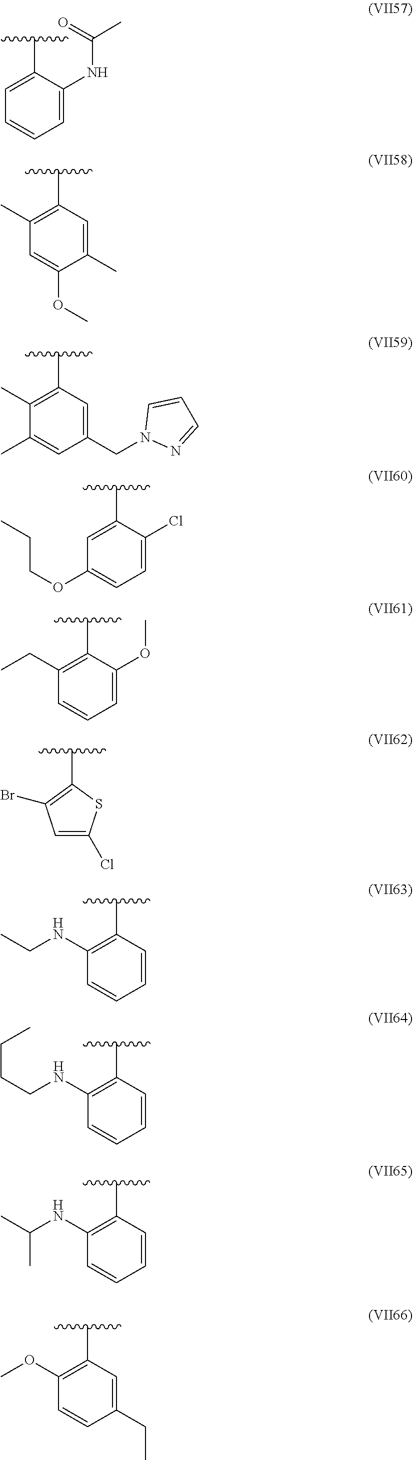

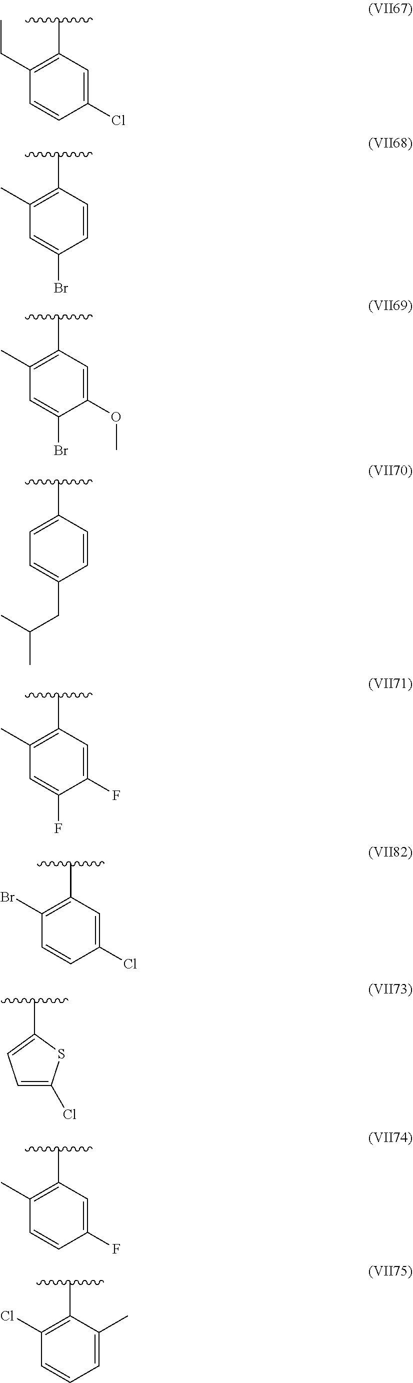

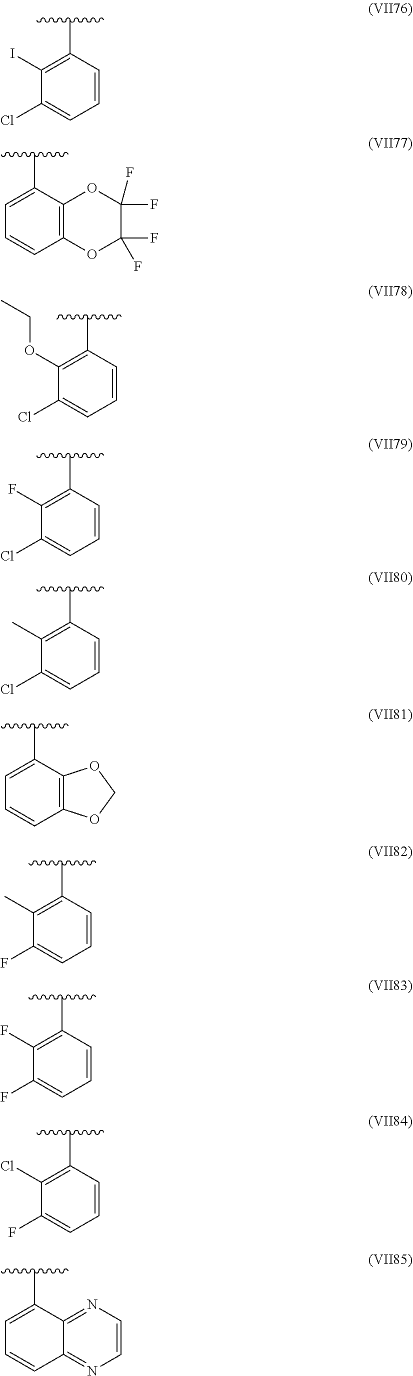

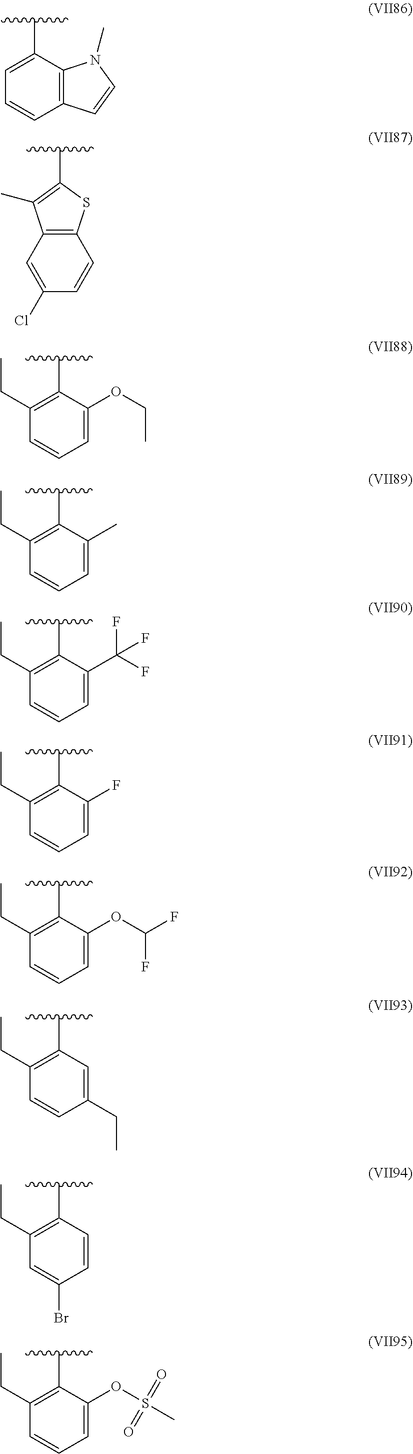

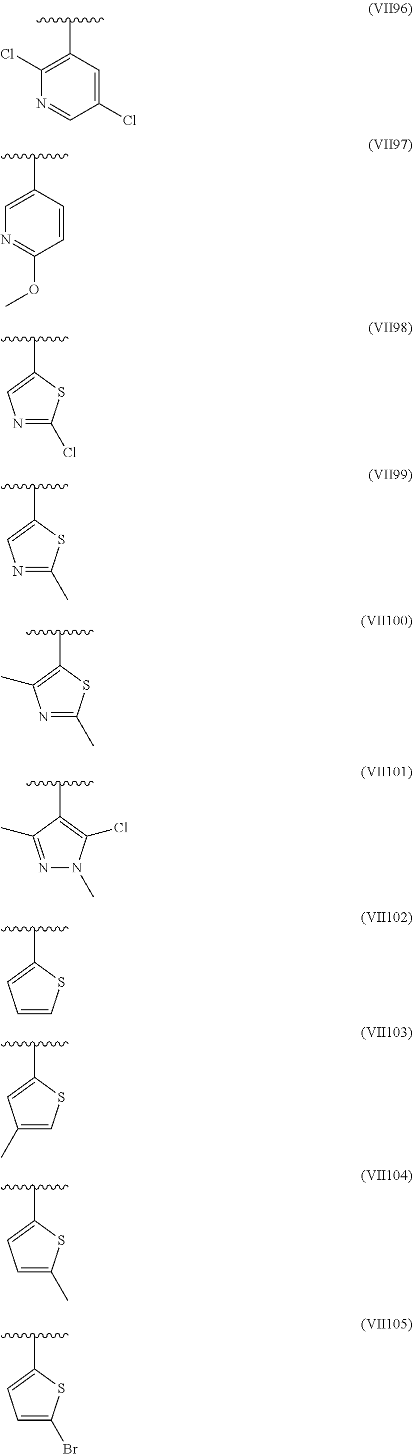

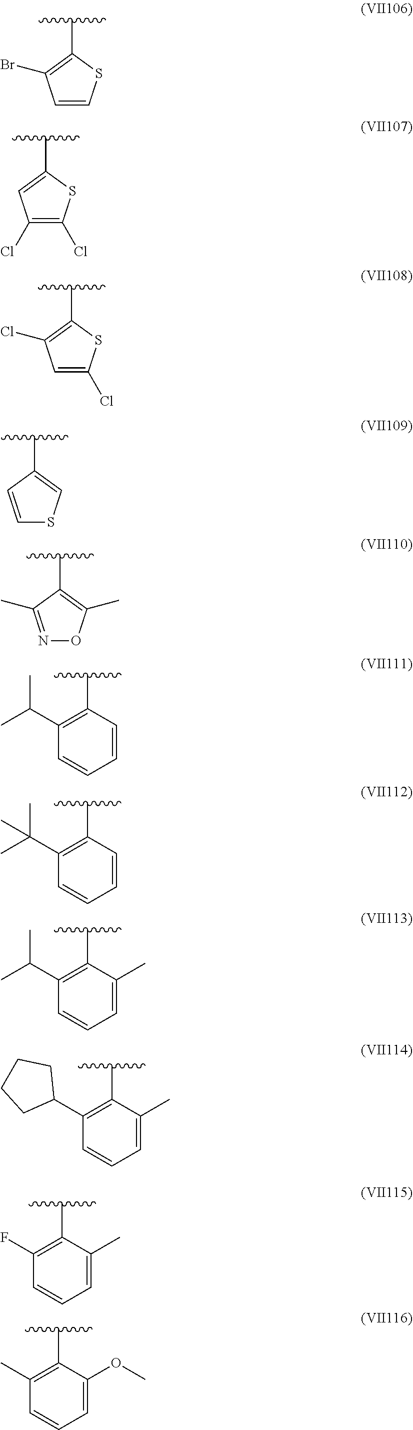

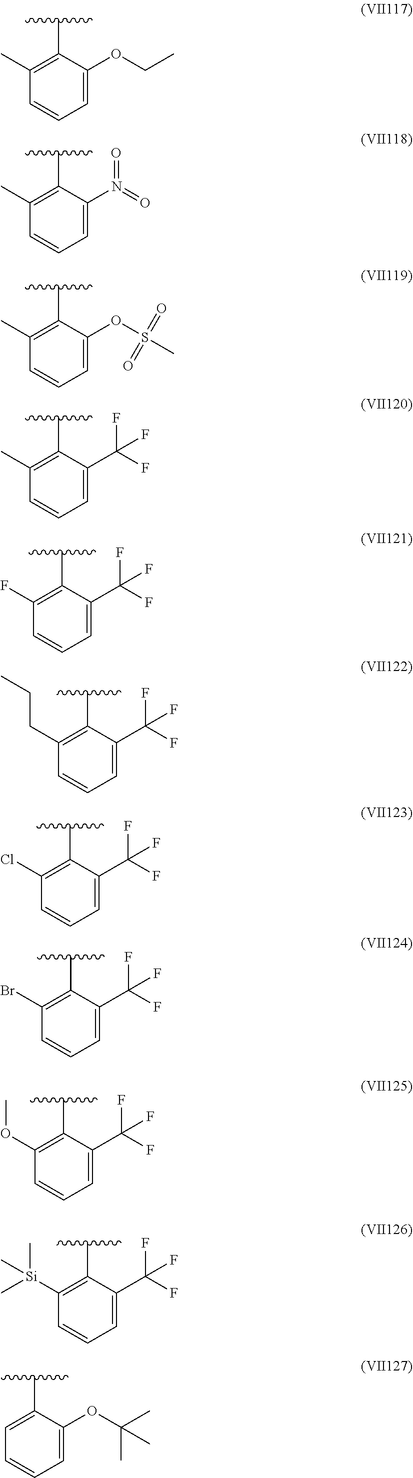

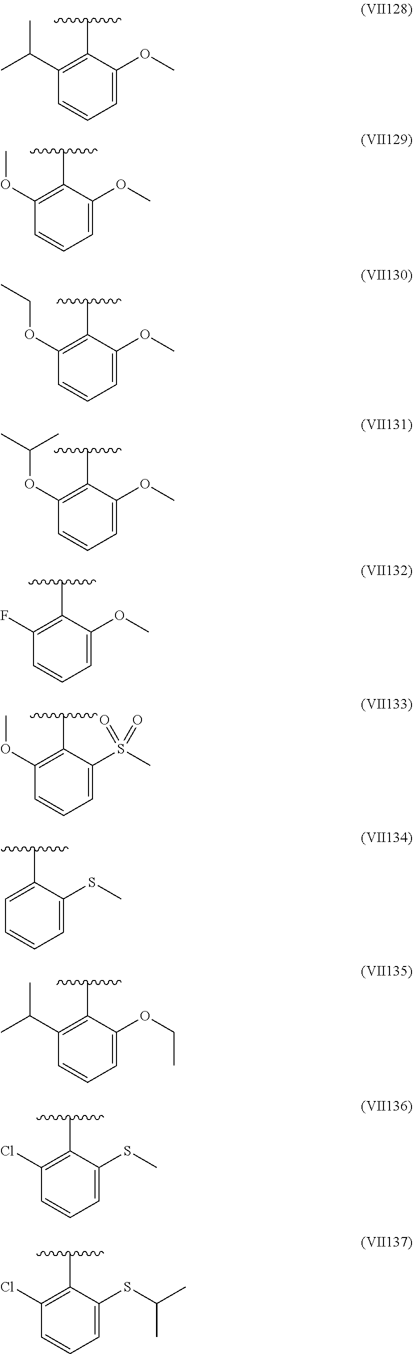

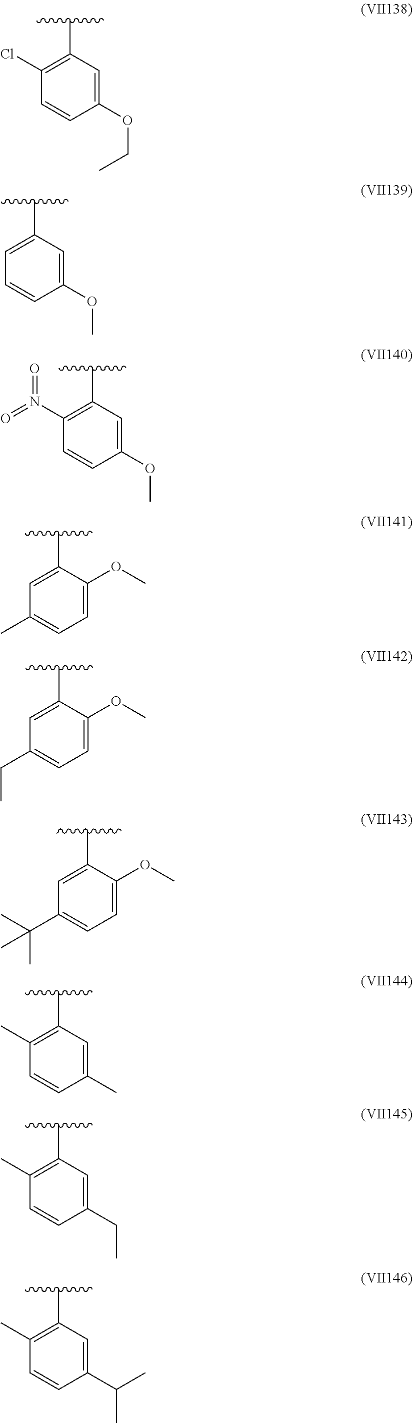

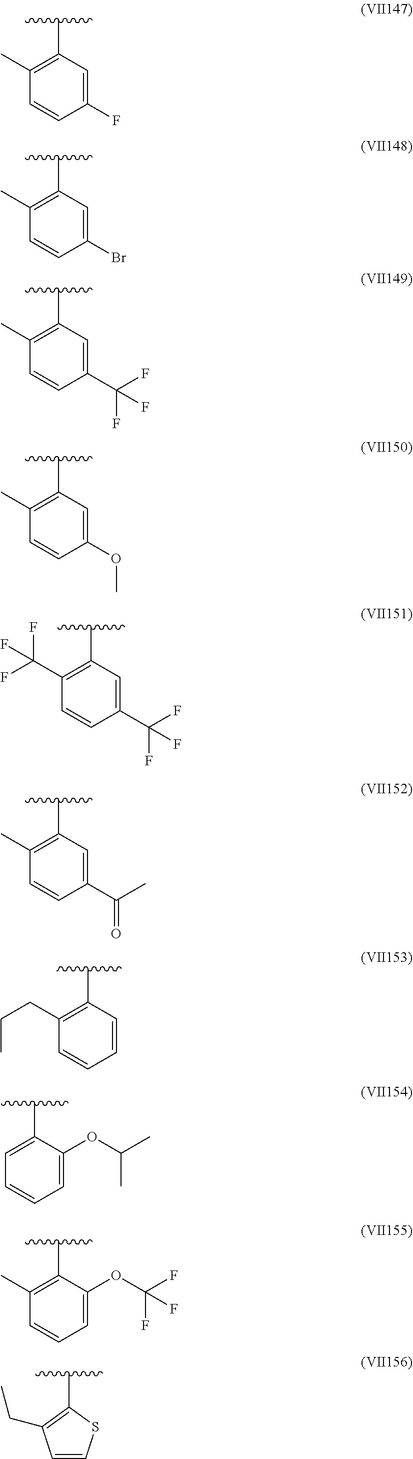

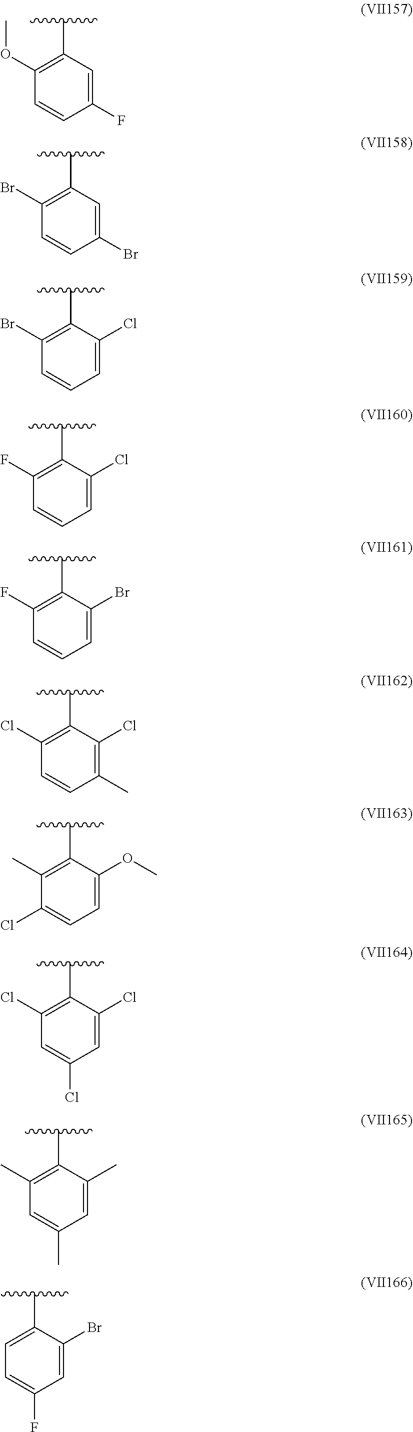

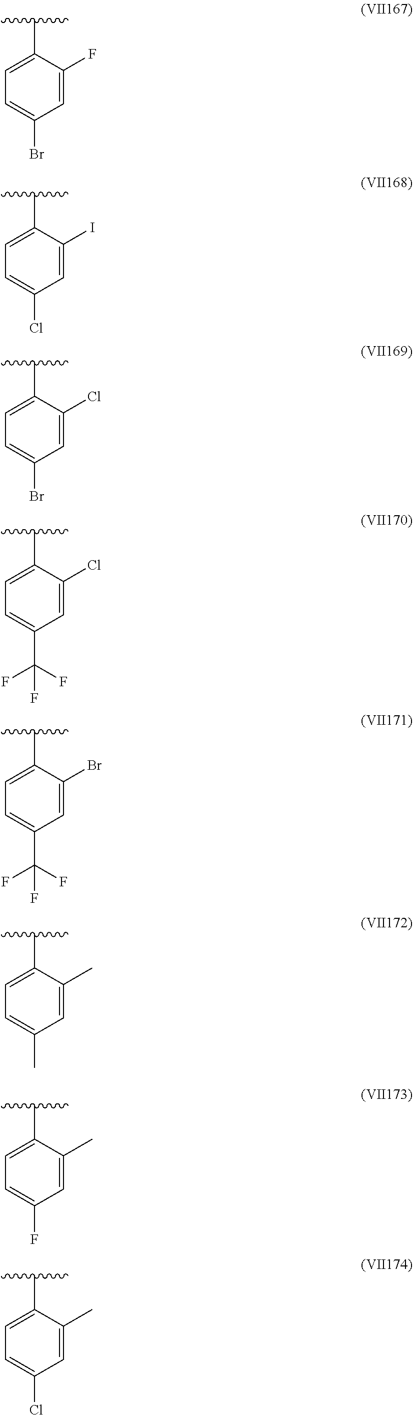

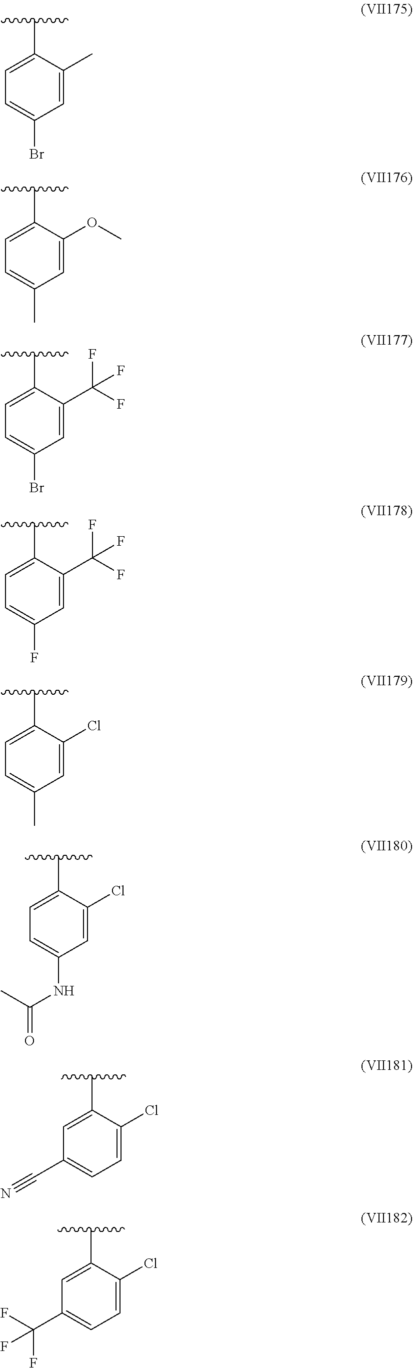

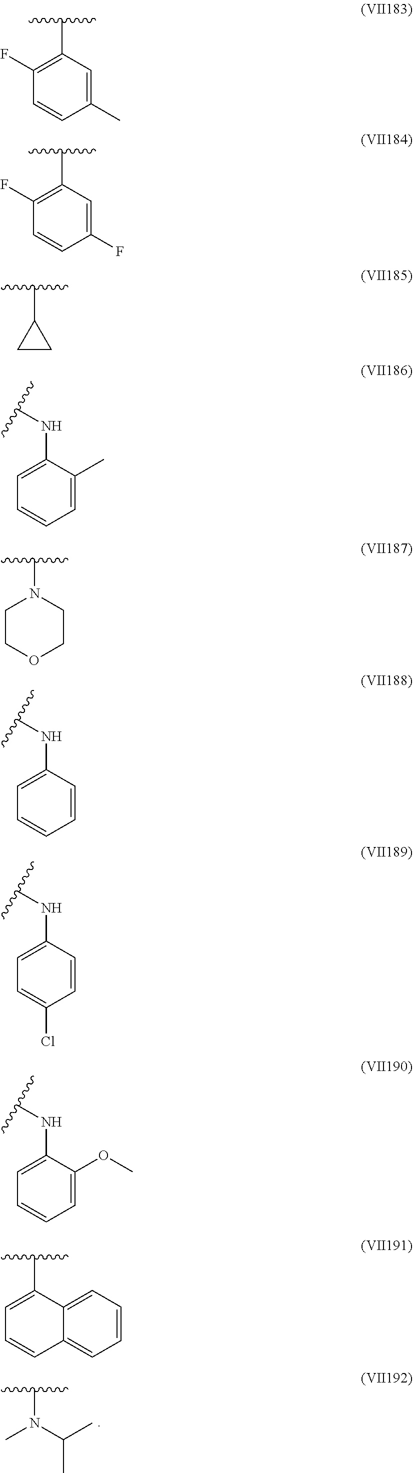

9. A product according to claim 1 for protecting the propagation material of plants.

10. Composition, comprising at least one compound of formula (I) formula (I) 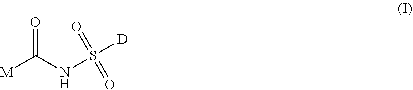
in which
M represents a radical selected from formulae (IIa-IIf): 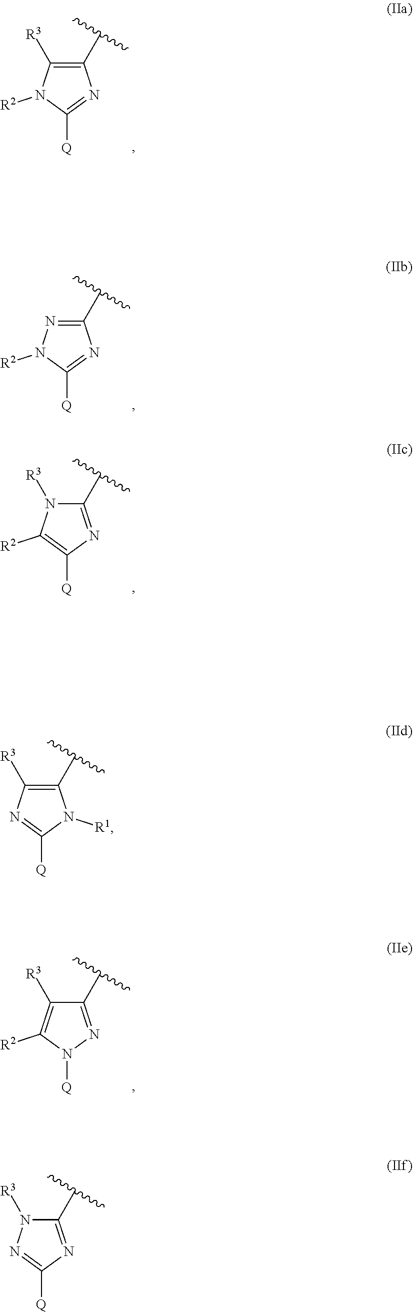
where
R¹, R², R³ each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
and one or more customary extenders and/or surfactants optionally for controlling animal pests.

11. Method for controlling animal pests, where at least one compound of formula (I)
formula (I) 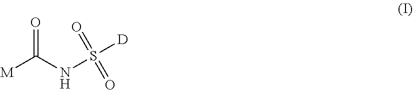
in which
M represents a radical selected from formulae (IIa-IIf): 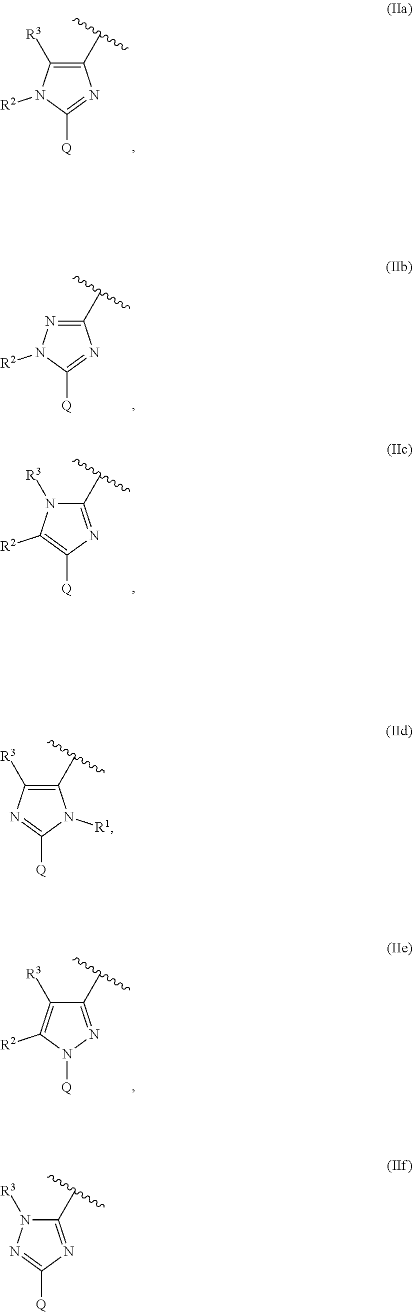
where
R¹, R², R³ each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
according to claim 1 is allowed to act on the animal pests and/or a habitat thereof.

12. Method according to claim 11, where the surgical, therapeutic and diagnostic treatment of a human or animal body is excluded.

13. Agrochemical formulation comprising at least one compound of formula (I)
formula (I) 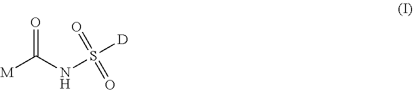
in which
M represents a radical selected from formulae (IIa-IIf): 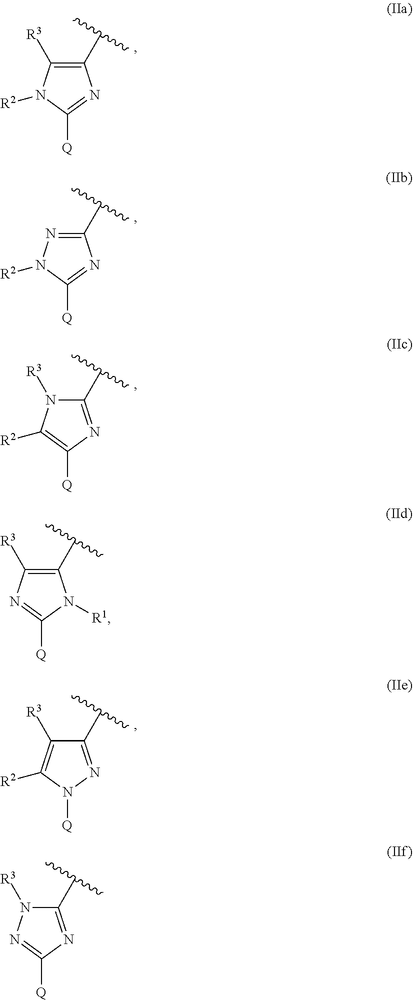
where
R¹, R², R³ each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, and in cases (IIa), (IId) and (IIe) R³ may additionally represent a halogen radical;
Q represents a substituted or unsubstituted aryl or heteroaryl radical, but, in case (IIe), does not represent 2-pyrimidinyl;
D represents a substituted or unsubstituted alkyl, heteroalkyl, optionally partially unsaturated cycloalkyl, cycloheteroalkyl, heteroaryl, aryl or phenylalkyl radical or a substituted or unsubstituted nitrogen radical,
in a biologically effective amount of from 0.00000001 to 98% by weight based on the weight of the agrochemical formulation, and one or more extenders and/or surfactants.

14. Agrochemical formulation according to claim 13, additionally comprising at least one further agrochemically active compound.

15. Compound of formula (VIII) 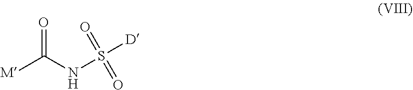
in which
M′ represents a radical of formula (II) selected from: 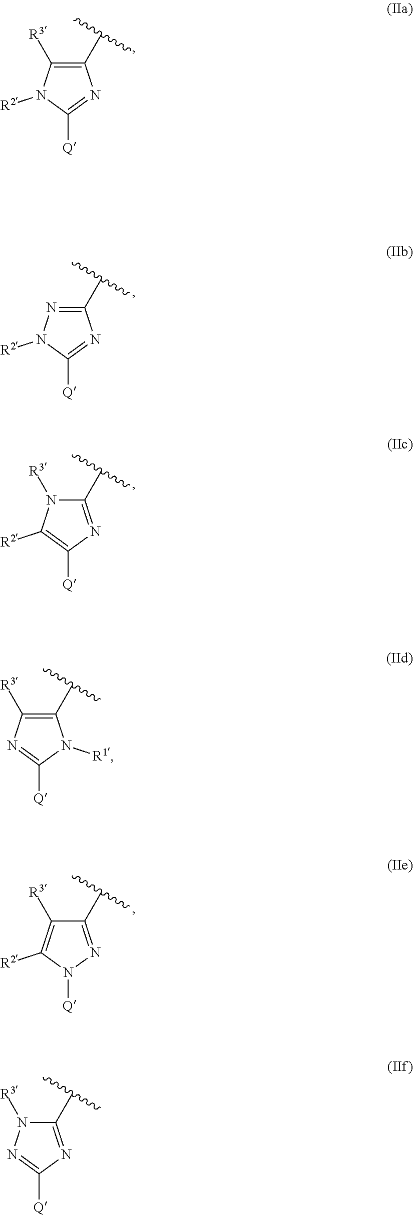
in which
R^{1′}, R^{2′}, R^{3′} each independently of one another represent H or a substituted or unsubstituted alkyl, cycloalkyl, alkenyl, cycloalkenyl, cycloheteroalkyl, aryl or heteroaryl radical, where in cases (IIc) and (IIe) R² may additionally represent a halogen radical or an alkoxy radical, in cases (IIa) and (IIb) R^{2′} may only represent H or a substituted or unsubstituted alkyl or cycloalkyl radical and in cases (IIa), (IId) and (IIe) R^{3′} may additionally represent a halogen radical,
Q′ represents a substituted or unsubstituted aryl or heteroaryl radical, but in cases (IIa), (IId)
in case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl, nor 3,5-bis-tert-butyl;
D′ represents a substituted or unsubstituted alkyl, heteroalkyl, optionally partially unsaturated cycloalkyl, cycloheteroalkyl, heteroaryl, aryl or phenylalkyl radical or in the case that Q′ carries at least one substituent in the 2-position represents a substituted or unsubstituted nitrogen radical.

16. Compound according to claim 15 in which
M′ represents a radical of one of formulae (IIa) to (IIf) and
in case (IIa) R^{2′}, R^{3′} each independently of one another represent H or a substituted or unsubstituted (C₁-C₄)-alkyl, (C₃-C₆)-cycloalkyl or phenyl radical, where R^{3′} may additionally represent a halogen radical;
in case (IIb) R^{2′} represents H or a substituted or unsubstituted (C₁-C₄)-alkyl or (C₃-C₆)cycloalkyl radical,
in case (IIc) R^{2′}, R^{3′} each independently of one another represent H or a substituted or unsubstituted (C₁-C₄)-alkyl, (C₃-C₆)-cycloalkyl or phenyl radical, where R^{2′} may additionally represent a halogen radical or a (C₁-C₄)-alkoxy radical,
in case (IId) R^{1′}, R^{3′} each independently of one another represent H or a substituted or unsubstituted (C₁-C₄)-alkyl or (C₃-C₆)-cycloalkyl radical and R^{3′} may additionally represent a halogen radical,
in case (IIe) R^{2′}, R^{3′} each independently represent H, halogen or a substituted or unsubstituted (C₁-C₄)-alkyl, (C₃-C₆)-cycloalkyl, pyridyl or phenyl radical, and R^{2′} may additionally represent a (C₁-C₄)-alkoxy radical and
in case (IIf) R^{3′} represents H or a substituted or unsubstituted (C₁-C₄)-alkyl or (C₃-C₆)cycloalkyl radical,
Q′ represents a substituted or unsubstituted phenyl, naphthyl or heteroaryl radical which may contain one to three heteroatoms from the group of oxygen, sulphur and nitrogen, but in cases (IIa), (IId) in case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl,
D′ represents a substituted or unsubstituted alkyl, heteroalkyl, cycloalkyl, heteroaryl, aryl or phenyl-(C₁-C₈)-alkyl radical or, in the case that Q′ carries at least one substituent in the 2-position, represents a substituted or unsubstituted nitrogen radical.

17. Compound according to claim 15 where
M′ represents a radical selected from the formulae (IIa-IIf), and
Q′ represents a phenyl, naphthyl, pyridyl, pyrimidinyl, thiophene, benzothiophene, isoquinoline, benzodioxole or pyrazole radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituent(s) R^{4′} is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino; and/or
aryl, aryloxy or hetaryl, optionally mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)alkenylaminocarbonyl, (C₃-C₈)-cycloalkylaminocarbonyl, (C₁-C₆)-alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino and (C₁-C₆)-alkylcarbonylamino and
D′ represents a radical, unsubstituted or substituted by one or more radicals R⁵, from the group consisting of (C₁-C₆)-alkyl, phenyl and heteroaryl which may contain one to three heteroatoms from the group consisting of oxygen, sulphur and nitrogen, or, in the case that Q′ carries at least one substituent in the 2-position, represents an NR^{6′}R^{7′} radical,
where the substituent(s) R^{5′} is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl, and/or
aryl or hetaryl, mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)alkenylaminocarbonyl, (C₃-C₈)-cycloalkylaminocarbonyl, (C₁-C₆)-alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino and (C₁-C₆)-alkylcarbonylamino,
and where R^{6′} and R^{7′} each independently of one another represent H, a (C₁-C₆)-alkyl, (C₁-C₆)-haloalkyl, (C₁-C₆)-alkoxy-(C₁-C₆)-alkyl radical or an unsubstituted phenyl radical or a phenyl radical substituted by halogen, (C₁-C₆)-alkyl, (C₁-C₆)-alkoxy, (C₁-C₆)-haloalkyl, (C₁-C₆)-alkoxy-(C₁-C₆)-alkyl, or
R^{6′} and R^{7′} together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R^{5′}.

18. Compound according to claim 15 where
M′ represents a radical selected from the formulae (IIa-IIf), and
Q′ represents a phenyl, naphthyl, pyridyl, pyrimidinyl, thiophene, benzothiophene, isoquinoline, benzodioxole or pyrazole radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl,
and the substituent(s) R^{4′} each independently of one another are selected from: cyano, halogen, nitro, acetyl, hydroxy, carboxy, amino, tri-(C₁-C₆)-alkylsilyl, (C₃-C₈)-cycloalkyl, (C₃-C₈)-cycloalkyloxy, (C₃-C₈)-cycloalkyl-(C₃-C₈)-cycloalkyl, (C₁-C₆)-alkyl-(C₃-C₈)cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)alkyl, (C₁-C₆)-alkoxy-(C₁-C₆)-alkyl, (C₂-C₆)-alkenyl, (C₂-C₆)-haloalkenyl, (C₂-C₆)cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulphinyl, (C₁-C₆)-haloalkylsulphinyl, (C₁-C₆)-alkoxy-(C₁-C₆)-alkylsulphinyl, (C₁-C₆)-alkylsulphinyl-(C₁-C₆)-alkyl, (C₁-C₆)-alkylsulphonyl, (C₁-C₆)-haloalkylsulphonyl, (C₁-C₆)-alkoxy-(C₁-C₆)-alkylsulphonyl, (C₁-C₆)-alkylsulphonyl-(C₁-C₆)-alkyl, (C₁-C₆)alkylsulphonyloxy, (C₁-C₆)-alkylcarbonyl, (C₁-C₆)-alkylthiocarbonyl, (C₁-C₆)haloalkylcarbonyl, (C₁-C₆)-alkylcarbonyloxy, (C₁-C₆)-alkoxycarbonyl, (C₁-C₆)haloalkoxycarbonyl, aminocarbonyl, (C₁-C₆)-alkylaminocarbonyl, (C₁-C₆)alkylaminothiocarbonyl, di-(C₁-C₆)-alkylaminocarbonyl, di-(C₁-C₆)alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkyl sulphonylamino, (C₁-C₆)-alkylamino, di(C₁-C₆)-alkylamino, aminosulphonyl, (C₁-C₆)-alkylaminosulphonyl, di-(C₁-C₆)alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino, (C₁-C₆)-alkylcarbonylamino, phenyl, halophenyl, phenoxy or halophenoxy and
D′ represents a phenyl, pyridine, pyrimidine, pyrazole, triazole, thiazole, oxazole, thiadiazole, oxadiazole, thiophene, pyrrole, furan, tetrahydrofuran, dioxane or (C₁-C₆)-alkyl radical which is unsubstituted or substituted by one or more radicals R^{5′}, or represents an NR^{6′}R^{7′} radical,
where the substituent(s) R^{5′} is/are each independently of one another selected from:
cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl,
and where R^{6′} and R^{7′} each independently of one another represent H, a (C₁-C₄)-alkyl, (C₁-C₄)-haloalkyl, (C₁-C₄)-alkoxy-(C₁-C₄)-alkyl radical or an unsubstituted phenyl radical or a phenyl radical substituted by halogen, (C₁-C₄)-alkyl, (C₁-C₄)-alkoxy, (C₁-C₄)-haloalkyl, (C₁-C₄)-alkoxy-(C₁-C₄)-alkyl, or
R^{6′} and R^{7′} together may form an unsubstituted or substituted 5- to 6-membered saturated or optionally fully or partially unsaturated ring which may be interrupted by 1 to 3 heteroatoms from the group consisting of oxygen, sulphur, nitrogen and which may be provided by one or more substitutions corresponding to one of the definitions of R^{5′}.

19. Compound according to claim 15 where
D′ represents a (C₁-C₆)-alkyl radical, phenyl radical, pyridine, pyrimidine, pyrazole, triazole, thiazole, oxazole, thiadiazole, oxadiazole, thiophene, pyrrole, furan, tetrahydrofuran, dioxane, isoxazole, benzyl, 2,3-dihydro-1,4-benzodioxin-5-yl, 2,3-dihydro-1-benzofuran-7-yl, quinoxalin-5-yl or indol-7-yl radical which is unsubstituted or substituted by one or more radicals R^{5′} or represents an NR^{6′} R^{7′} radical,
where the substituent(s) R^{5′} is/are each independently of one another selected from:
cyano, halogen, nitro, acetyl, hydroxy, carboxy, amino, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino and (1-pyrazolyl)-(C₁-C₃)-alkyl and
R^{6′} and R^{7′} each independently of one another represent H, a (C₁-C₆)-alkyl, (C₁-C₆)haloalkyl, (C₁-C₆)-alkoxy-(C₁-C₆)-alkyl radical or unsubstituted phenyl or halogen-, (C₁-C₆)-alkyl-, (C₁-C₆)-haloalkyl- or (C₁-C₆)-alkoxy-(C₁-C₆)-alkyl-substituted phenyl radical
or form a ring from the group pyrrolidine, morpholine, piperidine.

20. Compound according to claim 15 where
M′ represents a radical selected from the radicals of formulae (IIa) to (IIf) where
in case (IIa) R^{2′} represents H, methyl or ethyl or optionally halogen-substituted phenyl and R^{3′} represents H, methyl, ethyl, isopropyl or halogen,
in case (IIb) R^{2′} represents H, methyl or ethyl,
in case (IIc) R^{2′} represents H or methyl and R^{3′} represents H, methyl or ethyl,
in case (IId) R^{1′} represents H or methyl and R^{3′} represents H or halogen,
in case (IIe) R^{2′} represents H, methyl, methoxy, halogen-substituted phenyl or halogen-substituted pyridyl and R^{3′} represents H,
in case (IIf) R^{3′} represents H, methyl or ethyl and
Q′ represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituents R^{4′}
is/are each independently of one another selected from:
cyano, halogen, nitro, acetyl, hydroxy, carboxy, amino, SCN, tri-(C₁-C₆)-alkylsilyl, (C₃-C₈)cycloalkyl, (C₃-C₈)-cycloalkyloxy, (C₃-C₈)-cycloalkyl-(C₃-C₈)-cycloalkyl, (C₁-C₆)-alkyl(C₃-C₈)-cycloalkyl, halo-(C₃-C₈)-cycloalkyl, (C₁-C₆)-alkyl, (C₁-C₆)-haloalkyl, (C₁-C₆)cyanoalkyl, (C₁-C₆)-hydroxyalkyl, hydroxycarbonyl-(C₁-C₆)-alkoxy, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-cyanoalkenyl, (C₂-C₆)-alkynyl, (C₂-C₆)-haloalkynyl, (C₂-C₆)cyanoalkynyl, (C₁-C₆)-alkoxy, (C₁-C₆)-haloalkoxy, (C₁-C₆)-cyanoalkoxy, (C₁-C₆)alkoxycarbonyl-(C₁-C₆)-alkoxy, (C₁-C₆)-alkoxy-(C₁-C₆)-alkoxy, (C₁-C₆)alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-alkylthiocarbonyl, (C₁-C₆)-haloalkylcarbonyl, (C₁-C₆)alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)-alkylaminothiocarbonyl, di-(C₁-C₆)-alkylaminothiocarbonyl, (C₃-C₈)-cycloalkylamino, (C₁-C₆)-alkylcarbonylamino; and/or
aryl, aryloxy or hetaryl, optionally mono- or polysubstituted by identical or different substituents, where (in the case of hetaryl) optionally at least one carbonyl group may be present and where possible substituents in each case are as follows: cyano, carboxyl, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)haloalkyl-(C₁-C₆)-alkoxyimino, (C₁-C₆)-alkylthio, (C₁-C₆)-haloalkylthio, (C₁-C₆)-alkoxy(C₁-C₆)-alkylthio, (C₁-C₆)-alkylthio-(C₁-C₆)-alkyl, (C₁-C₆)-alkylsulphinyl, (C₁-C₆)haloalkylsulphinyl, (C₁-C₆)-alkoxy-(C₁-C₆)-alkylsulphinyl, (C₁-C₆)-alkylsulphinyl-(C₁-C₆)alkyl, (C₁-C₆)-alkylsulphonyl, (C₁-C₆)-haloalkylsulphonyl, (C₁-C₆)-alkoxy-(C₁-C₆)alkylsulphonyl, (C₁-C₆)-alkylsulphonyl-(C₁-C₆)-alkyl, (C₁-C₆)-alkylsulphonyloxy, (C₁-C₆)alkylcarbonyl, (C₁-C₆)-haloalkylcarbonyl, (C₁-C₆)-alkylcarbonyloxy, (C₁-C₆)alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)alkenylaminocarbonyl, (C₃-C₈)-cycloalkylaminocarbonyl, (C₁-C₆)-alkyl sulphonylamino, (C₁-C₆)-alkylamino, di-(C₁-C₆)-alkylamino, aminosulphonyl, (C₁-C₆)-alkylaminosulphonyl, di-(C₁-C₆)-alkylaminosulphonyl, (C₁-C₆)-alkylsulphoximino, aminothiocarbonyl, (C₁-C₆)alkylaminothiocarbonyl, di-(C₁-C₆)-alkylaminothiocarbonyl, (C₃-C₈)-cycloalkylamino and (C₁-C₆)-alkylcarbonylamino and
D′ represents a radical, unsubstituted or substituted by one or more radicals R^{5′}, from the group consisting of (C₁-C₆)-alkyl, phenyl and heteroaryl which may contain one to three heteroatoms from the group consisting of oxygen, sulphur and nitrogen, or, in the case that Q′ carries at least one substituent in the 2-position, represents an NR^{6′}R^{7′} radical.

21. Compound according to claim 15 where
Q′ represents a phenyl, naphth-1-yl, pyridyl, pyrimidinyl, thiophen-2-yl, benzothiophen-2-yl, benzothiophen-3-yl, isoquinolin-1-yl, benzodioxol-4-yl or pyrazol-5-yl radical which is unsubstituted or substituted by one or more radicals R^{4′}, but in cases (IIa), (IId) in the case R^{1′}=H and R^{3′}=methyl does not represent 3-methoxyphenyl, in case (IId) does not represent 3-pyridyl, in case (IIe) does not represent 2-pyrimidinyl, nor unsubstituted phenyl, nor 3,4-dichlorophenyl and
where the substituent(s) R^{4′} is/are independently of one another selected from:
cyano, halogen, nitro, acetyl, (C₃-C₆)-cycloalkyl, (C₃-C₆)-cycloalkyloxy, (C₃-C₆)cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)-hydroxyalkyl, (C₁-C₆)alkoxycarbonyl-(C₁-C₆)-alkyl, (C₁-C₆)-alkoxy-(C₁-C₆)-alkyl, (C₂-C₆)-alkenyl, (C₂-C₆)haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)-alkylsulphinyl, (C₁-C₆)haloalkylsulphinyl, (C₁-C₆)-alkylsulphonyl, (C₁-C₆)-haloalkylsulphonyl, (C₁-C₆)alkylsulphonyloxy, (C₁-C₆)-alkylcarbonyl, (C₁-C₆)-alkylamino, di-(C₁-C₆)-alkylamino, (C₁-C₆)-alkylcarbonylamino, phenyl, halophenyl, phenoxy or halophenoxy;
D′ represents a (C₁-C₆)-alkyl, phenyl, pyridine, pyrimidine, pyrazole, triazole, thiazole, oxazole, thiadiazole, oxadiazole, thiophene, pyrrole, furan, tetrahydrofuran or dioxane radical which is unsubstituted or substituted by one or more radicals R⁵,
where the substituent(s) R^{5′} is/are independently of one another selected from:
cyano, halogen, nitro, acetyl, hydroxy, carboxy, amino, (C₃-C₆)-cycloalkyl, (C₃-C₆)cycloalkyloxy, (C₃-C₆)-cycloalkyl-(C₃-C₆)-cycloalkyl, (C₁-C₆)-alkyl-(C₃-C₈)-cycloalkyl, halo-(C₃-C₆)-cycloalkyl, (C₁-C₆)-alkyl, (C₁-C₆)-haloalkyl, (C₁-C₆)-cyanoalkyl, (C₁-C₆)hydroxyalkyl, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-alkynyl, (C₁-C₆)-alkoxy, (C₁-C₆)-haloalkoxy, (C₁-C₆)-alkylthio, (C₁-C₆)-haloalkylthio, (C₁-C₆)-alkylthio-(C₁-C₆)-alkyl, (C₁-C₆)alkylsulphinyl, (C₁-C₆)-haloalkylsulphinyl, (C₁-C₆)-alkylsulphonyl, (C₁-C₆)haloalkylsulphonyl, (C₁-C₆)-alkylsulphonyloxy, (C₁-C₆)-alkylcarbonyl, (C₁-C₆)alkylamino, di-(C₁-C₆)-alkylamino, (C₁-C₆)-alkylcarbonylamino or (1-pyrazolyl) (C₁-C₃)alkyl and
R^{6′} and R^{7′} each independently of one another represent H, (C₁-C₆)-alkyl, phenyl, alkoxyphenyl or halophenyl or form a ring from the group pyrrolidine, morpholine, piperidine.

22. Compound according to claim 15 where
M′ represents a radical selected from the radicals of formulae (Va-Vz): 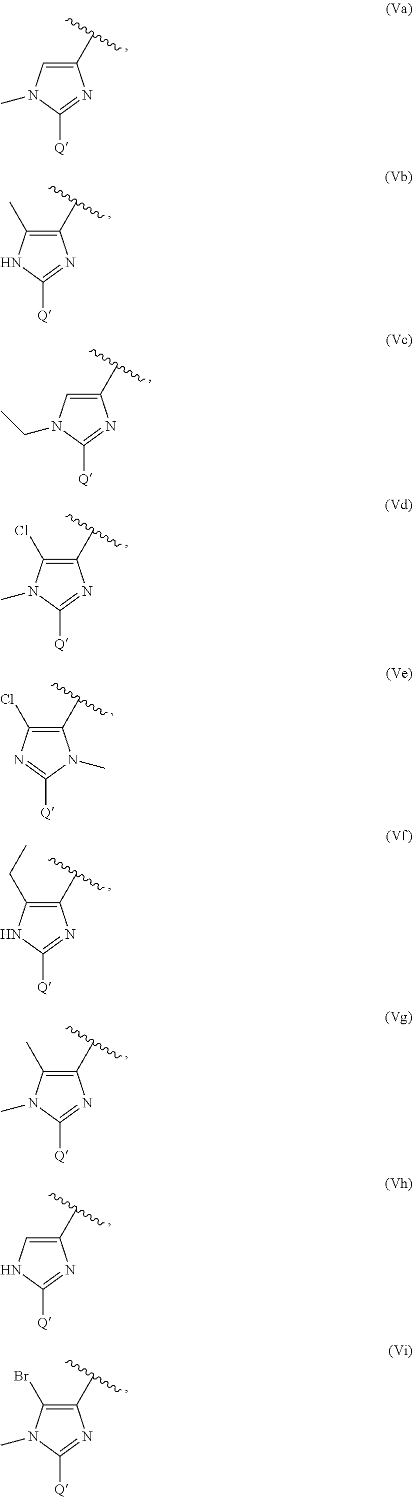 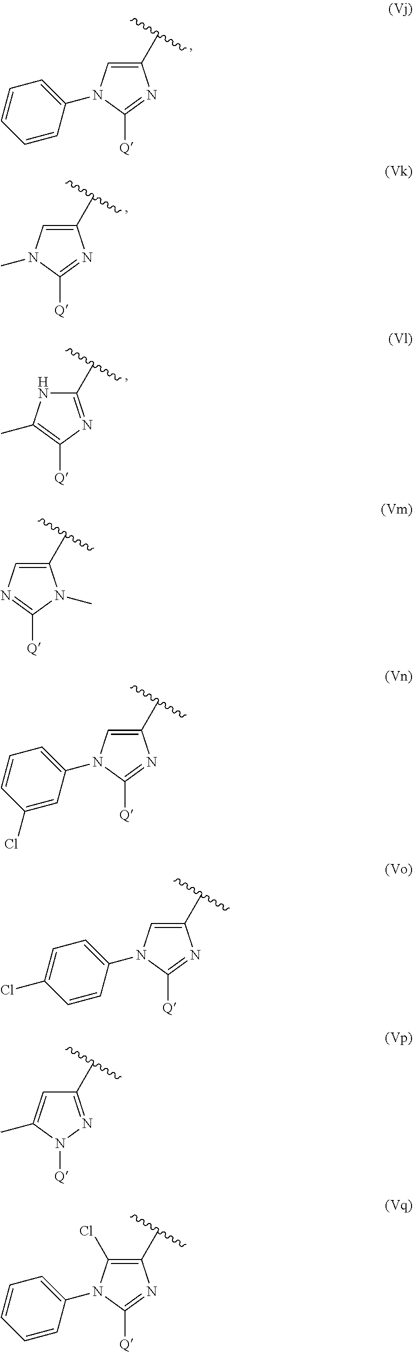 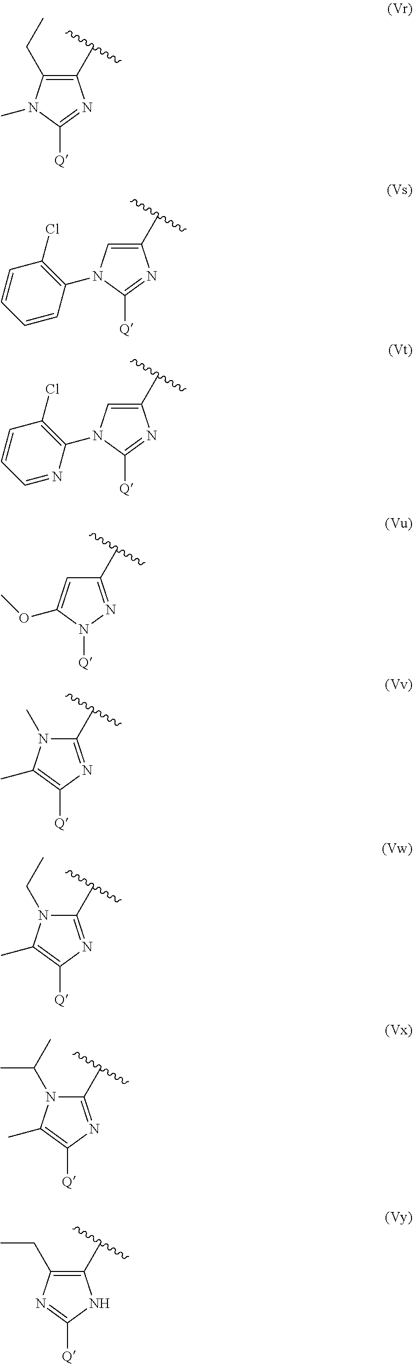 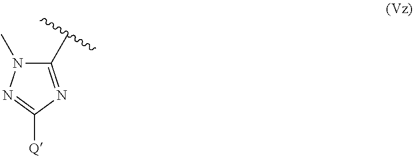
Q′ represents a radical selected from the radicals of formulae (VIa-VIz and VIa1-VIa30): 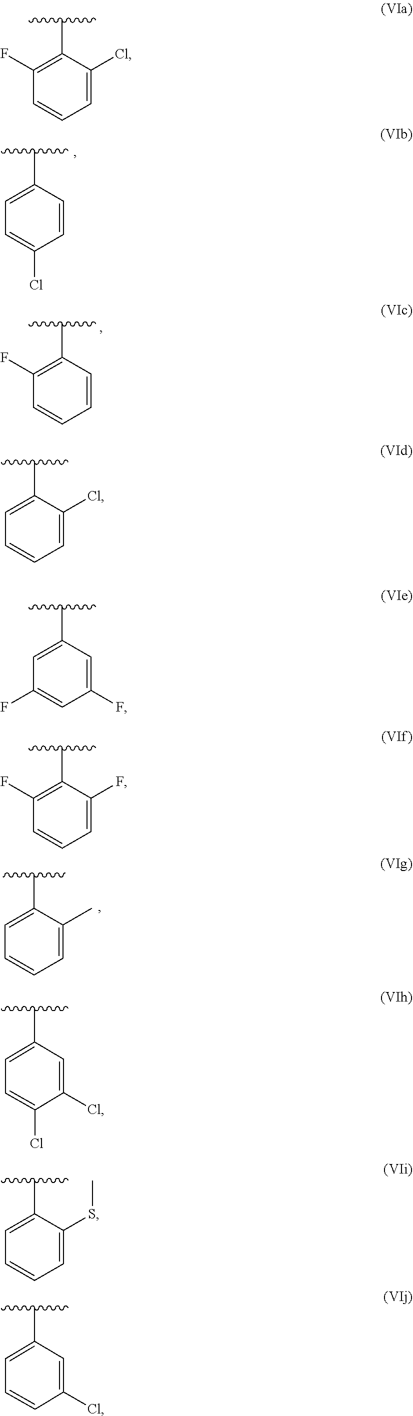 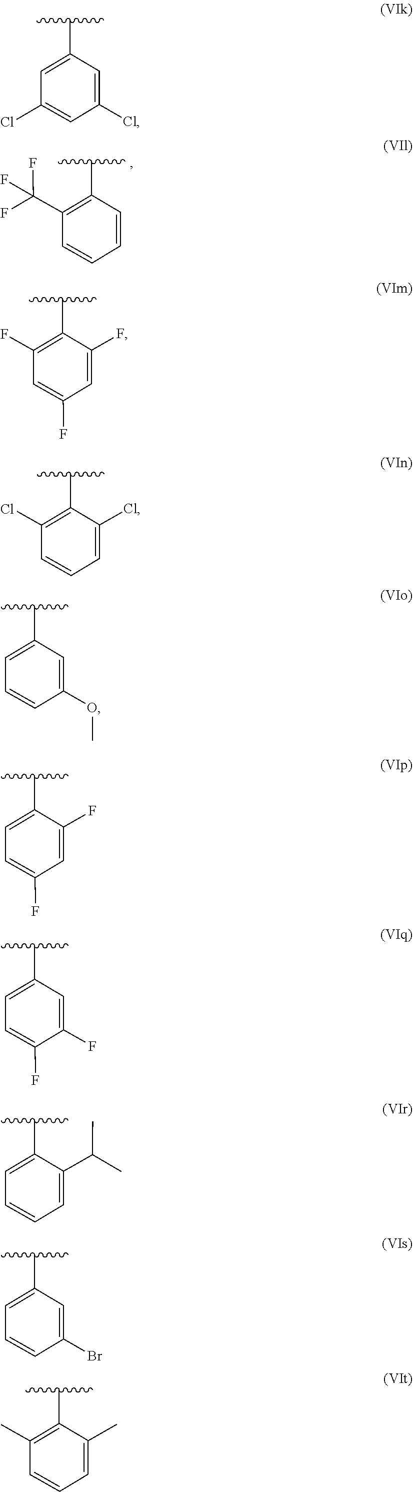 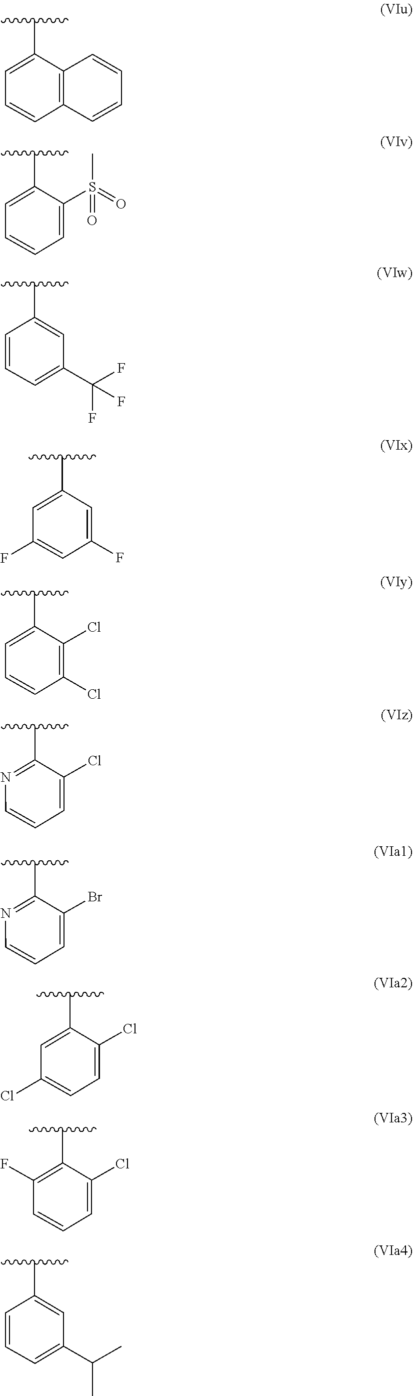 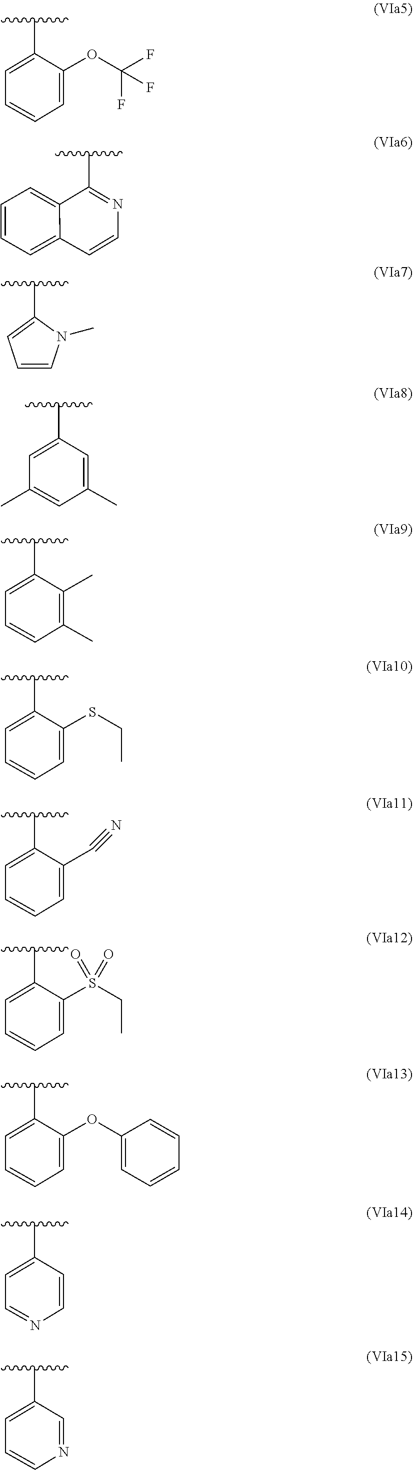 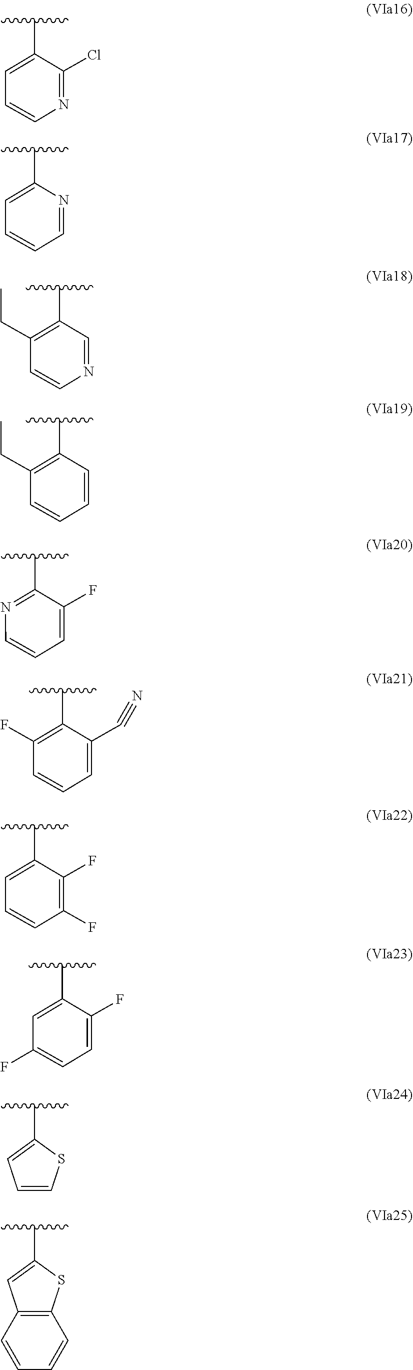 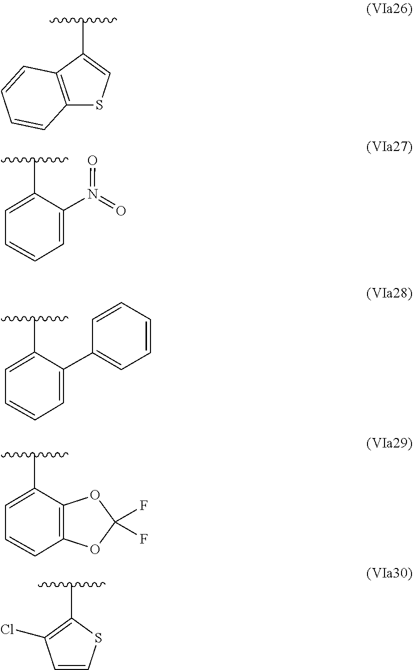
D′ represents a radical selected from the radicals of formulae (VII1-VII192)

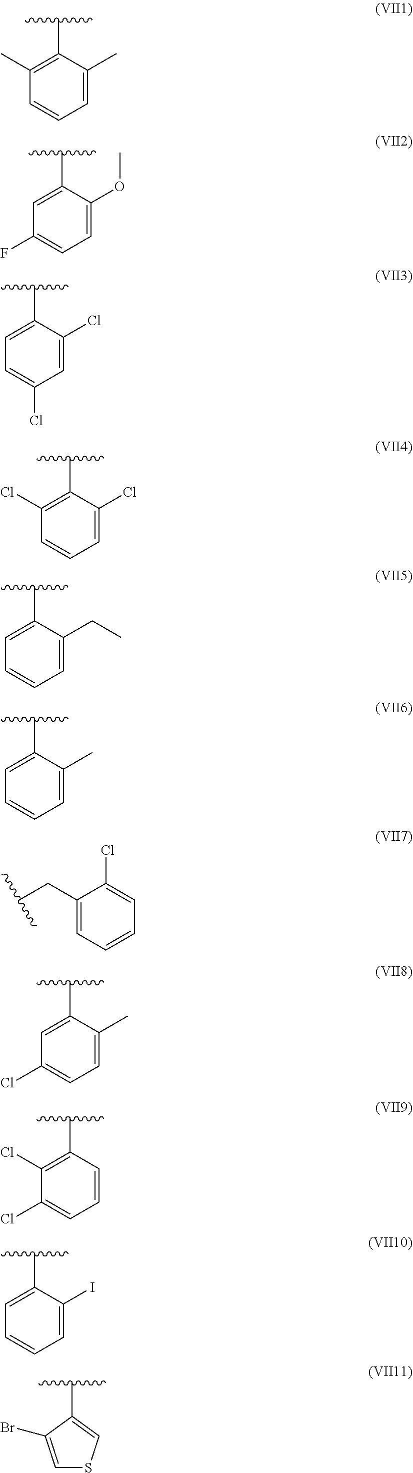

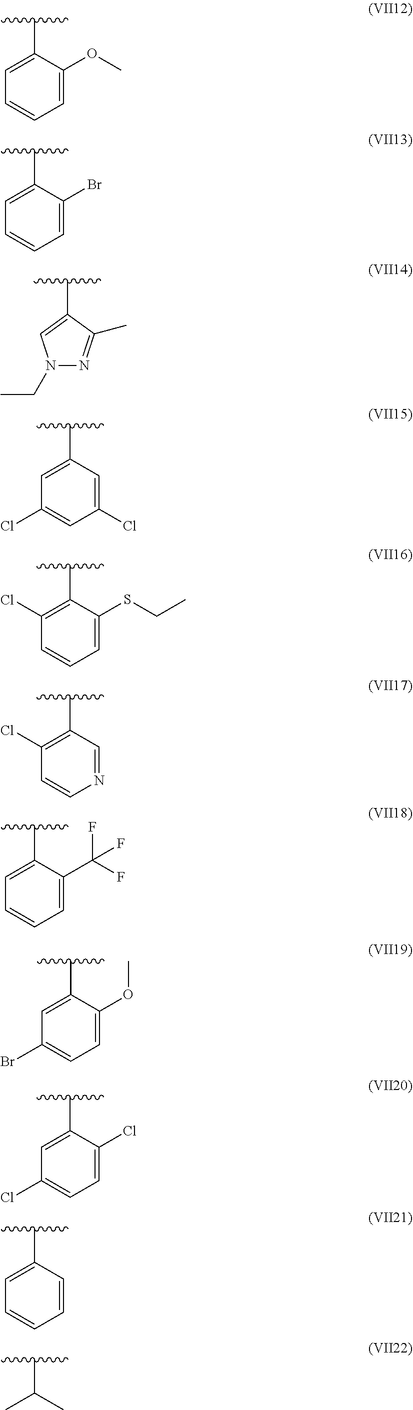

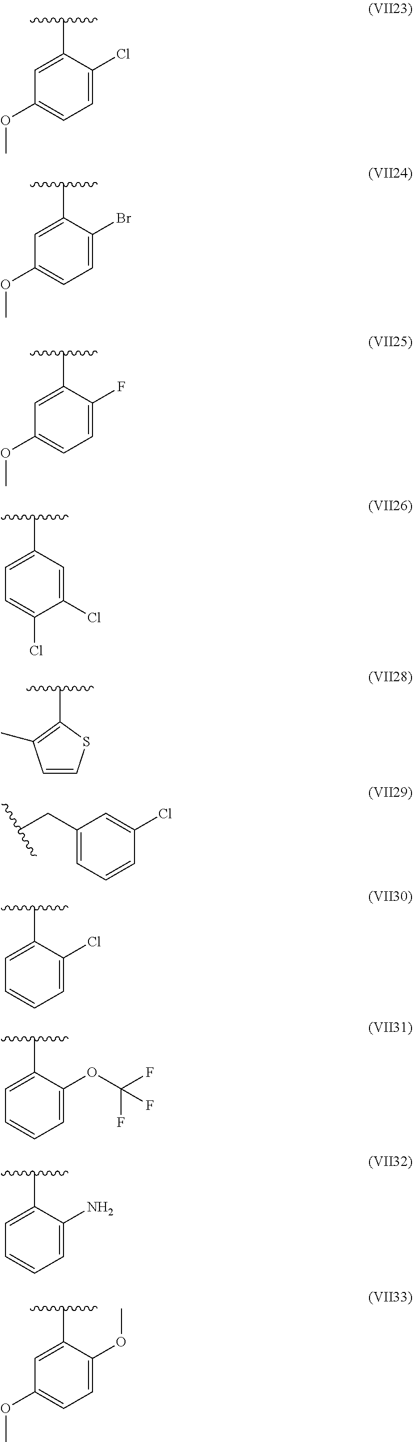

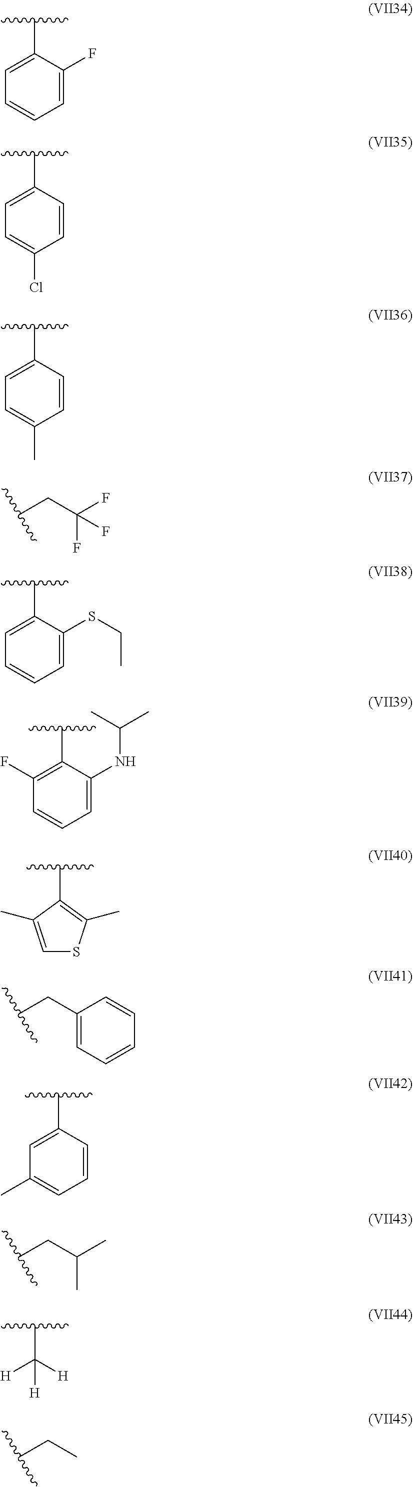

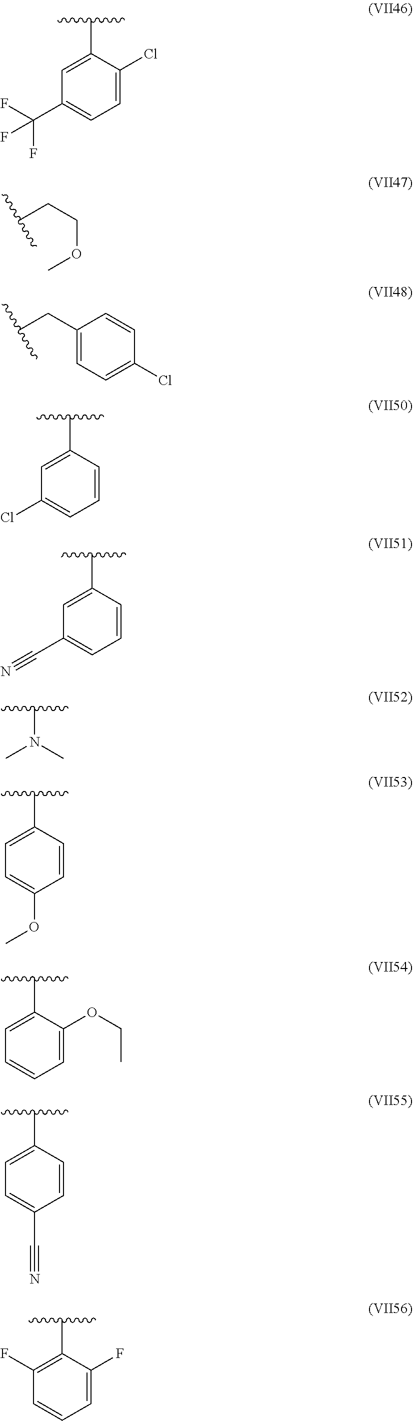

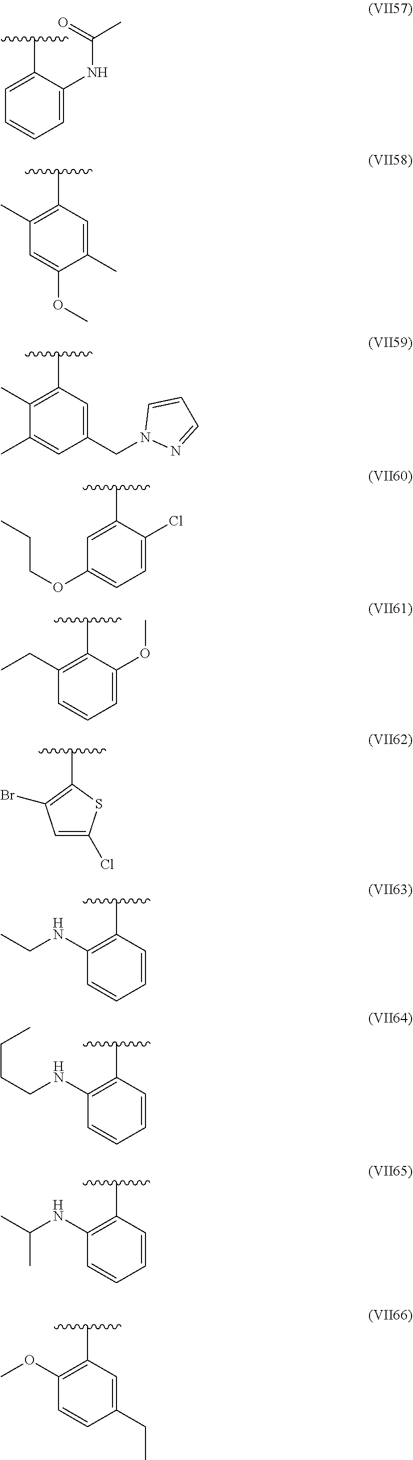

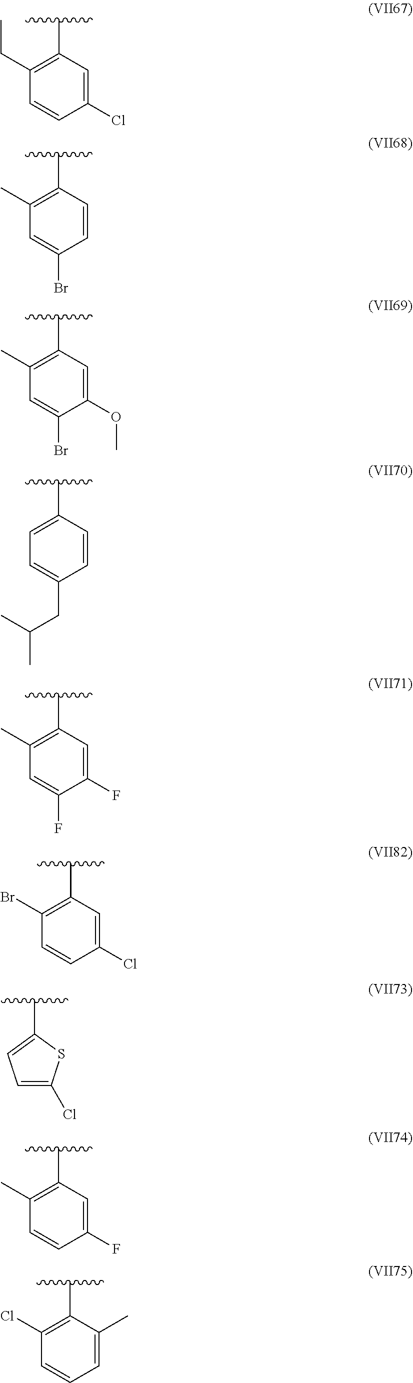

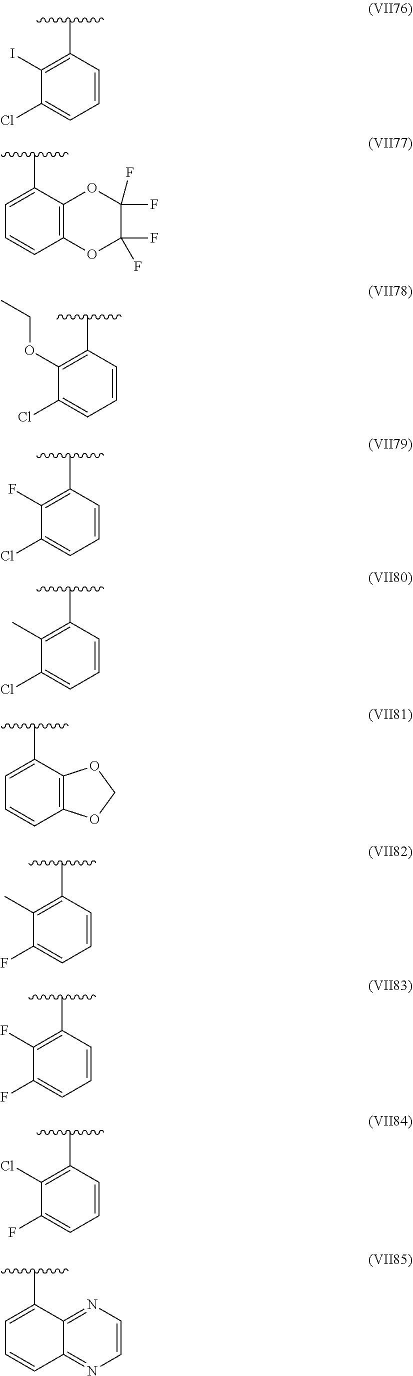

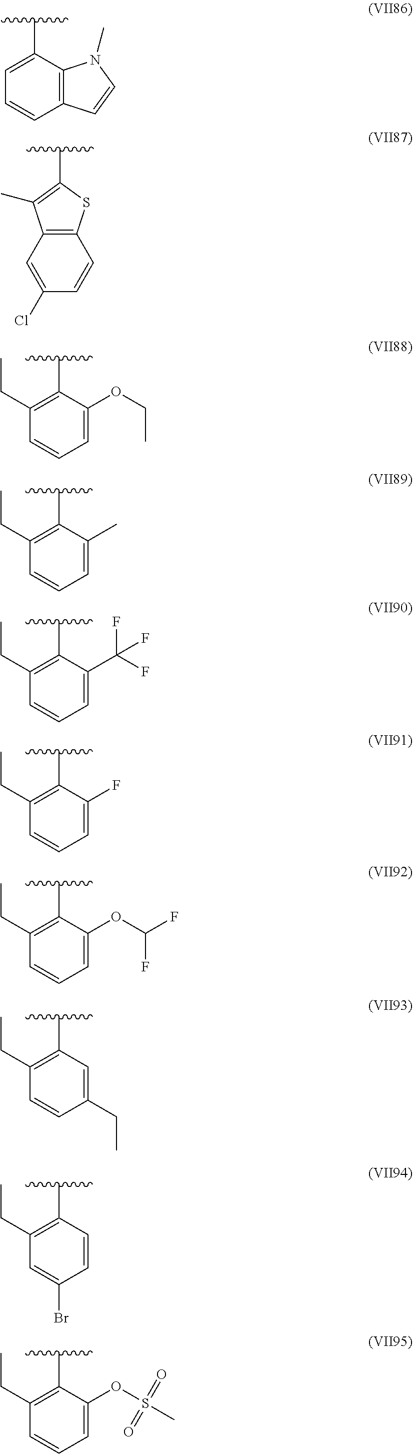

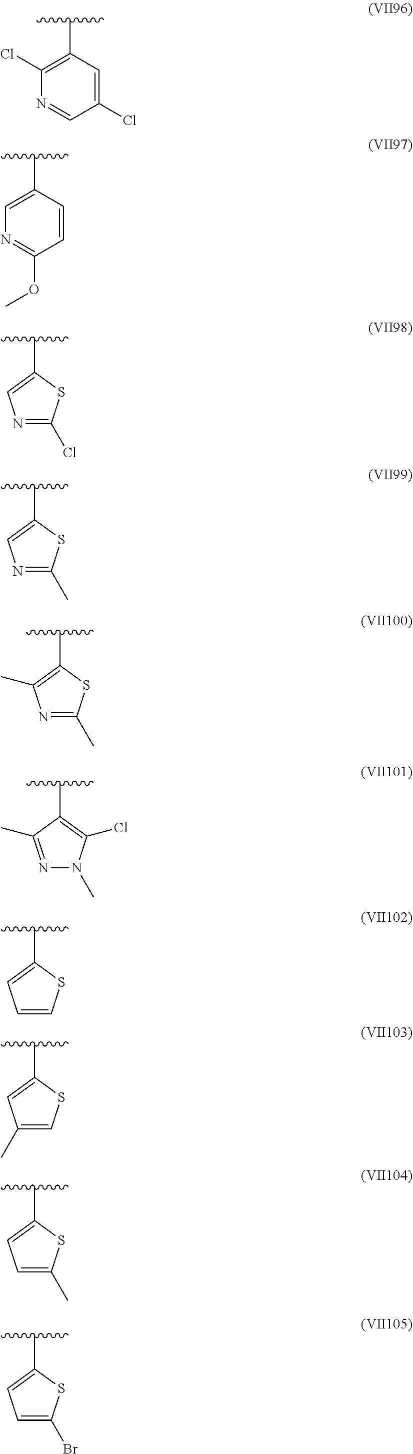

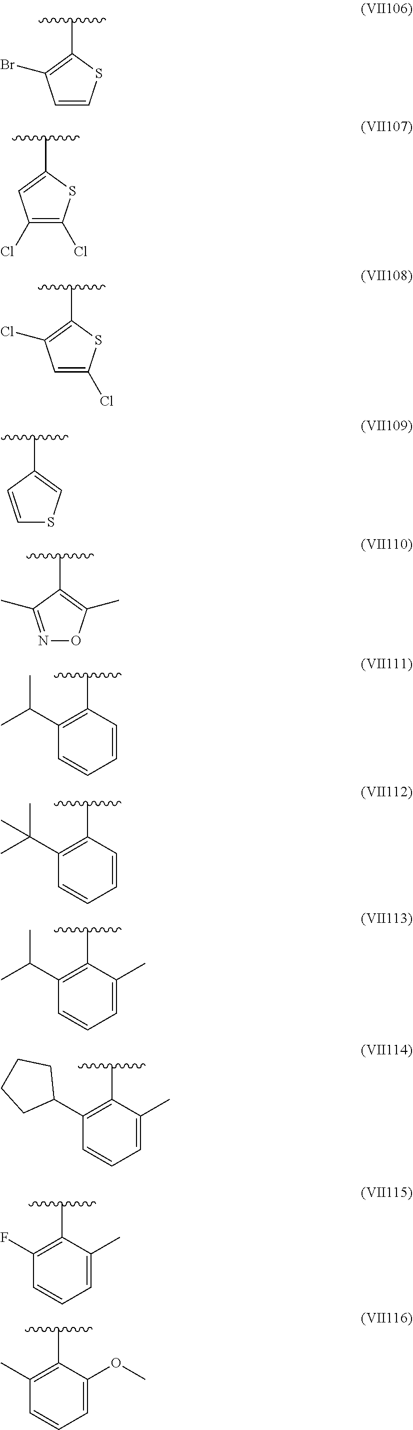

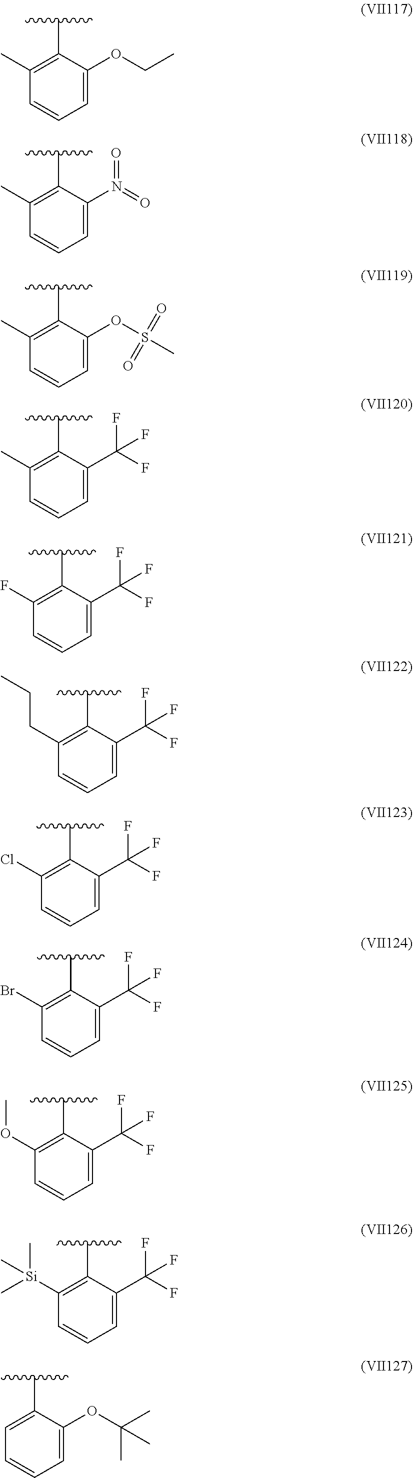

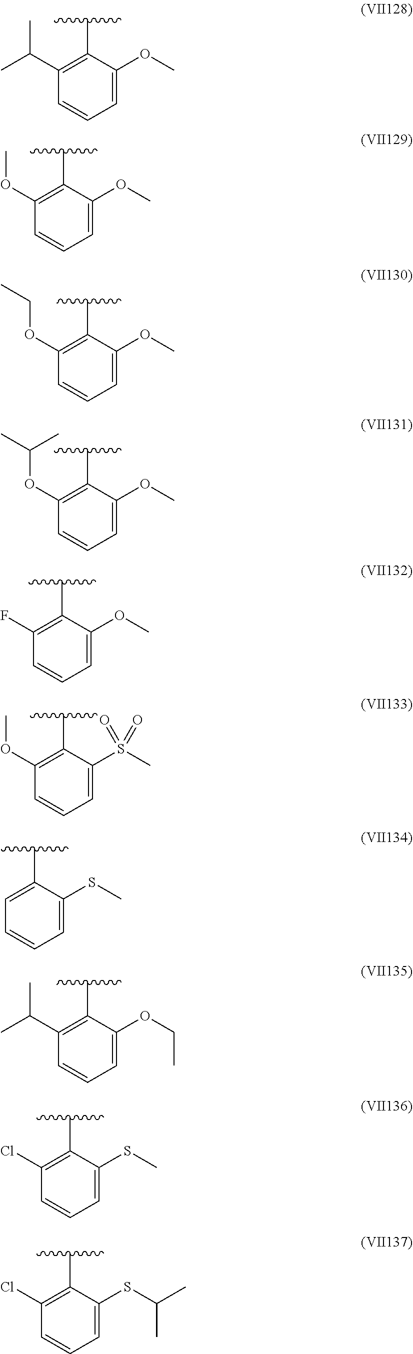

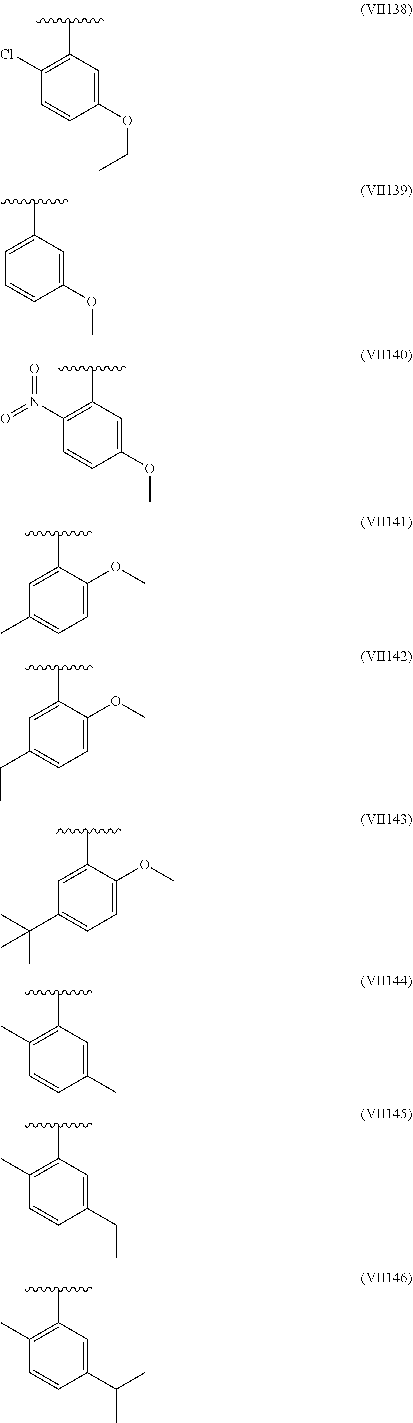

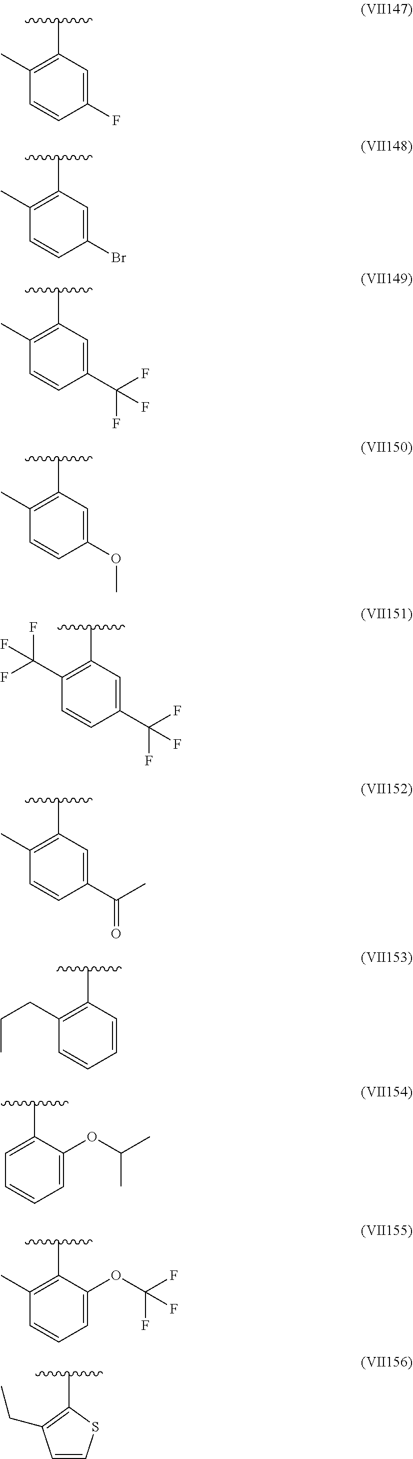

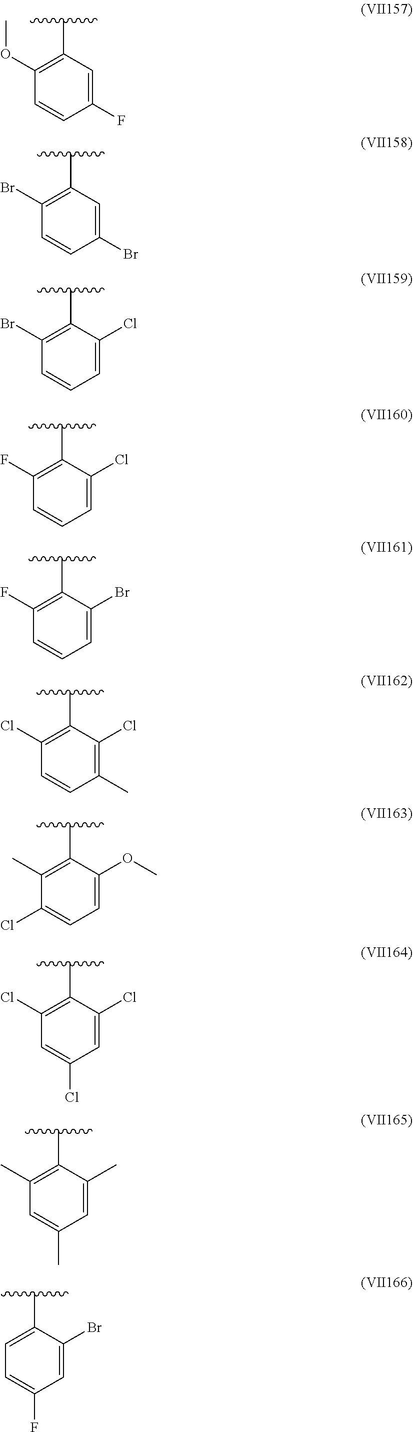

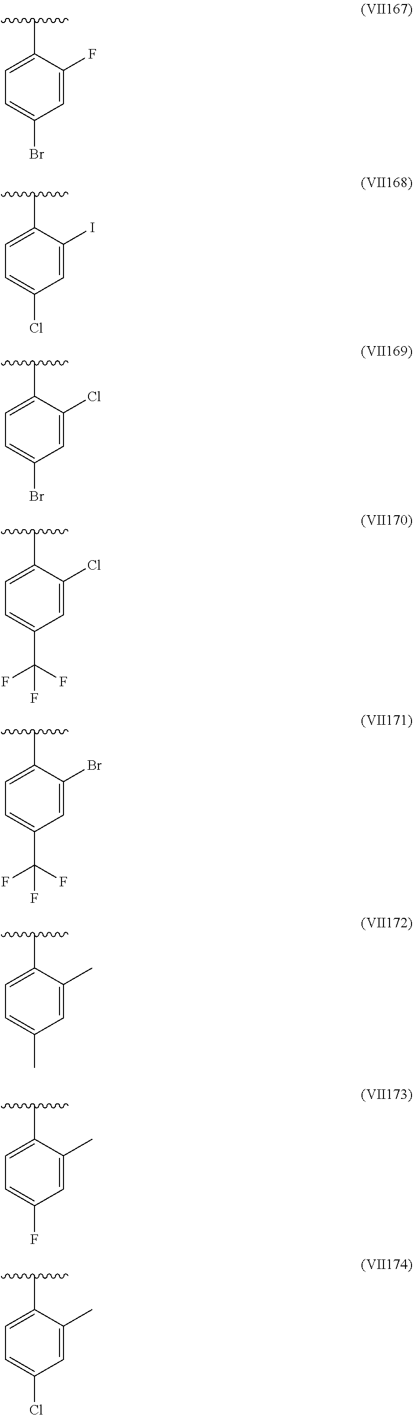

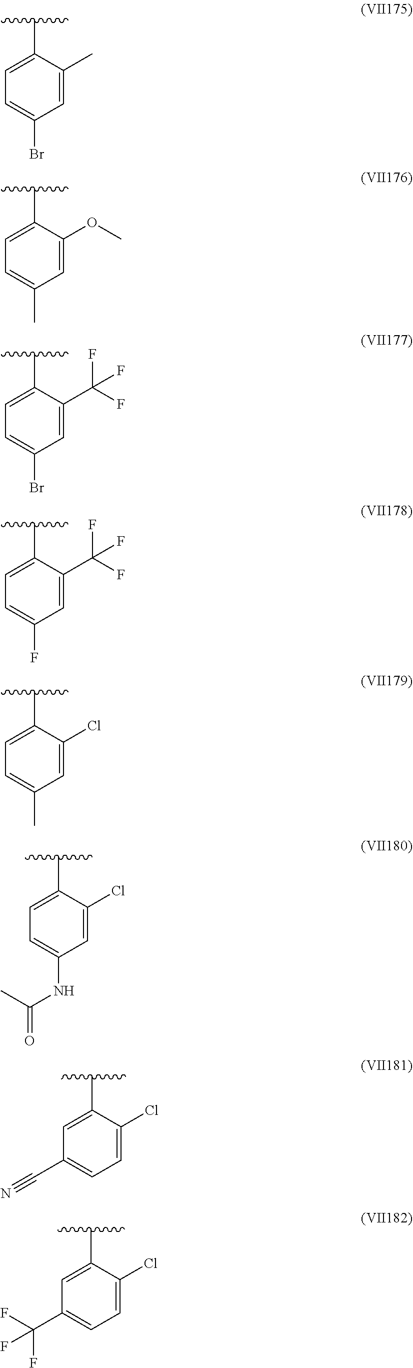

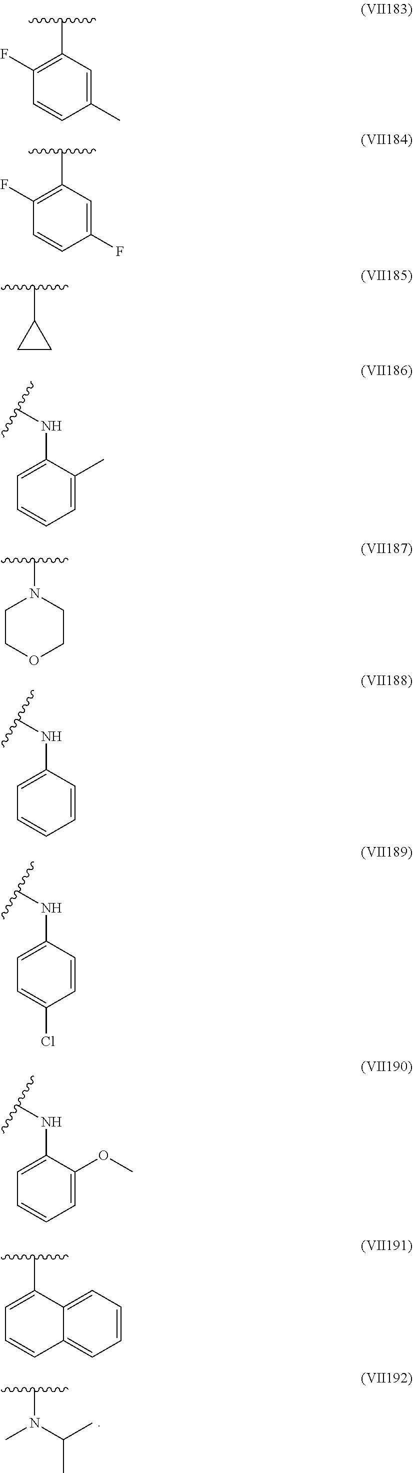

23. Intermediate of formulae XIa-XIq 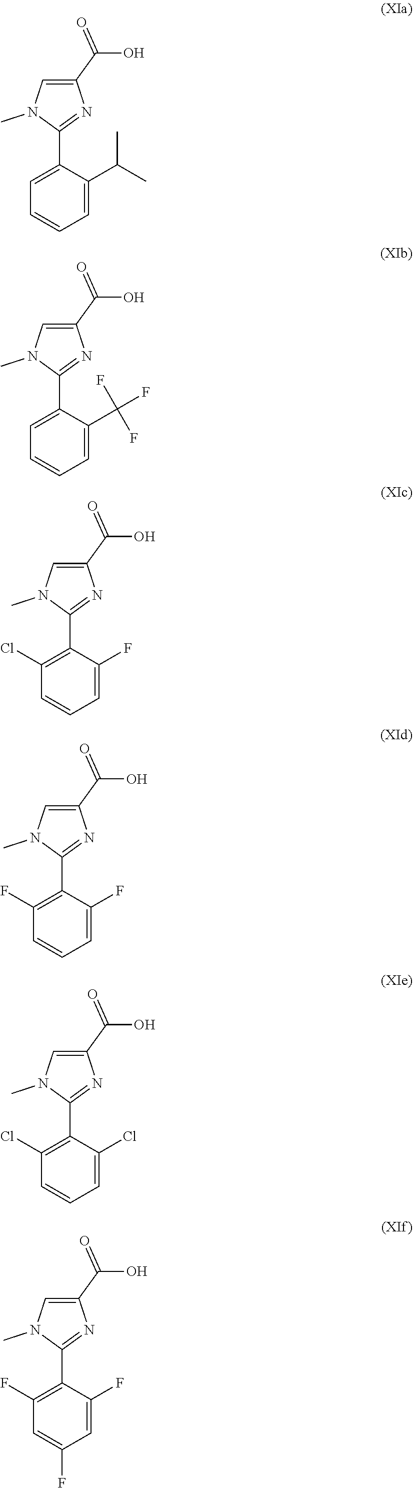 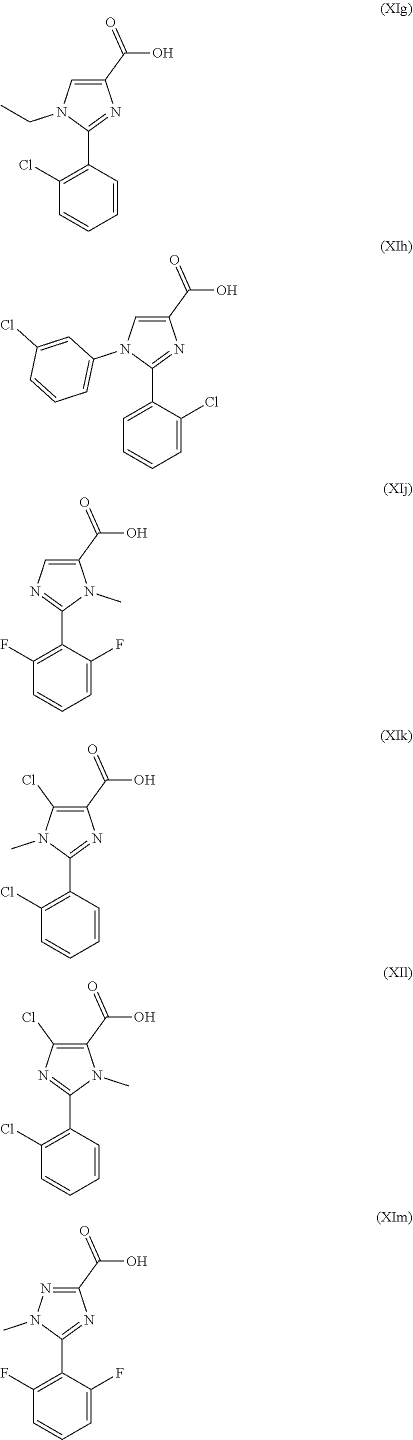 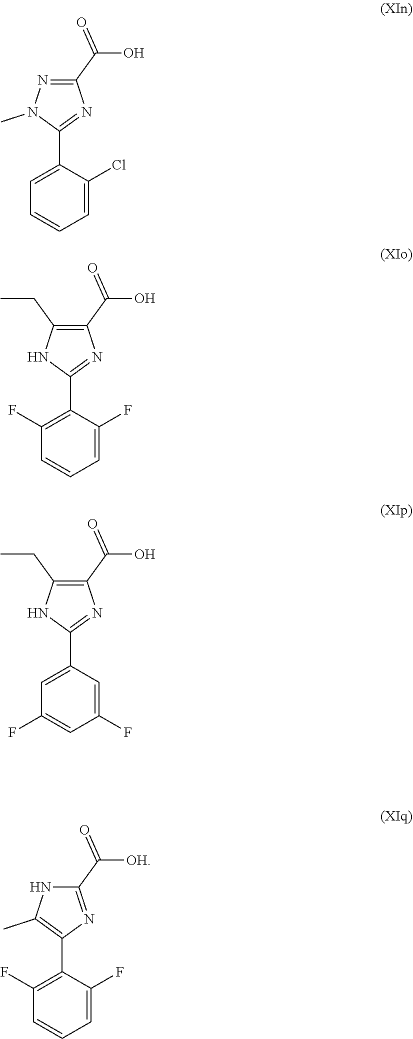 [SMILES: Cn1nc(C(=O)O)nc1-c1ccccc1Cl ; CCc1[nH]c(-c2c(F)cccc2F)nc1C(=O)O ; CCc1[nH]c(-c2cc(F)cc(F)c2)nc1C(=O)O ; Cc1[nH]c(C(=O)O)nc1-c1c(F)cccc1F]
